(19) United States
(12) Patent Application Publication
(10) Pub. No.: US 2005/0181385 A1
(43) Pub. Date: Aug. 18, 2005

(54) Synthetic lethal screen using RNA interference

(76) Inventors: Peter S. Linsley, Seattle, WA (US); Mao Mao, Redmond, WA (US); Annette S. Kim, Harleysville, PA (US); Stephen H. Friend, Philadelphia, PA (US); Steven R. Bartz, Seattle, WA (US); Michele A. Cleary, Bothell, WA (US)

Correspondence Address: JONES DAY, 222 EAST 41ST ST, NEW YORK, NY, 10017, US

(21) Appl. No.: 10/947,637

(22) Filed: Sep. 22, 2004

Related U.S. Application Data
(60) Provisional application No. 60/554,284, filed on Mar. 17, 2004.
(60) Provisional application No. 60/548,568, filed on Feb. 27, 2004.
(60) Provisional application No. 60/505,229, filed on Sep. 22, 2003.

(51) Int. Cl. C12Q 1/68; C07H 21/02; C12N 15/85

(52) U.S. Cl. 435/6; 435/455; 536/23.1

(57) ABSTRACT

The invention provides a method for identifying one or more genes in a cell of a cell type which interact with, e.g., modulate the effect of, an agent, e.g., a drug. For example, an identified gene may confer resistance or sensitivity to a drug, i.e., reduces or enhances the effect of the drug. The invention also provides STK6 and TPX2 as a gene that exhibits synthetic lethal interactions with KSP encoding a kinesin-like motor protein, and methods and compositions for treatment of diseases, e.g., cancers, by modulating the expression of STK6 or TPX2 gene and/or the activity of STK6 or TPX2 gene product. The invention also provides genes involved in cellular response to DNA damage, and their therapeutic uses.

This application claims the benefit under 35 U.S.C. § 119(e) of U.S. Provisional Patent Application No. 60/554,284, filed on Mar. 17, 2004, U.S. Provisional Patent Application No. 60/548,568, filed on Feb. 27, 2004, and U.S. Provisional Patent Application No. 60/505,229, filed on Sep. 22, 2003, each of which is incorporated by reference herein in its entirety.

### 1. FIELD OF THE INVENTION

The present invention relates to methods and compositions for carrying out interaction screening, e.g., lethal/synthetic lethal screening, using RNA interference. The invention also relates to genes exhibiting synthetic lethal interactions with KSP, a kinesin-like motor protein, and their therapeutic uses. The invention also relates to genes involved in cellular response to DNA damage, and their therapeutic uses.

### 2. BACKGROUND OF THE INVENTION

RNA interference (RNAi) is a potent method to suppress gene expression in mammalian cells, and has generated much excitement in the scientific community (Couzin, 2002, Science 298: 2296-2297; McManus et al., 2002, Nat. Rev. Genet. 3, 737-747; Hannon, G. J., 2002, Nature 418, 244-251; Paddison et al., 2002, Cancer Cell 2, 17-23). RNA interference is conserved throughout evolution, from C. elegans to humans, and is believed to function in protecting cells from invasion by RNA viruses. When a cell is infected by a dsRNA virus, the dsRNA is recognized and targeted for cleavage by an RNaseIII-type enzyme termed Dicer. The Dicer enzyme “dices” the RNA into short duplexes of 21 nt, termed siRNAs or short-interfering RNAs, composed of 19 nt of perfectly paired ribonucleotides with two unpaired nucleotides on the 3′ end of each strand. These short duplexes associate with a multiprotein complex termed RISC, and direct this complex to mRNA transcripts with sequence similarity to the siRNA. As a result, nucleases present in the RISC complex cleave the mRNA transcript, thereby abolishing expression of the gene product. In the case of viral infection, this mechanism would result in destruction of viral transcripts, thus preventing viral synthesis. Since the siRNAs are double-stranded, either strand has the potential to associate with RISC and direct silencing of transcripts with sequence similarity.

Specific gene silencing promises the potential to harness human genome data to elucidate gene function, identify drug targets, and develop more specific therapeutics. Many of these applications assume a high degree of specificity of siRNAs for their intended targets. Cross-hybridization with transcripts containing partial identity to the siRNA sequence may elicit phenotypes reflecting silencing of unintended transcripts in addition to the target gene. This could confound the identification of the gene implicated in the phenotype. Numerous reports in the literature purport the exquisite specificity of siRNAs, suggesting a requirement for near-perfect identity with the siRNA sequence (Elbashir et al., 2001. EMBO J. 20:6877-6888; Tuschl et al., 1999, Genes Dev. 13:3191-3197; Hutvagner et al., Sciencexpress 297:2056-2060). One recent report suggests that perfect sequence complementarity is required for siRNA-targeted transcript cleavage, while partial complementarity will lead to tranlational repression without transcript degradation, in the manner of microRNAs (Hutvagner et al., Sciencexpress 297:2056-2060).

The biological function of small regulatory RNAs, including siRNAs and mRNAs is not well understood. One prevailing question regards the mechanism by which the distinct silencing pathways of these two classes of regulatory RNA are determined. mRNAs are regulatory RNAs expressed from the genome, and are processed from precursor stem-loop structures to produce single-stranded nucleic acids that bind to sequences in the 3′ UTR of the target mRNA (Lee et al., 1993, Cell 75:843-854; Reinhart et al., 2000, Nature 403:901-906; Lee et al., 2001, Science 294:862-864; Lau et al., 2001, Science 294:858-862; Hutvagner et al., 2001, Science 293:834-838). mRNAs bind to transcript sequences with only partial complementarity (Zeng et al., 2002, Molec. Cell 9:1327-1333) and repress translation without affecting steady-state RNA levels (Lee et al., 1993, Cell 75:843-854; Wightman et al., 1993, Cell 75:855-862). Both mRNAs and siRNAs are processed by Dicer and associate with components of the RNA-induced silencing complex (Hutvagner et al., 2001, Science 293:834-838; Grishok et al., 2001, Cell 106: 23-34; Ketting et al., 2001, Genes Dev. 15:2654-2659; Williams et al., 2002, Proc. Natl. Acad. Sci. USA 99:6889-6894; Hammond et al., 2001, Science 293:1146-1150; Mourlatos et al., 2002, Genes Dev. 16:720-728). A recent report (Hutvagner et al., 2002, Sciencexpress 297:2056-2060) hypothesizes that gene regulation through the mRNA pathway versus the siRNA pathway is determined solely by the degree of complementarity to the target transcript. It is speculated that siRNAs with only partial identity to the mRNA target will function in translational repression, similar to an mRNA, rather than triggering RNA degradation.

It has also been shown that siRNA and shRNA can be used to silence genes in vivo. The ability to utilize siRNA and shRNA for gene silencing in vivo has the potential to enable selection and development of siRNAs for therapeutic use. A recent report highlights the potential therapeutic application of siRNAs. Fas-mediated apoptosis is implicated in a broad spectrum of liver diseases, where lives could be saved by inhibiting apoptotic death of hepatocytes. Song (Song et al. 2003, Nat. Medicine 9, 347-351) injected mice intravenously with siRNA targeted to the Fas receptor. The Fas gene was silenced in mouse hepatocytes at the mRNA and protein levels, prevented apoptosis, and protected the mice from hepatitis-induced liver damage. Thus, silencing Fas expression holds therapeutic promise to prevent liver injury by protecting hepatocytes from cytotoxicity. As another example, injected mice intraperitoneally with siRNA targeting TNF-a. Lipopolysaccharide-induced TNF-a gene expression was inhibited, and these mice were protected from sepsis. Collectively, these results suggest that siRNAs can function in vivo, and may hold potential as therapeutic drugs (Sorensen et al., 2003, J. Mol. Biol. 327, 761-766).

Martinez et al. reported that RNA interference can be used to selectively target oncogenic mutations (Martinez et al., 2002, Proc. Natl. Acad. Sci. USA 99:14849-14854). In this report, an siRNA that targets the region of the R248W mutant of p53 containing the point mutation was shown to silence the expression of the mutant p53 but not the wild-type p53.

Wilda et al. reported that an siRNA targeting the M-BCR/ABL fusion mRNA can be used to deplete the M-BCR/ABL mRNA and the M-BRC/ABL oncoprotein in leukemic cells (Wilda et al., 2002, Oncogene 21:5716-5724). However, the report also showed that applying the siRNA in combination with Imatinib, a small-molecule ABL kinase tyrosine inhibitor, to leukemic cells did not further increase in the induction of apoptosis.

U.S. Pat. No. 6,506,559 discloses a RNA interference process for inhibiting expression of a target gene in a cell. The process comprises introducing partially or fully doubled-stranded RNA having a sequence in the duplex region that is identical to a sequence in the target gene into the cell or into the extracellular environment. RNA sequences with insertions, deletions, and single point mutations relative to the target sequence are also found as effective for expression inhibition.

U.S. Patent Application Publication No. U.S. 2002/0086356 discloses RNA interference in a Drosophila in vitro system using RNA segments 21-23 nucleotides (nt) in length. The patent application publication teaches that when these 21-23 nt fragments are purified and added back to Drosophila extracts, they mediate sequence-specific RNA interference in the absence of long dsRNA. The patent application publication also teaches that chemically synthesized oligonucleotides of the same or similar nature can also be used to target specific mRNAs for degradation in mammalian cells.

PCT publication WO 02/44321 discloses that double-stranded RNA (dsRNA) 19-23 nt in length induces sequence-specific post-transcriptional gene silencing in a Drosophila in vitro system. The PCT publication teaches that short interfering RNAs (siRNAs) generated by an RNase III-like processing reaction from long dsRNA or chemically synthesized siRNA duplexes with overhanging 3′ ends mediate efficient target RNA cleavage in the lysate, and the cleavage site is located near the center of the region spanned by the guiding siRNA. The PCT publication also provides evidence that the direction of dsRNA processing determines whether sense or antisense target RNA can be cleaved by the produced siRNP complex.

U.S. Patent Application Publication No. U.S. 2002/016216 discloses a method for attenuating expression of a target gene in cultured cells by introducing double stranded RNA (dsRNA) that comprises a nucleotide sequence that hybridizes under stringent conditions to a nucleotide sequence of the target gene into the cells in an amount sufficient to attenuate expression of the target gene.

PCT publication WO 03/006477 discloses engineered RNA precursors that when expressed in a cell are processed by the cell to produce targeted small interfering RNAs (siRNAs) that selectively silence targeted genes (by cleaving specific mRNAs) using the cell's own RNA interference (RNAi) pathway. The PCT publication teaches that by introducing nucleic acid molecules that encode these engineered RNA precursors into cells in vivo with appropriate regulatory sequences, expression of the engineered RNA precursors can be selectively controlled both temporally and spatially, i.e., at particular times and/or in particular tissues, organs, or cells.

Discussion or citation of a reference herein shall not be construed as an admission that such reference is prior art to the present invention.

### 3. SUMMARY OF THE INVENTION

The invention provides methods and compositions for identifying interactions, e.g., lethal/synthetic lethal interactions, between a gene or its product and an agent, e.g., a drug, and/or another gene or its product, using RNA interference. The invention also provides methods and compositions for treating cancer utilizing the synthetic lethal interaction between STK6 kinase or TPX2 and kinesin-like motor protein KSP inhibitors. The invention also provides genes involved in cellular response to DNA damage, and their therapeutic uses.

In one aspect, the invention provides a method for identifying a gene whose product modulates the effect of an agent on a cell of a cell type. The method comprises (a) contacting a plurality of groups of one or more cells of said cell type with said agent, wherein each said group of one or more cells comprises one or more different small interfering RNAs (siRNAs) from among a plurality of different siRNAs, said one or more different siRNAs targeting a same gene, and said plurality of different siRNAs comprising siRNAs targeting respectively different genes in cells of said cell type; (b) comparing the effect of said agent on each said group of one or more cells to the effect of said agent on a cell of said cell type which does not comprise an siRNA targeting any one of said different genes; and (c) identifying a gene as said gene whose product modulates the effect of said agent on a cell of said cell type if the effect of said agent on said group of one or more cells comprising said one or more different siRNAs targeting said gene is different as compared to the effect of said agent on a cell of said cell type which does not comprise an siRNA targeting any one of said different genes. In one embodiment, each said group of cells comprising one or more of said plurality of siRNAs is obtained by transfection with said one or more siRNAs prior to said step of contacting. In one embodiment, the contacting step (a) is carried out separately for each said groups of one or more cells.

In a specific embodiment, the invention provides a method for identifying a gene whose product modulates the effect of an agent on a cell of a cell type, said method comprising (a) transfacting each of a plurality of groups of one or more cells of said cell type with a composition comprising one or more different small interfering RNAs (siRNAs) from among a plurality of different siRNAs, said one or more different siRNAs targeting a same gene, and said plurality of different siRNAs comprising siRNAs targeting respectively different genes in cells of said cell type; (b) contacting each of said plurality of groups of one or more cells with said agent; (c) comparing the effect of said agent on each said group of one or more cells to the effect of said agent on a cell of said cell type which is not transfected with an siRNA targeting any one of said different genes; and (d) identifying a gene as said gene whose product modulates the effect of said agent on a cell of said cell type if the effect of said agent on said group of one or more cells comprising said one or more different siRNAs targeting said gene is different as compared to the effect of said agent on a cell of said cell type which does not comprise an siRNA targeting any one of said different genes.

The effect of said agent on each said group of one or more cells comprising said one or more different siRNAs can be enhanced as compared to the effect of said agent on a cell of said cell type which does not comprise an siRNA targeting any one of said different genes. Alternatively, the effect of said agent on said group of one or more cells comprising said one or more different siRNAs can be reduced as compared to the effect of said agent on a cell of said cell type which does not comprise an siRNA targeting any one of said different genes.

Preferably, the agent acts on a gene other than any one of said different genes targeted by said plurality of siRNAs, or a protein encoded thereof. Preferably, the plurality of siRNAs comprises at least k different siRNAs targeting at least one of said different genes, wherein said k is selected from the group consisting of 2, 3, 4, 5, 6 and 10. More preferably, the plurality of siRNAs comprises at least k different siRNAs targeting each of at least 2 different genes of said different genes, wherein said k is selected from the group consisting of 2, 3, 4, 5, 6 and 10. Still more preferably, the plurality of siRNAs comprises at least k different siRNAs targeting each of said different genes, wherein said k is selected from the group consisting of 2, 3, 4, 5, 6 and 10.

Preferably, the one or more different siRNAs for at least one, at least two, or each of of the plurality of different genes comprises 2, 3, 4, 5, 6, or 10 different siRNAs targeting a same target gene. In a preferred embodiment, the total siRNA concentration of the one or more siRNAs is about the same as the concentration of a single siRNA when used individually, e.g., 100 nM. Preferably, the total concentration of the one or more siRNAs is an optimal concentration for silencing the intended target gene. An optimal concentration is a concentration further increase of which does not increase the level of silencing substantially. In one embodiment, the optimal concentration is a concentration further increase of which does not increase the level of silencing by more than 5%, 10% or 20%. In a preferred embodiment, the one or more siRNAs comprise each siRNA in equal proportion. In another preferred embodiment, the one or more siRNAs comprise each siRNA in proportions different from each other by less than 5%, 10%, 20% or 50%. In a preferred embodiment, at least one of the one or more siRNAs constitutes more than 90%, 80%, 70%, 50%, or 20% of the total siRNA concentration of the one or more siRNAs. In another preferred embodiment, none of the siRNAs in the one or more siRNAs constitutes more than 90%, 80%, 70%, 50%, or 20% of the total siRNA concentration of the one or more siRNAs. In a preferred embodiment, the composition of the one or more siRNAs, including the number of different siRNAs and the concentration of each siRNA, is chosen such that the one or more siRNAs causes less than 30%, 20%, 10% or 5%, 1%, 0.1% or 0.01% of silencing of any off-target genes. In other embodiments, each siRNA in the one or more siRNAs has a concentration that is lower than the optimal concentration when used individually. In a preferred embodiment, at least one siRNA in the one or more siRNAs has an concentration that is lower than the concentration of the siRNA that is effective to achieve at least 30%, 50%, 75%, 80%, 85%, 90% or 95% silencing when used in the absence of other siRNAs or in the absence of other siRNAs designed to silence the gene. In another preferred embodiment, each different siRNA in the one or more siRNAs has a concentration that causes less than 30%, 20%, 10% or 5% of silencing of the gene when used in the absence of other siRNAs or in the absence of other siRNAs designed to silence the gene. In a preferred embodiment, each siRNA has a concentration that causes less than 30%, 20%, 10% or 5% of silencing of the target gene when used alone, while the plurality of siRNAs causes at least 80% or 90% of silencing of the target gene.

In one embodiment, said cell type is a cancer cell type. In another embodiment, said effect is growth inhibitory effect. In a specific embodiment, said agent is a KSP inhibitor. In preferred embodiments, said different genes comprises at least 5, at least 10, at least 100, or at least 1,000 different genes. In one embodiment, said different genes are different endogenous genes.

In another aspect, the invention provides a method for identifying a gene which interacts with a primary target gene in a cell of a cell type. The method comprises (a) contacting a plurality of groups of one or more cells of said cell type with an agent, wherein said agent modulates the expression of said primary target gene and/or the activity of a protein encoded by said primary target gene, and wherein each said group of cells comprises one or more different siRNAs among a plurality of different siRNAs, said one or more different siRNAs targeting a same gene, and said plurality of different siRNAs comprising siRNAs targeting respectively different secondary genes in said cell; (b) comparing the effect of said agent on each said group of one or more cells to the effect of said agent on a cell of said cell type which does not comprise an siRNA targeting any one of said different secondary genes; and (c) identifying a gene as said gene that interacts with said primary target gene in a cell of said cell type if the effect of said agent on said group of one or more cells comprising one or more siRNAs targeting said gene is different as compared to the effect of said agent on a cell of said cell type which does not comprise an siRNA targeting any one of said different secondary genes. In one embodiment, each said group of cells comprising one or more of said plurality of siRNAs is obtained by transfection with said one or more siRNA prior to said step of contacting.

In a specific embodiment, the invention provides method for identifying a gene which interacts with a primary target gene in a cell of a cell type, said method comprising (a) transfacting each of a plurality of groups of one or more cells of said cell type with a composition comprising one or more different small interfering RNAs (siRNAs) from among a plurality of different siRNAs, said one or more different siRNAs targeting a same gene, and said plurality of different siRNAs comprising siRNAs targeting respectively different genes in cells of said cell type; (b) contacting said plurality of groups of one or more cells of said cell type with an agent, wherein said agent modulates the expression of said primary target gene and/or the activity of a protein encoded by said primary target gene; (c) comparing the effect of said agent on each said group of one or more cells to the effect of said agent on a cell of said cell type which does not comprise an siRNA targeting any one of said different secondary genes; and (d) identifying a gene as said gene that interacts with said primary target gene in a cell of said cell type if the effect of said agent on said group of one or more cells comprising one or more siRNAs targeting said gene is different as compared to the effect of said agent on a cell of said cell type which does not comprise an siRNA targeting any one of said different secondary genes.

In one embodiment, said agent comprises an siRNA targeting and silencing said primary target gene. In another embodiment, said agent comprises 2, 3, 4, 5, 6, or 10 different siRNAs targeting said primary target gene. In a preferred embodiment, each of said different siRNAs targeting said primary target gene. In a preferred embodiment, the total siRNA concentration of said different siRNAs is about the same as the concentration of a single siRNA when used individually, e.g., 100 nM. Preferably, the total concentration of said different siRNAs is an optimal concentration for silencing the primary target gene. An optimal concentration is a concentration further increase of which does not increase the level of silencing substantially. In one embodiment, the optimal concentration is a concentration further increase of which does not increase the level of silencing by more than 5%, 10% or 20%. In a preferred embodiment, the different siRNAs comprise each siRNA in equal proportion. In another preferred embodiment, the different siRNAs comprise each siRNA in proportions different from each other by less than 5%, 10%, 20% or 50%. In a preferred embodiment, at least one of the different siRNAs constitutes more than 90%, 80%, 70%, 50%, or 20% of the total siRNA concentration of the different siRNAs. In another preferred embodiment, none of the siRNAs constitutes more than 90%, 80%, 70%, 50%, or 20% of the total siRNA concentration of the different siRNAs. In a preferred embodiment, the composition of the different siRNAs, including the number of different siRNAs and the concentration of each siRNA, is chosen such that the different siRNAs causes less than 30%, 20%, 10% or 5%, 1%, 0.1% or 0.01% of silencing of any off-target genes. In other embodiments, each siRNA has a concentration that is lower than the optimal concentration when used individually. In a preferred embodiment, at least one siRNA has an concentration that is lower than the concentration of the siRNA that is effective to achieve at least 30%, 50%, 75%, 80%, 85%, 90% or 95% silencing when used in the absence of other siRNAs or in the absence of other siRNAs designed to silence the gene. In another preferred embodiment, each different siRNA has a concentration that causes less than 30%, 20%, 0.10% or 5% of silencing of the gene when used in the absence of other siRNAs or in the absence of other siRNAs designed to silence the gene. In a preferred embodiment, each siRNA has a concentration that causes less than 30%, 20%, 10% or 5% of silencing of the target gene when used alone, while all of the siRNAs together causes at least 80% or 90% of silencing of the target gene. In still another embodiment, said agent comprises an inhibitor of a protein encoded by said primary target gene.

The effect of said agent on said group of one or more cells can be enhanced as compared to the effect of said agent on a cell of said cell type which does not comprise an siRNA targeting any one of said different secondary genes. Alternatively, the effect of said agent on said group of one or more cells can be reduced as compared to the effect of said agent on a cell of said cell type which does not comprise an siRNA targeting any one of said different secondary genes.

Preferably, the plurality of siRNAs comprises at least k different siRNAs targeting at least one of said different secondary genes, wherein said k is selected from the group consisting of 2, 3, 4, 5, 6 and 10. More preferably, the plurality of siRNAs comprises at least k different siRNAs targeting each of at least 2 different genes of said different secondary genes, wherein said k is selected from the group consisting of 2, 3, 4, 5, 6 and 10. Still more preferably, the plurality of siRNAs comprises at least k different siRNAs targeting each of said different secondary genes, wherein said k is selected from the group consisting of 2, 3, 4, 5, 6 and 10.

Preferably, the one or more different siRNAs for at least one, at least two, or each of of the plurality of different genes comprises 2, 3, 4, 5, 6, or 10 different siRNAs targeting a same target gene. In a preferred embodiment, the total siRNA concentration of the one or more siRNAs targeting a same gene is about the same as the concentration of a single siRNA when used individually, e.g., 100 nM. Preferably, the total concentration of the one or more siRNAs is an optimal concentration for silencing the intended target gene. An optimal concentration is a concentration further increase of which does not increase the level of silencing substantially. In one embodiment, the optimal concentration is a concentration further increase of which does not increase the level of silencing by more than 5%, 10% or 20%. In a preferred embodiment, the one or more siRNAs comprise each siRNA in equal proportion. In another preferred embodiment, the one or more siRNAs comprise each siRNA in proportions different from each other by less than 5%, 10%, 20% or 50%. In a preferred embodiment, at least one of the one or more siRNAs constitutes more than 90%, 80%, 70%, 50%, or 20% of the total siRNA concentration of the one or more siRNAs. In another preferred embodiment, none of the siRNAs in the one or more siRNAs constitutes more than 90%, 80%, 70%, 50%, or 20% of the total siRNA concentration of the one or more siRNAs. In a preferred embodiment, the composition of the one or more siRNAs, including the number of different siRNAs and the concentration of each siRNA, is chosen such that the one or more siRNAs causes less than 30%, 20%, 10% or 5%, 1%, 0.1% or 0.01% of silencing of any off-target genes. In other embodiments, each siRNA in the one or more siRNAs has a concentration that is lower than the optimal concentration when used individually. In a preferred embodiment, at least one siRNA in the one or more siRNAs has an concentration that is lower than the concentration of the siRNA that is effective to achieve at least 30%, 50%, 75%, 80%, 85%, 90% or 95% silencing when used in the absence of other siRNAs or in the absence of other siRNAs designed to silence the gene. In another preferred embodiment, each different siRNA in the one or more siRNAs has a concentration that causes less than 30%, 20%, 10% or 5% of silencing of the gene when used in the absence of other siRNAs or in the absence of other siRNAs designed to silence the gene. In a preferred embodiment, each siRNA has a concentration that causes less than 30%, 20%, 10% or 5% of silencing of the target gene when used alone, while the plurality of siRNAs causes at least 80% or 90% of silencing of the target gene.

In one embodiment, each said group of one or more cells is obtained by transfection with said one or more different siRNAs prior to said step of contacting. In another embodiment, the primary target is KSP. In preferred embodiments, said different secondary genes comprises at least 5, at least 10, at least 100, at least 1,000, at least 5,000 different genes. In one embodiment, said different secondary genes are different endogenous genes. In one embodiment, said cell type is a cancer cell type.

In still another aspect, the invention provides a method for treating a mammal having a cancer, comprising administering to said mammal a therapeutically sufficient amount of an agent, said agent regulating the expression of a STK6 or TPX2 gene and/or activity of a protein encoded by said STK6 or TPX2 gene, wherein said mammal is subject to a therapy comprising administering to said mammal a therapeutically sufficient amount of a KSP inhibitor. The invention also provides a method for treating a mammal having a cancer, comprising administering to said mammal i) a therapeutically sufficient amount of an agent, said agent regulating the expression of a STK6 or TPX2 gene and/or activity of a protein encoded by said STK6 or TPX2 gene, and ii) a therapeutically sufficient amount of a KSP inhibitor. In one embodiment, said agent reduces the expression of said STK6 or TPX2 gene in cells of said cancer. In a preferred embodiment, said agent comprises an siRNA targeting said STK6 or TPX2 gene. In another embodiment, the mammal is a human, and the siRNA can be selected from the group consisting of siRNAs described by SEQ ID NO:1, SEQ ID NO:2, SEQ ID NO:3, SEQ ID NO:4, SEQ ID NO:5, and SEQ ID NO:6. In another preferred embodiment, said agent comprises an siRNA targeting said TPX2 gene. In another embodiment, mammal is a human, and the siRNA can be selected from the group consisting of siRNAs described by SEQ ID NO:1237, SEQ ID NO:1238, and SEQ ID NO:1239.

In another embodiment, the invention provides a method for treating a mammal having a cancer, comprising administering to said mammal i) a therapeutically sufficient amount of a first agent, said first agent regulating the expression of a STK6 or TPX2 gene and/or activity of a protein encoded by said STK6 or TPX2 gene, and ii) a therapeutically sufficient amount of a second agent, said second agent regulating the expression of a KSP gene and/or activity of a protein encoded by said KSP gene. In a preferred embodiment, the first agent is an siRNA targeting said STK6 or TPX2 gene, and said second agent comprises an siRNA targeting said KSP gene. In another preferred embodiment, said mammal is a human, and wherein said siRNA is selected from the group consisting of siRNAs described by SEQ ID NO:1, SEQ ID NO:2, SEQ ID NO:3, SEQ ID NO:4, SEQ ID NO:5, and SEQ ID NO:6. In another preferred embodiment, said agent comprises an siRNA targeting said TPX2 gene. In another embodiment, mammal is a human, and the siRNA can be selected from the group consisting of siRNAs described by SEQ ID NO:1237, SEQ ID NO:1238, and SEQ ID NO:1239.

In still another embodiment, the invention provides a method for evaluating resistance of a cell to the growth inhibitory effect of a KSP inhibitor, said method comprising determining an expression level of a STK6 or TPX2 gene in said cell, wherein said expression level above a predetermined threshold level indicates that said cell is resistant to the growth inhibitory effect of said KSP inhibitor. In a preferred embodiment, the expression level of said STK6 or TPX2 gene is determined by a method comprising measuring the expression level of said STK6 or TPX2 gene using one or more polynucleotide probes, each of said one or more polynucleotide probes comprising a nucleotide sequence in said STK6 or TPX2 gene. Said one or more polynucleotide probes can be polynucleotide probes on a microarray.

In still another embodiment, the invention provides a method for evaluating resistance of a cell to the growth inhibitory effect of a KSP inhibitor, said method comprising determining a level of abundance of a protein encoded by a STK6 or TPX2 gene in said cell, wherein said level of abundance of said protein above a predetermined threshold level indicates that said cell is resistant to the growth inhibitory effect of said KSP inhibitor. The invention also provides a method for evaluating resistance of a cell to the growth inhibitory effect of a KSP inhibitor, said method comprising determining a level of activity of a protein encoded by a STK6 or TPX2 gene in said cell, wherein said activity level above a predetermined threshold level indicates that said cell is resistant to the growth inhibitory effect of said KSP inhibitor. In a preferred embodiment, said cell is a human cell.

In still another embodiment, the invention provides a method for regulating resistance of a cell to the growth inhibitory effect of a KSP inhibitor, comprising contacting said cell with a sufficient amount of an agent, said agent regulating the expression of a STK6 or TPX2 gene and/or the activity of a protein encoded by said STK6 or TPX2 gene. The invention also provides a method for regulating resistance of a cell to the growth inhibitory effect of a KSP inhibitor in a mammal, comprising administering to said mammal a therapeutically sufficient amount of an agent, said agent regulating the expression of a STK6 or TPX2 gene and/or the activity of a protein encoded by said STK6 or TPX2 gene. The invention further provides a method for regulating growth of a cell, comprising contacting said cell with i) a sufficient amount of an agent that regulates the expression of a STK6 or TPX2 gene and/or the activity of a protein encoded by said STK6 or TPX2 gene; and ii) a sufficient amount of a KSP inhibitor. Preferably, the agent reduces the expression of said STK6 or TPX2 gene in said cell. In a preferred embodiment, said agent comprises an siRNA targeting said STK 6 gene. In another preferred embodiment, said cell is a human cell, and wherein said siRNA is selected from the group consisting of siRNAs described by SEQ ID NO:1, SEQ ID NO:2, SEQ ID NO:3, SEQ ID NO:4, SEQ ID NO:5, and SEQ ID NO:6. In another preferred embodiment, said agent comprises an siRNA targeting said TPX2 gene. In another embodiment, cell is a human cell, and the siRNA can be selected from the group consisting of siRNAs described by SEQ ID NO:1237, SEQ ID NO:1238, and SEQ ID NO:1239.

In still another embodiment, the invention provides a method of identifying an agent that is capable of regulating resistance of a cell to the growth inhibitory effect of a KSP inhibitor, wherein said agent is capable of modulating the expression of a STK6 or TPX2 gene and/or the activity of a protein encoded by said STK6 or TPX2 gene, said method comprising comparing inhibitory effect of said KSP inhibitor on cells expressing said STK6 or TPX2 gene in the presence of said agent with inhibitory effect of said KSP inhibitor on cells expressing said STK6 or TPX2 gene in the absence of said agent, wherein a difference in said inhibitory effect of said KSP inhibitor identifies said agent as capable of regulating resistance of said cell to the growth inhibitory effect of said KSP inhibitor.

The invention also provides a method of identifying an agent that is capable of regulating resistance of a cell to the growth inhibitory effect of a KSP inhibitor, wherein said agent is capable of modulating the expression of a STK6 or TPX2 gene and/or activity of a protein encoded by said STK6 or TPX2 gene, said method comprising: (a) contacting a first cell expressing said STK6 or TPX2 gene with said KSP inhibitor in the presence of said agent and measuring a first growth inhibitory effect; (b) contacting a second cell expressing said STK6 or TPX2 gene with said KSP inhibitor in the absence of said agent and measuring a second growth inhibitory effect; and (c) comparing said first and second inhibitory effects measured in said step (a) and (b), wherein a difference between said first and second inhibitory effects identifies said agent as capable of regulating resistance of a cell to the growth inhibitory effect of said KSP inhibitor. In a preferred embodiment, said agent is a molecule which reduces expression of said STK6 or TPX2 gene. In another preferred embodiment, said agent comprises an siRNA targeting said STK 6 gene. In still another preferred embodiment, said cell is a human cell, and wherein said siRNA is selected from the group consisting of siRNAs described by SEQ ID NO:1, SEQ ID NO:2, SEQ ID NO:3, SEQ ID NO:4, SEQ ID NO:5, and SEQ ID NO:6. In another preferred embodiment, said agent comprises an siRNA targeting said TPX2 gene. In another embodiment, the cell is a human cell, and the siRNA can be selected from the group consisting of siRNAs described by SEQ ID NO: SEQ ID NO:1237, SEQ ID NO:1238, and SEQ ID NO:1239.

In still another aspect, the invention provides a cell comprising one or more different small interfering RNAs (siRNAs) targeting a STK6 or TPX2 gene in said cell. The cell can be a human cell. The cell can also be a murine cell. In one embodiment, said cell is a human cell, and each of said one or more different siRNA is selected from the group consisting of siRNAs described by SEQ ID NO:1, SEQ ID NO:2, SEQ ID NO:3, SEQ ID NO:4, SEQ ID NO:5, and SEQ ID NO:6. In another embodiment, the cell is a human cell, and the siRNA can be selected from the group consisting of siRNAs described by SEQ ID NO:1237, SEQ ID NO:1238, and SEQ ID NO:1239. In one embodiment, said cell is produced by transfection using a composition of said one or more different siRNAs, wherein the total siRNA concentration of said composition is an optimal concentration for silencing said STK6 or TPX2 gene, wherein said optimal concentration is a concentration further increase of which does not increase the level of silencing substantially. In one embodiment, said optimal concentration is a concentration further increase of which does not increase the level of silencing by more than 20%, more than 10%, or more than 5%. In one embodiment, the concentration of each said different siRNA is about the same. In one embodiment, the respective concentrations of said different siRNAs are different from each other by less than 50%, less than 20%, or less than 10%. In another embodiment, none of the siRNAs in said composition has a concentration that is more than 80%, more than 50%, or more than 20% of said total siRNA concentration of said different siRNAs. In another embodiment, at least one siRNA in said composition has a concentration that is more than 20% or more than 50% of said total siRNA concentration of said different siRNAs. In another embodiment, the number of different siRNAs and the concentration of each siRNA in said composition is chosen such that said composition causes less than 10%, less than 1%, less than 0.1%, or less than 0.01% of silencing of any off-target genes.

In still another aspect, the invention provides a microarray for diagnosing resistance of a cell to the growth inhibitory effect of a KSP inhibitor. The microarray comprising one or more polynucleotide probes, wherein each said polynucleotide probe comprises a nucleotide sequence in a STK6 or TPX2 gene.

In still another aspect, the invention provides kit for diagnosis of resistance of a cell to the growth inhibitory effect of a KSP inhibitor. The kit comprises in one or more containers one or more polynucleotide probes, wherein each said polynucleotide probe comprises a nucleotide sequence in a STK6 or TPX2 gene. The invention also provides a kit for screening for agents which regulate resistance of a cell to the growth inhibitory effect of a KSP inhibitor. The kit comprises in one or more containers (i) a cell comprising one or more different small interfering RNAs (siRNAs) targeting a STK6 or TPX2 gene in said cell; and (ii) a KSP inhibitor. In still another aspect, the invention provides a kit for treating a mammal having a cancer, which comprises in one or more containers (i) a sufficient amount of an agent that regulates the expression of a STK6 or TPX2 gene and/or the activity of a protein encoded by said STK6 or TPX2 gene; and (ii) a KSP inhibitor.

In the invention, the KSP inhibitor can be (1S)-1-{[(2S)-4-(2,5-difluorophenyl)-2-phenyl-2,5-dihydro-1H-pyrrol-1-yl]carbonyl}-2-methylpropylamine as described in PCT application PCT/US03/18482, filed Jun. 12, 2003.

The invention also provides a method for identifying a gene which interacts with a primary target gene in a cell of a cell type. The method comprises (a) contacting one or more cells of said cell type with an agent, wherein said agent modulates the expression of a secondary target gene and/or the activity of a protein encoded by said secondary target gene, and wherein said one or more cells express a first small interference RNA (siRNA) targeting said primary target gene; (b) comparing the effect of said agent on said one or more cells of said clone to the effect of said agent on a cell of said cell type not expressing said first siRNA; and (c) identifying said secondary target gene as a gene that interacts with said primary target gene in a cell of said cell type if the effect of said agent on said one or more cells expressing said first siRNA is different as compared to the effect of said agent on a cell of said cell type not expressing said first siRNA.

In a specific embodiment, the method comprises (a) generating a clone of cells of said cell type which express a first small interference RNA (siRNA) targeting said primary target gene; (b) contacting one or more cells of said clone with an agent, wherein said agent modulates the expression of a secondary target gene and/or the activity of a protein encoded by said secondary target gene; (c) comparing the effect of said agent on said one or more cells of said clone to the effect of said agent on a cell of said cell type not expressing said first siRNA; and (d) identifying said secondary target gene as a gene that interacts with said primary target gene in a cell of said cell type if the effect of said agent on said one or more cells expressing said first siRNA is different as compared to the effect of said agent on a cell of said cell type not expressing said first siRNA.

In some embodiments, the effect of said agent on said one or more cells expressing said first siRNA is enhanced as compared to the effect of said agent on a cell of said cell type not expressing said first siRNA. In some other embodiments, the effect of said agent on said one or more cells expressing said first siRNA is reduced as compared to the effect of said agent on a cell of said cell type not expressing said first siRNA. In one embodiment, said agent is an inhibitor of said secondary target gene. The effect of said agent can be a change in the sensitivity of cells of said cell type to a drug, e.g., to a DNA damaging agent, e.g., a topoisomerase I inhibitor, e.g., camptothecin, a topoisomerase II inhibitor, e.g., doxorubicin, a DNA binding agent, e.g., cisplatin, an anti-metabolite, or ionizing radiation.

In another embodiment, said agent comprises one or more second siRNAs targeting and silencing said secondary target gene. Preferably, said one or more second siRNAs comprises at least k different siRNAs, e.g., at least 2, 3, 4, 5, 6 and 10 different siRNAs. In a preferred embodiment, the total siRNA concentration of the one or more second siRNAs is about the same as the concentration of a single siRNA when used individually, e.g., 100 nM. Preferably, the total concentration of the one or more second siRNAs is an optimal concentration for silencing the intended secondary target gene. An optimal concentration is a concentration further increase of which does not increase the level of silencing substantially. In one embodiment, the optimal concentration is a concentration further increase of which does not increase the level of silencing by more than 5%, 10% or 20%. In a preferred embodiment, the one or more second siRNAs comprise each siRNA in equal proportion. In another preferred embodiment, the one or more second siRNAs comprise each siRNA in proportions different from each other by less than 5%, 10%, 20% or 50%. In a preferred embodiment, at least one of the one or more second siRNAs constitutes more than 90%, 80%, 70%, 50%, or 20% of the total siRNA concentration of the one or more second siRNAs. In another preferred embodiment, none of the siRNAs in the one or more second siRNAs constitutes more than 90%, 80%, 70%, 50%, or 20% of the total siRNA concentration of the one or more second siRNAs. In a preferred embodiment, the composition of the one or more second siRNAs, including the number of different siRNAs and the concentration of each siRNA, is chosen such that the one or more second siRNAs causes less than 30%, 20%, 10% or 5%, 1%, 0.1% or 0.01% of silencing of any off-target genes. In other embodiments, each siRNA in the one or more second siRNAs has a concentration that is lower than the optimal concentration when used individually. In a preferred embodiment, at least one siRNA in the one or more second siRNAs has an concentration that is lower than the concentration of the siRNA that is effective to achieve at least 30%, 50%, 75%, 80%, 85%, 90% or 95% silencing when used in the absence of other siRNAs or in the absence of other siRNAs designed to silence the gene. In another preferred embodiment, each different siRNA in the one or more second siRNAs has a concentration that causes less than 30%, 20%, 10% or 5% of silencing of the gene when used in the absence of other siRNAs or in the absence of other siRNAs designed to silence the gene. In a preferred embodiment, each siRNA has a concentration that causes less than 30%, 20%, 10% or 5% of silencing of the secondary target gene when used alone, while the plurality of siRNAs causes at least 80% or 90% of silencing of the secondary target gene.

In one embodiment, said cell type is a cancer cell type. In another embodiment, said primary target gene is p53.

In a preferred embodiment, steps (b)-(d) of the method are repeated for each of a plurality of different secondary target genes. The plurality of secondary target genes can comprise at least 5, 10, 100, 1,000, and 5,000 different genes.

The invention also provides a method for treating a mammal having a cancer. The method comprises administering to said mammal a therapeutically sufficient amount of an agent, said agent regulating the expression of a gene and/or activity of a protein encoded by said gene, wherein said mammal is subject to a therapy comprising administering to said mammal a therapeutically sufficient amount of a composition comprising one or more DNA damaging agents. In one embodiment, the invention provides a method for treating a mammal having a cancer, comprising administering to said mammal i) a therapeutically sufficient amount of an agent, said agent regulating the expression of a gene and/or activity of a protein encoded by said gene, and ii) a therapeutically sufficient amount of a composition comprising one or more DNA damaging agents.

Preferably, said agent reduces the expression of said gene in cells of said cancer. In a preferred embodiment, said agent comprises an siRNA targeting said gene. In specific embodiment, said gene is EPHB3, WEE1, ELK1, STK6, CHEK1 or BRCA2. The agent can also be an agent that enhances the expression of said gene in cells of said cancer. The one or more DNA damaging agents can comprise a topoisomerase I inhibitor, e.g., camptothecin, a topoisomerase II inhibitor, e.g., doxorubicin, a DNA binding agent, e.g., cisplatin, an anti-metabolite, or ionizing radiation.

The invention also provides a method for evaluating sensitivity of a cell to the growth inhibitory effect of a DNA damaging agent. The method comprises determining a transcript level of a gene in said cell, wherein said transcript level below a predetermined threshold level indicates that said cell is sensitive to the growth inhibitory effect of said DNA damaging agent. The DNA damaging agent can be a topoisomerase I inhibitor, e.g., camptothecin, a topoisomerase II inhibitor, e.g., doxorubicin, a DNA binding agent, e.g., cisplatin, an anti-metabolite, or ionizing radiation. In a preferred embodiment, said gene is EPHB3, WEE1, ELK1, STK6, CHEK1 or BRCA2. In a preferred embodiment, said transcript level of said gene is determined by a method comprising measuring the transcript level of said gene using one or more polynucleotide probes, each of said one or more polynucleotide probes comprising a nucleotide sequence in said gene. In one embodiment, said one or more polynucleotide probes are polynucleotide probes on a microarray.

In another embodiment, the invention provides a method for evaluating sensitivity of a cell, e.g., a human cell, to the growth inhibitory effect of a DNA damaging agent. The method comprises determining a level of abundance of a protein encoded by a gene in said cell, wherein said level of abundance of said protein below a predetermined threshold level indicates that said cell is sensitive to the growth inhibitory effect of said DNA damaging agent. The invention also provides a method for evaluating sensitivity of a cell, e.g., a human cell, to the growth inhibitory effect of a DNA damaging agent, said method comprising determining a level of activity of a protein encoded by a gene in said cell, wherein said activity level above a predetermined threshold level indicates that said cell is sensitive to the growth inhibitory effect of said DNA damaging agent. The DNA damaging agent can be a topoisomerase I inhibitor, e.g., camptothecin, a topoisomerase II inhibitor, e.g., doxorubicin, a DNA binding agent, e.g., cisplatin, an anti-metabolite, or ionizing radiation. In a preferred embodiment, said gene is EPHB3, WEE1, ELK1, STK6, CHEK1 or BRCA2.

The invention also provides a method for regulating sensitivity of a cell to DNA damage. The method comprises contacting said cell with a sufficient amount of an agent, said agent regulating the expression of a gene selected from the group consisting of EPHB3, WEE1, ELK1, STK6, BRCA1, BRCA2, BARD1, and RAD51 and/or the activity of a protein encoded by said gene. The invention also provides a method for regulating growth of a cell, comprising contacting said cell with i) a sufficient amount of an agent that regulates the expression of a gene selected from the group consisting of EPHB33, WEE1, ELK1, STK6, BRCA1, BRCA2, BARD1, and RAD51 and/or the activity of a protein encoded by said gene; and ii) a sufficient amount of a DNA damaging agent. The DNA damaging agent can be a topoisomerase I inhibitor, e.g., camptothecin, a topoisomerase II inhibitor, e.g., doxorubicin, a DNA binding agent, e.g., cisplatin, an anti-metabolite, or ionizing radiation.

In one embodiment, said agent reduces the expression of said gene in said cell. In a preferred embodiment, said agent comprises an siRNA targeting said gene. In another preferred embodiment, said agent comprises 2, 3, 4, 5, 6, or 10 different siRNAs targeting said gene. In a preferred embodiment, the total siRNA concentration of the different siRNAs targeting said is about the same as the concentration of a single siRNA when used individually, e.g., 100 nM. Preferably, the total concentration of the different siRNAs targeting said gene is an optimal concentration for silencing the gene. An optimal concentration is a concentration further increase of which does not increase the level of silencing substantially. In one embodiment, the optimal concentration is a concentration further increase of which does not increase the level of silencing by more than 5%, 10% or 20%. In a preferred embodiment, the different siRNAs comprise each siRNA in equal proportion. In another preferred embodiment, the different siRNAs comprise each siRNA in proportions different from each other by less than 5%, 10%, 20% or 50%. In a preferred embodiment, at least one of the different siRNAs constitutes more than 90%, 80%, 70%, 50%, or 20% of the total siRNA concentration of the different siRNAs. In another preferred embodiment, none of the siRNAs in the different siRNAs constitutes more than 90%, 80%, 70%, 50%, or 20% of the total siRNA concentration of the different siRNAs. In a preferred embodiment, the composition of the different siRNAs, including the number of different siRNAs and the concentration of each siRNA, is chosen such that the different siRNAs causes less than 30%, 20%, 10% or 5%, 1%, 0.1% or 0.01% of silencing of any off-target genes. In other embodiments, each siRNA in the different siRNAs has a concentration that is lower than the optimal concentration when used individually. In a preferred embodiment, at least one siRNA in the different siRNAs has an concentration that is lower than the concentration of the siRNA that is effective to achieve at least 30%, 50%, 75%, 80%, 85%, 90% or 95% silencing when used in the absence of other siRNAs or in the absence of other siRNAs designed to silence the gene. In another preferred embodiment, each different siRNA has a concentration that causes less than 30%, 20%, 10% or 5% of silencing of the gene when used in the absence of other siRNAs or in the absence of other siRNAs designed to silence the gene. In a preferred embodiment, each siRNA has a concentration that causes less than 30%, 20%, 10% or 5% of silencing of the target gene when used alone, while the plurality of siRNAs causes at least 80% or 90% of silencing of the target gene.

The invention also provides a method of identifying an agent that is capable of regulating sensitivity of a cell to the growth inhibitory effect of a DNA damaging agent, wherein said agent is capable of modulating the expression of a gene selected from the group consisting of EPHB3, WEE1, ELK1, STK6, BRCA1, BRCA2, BARD1, and RAD51 and/or the activity of a protein encoded by said gene, said method comprising comparing inhibitory effect of said DNA damaging agent on cells expressing said gene in the presence of said agent with inhibitory effect of said DNA damaging agent on cells expressing said gene in the absence of said agent, wherein a difference in said inhibitory effect of said DNA damaging agent identifies said agent as capable of regulating sensitivity of said cell to the growth inhibitory effect of said DNA damaging agent. In one embodiment, the invention provides a method of identifying an agent that is capable of regulating sensitivity of a cell to the growth inhibitory effect of a DNA damaging agent, wherein said agent is capable of modulating the expression of a gene selected from the group consisting of EPHB3, WEE1, ELK1, STK6, BRCA1, BRCA2, BARD1, and RAD51 and/or activity of a protein encoded by said gene, said method comprising: (a) contacting a first cell expressing said gene with said DNA damaging agent in the presence of said agent and measuring a first growth inhibitory effect; (b) contacting a second cell expressing said gene with said DNA damaging agent in the absence of said agent and measuring a second growth inhibitory effect; and (c) comparing said first and second inhibitory effects measured in said step (a) and (b), wherein a difference between said first and second inhibitory effects identifies said agent as capable of regulating sensitivity of a cell to the growth inhibitory effect of said DNA damaging agent.

Preferably, said cell expresses an siRNA targeting a primary target gene. In one embodiment, said primary target gene is p53.

In a preferred embodiment, said agent is a molecule that reduces expression of said gene. In one embodiment, said agent comprises an siRNA targeting said gene. In another embodiment, said agent comprises 2, 3, 4, 5, 6, or 10 different siRNAs targeting said gene. In a preferred embodiment, the total siRNA concentration of the different siRNAs targeting said is about the same as the concentration of a single siRNA when used individually, e.g., 100 nM. Preferably, the total concentration of the different siRNAs targeting said gene is an optimal concentration for silencing the gene. An optimal concentration is a concentration further increase of which does not increase the level of silencing substantially. In one embodiment, the optimal concentration is a concentration further increase of which does not increase the level of silencing by more than 5%, 10% or 20%. In a preferred embodiment, the different siRNAs comprise each siRNA in equal proportion. In another preferred embodiment, the different siRNAs comprise each siRNA in proportions different from each other by less than 5%, 10%, 20% or 50%. In a preferred embodiment, at least one of the different siRNAs constitutes more than 90%, 80%, 70%, 50%, or 20% of the total siRNA concentration of the different siRNAs. In another preferred embodiment, none of the siRNAs in the different siRNAs constitutes more than 90%, 80%, 70%, 50%, or 20% of the total siRNA concentration of the different siRNAs. In a preferred embodiment, the composition of the different siRNAs, including the number of different siRNAs and the concentration of each siRNA, is chosen such that the different siRNAs causes less than 30%, 20%, 10% or 5%, 1%, 0.1% or 0.01% of silencing of any off-target genes. In other embodiments, each siRNA in the different siRNAs has a concentration that is lower than the optimal concentration when used individually. In a preferred embodiment, at least one siRNA in the different siRNAs has an concentration that is lower than the concentration of the siRNA that is effective to achieve at least 30%, 50%, 75%, 80%, 85%, 90% or 95% silencing when used in the absence of other siRNAs or in the absence of other siRNAs designed to silence the gene. In another preferred embodiment, each different siRNA has a concentration that causes less than 30%, 20%, 10% or 5% of silencing of the gene when used in the absence of other siRNAs or in the absence of other siRNAs designed to silence the gene. In a preferred embodiment, each siRNA has a concentration that causes less than 30%, 20%, 10% or 5% of silencing of the target gene when used alone, while the plurality of siRNAs causes at least 80% or 90% of silencing of the target gene.

In the method, said DNA damaging agent can be a topoisomerase I inhibitor, e.g., camptothecin, a topoisomerase II inhibitor, e.g., doxorubicin, a DNA binding agent, e.g., cisplatin, an anti-metabolite, or an ionizing radiation.

The invention also provides a cell comprising one or more different small interfering RNAs (siRNAs) targeting a gene selected from the group consisting of EPHB3, WEE1, ELK1, BRCA1, BRCA2, BARD1, and RAD51 in said cell. In one embodiment, said one or more different siRNAs comprises 2, 3, 4, 5, 6, or 10 different siRNAs. In a preferred embodiment, the total siRNA concentration of the one or more different siRNAs is about the same as the concentration of a single siRNA when used individually, e.g., 100 nM. Preferably, the total concentration of the one or more siRNAs is an optimal concentration for silencing the intended target gene. An optimal concentration is a concentration further increase of which does not increase the level of silencing substantially. In one embodiment, the optimal concentration is a concentration further increase of which does not increase the level of silencing by more than 5%, 10% or 20%. In a preferred embodiment, the one or more siRNAs comprise each siRNA in equal proportion. In another preferred embodiment, the one or more siRNAs comprise each siRNA in proportions different from each other by less than 5%, 10%, 20% or 50%. In a preferred embodiment, at least one of the one or more siRNAs constitutes more than 90%, 80%, 70%, 50%, or 20% of the total siRNA concentration of the one or more siRNAs. In another preferred embodiment, none of the siRNAs in the one or more siRNAs constitutes more than 90%, 80%, 70%, 50%, or 20% of the total siRNA concentration of the one or more siRNAs. In a preferred embodiment, the composition of the one or more siRNAs, including the number of different siRNAs and the concentration of each siRNA, is chosen such that the one or more siRNAs causes less than 30%, 20%, 10% or 5%, 1%, 0.1% or 0.01% of silencing of any off-target genes. In other embodiments, each siRNA in the one or more siRNAs has a concentration that is lower than the optimal concentration when used individually. In a preferred embodiment, at least one siRNA in the one or more siRNAs has an concentration that is lower than the concentration of the siRNA that is effective to achieve at least 30%, 50%, 75%, 80%, 85%, 90% or 95% silencing when used in the absence of other siRNAs or in the absence of other siRNAs designed to silence the gene. In another preferred embodiment, each different siRNA in the one or more siRNAs has a concentration that causes less than 30%, 20%, 10% or 5% of silencing of the gene when used in the absence of other siRNAs or in the absence of other siRNAs designed to silence the gene. In a preferred embodiment, each siRNA has a concentration that causes less than 30%, 20%, 10% or 5% of silencing of the target gene when used alone, while the plurality of siRNAs causes at least 80% or 90% of silencing of the target gene.

The invention also provides a microarray for diagnosing sensitivity of a cell to the growth inhibitory effect of a DNA damaging agent. The microarray comprises one or more polynucleotide probes, wherein each said polynucleotide probe comprises a nucleotide sequence in one or more genes selected from the group consisting of EPHB3, WEE1, ELK1, STK6, BRCA1, BRCA2, BARD1, and RAD51.

The invention also provides a kit for diagnosis of sensitivity of a cell to the growth inhibitory effect of a DNA damaging agent. The kit comprises in one or more containers one or more polynucleotide probes, wherein each said polynucleotide probe comprises a nucleotide sequence in a gene selected from the group consisting of EPHB3, WEE1, ELK1, STK6, BRCA1, BRCA2, BARD1, and RAD51.

The invention also provides a kit for screening for agents which regulate sensitivity of a cell to the growth inhibitory effect of a DNA damaging agent. The kit comprises in one or more containers (i) a cell comprising one or more different small interfering RNAs (siRNAs) targeting a gene selected from the group consisting of EPHB3, WEE1, ELK1, BRCA1, BRCA2, BARD1, and RAD51 in said cell; and (ii) said DNA damaging agent.

The invention also provides a kit for treating a mammal having a cancer, comprising in one or more containers (i) a sufficient amount of an agent that regulates the expression of a gene selected from the group consisting of EPHB3, WEE1, ELK1, STK6, BRCA1, BRCA2, BARD1, and RAD51 and/or the activity of a protein encoded by said gene; and (ii) a DNA damaging agent.

In the kit of the invention, the DNA damaging agent can be a topoisomerase I inhibitor, e.g., camptothecin, a topoisomerase II inhibitor, e.g., doxorubicin, a DNA binding agent, e.g., cisplatin, or an anti-metabolite.

The invention also provides a method of evaluating the responsiveness of cells of a cell type to treatment of a drug, comprising (a) contacting one or more cells of said cell type with said drug, wherein said one or more cells express a first small interference RNA (siRNA) targeting a primary target gene, and wherein said one or more cells are subject to treatment of a composition that modulates the expression of one or more secondary target genes and/or the activity of one or more proteins encoded respectively by said one or more secondary target genes; (b) contacting one or more cells of said cell type with said drug, wherein said one or more cells do not express a small interference RNA (siRNA) targeting said primary target gene, and wherein said one or more cells are subject to treatment of said agent that modulates the expression of a secondary target gene and/or the activity of a protein encoded by said secondary target gene; and (c) comparing the effect of said drug on said one or more cells measured in step (a) to the effect of said drug on said one or more cells measured in step (b), thereby evaluating the responsiveness of said cells to treatment of said drug. In one embodiment, the method further comprises a step (d) repeating steps (a)-(b) for each of a plurality of different secondary target genes.

In a specific embodiment, the invention provides a method for evaluating the responsiveness of cells of a cell type to treatment of a drug, said method comprising (a) generating a clone of cells of said cell type which express a first small interference RNA (siRNA) targeting a primary target gene; (b) contacting one or more cells of said clone which express said first siRNA with said drug, wherein said one or more cells are subject to treatment of an agent that modulates the expression of a secondary target gene and/or the activity of a protein encoded by said secondary target gene; (c) contacting one or more cells of said cell type which do not express a small interference RNA (siRNA) targeting said primary target gene with said drug, wherein said one or more cells are subject to treatment of said agent that modulates the expression of a secondary target gene and/or the activity of a protein encoded by said secondary target gene; and (d) comparing the effect of said drug on said one or more cells measured in step (b) to the effect of said drug on said one or more cells measured in step (c), thereby evaluating the responsiveness of said cells to treatment of said drug. In one embodiment, the method further comprises a step (e) repeating steps (b)-(d) for each of a plurality of different secondary target genes.

In one embodiment, the effect of said drug on said one or more cells expressing said first siRNA is enhanced as compared to the effect of said drug on a cell of said cell type not expressing said first siRNA. In another embodiment, the effect of said drug on said one or more cells expressing said first siRNA is reduced as compared to the effect of said drug on a cell of said cell type not expressing said first siRNA.

In one embodiment, said composition comprises one or more inhibitors of said one or more secondary target gene. In a preferred embodiment, said composition comprises one or more second siRNAs targeting and silencing said one or more secondary target gene.

In one embodiment, said one or more second siRNAs comprises at least k different siRNAs, wherein said k is selected from the group consisting of 2, 3, 4, 5, 6 and 10. In one embodiment, the total siRNA concentration of said at least k different siRNAs in said agent is an optimal concentration for silencing said secondary target gene, wherein said optimal concentration is a concentration further increase of which does not increase the level of silencing substantially. In one embodiment, said optimal concentration is a concentration further increase of which does not increase the level of silencing by more than 20%, more than 10%, or more than 5%. In another embodiment, the concentration of each said at least k different siRNA is about the same. In another embodiment, the respective concentrations of said at least k different siRNAs are different from each other by less than 50%, less than 20%, or less than 10%. In still another embodiment, none of the siRNAs in said agent has a concentration that is more than 80%, more than 50%, or more than 20% of said total siRNA concentration of said different siRNAs. In still another embodiment, at least one siRNA in said agent has a concentration that is more than 20% or more than 50% of said total siRNA concentration of said at least k different siRNAs. In still another embodiment, the number of different siRNAs and the concentration of each siRNA in said agent is chosen such that said agent causes less than 10%, less than 1%, less than 0.1%, or less than 0.01% of silencing of any off-target genes.

In some embodiment, said cell type is a cancer cell type, and said primary target gene is p53. In preferred embodiment, said plurality of secondary target genes comprises at least the number of different genes selected from the group consisting of 5, 10, 100, 1,000, and 5,000 different genes.

In one embodiment, said drug is a DNA damaging agent, e.g., a DNA damaging agent selected from the group consisting of topoisomerase I inhibitor, topoisomerase II inhibitor, DNA binding agent, and ionizing radiation. In a specific embodiment, said DNA damaging agent is selected from the group consisting of doxorubicin, camptothecin, and cisplatin.

### 4. BRIEF DESCRIPTION OF FIGURES

FIG. 1 shows correlation between mRNA silencing and growth inhibition phenotype for STK6. HeLa cells were transfected with six individual siRNAs to STK6. At 24 hrs post transfection, one set of cells was harvested for RNA isolation and determination of STK6 mRNA levels by TaqMan analysis using an Assay on Demand (Applied Biosciences). Another set of cells was incubated further (72 hrs total) and cellular growth was assessed in triplicate wells using an Alamar Blue assay. Values for mRNA levels (X axis) and cell growth (Y axis) for each were normalized to a mock transfected control. For TaqMan analysis, each data point represents a single RNA sample assayed in triplicate (and normalized to GUS); variation between replicates was generally <10%. For growth assay determinations, each data point represents the average of triplicate determinations that generally varied from the mean by <20%. The solid line represents an ideal 1:1 relationship between silencing and phenotype.

FIG. 2 shows synthetic lethal interactions between STK6 and KSP. HeLa cells were transfected with increasing concentrations of siRNA to luciferase (negative control) and STK6 (top panel) or PTEN (bottom panel) and tested for growth relative to control (luciferase-treated) in the three-day Alamar Blue assay. Where indicated, cells were also treated with 25 nM KSPi, (1S)-1-{[(2S)-4-(2,5-difluorophenyl)-2-phenyl-2,5-dihydro-1H-pyrrol-1-yl]carbonyl}-2-methylpropylamine; the EC50 for HeLa cells assayed under these conditions was ˜80 nM. Shown are the mean±SD (error bars) of triplicate determinations.

FIG. 3 demonstrates that stable expression of a TP53 shRNA effectively silences the target gene. HCT116 cells were transfected with a TP53-targeting shRNA plasmid (pRS-p53). Shown are the TP53 mRNA levels in wild type (WT) cells and in two independent clones (A5 and A11) of cells stably transfected with pRS-p53. TP53 mRNA levels were silenced >95% in clones A5 and A11 (Middle bars). Transient introduction of the pRS-p53 into HCT116 cells achieves ˜80% silencing 24 hr post transfection (Right bar).

FIG. 4 shows maintenance of mRNA silencing by stable shRNA expression following siRNA supertransfection. (A) pRS-p53 does not affect CHEK1 silencing by siRNAs and vice versa. A pool of three siRNAs targeting CHEK1 was transiently transfected into WT and pRS-p53 stably transfected HCT116 cells (clone A11). CHEK1 and TP53 mRNA levels were measured by Taqman analysis (left and right panels, respectively). (B) Supertransfected KNSL1 siRNAs do not competitively inhibit silencing by pRS-STK6. STK6 and KNSL1 siRNAs were transiently co-transfected into WT SW480 cells and KNSL1 siRNAs were supertransfected into pRS-STK6 stably transfected SW480 cells. STK6 mRNA levels were measured by Taqman analysis. For the left set of bars, STK6 siRNA (10 nM) was used alone or together with one of three different individual KNSL1 siRNAs (10 nM each). The KNSL1 siRNAs variably inhibit silencing by STK6 siRNAs. For the right two sets of bars, KNSL1 siRNAs were used as competitors at 10 or 100 nM against the stably expressed STK6 shRNA.

FIG. 5 demonstrates that siRNA library screens in the absence of DNA damage show good correlation between cells with and without a shRNA targeting p53. (x axis) pRS (vector alone) cells were supertransfected with pools of three siRNAs each targeting one of 800 genes and tested for growth related phenotypes; (y axis) pRS-p53 cells assayed in the same manner. The tight correlation between the two sets of data indicates that the performance of the siRNA pools is likely not affected by the presence of the shRNA suggesting that the shRNA does not compete with the siRNAs.

FIG. 6 shows that CHEK1 silencing decreases G2 checkpoint arrest in pRS-p53 cells. A549 cells stably transfected with vector only (pRS) or pRS-p53 cells were supertransfected with control (luc, luciferase) siRNA or with a pool of three siRNAs to CHEK1. Doxorubicin (200 ng/ml) was added 24 hr post-transfection and cell cycle profiles were analyzed 48 hr after doxorubicin addition. TP53 mRNA levels in pRS-p53 cells was reduced ˜90% compared with pRS cells.

FIG. 7 illustrates the identification of genes that sensitize to Cisplatin. HeLa cells grown in 384 well plates were transfected with siRNA pools representing ˜800 human genes (3 siRNAs/gene, total siRNA concentration 100 nM). Four hours post-transfection, cells were treated with either medium alone (or plus vehicle) (− drug) or medium plus an EC10 concentration of Cisplatin (Cis, + drug). Cell growth was then measured 72 hrs post-transfection using an Alamar Blue assay and is expressed as % growth measured in wells transfected with luciferase siRNA. Each point represents the average of 2-4 replicate determinations.

FIG. 8 shows a comparison of genes that sensitize to different drug treatments. HeLa cells were transfected with siRNAs as shown in FIG. 1 and treated with either medium alone (or plus vehicle), or medium plus an EC10 concentration of Cis, Doxorubicin (Dox) or Camoptothecin (Campto). Cell growth was measured and is expressed the ratio of growth—drug/growth+drug. Dotted red lines indicate two-fold sensitization. Selected genes are indicated.

FIGS. 9A-9C show that silencing of WEE1 sensitizes HeLa cells to DNA damage induced by Dox, Campto, and Cis. FIGS. 9D-91 show that silencing of WEE1 sensitizes p53−A549 cells to DNA damage induced by Dox, Campto, and Cis, but does not sensitize p53+A549 cells to such DNA damage.

FIGS. 10A-10C show that silencing of EPHB3 sensitizes HeLa cells and p53−A549 C7, and to a lesser extent p53+ A549 pRS cells, to DNA damage induced by Dox, Campto, and Cis.

FIGS. 11A-11C show that silencing of STK6 sensitizes HeLa cells and p53−A549 C7, and to a lesser extent p53+ A549 pRS cells to DNA damage induced by Dox, Campto, and Cis.

FIGS. 12A-12C show that silencing of BRCA1 sensitizes HeLa cells and p53−A549 C7 cells to DNA damage induced by Dox, Campto, and Cis. Silencing of BRCA also sensitizes p53+ A549 pRS cells to DNA damage induced by Cis to a lesser extent, but does not sensitize p53+ A549 pRS cells to DNA damage induced by Dox and Campto.

FIGS. 13A-13B show that silencing of BRCA2 sensitizes HeLa cells and p53−A549 C7 cells to DNA damage induced by Dox, Campto, and Cis. FIG. 13C shows that silencing of BRCA2 sensitize p53+ A549 pRS cells to DNA damage induced by Cis to a lesser extent, but not dox and Campto.

FIGS. 14A-14B show that silencing of CHUK sensitizes HeLa cells to DNA damage induced by Dox, Campto, and Cis. FIG. 14C shows that silencing of CHUK sensitizes p53−A549 C7 cells to DNA damage induced by Campto and Cis. FIG. 14D shows that silencing of CHUK does not sensitize p53+ A549 pRS cells to DNA damage induced by Campto and Cis.

FIGS. 15A-C shows results of CHEK1 silencing on the sensitivity of cells to DNA damage. 15A CHEK1 silencing/inhibition sensitizes HeLa cells to DNA damage. 15B CHEK1 silencing/inhibition sensitizes p53−A549 cells. 15C CHEK1 silencing does not sensitize HREP cells to Doxorubicin.

FIG. 16 shows that siRNA mediated knockdown of PLK gene results in a cell cycle arrest and apoptosis.

FIG. 17 shows results of screens for genes that sensitize to KSPi.

FIG. 18 shows results of screens for genes that sensitize to Taxol.

FIG. 19 BRCA complexes enhance cisplatin activity. HeLa cells were transfected in 384 well format with siRNAs pools to ˜2,000 genes (3 siRNAs/gene) and then treated with (Y axis) or without (X axis) cisplatin. Two different cisplatin concentrations were tested, 100 ng/ml (˜EC10, left panel) or 400 ng/ml (˜EC50, right panel). Cell growth was measured 72 hrs post transfection using an Alamar Blue assay. Diagonal lines indicate concordance between the two treatments (black lines), or 2- and 3-fold sensitization by cisplatin treatment (magenta and red lines, respectively).

FIG. 20 Silencing of BRCA1 preferentially sensitizes TP53− cells to DNA damage. A549 cells stably transfected with empty vector (pRS, left panel) or an shRNA targeting TP53 (pRS-TP53, right panel) were supertransfected with siRNAs to luciferase, BRCA1, or BRCA2 prior to treatment with the DNA damaging agent, cisplatin. Cell growth was measured 72 hrs post-transfection using Alamar Blue.

FIG. 21 Silencing of BRCA1 selectively sensitizes TP53-cells to DNA damage. Matched TP53-negative (left column) or positive (right column) A549 cells were transfected with an siRNA to luciferase (top row) or BRCA1 (bottom row) prior to treatment with the DNA damaging agent, bleomycin. Seventy-two hours after transfection, cells were fixed, stained with propidium iodide and analyzed for cell cycle distribution by flow cytometry. The relative fluorescence of cells having 2N or 4N DNA content is indicated with arrows. The gates labeled in red indicate the number of sub-G1 (dead) cells.

FIG. 22 shows results that demonstrate that RAD51/Doxorubicin synergy is greater in TP53-cells.

### 5. DETAILED DESCRIPTION OF THE INVENTION

The invention provides methods and compositions for identifying interactions, e.g., lethal/synthetic lethal interactions, between a gene or its product and an agent, e.g., a drug, using RNA interference. As used herein, the term “gene product” includes mRNA transcribed from the gene and protein encoded by the gene. The invention also provides methods and compositions for treating cancer utilizing synthetic lethal interactions between STK6 kinase (also known as Aurora A kinase) and KSP (a kinesin-like motor protein, also known as KNSL1 or EG5) inhibitors (KSPi's). In this disclosure, a KSPi (1S)-1-{[(2S)-4-(2,5-difluorophenyl)-2-phenyl-2,5-dihydro-1H-pyrrol-1-yl]carbonyl}-2-methylpropylamine 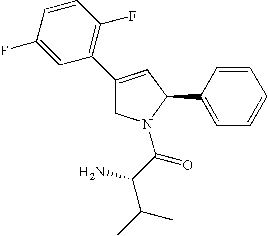
(see, PCT application PCT/US03/18482, filed Jun. 12, 2003, which is incorporated herein by reference in its entirety), is often used. Other KSPi's can also be used in the invention. It is envisioned that methods utilize such other KSPi's are also encompassed by the present invention. The invention also provides methods and compositions for treating cancer utilizing interactions between a DNA damage response gene and a DNA damaging agent.

### 5.1. Methods of Screening of Interaction Using RNA Interference

The invention provides a method of identifying one or more genes in a cell of a cell type which interact with, e.g., modulate the effect of, an agent, e.g., a drug. As used herein, interaction of a gene with an agent or another gene includes interactions of the gene and/or its products with the agent or another gene/gene product. For example, an identified gene may confer resistance or sensitivity to a drug, i.e., reduces or enhances the effect of the drug. Such gene or genes can be identified by knocking down a plurality of different genes in cells of the cell type using a plurality of small interfering RNAs (knockdown cells), each of which targets one of the plurality of different genes, and determining which gene or genes among the plurality of different genes whose knockdown modulates the response of the cell to the agent. In one embodiment, a plurality of different knockdown cells (a knockdown library) are generated, each knockdown cell in the knockdown library comprising a different gene that is knockdown, e.g., by an siRNA. In another embodiment, a plurality of different knockdown cells (a knockdown library) are generated, each knockdown cell in the knockdown library comprising 2 or more different genes that are knockdown, e.g., by shRNA and siRNA targeting different genes. In one embodiment, the knockdown library comprises a plurality of cells, each of which expresses an siRNA targeting a primary gene and is supertransfected with one or more siRNAs targeting a secondary gene. It will be apparent to one skilled in the art that a knockdown cell may also be generated by other means, e.g., by using antisense, ribozyme, antibody, or a small organic or inorganic molecule that target the gene or its product. It is envisioned that any of these other means and means utilizing siRNA can be used alone or in combination to generate a knockdown library of the invention. Any method for siRNA silencing may be used, including methods that allow tuning of the level of silencing of the target gene. Section 5.2., infra, describes various methods that can be used.

In one embodiment, the method of the invention is practiced using an siRNA knockdown library comprising a plurality of cells of a cell type each comprising one of a plurality of siRNAs, each of the plurality of siRNAs targeting and silencing (i.e., knocking down) one of a plurality of different genes in the cell (i.e., knockdown cells). Any known method of introducing siRNAs into a cell can be used for this purpose. Preferably, each of the plurality of cells is generated and maintained separately such that they can be studied separately. Each of the plurality of cells is then treated with an agent, and the effect of the agent on the cell is determined. The effect of the agent on a cell comprising a gene silenced by an siRNA is then compared with the effect of the agent on cells of the cell type which do not comprise an siRNA, i.e., normal cells of the cell type. Knockdown cell or cells which exhibit a change in response to the agent are identified. The gene which is silenced by the comprised siRNA in such a knockdown cell is a gene which modulates the effect of the agent. Preferably, the plurality of siRNAs comprises siRNAs targeting and silencing at least 5, 10, 100, or 1,000 different genes in the cells. In a preferred embodiment, the plurality of siRNAs target and silence endogenous genes.

In a preferred embodiment, the knockdown library comprises a plurality of different knockdown cells having the same gene knocked down, e.g., each cell having a different siRNA targeting and silencing a same gene. The plurality of different knockdown cells having the same gene knocked down can comprises at least 2, 3, 4, 5, 6 or 10 different knockdown cells, each of which comprises an siRNA targeting a different region of the knocked down gene. In another preferred embodiment, the knockdown library comprises a plurality of different knockdown cells, e.g., at least 2, 3, 4, 5, 6, or 10, for each of a plurality of different genes represented in the knockdown library. In still another preferred embodiment, the knockdown library comprises a plurality of different knockdown cells, e.g., at least 2, 3, 4, 5, 6, or 10, for each of all different genes represented in the knockdown library.

In another preferred embodiment, the knockdown library comprises a plurality of different knockdown cells having different genes knocked down, each of the different knockdown cells has two or more different siRNA targeting and silencing a same gene. In preferred embodiment, each different knockdown cell can comprises at least 2, 3, 4, 5, 6 or 10 different siRNAs targeting the same gene at different regions.

In a preferred embodiment, the interaction of a gene with an agent is evaluated based on responses of a plurality of different knockdown cells having the gene knocked down, e.g., each cell having a different siRNA targeting and silencing a same gene. Utilizing the responses of a plurality of different siRNAs allows determination of the on-target and off-target effect of different siRNAs (see, e.g., International application No. PCT/U.S. 2004/015439 by Jackson et al., filed on May 17, 2004).

The effect of the agent on a cell of a cell type may be reduced in a knockdown cell as compared to that of a normal cell of the cell type, i.e., the knockdown of the gene mitigates the effect of the agent. The gene which is knocked down in such a cell is said to confer sensitivity to the agent. Thus, in one embodiment, the method of the invention is used for identifying one or more genes that confer sensitivity to an agent.

The effect of the agent on a cell of a cell type may be enhanced in a knockdown cell as compared to that of a normal cell of the cell type. The gene which is knocked down in such a cell is said to confer resistance to the agent. Thus, in another embodiment, the method of the invention is used for identifying a gene or genes that confers resistance to an agent. The enhancement of an effect of an agent may be additive or synergistic. In one embodiment, the invention provides a method for identifying one or more genes capable of regulating and/or enhancing the growth inhibitory effect of an anti-cancer drug in a cancer cell, e.g., a KSP inhibitor in cancer cells.

The method of the invention can be used for evaluating a plurality of different agents. For example, sensitivity to a plurality of different DNA damaging agents described in Section 5.4.2., infra, may be evaluated by the method of the invention. In a preferred embodiment, sensitivity of each knockdown cell in the knockdown library to each of the plurality of different agents is evaluated to generate a two-dimensional responsiveness matrix comprising measurement of effect of each agent on each knockdown cell. A cut at the gene axis at a particular gene index gives a profile of responses of the particular knockdown cell (in which the particular gene is knocked down) to different drugs. A cut at the drug axis at a particular drug gives a gene responsiveness profile to the drug, i.e., a profile comprising measurements of effect of the drug on different knockdown cells in the knockdown library. Tables IIA-IIC are examples of gene responsiveness profiles to cisplatin (Table IIA), camptothecin (Table IIB), and doxorubicin (Table IIC).

The method of the invention may be used for identifying interaction between different genes by using an agent that regulates, e.g., suppresses or enhances, the expression of a gene and/or an activity of a protein encoded by the gene. Examples of such agents include but are not limited to siRNA, antisense, ribozyme, antibody, and small organic or inorganic molecules that target the gene or its product. The gene targeted by such an agent is termed the primary target. Such an agent can be used in conjunction with a knockdown library to identify gene or genes which modulates the response of the cell to the agent. The primary target can be different from any of the plurality of genes represented in the knockdown library (secondary genes). The gene or genes identified as modulating the effect of the agent are therefore gene or genes that interact with the primary target.

In a preferred embodiment, the invention provides a method for indentifying interaction between different genes using a dual siRNA approach. In a preferred embodiment, dual RNAi screens is achieved through the use of stable in vivo delivery of an shRNA disrupting the primary target gene and supertransfection of an siRNA targeting a secondary target gene. This approach provides matched (isogenic) cell line pairs (plus or minus the shRNA) and does not result in competition between the shRNA and siRNA. In the method, short hairpin RNAs (shRNAs) are expressed from recombinant vectors introduced either transiently or stably integrated into the genome (see, e.g., Paddison et al., 2002, Genes Dev 16:948-958; Sui et al., 2002, Proc Natl Acad Sci USA 99:5515-5520; Yu et al., 2002, Proc Natl Acad Sci USA 99:6047-6052; Miyagishi et al., 2002, Nat Biotechnol 20:497-500; Paul et al., 2002, Nat Biotechnol 20:505-508; Kwak et al., 2003, J Pharmacol Sci 93:214-217; Brummelkamp et al., 2002, Science 296:550-553; Boden et al., 2003, Nucleic Acids Res 31:5033-5038; Kawasaki et al., 2003, Nucleic Acids Res 31:700-707). The siRNA that disrupts the primary gene can be expressed (via an shRNA) by any suitable vector which encodes the shRNA. The vector can also encode a marker which can be used for selecting clones in which the vector or a sufficient portion thereof is integrated in the host genome such that the shRNA is expressed. Any standard method known in the art can be used to deliver the vector into the cells. In one embodiment, cells expressing the shRNA are generated by transfecting suitable cells with a plasmid containing the vector. Cells can then be selected by the appropriate marker. Clones are then picked, and tested for knockdown. In a preferred embodiment, the expression of the shRNA is under the control of an inducible promoter such that the silencing of its target gene can be turned on when desired. Inducible expression of an siRNA is particularly useful for targeting essential genes.

In one embodiment, the expression of the shRNA is under the control of a regulated promoter that allows tuning of the silencing level of the target gene. This allows screening against cells in which the target gene is partially knocked out. As used herein, a “regulated promoter” refers to a promoter that can be activated when an appropriate inducing agent is present. An “inducing agent” can be any molecule that can be used to activate transcription by activating the regulated promoter. An inducing agent can be, but is not limited to, a peptide or polypeptide, a hormone, or an organic small molecule. An analogue of an inducing agent, i.e., a molecule that activates the regulated promoter as the inducing agent does, can also be used. The level of activity of the regulated promoter induced by different analogues may be different, thus allowing more flexibility in tuning the activity level of the regulated promoter. The regulated promoter in the vector can be any mammalian transcription regulation system known in the art (see, e.g., Gossen et al, 1995, Science 268:1766-1769; Lucas et al, 1992, Annu. Rev. Biochem. 61:1131; Li et al., 1996, Cell 85:319-329; Saez et al., 2000, Proc. Natl. Acad. Sci. USA 97:14512-14517; and Pollock et al., 2000, Proc. Natl. Acad. Sci. USA 97:13221-13226). In preferred embodiments, the regulated promoter is regulated in a dosage and/or analogue dependent manner. In one embodiment, the level of activity of the regulated promoter is tuned to a desired level by a method comprising adjusting the concentration of the inducing agent to which the regulated promoter is responsive. The desired level of activity of the regulated promoter, as obtained by applying a particular concentration of the inducing agent, can be determined based on the desired silencing level of the target gene.

In one embodiment, a tetracycline regulated gene expression system is used (see, e.g., Gossen et al, 1995, Science 268:1766-1769; U.S. Pat. No. 6,004,941). A tet regulated system utilizes components of the tet repressor/operator/inducer system of prokaryotes to regulate gene expression in eukaryotic cells. Thus, the invention provides methods for using the tet regulatory system for regulating the expression of an shRNA linked to one or more tet operator sequences. The methods involve introducing into a cell a vector encoding a fusion protein that activates transcription. The fusion protein comprises a first polypeptide that binds to a tet operator sequence in the presence of tetracycline or a tetracycline analogue operatively linked to a second polypeptide that activates transcription in cells. By modulating the concentration of a tetracycline, or a tetracycline analogue, expression of the tet operator-linked shRNA is regulated.

In other embodiments, an ecdyson regulated gene expression system (see, e.g., Saez et al., 2000, Proc. Natl. Acad. Sci. USA 97:14512-14517), or an MMTV glucocorticoid response element regulated gene expression system (see, e.g., Lucas et al, 1992, Annu. Rev. Biochem. 61:1131) may be used to regulate the expression of the shRNA.

In one embodiment, a pRETRO-SUPER (pRS) vector which encodes a puromycin-resistance marker and drives shRNA expression from an H1 (RNA Pol III) promoter is used. The pRS-shRNA plasmid can be generated by any standard method known in the art. In one embodiment, the pRS-shRNA is deconvoluted from a library plasmid pool for a chosen gene by transforming bacteria with the pool and looking for clones containing only the plasmid of interest. Preferably, a 19mer siRNA sequence is used along with suitable forward and reverse primers for sequence specific PCR. Plasmids are identified by sequence specific PCR, and confirmed by sequencing. Cells expressing the shRNA are generated by transfecting suitable cells with the pRS-shRNA plasmid. Cells are selected by the appropriate marker, e.g., puromycin, and maintained until colonies are evident. Clones are then picked, and tested for knockdown.

In another embodiment, an shRNA is expressed by a plasmid, e.g., a pRS-shRNA. The knockdown by the pRS-shRNA plasmid, can be achieved by transfecting cells using Lipofectamine 2000 (Invitrogen).

In a preferred embodiment, matched cell lines (+/− primary target gene) are generated by selecting stable clones containing either empty pRS vector or pRS-shRNA.

Silencing of the secondary target gene are then carried out using cells of a generated shRNA primary target clone. Silencing of the secondary target gene can be achieved using any known method of RNA interference (see, e.g., Section 5.2.). For example, secondary target gene can be silenced by transfection with siRNA and/or plasmid encoding an shRNA. In one embodiment, cells of a generated shRNA primary target clone are supertransfected with one or more siRNAs targeting a secondary target gene. In one embodiment, the one or more siRNAs targeting the secondary gene are transfected into the cells directly. In another embodiment, the one or more siRNAs targeting the secondary gene are transfected into the cells via shRNAs using one or more suitable plasmids. RNA can be harvested 24 hours post transfection and knockdown assessed by TaqMan analysis. In a preferred embodiment, an siRNA pool containing at least k (k=2, 3, 4, 5, 6 or 10) different siRNAs targeting the secondary target gene at different sequence regions is used to supertransfect the cells. In another preferred embodiment, an siRNA pool containing at least k (k=2, 3, 4, 5, 6 or 10) different siRNAs targeting two or more different secondary target genes is used to supertransfect the cells.

In a preferred embodiment, the total siRNA concentration of the pool is about the same as the concentration of a single siRNA when used individually, e.g., 100 nM. Preferably, the total concentration of the pool of siRNAs is an optimal concentration for silencing the intended target gene. An optimal concentration is a concentration further increase of which does not increase the level of silencing substantially. In one embodiment, the optimal concentration is a concentration further increase of which does not increase the level of silencing by more than 5%, 10% or 20%. In a preferred embodiment, the composition of the pool, including the number of different siRNAs in the pool and the concentration of each different siRNA, is chosen such that the pool of siRNAs causes less than 30%, 20%, 10% or 5%, 1%, 0.1% or 0.01% of silencing of any off-target genes. In another preferred embodiment, the concentration of each different siRNA in the pool of different siRNAs is about the same. In still another preferred embodiment, the respective concentrations of different siRNAs in the pool are different from each other by less than 5%, 10%, 20% or 50%. In still another preferred embodiment, at least one siRNA in the pool of different siRNAs constitutes more than 90%, 80%, 70%, 50%, or 20% of the total siRNA concentration in the pool. In still another preferred embodiment, none of the siRNAs in the pool of different siRNAs constitutes more than 90%, 80%, 70%, 50%, or 20% of the total siRNA concentration in the pool. In other embodiments, each siRNA in the pool has an concentration that is lower than the optimal concentration when used individually. In a preferred embodiment, each different siRNA in the pool has an concentration that is lower than the concentration of the siRNA that is effective to achieve at least 30%, 50%, 75%, 80%, 85%, 90% or 95% silencing when used in the absence of other siRNAs or in the absence of other siRNAs designed to silence the gene. In another preferred embodiment, each different siRNA in the pool has a concentration that causes less than 30%, 20%, 10% or 5% of silencing of the gene when used in the absence of other siRNAs or in the absence of other siRNAs designed to silence the gene. In a preferred embodiment, each siRNA has a concentration that causes less than 30%, 20%, 10% or 5% of silencing of the target gene when used alone, while the plurality of siRNAs causes at least 80% or 90% of silencing of the target gene.

In one embodiment, the invention provides a method for identifying one or more genes which exhibit synthetic lethal interaction with a primary target gene. In the method, an agent that is an inhibitor of the primary target gene in the cell type is used to screen against a knockdown library. The gene or genes identified as enhancing the effect of the agent are therefore gene or genes that have synthetic lethal interaction with the primary target. In a preferred embodiment, the agent is an siRNA targeting and silencing the primary target.

The method for determining the effect of an agent on cells depends on the particular effect to be evaluated. For example, if the agent is an anti-cancer drug, and the effect to be evaluated is the growth inhibitory effect of the drug, an MTT assay or an alamarBlue assay may be used (see, e.g., Section 5.2). One skilled person in the art will be able to choose a method known in the art based on the particular effect to be evaluated.

In another embodiment, the invention provides a method of determining the effect of an agent on the growth of cells having the primary target gene and the secondary target gene silenced. In a preferred embodiment, matched cell lines (+/− primary target gene) are generated as described above. Both cell lines are then supertransfected with either a control siRNA (e.g., luciferase) or one or more siRNAs targeting a secondary target gene. The cell cycle profiles are examined with or without exposure to the agent. Cell cycle analysis can be carried out using standard method known in the art (see, Section 5.2., infra). In one embodiment, the supernatant from each well is combined with the cells that have been harvested by trypsinization. The mixture is then centrifuged at a suitable speed. The cells are then fixed with ice cold 70% ethanol for a suitable period of time, e.g., ˜30 minutes. Fixed cells can be washed once with PBS and resuspended, e.g., in 0.5 ml of PBS containing Propidium Iodide (10 microgram/ml) and RNase A(1 mg/ml), and incubated at a suitable temperature, e.g., 37° C., for a suitable period of time, e.g., 30 min. Flow cytometric analysis is carried out using a flow cytometer. In one embodiment, the Sub-G1 cell population is used to measure cell death. An increase of sub-G1 cell population in cells having the primary target gene and the secondary target gene silenced indicates synthetic lethality between the primary and secondary target genes in the presence of the agent.

In a specific embodiment, the invention provides a method for identifying gene or genes whose knockdown enhances the growth inhibitory effect of a KSP inhibitor on tumor cells. In one embodiment, the method was used to identify genes whose knockdown inhibits tumor cell growth in the presence of suboptimal concentrations of a KSPi, i.e., concentrations lower than EC10. In one embodiment, an siRNA knockdown library contained 3 siRNAs targeting each of the following 11 genes: CDC20, ROCK2, TTK, FZR1, BUB1, BUB3, BUB1B, MAD1L1, MAD2L1, DNCH1 and STK6 are generated and used (see Table I). Each of these siRNAs were transfected into HeLa cells in the presence or absence of an <EC10 concentration (25 nM) of a KSPi, (1S)-1-{[(2S)-4-(2,5-difluorophenyl)-2-phenyl-2,5-dihydro-1H-pyrrol-1-yl]carbonyl}-2-methylpropylamine (see, PCT application PCT/US03/18482, filed Jun. 12, 2003) (EC50˜80 nM) and the response of the cell was determined. One siRNA to STK6 (STK6-1) showed significant inhibition of tumor cell growth in the presence of KSPi.

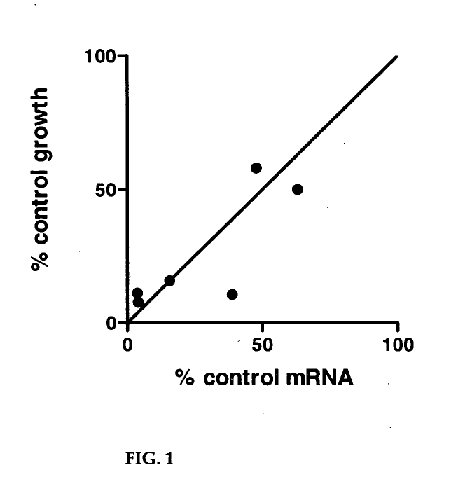

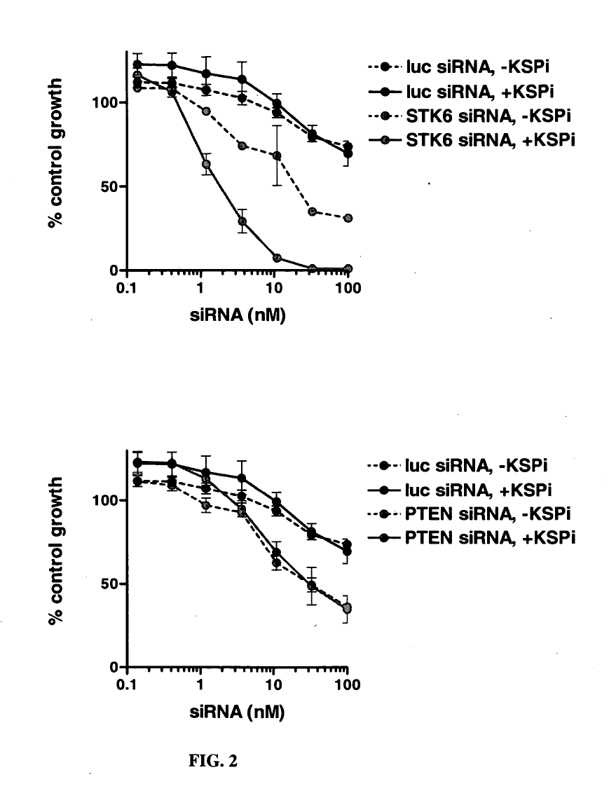

The growth inhibitory activity was further examined using three additional siRNAs to STK6 and the abilities of all six siRNAs to induce STK6 silencing and growth inhibition were evaluated. Amongst the different siRNAs, there was a good correlation between the level of STK6 silencing and growth inhibition (FIG. 1). This correlation suggested that growth inhibition was due to on target activity (i.e., STK6 disruption). STK6-1 was then titrated with control siRNAs targeting luciferase (negative control) in the presence or absence of (1S)-1-{[(2S)-4-(2,5-difluorophenyl)-2-phenyl-2,5-dihydro-1H-pyrrol-1-yl]carbonyl}-2-methylpropylamine as described in PCT application PCT/US03/18482, filed Jun. 12, 2003. (FIG. 2). The addition of KSPi shifted the STK6-1 dose response curve 5-10-fold to the left. This concentration of KSPi did not augment effects on cell growth caused by a luciferase siRNA. In contrast, the dose response curve to a siRNA targeting PTEN with similar effects on cell growth as STK6-1 was not shifted by KSPi. Other siRNAs targeting STK6 also enhanced the effect of KSPi on cell growth. Thus, disruption of STK6 enhances the effect of KSPi on cell growth. Further support for this was obtained by studies using combinations of siRNAs to STK6 and KSP (Table I), which showed greater growth inhibitory activity than either siRNA alone. Because the concentrations of KSPi used in these experiments did not affect cell growth on its own, the effects of KSPi on STK6 siRNA activity appeared synergistic rather than additive.

In another specific embodiment, the invention provides a method for determining synthetic lethality between p53 and CHEK1. Stable clones having p53 gene silenced was generated. The pRS-TP53 1026 shRNA plasmid was deconvoluted from a library plasmid pool for TP53 by transforming bacteria with the pool and looking for clones containing only the plasmid of interest. The sequences used are as follows: pRS-p53 1026 19mer sequence: GACTCCAGTGGTAATCTAC (SEQ ID NO:43); primers for sequence specific PCR: Forward: GTAGATTACCACTGGAGTC (SEQ ID NO:44), Reverse: CCCTTGAACCTCCTCGTTCGACC (SEQ ID NO:45). Plasmids were identified by sequence specific PCR, and confirmed by sequencing. Stable p53-clones were generated by transfecting HCT116 cells using FuGENE 6 (Roche) with the pRS-TP53 1026 plasmid. Cells were split into 10 cm dishes plus 1 ug/ml puromycin 48 hours later, and maintained until colonies were evident (5-7 days). Clones were picked into a 96 well plate, maintained in 1 ug/ml puromycin, and tested for knockdown by TaqMan using the TP53 and hGUS Pre-Developed Assay Reagent (Applied Biosystems). To measure transient knockdown by the pRS-TP53 1026 plasmid, HCT116 cells were transfected using Lipofectamine 2000 (Invitrogen), and RNA harvested 24 hours later. TP53 transcript levels were assessed by TaqMan.

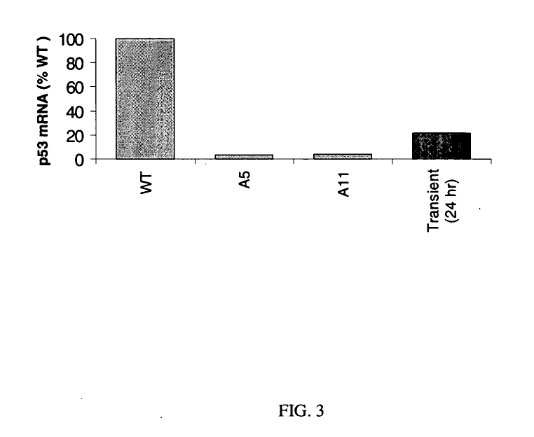

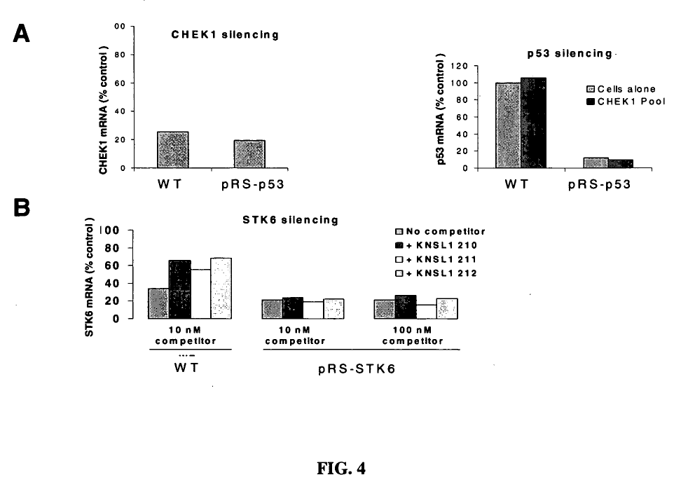

Analysis of multiple puromycin-resistant TP53 shRNA clones (pRS-p53) derived from the colon tumor line HCT116 showed varying levels of target silencing (50% to 96% as determined by TaqMan). FIG. 3 shows the level of TP53 expression in clones A5 and A11, which exhibited the highest levels of silencing. TP53 silencing achieved in these clones exceeded that observed 24 hr after delivery of pRS-p53 into HCT116 cells by transient transfection (FIG. 3). It is possible that transfection efficiency limits the effectiveness of TP53 shRNA in transient assays. Alternatively, cells having greater levels of TP53 silencing gain a growth advantage during clonal growth. With an shRNA that targets STK6 (pRS-STK6: pRS-STK6 2178 19mer sequence: CATTGGAGTCATAGCATGT (SEQ ID NO:46)), a range of silencing in stable clones was also observed. These clones, however, did not achieve as high a degree of silencing observed in the TP53 lines, nor was silencing greater than that achieved in transient assays. This may indicate selection against high level of STK6 silencing because STK6 is an essential gene for tumor cell growth in culture.

To test whether TP53 silencing in HCT116 clone A11 was subject to competition with siRNAs, cells of this clone were supertransfected with a pool of CHEK1-specific siRNAs. CHK1 pool contains the following three siRNAs: CUGAAGAAGCAGUCGCAGUTT (SEQ ID NO:99); AUCGAUUCUGCUCCUCUAGTT (SEQ ID NO:98); and UGCCUGAAAGAGACUUGUGTT (SEQ ID NO:100). This siRNA pool had been found to competitively reduce silencing activity of a TP53 targeted siRNA. siRNAs were transfected using Oligofectamine (Invitrogen) at 10 nM or 100 nM where indicated. For the CHK1 pool, three siRNAs were transfected simultaneously at 33.3 nM each for a total delivery of 100 nM. RNA was harvested 24 hours post transfection and knockdown was assessed by TaqMan analysis using the CHK1 or TP53 and hGUS Pre-Developed Assay Reagent (Applied Biosystems). As shown in FIG. 4A, the shRNA and the siRNA pool did not competitively inhibit silencing of each other's targets. Inhibition by known competitive siRNAs of either a transiently transfected siRNA or a stably expressed shRNA of the same sequence was then assayed. As shown in FIG. 4B, three individual siRNAs targeting KNSL1 (KNSLI 210: GACCUGUGCCUUUUAGAGATT (SEQ ID NO:47); KNSLI 211: GACUUCAUUGACAGUGGCCTT (SEQ ID NO:48); KNSLI 212: AAAGGACAACUGCAGCUACTT (SEQ ID NO:49)) competitively inhibited the silencing achieved by co-transfected siRNA targeting STK6 (left bars). In contrast, silencing by the homologous STK6 shRNA in stably transfected lines was unaffected by supertransfection of the KNSL1 siRNAs, even when the competitor siRNAs were added at ten fold higher concentrations (middle and right bars). These experiments suggested that there was little competition between stably expressed shRNAs and transiently transfected siRNAs. This is in contrast to the observation that two different siRNAs targeting distinct mRNAs compete with each other when transfected together, effectively decreasing the efficacy of one or both of the siRNAs used. pRS and pRS-p53 HCT116 cells were transiently transfected with siRNA pools for ˜800 genes (see Example 3, infra) and measured effects on cellular growth by Alamar Blue assay. Nearly identical responses to the ˜800 siRNA pools in pRS cells and in pRS-p53 cells, with no suggestion of competitive inhibition of silencing were observed.

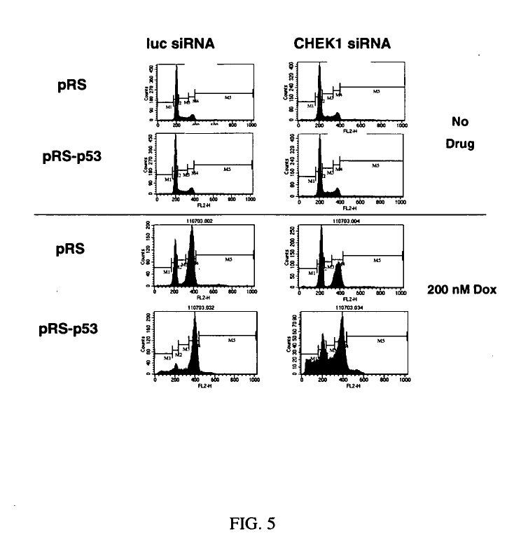

Next, supertransfection of the CHEK1 siRNA pool into cells stably expressing TP53 shRNAs was evaluated to determine if it could be used to investigate genetic interactions (SL) between these molecules. Matched cell lines +/−TP53 expression were generated by selecting stable clones of A549 lung cancer cell lines containing either empty pRS vector or pRS-p53. The latter cells showed >90% silencing of TP53 mRNA. Both cell lines were then supertransfected with either control luciferase siRNA (luc, 100 nM) or the CHEK1 siRNA pool (100 nM total; 33 nM each of 3 siRNAs) and their cell cycle profiles examined with or without exposure to the DNA damaging agent, doxorubicin (Dox, FIG. 5). Cell cycle profiles of pRS-p53 cells were not appreciably different from those of pRS cells in the absence of Dox. Transient transfection of CHEK1 siRNAs also did not affect cell cycle profiles in the absence of Dox. In the presence of Dox, however, pRS-transfected cells exhibited G1 and G2/M arrest as is expected of cells expressing functional TP53. Supertransfection of CHEK1 siRNAs resulted in an override of the G2 checkpoint and an increase in the number of cells blocked at G1. Because the cells retained TP53 function, they stopped in G1 and did not proceed back into S phase.

In contrast, pRS-p53 cells lost the ability to arrest at G1 and arrested primarily at G2 in response Dox treatment, consistent with the role of TP53 in the G1 checkpoint. The cell cycle profile of pRS-p53 cells was unchanged by supertransfection of luc siRNA (FIG. 5). The failure of luc siRNA to cause even partial restoration of the TP53 response (and a corresponding increase in the G1 peak) suggests that there was little competitive inhibition of TP53 silencing and phenotype by this siRNA. Therefore, competitive inhibition of TP53 silencing by the CHEK1 siRNA pool was not expected to exist. Indeed, in response to Dox treatment, pRS-p53 cells transiently transfected with CHEK1 showed profound alterations in their cell cycle profile with large increases in the fraction of cells in S and with sub-G1 (dead cells) amounts of DNA. Similar findings were also observed in pRS and pRS-p53 stably transfected HCT116 cells. Thus, simultaneous disruption of the G1 checkpoint mediated by TP53 and the G2 checkpoint mediated by CHEK1 is lethal to TP53− but not TP53+ tumor cells.

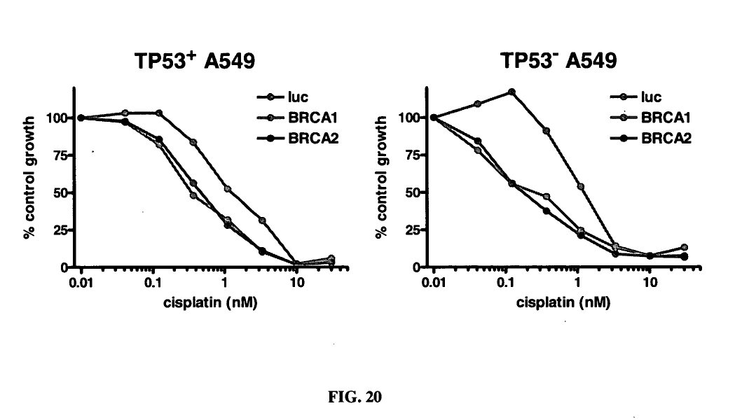

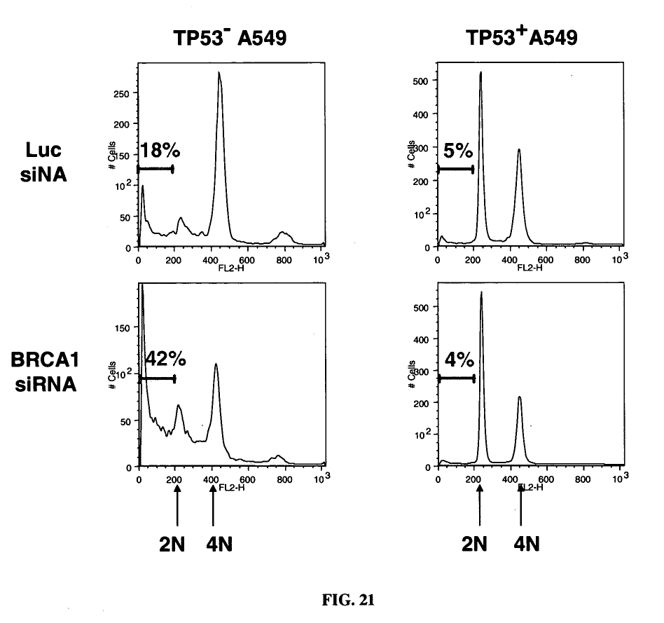

In another embodiment, the invention provides a method for determining synthetic lethality between p53 and a member of the BRCC complex, e.g., BRCA1, BRCA2, BARD1 and RAD51. In this embodiment, a matched pair of TP53 positive and negative cells generated by stable expression of short hairpin RNAs (shRNAs) targeting TP53 was used. TP53-positive or negative cells were supertransfected with siRNA pools to BRCA1 or BRCA2, treated with cisplatin and analyzed for cell growth using Alamar Blue (FIG. 20). TP53-negative cells were ˜10-fold more sensitive to cisplatin when transfected with BRCA1 or BRCA2 siRNAs (IC50˜0.1 nM) than with control siRNA (luciferase, IC50-˜1 nM). The sensitization to cisplatin following BRCA1 or BRCA2 disruption was even more pronounced at lower cisplatin concentrations. TP53-positive cells were less sensitized to cisplatin following BRCA1 or BRCA2 disruption (IC50 ˜0.4 nM). Sensitization to cisplatin following BRCA1 or BRCA2 disruption was similar in magnitude in this assay to the sensitization seen following disruption of CHEK1 (data not shown). Sensitization to DNA damaging agents following BRCA1 and BRCA2 disruption can also be investigated using cell cycle analysis. TP53-positive and negative cells were supertransfected with siRNA pools to BRCA1 or BRCA2, treated with one of several DNA damaging agents (cisplatin, camptothecin, doxorubicin and bleomycin) and analyzed for cell cycle distribution by flow cytometry. In all cases, TP53-negative cells were more sensitive to DNA damage following BRCA1 or BRCA2 disruption than in luciferase-transfected cells (data not shown). The response of these cells to bleomycin following BRCA1 disruption is shown in FIG. 21. BRCA1 disruption resulted in more sub-G1 cells (dead cells) following bleomycin treatment of TP53-negative than TP53-positive cells. The results show that cells lacking TP53 are more sensitive to DNA damage following BRCA1 disruption.

The cell lines used can be HeLa cells, TP53-positive A549 cells or TP53-negative A549 cells. In one embodiment, matched pair of TP53 positive and negative cells were generated by stable transfection of short hairpin RNAs (shRNAs) targeting TP53 (monthly highlt highlight, November 2003). The cells were transfected using pools of siRNAs (pool of 3 siRNA per gene) at 100 nM (each siRNA at 33 nM), or with single siRNA at 100 nM. The following siRNAs were used: Luc control, BRCA1, BRCA2 and BARD1 pool. These transfected cells were then treated with varying concentrations of DNA damaging agents. The concentration for each agent used in the cell cycle analysis is as follows: for HeLa cells, Doxorubicin (10 nM), Camptothecin (6 nM), Cisplatin (400 ng/ml), Mitomycin C (40 nM), Bleomycin (100 ng/ml); for the other cell lines, Doxorubicin (200 nM), Camptothecin (200 nM), Cisplatin (2 ug/ml), Mitomycin C (400 nM), Bleomycin (5 ug/ml).

In one embodiment, siRNA transfection was carried out as follows: one day prior to transfection, 2000 (or 100) microliters of a chosen cell line, e.g., cervical cancer HeLa cells (ATCC, Cat. No. CCL-2), grown in DMEM/10% fetal bovine serum (Invitrogen, Carlsbad, Calif.) to approximately 90% confluency were seeded in a 6-well (or 96-well) tissue culture plate at 45,000 (or 2000) cells/well. For each transfection 70 microliters of OptiMEM (Invitrogen) was mixed with 5 microliter of siRNA (Dharmacon, Lafayette, Colo.) from a 20 micromolar stock. For each transfection, a ratio of 20 microliter of OptiMEM was mixed with 5 microliter of Oligofectamine reagent (Invitrogen) and incubated 5 minutes at room temperature. Then 25-microliter OptiMEM/Oligofectamine mixture was mixed with the 75-microliter of OptiMEM/siRNA mixture, and incubated 15-20 minutes at room temperature. 100 (or 10) microliter of the transfection mixture was aliquoted into each well of the 6-well (or 96-well) plate and incubated for 4 hours at 37° C. and 5% CO₂.

After 4 hours, 100 microliter/well of DMEM/10% fetal bovine serum with or without DNA damage agents was added to each well to reach the final concentration of each agents as described above. The plates were incubated at 37° C. and 5% CO₂ for another 68 hours. Samples from the 6-well plates were analyzed for cell cycle profiles and samples from 96-well plates were analyzed for cell growth with Alamar Blue assay.

For cell cycle analysis, the supernatant from each well was combined with the cells that were harvested by trypsinization. The mixture was then centrifuged at 1200 rpm for 5 minutes. The cells were then fixed with ice cold 70% ethanol for ˜30 minutes. Fixed cells were washed once with PBS and resuspended in 0.5 ml of PBS containing Propidium Iodide (10 microgram/ml) and RNase A (1 mg/ml), and incubated at 37° C. for 30 min. Flow cytometric analysis was carried out using a FACSCalibur flow cytometer (Becton Dickinson) and the data was analyzed using FlowJo software (Tree Star, Inc). The Sub-G1 cell population was used to measure cell death. If the summation of the Sub-G1 population from the (siRNA+DMSO) sample and (Luc+drug) sample is larger than the Sub-G1 population of (siRNA+drug) sample, we define that as sensitization of siRNA silencing to DNA damage.

For Alamar Blue assay, the media from the 96-well plates was removed, and 100 uL/well complete media containing 10% (vol/vol) alamarBlue reagent (BioSource International, Inc) and 1/100^{th} volume 1M Hepes buffer tissue culture reagent was added. The plates were then incubated 1-4 hours at 37° C. and fluorescence was measured by exciting at 544 nm and detecting emission at 590 nm with SPECTRAMax Gemini-Xs Spectrofluorometer (Molceular Devices). The fluorescence signal was corrected for background (no cells). Cell response (survival) in the presence of DNA damaging agents was measured as a percentage of control cell growth in the absence of DNA damaging agents.

### 5.2. Methods and Compositions for RNA Interference and Cell Assays

Any standard method for gene silencing can be used in the present invention (see, e.g., Guo et al., 1995, Cell 81:611-620; Fire et al., 1998, Nature 391:806-811; Grant, 1999, Cell 96:303-306; Tabara et al., 1999, Cell 99:123-132; Zamore et al., 2000, Cell 101:25-33; Bass, 2000, Cell 101:235-238; Petcherski et al., 2000, Nature 405:364-368; Elbashir et al., Nature 411:494-498; Paddison et al., Proc. Natl. Acad. Sci. USA 99:1443-1448). The siRNAs targeting a gene can be designed according to methods known in the art (see, e.g., U.S. Provisional Patent Application No. 60/572,314 by Jackson et al., filed on May 17, 2004, and Elbashir et al., 2002, Methods 26:199-213, each of which is incorporated herein by reference in its entirety).

SiRNAs having only partial sequence homology to a target gene can also be used (see, e.g., International application No. PCT/U.S. 2004/015439 by Jackson et al., filed on May 17, 2004, which is incorporated herein by reference in its entirety). In one embodiment, an siRNA that comprises a sense strand contiguous nucleotide sequence of 11-18 nucleotides that is identical to a sequence of a transcript of a gene but the siRNA does not have full length homology to any sequences in the transcript is used to silence the gene. Preferably, the contiguous nucleotide sequence is in the central region of the siRNA molecules. A contiguous nucleotide sequence in the central region of an siRNA can be any continuous stretch of nucleotide sequence in the siRNA which does not begin at the 3′ end. For example, a contiguous nucleotide sequence of 11 nucleotides can be the nucleotide sequence 2-12, 3-13, 4-14, 5-15, 6-16, 7-17, 8-18, or 9-19. In preferred embodiments, the contiguous nucleotide sequence is 11-16, 11-15, 14-15, 11, 12, or 13 nucleotides in length.

In another embodiment, an siRNA that comprises a 3′ sense strand contiguous nucleotide sequence of 9-18 nucleotides which is identical to a sequence of a transcript of a gene but which siRNA does not have full length sequence identity to any contiguous sequences in the transcript is used to silence the gene. In this application, a 3′ 9-18 nucleotide sequence is a continuous stretch of nucleotides that begins at the first paired base, i.e., it does not comprise the two base 3′ overhang. Thus, when it is stated that a particular nucleotide sequence is at the 3′ end of the siRNA, the 2 base overhang is not considered. In preferred embodiments, the contiguous nucleotide sequence is 9-16, 9-15, 9-12, 11, 10, or 9 nucleotides in length.

Any method known in the art can be used for carrying out RNA interference. In one embodiment, gene silencing is induced by presenting the cell with the siRNA, mimicking the product of Dicer cleavage (see, e.g., Elbashir et al., 2001, Nature 411, 494-498; Elbashir et al., 2001, Genes Dev. 15, 188-200, all of which are incorporated by reference herein in their entirety). Synthetic siRNA duplexes maintain the ability to associate with RISC and direct silencing of mRNA transcripts. siRNAs can be chemically synthesized, or derived from cleavage of double-stranded RNA by recombinant Dicer. Cells can be transfected with the siRNA using standard method known in the art.

In one embodiment, siRNA transfection is carried out as follows: one day prior to transfection, 100 microliters of chosen cells, e.g., cervical cancer HeLa cells (ATCC, Cat. No. CCL-2), grown in DMEM/10% fetal bovine serum (Invitrogen, Carlsbad, Calif.) to approximately 90% confluency are seeded in a 96-well tissue culture plate (Corning, Corning, N.Y.) at 1500 cells/well. For each transfection 85 microliters of OptiMEM (Invitrogen) is mixed with 5 microliter of serially diluted siRNA (Dharma on, Denver) from a 20 micro molar stock. For each transfection 5 microliter OptiMEM is mixed with 5 microliter Oligofectamine reagent (Invitrogen) and incubated 5 minutes at room temperature. The 10 microliter OptiMEM/Oligofectamine mixture is dispensed into each tube with the OptiMEM/siRNA mixture, mixed and incubated 15-20 minutes at room temperature. 10 microliter of the transfection mixture is aliquoted into each well of the 96-well plate and incubated for 4 hours at 37° C. and 5% CO₂.

Another method for gene silencing is to introduce an shRNA, for short hairpin RNA (see, e.g., Paddison et al., 2002, Genes Dev. 16, 948-958; Brummelkamp et al., 2002, Science 296, 550-553; Sui, G. et al. 2002, Proc. Natl. Acad. Sci. USA 99, 5515-5520, all of which are incorporated by reference herein in their entirety), which can be processed in the cells into siRNA. In this method, a desired siRNA sequence is expressed from a plasmid (or virus) as an inverted repeat with an intervening loop sequence to form a hairpin structure. The resulting RNA transcript containing the hairpin is subsequently processed by Dicer to produce siRNAs for silencing. Plasmid-based shRNAs can be expressed stably in cells, allowing long-term gene silencing in cells both in vitro and in vivo, e.g., in animals (see, McCaffrey et al. 2002, Nature 418, 38-39; Xia et al., 2002, Nat. Biotech. 20, 1006-1010; Lewis et al., 2002, Nat. Genetics 32, 107-108; Rubinson et al., 2003, Nat. Genetics 33, 401-406; Tiscomia et al., 2003, Proc. Natl. Acad. Sci. USA 100, 1844-1848, all of which are incorporated by reference herein in their entirety). Thus, in one embodiment, a plasmid-based shRNA is used.

In a preferred embodiment, shRNAs are expressed from recombinant vectors introduced either transiently or stably integrated into the genome (see, e.g., Paddison et al., 2002, Genes Dev 16:948-958; Sui et al., 2002, Proc Natl Acad Sci USA 99:5515-5520; Yu et al., 2002, Proc Natl Acad Sci USA 99:6047-6052; Miyagishi et al., 2002, Nat Biotechnol 20:497-500; Paul et al., 2002, Nat Biotechnol 20:505-508; Kwak et al., 2003, J Pharmacol Sci 93:214-217; Brummelkamp et al., 2002, Science 296:550-553; Boden et al., 2003, Nucleic Acids Res 31:5033-5038; Kawasaki et al., 2003, Nucleic Acids Res 31:700-707). The siRNA that disrupts the target gene can be expressed (via an shRNA) by any suitable vector which encodes the shRNA. The vector can also encode a marker which can be used for selecting clones in which the vector or a sufficient portion thereof is integrated in the host genome such that the shRNA is expressed. Any standard method known in the art can be used to deliver the vector into the cells. In one embodiment, cells expressing the shRNA are generated by transfecting suitable cells with a plasmid containing the vector. Cells can then be selected by the appropriate marker. Clones are then picked, and tested for knockdown. In a preferred embodiment, the expression of the shRNA is under the control of an inducible promoter such that the silencing of its target gene can be turned on when desired. Inducible expression of an siRNA is particularly useful for targeting essential genes.

In one embodiment, the expression of the shRNA is under the control of a regulated promoter that allows tuning of the silencing level of the target gene. This allows screening against cells in which the target gene is partially knocked out. As used herein, a “regulated promoter” refers to a promoter that can be activated when an appropriate inducing agent is present. An “inducing agent” can be any molecule that can be used to activate transcription by activating the regulated promoter. An inducing agent can be, but is not limited to, a peptide or polypeptide, a hormone, or an organic small molecule. An analogue of an inducing agent, i.e., a molecule that activates the regulated promoter as the inducing agent does, can also be used. The level of activity of the regulated promoter induced by different analogues may be different, thus allowing more flexibility in tuning the activity level of the regulated promoter. The regulated promoter in the vector can be any mammalian transcription regulation system known in the art (see, e.g., Gossen et al, 1995, Science 268:1766-1769; Lucas et al, 1992, Annu. Rev. Biochem. 61:1131; L1 et al., 1996, Cell 85:319-329; Saez et al., 2000, Proc. Natl. Acad. Sci. USA 97:14512-14517; and Pollock et al., 2000, Proc. Natl. Acad. Sci. USA 97:13221-13226). In preferred embodiments, the regulated promoter is regulated in a dosage and/or analogue dependent manner. In one embodiment, the level of activity of the regulated promoter is tuned to a desired level by a method comprising adjusting the concentration of the inducing agent to which the regulated promoter is responsive. The desired level of activity of the regulated promoter, as obtained by applying a particular concentration of the inducing agent, can be determined based on the desired silencing level of the target gene.

In one embodiment, a tetracycline regulated gene expression system is used (see, e.g., Gossen et al, 1995, Science 268:1766-1769; U.S. Pat. No. 6,004,941). A tet regulated system utilizes components of the tet repressor/operator/inducer system of prokaryotes to regulate gene expression in eukaryotic cells. Thus, the invention provides methods for using the tet regulatory system for regulating the expression of an shRNA linked to one or more tet operator sequences. The methods involve introducing into a cell a vector encoding a fusion protein that activates transcription. The fusion protein comprises a first polypeptide that binds to a tet operator sequence in the presence of tetracycline or a tetracycline analogue operatively linked to a second polypeptide that activates transcription in cells. By modulating the concentration of a tetracycline, or a tetracycline analogue, expression of the tet operator-linked shRNA is regulated.

In other embodiments, an ecdyson regulated gene expression system (see, e.g., Saez et al., 2000, Proc. Natl. Acad. Sci. USA 97:14512-14517), or an MMTV glucocorticoid response element regulated gene expression system (see, e.g., Lucas et al, 1992, Annu. Rev. Biochem. 61:1131) may be used to regulate the expression of the shRNA.

In one embodiment, the pRETRO-SUPER (pRS) vector which encodes a puromycin-resistance marker and drives shRNA expression from an H1 (RNA Pol III) promoter is used. The pRS-shRNA plasmid can be generated by any standard method known in the art. In one embodiment, the pRS-shRNA is deconvoluted from the library plasmid pool for a chosen gene by transforming bacteria with the pool and looking for clones containing only the plasmid of interest. Preferably, a 19mer siRNA sequence is used along with suitable forward and reverse primers for sequence specific PCR. Plasmids are identified by sequence specific PCR, and confirmed by sequencing. Cells expressing the shRNA are generated by transfecting suitable cells with the pRS-shRNA plasmid. Cells are selected by the appropriate marker, e.g., puromycin, and maintained until colonies are evident. Clones are then picked, and tested for knockdown. In another embodiment, an shRNA is expressed by a plasmid, e.g., a pRS-shRNA. The knockdown by the pRS-shRNA plasmid, can be achieved by transfecting cells using Lipofectamine 2000 (Invitrogen).

In yet another method, siRNAs can be delivered to an organ or tissue in an animal, such a human, in vivo (see, e.g., Song et al. 2003, Nat. Medicine 9, 347-351; Sorensen et al., 2003, J. Mol. Biol. 327, 761-766; Lewis et al., 2002, Nat. Genetics 32, 107-108, all of which are incorporated by reference herein in their entirety). In this method, a solution of siRNA is injected intravenously into the animal. The siRNA can then reach an organ or tissue of interest and effectively reduce the expression of the target gene in the organ or tissue of the animal.

Any suitable proliferation or growth inhibition assays known in the art can be used to assay cell growth. In a preferred embodiment, an MTT proliferation assay (see, e.g., van de Loosdrechet, et al., 1994, J. Immunol. Methods 174: 311-320; Ohno et al., 1991, J. Immunol. Methods 145:199-203; Ferrari et al., 1990, J. Immunol. Methods 131: 165-172; Alley et al., 1988, Cancer Res. 48: 589-601; Carmichael et al., 1987, Cancer Res. 47:936-942; Gerlier et al., 1986, J. Immunol. Methods 65:55-63; Mosmann, 1983, J. Immunological Methods 65:55-63) is used to assay the effect of one or more agents in inhibiting the growth of cells. The cells are treated with chosen concentrations of one or more candidate agents for a chosen period of time, e.g., for 4 to 72 hours. The cells are then incubated with a suitable amount of 3-(4,5-dimethylthiazol-2-yl)-2,5-diphenyltetrazolium bromide (MTT) for a chosen period of time, e.g., 1-8 hours, such that viable cells convert MTT into an intracellular deposit of insoluble formazan. After removing the excess MTT contained in the supernatant, a suitable MTT solvent, e.g., a DMSO solution, is added to dissolved the formazan. The concentration of MTT, which is proportional to the number of viable cells, is then measured by determining the optical density at e.g., 570 nm. A plurality of different concentrations of the candidate agent can be assayed to allow the determination of the concentrations of the candidate agent or agents which causes 50% inhibition.

In another preferred embodiment, an alamarBlue™ Assay for cell proliferation is used to screen for one or more candidate agents that can be used to inhibit the growth of cells (see, e.g., Page et al., 1993, Int. J. Oncol. 3:473-476). An alamarBlue™ assay measures cellular respiration and uses it as a measure of the number of living cells. The internal environment of proliferating cells is more reduced than that of non-proliferating cells. For example, the ratios of NADPH/NADP, FADH/FAD, FMNH/FMN, and NADH/NAF increase during proliferation. AlamarBlue can be reduced by these metabolic intermediates and, therefore, can be used to monitor cell proliferation. The cell number of a treated sample as measured by alamarBlue can be expressed in percent relative to that of an untreated control sample. alamarBlue reduction can be measured by either absorption or fluorescence spectroscopy. In one embodiment, the alamarBlue reduction is determined by absorbance and calculated as percent reduced using the equation:

$$\% Reduced = \frac{( {ɛ}_{ox} {λ}_{2} ) ( A {λ}_{1} ) - ( {ɛ}_{ox} {λ}_{1} ) ( A {λ}_{2} )}{( {ɛ}_{red} {λ}_{1} ) ( {A}^{′} {λ}_{2} ) - ( {ɛ}_{red} {λ}_{2} ) ( {A}^{′} {λ}_{1} )} \times 100 \qquad ( 1 )$$

where:
λ₁=570 nm
λ₂=600 nm
(ε_{red} λ₁)=155,677 (Molar extinction coefficient of reduced alamarBlue at 570 nm)
(ε_{red} λ₂)=14,652 (Molar extinction coefficient of reduced alamarBlue at 600 nm)
(εₒₓ λ₁)=80,586 (Molar extinction coefficient of oxidized alamarBlue at 570 nm)
(εₒₓ λ₂)=117,216 (Molar extinction coefficient of oxidized alamarBlue at 600 nm)
(A λ₁)=Absorbance of test wells at 570 nm
(A λ₂)=Absorbance of test wells at 600 nm
(A′λ₁)=Absorbance of negative control wells which contain medium plus alamar Blue but to which no cells have been added at 570 nm.
(A′λ₂)=Absorbance of negative control wells which contain medium plus alamar Blue but to which no cells have been added at 600 nm. Preferably, the % Reduced of wells containing no cell was subtracted from the % Reduced of wells containing samples to determine the % Reduced above background.

Cell cycle analysis can be carried out using standard method known in the art. In one embodiment, the supernatant from each well is combined with the cells that have been harvested by trypsinization. The mixture is then centrifuged at a suitable speed. The cells are then fixed with, e.g., ice cold 70% ethanol for a suitable period of time, e.g., ˜30 minutes. Fixed cells can be washed once with PBS and resuspended, e.g., in 0.5 ml of PBS containing Propidium Iodide (10 microgram/ml) and RNase A (1 mg/ml), and incubated at a suitable temperature, e.g., 37° C., for a suitable period of time, e.g., 30 min. Flow cytometric analysis is then carried out using a flow cytometer. In one embodiment, the Sub-G1 cell population is used as a measure of cell death. For example, the cells are said to have been sensitized to an agent if the Sub-G1 population from the sample treated with the agent is larger than the Sub-G1 population of sample not treated with the agent.

### 5.3. Uses of KSP Interacting Genes and their Products

The invention provides methods and compositions for utilizing a gene that interacts with KSP (“KSP interacting gene”), e.g., STK6 or TPX2 gene, its product and antibodies for identifying proteins or other molecules that interact with the KSP interacting gene or protein. In preferred embodiment, the invention provides STK6 or TPX2 gene as such KSP interacting gene. The invention also provides methods and compositions for utilizing the the KSP interacting gene, e.g., STK6 or TPX2 gene, product and antibodies for screening for agents that regulate expression of the KSP interacting gene or modulating interaction of the KSP interacting gene or protein with other proteins or molecules. The invention further provides methods and compositions for utilizing the KSP interacting gene, e.g., STK6 or TPX2 gene, product and antibodies for screening for agents that are useful in regulating resistance to the growth inhibitory effect of a KSP inhibitor (KSPi) and/or in enhancing the growth inhibitory effect of a KSP inhibitor in a cell or organism. The invention also provides methods and compositions for utilizing the KSP interacting gene, e.g., STK6 or TPX2 gene, product and antibodies for diagnosing resistance to the growth inhibitory effect of KSP inhibitors mediated by the KSP interacting gene, and for treatment of diseases in conjunction with a therapy using a KSP inhibitor.

### 5.3.1. Methods of Determining Proteins or Other Molecules that Interact with a KSP Interacting Gene or Its Product

Any method suitable for detecting protein-protein interactions may be employed for identifying interaction of a KSP interacting protein, e.g., STK6 or TPX2 protein, with another cellular protein. The interaction between a KSP interacting gene e.g., STK6 or TPX2 gene, and other cellular molecules, e.g., interaction between a KSP interacting gene and its regulators, may also be determined using methods known in the art.

Among the traditional methods which may be employed are co-immunoprecipitation, crosslinking and co-purification through gradients or chromatographic columns. Utilizing procedures such as these allows for the identification of cellular proteins which interact with a KSP interacting gene product. Once isolated, such a cellular protein can be identified and can, in turn, be used, in conjunction with standard techniques, to identify proteins it interacts with. For example, at least a portion of the amino acid sequence of the cellular protein which interacts with a KSP interacting gene product can be ascertained using techniques well known to those of skill in the art, such as via the Edman degradation technique (see, e.g., Creighton, 1983, “Proteins: Structures and Molecular Principles”, W.H. Freeman & Co., N.Y., pp. 34-49). The amino acid sequence obtained may be used as a guide for the generation of oligonucleotide mixtures that can be used to screen for gene sequences encoding such cellular proteins. Screening may be accomplished, for example, by standard hybridization or PCR techniques. Techniques for the generation of oligonucleotide mixtures and the screening are well-known. (See, e.g., Ausubel, supra., and PCR Protocols: A Guide to Methods and Applications, 1990, Innis, M. et al., eds. Academic Press, Inc., New York).

Additionally, methods may be employed which result in the simultaneous identification of genes which encode the cellular protein interacting with the KSP interacting protein. These methods include, for example, probing expression libraries with a labeled KSP interacting protein, using the KSP interacting protein in a manner similar to the well known technique of antibody probing of λgt11 libraries.

One method which detects protein interactions in vivo, the two-hybrid system, is described in detail for illustration only and not by way of limitation. One version of this system has been described (Chien et al., 1991, Proc. Natl. Acad. Sci. USA, 88:9578-9582) and is commercially available from Clontech (Palo Alto, Calif.).

Briefly, utilizing such a system, plasmids are constructed that encode two hybrid proteins: one consists of the DNA-binding domain of a transcription activator protein fused to a KSP interacting gene product and the other consists of the transcription activator protein's activation domain fused to an unknown protein that is encoded by a cDNA which has been recombined into this plasmid as part of a cDNA library. The DNA-binding domain fusion plasmid and the cDNA library are transformed into a strain of the yeast Saccharomyces cerevisiae that contains a reporter gene (e.g., HBS or lacZ) whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology may be used to screen activation domain libraries for proteins that interact with the “bait” gene product. By way of example, and not by way of limitation, KSP interacting gene products may be used as the bait gene product. Total genomic or cDNA sequences are fused to the DNA encoding an activation domain. This library and a plasmid encoding a hybrid of a bait KSP interacting gene product fused to the DNA-binding domain are cotransformed into a yeast reporter strain, and the resulting transformants are screened for those that express the reporter gene. For example, and not by way of limitation, a bait KSP interacting gene sequence, such as the coding sequence of a KSP interacting gene can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait KSP interacting gene product are to be detected can be made using methods routinely practiced in the art. According to the particular system described herein, for example, the cDNA fragments can be inserted into a vector such that they are translationally fused to the transcriptional activation domain of GALA. This library can be co-transformed along with the bait KSP interacting gene-GALA fusion plasmid into a yeast strain which contains a lacZ gene driven by a promoter which contains GALA activation sequence. A cDNA encoded protein, fused to GALA transcriptional activation domain, that interacts with bait KSP interacting gene product will reconstitute an active GALA protein and thereby drive expression of the HIS3 gene. Colonies which express HIS3 can be detected by their growth on petri dishes containing semi-solid agar based media lacking histidine. The cDNA can then be purified from these strains, and used to produce and isolate the bait KSP interacting gene-interacting protein using techniques routinely practiced in the art.

The interaction between a KSP interacting gene and its regulators may be determined by a standard method known in the art.

### 5.3.2. Methods of Screening for Agents

The invention provides methods for screening for agents that regulate the expression or modulate interaction of a KSP interacting protein, e.g., STK6 or TPX2, with other proteins or molecules.

### 5.3.2.1. Screening Assays

The following assays are designed to identify compounds that bind to a KSP interacting gene or gene products, bind to other cellular proteins that interact with a KSP interacting gene product, bind to cellular constituents, e.g., proteins, that are affected by a KSP interacting gene product, or bind to compounds that interfere with the interaction of the KSP interacting gene or gene product with other cellular proteins and to compounds which modulate the activity of a KSP interacting gene (i.e., modulate the level of STK6 or TPX2 gene expression and/or modulate the activity level of a STK6 or TPX2 gene product). Assays may additionally be utilized which identify compounds which bind to a KSP interacting gene regulatory sequences (e.g., promoter sequences), see e.g., Platt, K. A., 1994, J. Biol. Chem. 269:28558-28562, which is incorporated herein by reference in its entirety, which may modulate the level of expression of a KSP interacting gene. Compounds may include, but are not limited to, small organic molecules which are able to affect expression of the KSP interacting gene or some other gene involved in the pathways involving the KSP interacting gene, or other cellular proteins. Methods for the identification of such cellular proteins are described, above, in Section 5.3.1. Such cellular proteins may be involved in the regulation of the growth inhibitory effect of a KSP inhibitor. Further, among these compounds are compounds which affect the level of expression of a KSP interacting gene and/or activity of its gene product and which can be used in the regulation of resistance to the growth inhibitory effect of a KSP inhibitor.

Compounds may include, but are not limited to, peptides such as, for example, soluble peptides, including but not limited to, Ig-tailed fusion peptides, and members of random peptide libraries; (see, e.g., Lam, K. S. et al., 1991, Nature 354:82-84; Houghten, R. et al., 1991, Nature 354:84-86), and combinatorial chemistry-derived molecular library made of D- and/or L-configuration amino acids, phosphopeptides (including, but not limited to members of random or partially degenerate, directed phosphopeptide libraries; see, e.g., Songyang, Z. et al., 1993, Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope-binding fragments thereof), and small organic or inorganic molecules.

Compounds identified via assays such as those described herein may be useful, for example, in regulating the biological function of the KSP interacting gene product, and for ameliorating resistance to the growth inhibitory effect of a KSP inhibitor and/or enhancing the growth inhibitory effect of a KSP inhibitor. Assays for testing the effectiveness of compounds are discussed, below, in Section 5.3.2.2.

In vitro systems may be designed to identify compounds capable of binding the KSP interacting gene products of the invention. Compounds identified may be useful, for example, in modulating the activity of wild type and/or mutant of KSP interacting gene products, may be useful in elaborating the biological function of the KSP interacting gene product, may be utilized in screens for identifying compounds that disrupt normal KSP interacting gene product interactions, or may in themselves disrupt such interactions.

The principle of the assays used to identify compounds that bind to a KSP interacting gene product involves preparing a reaction mixture of the KSP interacting gene product and the test compound under conditions and for a time sufficient to allow the two components to interact and bind, thus forming a complex which can be removed and/or detected in the reaction mixture. These assays can be conducted in a variety of ways. For example, one method to conduct such an assay would involve anchoring the KSP interacting gene product or the test substance onto a solid phase and detecting the KSP interacting gene product/test compound complexes anchored on the solid phase at the end of the reaction. In one embodiment of such a method, the KSP interacting gene product may be anchored onto a solid surface, and the test compound, which is not anchored, may be labeled, either directly or indirectly.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for a KSP interacting gene product or the test compound to anchor any complexes formed in solution, and a labeled antibody specific for the other component of the possible complex to detect anchored complexes.

The KSP interacting gene or gene products may interact in vivo with one or more intracellular or extracellular molecules, such as proteins. Such molecules may include, but are not limited to, nucleic acid molecules and those proteins identified via methods such as those described, above, in Section 5.3.1. For purposes of this discussion, such molecules are referred to herein as “binding partners”. Compounds that disrupt the binding of a KSP interacting gene product may be useful in regulating the activity of the KSP interacting gene product. Compounds that disrupt the binding of a KSP interacting gene product may be useful in regulating the expression of the KSP interacting gene, such as by regulating the binding of a regulator of KSP interacting gene. Such compounds may include, but are not limited to molecules such as peptides, and the like, as described, for example, in Section 5.3.2.1. above, which would be capable of gaining access to the KSP interacting gene product.

The basic principle of the assay systems used to identify compounds that interfere with the interaction between a KSP interacting gene product and its intracellular or extracellular binding partner or partners involves preparing a reaction mixture containing the KSP interacting gene product, and the binding partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of the KSP interacting gene product and its binding partner. Control reaction mixtures are incubated without the test compound or with a placebo. The formation of any complexes between the KSP interacting protein and the binding partner is then detected. The formation of a complex in the control reaction, but not in the reaction mixture containing the test compound, indicates that the compound interferes with the interaction of the KSP interacting protein and the interactive binding partner. Additionally, complex formation within reaction mixtures containing the test compound and normal KSP interacting protein may also be compared to complex formation within reaction mixtures containing the test compound and a mutant KSP interacting protein. This comparison may be important in those cases wherein it is desirable to identify compounds that disrupt interactions of mutant but not normal KSP interacting proteins.

The assay for compounds that interfere with the interaction of the KSP interacting gene products and binding partners can be conducted in a heterogeneous or homogeneous format. Heterogeneous assays involve anchoring either the KSP interacting gene product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the KSP interacting gene products and the binding partners, e.g., by competition, can be identified by conducting the reaction in the presence of the test substance; i.e., by adding the test substance to the reaction mixture prior to or simultaneously with the KSP interacting protein and interactive binding partner. Alternatively, test compounds that disrupt preformed complexes, e.g. compounds with higher binding constants that displace one of the components from the complex, can be tested by adding the test compound to the reaction mixture after complexes have been formed. The various formats are described briefly below.

In a heterogeneous assay system, either the KSP interacting gene product or the interactive binding partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the KSP interacting gene product or binding partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds which inhibit complex or which disrupt preformed complexes can be identified.

In an alternate embodiment of the invention, a homogeneous assay can be used. In this approach, a preformed complex of the KSP interacting protein and the interactive binding partner is prepared in which either the KSP interacting gene product or its binding partners is labeled, but the signal generated by the label is quenched due to complex formation (see, e.g., U.S. Pat. No. 4,109,496 by Rubenstein which utilizes this approach for immunoassays). The addition of a test substance that competes with and displaces one of the species from the preformed complex will result in the generation of a signal above background. In this way, test substances which disrupt KSP interacting protein/binding partner interaction can be identified.

In a particular embodiment, the KSP interacting gene product can be prepared for immobilization using recombinant DNA techniques. For example, the coding region of a KSP interacting gene can be fused to a glutathione-5-transferase (GST) gene using a fusion vector, such as pGEX-5X-1, in such a manner that its binding activity is maintained in the resulting fusion protein. The interactive binding partner can be purified and used to raise a monoclonal antibody, using methods routinely practiced in the art. This antibody can be labeled with the radioactive isotope ¹²⁵I, for example, by methods routinely practiced in the art. In a heterogeneous assay, the GST fusion protein, e.g., the GST-STK6 or GST-TPX2 fusion protein, can be anchored to glutathione-agarose beads. The interactive binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between the KSP interacting protein and the interactive binding partner can be detected by measuring the amount of radioactivity that remains associated with the glutathione-agarose beads. A successful inhibition of the interaction by the test compound will result in a decrease in measured radioactivity.

Alternatively, the fusion protein, e.g., the GST-STK6 gene fusion protein, and the interactive binding partner can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the KSP interacting gene product/binding partner interaction can be detected by adding the labeled antibody and measuring the radioactivity associated with the beads.

In another embodiment of the invention, these same techniques can be employed using peptide fragments that correspond to the binding domains of the KSP interacting protein and/or the interactive binding partner (in cases where the binding partner is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described in this Section above, and allowed to interact with and bind to its labeled binding partner, which has been treated with a proteolytic enzyme, such as trypsin. After washing, a short, labeled peptide comprising the binding domain may remain associated with the solid material, which can be isolated and identified by amino acid sequencing. Also, once the gene coding for the binding partner is obtained, short gene segments can be engineered to express peptide fragments of the protein, which can then be tested for binding activity and purified or synthesized.

For example, and not by way of limitation, a STK6 or TPX2 gene product can be anchored to a solid material as described, above, in this Section by making a GST-STK6 or GST-TPX2 fusion protein and allowing it to bind to glutathione agarose beads. The interactive binding partner can be labeled with a radioactive isotope, such as ³⁵S, and cleaved with a proteolytic enzyme such as trypsin. Cleavage products can then be added to the anchored GST-STK6 or GST-TPX2 fusion protein and allowed to bind. After washing away unbound peptides, labeled bound material, representing the binding partner binding domain, can be eluted, purified, and analyzed for amino acid sequence by well-known methods. Peptides so identified can be produced synthetically or fused to appropriate facilitative proteins using recombinant DNA technology.

### 5.3.2.2. Screening Compounds that Regulate and/or Enhance the Growth Inhibitory Effect of a KSP Inhibitor

Any agents that regulate the expression of a KSP interacting gene and/or the interaction of a KSP interacting protein with its binding partners, e.g., compounds that are identified in Section 5.3.2.1., antibodies to a KSP interacting protein, and so on, can be further screened for its ability to regulate and/or enhance the growth inhibitory effect of a KSP inhibitor in cells. Any suitable proliferation or growth inhibition assays known in the art can be used for this purpose. In one embodiment, a candidate agent and a KSP inhibitor are applied to cells of a cell line, and a change in growth inhibitory effect is determined. Preferably, changes in growth inhibitory effect are determined using different concentrations of the candidate agent in conjunction with different concentrations of the KSPi such that one or more combinations of concentrations of the candidate agent and KSPi which cause 50% inhibition, i.e., the IC₅₀, are determined.

In a preferred embodiment, an MTT proliferation assay (see, e.g., van de Loosdrechet, et al., 1994, J. Immunol. Methods 174: 311-320; Ohno et al., 1991, J. Immunol. Methods 145:199-203; Ferrari et al., 1990, J. Immunol. Methods 131: 165-172; Alley et al., 1988, Cancer Res. 48: 589-601; Carmichael et al., 1987, Cancer Res. 47:936-942; Gerlier et al., 1986, J. Immunol. Methods 65:55-63; Mosmann, 1983, J. Immunological Methods 65:55-63) is used to screen for a candidate agent that can be used in conjunction with a KSPi to inhibit the growth of cells. The cells are treated with chosen concentrations of the candidate agent and a KSPi for 4 to 72 hours. The cells are then incubated with a suitable amount of 3-(4,5-dimethylthiazol-2-yl)-2,5-diphenyltetrazolium bromide (MTT) for 1-8 hours such that viable cells convert MTT into an intracellular deposit of insoluble formazan. After removing the excess MTT contained in the supernatant, a suitable MTT solvent, e.g., a DMSO solution, is added to dissolved the formazan. The concentration of MTT, which is proportional to the number of viable cells, is then measured by determining the optical density at 570 nm. A plurality of different concentrations of the candidate agent can be assayed to allow the determination of the concentrations of the candidate agent and the KSPi which causes 50% inhibition.

In another preferred embodiment, an alamarBlue™ Assay for cell proliferation is used to screen for a candidate agent that can be used in conjunction with a KSPi to inhibit the growth of cells (see, e.g., Page et al., 1993, Int. J. Oncol. 3:473-476). AlamarBlue assay is described in Section 5.2., supra. In specific embodiment, the alamarBlue™ assay is performed to determine whether transfection titration curves of an siRNA targeting a KSP interacting gene were changed by the presence of a KSPi of a chosen concentration, e.g., 25 nM of the KSP inhibitor (1S)-1-{[(2S)-4-(2,5-difluorophenyl)-2-phenyl-2,5-dihydro-1H-pyrrol-1-yl]carbonyl}-2-methylpropylamine. Cells were transfected with an STK6 siRNA. 4 hours after siRNA transfection, 100 microliter/well of DMEM/10% fetal bovine serum with or without the KSPi was added and the plates were incubated at 37° C. and 5% CO₂ for 68 hours. The medium was removed from the wells and replaced with 100 microliter/well DMEM/10% Fetal Bovine Serum (Invitrogen) containing 10% (vol/vol) alamarBlue™ reagent (Biosource International Inc., Camarillo, Calif.) and 0.001 volumes of 1M Hepes buffer tissue culture reagent (Invitrogen). The plates were incubated for 2 hours at 37° C. before they were read at 570 and 600 nm wavelengths on a SpectraMax plus plate reader (Molecular Devices, Sunnyvale, Calif.) using Softmax Pro 3.1.2 software (Molecular Devices). The percent reduced for wells transfected with a titration of STK6 siRNA with or without 25 nM (1S)-1-{[(2S)-4-(2,5-difluorophenyl)-2-phenyl-2,5-dihydro-1H-pyrrol-1-yl]carbonyl}-2-methylpropylamine were compared to luciferase siRNA-transfected wells. The number calculated for % Reduced for 0 nM luciferase siRNA-transfected wells without the KSPi was considered to be 100%.

### 5.3.2.3. Compounds Identified

The compounds identified in the screen include compounds that demonstrate the ability to selectively modulate the expression of a KSP interacting gene and regulate and/or enhance the growth inhibitory effect of a KSP inhibitor in cells. These compounds include but are not limited to siRNA, antisense, ribozyme, triple helix, antibody, and polypeptide molecules, aptamrs, and small organic or inorganic molecules.

The compounds identified in the screen also include compounds that modulate interaction of a KSP interacting with other proteins or molecules. In one embodiment, the compounds identified in the screen are compounds that modulate the interaction of a KSP interacting protein with its interaction partner. In another embodiment, the compounds identified in the screen are compounds that modulate the interaction of a KSP interacting gene with a transcription regulator.

### 5.3.3. Diagnostics

A variety of methods can be employed for the diagnostic and prognostic evaluation of cell or cells for their resistance to the growth inhibitory effect of a KSP inhibitor, e.g., (1S)-1-{[(2S)-4-(2,5-difluorophenyl)-2-phenyl-2,5-dihydro-1H-pyrrol-1-yl]carbonyl}-2-methylpropylamine, resulting from defective regulation of a KSP interacting gene, e.g., STK6 or TPX2, and for the identification of subjects having a predisposition to resistance to the growth inhibitory effect of a KSP inhibitor.

In one embodiment, the method comprises determining an expression level of a KSP interacting gene in the cell, in which an expression level above a predetermined threshold level indicates that the cell is KSPi resistant. Preferably, the predetermined threshold level is at least 2-fold, 4-fold, 8-fold, or 10-fold of the normal expression level of the KSP interacting gene. In another embodiment, the invention provides a method for evaluating KSPi resistance in a cell comprising determining a level of abundance of a protein encoded by a KSP interacting gene in the cell, in which a level of abundance of the protein above a predetermined threshold level indicates that the cell is KSPi resistant. In still another embodiment, the invention provides a method for evaluating KSPi resistance in a cell comprising determining a level of activity of a protein encoded by a KSP interacting gene in cells of the mammal, in which an activity level above a predetermined threshold level indicates that the cell is KSPi resistant. Preferably, the predetermined threshold level of abundance or activity is at least 2-fold, 4-fold, 8-fold, or 10-fold of the normal level of abundance or activity of the KSP interacting protein.

Such methods may, for example, utilize reagents such as the KSP interacting gene nucleotide sequences and antibodies directed against KSP interacting gene products, including peptide fragments thereof. Specifically, such reagents may be used, for example, for: (1) the detection of the presence of mutations in a KSP interacting gene, or the detection of either over- or under-expression of an mRNA of a KSP interacting gene relative to the normal expression level; and (2) the detection of either an over- or an under-abundance of a KSP interacting gene product relative to the normal level of a KSP interacting protein.

The methods described herein may be performed, for example, by utilizing pre-packaged diagnostic kits comprising at least one specific KSP interacting gene nucleic acid or anti-KSP interacting protein antibody reagent described herein, which may be conveniently used, e.g., in clinical settings, to diagnose patients exhibiting disorder or abnormalities related to a KSP interacting gene.

For the detection of mutations in a KSP interacting gene, any nucleated cell can be used as a starting source for genomic nucleic acid. For the detection of the expression of a KSP interacting gene or KSP interacting gene products, any cell type or tissue in which the KSP interacting gene is expressed may be utilized.

Nucleic acid-based detection techniques are described, below, in Section 5.3.3.1. Peptide detection techniques are described, below, in Section 5.3.3.2.

### 5.3.3.1. Detection of Expression of a KSP Interacting Gene

The expression of a KSP interacting gene, e.g., STK6 or TPX2, in cells or tissues, e.g., the cellular level of KSP interacting gene transcripts and/or the presence or absence of mutations, can be detected by utilizing a number of techniques. Nucleic acid from any nucleated cell can be used as the starting point for such assay techniques, and may be isolated according to standard nucleic acid preparation procedures which are well known to those of skill in the art. For example, the expression level of the KSP interacting gene can determined by measuring the expression level of the KSP interacting gene using one or more polynucleotide probes, each of which comprises a nucleotide sequence in the KSP interacting gene. In particularly preferred embodiments of the invention, the method is used to diagnose resistance of a cancer to a treatment using KSPi in a human.

DNA may be used in hybridization or amplification assays of biological samples to detect abnormalities involving the structure of a KSP interacting gene, including point mutations, insertions, deletions and chromosomal rearrangements. Such assays may include, but are not limited to, Southern analyses, single stranded conformational polymorphism analyses (SSCP), DNA microarray analyses, and PCR analyses.

Such diagnostic methods for the detection of KSP interacting gene-specific mutations can involve, for example, contacting and incubating nucleic acids including recombinant DNA molecules, cloned genes or degenerate variants thereof, obtained from a sample, e.g., derived from a patient sample or other appropriate cellular source, with one or more labeled nucleic acid reagents including recombinant DNA molecules, cloned genes or degenerate variants thereof, under conditions favorable for the specific annealing of these reagents to their complementary sequences within the KSP interacting gene. Preferably, the lengths of these nucleic acid reagents are at least 15 to 30 nucleotides. After incubation, all non-annealed nucleic acids are removed from the nucleic acid: KSP interacting gene molecule hybrid. The presence of nucleic acids which have hybridized, if any such molecules exist, is then detected. Using such a detection scheme, the nucleic acid from the cell type or tissue of interest can be immobilized, for example, to a solid support such as a membrane, or a plastic surface such as that on a microtiter plate or polystyrene beads. In this case, after incubation, non-annealed, labeled nucleic acid reagents are easily removed. Detection of the remaining, annealed, labeled KSP interacting gene nucleic acid reagents is accomplished using standard techniques well-known to those in the art. The KSP interacting gene sequences to which the nucleic acid reagents have annealed can be compared to the annealing pattern expected from a normal KSP interacting gene sequence in order to determine whether a KSP interacting gene mutation is present.

Alternative diagnostic methods for the detection of a KSP interacting gene specific nucleic acid molecules, in patient samples or other appropriate cell sources, may involve their amplification, e.g., by PCR (the experimental embodiment set forth in Mullis, K. B., 1987, U.S. Pat. No. 4,683,202), followed by the detection of the amplified molecules using techniques well known to those of skill in the art. The resulting amplified sequences can be compared to those which would be expected if the nucleic acid being amplified contained only normal copies of the KSP interacting gene in order to determine whether a KSP interacting gene mutation exists.

Among the nucleic acid sequences of a KSP interacting gene which are preferred for such hybridization and/or PCR analyses are those which will detect the presence of the KSP interacting gene splice site mutation.

Additionally, well-known genotyping techniques can be performed to identify individuals carrying a mutation in a KSP interacting gene. Such techniques include, for example, the use of restriction fragment length polymorphisms (RFLPs), which involve sequence variations in one of the recognition sites for the specific restriction enzyme used. Additionally, improved methods for analyzing DNA polymorphisms which can be utilized for the identification of mutations in a KSP interacting gene have been described which capitalize on the presence of variable numbers of short, tandemly repeated DNA sequences between the restriction enzyme sites. For example, Weber (U.S. Pat. No. 5,075,217, which is incorporated herein by reference in its entirety) describes a DNA marker based on length polymorphisms in blocks of (dC-dA)n-(dG-dT)n short tandem repeats. The average separation of (dC-dA)n-(dG-dT)n blocks is estimated to be 30,000-60,000 bp. Markers which are so closely spaced exhibit a high frequency co-inheritance, and are extremely useful in the identification of genetic mutations, such as, for example, mutations within the KSP interacting gene, and the diagnosis of diseases and disorders related to mutations in the KSP interacting.

Also, Caskey et al. (U.S. Pat. No. 5,364,759, which is incorporated herein by reference in its entirety) describe a DNA profiling assay for detecting short tri and tetra nucleotide repeat sequences. The process includes extracting the DNA of interest, such as the KSP interacting gene, amplifying the extracted DNA, and labelling the repeat sequences to form a genotypic map of the individual's DNA.

The expression level of a KSP interacting gene can also be assayed. For example, RNA from a cell type or tissue known, or suspected, to express the KSP interacting gene, such as a cancer cell type which exhibits KSPi resistance, may be isolated and tested utilizing hybridization or PCR techniques such as are described, above. The isolated cells can be derived from cell culture or from a patient. The analysis of cells taken from culture may be a necessary step in the assessment of cells to be used as part of a cell-based gene therapy technique or, alternatively, to test the effect of compounds on the expression of the KSP interacting gene. Such analyses may reveal both quantitative and qualitative aspects of the expression pattern of the KSP interacting gene, including activation or inactivation of the expression of the KSP interacting gene.

In one embodiment of such a detection scheme, a cDNA molecule is synthesized from an RNA molecule of interest (e.g., by reverse transcription of the RNA molecule into cDNA). A sequence within the cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method are chosen from among the KSP interacting gene nucleic acid reagents. The preferred lengths of such nucleic acid reagents are at least 9-30 nucleotides. For detection of the amplified product, the nucleic acid amplification may be performed using radioactively or non-radioactively labeled nucleotides. Alternatively, enough amplified product may be made such that the product may be visualized by utilizing any suitable nucleic acid staining method, e.g., by standard ethidium bromide staining.

Additionally, it is possible to perform such KSP interacting gene expression assays “in situ”, i.e., directly upon tissue sections (fixed and/or frozen) of patient tissue obtained from biopsies or resections, such that no nucleic acid purification is necessary. Nucleic acids from a KSP interacting gene may be used as probes and/or primers for such in situ procedures (see; for example, Nuovo, G. J., 1992, “PCR In Situ Hybridization: Protocols And Applications”, Raven Press, NY).

Alternatively, if a sufficient quantity of the appropriate cells can be obtained, standard Northern analysis can be performed to determine the level of mRNA expression of the KSP interacting gene.

The expression of KSP interacting gene in cells or tissues, e.g., the cellular level of KSP interacting transcripts and/or the presence or absence of mutations, can also be evaluated using DNA microarray technologies. In such technologies, one or more polynucleotide probes each comprising a sequence of the KSP interacting gene are used to monitor the expression of the KSP interacting gene. The present invention therefore provides DNA microarrays comprising polynucleotide probes comprising sequences of the KSP interacting gene.

Any formats of DNA microarray technologies can be used in conjunction with the present invention. In one embodiment, spotted cDNA arrays are prepared by depositing PCR products of cDNA fragments, e.g., full length cDNAs, ESTs, etc., of the KSP interacting gene onto a suitable surface (see, e.g., DeRisi et al., 1996, Nature Genetics 14:457-460; Shalon et al., 1996, Genome Res. 6:689-645; Schena et al., 1995, Proc. Natl. Acad. Sci U.S.A. 93:10539-11286; and Duggan et al., Nature Genetics Supplement 21:10-14). In another embodiment, high-density oligonucleotide arrays containing oligonucleotides complementary to sequences of the KSP interacting gene are synthesized in situ on the surface by photolithographic techniques (see, e.g., Fodor et al., 1991, Science 251:767-773; Pease et al., 1994, Proc. Natl. Acad. Sci. U.S.A. 91:5022-5026; Lockhart et al., 1996, Nature Biotechnology 14:1675; McGall et al., 1996, Proc. Natl. Acad. Sci. U.S.A. 93:13555-13560; U.S. Pat. Nos. 5,578,832; 5,556,752; 5,510,270; 5,858,659; and 6,040,138). This format of microarray technology is particular useful for detection of single nucleotide polymorphisms (SNPs) (see, e.g., Hacia et al., 1999, Nat Genet. 22:164-7; Wang et al., 1998, Science 280:1077-82). In yet another embodiment, high-density oligonucleotide arrays containing oligonucleotides complementary to sequences of the KSP interacting gene are synthesized in situ on the surface by inkjet technologies (see, e.g., Blanchard, International Patent Publication WO 98/41531, published Sep. 24, 1998; Blanchard et al., 1996, Biosensors and Bioelectronics 11:687-690; Blanchard, 1998, in Synthetic DNA Arrays in Genetic Engineering, Vol. 20, J. K. Setlow, Ed., Plenum Press, New York at pages 111-123). In still another embodiment, DNA microarrays that allow electronic stringency control can be used in conjunction with polynucleotide probes comprising sequences of the KSP interacting gene (see, e.g., U.S. Pat. No. 5,849,486).

### 5.3.3.2. Detection of KSP Interacting Gene Products

Antibodies directed against wild type or mutant KSP interacting gene products or conserved variants or peptide fragments thereof may be used as diagnostics and prognostics of KSPi resistance, as described herein. Such diagnostic methods may be used to detect abnormalities in the expression level of a KSP interacting gene, or abnormalities in the structure and/or temporal, tissue, cellular, or subcellular location of a KSP interacting gene product.

Because KSP interacting gene products are intracellular gene products, the antibodies and immunoassay methods described below have important in vitro applications in assessing the efficacy of treatments for disorders resulting from defective regulation of KSP interacting gene such as proliferative diseases. Antibodies, or fragments of antibodies, such as those described below, may be used to screen potentially therapeutic compounds in vitro to determine their effects on KSP interacting gene expression and KSP interacting peptide production. The compounds which have beneficial effects on disorders related to defective regulation of KSP interacting can be identified, and a therapeutically effective dose determined.

In vitro immunoassays may also be used, for example, to assess the efficacy of cell-based gene therapy for disorders related to defective regulation of a KSP interacting gene. Antibodies directed against KSP interacting peptides may be used in vitro to determine the level of KSP interacting gene expression achieved in cells genetically engineered to produce KSP interacting peptides. Given that evidence disclosed herein indicates that the KSP interacting gene product is an intracellular gene product, such an assessment is, preferably, done using cell lysates or extracts. Such analysis will allow for a determination of the number of transformed cells necessary to achieve therapeutic efficacy in vivo, as well as optimization of the gene replacement protocol.

The tissue or cell type to be analyzed will generally include those which are known, or suspected, to express the KSP interacting gene, such as, a KSPi resistant cancer cell type. The protein isolation methods employed herein may, for example, be such as those described in Harlow and Lane (Harlow, E. and Lane, D., 1988, “Antibodies: A Laboratory Manual”, Cold Spring Harbor Laboratory Press, Cold Spring Harbor, N.Y.), which is incorporated herein by reference in its entirety. The isolated cells can be derived from cell culture or from a patient. The analysis of cells taken from culture may be used to test the effect of compounds on the expression of the KSP interacting gene.

Preferred diagnostic methods for the detection of KSP interacting gene products or conserved variants or peptide fragments thereof, may involve, for example, immunoassays wherein the KSP interacting gene products or conserved variants or peptide fragments are detected by their interaction with an anti-KSP interacting gene product-specific antibody.

For example, antibodies, or fragments of antibodies, that bind a KSP interacting protein, may be used to quantitatively or qualitatively detect the presence of KSP interacting gene products or conserved variants or peptide fragments thereof. This can be accomplished, for example, by immunofluorescence techniques employing a fluorescently labeled antibody (see below, this Section) coupled with light microscopic, flow cytometric, or fluorimetric detection. Such techniques are especially preferred if such KSP interacting gene products are expressed on the cell surface.

The antibodies (or fragments thereof) useful in the present invention may, additionally, be employed histologically, as in immunofluorescence or immunoelectron microscopy, for in situ detection of KSP interacting gene products or conserved variants or peptide fragments thereof. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the KSP interacting gene product, or conserved variants or peptide fragments, but also its distribution in the examined tissue. Using the present invention, those of ordinary skill will readily perceive that any of a wide variety of histological methods (such as staining procedures) can be modified in order to achieve such in situ detection.

Immunoassays for KSP interacting gene products or conserved variants or peptide fragments thereof will typically comprise incubating a sample, such as a biological fluid, a tissue extract, freshly harvested cells, or lysates of cells which have been incubated in cell culture, in the presence of a detectably labeled antibody capable of identifying KSP interacting gene products or conserved variants or peptide fragments thereof, and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled KSP interacting protein specific antibody. The solid phase support may then be washed with the buffer a second time to remove unbound antibody. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tub, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of anti-KSP interacting gene product antibody may be determined according to well known methods. Those skilled in the art will be able to determine operative and optimal assay conditions for each determination by employing routine experimentation.

One of the ways in which the KSP interacting gene peptide-specific antibody can be detectably labeled is by linking the same to an enzyme and use in an enzyme immunoassay (EIA) (Voller, A., “The Enzyme Linked Immunosorbent Assay (ELISA)”, 1978, Diagnostic Horizons 2:1-7, Microbiological Associates Quarterly Publication, Walkersville, Md.); Voller, A. et al., 1978, J. Clin. Pathol. 31:507-520; Butler, J. E., 1981, Meth. Enzymol. 73:482-523; Maggio, E. (ed.), 1980, Enzyme Immunoassay, CRC Press, Boca Raton, Fla.,; Ishikawa, E. et al., (eds.), 1981, Enzyme Immunoassay, Kgaku Shoin, Tokyo). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect KSP interacting peptides through the use of a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

### 5.3.4. Methods of Regulating Expression of KSP Interacting Genes

A variety of therapeutic approaches may be used in accordance with the invention to modulate expression of a KSP interacting gene, e.g., STK6 or TPX2, in vivo. For example, siRNA molecules may be engineered and used to silence the KSP interacting gene in vivo. Antisense DNA molecules may also be engineered and used to block translation of a KSP interacting mRNA in vivo. Alternatively, ribozyme molecules may be designed to cleave and destroy the KSP interacting mRNAs in vivo. In another alternative, oligonucleotides designed to hybridize to the 5′ region of the KSP interacting gene (including the region upstream of the coding sequence) and form triple helix structures may be used to block or reduce transcription of the KSP interacting gene. If desired, oligonucleotides can also be designed to hybridize and form triple helix structures with the binding site of a negative regulator so as to block the binding of the negative regulator and to enhance the transcription of the KSP interacting gene.

In a preferred embodiment, siRNA, antisense, ribozyme, and triple helix nucleotides are designed to inhibit the translation or transcription of one or more KSP interacting protein isoforms with minimal effects on the expression of other genes that may share one or more sequence motif with the KSP interacting gene. To accomplish this, the oligonucleotides used should be designed on the basis of relevant sequences unique to the KSP interacting gene.

For example, and not by way of limitation, the oligonucleotides should not fall within those region where the nucleotide sequence of a KSP interacting gene is most homologous to that of the other genes. In the case of antisense molecules, it is preferred that the sequence be chosen from the list above. It is also preferred that the sequence be at least 18 nucleotides in length in order to achieve sufficiently strong annealing to the target mRNA sequence to prevent translation of the sequence. Izant et al., 1984, Cell, 36:1007-1015; Rosenberg et al., 1985, Nature, 313:703-706.

In the case of the “hammerhead” type of ribozymes, it is also preferred that the target sequences of the ribozymes be chosen from the list above. Ribozymes are RNA molecules which possess highly specific endoribonuclease activity. Hammerhead ribozymes comprise a hybridizing region which is complementary in nucleotide sequence to at least part of the target RNA, and a catalytic region which is adapted to cleave the target RNA. The hybridizing region contains nine (9) or more nucleotides. Therefore, the hammerhead ribozymes of the present invention have a hybridizing region which is complementary to the sequences listed above and is at least nine nucleotides in length. The construction and production of such ribozymes is well known in the art and is described more fully in Haseloff et al., 1988, Nature, 334:585-591.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO 88/04300 by University Patents Inc.; Been et al., 1986, Cell, 47:207-216). The Cech endoribonucleases have an eight base pair active site which hybridizes to a target RNA sequence whereafter cleavage of the target RNA takes place.

In the case of oligonucleotides that hybridize to and form triple helix structures at the 5′ terminus of the KSP interacting gene and can be used to block transcription, it is preferred that they be complementary to those sequences in the 5′ terminus of KSP interacting gene which are not present in the other genes whose expression level is not to be affected. It is also preferred that the sequences do not include those regions of the promoter of a KSP interacting gene which are even slightly homologous to that of such other genes. The foregoing compounds can be administered by a variety of methods which are known in the art including, but not limited to the use of liposomes as a delivery vehicle. Naked DNA or RNA molecules may also be used where they are in a form which is resistant to degradation such as by modification of the ends, by the formation of circular molecules, or by the use of alternate bonds including phosphothionate and thiophosphoryl modified bonds. In addition, the delivery of nucleic acid may be by facilitated transport where the nucleic acid molecules are conjugated to poly-lysine or transferrin. Nucleic acid may also be transported into cells by any of the various viral carriers, including but not limited to, retrovirus, vaccinia, AAV, and adenovirus.

Alternatively, a recombinant nucleic acid molecule which encodes, or is, such antisense, ribozyme, triple helix, or KSP interacting gene nucleic acid molecule can be constructed. This nucleic acid molecule may be either RNA or DNA. If the nucleic acid encodes an RNA, it is preferred that the sequence be operatively attached to a regulatory element so that sufficient copies of the desired RNA product are produced. The regulatory element may permit either constitutive or regulated transcription of the sequence. In vivo, that is, within the cells or cells of an organism, a transfer vector such as a bacterial plasmid or viral RNA or DNA, encoding one or more of the RNAs, may be transfected into cells e.g. (Llewellyn et al., 1987, J. Mol. Biol., 195:115-123; Hanahan et al. 1983, J. Mol. Biol., 166:557-580). Once inside the cell, the transfer vector may replicate, and be transcribed by cellular polymerases to produce the RNA or it may be integrated into the genome of the host cell. Alternatively, a transfer vector containing sequences encoding one or more of the RNAs may be transfected into cells or introduced into cells by way of micromanipulation techniques such as microinjection, such that the transfer vector or a part thereof becomes integrated into the genome of the host cell.

RNAi can also be used to knock down the expression of a KSP interacting gene. In one embodiment, double-stranded RNA molecules of 21-23 nucleotides which hybridize to a homologous region of mRNAs transcribed from the KSP interacting gene are used to degrade the mRNAs, thereby “silence” the expression of the KSP interacting gene. Preferably, the dsRNAs have a hybridizing region, e.g., a 19-nucleotide double-stranded region, which is complementary to a sequence of the coding sequence of the KSP interacting gene. Any siRNA targeting an appropriate coding sequence of a KSP interacting gene, e.g., a human STK6 or TPX2 gene, can be used in the invention. As an exemplary embodiment, 21-nucleotide double-stranded siRNAs targeting the coding regions of KSP interacting gene are designed according to standard selection rules (see, e.g., Elbashir et al., 2002, Methods 26:199-213, which is incorporated herein by reference in its entirety).

Any standard method for introducing siRNAs into cells can be used. In one embodiment, gene silencing is induced by presenting the cell with the siRNA targeting the KSP interacting gene (see, e.g., Elbashir et al., 2001, Nature 411, 494-498; Elbashir et al., 2001, Genes Dev. 15, 188-200, all of which are incorporated by reference herein in their entirety). The siRNAs can be chemically synthesized, or derived from cleavage of double-stranded RNA by recombinant Dicer. Another method to introduce a double stranded DNA (dsRNA) for silencing of the KSP interacting gene is shRNA, for short hairpin RNA (see, e.g., Paddison et al., 2002, Genes Dev. 16, 948-958; Brummelkamp et al., 2002, Science 296, 550-553; Sui, G. et al. 2002, Proc. Natl. Acad. Sci. USA 99, 5515-5520, all of which are incorporated by reference herein in their entirety). In this method, an siRNA targeting a KSP interacting gene is expressed from a plasmid (or virus) as an inverted repeat with an intervening loop sequence to form a hairpin structure. The resulting RNA transcript containing the hairpin is subsequently processed by Dicer to produce siRNAs for silencing. Plasmid-based shRNAs can be expressed stably in cells, allowing long-term gene silencing in cells both in vitro and in vivo (see, McCaffrey et al. 2002, Nature 418, 38-39; Xia et al., 2002, Nat. Biotech. 20, 1006-1010; Lewis et al., 2002, Nat. Genetics 32, 107-108; Rubinson et al., 2003, Nat. Genetics 33, 401-406; Tiscomia et al., 2003, Proc. Natl. Acad. Sci. USA 100, 1844-1848, all of which are incorporated by reference herein in their entirety). SiRNAs targeting the KSP interacting gene can also be delivered to an organ or tissue in a mammal, such a human, in vivo (see, e.g., Song et al. 2003, Nat. Medicine 9, 347-351; Sorensen et al., 2003, J. Mol. Biol. 327, 761-766; Lewis et al., 2002, Nat. Genetics 32, 107-108, all of which are incorporated by reference herein in their entirety). In this method, a solution of siRNA is injected intravenously into the mammal. The siRNA can then reach an organ or tissue of interest and effectively reduce the expression of the target gene in the organ or tissue of the mammal.

### 5.3.5. Methods of Regulating Activity of a KSP Interacting Protein and/or Its Pathways

The activity of a KSP interacting protein can be regulated by modulating the interaction of the KSP interacting protein with its binding partners. In one embodiment, agents, e.g., antibodies, aptamers, small organic or inorganic molecules, can be used to inhibit binding of such a binding partner such that KSPi resistance is regulated. In another embodiment, agents, e.g., antibodies, aptamers, small organic or inorganic molecules, can be used to inhibit the activity of a protein in a KSP interacting protein regulatory pathway such that KSPi resistance is regulated.

### 5.3.6. Cancer Therapy by Targeting KSP Interacting Gene and/or Gene Product

The methods and/or compositions described above for modulating expression and/or activity of a KSP interacting gene or protein, e.g., STK6 or TPX2 gene or protein, may be used to treat patients who have a cancer in conjunction with a KSPi. In particular, the methods and/or compositions may be used in conjunction with a KSPi for treatment of a patient having a cancer which exhibits the KSP interacting gene or protein mediated KSPi resistance. Such therapies may be used to treat cancers, including but not limted to, rhabdomyosarcoma, neuroblastoma and glioblastoma, small cell lung cancer, osteoscarcoma, pancreatic cancer, breast and prostate cancer, murine melanoma and leukemia, and B-cell lymphoma.

In preferred embodiments, the methods and/or compositions of the invention are used in conjunction with a KSPi for treatment of a patient having a cancer which exhibits STK6 or TPX2 mediated KSPi resistance. In such embodiments, the expression and/or activity of STK6 or TPX2 are modulated to confer cancer cells sensitivity to a KSPi, thereby conferring or enhancing the efficacy of KSPi therapy.

In a combination therapy, one or more compositions of the present invention can be administered before, at the same time of, or after the administration of a KSPi. In one embodiment, the compositions of the invention are administered before the administration a KSPi. The time intervals between the administration of the compositions of the invention and a KSPi can be determined by routine experiments that are familiar to one skilled person in the art. In one embodiment, a KSPi is given after the KSP interacting protein level reaches a desirable threshold. The level of KSP interacting protein can be determined by using any techniques described supra.

In another embodiment, the compositions of the invention are administered at the same time with the KSPi.

In still another embodiment, one or more of the compositions of the invention are also administered after the administration of a KSPi. Such administration can be beneficial especially when the KSPi has a longer half life than that of the one or more compositions of the invention used in the treatment.

It will be apparent to one skilled person in the art that any combination of different timing of the administration of the compositions of the invention and a KSPi can be used. For example, when the KSPi has a longer half life than that of the composition of the invention, it is preferable to administer the compositions of the invention before and after the administration of the KSPi.

The frequency or intervals of administration of the compositions of the invention depends on the desired level of the KSP interacting protein, which can be determined by any of the techniques described supra. The administration frequency of the compositions of the invention can be increased or decreased when the KSP interacting protein level changes either higher or lower from the desired level.

The effects or benefits of administration of the compositions of the invention alone or in conjunction with a KSPi can be evaluated by any methods known in the art, e.g., by methods that are based on measuring the survival rate, side effects, dosage requirement of the KSPi, or any combinations thereof. If the administration of the compositions of the invention achieves any one or more of the benefits in a patient, such as increasing the survival rate, decreasing side effects, lowing the dosage requirement for the KSPi, the compositions of the invention are said to have augmented the KSPi therapy, and the method is said to have efficacy.

### 5.3.7. Cancer Therapy by Targeting STK6 Gene in Combination with Other Drugs that Target Mitosis

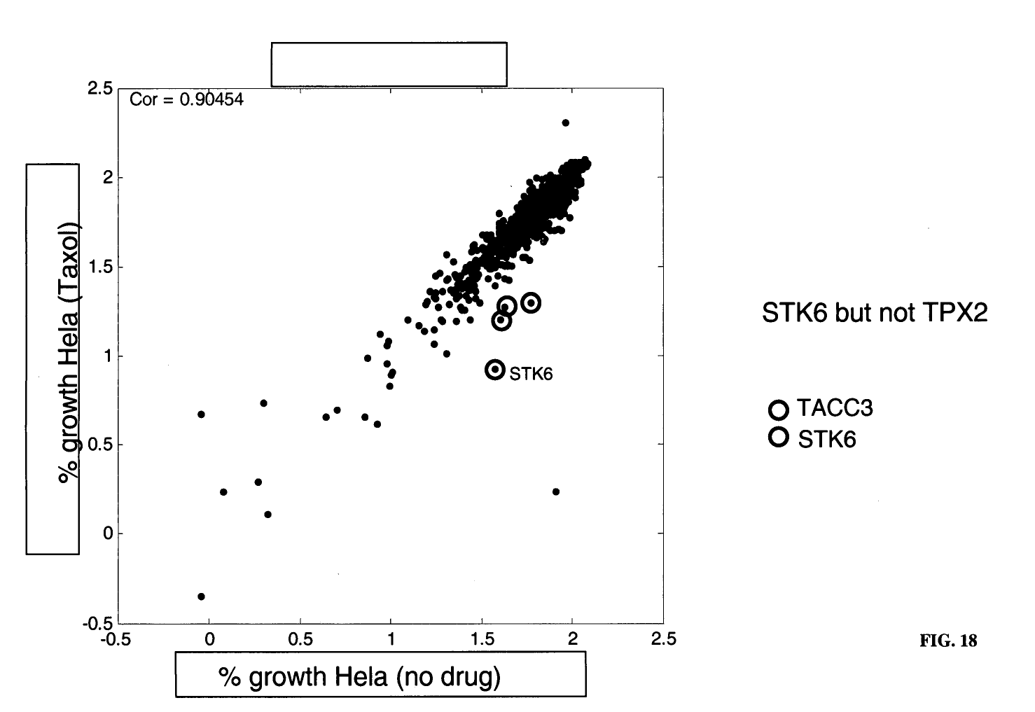

The inventors have also discovered that STK6 also interacts with other drugs that target mitosis, e.g., taxol. FIG. 18 shows that STK6 sensitize HeLa cells to taxol treatment. Thus, the invention also provides methods and compositions described above for modulating STK6 expression and/or activity for treating patients who have a cancer in conjunction with a drug that targets mitosis, e.g., taxol. In particular, the methods and/or compositions may be used in conjunction with taxol for treatment of a patient having a cancer which exhibits STK6-mediated taxol resistance. Such therapies may be used to treat cancers, including but not limted to, rhabdomyosarcoma, neuroblastoma and glioblastoma, small cell lung cancer, osteoscarcoma, pancreatic cancer, breast and prostate cancer, murine melanoma and leukemia, and B-cell lymphoma.

### 5.4. Genes and Gene Products Interacting with a DNA Damaging Agent and Their Uses

The invention provides methods and compositions for utilizing the genes and gene products that interact with DNA damaging agents in treating diseases. Such a gene is often referred to as a “DNA damage response gene.” A gene product, e.g., a protein, encoded by such a gene is often referred to as a “DNA damage response gene product.” The invention also provides methods and compositions for utilizing these genes and their products for screening for agents that regulate the expression/activity of the genes/gene products, and/or modulating interaction of the genes or proteins with other proteins or molecules. The invention further provides methods and compositions for utilizing these genes and gene products for screening for agents that are useful in regulating sensitivity of cells to the growth inhibitory effect of DNA damaging agents and/or in enhancing the growth inhibitory effect of DNA damaging agent in a cell or organism. The invention also provides methods and compositions for utilizing these gene and gene products for diagnosing resistance or sensitivity to the growth inhibitory effect of DNA damaging agents, and for treatment of diseases in conjunction with a therapy using one or more DNA damaging agents.

### 5.4.1. Genes and Gene Products Interacting with a DNA Damaging Agent

The invention provides genes that are capable of reducing or enhancing cell killing by DNA damaging agents. These genes can be used in conjunction with the DNA damaging agents described in Section 5.4.2., infra. Uses of these genes are described in Sections 5.4.3 and 5.4.4., infra.

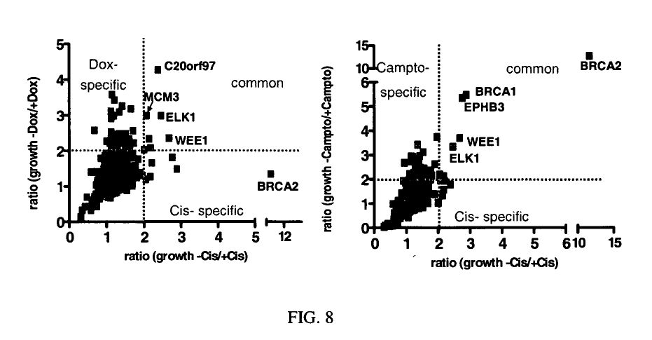

In one embodiment, the invention provides genes that are capable of reducing or enhancing cell killing by a DNA damaging agent, e.g., cis, dox, or campto, by at least 1.5 fold, 1.6 fold, 1.7 fold, 1.8 fold, and 1.9 fold. In a preferred embodiment, the invention provides the following genes whose silencing enhances cell killing by a DNA damaging agent by at least 2.0 fold: BRCA2, EPHB3, WEE1, and ELK1. FIG. 8 shows that silencing of BRCA2, EPHB3, WEE1, and ELK1 enhances cell killing due to a DNA damaging agent by at least 2 fold. The invention provides method of treatment of cancer by regulating, e.g., enhancing or reducing, the expression of such genes and/or activity of a protein encoded by such genes, in conjunction with a therapy involving administration of a DNA damaging agent.

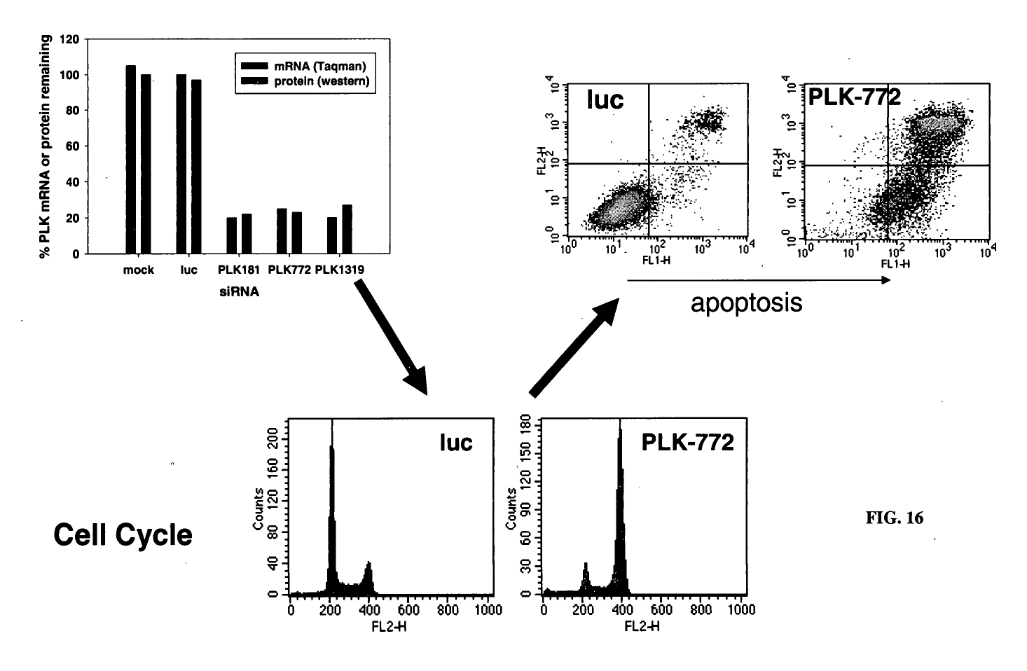

The invention also provides genes that are capable of reducing or enhancing cell killing by a particular type of DNA damaging agents. Table IIA shows genes whose silencing enhances or reduces cell killing by a DNA binding agent such as DNA groove binding agent, e.g., DNA minor groove binding agent; DNA crosslinking agent; intercalating agent; and DNA adduct forming agent. In one embodiment, the invention provides genes whose silencing enhances cell killing by a DNA binding agent, e.g., cis, by at least 1.5 fold, 1.6 fold, 1.7 fold, 1.8 fold, and 1.9 fold as listed in Table IIA, e.g., gene IDs 752-806 (1.5 fold), gene IDs 771-806 (1.6 fold), gene IDs 784-806 (1.7 fold), gene IDs 789-806 (1.8 fold), and gene IDs 793-806 (1.9 fold). In a preferred embodiment, the invention provides following genes whose silencing enhances cell killing by a DNA binding agent, e.g., cis, by at least 2 fold: BRCA1, BRCA2, EPHB3, WEE1, ELK1, RPS6KA6, BRAF, GPRK6, MCM3, CDC42, KIF2C, CENPE, CDC25B, and C20orf97. In another embodiment, the invention provides following genes whose silencing reduces cell killing by a DNA binding agent, e.g., cis, by at least 2 fold: PLK (see FIG. 16). The invention provides method of treatment of cancer by regulating, e.g., enhancing or reducing, the expression of such genes and/or activity of a protein encoded by such genes, in conjunction with a therapy involving administration of a DNA binding agent.

The invention also provides genes that are capable of reducing or enhancing cell killing by Topo I inhibitor, such as camptothecin. In one embodiment, the invention provides genes whose silencing enhances cell killing by a topo I inhibitor, e.g., campto, by at least 1.5 fold, 1.6 fold, 1.7 fold, 1.8 fold, and 1.9 fold as listed in Table IIB, e.g., gene IDs 635-807 (1.5 fold), gene IDs 673-807 (1.6 fold), gene IDs 702-807 (1.7 fold), gene IDs 727-807 (1.8 fold), and gene IDs 749-807 (1.9 fold). In a preferred embodiment, the invention provides genes whose silencing enhances cell killing by a Topo I inhibitor, e.g., campto, by at least 2 fold, e.g., NM_{—}139286, TOP3B, WASL, STAT4, CHEK1, BCL2, NM_{—}016263, TOP2B, TGFBR1, MAPK8, RHOK, NM_{—}017719, TERT, ANAPC5, NM_{—}021170, SGK2, C20orf97, CSF1R, EGR2, AATK, TCF3, CDC45L, STAT3, PRKY, BMPR1B, KIF2C, PTTG1, NM_{—}019089, FOXO1A, STK4, SRC, ELK₁, NM_{—}018492, RASA2, GPRK6, BLK, ABL1, HSPCB, PRKACA, CCNE2, CTNNBIP1, NM_{—}013367, FRAT1, PIK3C2A, NM_{—}017769, XM_{—}170783, NM_{—}016457, XM_{—}064050, STK6, RALBP1, ELK1, NF1, STAT5A, WEE1, PTK6, RPS6KA6, BRCA1, EPHB3, and BRCA2. In another preferred embodiment, the invention provides genes whose silencing enhances cell killing by a Topo I inhibitor, e.g., campto, by at least 3 fold, e.g., XM_{—}064050, STK6, RALBP1, ELK1, NF1, STAT5A, WEE1, PTK6, RPS6KA6, BRCA1, EPHB3, and BRCA2. In another embodiment, the invention provides genes whose silencing reduces cell killing by a topo I inhibitor, e.g., campto, by at least 2 fold, e.g., PLK, CCNA2, MADH4, NFKB1, RRM2B, TSG101, DCK, CDC5L, CDCA8, NM_{—}006101, INSR. The invention provides method of treatment of cancer by regulating, e.g., enhancing or reducing, the expression of such genes and/or activity of a protein encoded by such genes, in conjunction with a therapy involving administration of a Topo I inhibitor.

The invention also provides genes that are capable of reducing or enhancing cell killing by Topo II inhibitor, such as doxorubicin. In one embodiment, the invention provides genes whose silencing enhances cell killing by a DNA binding agent, e.g., dox, by at least 1.5 fold, 1.6 fold, 1.7 fold, 1.8 fold, and 1.9 fold as listed in Table IIC, e.g., gene IDs 657-830 (1.5 fold), gene IDs 685-830 (1.6 fold), gene IDs 723-830 (1.7 fold), gene IDs 750-830 (1.8 fold), and gene IDs 767-830 (1.9 fold). In a preferred embodiment, the invention provides genes whose silencing enhances cell killing by a Topo II inhibitor, e.g., dox, by at least 2 fold, e.g., PTK2, KRAS2, BRA, FZD4, RASAL2, CENPE, CCNH, MAP4K3, MAP4K2, ERBB3, RHOK, MYO3A, AXIN1, INPP5D, NM_{—}018401, NEK1, TGFBR1, XM_{—}064050, STAT4, MAP3K1, CCNE2, STK6, HDAC4, CTNNA1, EIF4EBP1, ACVR2B, CDC42, MAPK8, BLK, WEE1, KIF26A, TCF1, NM_{—}019089, NOTCH4, HDAC3, PIK3CB, CCNG2, TLK2, XM_{—}066649, MCM3, ELK1, PTK6, ABL1, FZD4, XM_{—}170783, CHUK, SRC, NM_{—}016263, and C20orf97. In another preferred embodiment, the invention provides genes whose silencing enhances cell killing by a Topo II inhibitor, e.g., dox, by at least 3 fold, e.g., ELK1, PTK6, ABL1, FZD4, XM_{—}170783, CHUK, SRC, NM_{—}016263, and C20orf97. In another embodiment, the invention provides genes whose silencing reduces cell killing by a Topo II inhibitor, e.g., dox, by at least 2 fold, e.g., PLK (see FIG. 16). The invention provides method of treatment of cancer by regulating, e.g., enhancing or reducing, the expression of such genes and/or activity of a protein encoded by such genes, in conjunction with a therapy involving administration of a Topo II inhibitor.

In a preferred embodiment, the invention provides CHEK1, BRCA1, BARD1, and RAD51 as genes that are capable of enhancing killing of p53− cells by DNA damaging agents.

In another preferred embodiment, the invention provides WEE1 as a gene that is capable of reducing or enhancing cell killing by DNA damaging agents. Wee1 is a negative mitosis regulator protein first identified in fission yeast Schizosaccharmomyces pombe (Russell and Nurse, 1987 Cell 49:559-67). Wee1⁻ mutants have a short G2 period and enter mitosis at half the size (hence the name wee) of wild type cells. In cells that overexpress cdc25, a mitotic inducer, wee1 activity is required to prevent lethality by premature mitosis (mitotic catastrophe). The human homolog of wee1 was cloned by transcomplementation of a S. pombe temperature-dependent wee1⁻¹, cdc25 over-expressing mutant (Igarashi et al., 1991, Nature 353:80-83). Overexpression of the human wee1 in fission yeast generates elongated cells from inhibition of the G2-M transition of the cell cycle. This human Wee1 clone was significantly smaller than its yeast counterpoint, and was later found to be missing a portion of the amino terminus sequence (Watanabe et al., 1995, EMBO 14:1878-91).

The single copy human wee1 gene is located on chromosome 11 (Taviaux and Demaille, 1993, Genomics 15:194-196). The wee1 gene is 16.96 kb with 11 exons, encoding a 4.23 kb mRNA transcript. The 94 kDa human Wee1 protein comprises 646 amino acids. According to Aceview, an integrated analysis of publicly available experimental cDNA data (http://www.ncbi.nlm.nih.gov/IEB/Research/Acembly/av.cgi?c=locusid&org=9606&1=7465) there may be six smaller Wee1 protein isoforms produced by alternative splicing. Wee1 expression has been found in wide range of human cells, such as lung fibroblasts, embryonic fibroblasts, cervical cancer HeLa cells, colon adenocarcinoma, bladder carcinoma (Igarashi et al., 1991, Nature 353:80-83), uterine, blood vessel, liver, eye, spleen, gall bladder, skin, cartilage, and various tumor cell lines (UniGene, http://www.ncbi.nlm.nih.gov/UniGene/). Wee1-like proteins have also been identified in mouse, rat, C. elegans, Drosphila, and S. cerevisiae, with the mouse and rat 646 amino acid proteins having the highest degree of similarity (89% and 91% respectively) (UniGene). Full-length human Wee1 sequence has five stretches with high PEST scores, and the catalytic kinase domain is in the C-terminus (Watanabe et al., 1995, EMBO 14:1878-91). The conserved Lys114 residue appears to be critical for Wee1 kinase activity (McGowan and Russell, 1993, EMBO 12:75-85).

Other Wee1-related kinases have been identified in multiple species. Xenopus Wee1 is expressed maternally (oocytes), while Wee2 is expressed in zygotes in non-dividing tissue. In vertebrates, the related Myt1 has similar phosphorylating activity to Wee1 (reviewed in Kellogg, 2003, J. Cell Sci. 116:4883-4890). A Wee1B has also been identified in humans, which is almost exclusively expressed in mature oocytes (Nakanishi et al., 2000, Genes to Cells 5:839-847).

Wee1 is a nuclear tyrosine kinase belonging to the family of Ser/Thr family of protein kinases. Wee1 ensures the completion of DNA replication prior to mitosis by inhibiting Cdc2-cyclin B kinase at the G2/M transition of the cell cycle. Phosphorylation of the Thr14 and Tyr15 residues in the ATP-binding site of Cdc2 inhibits its activity; Wee1 tyrosine kinase phosphorylates the Tyr15 residue at the N-terminus. A second related protein kinase, Mik1 (Myt1), phosphorylates Cdc2 on both Thr14 and Tyr15. Cdc2 activity is required for progression into mitosis. Dephosphorylation of the critical Tyr15 residue is catalyzed by Cdc25, functioning in opposition to Wee1. Balance of Wee1 and Cdc25 activities determines entry into mitosis (reviewed in Kellogg, 2003, J. Cell Sci. 116:4883-4890; Pendergast, 1996, Curr. Opin. Cell Biol. 8:174-181).

Wee1 activity is highly regulated during the cell cycle. During S and G2 phases, Wee1 activity increases, paralleling increases in protein levels. Wee1 activity is suppressed at mitosis as a result of hyperphosphorylation and degradation of Wee1 (Watanabe et al., 1995, EMBO 14:1878-91; McGowan and Russell, 1993, EMBO 12:75-85). Recent work in Xenopus and fission yeast has demonstrated that Cdk1 (Cdc2) can phosphorylate Wee1, suggesting a positive-feedback loop model in which a small amount of mitotic Cdk1 inactivates Wee1, and subsequently triggers a significant increase in mitotic Cdk1. Tome-1 also promotes mitotic entry by targeting Wee1 for proteolytic destruction by SCF in G2 phase. APC CDH allows Wee1 reinstatement in S phase by destruction of Tome-1 and cyclin B during G1 phase (reviewed by Lim and Surana, 2003, Mol. Cell 11:845-851).

A new role has also been suggested for Wee1 in apoptosis. Crk, which has been implicated in apoptosis in Xenopus, can bind with Wee1 via its SH2 domain. Exogenous Wee1 accelerated Xenopus egg apoptosis in a Crk dependent manner (Smith et al., 2000, J. Cell Biol. 151:1391-1400). These Crk-Wee1 complexes, in the absence of nuclear export factor Crm1 binding, also promoted apoptosis in mammalian cells (Smith et al, 2002, Mol. Cell. Biol. 22:1412-1423). Studies involving the HIV protein R (Vpr) have also involved Wee1 in apoptotic events (Yuan, et al., 2003, J. Virol. 77:2063-2070). Vpr causes G2 arrest which is associated with Cdc2 inactivation, and prolonged G2 arrest leads to apoptosis. Wee1 was depleted in Vpr induced apoptotic HeLa cells and gamma-irradiated apoptotic HeLa cells. Overexpression of Wee1 attenuated Vpr-induced apoptosis, and depletion of Wee1 by siRNA induced apoptotic death. The apparent conflict between Wee1 levels and apoptotic events in these studies, and the mechanisms of apoptosis induction by Wee1 have not been elucidated.

The role of cell cycle inhibitors is important if DNA is damaged. The block in cell division allows time for DNA repair and minimizes the replication and segregation of damaged DNA. The two cell cycle “checkpoints” for genetic integrity are at the G1 phase (before DNA synthesis) and G2 phase (just before mitosis). Loss of these checkpoint controls facilitates the evolution of cells into cancer (reviewed by Hartwell and Kastan, 1994, Science 266:1821-8).

Defective Wee1 expression may abrogate the G2 checkpoint, facilitating tumor cell proliferation. Wee1 has been found to be significantly suppressed in colon carcinoma cells (reviewed by Lee and Yang, 2001, Cell. Mol. Life Sci. 58:1907-1922). Absence of Wee1 expression was also associated with poorer prognosis and higher recurrency of non-small-cell lung cancer (Yoshida et al., 2004, Ann. Onco. 15:252-256).

In contrast, Wee1 levels and kinase activity was also elevated in hepatocellular carcinoma compared to the surrounding cirrhotic tissue (Masaki et al., 2003, Hepatology 37:534-543).

Alternatively, abrogation of the G2 checkpoint may enhance chemotherapy against G1 checkpoint defective tumor cells. Many tumor cells lack a functional p53 gene, and do not demonstrate a G1 checkpoint. While normal cells would arrest at G1 after DNA damage from irradiation or chemotherapy, the cancer cells would rely upon G2 checkpoint for DNA repair. Abrogation of the G2 checkpoint would therefore be more detrimental to cancer cells than normal cells. A chemical library screen for compounds which selectively inhibit Wee1 has been used to search for anti-cancer agents which inhibit G2 checkpoint because of Wee1's negative regulation of Cdc2 and Wee1's attenuation of apoptosis (Wang et al., 2001, Cancer Res. 61:8211-8217). PD0166285 Wee1 kinase inhibitor demonstrated inhibition of Cdc2 phosphorylation, abrogation of G2 arrest, and sensitized killing of p53 mutant cell lines by radiation. In one embodiment, the invention provides a method of treating a cancer using PD166285 in conjunction with a DNA damaging agent.

Wee1 activation may also be involved in the pathology of rheumatoid arthritis. Growth of rheumatoid synovial cells is tumor-like; cells possess abundant cytoplasm, large nuclei, and karyotypic changes. These transformed cells are found in the cartilage and bone of human RA and animal models. Rheumatoid synovial cell growth is disorganized and anchorage-independent. C-Fos/Ap-1 trasncription factor was increased in rheumatoid synovium. Kawasaki et al. (Kawasaki et al., 2003, Onco. 22:6839-6844) demonstrated that Wee1 is transactivated by c-Fos/AP-1; c-Fos and Wee1 was significantly increased in rheumatoid synovial cells compared to osteoarthritis cells. These synovial cells also displayed increased tetraploidy. Inactivating Wee1 may alleviate some of the joint destruction that occurs in RA.

U.S. 20030087847 A1 describes a method for using nucleic acids molecules to inhibit Chk1 activity, as a way to abrogate the G2 checkpoint and selectively sensitive p53 deficient tumors to chemotherapy. Chk1 phosphorylates an inhibitory residue on Cdc25, which is an activator of Cdc2. EP1360281 A2 describes Wee1 nucleotide and amino acid sequences, methods for expression of recombinant Wee1, and identifying compounds that modulate Wee1 activity.

In another preferred embodiment, the invention provides EPHB3 as a gene that is capable of reducing or enhancing cell killing by DNA damaging agents. Receptor tyrosine kinases (RTK) are membrane spanning proteins with an extra-cellular ligand binding domain and intracellular kinase domain. With 14 members, the Eph receptors comprise the largest subfamily of RTK. The extracellular region of The extracellular portion of Eph receptors is composed of a putative immunoglobulin (Ig) region (ligand binding domain), followed by a cysteine-rich region, and two fibronectin type III repeats near the single transmembrane segment (Connor and Pasquale, 1995 Oncogene 11:2429-2438; Labrador et al., 1997, EMBO 16:3889-3897). The cytoplasmic portion contains a highly conserved tyrosine kinase domain flanked by a juxtamembrane region and a C-terminal tail (sterile a motif and PDZ-binding motif), which are less conserved. Eph receptors are divided into two groups based on the sequence homologies of their extracellular domains. The EphA receptors interact with high affinity to ephrin-A ligands, which are tethered to the cell surface by a glycosylphophatidylinositol (GPI) anchor. EphB receptors preferentially bind the transmembrane ephrin-B ligands. With each group, receptors can bind to more than one ligand, and each ligand can bind to more than one receptor. There is less receptor-ligand cross-talk between the A and B subgroups (reviewed in Orioli and Klein, 1997 Trends in Genetics 13:354-359; Pasquale, 1997 Curr. Biol. 9:608-615). Eph receptors can only be activated by membrane-bound or artificially-clustered ephrins; while soluble ligands do bind the receptors, they do not trigger receptor autophosphorylation (Davis et al., 1994 Science 266: 816-819). Eph receptors and ephrins are unique in that they mediate bi-directional signaling. Due to their membrane-bound states, Eph receptors and ephrins are thought to mediated cell-to-cell interactions rather than long-range functions.

Expression of the Eph receptors is distinct, but overlapping, suggesting unique but redundant functions. Expression of Eph receptors is highest in the nervous tissue, but can be found in numerous tissues. Expression is higher in the developing embryo, but is also present in adult tissues. Receptor-ligand interactions often result in cell repulsion, and these repulsive effects have been implicated in axonal guidance, synapse formation, segmental patterning of the nervous system, angiogenesis, and cell migration in development. These receptors may also be involved in neural cells, angiogenesis, and tumorigenesis in adults (reviewed in Dodelet and Pasquale, 2000 Oncogene 19:5614-5619; Zhou, 1998 Pharmacol. Ther. 77:151-181; Pasquale, 1997 Curr. Opin. Cell Biol. 9:608-615). Cellular repulsion or de-adhesion appears to be mediated through interaction between the Eph receptor and numerous signaling molecules such as Nck, Ras-GAP, Src, SHEP1, and SHP2 (Wilkinson, 2001 Neurosci. Rev. 2:155-164).

There are eight EphA receptors (EphA1-8) and six EphB (EphB1-6) receptors, all of which encode a protein of about 1000 amino acids. Eph genes have been identified in a number of species such as chicken, rat, mouse, and human. EphB3, also known as Hek2, Sek4, Mdk5, Cek10, or Tyro 6, can interact with ligands ephrin-B1-3 (Pasquale, 1997, Curr. Opin. Cell Biol. 9:608-615). EphB3 sequences are highly conserved among different species (>95% amino acid homology). The single copy 20.2 kb EphB3 gene is located on human chromosome 3 and has 16 exons. The human protein consists of 998 amino acids (ref. seq. NM004443). High levels of mouse EphB3 transcripts are found throughout embryonic development and in adult brain, intestine, placenta, muscle, heart, and with lesser intensity lung and kidney (Ciossek et al., 1995 Oncogene 11:2085-2095). EphB3 transcripts were found in adult human brain, lung, pancreas, liver, placenta, kidney, skeletal muscle, and heart (Bohme et al, 1993 Oncogene 8:2857-2862).

An EphB3 splice variant has been identified in the chicken, which has a 15 amino acid insertion in the juxtamembrane domain (Sajjadi and Pasquale, 1993 Oncogene 8:1807-1813). In addition to the major 4.8 kb full-length EphB3 transcript, smaller 2.8 kb, 2.3 kb, and 1.9 kb transcripts were found in mouse tissues (Ciossek et al., 1995 Oncogene 11:2085-2095). Only one transcript size has been observed thus far in human EphB3 (Bohme et al., Oncogene 1993 8:2857-2862). However, a human EphB2 splice variant has been identified, suggesting that additional isoforms of other human Eph receptors may be found (Tang et al., 1998 Oncogene 17:521-526).

Considerable characterization of Eph receptors has been done in embryo development. Adams et al. (Genes & Dev. 13:295-306), showed that EphB3 is expressed in the yolk sacs and developing arteries and veins of embryonic mice. They also demonstrated that EphB2/EphB3 double mutant mice display defects in yolk sac vascularization, extended pericardial sacs, defective vascular development, and defective angiogenesis of the head, heart, and somites. Adams et al. also determined that ephrin-B ligands are able to induce capillary sprouting in an in vitro assay.

EphB3 deficient mice implicate the receptor's involvement in the formation of brain commissures, specifically the corpus callosum which connects the two cerebral hemispheres. Furthermore EphB2/EphB3 double mutants have cleft palates, suggesting their involving in facial development as well (Orioli et al., 1996 EMBO 15:6035-6049).

Within the intestinal epithelium, stem cells produce precursors that migrate in specific patterns as they differentiate. Mutational activation of β-catenin/TCF in intestinal epithelial cells results in polyp formation. Batle et al. showed that β-catenin/TCF signaling events control EphB3 expression in colorectal cancer cells and along the crypt-villus axis. In EphB3 null mice, Paneth cells, which normally migrate to occupy the bottom of the intestinal crypts, were randomly localized throughout the crypt, suggesting a deficiency in sorting cell populations. Furthermore, in EphB2/EphB3 double mutants, proliferative and differentiated cells intermingled in the intestinal epithelium (Batle et al., 2002 Cell 111:251-263).

EphB3 expression has also been found in adult mouse cochlea, suggesting a possible role in the peripheral auditory system. EphB3 knockout mice exhibited significantly lower distortion-product otoacoustic emissions DPOAE levels compared to wild type controls (Howard et al., 2003 Hear. Res. 178:118-130). DPOAE measurements reflect cochlear function at the level of outer hair cells.

Willson et al. demonstrated upregulation of EphB3 expression in the injured spinal cords of adult rats, at the injury site (Willson et al., 2003, Cell Transpl. 12:279-290). Expression of EphB3 receptors was co-localized in regions of the CNS which also had a high level of ephrin B ligands. The complementary expression of both EphB3 receptor and ligand at the site of injury may contribute to an environment that inhibits axonal regeneration after injury.

EphB3 has been detected in tumor cell lines of breast and epidermoid origin (Bohme et al., 1993, Oncogene 8:2857-2862). Expression levels of other Eph receptors are upregulated in various tumor types as well (reviewed in Dodelet and Pasquale, Oncogene 2000 19:5614-5619). Some evidence suggests that upregulation of Eph receptors does not appear to drive proliferation (Lhotak and Pawson, 1993, Mol. Cell. Biol. 13:7071-7079), but rather elevated expression appears to correlate with metastatic potential (Andres et al., 1994 Oncogene 1461-1467; Vogt et al., 1998 Clin. Cancer Res. 4:791-797).

Tissue disorganization and abnormal cell adhesion are hallmarks of advanced tumors. Overexpression Eph receptors may make tumors highly sensitive to ephrin activation, promoting decreased cell adhesion, cell motility, and invasiveness. Eph receptors have been found to influence cell-matrix attachment by modulating integrin activity. Maio et al. (2000 Nature Cell. Biol. 2:62-69) has shown that activation of EphA2 with the ephrinA1 ligand on prostate carcinoma cells transiently inhibits integrin-mediated cell attachment. Additionally, in early Xenopus embryos, ectopic expression of ephrin-B1 or activated EphA4 interfered with cadherin dependent cell attachment (Jones et al, 1998 Proc. Natl. Acad. Sci. USA 95:576-581; Winning et al, 1996 Dev. Biol., 179:309-319).

Links between Eph receptors and cytoskeletal changes, a key aspect of cellular motility, have also been established. Activation of EphB4 by ephrin-B2 ligand induces Rac-mediated membrane ruffling in Eph expressing cells (Marston et al., 2003 Nat. Cell Biol. 5:879-888). Wahl et al. (2000 J. Cell Biol. 149:263-270) has demonstrated that ephrin-A5 induces collapse of neural growth cones in a Rho-dependent manner. Both Rho and Rac have been implicated in the cellular changes involved in a tumor formation (reviewed in Schmitz et al., 2000 Exp. Cell Res. 261:1-12). Activation of these signaling pathways by Eph receptors may contribute to tumor invasion and metastasis.

Given the role of Eph receptors and their ligands in embryonic vascular development, and angiogenesis (reviewed in Sullivan and Bicknell, 2003 Br. J. Cancer 89:228-231), these molecules may also be involved in tumor growth by contributing to vascularization of tumors. Eph receptor ligands have been shown to promote organization and assembly of endothelial cells into capillary structures, and to induce capillary sprouting from existing blood vessels (Daniel et al., 1996 Kidney Intl. Suppl. 57:S73-81; Pandey et al., 1995 Science 268:567-569). Secreted ephrin ligands may also act as diffusible chemoattractants for endothelial cells; eph receptors expressed on tumor cells may guide the construction of new vessels from incoming endothelial cells (Pandey et al., 1995 Science 268:567-569).

Because of its upregulation in tumor cells (Bohme et al., 1993 Oncogene 8:2857), and its potential involvement in tumor angiogenesis and metastasis, EphB3 may make an attractive target for cancer diagnosis or therapeutic intervention. Soluble EphA-F_{c} receptors inhibited tumor angiogenesis in cutaneous window assays and in vivo in mice which were injected with 4T1 tumor cells Brantley et al, 2002 Oncogene 21:7011-7026).

Alternatively, there may be situations where enhancement of the angiogenesis properties of Eph receptors may be desirable, such as for treatment for coronary vessel blockage.

The expression of EphB3 in injured spinal cords may also serve as an attractive therapeutic target for CNS injury. The cell repulsive effects of EphB3 may contribute to inability of injured spinal cord axons to regrow. Studies have demonstrated axonal regrowth in the injured spinal cord when other molecules inhibitory for axonal regeneration are blocked by antibodies (Bregman et al., 1995 Nature 378:498-501; GrandPre et al., 2002 Nature 417:547-551).

Eph receptor autophosphorylation is a key event for subsequent interaction with other signaling molecules with SH2 of phosphotyrosine binding domains (reviewed in Bruckner et al, 1998 Curr. Opion. Neuro. 8:375-382).

Binns et al. (Binns, et al., 2000, Mol. Cell. Biol. 20:4791-4805) describes a cellular assay system for studying ephrin-stimulation of EphB2 on neuronal cells. Briefly, an NG108-15 cell line stably expressing EphB2 (NG-EphB2WT cells) was established. NG108-15 cells display characteristics of motor neurons, a cell type which expresses EphB2 during embryonic development. NG108-15 cells, however, do not endogenously express EphB2 or respond to ephrin-B ligands. Stimulation of NG-EphB2WT cells with Fc-ephrin-B1 results in neurite retraction and disassembly of polymerized actin structures. Wildtype NG108-15 cells and cells expressing tyrosine-to-phenylalanine substitutions (key phosphorylation sites) in the juxtamembrane motif do not exhibit the cytoskeletal remodeling in response to ligand stimulation. Variation in phosphorylation of tyrosine residues in wt EphB2 vs. EphB2(Y→F) transformed cells was also monitored with anti-p Tyr antibodies. Decreased EphB2 receptor function also resulted in decreased phosphorylation of p62^{dok}, a component of the eph signaling cascade.

U.S. Pat. No. 6,169,167 also describes methods of determining hek4 activation with Hek4 ligands using a cell-cell autophosphorylation assay. Following receptor-ligand interaction, Hek4 receptors are immunoprecipitated from lysates of CHO cells expressing Hek4 DNA. The lysates are used in Western blots with anti-phosphotyrosine antibodies.

In still another preferred embodiment, the invention provides RAD51 as a gene that is capable of reducing or enhancing cell killing by DNA damaging agents. In mammalian cells, double strand DNA breaks (DSBs) can be repaired by non-homologous end joining (NHEJ) or by homologous recombination. NHEJ involves the re-ligation of broken DNA ends without a template and may result in mutations or deletions at the break site. Homologous recombination requires a template, an intact sister duplex, and results in high fidelity repair. Homologous recombination can also repair stalled or broken replication forks in DNA. Repair of DSBs is vital as impaired function or apoptosis may occur if they are left undone or repaired inaccurately. Genetic instability, a key characteristic of tumor cells, may also result without the high fidelity of homologous recombinational repair. The initial steps of homologous recombination, homologous pairing and strand exchange, involve a protein belonging to the RecA/Rad51 recombinase family (reviewed in Baumann and West, 1998, Trends Biochem. Sci. 23:247-251; Henning and Stürzbecher, 2003, Toxicology 193:91-109).

The E. coli protein RecA acts as a regulator of the SOS response to DNA damage and promotes homologous pairing and strand exchange (reviewed in Baumann and West, 1998, Trends Biochem. Sci. 23:247-251). A DSB repair gene rad51 was identified in Saccharomyces cerevisiae and is homologous to recA (Shinohara et al., 1992, Cell 69:457-470). The rad51 gene was also cloned from human and mouse (Yoshimura et al., 1993, Nucleic Acids Res. 21:1665; Shinohara et al., 1993, Nature Genet. 4:239-243). The single copy human rad51 gene is located on chromosome 15 (Shinohara et al, 1993, Nature Genet. 4:239-243). The rad51 gene consists of 10 exons, encoding a 339 amino acid protein. The amino acid sequence of the two mammalian Rad51 proteins is 83% homologous to the yeast Rad51, and 56% homologous to the E. coli RecA protein. The regions of homology between RecA and Rad51 include functional domains for recombination, UV resistance, and oligomer formation (positions 31-260 of RecA) (Yoshimura et al., 1993, Nucleic Acids Res. 21:1665; Shinohara et al., 1993, Nature Genet. 4:239-243). Mouse Rad51 transcripts were found at high levels in thymus, spleen, testis, and ovary, and at lower levels in the brain (Shinohara et al, 1993, Nature Genet. 4:239-243). Rad51 expression also appears to be cell cycle regulated, with transcriptional upregulation at S and G2 phases (Flygare et al., 1996, Biochim. Biophys. Acta 1312:231-236). Additionally, five Rad51 paralogs have been identified (XRCC2, XRCC3, Rad51B-D) that have 20-30% identity with Rad51. These paralogs may promote Rad51 focus formation (reviewed in Thompson and Schild, 2001, Mutat. Res. 477:131-153).

Rad51 functions as a long helical polymer that wraps around DNA to form a nucleoprotein filament. Rad51 binds to single stranded DNA produced by nucleolytic resection at the DSB site, and this interaction is enhanced by Rad52. Invasion of a re-sected end of the DSB into a homologous duplex occurs in the Rad51 nucleoprotein filament, requiring ATP-binding but not hydrolysis. The second re-sected end is also captured by Rad51. The invading re-sected ends function as primers for DNA re-synthesis. Holliday-junction resolution and ligation allow the repaired duplexes to separate (reviewed by West, 2003, Nat. Rev. Mol. Cell. Biol. 4:435-445). Pellegrini et al. (2002, Nature 420:287-293) reported that a conserved repeat sequence in BRCA2, BRC4, mimics a motif in Rad51 and serves as an interface for oligomerization of Rad51 monomers. Through this BRC4-Rad51-complex, BRCA2 is able to control the assembly of the Rad51 nucleoprotein filament. Rad51 activity is also regulated by other mechanisms. P53 has been found to down-modulate homologous recombination promoted by Rad51 (Linke et al., 2003, Cancer Res. 63:2596-2605; Yoon et al., 2004, J. Mol. Biol. 336:639-654). Rad54 has been found to disassemble Rad51 nucleoprotein filaments formed on double stranded DNA (dsDNA) and may be involved in turnover of Rad51-dsDNA filaments, which is important during DNA strand exchange reactions. In yeast, Srs2 has been found to inhibit recombination by disrupting Rad51 filament formation on single stranded DNA (Veaute et al., 2003, Nature 423:309-312; Krejci et al., 2003, Nature 423:305-309).

Splice variants of Rad51 have been identified. One transcript (NM_{—}133487) lacks an internal segment corresponding to exons 4, 5 and the 5′ portion of exon 6, resulting in a protein that lacks an internal region of 97 amino acids. The transcript identified by the Genbank accession number AY425955 also suggests the existence of a further truncated splice variant in testis. Rad51 splice variants have also been found in other species, such as C. elegans (Rinaldo et al., 1998, Mol. Gen. Genet. 260:289-294).

A couple of studies have demonstrated that a Rad51 135C polymorphism significantly elevates the risk of breast cancer in carriers of BRCA2 but not BRCA1 (Levy-Lahad et al., 2001, Proc. Natl. Acad. Sci. USA 98:3232-3236; Kadouri et al., 2004, Br. J. Cancer 90:2002-2005). A missense mutation (Gln150Arg) was reported in two patients with bilateral breast cancer, but otherwise, Rad51 mutations were not found in most tumors (Kato et al., 2000, J. Hum. Genet. 45:133-137; Schmutte et al., 1999, Cancer Res. 59:4564-4569). Rad51 knockout mice die early during embryonic development, though heterozygotes are viable and fertile, and rad51^{-/-} mouse cell lines could not be established, indicating an essential role for this gene (Tsuzuki et al., 1996, Proc. Natl. Acad. Sci. USA 93:6236-6240). Sonoda et al. (1998, EMBO J., 17:598-608) generated a rad51^{-/-} chicken B lymphocyte DT40 cell line by using a Rad51 transgene controlled by a repressible promoter. Inhibition of the rad51 transgene in DT40 cells resulted in high levels of chromosome breakage, cell cycle arrest at the G₂/M phase, and cell death. Several studies have also investigated Rad51 overexpression in cell lines. Vispe et al. (1998, Nucleic Acids Res. 26:2859-2864) found that Rad51 overexpression in CHO cells resulted in a 20-fold increase in homologous recombination between two adjacent homologous alleles and increased resistance to ionizing radiation in the late S/G₂ cell cycle phase. Work done by Richardson et al. (2004, Oncogene 23:546-553) presents evidence for a link between increased levels of Rad51 in tumor cells and chromosomal instability associated with tumor progression. Rad51 levels transiently upregulated 2-4-fold during induction of DSB in a mouse ES cell line produced novel recombinational repair products and generation of abnormal karyotypes.

Elevated Rad51 levels have been reported in tumors, suggesting that Rad51 up-regulation may confer an advantage to tumor progression. Maacke et al. (2000, Int. J. Cancer 88:907-913) reported a positive correlation between Rad51 overexpression and breast tumor grading. A 2-7-fold increase of Rad51 was also observed in a wide range of tumor cell lines compared to nonmalignant control cell lines (Raderschall et al., 2002, Cancer Res. 62:219-225). Rad51 overexpression was also found in 66% of human pancreatic adenocarcinoma tissue samples (Maacke et al., 2000, Oncogene 19:2791-2795). It is speculated that Rad51 overexpression in cancer cells may protect cells from DNA damage or contribute to genomic instability and diversity. Elevated expression of Rad51 and increased recombination was also observed during immortalization of human fibroblasts (Xia et al., 1997, Mol. Cell Biol. 17:7151-7158).

A number of studies have suggested a functional role for Rad51 in tumor resistance. Hansen et al. (2003, Int. J. Cancer 105:472-479) demonstrated that Rad51 levels positively correlated with etoposide resistance in small cell lung cancer (SCLC) cells. Furthermore, down or upregulation of Rad51 using sense or antisense constructs altered etoposide sensitivity in SCLC cells. Chlorambucil treatment was found to induce Rad51 expression in B-cell chronic lymphocytic leukemia cells (Christodoulopoulos et al., 1999, Clin. Cancer Res. 5:2178-2184). Antisense Rad51 oligonucleotides enhanced DNA damage by irradiation in both a mouse embryonic skin cell line and malignant gliomas (Taki et al., 1996, Biochem. Biophys. Res. Commun. 223:434-438; Ohnishi et al., 1998, Biochem. Biophys. Res. Commun. 245:319-324). Downregulation of Rad51 with ribozymes also increased the sensitivity of prostate cancer cells to irradiation (Collis et al., 2001, Nucleic Acids Res. 29:1534-1538). Disruption of Rad51 function through its interaction with BRC repeats on BRCA2 also leads to radiation and methyl methanesulfonate hypersensitivity in cancer cells (Chen et al., 1999, J. Biol. Chem. 274:32931-32935; Chen et al., 1998, Proc. Natl. Acad. Sci. USA 95:5287-5292). Slupianek et al. (2001, Mol. Cell 8:795-806) showed that Bcr/Abl regulation of Rad51 expression is important for cisplatin and mitomycin C resistance in myeloid cells. These studies suggest Rad51 as an attractive target to improve the efficacy of cancer therapy.

### 5.4.2. DNA Damaging Agents

The invention can be practiced with any known DNA damaging agent, including but are not limited to any topoisomerase inhibitor, DNA binding agent, anti-metabolite, ionizing radiation, or a combination of two or more of such known DNA damaging agents.

A topoisomerase inhibitor that can be used in conjunction with the invention can be a topoisomerase I (Topo I) inhibitor, a topoisomerase II (Topo II) inhibitor, or a dual topoisomerase I and II inhibitor. A topo I inhibitor can be from any of the following classes of compounds: camptothecin analogue (e.g., karenitecin, aminocamptothecin, lurtotecan, topotecan, irinotecan, BAY 56-3722, rubitecan, G114721, exatecan mesylate), rebeccamycin analogue, PNU 166148, rebeccamycin, TAS-103, camptothecin (e.g., camptothecin polyglutamate, camptothecin sodium), intoplicine, ecteinascidin 743, J-107088, pibenzimol. Examples of preferred topo I inhibitors include but are not limited to camptothecin, topotecan (hycaptamine), irinotecan (irinotecan hydrochloride), belotecan, or an analogue or derivative thereof.

A topo II inhibitor that can be used in conjunction with the invention can be from any of the following classes of compounds: anthracycline antibiotics (e.g., carubicin, pirarubicin, daunorubicin citrate liposomal, daunomycin, 4-iodo-4-doxydoxorubicin, doxorubicin, n,n-dibenzyl daunomycin, morpholinodoxorubicin, aclacinomycin antibiotics, duborimycin, menogaril, nogalamycin, zorubicin, epirubicin, marcellomycin, detorubicin, annamycin, 7-cyanoquinocarcinol, deoxydoxorubicin, idarubicin, GPX-100, MEN-10755, valrubicin, KRN5500), epipodophyllotoxin compound (e.g., podophyllin, teniposide, etoposide, GL331, 2-ethylhydrazide), anthraquinone compound (e.g., ametantrone, bisantrene, mitoxantrone, anthraquinone), ciprofloxacin, acridine carboxamide, amonafide, anthrapyrazole antibiotics (e.g., teloxantrone, sedoxantrone trihydrochloride, piroxantrone, anthrapyrazole, losoxantrone), TAS-103, fostriecin, razoxane, XK469R, XK469, chloroquinoxaline sulfonamide, merbarone, intoplicine, elsamitrucin, CI-921, pyrazoloacridine, elliptinium, amsacrine. Examples of preferred topo II inhibitors include but are not limited to doxorubicin (Adriamycin), etoposide phosphate (etopofos), teniposide, sobuzoxane, or an analogue or derivative thereof.

DNA binding agents that can be used in conjunction with the invention include but are not limited to DNA groove binding agent, e.g., DNA minor groove binding agent; DNA crosslinking agent; intercalating agent; and DNA adduct forming agent. A DNA minor groove binding agent can be an anthracycline antibiotic, mitomycin antibiotic (e.g., porfiromycin, KW-2149, mitomycin B, mitomycin A, mitomycin C), chromomycin A3, carzelesin, actinomycin antibiotic (e.g., cactinomycin, dactinomycin, actinomycin F1), brostallicin, echinomycin, bizelesin, duocarmycin antibiotic (e.g., KW 2189), adozelesin, olivomycin antibiotic, plicamycin, zinostatin, distamycin, MS-247, ecteinascidin 743, amsacrine, anthramycin, and pibenzimol, or an analogue or derivative thereof.

DNA crosslinking agents include but are not limited to antineoplastic alkylating agent, methoxsalen, mitomycin antibiotic, psoralen. An antineoplastic alkylating agent can be a nitrosourea compound (e.g., cystemustine, tauromustine, semustine, PCNU, streptozocin, SarCNU, CGP-6809, carmustine, fotemustine, methylnitrosourea, nimustine, ranimustine, ethylnitrosourea, lomustine, chlorozotocin), mustard agent (e.g., nitrogen mustard compound, such as spiromustine, trofosfamide, chlorambucil, estramustine, 2,2,2-trichlorotriethylamine, prednimustine, novembichin, phenamet, glufosfamide, peptichemio, ifosfamide, defosfamide, nitrogen mustard, phenesterin, mannomustine, cyclophosphamide, melphalan, perfosfamide, mechlorethamine oxide hydrochloride, uracil mustard, bestrabucil, DHEA mustard, tallimustine, mafosfamide, aniline mustard, chlomaphazine; sulfur mustard compound, such as bischloroethylsulfide; mustard prodrug, such as TLK286 and ZD2767), ethylenimine compound (e.g., mitomycin antibiotic, ethylenimine, uredepa, thiotepa, diaziquone, hexamethylene bisacetamide, pentamethylmelamine, altretamine, carzinophilin, triaziquone, meturedepa, benzodepa, carboquone), alkylsulfonate compound (e.g., dimethylbusulfan, Yoshi-864, improsulfan, piposulfan, treosulfan, busulfan, hepsulfam), epoxide compound (e.g., anaxirone, mitolactol, dianhydrogalactitol, teroxirone), miscellaneous alkylating agent (e.g., ipomeanol, carzelesin, methylene dimethane sulfonate, mitobronitol, bizelesin, adozelesin, piperazinedione, VNP40101M, asaley, 6-hydroxymethylacylfulvene, EO9, etoglucid, ecteinascidin 743, pipobroman), platinum compound (e.g., ZD0473, liposomal-cisplatin analogue, satraplatin, BBR 3464, spiroplatin, ormaplatin, cisplatin, oxaliplatin, carboplatin, lobaplatin, zeniplatin, iproplatin), triazene compound (e.g., imidazole mustard, CB10-277, mitozolomide, temozolomide, procarbazine, dacarbazine), picoline compound (e.g., penclomedine), or an analogue or derivative thereof. Examples of preferred alkylating agents include but are not limited to cisplatin, dibromodulcitol, fotemustine, ifosfamide (ifosfamid), ranimustine (ranomustine), nedaplatin (latoplatin), bendamustine (bendamustine hydrochloride), eptaplatin, temozolomide (methazolastone), carboplatin, altretamine (hexamethylmelamine), prednimustine, oxaliplatin (oxalaplatinum), carmustine, thiotepa, leusulfon (busulfan), lobaplatin, cyclophosphamide, bisulfan, melphalan, and chlorambucil, or analogues or derivatives thereof.

Intercalating agents can be an anthraquinone compound, bleomycin antibiotic, rebeccamycin analogue, acridine, acridine carboxamide, amonafide, rebeccamycin, anthrapyrazole antibiotic, echinomycin, psoralen, LU 79553, BW A773U, crisnatol mesylate, benzo(a)pyrene-7,8-diol-9,10-epoxide, acodazole, elliptinium, pixantrone, or an analogue or derivative thereof.

DNA adduct forming agents include but are not limited to enediyne antitumor antibiotic (e.g., dynemicin A, esperamicin A1, zinostatin, dynemicin, calicheamicin gamma 1I), platinum compound, carmustine, tamoxifen (e.g., 4-hydroxy-tamoxifen), psoralen, pyrazine diazohydroxide, benzo(a)pyrene-7,8-diol-9,10-epoxide, or an analogue or derivative thereof.

Anti-metabolites include but are not limited to cytosine, arabinoside, floxuridine, fluorouracil, mercaptopurine, Gemcitabine, and methotrexate (MTX).

Ionizing radiation includes but is not limited to x-rays, gamma rays, and electron beams.

### 5.4.3. Methods of Determining Proteins or Other Molecules that Interact with a DNA Damage Response Gene

Any method suitable for detecting protein-protein interactions may be employed for identifying interaction of DNA damage response protein with another cellular protein. The interaction between DNA damage response gene and other cellular molecules, e.g., interaction between DNA damage response and its regulators, may also be determined using methods known in the art.

Among the traditional methods which may be employed are co-immunoprecipitation, crosslinking and co-purification through gradients or chromatographic columns. Utilizing procedures such as these allows for the identification of cellular proteins which interact with DNA damage response gene products. Once isolated, such a cellular protein can be identified and can, in turn, be used, in conjunction with standard techniques, to identify proteins it interacts with. For example, at least a portion of the amino acid sequence of the cellular protein which interacts with the DNA damage response gene product can be ascertained using techniques well known to those of skill in the art, such as via the Edman degradation technique (see, e.g., Creighton, 1983, “Proteins: Structures and Molecular Principles”, W.H. Freeman & Co., N.Y., pp. 34-49). The amino acid sequence obtained may be used as a guide for the generation of oligonucleotide mixtures that can be used to screen for gene sequences encoding such cellular proteins. Screening may be accomplished, for example, by standard hybridization or PCR techniques. Techniques for the generation of oligonucleotide mixtures and the screening are well-known. (See, e.g., Ausubel, supra., and PCR Protocols: A Guide to Methods and Applications, 1990, Innis, M. et al., eds. Academic Press, Inc., New York).

Additionally, methods may be employed which result in the simultaneous identification of genes which encode the cellular protein interacting with the DNA damage response protein. These methods include, for example, probing expression libraries with labeled DNA damage response protein, using DNA damage response protein in a manner similar to the well known technique of antibody probing of λgt11 libraries.

One method which detects protein interactions in vivo, the two-hybrid system, is described in detail for illustration only and not by way of limitation. One version of this system has been described (Chien et al., 1991, Proc. Natl. Acad. Sci. USA, 88:9578-9582) and is commercially available from Clontech (Palo Alto, Calif.).

Briefly, utilizing such a system, plasmids are constructed that encode two hybrid proteins: one consists of the DNA-binding domain of a transcription activator protein fused to the DNA damage response gene product and the other consists of the transcription activator protein's activation domain fused to an unknown protein that is encoded by a cDNA which has been recombined into this plasmid as part of a cDNA library. The DNA-binding domain fusion plasmid and the cDNA library are transformed into a strain of the yeast Saccharomyces cerevisiae that contains a reporter gene (e.g., HBS or lacZ) whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology may be used to screen activation domain libraries for proteins that interact with the “bait” gene product. By way of example, and not by way of limitation, DNA damage response gene products may be used as the bait gene product. Total genomic or cDNA sequences are fused to the DNA encoding an activation domain. This library and a plasmid encoding a hybrid of a bait DNA damage response gene product fused to the DNA-binding domain are cotransformed into a yeast reporter strain, and the resulting transformants are screened for those that express the reporter gene. For example, and not by way of limitation, a bait DNA damage response gene sequence, such as the coding sequence of a DNA damage response gene can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait DNA damage response gene product are to be detected can be made using methods routinely practiced in the art. According to the particular system described herein, for example, the cDNA fragments can be inserted into a vector such that they are translationally fused to the transcriptional activation domain of GALA. This library can be co-transformed along with the bait DNA damage response gene-GAL4 fusion plasmid into a yeast strain which contains a lacZ gene driven by a promoter which contains GAL4 activation sequence. A cDNA encoded protein, fused to GAL4 transcriptional activation domain, that interacts with bait DNA damage response gene product will reconstitute an active GAL4 protein and thereby drive expression of the HIS3 gene. Colonies which express HIS3 can be detected by their growth on petri dishes containing semi-solid agar based media lacking histidine. The cDNA can then be purified from these strains, and used to produce and isolate the bait DNA damage response gene-interacting protein using techniques routinely practiced in the art.

The interaction between a DNA damage response gene and its regulators may be determined by a standard method known in the art.

### 5.4.4. Methods of Screening for Agents

The invention provides methods for screening for agents that regulate DNA damage response expression or modulate interaction of DNA damage response with other proteins or molecules.

### 5.4.4.1. Screening Assays

The following assays are designed to identify compounds that bind to DNA damage response gene or gene products, bind to other cellular proteins that interact with a DNA damage response gene product, bind to cellular constituents, e.g., proteins, that are affected by a DNA damage response gene product, or bind to compounds that interfere with the interaction of the DNA damage response gene or gene product with other cellular proteins and to compounds which modulate the activity of DNA damage response gene (i.e., modulate the level of DNA damage response gene expression and/or modulate the level of DNA damage response gene product activity). Assays may additionally be utilized which identify compounds which bind to DNA damage response gene regulatory sequences (e.g., promoter sequences), see e.g., Platt, K. A., 1994, J. Biol. Chem. 269:28558-28562, which is incorporated herein by reference in its entirety, which may modulate the level of DNA damage response gene expression. Compounds may include, but are not limited to, small organic molecules which are able to affect expression of the DNA damage response gene or some other gene involved in the DNA damage response pathways, or other cellular proteins. Methods for the identification of such cellular proteins are described, above, in Section 5.4.3. Such cellular proteins may be involved in the regulation of the growth inhibitory effect of a DNA damaging agent. Further, among these compounds are compounds which affect the level of DNA damage response gene expression and/or DNA damage response gene product activity and which can be used in the regulation of resistance to the growth inhibitory effect of a DNA damaging agent.

Compounds may include, but are not limited to, peptides such as, for example, soluble peptides, including but not limited to, Ig-tailed fusion peptides, and members of random peptide libraries; (see, e.g., Lam, K. S. et al., 1991, Nature 354:82-84; Houghten, R. et al., 1991, Nature 354:84-86), and combinatorial chemistry-derived molecular library made of D- and/or L-configuration amino acids, phosphopeptides (including, but not limited to members of random or partially degenerate, directed phosphopeptide libraries; see, e.g., Songyang, Z. et al., 1993, Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope-binding fragments thereof), and small organic or inorganic molecules.

Compounds identified via assays such as those described herein may be useful, for example, in regulating the biological function of the DNA damage response gene product, and for ameliorating resistance to the growth inhibitory effect of a DNA damaging agent and/or enhancing the growth inhibitory effect of a DNA damaging agent. Assays for testing the effectiveness of compounds are discussed, below, in Section 5.4.4.2.

In vitro systems may be designed to identify compounds capable of binding the DNA damage response gene products of the invention. Compounds identified may be useful, for example, in modulating the activity of wild type and/or mutant DNA damage response gene products, may be useful in elaborating the biological function of the DNA damage response gene product, may be utilized in screens for identifying compounds that disrupt normal DNA damage response gene product interactions, or may in themselves disrupt such interactions.

The principle of the assays used to identify compounds that bind to the DNA damage response gene product involves preparing a reaction mixture of the DNA damage response gene product and the test compound under conditions and for a time sufficient to allow the two components to interact and bind, thus forming a complex which can be removed and/or detected in the reaction mixture. These assays can be conducted in a variety of ways. For example, one method to conduct such an assay would involve anchoring DNA damage response gene product or the test substance onto a solid phase and detecting DNA damage response gene product/test compound complexes anchored on the solid phase at the end of the reaction. In one embodiment of such a method, the DNA damage response gene product may be anchored onto a solid surface, and the test compound, which is not anchored, may be labeled, either directly or indirectly.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for DNA damage response gene product or the test compound to anchor any complexes formed in solution, and a labeled antibody specific for the other component of the possible complex to detect anchored complexes.

The DNA damage response gene or gene products may interact in vivo with one or more intracellular or extracellular molecules, such as proteins. Such molecules may include, but are not limited to, nucleic acid molecules and those proteins identified via methods such as those described, above, in Section 5.4.3. For purposes of this discussion, such molecules are referred to herein as “binding partners”. Compounds that disrupt DNA damage response gene product binding may be useful in regulating the activity of the DNA damage response gene product. Compounds that disrupt DNA damage response gene binding may be useful in regulating the expression of the DNA damage response gene, such as by regulating the binding of a regulator of DNA damage response gene. Such compounds may include, but are not limited to molecules such as peptides, and the like, as described, for example, in Section 5.4.4.1. above, which would be capable of gaining access to the DNA damage response gene product.

The basic principle of the assay systems used to identify compounds that interfere with the interaction between the DNA damage response gene product and its intracellular or extracellular binding partner or partners involves preparing a reaction mixture containing the DNA damage response gene product, and the binding partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of DNA damage response gene product and its binding partner. Control reaction mixtures are incubated without the test compound or with a placebo. The formation of any complexes between the DNA damage response gene protein and the binding partner is then detected. The formation of a complex in the control reaction, but not in the reaction mixture containing the test compound, indicates that the compound interferes with the interaction of the DNA damage response gene protein and the interactive binding partner. Additionally, complex formation within reaction mixtures containing the test compound and normal DNA damage response gene protein may also be compared to complex formation within reaction mixtures containing the test compound and a mutant DNA damage response gene protein. This comparison may be important in those cases wherein it is desirable to identify compounds that disrupt interactions of mutant but not normal DNA damage response gene proteins.

The assay for compounds that interfere with the interaction of the DNA damage response gene products and binding partners can be conducted in a heterogeneous or homogeneous format. Heterogeneous assays involve anchoring either the DNA damage response gene product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the DNA damage response gene products and the binding partners, e.g., by competition, can be identified by conducting the reaction in the presence of the test substance; i.e., by adding the test substance to the reaction mixture prior to or simultaneously with the DNA damage response gene protein and interactive binding partner. Alternatively, test compounds that disrupt preformed complexes, e.g. compounds with higher binding constants that displace one of the components from the complex, can be tested by adding the test compound to the reaction mixture after complexes have been formed. The various formats are described briefly below.

In a heterogeneous assay system, either the DNA damage response gene product or the interactive binding partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the DNA damage response gene product or binding partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds which inhibit complex or which disrupt preformed complexes can be identified.

In an alternate embodiment of the invention, a homogeneous assay can be used. In this approach, a preformed complex of the DNA damage response gene protein and the interactive binding partner is prepared in which either the DNA damage response gene product or its binding partners is labeled, but the signal generated by the label is quenched due to complex formation (see, e.g., U.S. Pat. No. 4,109,496 by Rubenstein which utilizes this approach for immunoassays). The addition of a test substance that competes with and displaces one of the species from the preformed complex will result in the generation of a signal above background. In this way, test substances which disrupt DNA damage response gene protein/binding partner interaction can be identified.

In a particular embodiment, the DNA damage response gene product can be prepared for immobilization using recombinant DNA techniques. For example, the DNA damage response coding region can be fused to a glutathione-5-transferase (GST) gene using a fusion vector, such as pGEX-5X-1, in such a manner that its binding activity is maintained in the resulting fusion protein. The interactive binding partner can be purified and used to raise a monoclonal antibody, using methods routinely practiced in the art. This antibody can be labeled with the radioactive isotope ¹²⁵I, for example, by methods routinely practiced in the art. In a heterogeneous assay, e.g., the GST-DNA damage response fusion protein can be anchored to glutathione-agarose beads. The interactive binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between the DNA damage response gene protein and the interactive binding partner can be detected by measuring the amount of radioactivity that remains associated with the glutathione-agarose beads. A successful inhibition of the interaction by the test compound will result in a decrease in measured radioactivity.

Alternatively, the GST-DNA damage response gene fusion protein and the interactive binding partner can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the DNA damage response gene product/binding partner interaction can be detected by adding the labeled antibody and measuring the radioactivity associated with the beads.

In another embodiment of the invention, these same techniques can be employed using peptide fragments that correspond to the binding domains of the DNA damage response protein and/or the interactive binding partner (in cases where the binding partner is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described in this Section above, and allowed to interact with and bind to its labeled binding partner, which has been treated with a proteolytic enzyme, such as trypsin. After washing, a short, labeled peptide comprising the binding domain may remain associated with the solid material, which can be isolated and identified by amino acid sequencing. Also, once the gene coding for the binding partner is obtained, short gene segments can be engineered to express peptide fragments of the protein, which can then be tested for binding activity and purified or synthesized.

For example, and not by way of limitation, a DNA damage response gene product can be anchored to a solid material as described, above, in this Section by making a GST-DNA damage response fusion protein and allowing it to bind to glutathione agarose beads. The interactive binding partner can be labeled with a radioactive isotope, such as ³⁵S, and cleaved with a proteolytic enzyme such as trypsin. Cleavage products can then be added to the anchored GST-DNA damage response fusion protein and allowed to bind. After washing away unbound peptides, labeled bound material, representing the binding partner binding domain, can be eluted, purified, and analyzed for amino acid sequence by well-known methods. Peptides so identified can be produced synthetically or fused to appropriate facilitative proteins using recombinant DNA technology.

### 5.4.4.2. Screening Compounds that Regulate and/or Enhance the Growth Inhibitory Effect of a DNA Damaging Agent

Any agents that regulate the expression of DNA damage response gene and/or the interaction of DNA damage response protein with its binding partners, e.g., compounds that are identified in Section 5.4.4.1., antibodies to DNA damage response protein, and so on, can be further screened for its ability to regulate and/or enhance the growth inhibitory effect of a DNA damaging agent in cells. Any suitable proliferation or growth inhibition assays known in the art can be used for this purpose. In one embodiment, a candidate agent and a DNA damaging agent are applied to a cells of a cell line, and a change in growth inhibitory effect is determined. Preferably, changes in growth inhibitory effect are determined using different concentrations of the candidate agent in conjunction with different concentrations of the DNA damaging agent such that one or more combinations of concentrations of the candidate agent and DNA damaging agent which cause 50% inhibition, i.e., the IC₅₀, are determined.

In a preferred embodiment, an MTT proliferation assay (see, e.g., van de Loosdrechet, et al., 1994, J. Immunol. Methods 174: 311-320; Ohno et al., 1991, J. Immunol. Methods 145:199-203; Ferrari et al., 1990, J. Immunol. Methods 131: 165-172; Alley et al., 1988, Cancer Res. 48: 589-601; Carmichael et al., 1987, Cancer Res. 47:936-942; Gerlier et al., 1986, J. Immunol. Methods 65:55-63; Mosmann, 1983, J. Immunological Methods 65:55-63) is used to screen for a candidate agent that can be used in conjunction with a DNA damaging agent to inhibit the growth of cells. The cells are treated with chosen concentrations of the candidate agent and a DNA damaging agent for 4 to 72 hours. The cells are then incubated with a suitable amount of 3-(4,5-dimethylthiazol-2-yl)-2,5-diphenyltetrazolium bromide (MTT) for 1-8 hours such that viable cells convert MTT into an intracellular deposit of insoluble formazan. After removing the excess MTT contained in the supernatant, a suitable MTT solvent, e.g., a DMSO solution, is added to dissolved the formazan. The concentration of MTT, which is proportional to the number of viable cells, is then measured by determining the optical density at 570 nm. A plurality of different concentrations of the candidate agent can be assayed to allow the determination of the concentrations of the candidate agent and the DNA damaging agent which causes 50% inhibition.

In another preferred embodiment, an alamarBlue™ Assay for cell proliferation is used to screen for a candidate agent that can be used in conjunction with a DNA damaging agent to inhibit the growth of cells (see, e.g., Page et al., 1993, Int. J. Oncol. 3:473-476). An alamarBlue™ assay measures cellular respiration and uses it as a measure of the number of living cells. The internal environment of proliferating cells is more reduced than that of non-proliferating cells. For example, the ratios of NADPH/NADP, FADH/FAD, FMNH/FMN, and NADH/NAF increase during proliferation. AlamarBlue can be reduced by these metabolic intermediates and, therefore, can be used to monitor cell proliferation. The cell number of a treated sample as measured by alamarBlue can be expressed in percent relative to that of an untreated control sample.

In a specific embodiment, the alamarBlue™ assay is performed to determine whether transfection titration curves of siRNAs targeting DNA damage response genes were changed by the presence of a DNA damaging agent of a chosen concentration, e.g., 6-200 nM of camptothecin. Cells were transfected with an siRNA targeting a DNA damage response gene. 4 hours after siRNA transfection, 100 microliter/well of DMEM/10% fetal bovine serum with or without the DNA damaging agent was added and the plates were incubated at 37° C. and 5% CO₂ for 68 hours. The medium was removed from the wells and replaced with 100 microliter/well DMEM/10% Fetal Bovine Serum (Invitrogen) containing 10% (vol/vol) alamarBlue™ reagent (Biosource International Inc., Camarillo, Calif.) and 0.001 volumes of 1M Hepes buffer tissue culture reagent (Invitrogen). The plates were incubated for 2 hours at 37° C. before they were read at 570 and 600 nm wavelengths on a SpectraMax plus plate reader (Molecular Devices, Sunnyvale, Calif.) using Softmax Pro 3.1.2 software (Molecular Devices). The percent reduced for wells transfected with a titration of an siRNA targeting a DNA damage response gene with or without a DNA damaging agent were compared to luciferase siRNA-transfected wells. The number calculated for % Reduced for 0 nM luciferase siRNA-transfected wells without the DNA damaging agent was considered to be 100%.

### 5.4.4.3. Compounds Identified

The compounds identified in the screen include compounds that demonstrate the ability to selectively modulate the expression of DNA damage response and regulate and/or enhance the growth inhibitory effect of a DNA damaging agent in cells. These compounds include but are not limited to siRNA, antisense, ribozyme, triple helix, antibody, and polypeptide molecules, aptamrs, and small organic or inorganic molecules.

The compounds identified in the screen also include compounds that modulate interaction of DNA damage response with other proteins or molecules. In one embodiment, the compounds identified in the screen are compounds that modulate the interaction of a DNA damage response protein with its interaction partner. In another embodiment, the compounds identified in the screen are compounds that modulate the interaction of DNA damage response gene with a transcription regulator.

### 5.4.5. Diagnostics

A variety of methods can be employed for the diagnostic and prognostic evaluation of cell or cells for their resistance to the growth inhibitory effect of a DNA damaging agent, e.g., camptothecin, cisplatin or doxorubicin, resulting from defective regulation of DNA damage response, and for the identification of subjects having a predisposition to resistance to the growth inhibitory effect of a DNA damaging agent.

In one embodiment, the method comprises determining an expression level of a DNA damage response gene in the cell, in which an expression level above a predetermined threshold level indicates that the cell is DNA damaging agent resistant. Preferably, the predetermined threshold level is at least 2-fold, 4-fold, 8-fold, or 10-fold of the normal expression level of the DNA damage response gene. In another embodiment, the invention provides a method for evaluating DNA damaging agent resistance in a cell comprising determining a level of abundance of a protein encoded by a DNA damage response gene in the cell, in which a level of abundance of the protein above a predetermined threshold level indicates that the cell is DNA damaging agent resistant. In still another embodiment, the invention provides a method for evaluating DNA damaging agent resistance in a cell comprising determining a level of activity of a protein encoded by the DNA damage response gene in cells of the mammal, in which an activity level above a predetermined threshold level indicates that the cell is DNA damaging agent resistant. Preferably, the predetermined threshold level of abundance or activity is at least 2-fold, 4-fold, 8-fold, or 10-fold of the normal level of abundance or activity of the DNA damage response protein.

Such methods may, for example, utilize reagents such as the DNA damage response gene nucleotide sequences and antibodies directed against DNA damage response gene products, including peptide fragments thereof. Specifically, such reagents may be used, for example, for: (1) the detection of the presence of DNA damage response gene mutations, or the detection of either over- or under-expression of DNA damage response gene mRNA relative to the normal expression level; and (2) the detection of either an over- or an under-abundance of DNA damage response gene product relative to the normal DNA damage response protein level.

The methods described herein may be performed, for example, by utilizing pre-packaged diagnostic kits comprising at least one specific DNA damage response gene nucleic acid or anti-DNA damage response antibody reagent described herein, which may be conveniently used, e.g., in clinical settings, to diagnose patients exhibiting DNA damage response related disorder or abnormalities.

For the detection of DNA damage response mutations, any nucleated cell can be used as a starting source for genomic nucleic acid. For the detection of DNA damage response gene expression or DNA damage response gene products, any cell type or tissue in which the DNA damage response gene is expressed may be utilized.

Nucleic acid-based detection techniques are described, below, in Section 5.4.5.1. Peptide detection techniques are described, below, in Section 5.4.5.2.

### 5.4.5.1. Detection of Expression of a DNA Damage Response Gene

The expression of DNA damage response gene in cells or tissues, e.g., the cellular level of DNA damage response transcripts and/or the presence or absence of mutations, can be detected by utilizing a number of techniques. Nucleic acid from any nucleated cell can be used as the starting point for such assay techniques, and may be isolated according to standard nucleic acid preparation procedures which are well known to those of skill in the art. For example, the expression level of the DNA damage response gene can determined by measuring the expression level of the DNA damage response gene using one or more polynucleotide probes, each of which comprises a nucleotide sequence in the DNA damage response gene. In particularly preferred embodiments of the invention, the method is used to diagnose resistance of a cancer to a treatment using DNA damaging agent in a human.

DNA may be used in hybridization or amplification assays of biological samples to detect abnormalities involving DNA damage response gene structure, including point mutations, insertions, deletions and chromosomal rearrangements. Such assays may include, but are not limited to, Southern analyses, single stranded conformational polymorphism analyses (SSCP), DNA microarray analyses, and PCR analyses.

Such diagnostic methods for the detection of DNA damage response gene-specific mutations can involve, for example, contacting and incubating nucleic acids including recombinant DNA molecules, cloned genes or degenerate variants thereof, obtained from a sample, e.g., derived from a patient sample or other appropriate cellular source, with one or more labeled nucleic acid reagents including recombinant DNA molecules, cloned genes or degenerate variants thereof, under conditions favorable for the specific annealing of these reagents to their complementary sequences within the DNA damage response gene. Preferably, the lengths of these nucleic acid reagents are at least 15 to 30 nucleotides. After incubation, all non-annealed nucleic acids are removed from the nucleic acid:DNA damage response molecule hybrid. The presence of nucleic acids which have hybridized, if any such molecules exist, is then detected. Using such a detection scheme, the nucleic acid from the cell type or tissue of interest can be immobilized, for example, to a solid support such as a membrane, or a plastic surface such as that on a microtiter plate or polystyrene beads. In this case, after incubation, non-annealed, labeled nucleic acid reagents are easily removed. Detection of the remaining, annealed, labeled DNA damage response nucleic acid reagents is accomplished using standard techniques well-known to those in the art. The DNA damage response gene sequences to which the nucleic acid reagents have annealed can be compared to the annealing pattern expected from a normal DNA damage response gene sequence in order to determine whether a DNA damage response gene mutation is present.

Alternative diagnostic methods for the detection of DNA damage response gene specific nucleic acid molecules, in patient samples or other appropriate cell sources, may involve their amplification, e.g., by PCR (the experimental embodiment set forth in Mullis, K. B., 1987, U.S. Pat. No. 4,683,202), followed by the detection of the amplified molecules using techniques well known to those of skill in the art. The resulting amplified sequences can be compared to those which would be expected if the nucleic acid being amplified contained only normal copies of the DNA damage response gene in order to determine whether a DNA damage response gene mutation exists.

Among the DNA damage response nucleic acid sequences which are preferred for such hybridization and/or PCR analyses are those which will detect the presence of the DNA damage response gene splice site mutation.

Additionally, well-known genotyping techniques can be performed to identify individuals carrying DNA damage response gene mutations. Such techniques include, for example, the use of restriction fragment length polymorphisms (RFLPs), which involve sequence variations in one of the recognition sites for the specific restriction enzyme used.

Additionally, improved methods for analyzing DNA polymorphisms which can be utilized for the identification of DNA damage response gene mutations have been described which capitalize on the presence of variable numbers of short, tandemly repeated DNA sequences between the restriction enzyme sites. For example, Weber (U.S. Pat. No. 5,075,217, which is incorporated herein by reference in its entirety) describes a DNA marker based on length polymorphisms in blocks of (dC-dA)n-(dG-dT)n short tandem repeats. The average separation of (dC-dA)n-(dG-dT)n blocks is estimated to be 30,000-60,000 bp. Markers which are so closely spaced exhibit a high frequency co-inheritance, and are extremely useful in the identification of genetic mutations, such as, for example, mutations within the DNA damage response gene, and the diagnosis of diseases and disorders related to DNA damage response mutations.

Also, Caskey et al. (U.S. Pat. No. 5,364,759, which is incorporated herein by reference in its entirety) describe a DNA profiling assay for detecting short tri and tetra nucleotide repeat sequences. The process includes extracting the DNA of interest, such as the DNA damage response gene, amplifying the extracted DNA, and labelling the repeat sequences to form a genotypic map of the individual's DNA.

The level of DNA damage response gene expression can also be assayed. For example, RNA from a cell type or tissue known, or suspected, to express the DNA damage response gene, such as a cancer cell type which exhibits DNA damaging agent resistance, may be isolated and tested utilizing hybridization or PCR techniques such as are described, above. The isolated cells can be derived from cell culture or from a patient. The analysis of cells taken from culture may be a necessary step in the assessment of cells to be used as part of a cell-based gene therapy technique or, alternatively, to test the effect of compounds on the expression of the DNA damage response gene. Such analyses may reveal both quantitative and qualitative aspects of the expression pattern of the DNA damage response gene, including activation or inactivation of DNA damage response gene expression.

In one embodiment of such a detection scheme, a cDNA molecule is synthesized from an RNA molecule of interest (e.g., by reverse transcription of the RNA molecule into cDNA). A sequence within the cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method are chosen from among the DNA damage response gene nucleic acid reagents. The preferred lengths of such nucleic acid reagents are at least 9-30 nucleotides. For detection of the amplified product, the nucleic acid amplification may be performed using radioactively or non-radioactively labeled nucleotides. Alternatively, enough amplified product may be made such that the product may be visualized by utilizing any suitable nucleic acid staining method, e.g., by standard ethidium bromide staining.

Additionally, it is possible to perform such DNA damage response gene expression assays “in situ”, i.e., directly upon tissue sections (fixed and/or frozen) of patient tissue obtained from biopsies or resections, such that no nucleic acid purification is necessary. Nucleic acids from a DNA damage response gene may be used as probes and/or primers for such in situ procedures (see, for example, Nuovo, G. J., 1992, “PCR In Situ Hybridization:

Protocols And Applications”, Raven Press, NY).

Alternatively, if a sufficient quantity of the appropriate cells can be obtained, standard Northern analysis can be performed to determine the level of mRNA expression of the DNA damage response gene.

The expression of DNA damage response gene in cells or tissues, e.g., the cellular level of DNA damage response transcripts and/or the presence or absence of mutations, can also be evaluated using DNA microarray technologies. In such technologies, one or more polynucleotide probes each comprising a sequence of the DNA damage response gene are used to monitor the expression of the DNA damage response gene. The present invention therefore provides DNA microarrays comprising polynucleotide probes comprising sequences of the DNA damage response gene.

Any formats of DNA microarray technologies can be used in conjunction with the present invention. In one embodiment, spotted cDNA arrays are prepared by depositing PCR products of cDNA fragments, e.g., full length cDNAs, ESTs, etc., of the DNA damage response gene onto a suitable surface (see, e.g., DeRisi et al., 1996, Nature Genetics 14:457-460; Shalon et al., 1996, Genome Res. 6:689-645; Schena et al., 1995, Proc. Natl. Acad. Sci. U.S.A. 93:10539-11286; and Duggan et al., Nature Genetics Supplement 21:10-14). In another embodiment, high-density oligonucleotide arrays containing oligonucleotides complementary to sequences of DNA damage response gene are synthesized in situ on the surface by photolithographic techniques (see, e.g., Fodor et al., 1991, Science 251:767-773; Pease et al., 1994, Proc. Natl. Acad. Sci. U.S.A. 91:5022-5026; Lockhart et al., 1996, Nature Biotechnology 14:1675; McGall et al., 1996, Proc. Natl. Acad. Sci. U.S.A. 93:13555-13560; U.S. Pat. Nos. 5,578,832; 5,556,752; 5,510,270; 5,858,659; and 6,040,138). This format of microarray technology is particular useful for detection of single nucleotide polymorphisms (SNPs) (see, e.g., Hacia et al., 1999, Nat Genet. 22:164-7; Wang et al., 1998, Science 280:1077-82). In yet another embodiment, high-density oligonucleotide arrays containing oligonucleotides complementary to sequences of DNA damage response gene are synthesized in situ on the surface by inkjet technologies (see, e.g., Blanchard, International Patent Publication WO 98/41531, published Sep. 24, 1998; Blanchard et al., 1996, Biosensors and Bioelectronics 11:687-690; Blanchard, 1998, in Synthetic DNA Arrays in Genetic Engineering, Vol. 20, J. K. Setlow, Ed., Plenum Press, New York at pages 111-123).

In still another embodiment, DNA microarrays that allow electronic stringency control can be used in conjunction with polynucleotide probes comprising sequences of the DNA damage response gene (see, e.g., U.S. Pat. No. 5,849,486).

### 5.4.5.2. Detection of DNA Damage Response Gene Products

Antibodies directed against wild type or mutant DNA damage response gene products or conserved variants or peptide fragments thereof may be used as diagnostics and prognostics of DNA damaging agent resistance, as described herein. Such diagnostic methods may be used to detect abnormalities in the level of DNA damage response gene expression, or abnormalities in the structure and/or temporal, tissue, cellular, or subcellular location of DNA damage response gene product.

Because evidence disclosed herein indicates that the DNA damage response gene product is an intracellular gene product, the antibodies and immunoassay methods described below have important in vitro applications in assessing the efficacy of treatments for disorders resulting from defective regulation of DNA damage response gene such as proliferative diseases. Antibodies, or fragments of antibodies, such as those described below, may be used to screen potentially therapeutic compounds in vitro to determine their effects on DNA damage response gene expression and DNA damage response peptide production. The compounds which have beneficial effects on disorders related to defective regulation of DNA damage response can be identified, and a therapeutically effective dose determined.

In vitro immunoassays may also be used, for example, to assess the efficacy of cell-based gene therapy for disorders related to defective regulation of DNA damage response. Antibodies directed against DNA damage response peptides may be used in vitro to determine the level of DNA damage response gene expression achieved in cells genetically engineered to produce DNA damage response peptides. Given that evidence disclosed herein indicates that the DNA damage response gene product is an intracellular gene product, such an assessment is, preferably, done using cell lysates or extracts. Such analysis will allow for a determination of the number of transformed cells necessary to achieve therapeutic efficacy in vivo, as well as optimization of the gene replacement protocol.

The tissue or cell type to be analyzed will generally include those which are known, or suspected, to express the DNA damage response gene, such as, a DNA damaging agent resistant cancer cell type. The protein isolation methods employed herein may, for example, be such as those described in Harlow and Lane (Harlow, E. and Lane, D., 1988, “Antibodies: A Laboratory Manual”, Cold Spring Harbor Laboratory Press, Cold Spring Harbor, New York), which is incorporated herein by reference in its entirety. The isolated cells can be derived from cell culture or from a patient. The analysis of cell taken from culture may be used to test the effect of compounds on the expression of the DNA damage response gene.

Preferred diagnostic methods for the detection of DNA damage response gene products or conserved variants or peptide fragments thereof, may involve, for example, immunoassays wherein the DNA damage response gene products or conserved variants or peptide fragments are detected by their interaction with an anti-DNA damage response gene product-specific antibody.

For example, antibodies, or fragments of antibodies, that bind DNA damage response protein, may be used to quantitatively or qualitatively detect the presence of DNA damage response gene products or conserved variants or peptide fragments thereof. This can be accomplished, for example, by immunofluorescence techniques employing a fluorescently labeled antibody (see below, this Section) coupled with light microscopic, flow cytometric, or fluorimetric detection. Such techniques are especially preferred if such DNA damage response gene products are expressed on the cell surface.

The antibodies (or fragments thereof) useful in the present invention may, additionally, be employed histologically, as in immunofluorescence or immunoelectron microscopy, for in situ detection of DNA damage response gene products or conserved variants or peptide fragments thereof. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the DNA damage response gene product, or conserved variants or peptide fragments, but also its distribution in the examined tissue. Using the present invention, those of ordinary skill will readily perceive that any of a wide variety of histological methods (such as staining procedures) can be modified in order to achieve such in situ detection.

Immunoassays for DNA damage response gene products or conserved variants or peptide fragments thereof will typically comprise incubating a sample, such as a biological fluid, a tissue extract, freshly harvested cells, or lysates of cells which have been incubated in cell culture, in the presence of a detectably labeled antibody capable of identifying DNA damage response gene products or conserved variants or peptide fragments thereof, and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled DNA damage response protein specific antibody. The solid phase support may then be washed with the buffer a second time to remove unbound antibody. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tub, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of anti-DNA damage response gene product antibody may be determined according to well known methods. Those skilled in the art will be able to determine operative and optimal assay conditions for each determination by employing routine experimentation.

One of the ways in which the DNA damage response gene peptide-specific antibody can be detectably labeled is by linking the same to an enzyme and use in an enzyme immunoassay (EIA) (Voller, A., “The Enzyme Linked Immunosorbent Assay (ELISA)”, 1978, Diagnostic Horizons 2:1-7, Microbiological Associates Quarterly Publication, Walkersville, Md.); Voller, A. et al., 1978, J. Clin. Pathol. 31:507-520; Butler, J. E., 1981, Meth. Enzymol. 73:482-523; Maggio, E. (ed.), 1980, Enzyme Immunoassay, CRC Press, Boca Raton, Fla.; Ishikawa, E. et al., (eds.), 1981, Enzyme Immunoassay, Kgaku Shoin, Tokyo). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by calorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect DNA damage response gene peptides through the use of a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody can also be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

### 5.4.6. Methods of Regulating Expression of DNA Damage Response Gene

A variety of therapeutic approaches may be used in accordance with the invention to modulate expression of the DNA damage response gene in vivo. For example, siRNA molecules may be engineered and used to silence DNA damage response gene in vivo. Antisense DNA molecules may also be engineered and used to block translation of DNA damage response mRNA in vivo. Alternatively, ribozyme molecules may be designed to cleave and destroy the DNA damage response mRNAs in vivo. In another alternative, oligonucleotides designed to hybridize to the 5′ region of the DNA damage response gene (including the region upstream of the coding sequence) and form triple helix structures may be used to block or reduce transcription of the DNA damage response gene. Oligonucleotides can also be designed to hybridize and form triple helix structures with the binding site of a negative regulator so as to block the binding of the negative regulator and to enhance the transcription of the DNA damage response gene.

In a preferred embodiment, siRNA, antisense, ribozyme, and triple helix nucleotides are designed to inhibit the translation or transcription of one or more of DNA damage response isoforms with minimal effects on the expression of other genes that may share one or more sequence motif with a DNA damage response. To accomplish this, the oligonucleotides used should be designed on the basis of relevant sequences unique to DNA damage response.

For example, and not by way of limitation, the oligonucleotides should not fall within those region where the nucleotide sequence of DNA damage response is most homologous to that of other genes. In the case of antisense molecules, it is preferred that the sequence be chosen from the list above. It is also preferred that the sequence be at least 18 nucleotides in length in order to achieve sufficiently strong annealing to the target mRNA sequence to prevent translation of the sequence. Izant et al., 1984, Cell, 36:1007-1015; Rosenberg et al., 1985, Nature, 313:703-706.

In the case of the “hammerhead” type of ribozymes, it is also preferred that the target sequences of the ribozymes be chosen from the list above. Ribozymes are RNA molecules which possess highly specific endoribonuclease activity. Hammerhead ribozymes comprise a hybridizing region which is complementary in nucleotide sequence to at least part of the target RNA, and a catalytic region which is adapted to cleave the target RNA. The hybridizing region contains nine (9) or more nucleotides. Therefore, the hammerhead ribozymes of the present invention have a hybridizing region which is complementary to the sequences listed above and is at least nine nucleotides in length. The construction and production of such ribozymes is well known in the art and is described more fully in Haseloff et al., 1988, Nature, 334:585-591.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO 88/04300 by University Patents Inc.; Been et al., 1986, Cell, 47:207-216). The Cech endoribonucleases have an eight base pair active site which hybridizes to a target RNA sequence whereafter cleavage of the target RNA takes place.

In the case of oligonucleotides that hybridize to and form triple helix structures at the 5′ terminus of the DNA damage response gene and can be used to block transcription, it is preferred that they be complementary to those sequences in the 5′ terminus of DNA damage response which are not present in other DNA damage response related genes. It is also preferred that the sequences not include those regions of the DNA damage response promoter which are even slightly homologous to that of other DNA damage response related genes. The foregoing compounds can be administered by a variety of methods which are known in the art including, but not limited to the use of liposomes as a delivery vehicle. Naked DNA or RNA molecules may also be used where they are in a form which is resistant to degradation such as by modification of the ends, by the formation of circular molecules, or by the use of alternate bonds including phosphothionate and thiophosphoryl modified bonds. In addition, the delivery of nucleic acid may be by facilitated transport where the nucleic acid molecules are conjugated to poly-lysine or transferrin. Nucleic acid may also be transported into cells by any of the various viral carriers, including but not limited to, retrovirus, vaccinia, AAV, and adenovirus.

Alternatively, a recombinant nucleic acid molecule which encodes, or is, such antisense, ribozyme, triple helix, or DNA damage response molecule can be constructed. This nucleic acid molecule may be either RNA or DNA. If the nucleic acid encodes an RNA, it is preferred that the sequence be operatively attached to a regulatory element so that sufficient copies of the desired RNA product are produced. The regulatory element may permit either constitutive or regulated transcription of the sequence. In vivo, that is, within the cells or cells of an organism, a transfer vector such as a bacterial plasmid or viral RNA or DNA, encoding one or more of the RNAs, may be transfected into cells e.g. (Llewellyn et al., 1987, J. Mol. Biol., 195:115-123; Hanahan et al. 1983, J. Mol. Biol., 166:557-580). Once inside the cell, the transfer vector may replicate, and be transcribed by cellular polymerases to produce the RNA or it may be integrated into the genome of the host cell. Alternatively, a transfer vector containing sequences encoding one or more of the RNAs may be transfected into cells or introduced into cells by way of micromanipulation techniques such as microinjection, such that the transfer vector or a part thereof becomes integrated into the genome of the host cell.

RNAi can also be used to knock down the expression of DNA damage response. In one embodiment, double-stranded RNA molecules of 21-23 nucleotides which hybridize to a homologous region of mRNAs transcribed from the DNA damage response gene are used to degrade the mRNAs, thereby “silence” the expression of the DNA damage response gene. Preferably, the dsRNAs have a hybridizing region, e.g., a 19-nucleotide double-stranded region, which is complementary to a sequence of the coding sequence of the DNA damage response gene. Any siRNA targeting an appropriate coding sequence of a DNA damage response gene, e.g., a human DNA damage response gene, can be used in the invention. As an exemplary embodiment, 21-nucleotide double-stranded siRNAs targeting the coding regions of DNA damage response gene are designed according to standard selection rules (see, e.g., Elbashir et al., 2002, Methods 26:199-213, which is incorporated herein by reference in its entirety).

Any standard method for introducing nucleic acids into cells can be used. In one embodiment, gene silencing is induced by presenting the cell with the siRNA targeting the DNA damage response gene (see, e.g., Elbashir et al., 2001, Nature 411, 494-498; Elbashir et al., 2001, Genes Dev. 15, 188-200, all of which are incorporated by reference herein in their entirety). The siRNAs can be chemically synthesized, or derived from cleavage of double-stranded RNA by recombinant Dicer. Another method to introduce a double stranded DNA (dsRNA) for silencing of the DNA damage response gene is shRNA, for short hairpin RNA (see, e.g., Paddison et al., 2002, Genes Dev. 16, 948-958; Brummelkamp et al., 2002, Science 296, 550-553; Sui, G. et al. 2002, Proc. Natl. Acad. Sci. USA 99, 5515-5520, all of which are incorporated by reference herein in their entirety). In this method, an siRNA targeting DNA damage response gene is expressed from a plasmid (or virus) as an inverted repeat with an intervening loop sequence to form a hairpin structure. The resulting RNA transcript containing the hairpin is subsequently processed by Dicer to produce siRNAs for silencing. Plasmid-based shRNAs can be expressed stably in cells, allowing long-term gene silencing in cells both in vitro and in vivo (see, McCaffrey et al. 2002, Nature 418, 38-39; Xia et al., 2002, Nat. Biotech. 20, 1006-1010; Lewis et al., 2002, Nat. Genetics 32, 107-108; Rubinson et al., 2003, Nat. Genetics 33, 401-406; Tiscomia et al., 2003, Proc. Natl. Acad. Sci. USA 100, 1844-1848, all of which are incorporated by reference herein in their entirety). SiRNAs targeting the DNA damage response gene can also be delivered to an organ or tissue in a mammal, such a human, in vivo (see, e.g., Song et al. 2003, Nat. Medicine 9, 347-351; Sorensen et al., 2003, J. Mol. Biol. 327, 761-766; Lewis et al., 2002, Nat. Genetics 32, 107-108, all of which are incorporated by reference herein in their entirety). In this method, a solution of siRNA is injected intravenously into the mammal. The siRNA can then reach an organ or tissue of interest and effectively reduce the expression of the target gene in the organ or tissue of the mammal.

### 5.4.7. Methods of Regulating Activity of a DNA Damage Response Protein and/or Its Pathway

The activity of DNA damage response protein can be regulated by modulating the interaction of DNA damage response protein with its binding partners. In one embodiment, agents, e.g., antibodies, aptamers, small organic or inorganic molecules, can be used to inhibit binding of a DNA damage response binding partner such that DNA damaging agent resistance is regulated. In another embodiment, agents, e.g., antibodies, aptamers, small organic or inorganic molecules, can be used to inhibit the activity of a protein in a DNA damage response protein regulatory pathway such that DNA damaging agent resistance is regulated. In one embodiment, a kinase inhibitor, e.g., Herbimycin, Gleevec, Genistein, Lavendustin, Iressa, is used to regulate the activety of DNA damage response protein kinases.

### 5.4.8. Cancer Therapy by Targeting a DNA Damage Response Gene and/or Its Product

The methods and/or compositions described above for modulating DNA damage response expression and/or activity may be used to treat patients who have a cancer in conjunction with a DNA damaging agent. In particular, the methods and/or compositions may be used in conjunction with a DNA damaging agent for treatment of a patient having a cancer which exhibits DNA damage response mediated DNA damaging agent resistance. Such therapies may be used to treat cancers, including but not limted to, rhabdomyosarcoma, neuroblastoma and glioblastoma, small cell lung cancer, osteoscarcoma, pancreatic cancer, breast and prostate cancer, murine melanoma and leukemia, and B-cell lymphoma.

In preferred embodiments, the methods and/or compositions of the invention are used in conjunction with a DNA damaging agent for treatment of a patient having a cancer which exhibits DNA damage response mediated DNA damaging agent resistance. In such embodiments, the expression and/or activity of DNA damage response are modulated to confer cancer cells sensitivity to a DNA damaging agent, thereby conferring or enhancing the efficacy of DNA damaging agent therapy.

In a combination therapy, one or more compositions of the present invention can be administered before, at the same time of, or after the administration of a DNA damaging agent. In one embodiment, the compositions of the invention are administered before the administration a DNA damaging agent. The time intervals between the administration of the compositions of the invention and a DNA damaging agent can be determined by routine experiments that are familiar to one skilled person in the art. In one embodiment, a DNA damaging agent is given after the DNA damage response protein level reaches a desirable threshold. The level of DNA damage response protein can be determined by using any techniques described supra.

In another embodiment, the compositions of the invention are administered at the same time with the DNA damaging agent.

In still another embodiment, one or more of the compositions of the invention are also administered after the administration of a DNA damaging agent. Such administration can be beneficial especially when the DNA damaging agent has a longer half life than that of the one or more of the compositions of the invention used in the treatment.

It will be apparent to one skilled person in the art that any combination of different timing of the administration of the compositions of the invention and a DNA damaging agent can be used. For example, when the DNA damaging agent has a longer half life than that of the composition of the invention, it is preferable to administer the compositions of the invention before and after the administration of the DNA damaging agent.

The frequency or intervals of administration of the compositions of the invention depends on the desired DNA damage response level, which can be determined by any of the techniques described supra. The administration frequency of the compositions of the invention can be increased or decreased when the DNA damage response protein level changes either higher or lower from the desired level.

The effects or benefits of administration of the compositions of the invention alone or in conjunction with a DNA damaging agent can be evaluated by any methods known in the art, e.g., by methods that are based on measuring the survival rate, side effects, dosage requirement of the DNA damaging agent, or any combinations thereof. If the administration of the compositions of the invention achieves any one or more of the benefits in a patient, such as increasing the survival rate, decreasing side effects, lowing the dosage requirement for the DNA damaging agent, the compositions of the invention are said to have augmented the DNA damaging agent therapy, and the method is said to have efficacy.

### 5.5. Pharmaceutical Formulations and Routes of Administration

The compounds that are determined to affect STK6 gene expression or gene product activity can be administered to a patient at therapeutically effective doses to treat or ameliorate disorders related to defective regulation of STK6. A therapeutically effective dose refers to that amount of the compound sufficient to result in amelioration of KSPi resistance and/or enhancement of the growth inhibitory effect of a KSP inhibitor in cells.

### 5.5.1. Effective Dose

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### 5.5.2. Formulations and Use

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

### 5.5.3. Routes of Administration

Suitable routes of administration may, for example, include oral, rectal, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections.

Alternately, one may administer the compound in a local rather than systemic manner, for example, via injection of the compound directly into an affected area, often in a depot or sustained release formulation.

Furthermore, one may administer the drug in a targeted drug delivery system, for example, in a liposome coated with an antibody specific for affected cells. The liposomes will be targeted to and taken up selectively by the cells.

### 5.5.4. Packaging

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition. Suitable conditions indicated on the label may include treatment of a disease such as one characterized by aberrant or excessive STK6 or a DNA damage response gene expression or activity.

### 6. Examples

The following examples are presented by way of illustration of the present invention, and are not intended to limit the present invention in any way.

### 6.1. Example 1

### STK6 and TPX2 Interacts with KSP

This Example illustrates screening of an siRNA library for genes that interact with inhibitors of KSP gene. CIN8 is the S. cerevisiae homolog of KSP. Deletion mutants of CIN8 are viable and many genes have been identified that are essential in the absence (but not the presence) of CIN8 (Geiser et al., 1997, Mol Biol Cell. 8:1035-1050). By analogy, it was reasoned that disruption of human homologues of these genes might be more disruptive to tumor cell growth in the presence than in the absence of suboptimal concentrations of a KSPi. An siRNA library containing siRNAs to homologues of 11 genes reported to be synthetic lethal with CIN8: CDC20, ROCK2, TTK, FZR1, BUB1, BUB3, BUB1B, MAD1L1, MAD2L1, DNCH1 and STK6 was screened for genes that modulates the effect of a KSP inhibitor, (1S)-1-{[(2S)-4-(2,5-difluorophenyl)-2-phenyl-2,5-dihydro-1H-pyrrol-1-yl]carbonyl}-2-methylpropylamine, (EC50˜80 nM). The sequences of siRNAs targeting the 11 genes are listed in Table I. These siRNAs were transfected into HeLa cells in the presence or absence of an <EC10 concentration (25 nM) of (1S)-1-{[(2S)-4-(2,5-difluorophenyl)-2-phenyl-2,5-dihydro-1H-pyrrol-1-yl]carbonyl}-2-methylpropylamine. Table I also lists the sequences of siRNAs that target respectively luciferase, PTEN, and KSP.

siRNA transfection was carried out as follows: one day prior to transfection, 100 microliters of a chosen cells, e.g., cervical cancer HeLa cells (ATCC, Cat. No. CCL-2), grown in DMEM/10% fetal bovine serum (Invitrogen, Carlsbad, Calif.) to approximately 90% confluency were seeded in a 96-well tissue culture plate (Corning, Corning, N.Y.) at 1500 cells/well. For each transfection 85 microliters of OptiMEM (Invitrogen) was mixed with 5 microliter of serially diluted siRNA (Dharmacon, Denver) from a 20 micro molar stock. For each transfection 5 microliter of OptiMEM was mixed with 5 microliter of Oligofectamine reagent (Invitrogen) and incubated 5 minutes at room temperature. The 10-microliter OptiMEM/Oligofectamine mixture was dispensed into each tube with the OptiMEM/siRNA mixture, mixed and incubated 15-20 minutes at room temperature. 10 microliter of the transfection mixture was aliquoted into each well of the 96-well plate and incubated for 4 hours at 37° C. and 5% CO₂.

After 4 hours, 100 microliter/well of DMEM/10% fetal bovine serum with or without 25 nM (1S)-1-{[(2S)-4-(2,5-difluorophenyl)-2-phenyl-2,5-dihydro-1H-pyrrol-1-yl]carbonyl}-2-methylpropylamine was added and the plates were incubated at 37° C. and 5% CO₂ for 68 hours. The alamarBlue Assay was used for measurement of cell growth (see, Section 5.2). The alamarBlue assay measures cellular respiration and uses the meausrement as a measure of the number of living cells. The internal environment of the proliferating cell is more reduced than that of non-proliferating cells. Specifically, the ratios of NADPH/NADP, FADH/FAD, FMNH/FMN, and NADH/NAF increase during proliferation. AlamarBlue can be reduced by these metabolic intermediates and, therefore, can be used to monitor cell proliferation. In this Example, the alamarBlue assay was performed to determine whether STK6 siRNA transfection titration curves were changed by the presence of 25 nM of the KSP inhibitor (1S)-1-{[(2S)-4-(2,5-difluorophenyl)-2-phenyl-2,5-dihydro-1H-pyrrol-1-yl]carbonyl}-2-methylpropylamine as follows: 72 hours after transfection the medium was removed from the wells and replaced with 100 microliter/well DMEM/10% Fetal Bovine Serum (Invitrogen) containing 10% (vouvol) alamarBlue reagent (Biosource International Inc., Camarillo, Calif.) and 0.001 volumes of 1M Hepes buffer tissue culture reagent (Invitrogen). The plates were incubated 2 hours at 37° C. and the plate was read at 570 and 600 nm wavelengths on a SpectraMax plus plate reader (Molecular Devices, Sunnyvale, Calif.) using Softmax Pro 3.1.2 software (Molecular Devices). The % Reduced of wells containing samples was determined according to Eq. 1. The % Reduced of the wells containing no cell was subtracted from the % Reduced of the wells containing samples to determine the % Reduced above the background level. The % Reduced for wells transfected with a titration of STK6 siRNA with or without 25 nM (1S)-1-{[(2S)-4-(2,5-difluorophenyl)-2-phenyl-2,5-dihydro-1H-pyrrol-1-yl]carbonyl}-2-methylpropylamine were compared to that of wells transfected with an siRNA targeting luciferase. The number calculated for % Reduced for 0 nM luciferase siRNA-transfected wells without (1S)-1-{[(2S)-4-(2,5-difluorophenyl)-2-phenyl-2,5-dihydro-1H-pyrrol-1-yl]carbonyl}-2-methylpropylamine was considered to be 100%.

Three siRNAs targeting STK6 (STK6-1, STK6-2, and STK6-3) showed inhibition of tumor cell growth in the presence of (1S)-1-{[(2S)-4-(2,5-difluorophenyl)-2-phenyl-2,5-dihydro-1H-pyrrol-1-yl]carbonyl}-2-methylpropylamine. Among the three, STK6-1 showed the strongest growth inhibitory activity in the initial screens. To investigate whether this growth inhibitory activity was due to on or off-target activity of the siRNA, three additional siRNAs targeting STK6 were used and the abilities of all six siRNAs to induce STK6 silencing and growth inhibition were investigated. There was a good correlation between the level of STK6 silencing and growth inhibition (FIG. 1). This correlation suggested that growth inhibition was due to on target activity (i.e., STK6 disruption). Next, STK6-1 and control siRNAs to luciferase (negative control) were titrated in the presence or absence of (1S)-1-{[(2S)-4-(2,5-difluorophenyl)-2-phenyl-2,5-dihydro-1H-pyrrol-1-yl]carbonyl}-2-methylpropylamine (FIG. 2). The addition of the KSPi shifted the STK6-1 dose response curve ˜5-10-fold to the left. This concentration of the KSPi did not augment effects on cell growth caused by a luciferase siRNA. In contrast, the dose response curve to an siRNA targeting PTEN (Table I) with similar effects on cell growth as STK6-1 was not shifted by the KSPi. Other siRNAs to STK6 also enhanced effects of KSPi on cell growth. Thus, disruption of KSP enhances the effects of STK6 siRNAs on cell growth. Further support for this was obtained by studies using combinations of siRNAs to STK6 and KSP, which showed greater growth inhibitory activity than either siRNAs alone. Because the concentrations of KSPi used in these experiments did not affect cell growth on its own, the effects of KSPi on STK6 siRNA activity appeared synergistic rather than additive.

The interaction between human STK6 and KSP is consistent with evidence of physiological interactions between these genes in Xenopus (Giet et al., 1999, J Biol. Chem. 274:15005-5013). In particular, the Xenopus homologues of STK6 and KSP co-localize at the mitotic spindle poles and the proteins show molecular association by immunoprecipitation. Furthermore, KSP is a substrate for STK6.

The growth inhibition by STK6 siRNAs suggests that this gene is essential for tumor cell growth and supports investigation of STK6 as an anti-tumor target. The data showing synthetic lethal interactions between inhibitors of STK6 and KSPi suggest that combination therapy with these compounds might be more effective than therapy with either compounds alone. STK6 is frequently over-expressed in human tumors, including breast cancers with poor prognosis (van 't Veer et al., 2002, Nature. 2002 415:530-536). Amplification of STK6 has been implicated in resistance to Taxol (Anand et al., 2003, Cancer Cell. 3:51-62). Since both KSPi and Taxol affect the same target (mitotic spindle), over-expression of STK6 may likewise reduces the effectiveness of KSPi. This possibility is consistent with the results showing interactions between inhibitors of KSPi and STK6, and should be investigated during the clinical development of KSPi. For instance, a KSPi may not be optimally effective in breast cancer patients with poor prognosis because of the tendency of these tumors to over-express STK6.

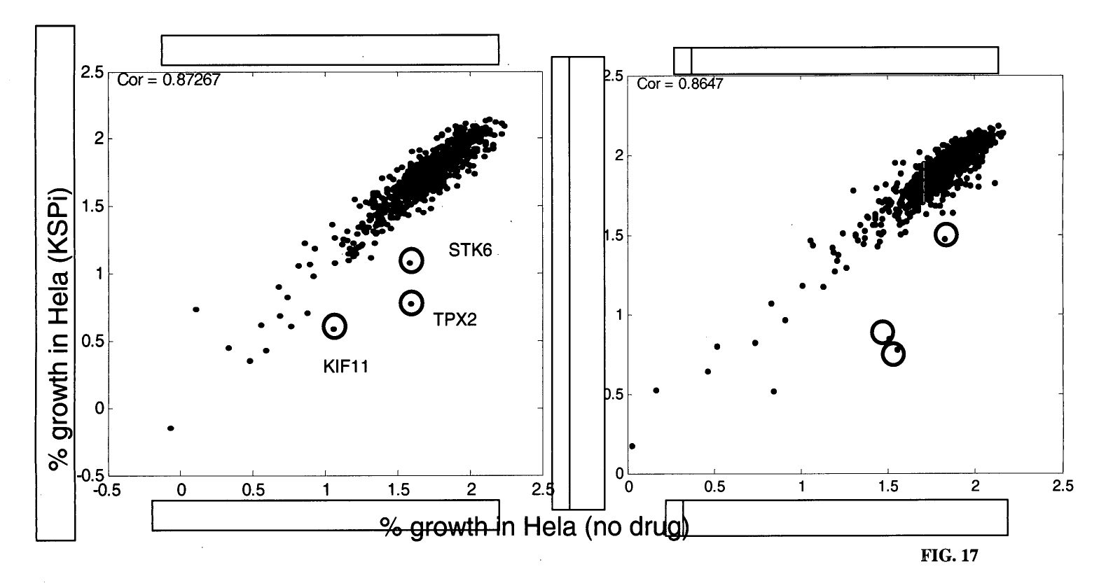

FIG. 17 shows results of screens for genes that sensitize to KSPi. The results demonstrate that TPX2 also interacts with KSP. The siRNA sequences used in silencing TPX2 are also listed in Table I.

TABLE I

List of siRNAs

| | | |
|---|---|---|
| STK6-1 | GCACAAAAGCUUGUCUCCATT | (SEQ ID NO:1) |
| STK6-2 | UUGCAGAUUUUGGGUGGUCTT | (SEQ ID NO:2) |
| STK6-3 | ACAGUCUUAGGAAUCGUGCTT | (SEQ ID NO:3) |
| STK6-4 | CCUCCCUAUUCAGAAAGCUTT | (SEQ ID NO:4) |
| STK6-5 | GACUUUGAAAUUGGUCGCCTT | (SEQ ID NO:5) |
| STK6-6 | CACCCAAAAGAGCAAGCAGTT | (SEQ ID NO:6) |
| ROCK2-1 | AACCAGUCUAUUAGACGGCTT | (SEQ ID NO:7) |
| ROCK2-2 | GUGACUCUCCAUCUUGUAGTT | (SEQ ID NO:8) |
| ROCK2-3 | GUGGCCUCAAAGGCACUUATT | (SEQ ID NO:9) |
| CDC20-1 | CCCAUCACCUCAGUUGUUUTT | (SEQ ID NO:10) |
| CDC20-2 | GACCUGCCGUUACAUUCCUTT | (SEQ ID NO:11) |
| CDC20-3 | GGAGAACCAGUCUGAAAACTT | (SEQ ID NO:12) |
| TTK-1 | AUGCUGGAAAUUGCCCUGCTT | (SEQ ID NO:13) |
| TTK-2 | ACAACCCAGAGGACUGGUUTT | (SEQ ID NO:14) |
| TTK-3 | UAUGUUCUGGGCCAACUUGTT | (SEQ ID NO:15) |
| FZR1-1 | CCAGAUCCUUGUCUGGAAGTT | (SEQ ID NO:16) |
| FZR1-2 | CGACAACAAGCUGCUGGUCTT | (SEQ ID NO:17) |
| FZR1-3 | GAAGCUGUCCAUGUUGGAGTT | (SEQ ID NO:18) |
| BUB1-1 | CUGUAUGGGGUAUUCGCUGTT | (SEQ ID NO:19) |
| BUB1-2 | ACCCAUUUGCCAGCUCAAGTT | (SEQ ID NO:20) |
| BUB1-3 | CAGACUCCAUGUUUGCAGUTT | (SEQ ID NO:21) |
| BUB3-1 | UACAUUUGCCACAGGUGGUTT | (SEQ ID NO:22) |
| BUB3-2 | CAAUUCGUACUCCCCAAUGTT | (SEQ ID NO:23) |
| BUB3-3 | AGCUGCUUCAGACUGCUUCTT | (SEQ ID NO:24) |
| MAD1L1-1 | GACCUUUCCAGAUUCGUGGTT | (SEQ ID NO:25) |
| MAD1L1-2 | AGAGCAGAGCAGAUCCGUUTT | (SEQ ID NO:26) |
| MAD1L1-3 | CCAGCGGCUCAAGGAGGUUTT | (SEQ ID NO:27) |
| MAD2L2-1 | CCAUGACGUCGGACAUUUUTT | (SEQ ID NO:28) |
| MAD2L2-2 | GUGCUCUUAUCGCCUCUGUTT | (SEQ ID NO:29) |
| MAD2L2-3 | ACGCAAGAAGUACAACGUGTT | (SEQ ID NO:30) |
| DNCH1-1 | GCAAGUUGAGCUCUACCGCTT | (SEQ ID NO:31) |
| DNCH1-2 | UGGCCAGCGCUUACUGGAATT | (SEQ ID NO:32) |
| DNCH1-3 | GGCCAAGGAGGCGCUGGAATT | (SEQ ID NO:33) |
| BUB1B-1 | AUGACCCUCUGGAUGUUUGTT | (SEQ ID NO:34) |
| BUB1B-2 | UGCCAAUGAUGAGGCCACATT | (SEQ ID NO:35) |
| BUB1B-3 | GAAAGAACAGGUGAUCAGCTT | (SEQ ID NO:36) |
| Luciferase | CGUACGCGGAAUACUUCGATT | (SEQ ID NO:37) |
| KSP-1 | CUGGAUCGUAAGAAGGCAGTT | (SEQ ID NO:38) |
| KSP-2 | GGACAACUGCAGCUACUCUTT | (SEQ ID NO:39) |
| PTEN-1 | UGGAGGGGAAUGCUCAGAATT | (SEQ ID NO:40) |
| PTEN-2 | UAAAGAUGGCACUUUCCCGTT | (SEQ ID NO:41) |
| PTEN-3 | AAGGCAGCUAAAGGAAGUGTT | (SEQ ID NO:42) |
| TPX2 | UACUUGAAGGUGGGCCCAUTT | (SEQ ID NO:1237) |
| TPX2 | GAAAUCAGUUGCUGAGGGCTT | (SEQ ID NO:1238) |
| TPX2 | ACCUAGGACCGUCUUGCUUTT | (SEQ ID NO:1239) |

### 6.2. Example 2

### Synthetic Lethal Screen Using shRNA and siRNA

This Example illustrates that simultaneous RNAi-mediated silencing of CHEK1 and TP53 leads to synthetic lethality in human tumor cells.

Two problems have limited the potential for synthetic lethal screening using RNAi approaches. First, the demonstration of synthetic lethality requires that a lethal phenotype induced by a defined gene disruption be observed in cells predisposed by a first hit gene loss or mutation but not in cells containing only wild-type alleles or protein. Thus for highly controlled experimentation, it is desirable to assay for synthetic lethality with matched cell line pairs that are isogenic except for the first hit gene disruption. For most of the available tumor cell lines, such matched cell line pairs have not been available. Second, attempts at creating two gene disruptions in cells by use of dual siRNA transfection has been hampered by the observation that siRNAs targeting distinct mRNAs compete with each other, effectively decreasing the efficacy of one or both of the siRNAs used. It is shown in this example that dual RNAi screens can be achieved through the use of stable in vivo delivery of an shRNA disrupting the first hit gene and supertransfection of an siRNA targeting a second gene. This approach provided matched (isogenic) cell line pairs (plus or minus the shRNA) and did not result in competition between the shRNA and siRNA. In this example, clonal cell lines with a primary gene target silenced by stable expression of short hairpin RNAs (shRNAs) were established. Transient transfection (supertransfection) of these clones with siRNAs targeting other genes did not appreciably affect primary target silencing by the shRNA, nor was target silencing by siRNAs affected by shRNAs. This approach was employed to demonstrate synthetic lethality between TP53 (p53), and the checkpoint kinase, CHEK1, in the presence of low concentrations of the DNA-damaging agent doxorubicin.

RNA interference can be achieved by delivery of synthetic double-stranded small interfering RNAs (siRNAs) via transient transfection or by expression within the cell of short hairpin RNAs (shRNAs) from recombinant vectors introduced either transiently or stably integrated into the genome (see, e.g., Paddison et al., 2002, Genes Dev 16:948-958; Sui et al., 2002, Proc Natl Acad Sci USA 99:5515-5520; Yu et al., 2002, Proc Natl Acad Sci USA 99:6047-6052; Miyagishi et al., 2002, Nat Biotechnol 20:497-500; Paul et al., 2002, Nat Biotechnol 20:505-508; Kwak et al., 2003, J Pharmacol Sci 93:214-217; Brummelkamp et al., 2002, Science 296:550-553; Boden et al., 2003, Nucleic Acids Res 31:5033-5038; Kawasaki et al., 2003, Nucleic Acids Res 31:700-707). The pRETRO-SUPER (pRS) vector which encodes a puromycin-resistance marker and drives shRNA expression from an H1 (RNA Pol III) promoter was used. The pRS-TP53 1026 shRNA plasmid was deconvoluted from the NKI library plasmid pool for TP53 by transforming bacteria with the pool and looking for clones containing only the plasmid of interest. The sequences used are as follows: pRS-p53 1026 19mer sequence: GACTCCAGTGGTAATCTAC (SEQ ID NO:43); primers for sequence specific PCR: Forward: GTAGATTACCACTGGAGTC (SEQ ID NO:44), Reverse: CCCTTGAACCTCCTCGTTCGACC (SEQ ID NO:45). Plasmids were identified by sequence specific PCR, and confirmed by sequencing. Stables were generated by transfecting HCT116 cells using FuGENE 6 (Roche) with the pRS-TP53 1026 plasmid. Cells were split into 10 cm dishes plus 1 ug/ml puromycin 48 hours later, and maintained until colonies were evident (5-7 days). Clones were picked into a 96 well plate, maintained in 1 ug/ml puromycin, and tested for knockdown by TaqMan using the TP53 and hGUS Pre-Developed. Assay Reagent (Applied Biosystems). To measure transient knockdown by the pRS-TP53 1026 plasmid, HCT116 cells were transfected using Lipofectamine 2000 (Invitrogen), and RNA harvested 24 hours later. TP53 levels were assessed by TaqMan.

Analysis of multiple puromycin-resistant TP53 shRNA clones (pRS-p53) derived from the colon tumor line HCT116 showed varying levels of target silencing (50% to 96%). FIG. 3 shows the level of TP53 expression in clones A5 and A11, which exhibited the highest levels of silencing. TP53 silencing achieved in these clones exceeded that observed 24 hr after delivery of pRS-p53 into HCT116 cells by transient transfection (FIG. 3). It is possible that transfection efficiency limits the effectiveness of TP53 shRNA in transient assays. Alternatively, cells having greater levels of TP53 silencing gain a growth advantage during clonal growth. With an shRNA that targets STK6 (pRS-STK6: pRS-STK6 2178 19mer sequence: CATTGGAGTCATAGCATGT (SEQ ID NO:46)), a range of silencing in stable clones was also observed. These clones, however, did not achieve as high a degree of silencing observed in the TP53 lines, nor was silencing greater than that achieved in transient assays. This may indicate selection against high level of STK6 silencing because STK6 is an essential gene for tumor cell growth in culture.

To test whether TP53 silencing in HCT116 clone A11 was subject to competition with siRNAs, cells of this clone were supertransfected with a pool of CHEK1-specific siRNAs. CHK1 pool contains the following three siRNAs: CUGAAGAAGCAGUCGCAGUTT (SEQ ID NO:99); AUCGAUUCUGCUCCUCUAGTT (SEQ ID NO:98); and UGCCUGAAAGAGACUUGUGTT (SEQ ID NO:100). This siRNA pool had been found to competitively reduce silencing activity of a TP53 targeted siRNA. siRNAs were transfected using Oligofectamine (Invitrogen) at 10 nM or 100 nM where indicated. For the CHK1 pool, three siRNAs were transfected simultaneously at 33.3 nM each for a total delivery of 100 nM. RNA was harvested 24 hours post transfection and knockdown was assessed by TaqMan analysis using the CHK1 or TP53 and hGUS Pre-Developed Assay Reagent (Applied Biosystems). As shown in FIG. 4A, the shRNA and the siRNA pool did not competitively inhibit silencing of each other's targets. Inhibition by known competitive siRNAs of either a transiently transfected siRNA or a stably expressed shRNA of the same sequence was then assayed. As shown in FIG. 4B, three individual siRNAs targeting KNSL1 (KNSLI 210: GACCUGUGCCUUUUAGAGATT (SEQ ID NO:47); KNSLI 211: GACUUCAUUGACAGUGGCCTT (SEQ ID NO:48); KNSLI 212: AAAGGACAACUGCAGCUACTT (SEQ ID NO:49)) competitively inhibited the silencing achieved by co-transfected siRNA targeting STK6 (left bars). In contrast, silencing by the homologous STK6 shRNA in stably transfected lines was unaffected by supertransfection of the KNSL1 siRNAs, even when the competitor siRNAs were added at ten fold higher concentrations (middle and right bars). These experiments suggested that there was little competition between stably expressed shRNAs and transiently transfected siRNAs. pRS and pRS-p53 HCT116 cells were transiently transfected with siRNA pools for ˜800 genes (see Example 3, infra) and measured effects on cellular growth by Alamar Blue assay. Nearly identical responses to the ˜800 siRNA pools in pRS cells and in pRS-p53 cells with no suggestion of competitive inhibition of silencing were observed.

Next, supertransfection of the CHEK1 siRNA pool into cells stably expressing TP53 shRNAs was evaluated to determine if it could be used to investigate genetic interactions (SL) between these molecules. This interaction has been speculated previously, but definitive demonstration of it has been hampered by lack of reagents or genetic knockouts with adequate specificity to rule out off-target effects. Matched cell lines +/−TP53 expression were generated by selecting stable clones of A549 lung cancer cell lines containing either empty pRS vector or pRS-p53. The latter cells showed >90% silencing of TP53 mRNA. Both cell lines were then supertransfected with either control luciferase siRNA (luc, 100 nM) or the CHEK1 siRNA pool (100 nM total; 33 nM each of 3 siRNAs) and their cell cycle profiles examined with or without exposure to the DNA damaging agent, doxorubicin (Dox, FIG. 5). Cell cycle profiles of pRS-p53 cells were not appreciably different from those of pRS cells in the absence of Dox. Transient transfection of CHEK1 siRNAs also did not affect cell cycle profiles in the absence of Dox. In the presence of Dox, however, pRS-transfected cells exhibited G1 and G2/M arrest as is expected of cells expressing functional TP53. Supertransfection of CHEK1 siRNAs resulted in an override of the G2 checkpoint and an increase in the number of cells blocked at G1. Because the cells retained TP53 function, they stopped in G1 and did not proceed back into S phase.

In contrast, pRS-p53 cells lost the ability to arrest at G1 and arrested primarily at G2 in response Dox treatment, consistent with the role of TP53 in the G1 checkpoint. The cell cycle profile of pRS-p53 cells was unchanged by supertransfection of luc siRNA (FIG. 5). The failure of luc siRNA to cause even partial restoration of the TP53 response (and a corresponding increase in the G1 peak) suggests that there was little competitive inhibition of TP53 silencing and phenotype by this siRNA. Therefore, competitive inhibition of TP53 silencing by the CHEK1 siRNA pool was not expected to exist. Indeed, in response to Dox treatment, pRS-p53 cells transiently transfected with CHEK1 showed profound alterations in their cell cycle profile with large increases in the fraction of cells in S and with sub-G1 (dead cells) amounts of DNA. Similar findings were also observed in pRS and pRS-p53 stably transfected HCT116 cells. Thus, simultaneous disruption of the G1 checkpoint mediated by TP53 and the G2 checkpoint mediated by CHEK1 is lethal to TP53− but not TP53+ tumor cells.

The finding that transfected siRNAs did not competitively inhibit silencing by stably expressed shRNAs was unexpected. It is presently unclear why siRNAs competitively cross inhibit silencing whereas shRNAs and siRNAs do not. It may suggest that these two types of RNAs enter the RNAi pathway at biochemically distinct steps.

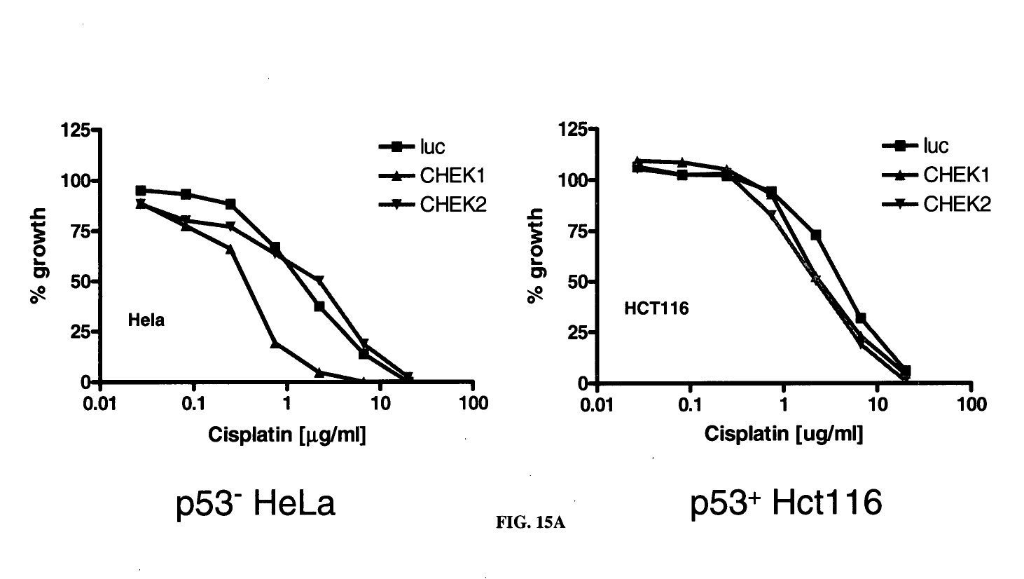

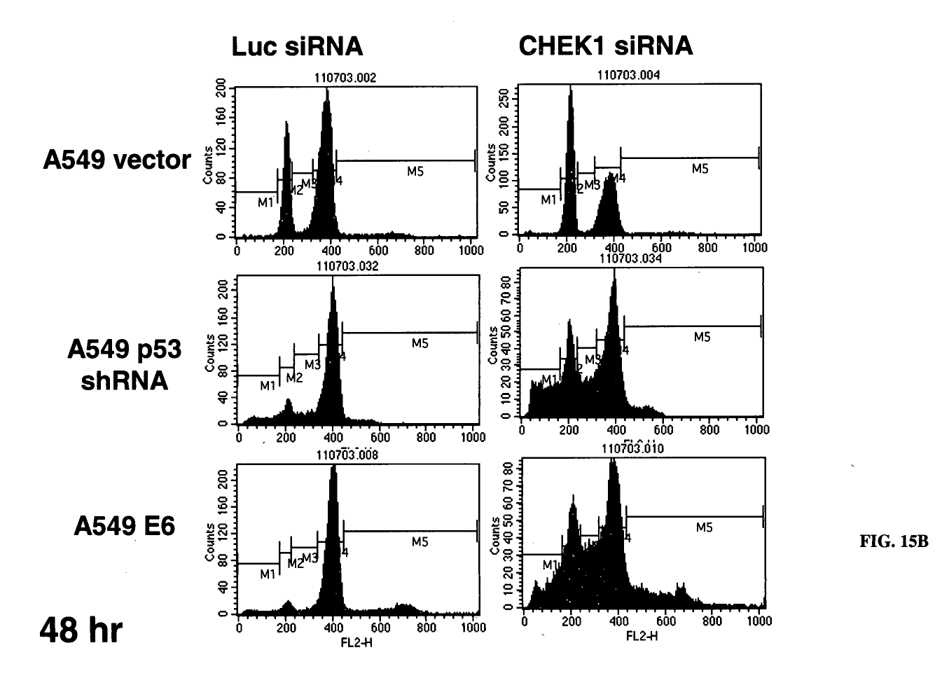

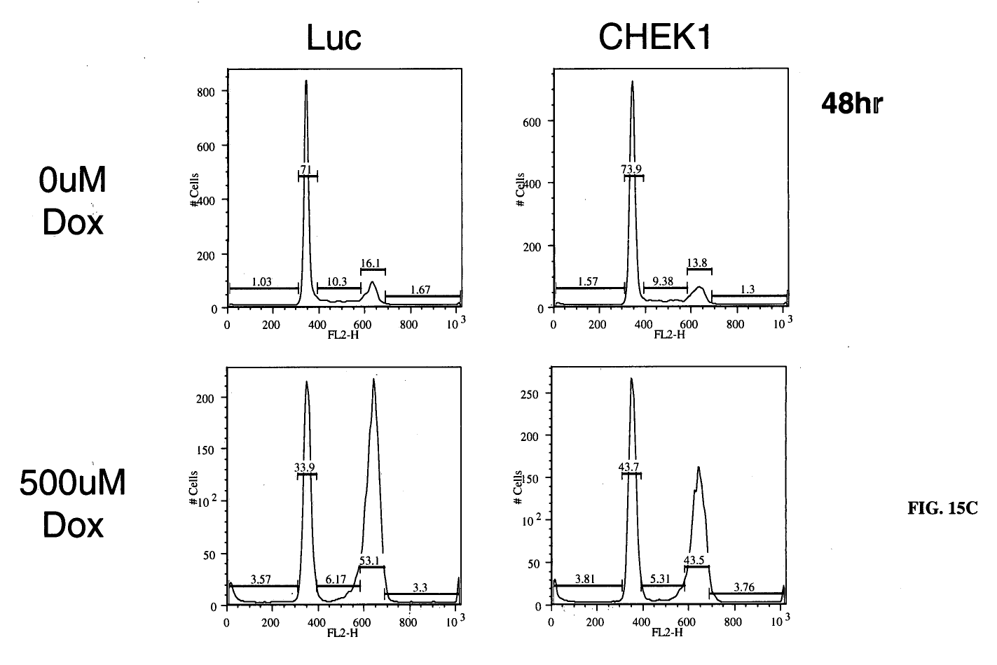

FIGS. 15A-C shows results of CHEK1 silencing on the sensitivity of cells to DNA damage. 15A CHEK1 silencing/inhibition sensitizes HeLa cells to DNA damage. 15B CHEK1 silencing/inhibition sensitizes p53-A549 cells. 15C CHEK1 silencing does not sensitize HREP cells to Doxorubicin.

### 6.3. Example 3

### Genes that Enhance or Reduces Cell Killing by DNA Damaging Agents

This Example illustrates a semi-automated siRNA screens for identification of genes that enhance or reduces cell killing by DNA damaging agents. The semi-automated platform enables loss-of-function RNAi screens using small interfering RNAs (siRNA's). A library of siRNAs targeting ˜800 human genes was used to identify enhancers of DNA damaging agents, Doxorubicin (Dox), Camptothecin (Campto), and Cisplatin (Cis). In each of the screens, many genes (“hits”) whose disruption sensitized cells to cell killing by the chemotherapeutic agent were identified (see Table IIIA-C). Some of these hits (e.g. WEE1) suggest new targets to enhance the activity of common chemotherapeutics; other hits (BRCA1, BRCA2) suggest new therapies for genetically determined cancers caused by mutations in these genes.

The library of siRNA duplexes was assembled for genetic screens in human cells. One version of the library targets ˜800 genes with 3 siRNAs per gene. This library was expended to target ˜2,000 genes, with further expansion to target >7,000 genes (2-3 siRNAs/gene). The library comprises siRNAs that target genes from the “druggable genome” (i.e., genes or gene families that have previously been drugged using small molecules). The library also comprises siRNAs that target genes from the “membraneome” (membrane proteins) to facilitate identification of potential targets for therapeutic antibodies. Tables IIIA-C list the sequences of portions of the siRNAs used in this Example. To facilitate large-scale siRNA screens using the library, a semi-automated platform was developed. Three different siRNAs targeting the same gene were pooled before transfection (100 nM total siRNA concentration). An entire library can be transfected into cells in duplicate by one person in less than 4 hrs. Each siRNA pool was typically tested 2-4 times in a single experiment and each experiment is generally repeated at least twice, usually by different individuals. Excellent reproducibility between screens done on different days or by different persons was achieved.

The goal of the screens was to identify targets that sensitize cells to commonly used cancer chemotherapeutics Dox, Campto, and Cis. Dox (adriamycin) inhibits the activity of topoisomerase II (TopoII). TopoII functions primarily at the G2 and M phases of the cell cycle and is important for resolving DNA structures to allow the proper packing and segregation of chromosomes. Campto inhibits topoisomerase I (TopoI). TopoI functions in S phase to relieve torsional stress of the advancing DNA polymerase complex. The addition of Campto to replicating cells results in stalled replication forks and DNA strand breaks. Cis causes DNA adducts and strand cross-linking. Both Cis and Campto treatments lead to replication fork arrest and possibly fork breakage, leading to dsDNA breaks and cell death.

The primary screen with each agent was performed in HeLa cells, which are TP53 deficient. HeLa cells were transfected with siRNA pools, and the drugs were added 4 hrs later. Preliminary experiments were performed to determine the concentration of each drug used; typically this was the amount required to give 10%-20% growth inhibition (EC10 or EC20). The growth of cells +/−drug was assessed at 72 hrs post-transfection.

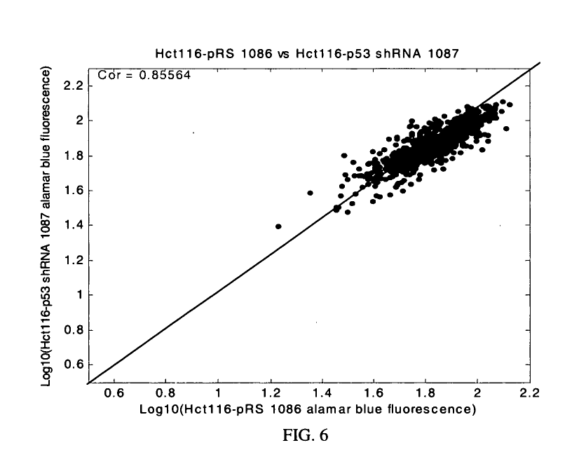

The results of a screen with Cis are shown in FIG. 6. Table IIA shows fold sensitization by cisplatin averaged over cis concentrations of 400 ng/ml and 500 ng/ml. The graph shows the percent growth (log scale) for cells transfected with the siRNA pool in the absence of drug (X axis) versus the percent growth in the presence of drug (Y axis). Genes whose knockdown sensitizes to drug treatment fall below the diagonal whereas genes whose knockdown mediates resistance to the drug fall above the diagonal. The siRNA pool targeting BRCA2 caused >10-fold sensitization to Cis. The siRNA pool to BRCA1 caused >3-fold sensitization. siRNAs targeting kinases WEE1 and EPHB3 also caused >3-fold sensitization to Cis. A total of 15 genes caused >2-fold sensitization. In similar screens, ˜50 genes were identified in each of the Dox and Campto screens that caused >2-fold sensitization to drug (see Table IIB-C). The overlap between the different gene sets is discussed below.

It is important to point out that this screen was designed to reveal enhancers of drug activity. Since the drug concentrations used caused very little effect on cell growth, suppressors of drug activity would also cause very little effect on cell growth. Thus, as expected, we observed very few genes whose disruption suppressed drug activity. The one notable exception was that siRNAs targeting polo-like kinase, PLK, were less active in the presence of Cis. This probably reflects the fact that both DNA damage and PLK disruption cause cell cycle arrest. When cell cycle arrest is induced by the former treatment, the latter treatment is less effective.

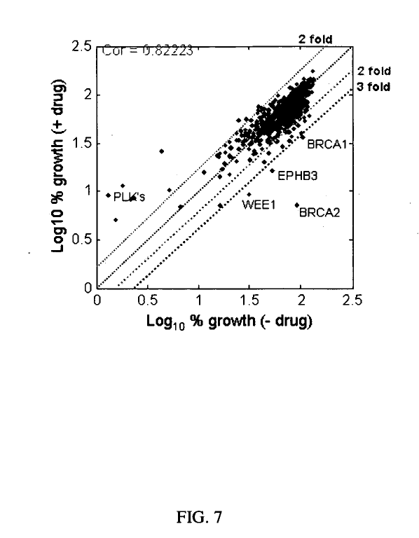

To visualize the overlap between genes causing sensitization to the different drugs, we compared the ratios of cell growth −/+drug (fold sensitization) for the different agents (FIG. 7). Comparison of genes causing sensitization to Dox vs. Cis (FIG. 7, left) revealed that siRNAs to some genes, such as WEE1 kinase, sensitized cells to killing by both agents. In contrast, strong sensitization of cells to killing by Cis (>10 fold) was only observed with siRNAs targeting breast cancer susceptibility gene BRCA2. Comparison of genes causing sensitization to Campto vs. Cis (FIG. 2, right) revealed the same top-scoring genes with both treatments (BRCA2, BRCA1, EPHB3, WEE1, and ELK1).

The observation that WEE1 disruption causes sensitization to all three agents suggests that this kinase regulates a DNA damage response common to all agents. Biochemically, human WEE1 coordinates the transition between DNA replication and mitosis by protecting the nucleus from cytoplasmically activated CDC2 kinase (Heald et al., 1993, Cell 74: 463-474). Other studies suggest that WEE1 is a component of a DNA repair checkpoint functioning during the G2 phase of the cell cycle. Since most human tumors are TP53-deficient, they lack the TP53-regulated checkpoint functioning primarily in G1 and thus are more dependant on other checkpoints than normal tissues that express TP53 (i.e., that have normal checkpoint redundancy). Taken together, available data suggest that WEE1 offer a therapeutic target for treatment of TP53-deficient tumors whose survival is dependent on G2 checkpoint integrity. Indeed, a small molecule inhibitor of WEE1 was reported to act as a radiosensitizer to TP53-deficient cells (i.e., sensitized cells to radiation-induced cell death), although the degree of sensitization conferred by this compound was modest (Wang et al., 2001, Cancer Res. 61:8211-7). The “hits” from these screens in tumor cell checkpoint function are been tested for their ability to sensitize cell killing in other contexts: for example, by use of other DNA damaging agents, in other tumor types, and in matched cells +/−TP53 function.

The overlap in genes sensitizing to Cis and Campto is consistent with the mechanism of action of these drugs. Both target S phase and ultimately stall the progression of replication forks, leading to the formation of dsDNA breaks. In contrast, Dox functions primarily at the G2/M phases of the cell cycle. Thus, sensitization to Campto and Cis by BRCA1 and BRCA2 likely represents an S phase-specific mechanism-based sensitization. These results are consistent with emerging data on the role of BRCA1 and BRCA2 in DNA damage pathways (D'Andrea et al., 2003, Nat Rev Cancer 3:23-34). Indeed, both of these genes are now known to function in the DNA-repair pathway mediated by genes associated with Fanconi anemia; BRCA2 is identical to one of these genes, FANCD1. Cells that harbor defects in the BRCA pathway have an increased sensitivity to Cis (Taniguchi et al., 2003, Nat Med. 9:568-74). At present, cancer patients with BRCA mutations do not receive therapy that targets their genetic defects, although efforts are underway to test platinum drugs in these patients (Couzin, 2003, Science 302:592).

Taken together, these data suggest that the siRNA screens have identified a potential “responder” population for certain DNA damaging agents (i.e., BRCA pathway-deficient tumors). Until recently, it was thought that only a small fraction of breast and ovarian tumors were caused by germline mutations in BRCA genes, as sporadic tumors generally do not manifest alterations in these genes. However, recent data indicate that gene inactivation of other members of the BRCA pathway may be more widespread within sporadic tumors than alterations in the BRCA genes themselves (Marsit et al., 2004, Oncogene 23:1000-4). Future siRNA screens using larger libraries may help identify other genes whose disruption in tumors is diagnostic of sensitivity t6 DNA damaging agents. Indeed, many known and predicted DNA repair genes are represented in the expanded siRNA library (e.g., including other Fanconi anemia genes in the BRCA pathway). Appropriately designed screens may also identify other molecular targets that could benefit patients having BRCA pathway gene disruptions in their tumors.

The primary screens were carried out as follows: the siRNA library containing siRNAs to approximately 800 genes was screened for genes that modulate the effect of Cisplatin (cis-Diaminedichloroplatinum). The library was screened using pools of siRNAs (pool of 3 siRNA per gene) at 100 nM (each siRNA at 33 nM). These siRNAs were transfected into HeLa cells in the presence or absence of an <EC25 concentration (400 ng/ml) of Cisplatin.

siRNA transfection was carried out as follows: one day prior to transfection, 50 microliters of a chosen cell line, e.g., cervical cancer HeLa cells (ATCC, Cat. No. CCL-2), grown in DMEM/10% fetal bovine serum (Invitrogen, Carlsbad, Calif.) to approximately 90% confluency were seeded in a 384-well tissue culture plate at 450 cells/well. For each transfection 20 microliters of OptiMEM (Invitrogen) was mixed with 2 microliter of siRNA (Dharmacon, Lafayette, Colo.) from a 10 micromolar stock. For each transfection, a ratio of 20 microliter of OptiMEM was mixed with 1 microliter of Oligofectamine reagent (Invitrogen) and incubated 5 minutes at room temperature. Then 20-microliter OptiMEM/Oligofectamine mixture was dispensed into each well of the 96 well plate with the OptiMEM/siRNA mixture, mixed and incubated 15-20 minutes at room temperature. 5 microliter of the transfection mixture was aliquoted into each well of the 384-well plate and incubated for 4 hours at 37° C. and 5% CO₂. Four different 96 well plates containing different siRNA pools were distributed at one plate per quadrant of a 384 well plate. All liquid transfers were performed using a BioMek FX liquid handler with a 96 well dispense head.

After 4 hours, 5 microliter/well of DMEM/10% fetal bovine serum with or without 2400 ng/ml of Cisplatin was added and the plates were incubated at 37° C. and 5% CO₂ for 68 hours. The alamarBlue Assay was used for measurement of cell growth (see, Section 5.4.2.2). The alamarBlue assay measures cellular respiration and uses the meausrement as a measure of the number of living cells. The internal environment of the proliferating cell is more reduced than that of non-proliferating cells. Specifically, the ratios of NADPH/NADP, FADH/FAD, FMNH/FMN, and NADH/NAF increase during proliferation. AlamarBlue can be reduced by these metabolic intermediates and, therefore, can be used to monitor cell proliferation. At 72 hours after transfection the medium was removed from the wells and replaced with 50 microliter/well DMEM/10% Fetal Bovine Serum (Invitrogen) containing 10% (vol/vol) alamarBlue reagent (Biosource International Inc., Camarillo, Calif.) and 0.001 volumes of 1M Hepes buffer tissue culture reagent (Invitrogen). The plates were incubated 2 hours at 37° C. and the plate was read by fluorescence with excitation at 545 nm and emission at 590 on a Gemini EM microplate reader (Molecular Devices, Sunnyvale, Calif.) using Softmax Pro 3.1.2 software (Molecular Devices). The relative fluorescence units of the wells containing no cells were subtracted from the relative fluorescence units of the wells transfected with different siRNA pools to determine the relative fluorescence units above the background level. The relative fluorescence units for wells transfected with a siRNA pools with or without Cisplatin were compared to that of wells transfected with an siRNA targeting luciferase. The relative fluorescence units for luciferase siRNA-transfected wells with or without Cisplatin were considered to be 100%. A compare plot was generated by plotting the % growth relative to luciferase in the absence of drug on the X axis versus the the % growth relative to luciferase in the presence of drug on the Y axis.

The secondary screening was carried out using HeLa cells, A549-pRS cells and A549-C7 cells. The cells were transfected using pools of siRNAs (pool of 3 siRNA per gene) at 100 nM (each siRNA at 33 nM), or with single siRNA at 100 nM. These siRNAs were transfected into HeLa cells in the presence or absence of varying concentrations of DNA damaging agents. The concentration for each agent is as following: for HeLa cells, Doxorubicin (10 nM), Camptothecin (6 nM), Cisplatin (500 ng/ml); for the other cell lines, Doxorubicin (200 nM), Camptothecin (200 nM), Cisplatin (4 ug/ml).

The following siRNAs were employed: WEE1 pool, EPHB3 pool, CHUK pool, BRCA1 pool, BRCA2 pool, and STK6. The sequences of the siRNAs used are listed in Table IIIA.

siRNA transfection was carried out as follows: one day prior to transfection, 2000 microliters of a chosen cell line, e.g., cervical cancer HeLa cells (ATCC, Cat. No. CCL-2), grown in DMEM/10% fetal bovine serum (Invitrogen, Carlsbad, Calif.) to approximately 90% confluency were seeded in a 6-well tissue culture plate at 45,000 cells/well. For each transfection 70 microliters of OptiMEM (Invitrogen) was mixed with 5 microliter of siRNA (Dharmacon, Lafayette, Colo.) from a 20 micromolar stock. For each transfection, a ratio of 20 microliter of OptiMEM was mixed with 1 microliter of Oligofectamine reagent (Invitrogen) and incubated 5 minutes at room temperature. Then 25-microliter OptiMEM/Oligofectamine mixture was mixed with the 75-microliter of OptiMEM/siRNA mixture, and incubated 15-20 minutes at room temperature. 100 microliter of the transfection mixture was aliquoted into each well of the 6-well plate and incubated for 4 hours at 37° C. and 5% CO₂.

After 4 hours, 100 microliter/well of DMEM/10% fetal bovine serum with or without DNA damage agents was added to each well to reach the final concentration of each agents as described above. The plates were incubated at 37° C. and 5% CO₂ for another 44 or 68 hours. Samples from the two time points (48 hr or 72 hr post-transfection) were then analyzed for cell cycle profiles.

For cell cycle analysis, the supernatant from each well was combined with the cells that were harvested by trypsinization. The mixture was then centrifuged at 1200 rpm for 5 minutes. The cells were then fixed with ice cold 70% ethanol for ˜30 minutes. Fixed cells were washed once with PBS and resuspended in 0.5 ml of PBS containing Propidium Iodide (10 microgram/ml) and RNase A(1 mg/ml), and incubated at 37° C. for 30 min. Flow cytometric analysis was carried out using a FACSCalibur flow cytometer (Becton Dickinson) and the data was analyzed using FlowJo software (Tree Star, Inc). The Sub-G1 cell population was used to measure cell death. The siRNAs are said to sensitize cells to DNA damage if the summation of the Sub-G1 population from the (siRNA+DMSO) sample and (Luc+drug) sample is larger than the Sub-G1 population of (siRNA+drug) sample.

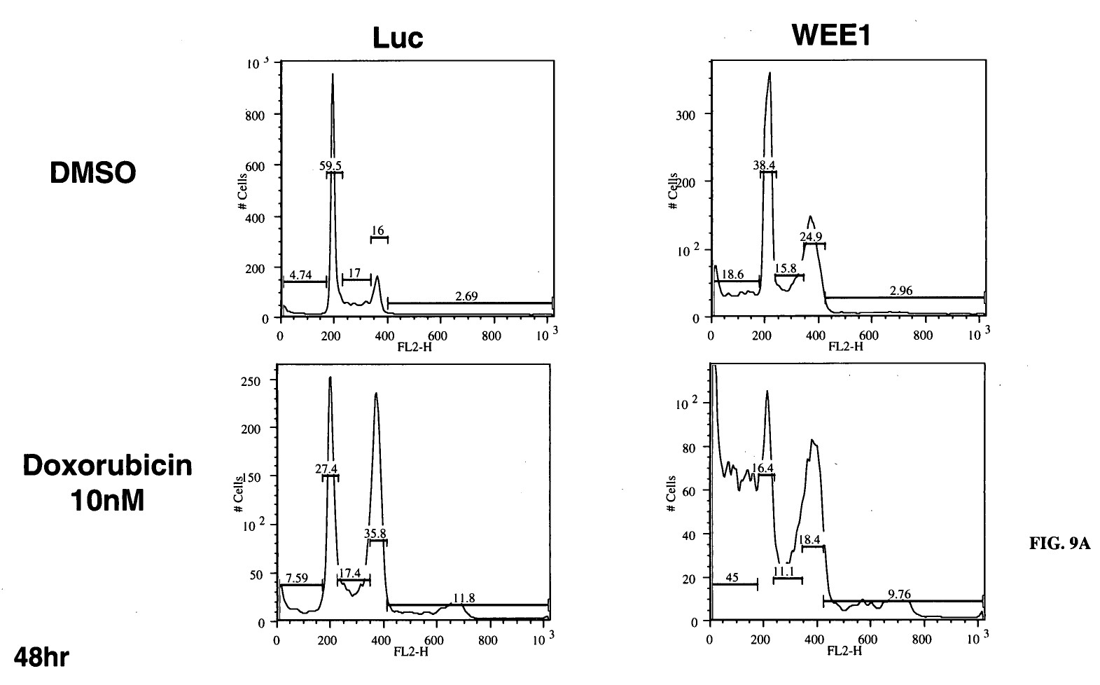

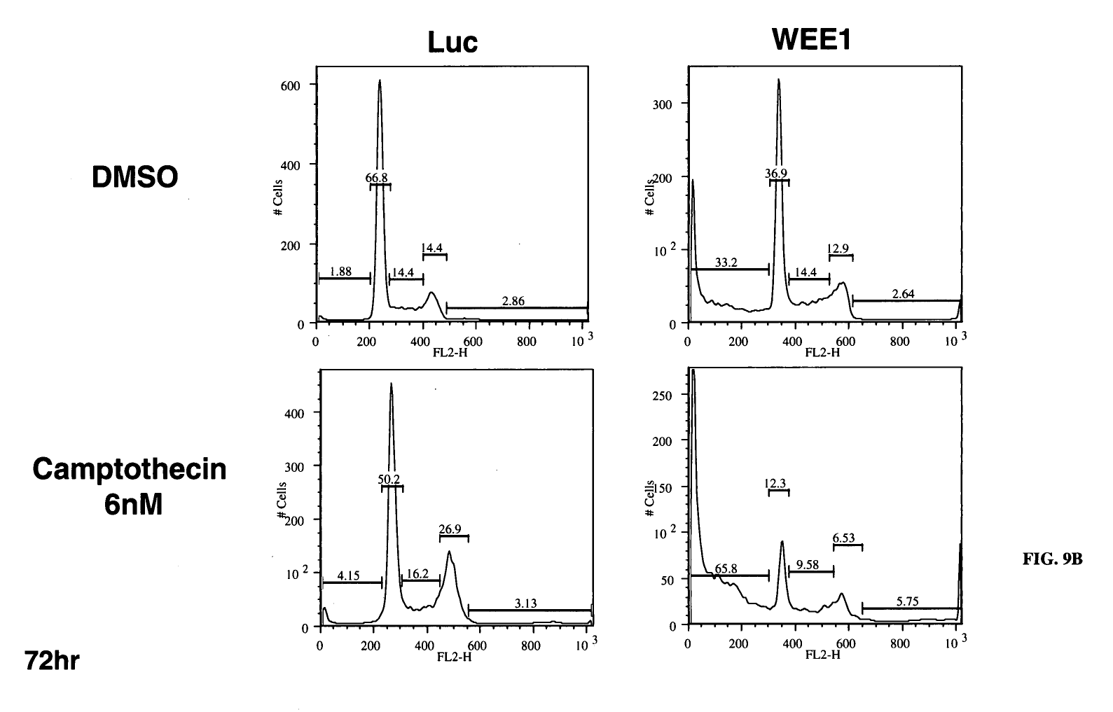

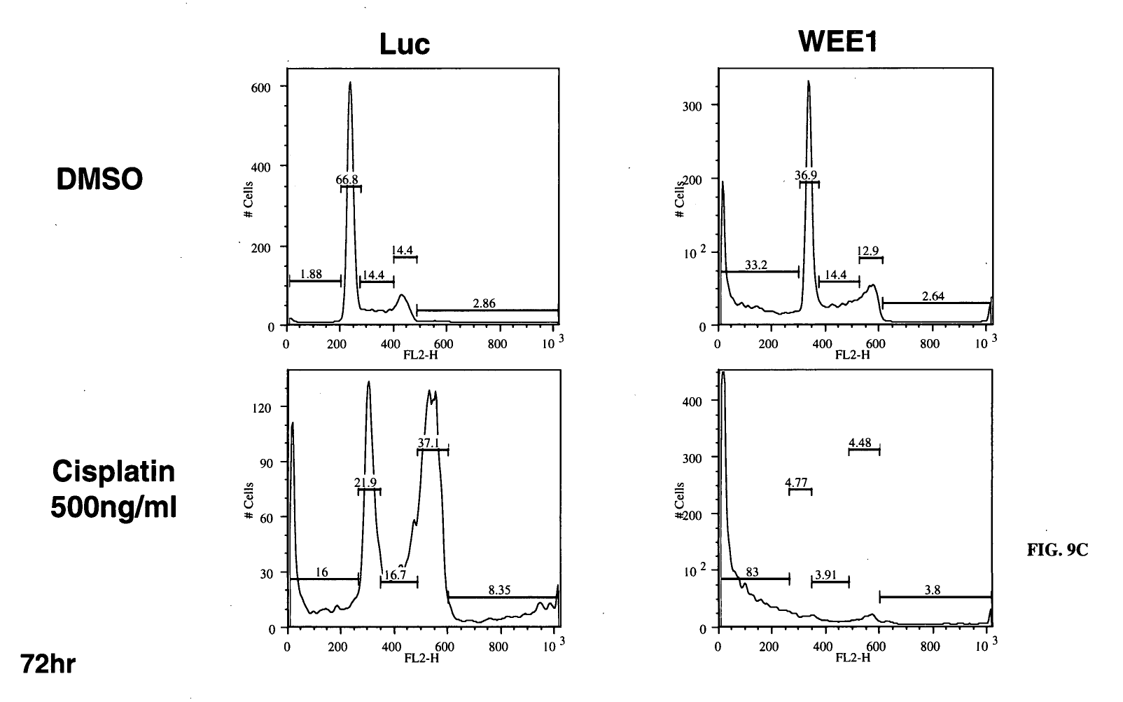

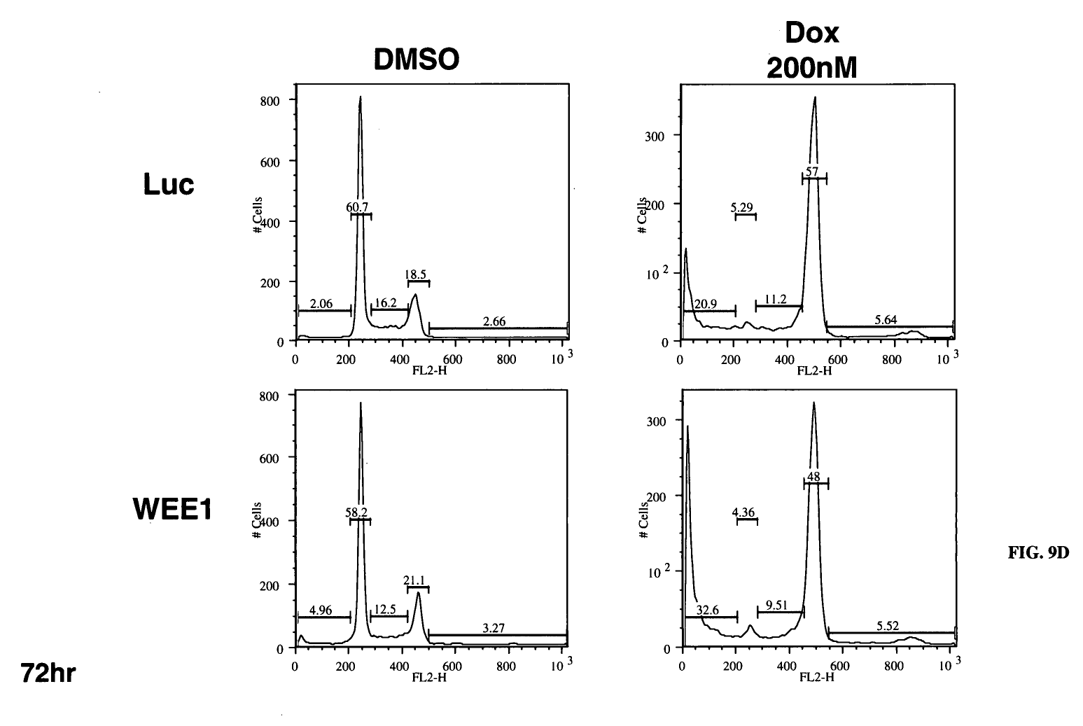

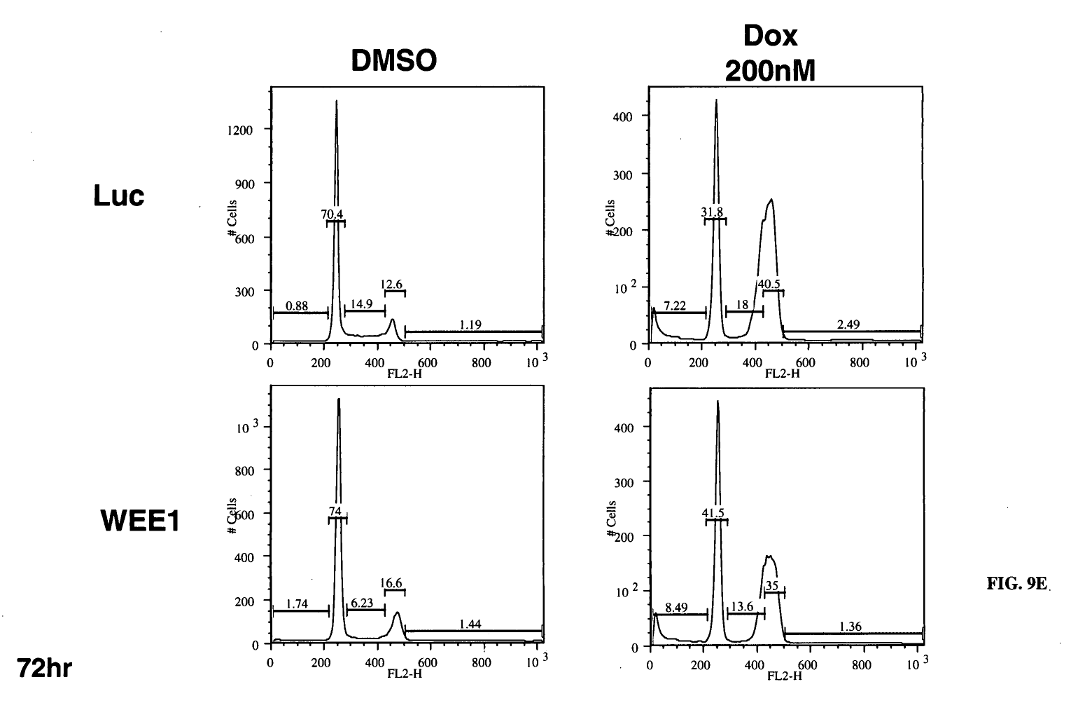

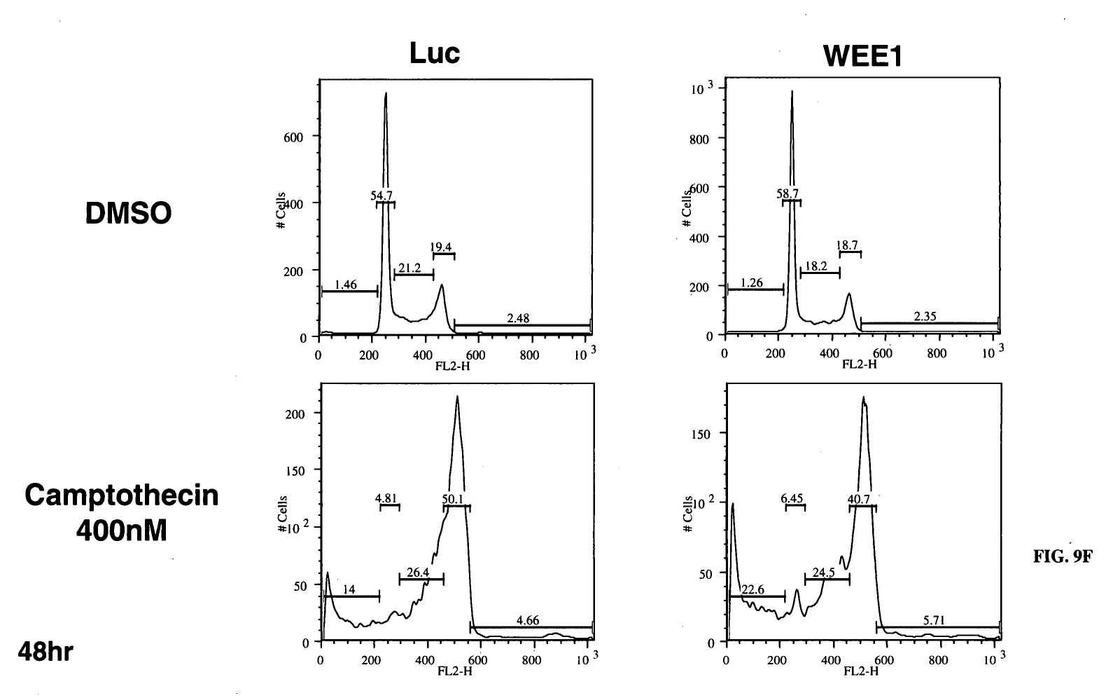

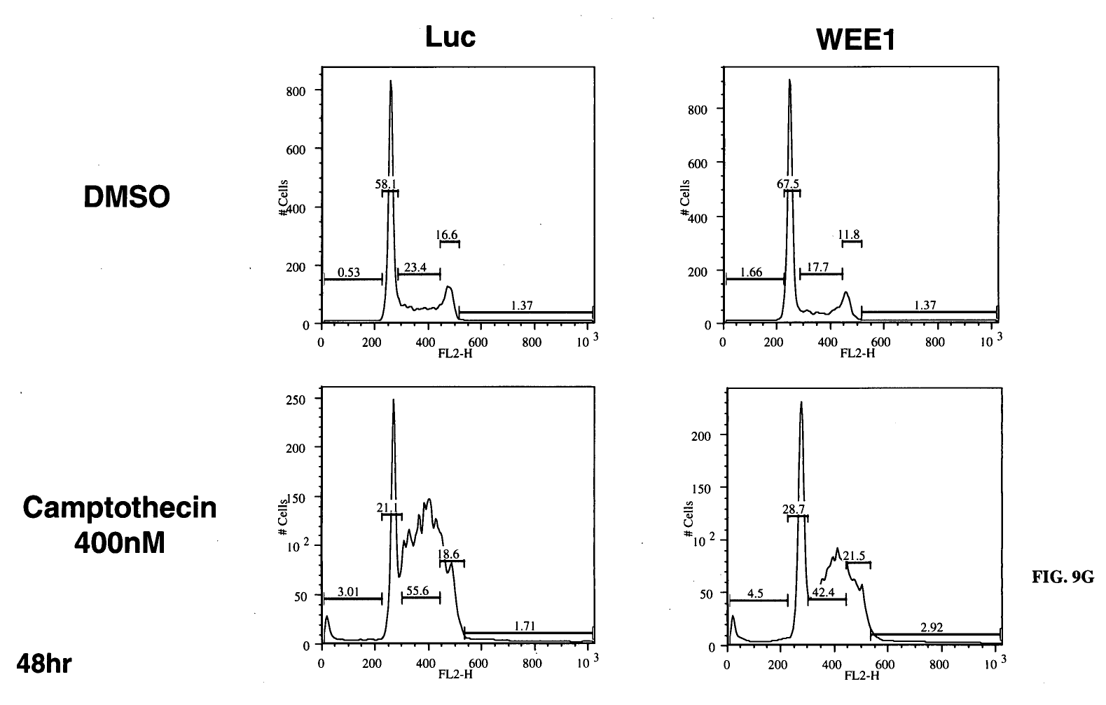

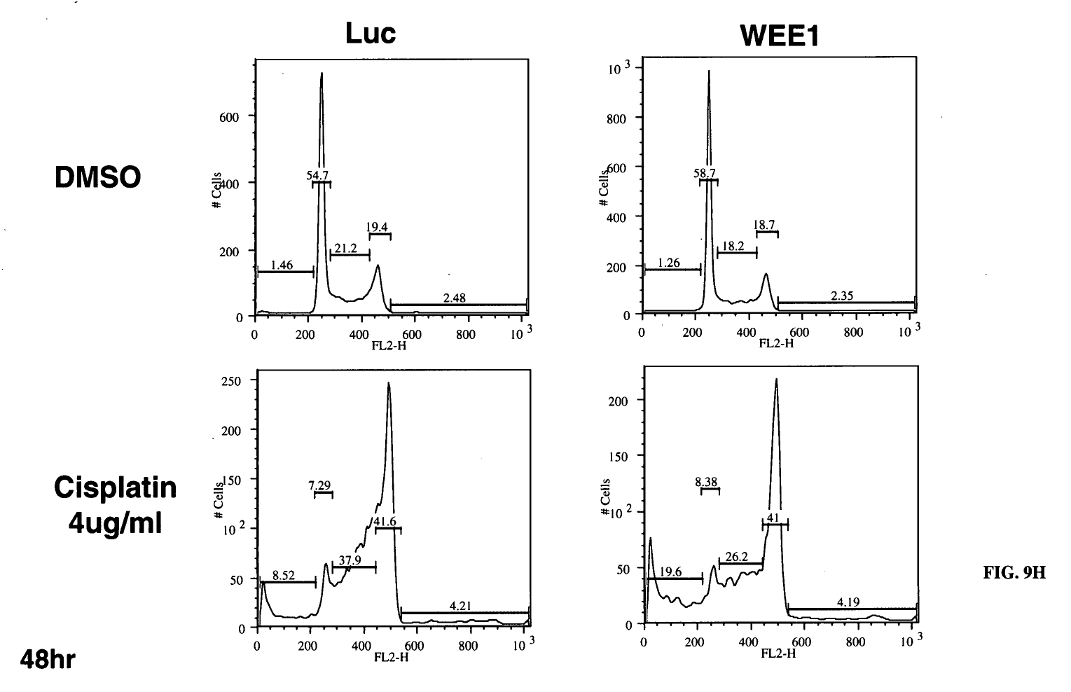

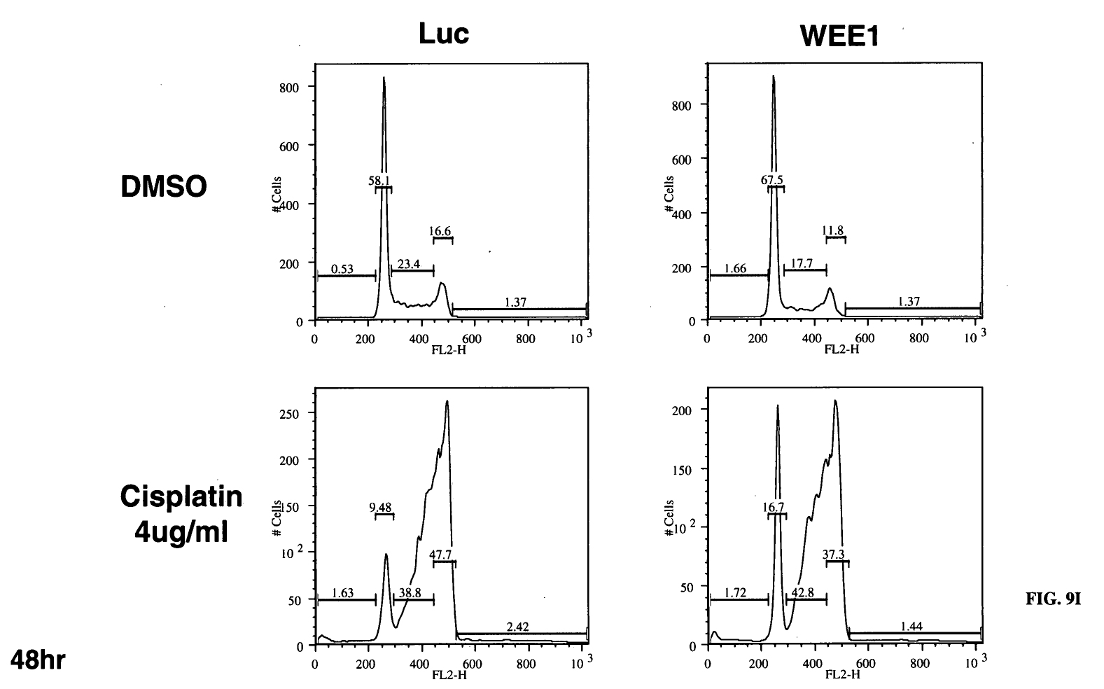

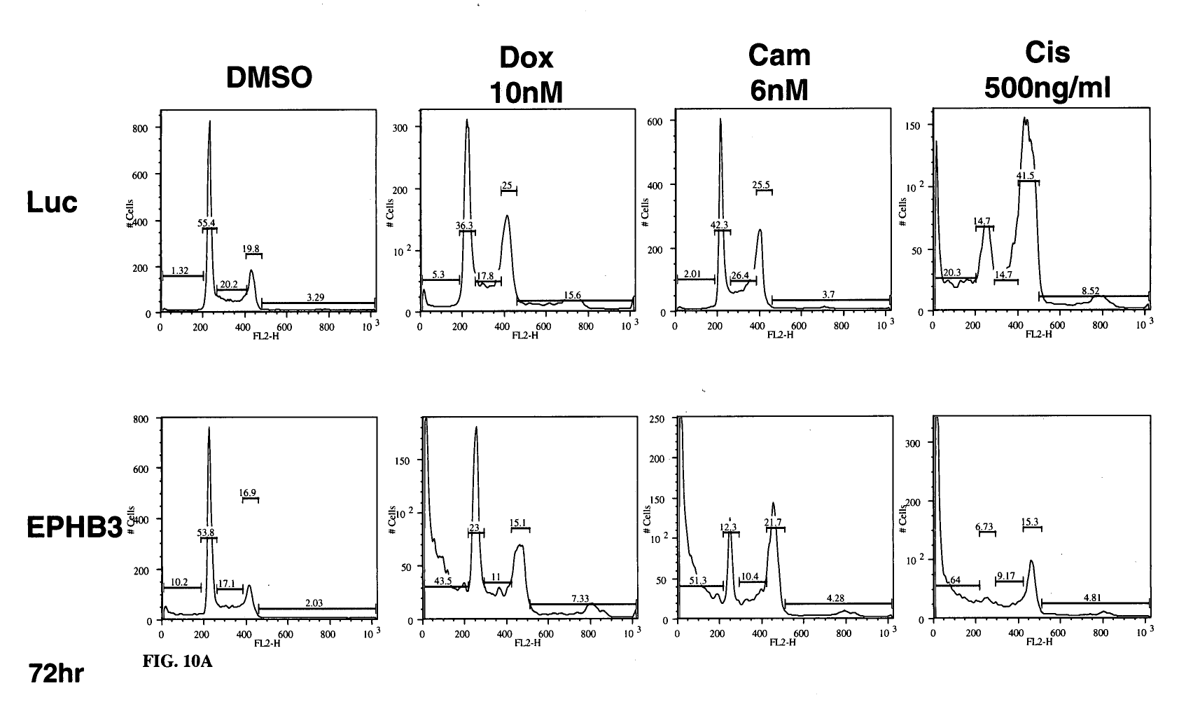

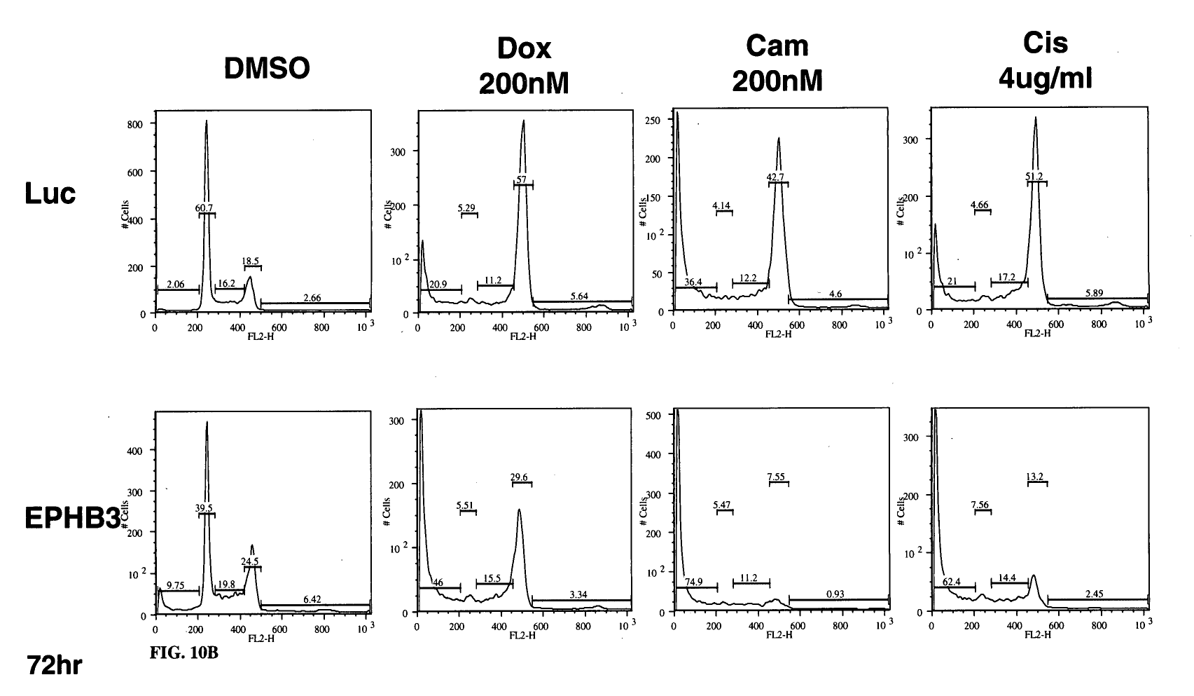

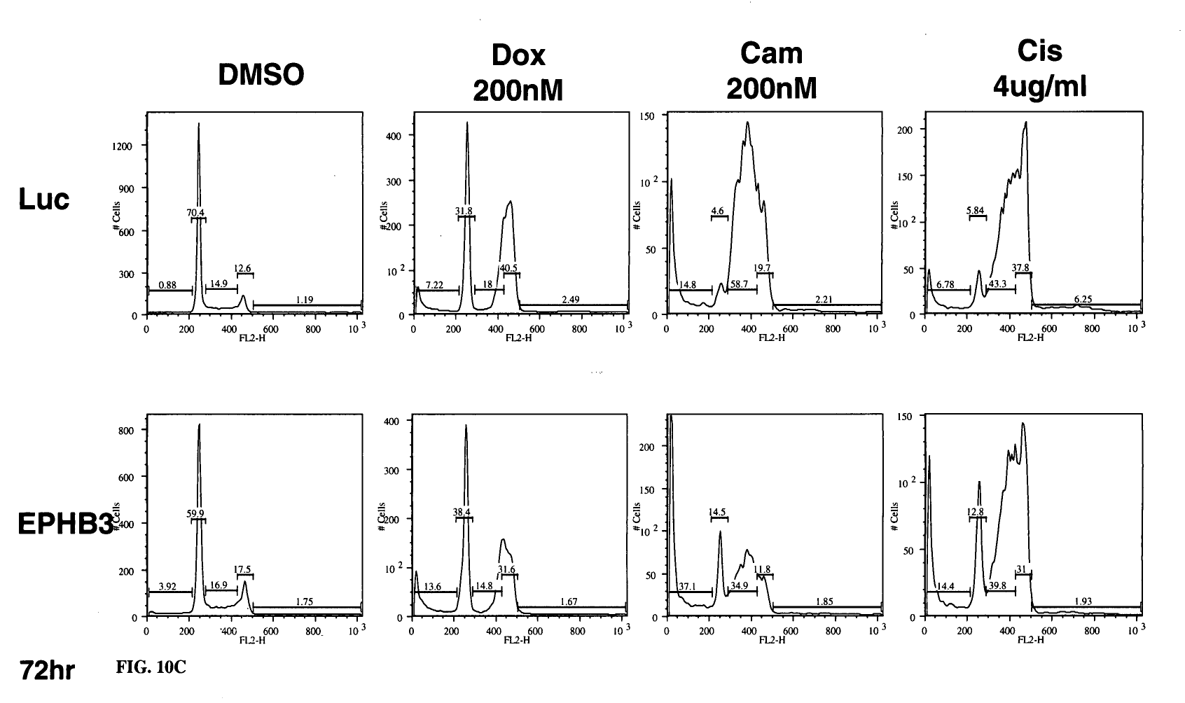

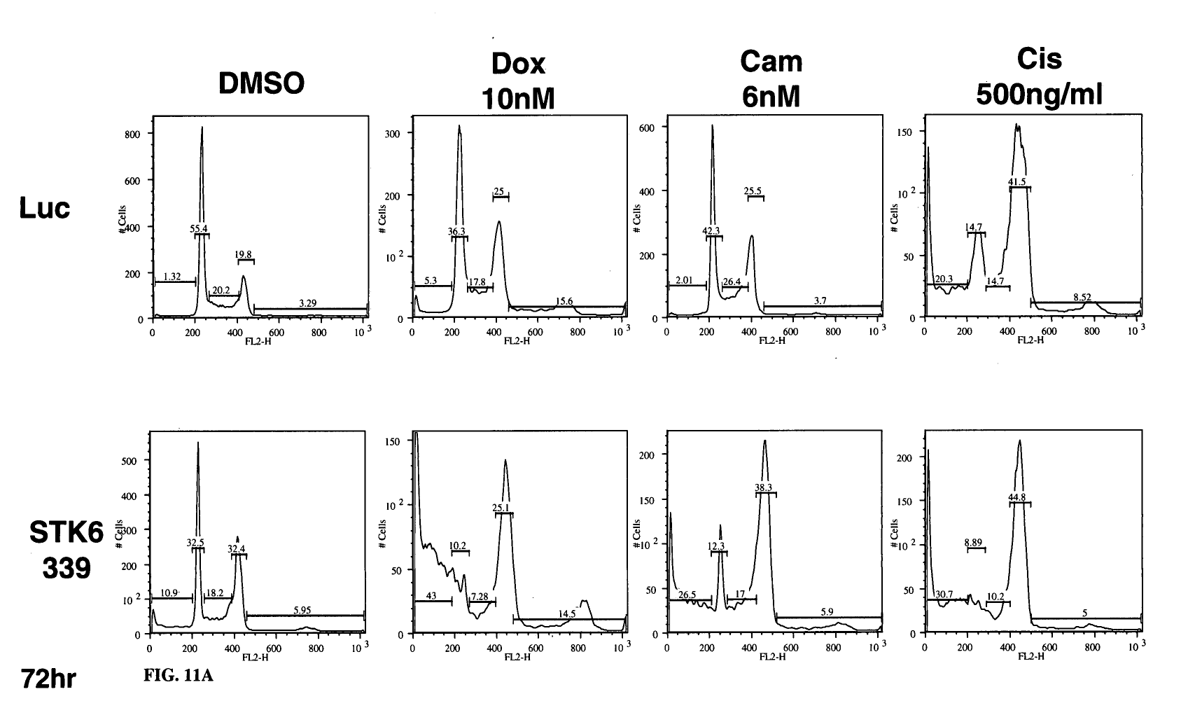

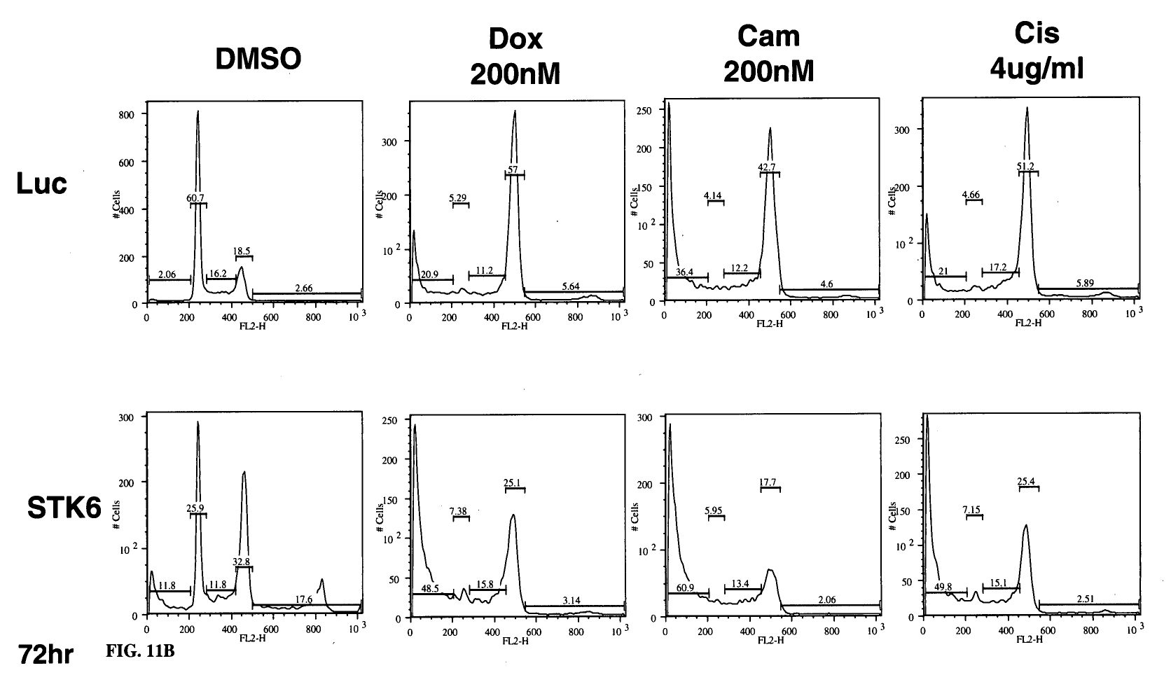

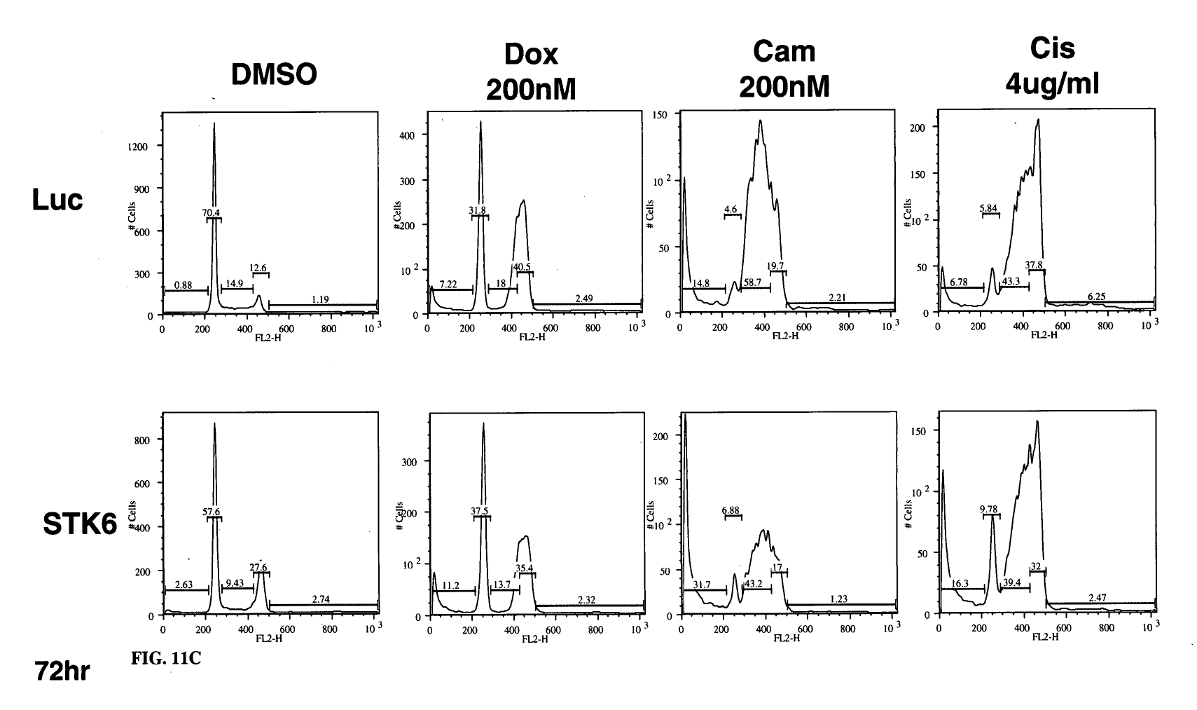

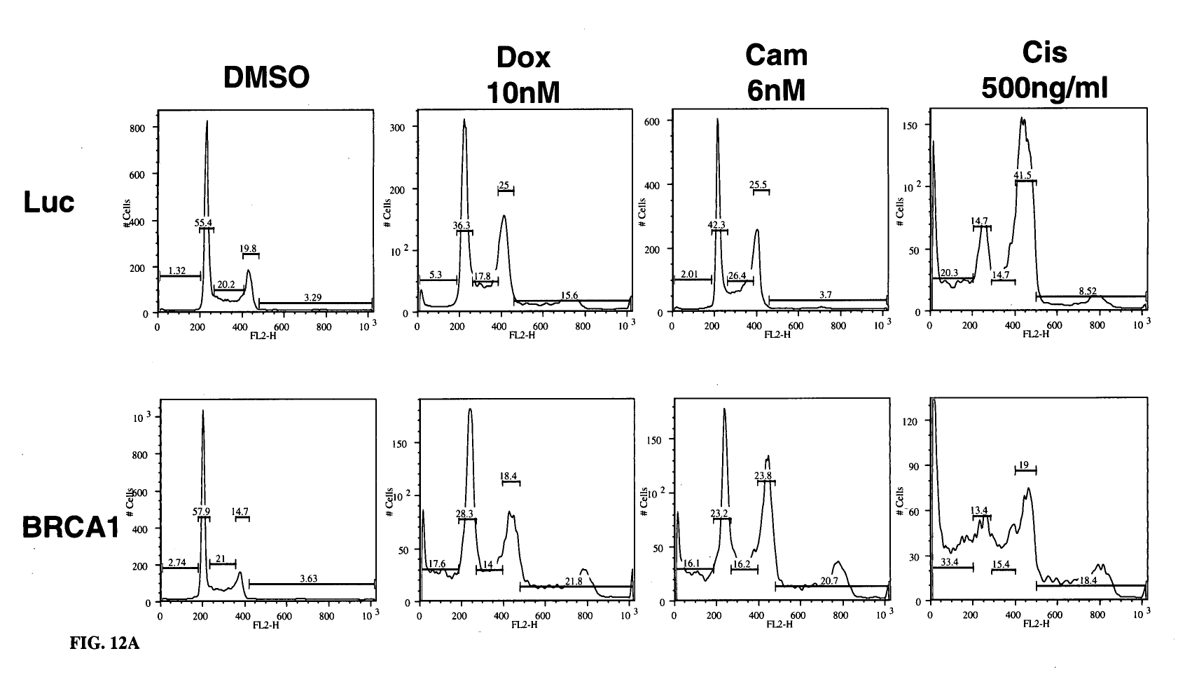

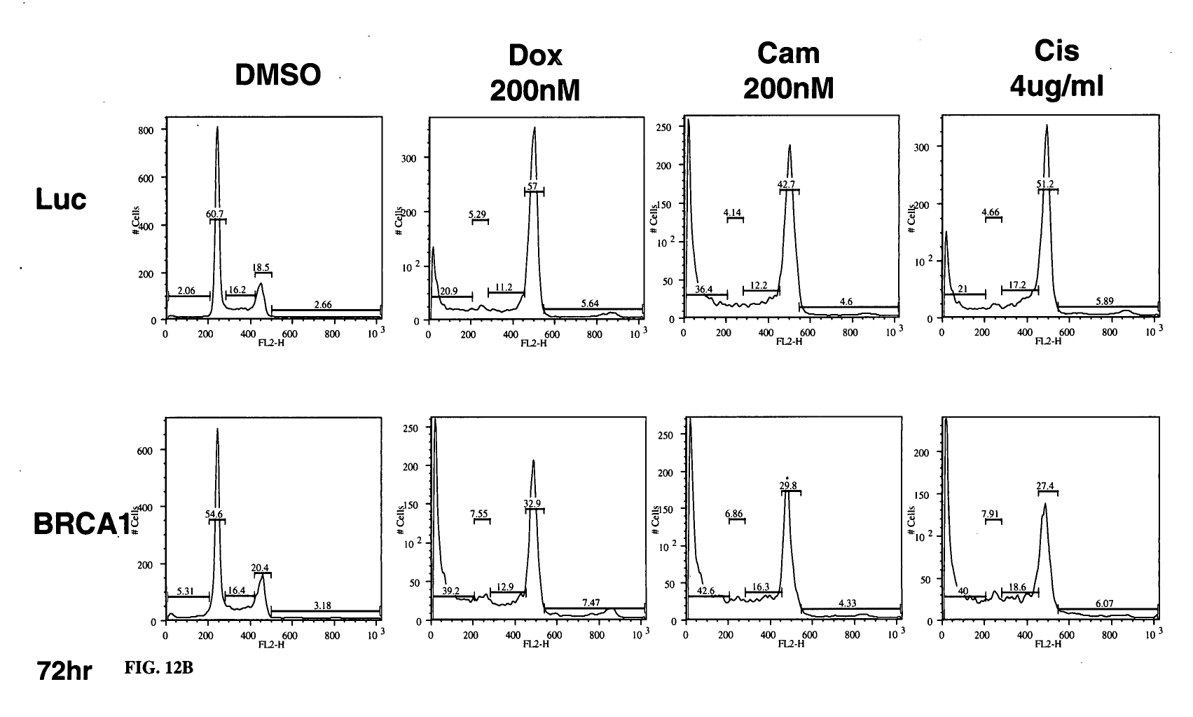

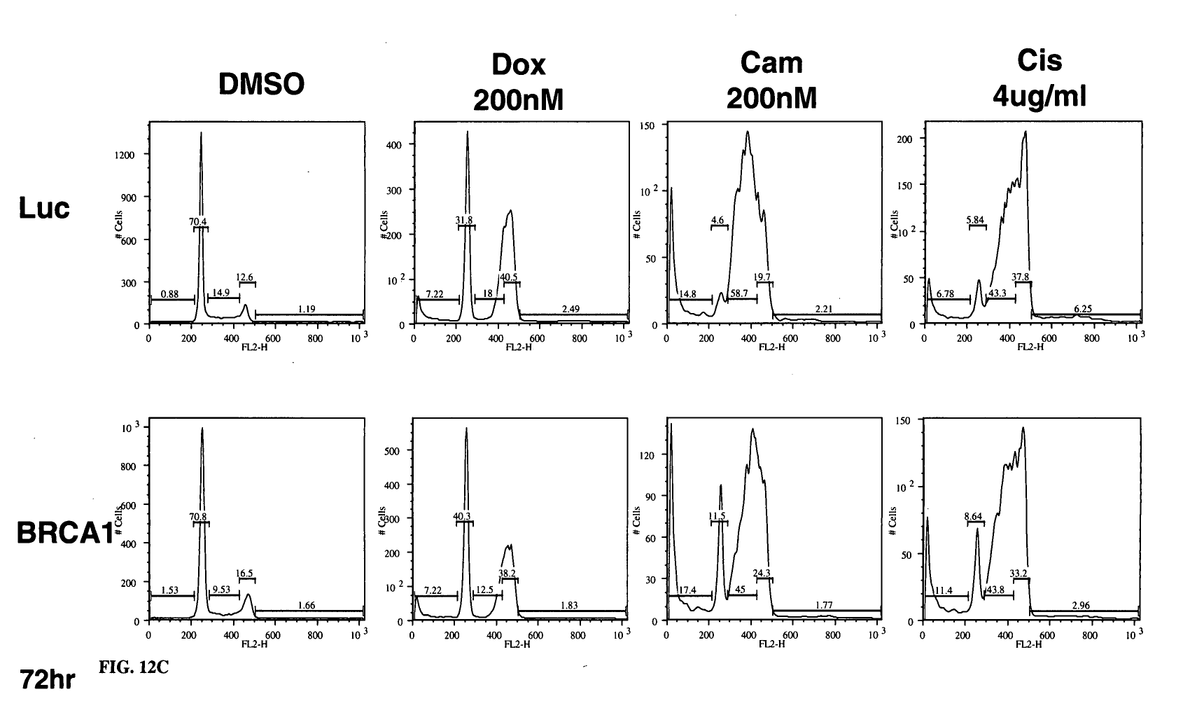

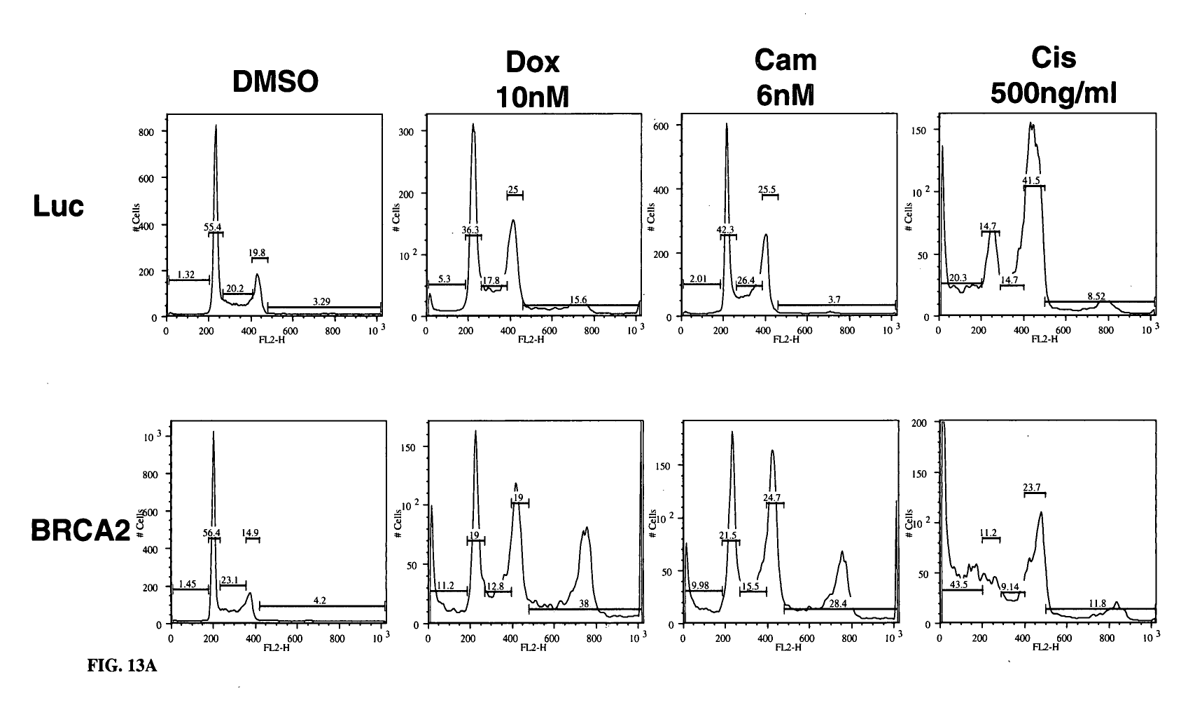

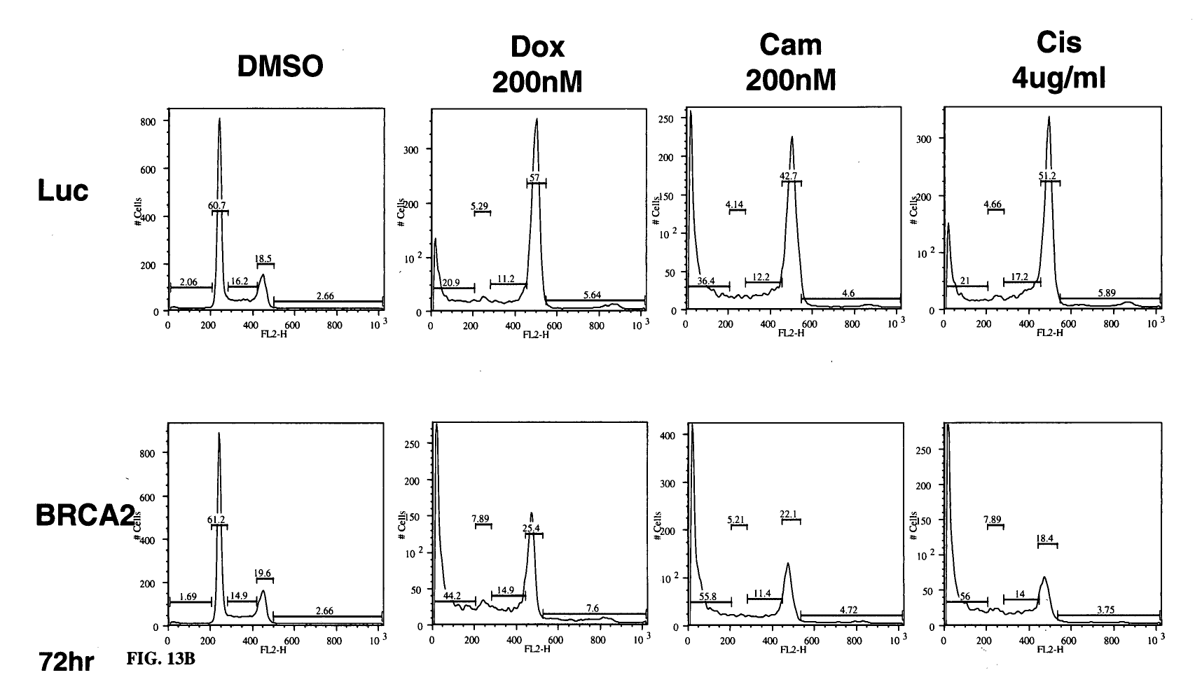

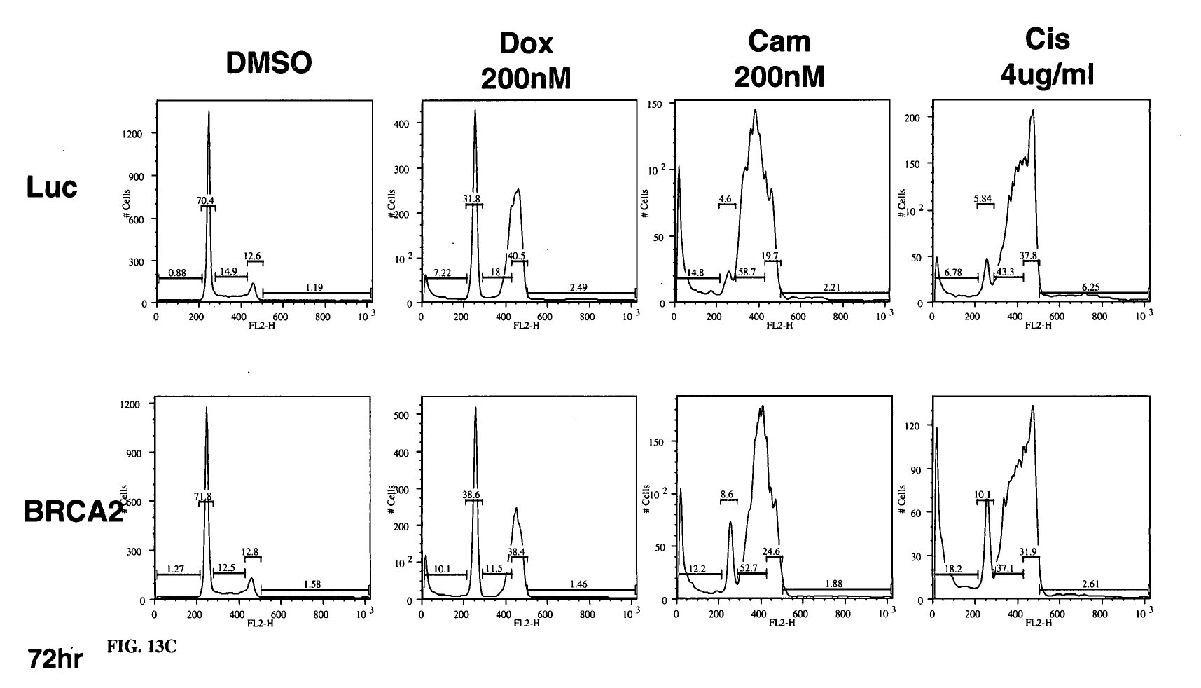

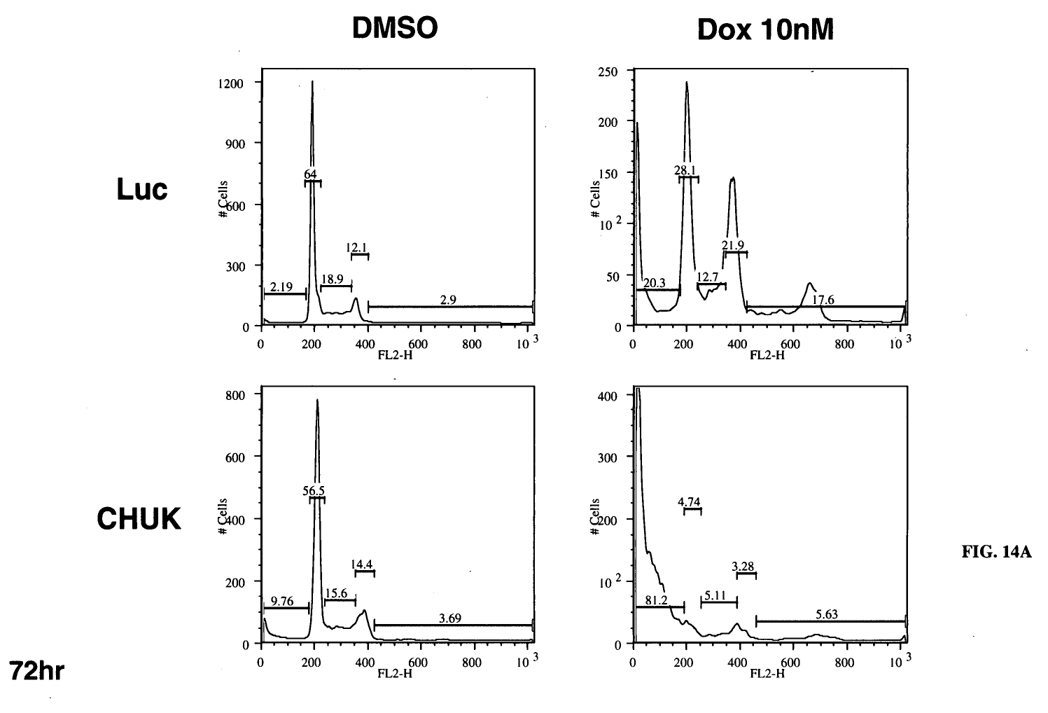

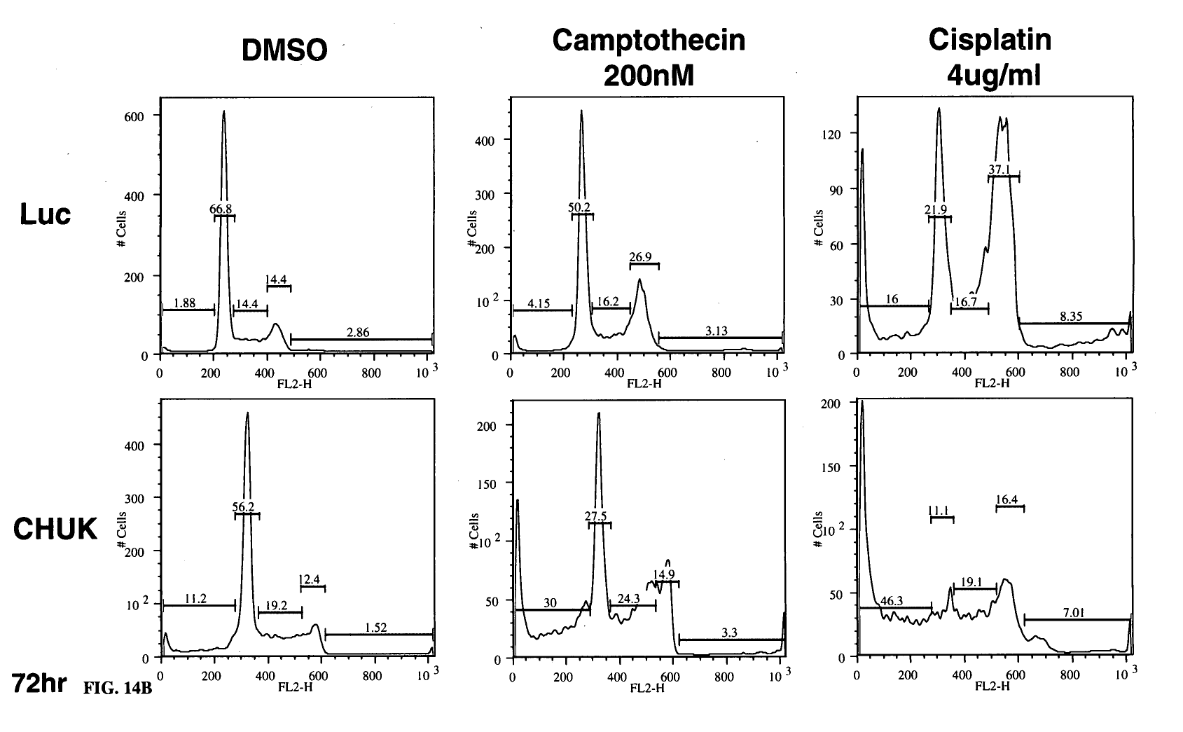

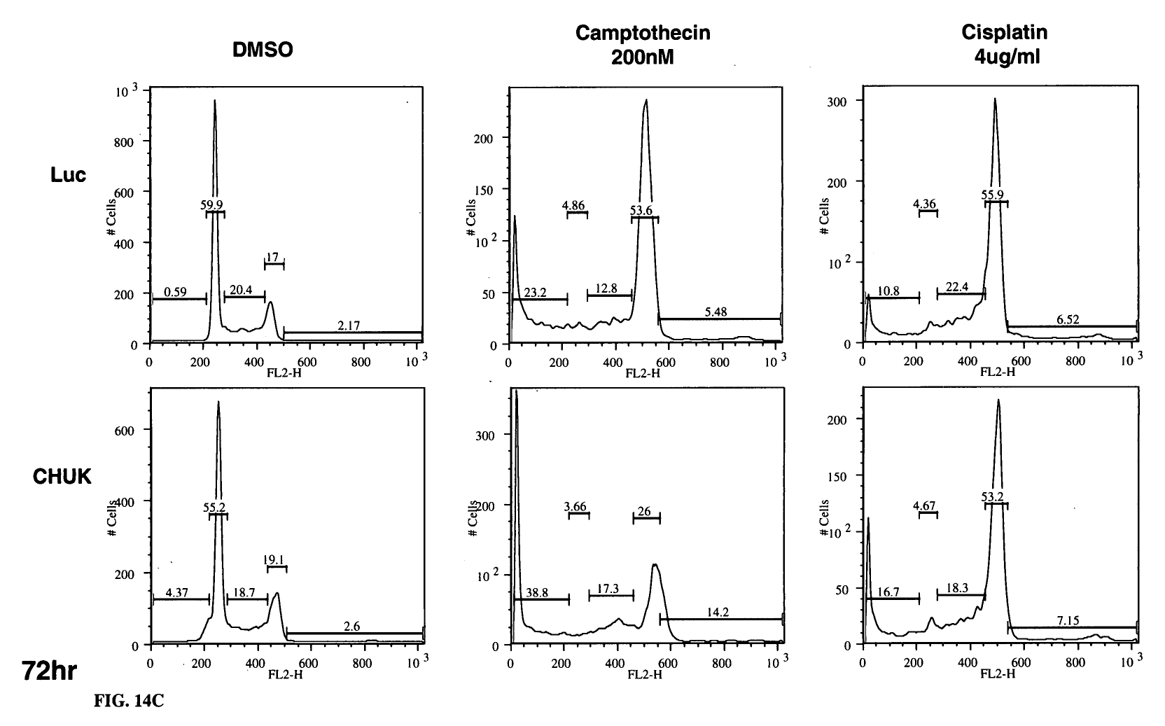

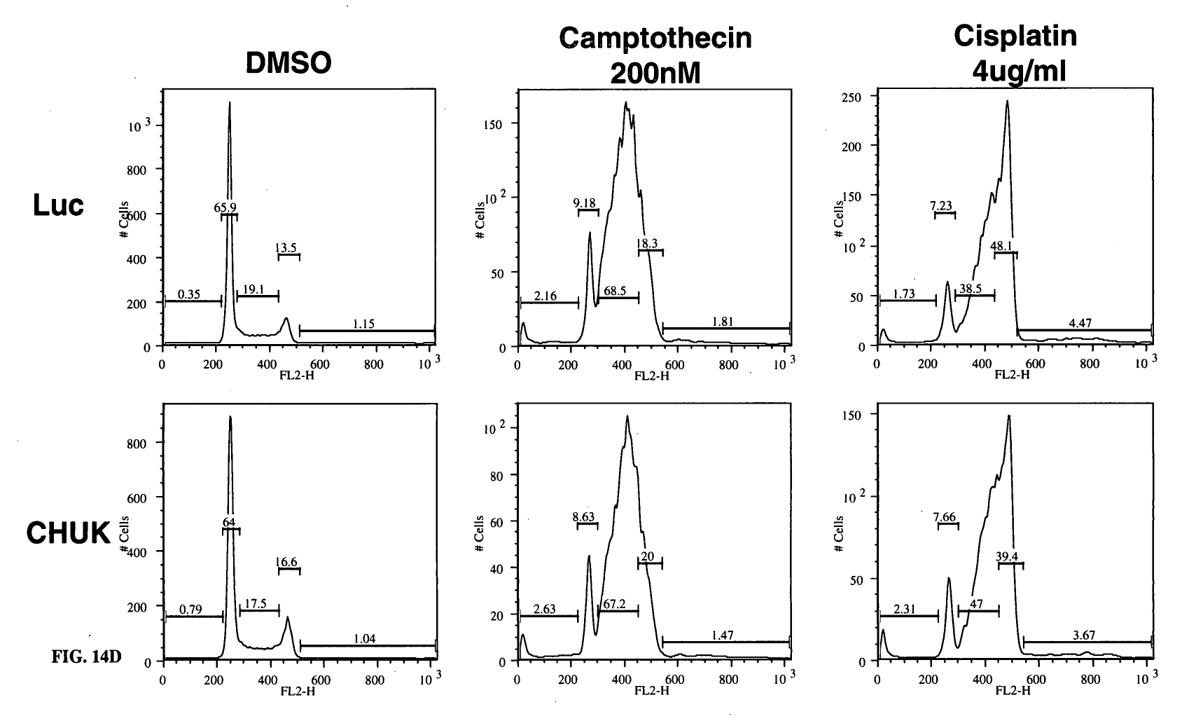

FIGS. 9-14 show the results of the secondary screens. FIGS. 9A-9C show that silencing of WEE1 sensitizes HeLa cells to DNA damage induced by Dox, Campto, and Cis. FIGS. 9D-9I show that silencing of WEE1 sensitizes p53− A549 cells to DNA damage induced by Dox, Campto, and Cis, but does not sensitize p53+ A549 cells to such DNA damage. FIGS. 10A-10C show that silencing of EPHB3 sensitizes HeLa cells and p53− A549 C7, and to a lesser extent p53+ A549 pRS cells, to DNA damage induced by Dox, Campto, and Cis. FIGS. 11A-11C show that silencing of STK6 sensitizes HeLa cells and p53− A549 C7, and to a lesser extent p53+ A549 pRS cells to DNA damage induced by Dox, Campto, and Cis. FIGS. 12A-12C show that silencing of BRCA1 sensitizes HeLa cells and p53− A549 C7 cells to DNA damage induced by Dox, Campto, and Cis. Silencing of BRCA also sensitizes p53+ A549 pRS cells to DNA damage induced by Cis to a lesser extent, but does not sensitize p53+ A549 pRS cells to DNA damage induced by Dox and Campto. FIGS. 13A-13B show that silencing of BRCA2 sensitizes HeLa cells and p53− A549 C7 cells to DNA damage induced by Dox, Campto, and Cis. FIG. 13C shows that silencing of BRCA2 sensitize p53+ A549 pRS cells to DNA damage induced by Cis to a lesser extent, but not dox and Campto. FIGS. 14A-14B show that silencing of CHUK sensitizes HeLa cells to DNA damage induced by Dox, Campto, and Cis. FIG. 14C shows that silencing of CHUK sensitizes p53− A549 C7 cells to DNA damage induced by Campto, and Cis. FIG. 14D shows that silencing of CHUK does not sensitize p53+ A549 pRS cells to DNA damage induced by Campto and Cis.

TABLE IIA
Average fold sensitization by cisplatin

| | | | ave fold | |
|---|---|---|---|---|
| Gene ID | | Gene Name | sensitization | std dev |
| 1 | 2514 | PLK | 0.302987553 | 0.122442 |
| 2 | 3099 | PLK | 0.344442634 | 0.157221 |
| 3 | 3433 | PLK | 0.415618617 | 0.142888 |
| 4 | 3266 | PLK | 0.471258534 | 0.273419 |
| 5 | 3006 | PLK | 0.573026377 | 0.295022 |
| 6 | 3534 | PLK | 0.580135373 | 0.403069 |
| 7 | 3806 | C10orf3 | 0.581678284 | 0.122098 |
| 8 | 3322 | CCNA2 | 0.603615299 | 0.027899 |
| 9 | 3805 | C20orf1 | 0.618083836 | 0.081029 |
| 10 | 3423 | NM_006101 | 0.640054878 | 0.131981 |
| 11 | 3464 | INSR | 0.67184541 | 0.043498 |
| 12 | 3722 | TLK2 | 0.680201667 | 0.164793 |
| 13 | 3731 | CSNK1E | 0.70971928 | 0.169767 |
| 14 | 3261 | ERBB2 | 0.721804997 | 0.095466 |
| 15 | 3093 | PIK3CG | 0.730517635 | 0.16341 |
| 16 | 3391 | PLK | 0.73566872 | 0.438713 |
| 17 | 3813 | ANLN | 0.742286686 | 0.076826 |
| 18 | 3687 | CAMK4 | 0.763785182 | 0.078326 |
| 19 | 3838 | PRKAA2 | 0.768128477 | 0.098461 |
| 20 | 2702 | 2702 | 0.77422078 | 0.032982 |
| 21 | 3435 | FLT3 | 0.786069641 | 0.033061 |
| 22 | 3740 | STK35 | 0.786251834 | 0.241352 |
| 23 | 3826 | NM_015694 | 0.78668619 | 0.158833 |
| 24 | 3113 | CNK | 0.789751097 | 0.074976 |
| 25 | 3648 | CLK1 | 0.795962486 | 0.119858 |
| 26 | 3397 | BUB3 | 0.798897309 | 0.041819 |
| 27 | 2982 | CDC2L2 | 0.803290264 | 0.28261 |
| 28 | 2975 | NEK4 | 0.804972926 | 0.092313 |
| 29 | 3003 | PER | 0.806761229 | 0.283308 |
| 30 | 3776 | NOTCH2 | 0.807626974 | 0.090463 |
| 31 | 3600 | RRM2B | 0.807791139 | 0.116058 |
| 32 | 3303 | CDKN2D | 0.808236038 | 0.106543 |
| 33 | 3536 | PIK3C3 | 0.811623871 | 0.072924 |
| 34 | 3491 | PRKCE | 0.818554314 | 0.081903 |
| 35 | 3181 | ST5 | 0.820227877 | 0.105561 |
| 36 | 3812 | CDCA8 | 0.825194175 | 0.149709 |
| 37 | 3525 | NOTCH4 | 0.826075824 | 0.135465 |
| 38 | 3182 | MYCN | 0.826997754 | 0.074996 |
| 39 | 2992 | PRKR | 0.83026411 | 0.107682 |
| 40 | 2972 | KSR | 0.840737073 | 0.220722 |
| 41 | 3359 | TUBA1 | 0.841656288 | 0.176344 |
| 42 | 3183 | NM_005200 | 0.843755002 | 0.126232 |
| 43 | 2961 | PIM1 | 0.846814316 | 0.1791 |
| 44 | 3814 | HMMR | 0.848584565 | 0.089675 |
| 45 | 3326 | CCT7 | 0.850805908 | 0.139648 |
| 46 | 3819 | TACC3 | 0.851051224 | 0.151449 |
| 47 | 3495 | FGFR2 | 0.851658058 | 0.169414 |
| 48 | 2952 | PRKG1 | 0.853083744 | 0.103483 |
| 49 | 3680 | CLK3 | 0.853111421 | 0.029348 |
| 50 | 3650 | NM_025195 | 0.855769333 | 0.097938 |
| 51 | 3635 | STAT1 | 0.856732819 | 0.045221 |
| 52 | 3487 | MAP2K3 | 0.858609643 | 0.046727 |
| 53 | 3831 | CLSPN | 0.865300447 | 0.043122 |
| 54 | 3416 | IKBKE | 0.868770694 | 0.033925 |
| 55 | 3693 | NEK9 | 0.871865115 | 0.272749 |
| 56 | 3686 | MAP3K8 | 0.872321606 | 0.276021 |
| 57 | 3677 | HCK | 0.874242862 | 0.099478 |
| 58 | 3509 | KIF21A | 0.876152348 | 0.070276 |
| 59 | 3666 | PAK6 | 0.877347139 | 0.070142 |
| 60 | 3563 | RAB3A | 0.877392452 | 0.07511 |
| 61 | 2993 | SRMS | 0.877914429 | 0.052743 |
| 62 | 3658 | STK18 | 0.884409716 | 0.022945 |
| 63 | 3153 | RB1 | 0.884802012 | 0.066909 |
| 64 | 3000 | BMX | 0.88790935 | 0.05788 |
| 65 | 3784 | MAPK8 | 0.888444434 | 0.124134 |
| 66 | 3503 | EGR1 | 0.8888158 | 0.172111 |
| 67 | 3578 | RREB1 | 0.889406356 | 0.126074 |
| 68 | 3085 | KIF5C | 0.889747874 | 0.062749 |
| 69 | 3431 | NM_018454 | 0.893082893 | 0.124062 |
| 70 | 2954 | ROCK2 | 0.893933798 | 0.055935 |
| 71 | 2922 | NM_004783 | 0.89487587 | 0.052019 |
| 72 | 3631 | WISP2 | 0.895799222 | 0.04132 |
| 73 | 3752 | CCNB3 | 0.895903064 | 0.014712 |
| 74 | 3808 | CKAP2 | 0.897429532 | 0.077036 |
| 75 | 3399 | HSPCB | 0.898588123 | 0.283379 |
| 76 | 3251 | ABL1 | 0.899747173 | 0.09061 |
| 77 | 3695 | PRKAA1 | 0.899926191 | 0.099839 |
| 78 | 3319 | CCND1 | 0.901342596 | 0.14162 |
| 79 | 3786 | FRAP1 | 0.901481586 | 0.064389 |
| 80 | 2964 | RIPK2 | 0.901658094 | 0.057156 |
| 81 | 3179 | PDGFB | 0.902358454 | 0.054703 |
| 82 | 2987 | RNASEL | 0.90485908 | 0.109916 |
| 83 | 3086 | KIF11 | 0.905925473 | 0.044166 |
| 84 | 3610 | LEF1 | 0.906026445 | 0.269465 |
| 85 | 3798 | ACTR2 | 0.9086166 | 0.162743 |
| 86 | 3088 | KIF13B | 0.912159346 | 0.09222 |
| 87 | 3332 | CDC5L | 0.912625936 | 0.141471 |
| 88 | 3711 | LIMK1 | 0.912891621 | 0.150911 |
| 89 | 3775 | NOTCH1 | 0.914649314 | 0.049686 |
| 90 | 3743 | RAGE | 0.915875434 | 0.062887 |
| 91 | 3410 | RPS27 | 0.916611322 | 0.14842 |
| 92 | 3403 | AURKC | 0.917162845 | 0.112884 |
| 93 | 3197 | ARHB | 0.917549671 | 0.07581 |
| 94 | 3145 | C20orf23 | 0.918517448 | 0.040236 |
| 95 | 2980 | RIPK1 | 0.918693241 | 0.035801 |
| 96 | 3646 | NM_005781 | 0.919629184 | 0.074213 |
| 97 | 3256 | CDC2L1 | 0.920311861 | 0.161437 |
| 98 | 3171 | VHL | 0.921197139 | 0.154964 |
| 99 | 3661 | FGR | 0.921903863 | 0.062718 |
| 100 | 2978 | AB067470 | 0.922713135 | 0.058126 |
| 101 | 2983 | GUCY2C | 0.922891001 | 0.132499 |
| 102 | 3557 | JUND | 0.923386231 | 0.212516 |
| 103 | 3573 | NM_016848 | 0.924255509 | 0.025747 |
| 104 | 3783 | KRAS2 | 0.924335869 | 0.031975 |
| 105 | 3833 | ATR | 0.925151796 | 0.036459 |
| 106 | 3762 | MCC | 0.926766797 | 0.063215 |
| 107 | 2934 | IRAK2 | 0.927137542 | 0.090048 |
| 108 | 3311 | CDK10 | 0.927487493 | 0.197303 |
| 109 | 3230 | MAP2K1 | 0.929528292 | 0.087866 |
| 110 | 3461 | KIT | 0.929864607 | 0.065105 |
| 111 | 3581 | RASGRP1 | 0.930046334 | 0.085936 |
| 112 | 3782 | SOS1 | 0.93078276 | 0.086957 |
| 113 | 3348 | DCK | 0.932934579 | 0.140927 |
| 114 | 3518 | NFKB1 | 0.933538042 | 0.254776 |
| 115 | 3692 | AB007941 | 0.934031479 | 0.122891 |
| 116 | 2936 | SGKL | 0.935268856 | 0.12869 |
| 117 | 3788 | PRKCE | 0.935825459 | 0.100437 |
| 118 | 3791 | NM_005200 | 0.937373151 | 0.124551 |
| 119 | 3827 | NM_018123 | 0.938752687 | 0.120885 |
| 120 | 3343 | CENPJ | 0.939276361 | 0.15064 |
| 121 | 3413 | KIF23 | 0.940719223 | 0.224476 |
| 122 | 3540 | PPP2CB | 0.940825549 | 0.07786 |
| 123 | 3559 | RAP1GDS1 | 0.941186098 | 0.092318 |
| 124 | 2943 | DYRK2 | 0.941751587 | 0.079768 |
| 125 | 3090 | KIF3C | 0.942994713 | 0.043187 |
| 126 | 3306 | CDC14A | 0.943159212 | 0.105314 |
| 127 | 3572 | RASA3 | 0.943756386 | 0.044924 |
| 128 | 3822 | GTSE1 | 0.944755556 | 0.2332 |
| 129 | 3351 | ESR1 | 0.944920378 | 0.153622 |
| 130 | 3258 | MOS | 0.9460337 | 0.090205 |
| 131 | 3601 | POLE | 0.94708241 | 0.126731 |
| 132 | 2960 | LYN | 0.947322877 | 0.19148 |
| 133 | 3828 | KIF20A | 0.950773558 | 0.183938 |
| 134 | 3778 | VHL | 0.951938861 | 0.232481 |
| 135 | 3196 | ARHI | 0.952842248 | 0.058681 |
| 136 | 3566 | JUN | 0.95294025 | 0.127285 |
| 137 | 3240 | MAPK12 | 0.953564717 | 0.071586 |
| 138 | 3184 | TSG101 | 0.954002138 | 0.04823 |
| 139 | 3714 | NM_013355 | 0.954197885 | 0.12488 |
| 140 | 3364 | HPRT1 | 0.954414394 | 0.271771 |
| 141 | 3685 | LTK | 0.954443302 | 0.285398 |
| 142 | 3751 | BCR | 0.954467451 | 0.121004 |
| 143 | 3434 | DDX6 | 0.954790843 | 0.082973 |
| 144 | 3298 | CCNE1 | 0.955113281 | 0.080149 |
| 145 | 3449 | TBK1 | 0.955301632 | 0.018969 |
| 146 | 3795 | NR4A2 | 0.955557277 | 0.096686 |
| 147 | 3739 | NM_017886 | 0.955581637 | 0.103771 |
| 148 | 3471 | MAPK10 | 0.956705519 | 0.068765 |
| 149 | 3139 | XM_095827 | 0.956993628 | 0.217327 |
| 150 | 3545 | IRS2 | 0.957861116 | 0.058638 |
| 151 | 2985 | MKNK1 | 0.958784274 | 0.02755 |
| 152 | 3618 | DVL2 | 0.958860428 | 0.145917 |
| 153 | 3726 | MAPKAPK2 | 0.95922853 | 0.071282 |
| 154 | 3678 | PFTK1 | 0.960709464 | 0.043435 |
| 155 | 3821 | ASPM | 0.961220945 | 0.129044 |
| 156 | 3163 | THRA | 0.96204376 | 0.138031 |
| 157 | 3101 | MAPK14 | 0.962194967 | 0.089772 |
| 158 | 3561 | FOS | 0.96220097 | 0.038394 |
| 159 | 3133 | XM_168069 | 0.962355545 | 0.075119 |
| 160 | 3443 | EPS8 | 0.962670509 | 0.080284 |
| 161 | 3117 | ATM | 0.963448158 | 0.17684 |
| 162 | 3401 | HDAC1 | 0.963594921 | 0.053087 |
| 163 | 3799 | ACTR3 | 0.96385153 | 0.106281 |
| 164 | 3733 | MYLK2 | 0.96390586 | 0.071956 |
| 165 | 3801 | PSEN1 | 0.96432309 | 0.133399 |
| 166 | 3716 | ULK1 | 0.964714374 | 0.172756 |
| 167 | 2977 | RIPK3 | 0.965321488 | 0.288006 |
| 168 | 3571 | VAV1 | 0.966085791 | 0.040696 |
| 169 | 2946 | NM_017719 | 0.966726854 | 0.070416 |
| 170 | 3459 | EGFR | 0.968475197 | 0.03989 |
| 171 | 3835 | CHEK2 | 0.968492479 | 0.077394 |
| 172 | 3125 | NM_031217 | 0.968705878 | 0.158815 |
| 173 | 3308 | CDKN2B | 0.970454697 | 0.030316 |
| 174 | 3458 | ARAF1 | 0.972126514 | 0.150383 |
| 175 | 3162 | MADH2 | 0.97228749 | 0.077251 |
| 176 | 2949 | MYO3B | 0.973636618 | 0.06916 |
| 177 | 3664 | STK17A | 0.975343312 | 0.06811 |
| 178 | 3488 | AURKB | 0.975742132 | 0.178321 |
| 179 | 3112 | KNSL7 | 0.976665103 | 0.157911 |
| 180 | 3485 | DHX8 | 0.978053596 | 0.073262 |
| 181 | 3809 | CDCA3 | 0.979002265 | 0.231181 |
| 182 | 3161 | WT1 | 0.979221693 | 0.114838 |
| 183 | 3513 | ROS1 | 0.979271577 | 0.121589 |
| 184 | 3185 | VCAM1 | 0.979438257 | 0.069759 |
| 185 | 3553 | CKS1B | 0.979465469 | 0.05555 |
| 186 | 3763 | NM_016231 | 0.980990574 | 0.123555 |
| 187 | 3245 | AXL | 0.981022783 | 0.078724 |
| 188 | 3334 | CUL4B | 0.981485893 | 0.048462 |
| 189 | 3193 | FGF3 | 0.981515057 | 0.075982 |
| 190 | 3335 | CDK5R2 | 0.983188137 | 0.095535 |
| 191 | 3455 | MAP2K4 | 0.984383299 | 0.095921 |
| 192 | 2925 | FYN | 0.984597535 | 0.177611 |
| 193 | 3215 | MAD2L1 | 0.984674289 | 0.166817 |
| 194 | 3519 | NTRK1 | 0.985625526 | 0.225903 |
| 195 | 2541 | 2541 | 0.985658529 | 0.02934 |
| 196 | 3109 | KIF1C | 0.985891536 | 0.162583 |
| 197 | 3792 | ARHGEF1 | 0.985986394 | 0.150503 |
| 198 | 3374 | POLR2A | 0.986875398 | 0.174675 |
| 199 | 3362 | NR3C1 | 0.98711375 | 0.09249 |
| 200 | 3231 | ILK | 0.987500124 | 0.068942 |
| 201 | 3166 | PMS1 | 0.987593476 | 0.040016 |
| 202 | 3703 | AK024504 | 0.988238947 | 0.078314 |
| 203 | 3707 | TXK | 0.98900485 | 0.138186 |
| 204 | 3323 | CDK5R1 | 0.989595604 | 0.176376 |
| 205 | 3180 | CD44 | 0.990121058 | 0.090413 |
| 206 | 3630 | WISP3 | 0.990225627 | 0.071631 |
| 207 | 3576 | GRAP | 0.990505346 | 0.120959 |
| 208 | 3800 | CHFR | 0.99369692 | 0.117429 |
| 209 | 3142 | KIF25 | 0.993932342 | 0.044087 |
| 210 | 3160 | TACSTD1 | 0.994447265 | 0.128513 |
| 211 | 3497 | EPHA8 | 0.99446771 | 0.015206 |
| 212 | 3757 | CLK4 | 0.995683284 | 0.166859 |
| 213 | 3645 | CASK | 0.996395727 | 0.07959 |
| 214 | 3357 | PRIM2A | 0.998092371 | 0.117247 |
| 215 | 3594 | RAP2A | 0.99814842 | 0.142818 |
| 216 | 3796 | ARHGEF6 | 0.998577367 | 0.091413 |
| 217 | 3767 | FZD3 | 0.99921132 | 0.096395 |
| 218 | 3715 | CDC42BPA | 0.999524848 | 0.196389 |
| 219 | 2938 | ALS2CR7 | 0.99966653 | 0.007718 |
| 220 | 3419 | RFC4 | 0.999756476 | 0.079342 |
| 221 | 63 | M15077 | 1 | 0 |
| 222 | 3672 | SYK | 1.000094306 | 0.028316 |
| 223 | 3832 | ATM | 1.00019002 | 0.091546 |
| 224 | 3627 | CTNNA1 | 1.000291459 | 0.215453 |
| 225 | 2984 | EPHB6 | 1.000603948 | 0.098044 |
| 226 | 3200 | REL | 1.000616585 | 0.104464 |
| 227 | 3492 | PRKCQ | 1.000785085 | 0.103181 |
| 228 | 3478 | EPHA2 | 1.001756995 | 0.101444 |
| 229 | 3539 | PLCG2 | 1.002008086 | 0.072305 |
| 230 | 3378 | NM_006009 | 1.002912861 | 0.019886 |
| 231 | 3381 | POLR2B | 1.003653073 | 0.021542 |
| 232 | 3452 | JAK1 | 1.00410017 | 0.246916 |
| 233 | 2926 | AF172264 | 1.0043601 | 0.195291 |
| 234 | 3641 | TYRO3 | 1.005062954 | 0.13144 |
| 235 | 3750 | CAMK2A | 1.005879519 | 0.197981 |
| 236 | 3595 | FEN1 | 1.006107713 | 0.1559 |
| 237 | 3375 | AHCY | 1.006847357 | 0.098914 |
| 238 | 3367 | DHFR | 1.007697409 | 0.048476 |
| 239 | 3555 | RASA1 | 1.007907357 | 0.060107 |
| 240 | 3246 | RPS6KB1 | 1.008295705 | 0.098199 |
| 241 | 3551 | STAT3 | 1.008697776 | 0.069559 |
| 242 | 3708 | RPS6KC1 | 1.008738004 | 0.158539 |
| 243 | 3820 | NM_018410 | 1.008803441 | 0.00423 |
| 244 | 3548 | RAC1 | 1.009000664 | 0.10905 |
| 245 | 3527 | DTX2 | 1.00940399 | 0.082767 |
| 246 | 3339 | CCNB2 | 1.009625325 | 0.321434 |
| 247 | 3226 | RBX1 | 1.01029159 | 0.235969 |
| 248 | 3473 | DAPK1 | 1.010335394 | 0.065266 |
| 249 | 3469 | AAK1 | 1.011653085 | 0.153819 |
| 250 | 3517 | MYC | 1.011855757 | 0.088032 |
| 251 | 3005 | MERTK | 1.011910266 | 0.139112 |
| 252 | 3294 | CCNF | 1.01355402 | 0.151217 |
| 253 | 3392 | BIRC5 | 1.014018575 | 0.147292 |
| 254 | 3533 | HES7 | 1.016868954 | 0.209244 |
| 255 | 3524 | NOTCH3 | 1.017285472 | 0.068877 |
| 256 | 3587 | VAV3 | 1.018129173 | 0.062737 |
| 257 | 3425 | DLG7 | 1.018264827 | 0.037325 |
| 258 | 3674 | CSNK1D | 1.018650699 | 0.087521 |
| 259 | 3380 | TUBG2 | 1.019248432 | 0.027697 |
| 260 | 3486 | RPS6KA3 | 1.019985527 | 0.050031 |
| 261 | 3746 | HUNK | 1.020779918 | 0.082372 |
| 262 | 3535 | SKP2 | 1.021142953 | 0.100064 |
| 263 | 3797 | ARHGEF9 | 1.021635562 | 0.137783 |
| 264 | 2969 | NM_014916 | 1.021887811 | 0.080467 |
| 265 | 3460 | CSK | 1.022085366 | 0.135805 |
| 266 | 3132 | KIF23 | 1.023806782 | 0.129496 |
| 267 | 2963 | MAP3K11 | 1.0242873 | 0.065223 |
| 268 | 3702 | MAP3K13 | 1.024294874 | 0.083014 |
| 269 | 3382 | TUBB | 1.025915608 | 0.049937 |
| 270 | 3237 | CDC7 | 1.025994603 | 0.096409 |
| 271 | 3592 | SOS2 | 1.026235513 | 0.178995 |
| 272 | 3365 | PRIM1 | 1.02653855 | 0.104798 |
| 273 | 3570 | RALGDS | 1.027460697 | 0.084873 |
| 274 | 3224 | FBXO5 | 1.029155584 | 0.154545 |
| 275 | 3585 | GAB1 | 1.029481526 | 0.06077 |
| 276 | 3414 | HDAC7A | 1.030424895 | 0.139587 |
| 277 | 3514 | HRAS | 1.030481671 | 0.09281 |
| 278 | 3597 | SHMT2 | 1.031207997 | 0.180827 |
| 279 | 3657 | PCTK1 | 1.031839128 | 0.067828 |
| 280 | 3257 | IGF1R | 1.03192729 | 0.10264 |
| 281 | 3773 | WNT2 | 1.032309731 | 0.174004 |
| 282 | 3625 | CTBP2 | 1.032538009 | 0.159078 |
| 283 | 3302 | CDK8 | 1.032760545 | 0.077771 |
| 284 | 3409 | TTK | 1.033113517 | 0.089383 |
| 285 | 3465 | EPHA1 | 1.033516487 | 0.127809 |
| 286 | 3705 | NM_012119 | 1.033751364 | 0.107948 |
| 287 | 2966 | NM_033266 | 1.035012335 | 0.097641 |
| 288 | 2999 | FES | 1.035558582 | 0.12725 |
| 289 | 3474 | CSNK2A1 | 1.036151218 | 0.085057 |
| 290 | 3824 | MAPRE3 | 1.036197838 | 0.092706 |
| 291 | 3094 | KIF3A | 1.036464942 | 0.119921 |
| 292 | 3769 | PLAU | 1.037390211 | 0.064893 |
| 293 | 3213 | NM_016238 | 1.037745909 | 0.138786 |
| 294 | 2950 | NEK6 | 1.038291854 | 0.149776 |
| 295 | 3815 | MAPRE2 | 1.038305947 | 0.217709 |
| 296 | 3543 | PDK2 | 1.038311221 | 0.197679 |
| 297 | 3437 | FGFR1 | 1.0383269 | 0.283643 |
| 298 | 3542 | PPP2CA | 1.039357671 | 0.194352 |
| 299 | 3511 | XM_168069 | 1.039370913 | 0.155262 |
| 300 | 3002 | CRKL | 1.039983971 | 0.101129 |
| 301 | 3398 | HDAC11 | 1.041663934 | 0.099406 |
| 302 | 3675 | ADRBK1 | 1.041741459 | 0.084419 |
| 303 | 3623 | CTNND1 | 1.042210238 | 0.105978 |
| 304 | 3268 | CDC25C | 1.042762357 | 0.02726 |
| 305 | 3633 | CTBP1 | 1.042818569 | 0.143171 |
| 306 | 3804 | NM_024322 | 1.042895573 | 0.05266 |
| 307 | 3526 | HES6 | 1.043146787 | 0.059244 |
| 308 | 2947 | NM_007064 | 1.043456689 | 0.080305 |
| 309 | 2979 | PAK2 | 1.043537793 | 0.115188 |
| 310 | 2959 | PIM2 | 1.043942064 | 0.050352 |
| 311 | 3602 | MCM3 | 1.044071108 | 0.23865 |
| 312 | 3665 | PAK4 | 1.044246523 | 0.052921 |
| 313 | 3421 | ORC6L | 1.044825423 | 0.241726 |
| 314 | 3745 | CAMKK2 | 1.044966032 | 0.032844 |
| 315 | 3736 | PTK7 | 1.045008777 | 0.118965 |
| 316 | 3119 | CDKN1B | 1.045154749 | 0.026803 |
| 317 | 3643 | DDR2 | 1.045796426 | 0.123748 |
| 318 | 3603 | POLS | 1.046796283 | 0.090212 |
| 319 | 3346 | CCNK | 1.047737442 | 0.148152 |
| 320 | 3438 | DTR | 1.04975054 | 0.139619 |
| 321 | 2942 | TTN | 1.050944386 | 0.134575 |
| 322 | 2937 | NM_025052 | 1.051593448 | 0.052118 |
| 323 | 3577 | RAB2L | 1.051977248 | 0.073992 |
| 324 | 3203 | ITGA5 | 1.052197011 | 0.109443 |
| 325 | 3599 | DTYMK | 1.052206896 | 0.147041 |
| 326 | 3373 | TOP2A | 1.053946926 | 0.061071 |
| 327 | 3222 | PTTG1 | 1.054934465 | 0.059734 |
| 328 | 3154 | MADH4 | 1.055367142 | 0.392285 |
| 329 | 3829 | KIF2C | 1.056017438 | 0.187684 |
| 330 | 3652 | PDGFRA | 1.056020056 | 0.084537 |
| 331 | 2944 | MARK1 | 1.056491568 | 0.161232 |
| 332 | 3656 | PRKCN | 1.056755878 | 0.177943 |
| 333 | 3626 | DVL3 | 1.058711269 | 0.19647 |
| 334 | 3802 | NOTCH3 | 1.059031918 | 0.117495 |
| 335 | 3127 | MAPK1 | 1.059441261 | 0.070449 |
| 336 | 3549 | PIK3R2 | 1.059495493 | 0.178697 |
| 337 | 2935 | MAPK6 | 1.060533709 | 0.075031 |
| 338 | 3307 | CDC6 | 1.060858236 | 0.093933 |
| 339 | 3260 | STK11 | 1.061848445 | 0.120762 |
| 340 | 3766 | S100A2 | 1.062832073 | 0.174576 |
| 341 | 3457 | BAD | 1.063944791 | 0.07637 |
| 342 | 3347 | TOP1 | 1.06614481 | 0.169748 |
| 343 | 3450 | MAP3K2 | 1.066638971 | 0.166869 |
| 344 | 3164 | MYCL1 | 1.066666964 | 0.198532 |
| 345 | 3412 | KIF25 | 1.068536113 | 0.202887 |
| 346 | 3317 | CCNI | 1.068966464 | 0.126188 |
| 347 | 3550 | PLCG1 | 1.069052894 | 0.123064 |
| 348 | 3668 | DAPK3 | 1.069120278 | 0.16697 |
| 349 | 3454 | FLT4 | 1.06985122 | 0.129807 |
| 350 | 3394 | HDAC6 | 1.070168765 | 0.050617 |
| 351 | 3122 | ATSV | 1.071291871 | 0.126675 |
| 352 | 3169 | NME1 | 1.071353382 | 0.062921 |
| 353 | 3342 | CCNT1 | 1.07208287 | 0.030624 |
| 354 | 3523 | NOTCH2 | 1.072235801 | 0.096808 |
| 355 | 3591 | RALB | 1.072637191 | 0.131285 |
| 356 | 2970 | AATK | 1.073460682 | 0.079116 |
| 357 | 3593 | VAV2 | 1.073649235 | 0.087372 |
| 358 | 3489 | SRC | 1.074621049 | 0.096347 |
| 359 | 3363 | GART | 1.076380891 | 0.07919 |
| 360 | 3097 | KIF20A | 1.077741628 | 0.065192 |
| 361 | 3494 | MAPK4 | 1.077922895 | 0.072549 |
| 362 | 3114 | PIK3CD | 1.078095752 | 0.118845 |
| 363 | 2976 | NEK7 | 1.078108286 | 0.543136 |
| 364 | 3352 | NR3C2 | 1.078524745 | 0.200714 |
| 365 | 3115 | MDM2 | 1.079408163 | 0.109166 |
| 366 | 3108 | KIF22 | 1.080326814 | 0.089686 |
| 367 | 2973 | NEK1 | 1.080546527 | 0.210334 |
| 368 | 3219 | CENPC1 | 1.080637703 | 0.211586 |
| 369 | 3583 | JUNB | 1.080828682 | 0.061182 |
| 370 | 3476 | PRKCD | 1.081421932 | 0.063705 |
| 371 | 3717 | NTRK2 | 1.08184551 | 0.179359 |
| 372 | 3760 | CDKL5 | 1.082031957 | 0.122857 |
| 373 | 3744 | PRKWNK4 | 1.082821695 | 0.041089 |
| 374 | 3147 | CDKN2A | 1.083174768 | 0.142556 |
| 375 | 3170 | BLM | 1.083396707 | 0.087103 |
| 376 | 3390 | NM_080925 | 1.083814073 | 0.187306 |
| 377 | 3691 | NM_024046 | 1.084007951 | 0.455202 |
| 378 | 3682 | DYRK1A | 1.085383077 | 0.13164 |
| 379 | 3338 | CUL4A | 1.085696166 | 0.113752 |
| 380 | 3445 | BMPR1A | 1.08653048 | 0.217388 |
| 381 | 3639 | STAT6 | 1.087172241 | 0.240711 |
| 382 | 3683 | NM_003138 | 1.087765627 | 0.107482 |
| 383 | 3694 | STK38 | 1.088925769 | 0.15309 |
| 384 | 3228 | CDC27 | 1.089546461 | 0.230438 |
| 385 | 2923 | ERN1 | 1.090052682 | 0.160503 |
| 386 | 3366 | TYMS | 1.090784989 | 0.157841 |
| 387 | 3816 | NM_017769 | 1.090876067 | 0.170619 |
| 388 | 3107 | KIF2 | 1.091300875 | 0.082185 |
| 389 | 3262 | LATS1 | 1.09148938 | 0.058919 |
| 390 | 3188 | PMS2 | 1.092050213 | 0.140727 |
| 391 | 3498 | CSNK1A1 | 1.092706943 | 0.059983 |
| 392 | 3293 | CDC25A | 1.092986402 | 0.099227 |
| 393 | 3721 | ANKRD3 | 1.093127467 | 0.114101 |
| 394 | 3793 | MAPRE1 | 1.093414458 | 0.107517 |
| 395 | 3305 | CDC2L5 | 1.095069991 | 0.058969 |
| 396 | 3647 | YES1 | 1.095220118 | 0.439175 |
| 397 | 3324 | CUL5 | 1.095253758 | 0.109464 |
| 398 | 2965 | NM_014720 | 1.095861428 | 0.295852 |
| 399 | 3300 | CDC14B | 1.095900812 | 0.053276 |
| 400 | 3296 | CDKN2C | 1.096728587 | 0.06043 |
| 401 | 3724 | EPHA7 | 1.096779937 | 0.20814 |
| 402 | 3165 | FGF2 | 1.099204865 | 0.052442 |
| 403 | 2928 | IRAK1 | 1.099544846 | 0.11705 |
| 404 | 3502 | PRKCH | 1.099795802 | 0.076493 |
| 405 | 3728 | TIE | 1.100408042 | 0.059759 |
| 406 | 3424 | EZH2 | 1.100414429 | 0.137994 |
| 407 | 3756 | CDK5RAP2 | 1.10148794 | 0.169172 |
| 408 | 2920 | EIF2AK3 | 1.101874679 | 0.193517 |
| 409 | 3556 | RAP1A | 1.102603353 | 0.216629 |
| 410 | 3214 | CENPF | 1.102666055 | 0.229565 |
| 411 | 3102 | CKS2 | 1.103490084 | 0.276109 |
| 412 | 2974 | NEK11 | 1.103575721 | 0.38662 |
| 413 | 3297 | CCT2 | 1.103974529 | 0.075386 |
| 414 | 3393 | HDAC2 | 1.104472861 | 0.074707 |
| 415 | 3568 | PLD1 | 1.104812311 | 0.043874 |
| 416 | 3470 | RPS6KA1 | 1.104927224 | 0.121509 |
| 417 | 3496 | EIF4EBP2 | 1.105081332 | 0.026061 |
| 418 | 3432 | PRC1 | 1.105087833 | 0.109514 |
| 419 | 3446 | PRKCG | 1.105375817 | 0.11356 |
| 420 | 3512 | TGFBR1 | 1.106970197 | 0.08351 |
| 421 | 3749 | NM_139021 | 1.107176607 | 0.060956 |
| 422 | 3807 | SPAG5 | 1.107200152 | 0.190375 |
| 423 | 3579 | PDZGEF2 | 1.108384492 | 0.106374 |
| 424 | 3422 | SMC4L1 | 1.108967343 | 0.168462 |
| 425 | 3830 | NM_013296 | 1.109620231 | 0.124287 |
| 426 | 3537 | EIF4EBP1 | 1.110833969 | 0.090069 |
| 427 | 3684 | STK38L | 1.110835442 | 0.127517 |
| 428 | 3681 | SRPK1 | 1.11126095 | 0.138319 |
| 429 | 2990 | NM_015112 | 1.111540967 | 0.290052 |
| 430 | 3605 | FZD4 | 1.111605705 | 0.110001 |
| 431 | 3477 | FGFR4 | 1.111898761 | 0.065007 |
| 432 | 3490 | ERBB3 | 1.113605654 | 0.088278 |
| 433 | 3575 | LATS2 | 1.113869957 | 0.121325 |
| 434 | 3755 | CDKL3 | 1.114362934 | 0.239022 |
| 435 | 3205 | NM_139286 | 1.114649942 | 0.243935 |
| 436 | 3105 | BUB1 | 1.114727935 | 0.21132 |
| 437 | 3389 | NM_052963 | 1.114830338 | 0.092164 |
| 438 | 3110 | KIF13A | 1.116195509 | 0.073039 |
| 439 | 3608 | MAP3K7IP1 | 1.117324513 | 0.193266 |
| 440 | 2957 | TYK2 | 1.11788043 | 0.120323 |
| 441 | 2996 | MAPK3 | 1.117972689 | 0.307163 |
| 442 | 3628 | CTNNBL1 | 1.118548429 | 0.092234 |
| 443 | 3624 | CTNNB1 | 1.118609166 | 0.170984 |
| 444 | 3159 | RET | 1.118867767 | 0.029128 |
| 445 | 3120 | PIK3CB | 1.119135316 | 0.222604 |
| 446 | 3742 | RHOK | 1.119296716 | 0.166613 |
| 447 | 3136 | XM_066649 | 1.119463101 | 0.130616 |
| 448 | 3328 | CCNC | 1.119489673 | 0.067201 |
| 449 | 3199 | NF2 | 1.119765637 | 0.070805 |
| 450 | 3309 | CCND2 | 1.121333431 | 0.146937 |
| 451 | 3143 | NM_017596 | 1.121623368 | 0.07995 |
| 452 | 3208 | ZW10 | 1.121902285 | 0.144279 |
| 453 | 3753 | CDK5 | 1.123427629 | 0.130821 |
| 454 | 3001 | PRKY | 1.125456942 | 0.164937 |
| 455 | 3729 | RYK | 1.125623162 | 0.196578 |
| 456 | 3156 | MSH2 | 1.125991819 | 0.128643 |
| 457 | 3253 | PRKCA | 1.126352597 | 0.097687 |
| 458 | 3607 | TLE1 | 1.126388877 | 0.255505 |
| 459 | 3818 | AI338451 | 1.126447243 | 0.085307 |
| 460 | 3530 | NOTCH1 | 1.127939559 | 0.128155 |
| 461 | 3141 | NM_145754 | 1.129479267 | 0.026346 |
| 462 | 3768 | ARAF1 | 1.129705288 | 0.086972 |
| 463 | 3596 | SHMT1 | 1.129818514 | 0.046177 |
| 464 | 3653 | NPR2 | 1.129853377 | 0.184709 |
| 465 | 3640 | STAT5B | 1.132589635 | 0.299667 |
| 466 | 2924 | STK25 | 1.13270396 | 0.083695 |
| 467 | 3356 | TUBG1 | 1.133623741 | 0.248371 |
| 468 | 3008 | SGK2 | 1.135373086 | 0.09569 |
| 469 | 3499 | GRB2 | 1.135404457 | 0.174583 |
| 470 | 3506 | XM_095827 | 1.135823602 | 0.058483 |
| 471 | 3770 | TGFBR2 | 1.136061775 | 0.283098 |
| 472 | 3441 | PRKCI | 1.137712494 | 0.174946 |
| 473 | 3609 | FZD3 | 1.138082685 | 0.180803 |
| 474 | 3370 | AR | 1.139336644 | 0.114355 |
| 475 | 3126 | KIF3B | 1.139588548 | 0.094914 |
| 476 | 3508 | KIF25 | 1.140573718 | 0.158738 |
| 477 | 3233 | ROCK1 | 1.140584559 | 0.236383 |
| 478 | 2941 | DYRK3 | 1.142052549 | 0.138936 |
| 479 | 3336 | CDC37 | 1.142173919 | 0.132765 |
| 480 | 3741 | RPS6KB2 | 1.142253082 | 0.114889 |
| 481 | 3546 | INPP5D | 1.142646282 | 0.17732 |
| 482 | 3350 | ADA | 1.14270522 | 0.202027 |
| 483 | 3759 | NM_006622 | 1.143528436 | 0.053271 |
| 484 | 3149 | TP53 | 1.144116968 | 0.028664 |
| 485 | 3310 | CDC34 | 1.145001246 | 0.124753 |
| 486 | 3267 | CCNH | 1.145203121 | 0.081778 |
| 487 | 3638 | STAT5A | 1.145284022 | 0.182015 |
| 488 | 3564 | RALBP1 | 1.145302766 | 0.187726 |
| 489 | 3360 | RRM2 | 1.145371751 | 0.106232 |
| 490 | 3662 | LCK | 1.145638747 | 0.091184 |
| 491 | 3223 | NM_016263 | 1.145667614 | 0.160673 |
| 492 | 3408 | PIN1 | 1.146539359 | 0.100954 |
| 493 | 2986 | ACVR2 | 1.146661813 | 0.10512 |
| 494 | 3304 | CCNE2 | 1.146795654 | 0.102769 |
| 495 | 2997 | MST1R | 1.147221866 | 0.283163 |
| 496 | 3194 | RARB | 1.14777913 | 0.330433 |
| 497 | 3669 | NTRK3 | 1.148222927 | 0.037566 |
| 498 | 3616 | FZD1 | 1.148473923 | 0.242876 |
| 499 | 3255 | CDK7 | 1.148553587 | 0.16951 |
| 500 | 3238 | MAP3K3 | 1.1489897 | 0.067123 |
| 501 | 3613 | DVL1 | 1.14901778 | 0.082647 |
| 502 | 3614 | CTNND2 | 1.14937005 | 0.187988 |
| 503 | 3318 | CUL2 | 1.150267783 | 0.078013 |
| 504 | 3644 | EPHB1 | 1.15071896 | 0.123257 |
| 505 | 3567 | SHC1 | 1.151587989 | 0.124227 |
| 506 | 3116 | KIF5A | 1.152039039 | 0.280422 |
| 507 | 3148 | LIG1 | 1.152183128 | 0.190895 |
| 508 | 3765 | CREBBP | 1.154589409 | 0.128712 |
| 509 | 3232 | KDR | 1.156581097 | 0.11153 |
| 510 | 3748 | NM_016507 | 1.157187762 | 0.187551 |
| 511 | 3428 | ECT2 | 1.157383105 | 0.171141 |
| 512 | 3649 | CAMK2B | 1.157415503 | 0.051472 |
| 513 | 3426 | TK1 | 1.158048559 | 0.161458 |
| 514 | 3250 | CHEK2 | 1.158473201 | 0.099737 |
| 515 | 3636 | STAT2 | 1.158495567 | 0.161875 |
| 516 | 3187 | WNT7B | 1.158590559 | 0.024217 |
| 517 | 3505 | STK6 | 1.159146577 | 0.058438 |
| 518 | 3341 | APLP2 | 1.160801239 | 0.196169 |
| 519 | 3606 | CREBBP | 1.161326405 | 0.064695 |
| 520 | 3263 | CDC2 | 1.161570849 | 0.095606 |
| 521 | 2939 | TLK1 | 1.163052719 | 0.110779 |
| 522 | 3123 | AKT3 | 1.163145874 | 0.306815 |
| 523 | 3615 | FZD2 | 1.16322422 | 0.169339 |
| 524 | 3688 | GUCY2D | 1.164321663 | 0.152801 |
| 525 | 3379 | NM_032525 | 1.16446975 | 0.074802 |
| 526 | 3710 | GPRK2L | 1.164900142 | 0.112446 |
| 527 | 3611 | CTNNAL1 | 1.166257931 | 0.037335 |
| 528 | 3521 | MET | 1.168013918 | 0.232923 |
| 529 | 3659 | NM_015978 | 1.169523683 | 0.056392 |
| 530 | 3582 | GRAP2 | 1.170573492 | 0.055118 |
| 531 | 3562 | RASD1 | 1.171229699 | 0.101195 |
| 532 | 3723 | NM_018401 | 1.1718512 | 0.239747 |
| 533 | 3130 | FRAP1 | 1.172072928 | 0.029779 |
| 534 | 3772 | RPS6KB1 | 1.172823934 | 0.066518 |
| 535 | 3333 | CCNT2 | 1.174235642 | 0.214732 |
| 536 | 3501 | RPS6KA2 | 1.175781534 | 0.193038 |
| 537 | 3803 | MPHOSPH1 | 1.176011971 | 0.128864 |
| 538 | 3248 | JAK2 | 1.176020977 | 0.176345 |
| 539 | 3538 | NFKB2 | 1.176129803 | 0.052353 |
| 540 | 3732 | CSNK2A2 | 1.177521611 | 0.231267 |
| 541 | 3730 | TESK1 | 1.177904268 | 0.212526 |
| 542 | 2989 | ACVR1B | 1.178578161 | 0.217492 |
| 543 | 3327 | CDC45L | 1.180204158 | 0.299357 |
| 544 | 3301 | CCNB1 | 1.180864124 | 0.162992 |
| 545 | 3092 | KIF12 | 1.181937605 | 0.088708 |
| 546 | 3239 | CDK6 | 1.182157904 | 0.061044 |
| 547 | 3190 | WNT4 | 1.182697676 | 0.072644 |
| 548 | 3811 | NM_152524 | 1.183191701 | 0.14187 |
| 549 | 2940 | DCAMKL1 | 1.184491938 | 0.124698 |
| 550 | 3761 | WT1 | 1.184547796 | 0.16129 |
| 551 | 3439 | EGR2 | 1.185671136 | 0.105284 |
| 552 | 3295 | CDK2AP1 | 1.187045536 | 0.206856 |
| 553 | 3817 | NM_019013 | 1.187780743 | 0.082116 |
| 554 | 3754 | CDKL2 | 1.188123077 | 0.122877 |
| 555 | 3663 | ALS2CR2 | 1.188246404 | 0.140777 |
| 556 | 3718 | PTK6 | 1.188784586 | 0.067781 |
| 557 | 3236 | PTK2B | 1.189818532 | 0.352399 |
| 558 | 3475 | EPHB4 | 1.189888477 | 0.105406 |
| 559 | 3211 | BUB1B | 1.189896824 | 0.292886 |
| 560 | 3411 | HIF1A | 1.19069666 | 0.245883 |
| 561 | 2927 | MAPK13 | 1.190710916 | 0.129633 |
| 562 | 3264 | CDK3 | 1.191042267 | 0.100335 |
| 563 | 3207 | MAD1L1 | 1.191232546 | 0.092266 |
| 564 | 3372 | TUBA8 | 1.191540593 | 0.058385 |
| 565 | 3349 | IMPDH1 | 1.191967983 | 0.225505 |
| 566 | 3353 | PGR | 1.192360399 | 0.019529 |
| 567 | 3252 | NEK2 | 1.192601635 | 0.282445 |
| 568 | 3515 | PDGFRB | 1.192640873 | 0.057678 |
| 569 | 3216 | CDC20 | 1.193040181 | 0.077143 |
| 570 | 2971 | DAPK2 | 1.19306626 | 0.085366 |
| 571 | 3552 | PDK1 | 1.194218291 | 0.064673 |
| 572 | 3823 | NM_017779 | 1.194861064 | 0.138396 |
| 573 | 3528 | TCF3 | 1.195851197 | 0.12389 |
| 574 | 3201 | RARA | 1.19739521 | 0.087982 |
| 575 | 2945 | CDC42BPB | 1.19753147 | 0.087566 |
| 576 | 3634 | BTRC | 1.201076339 | 0.175356 |
| 577 | 3377 | NM_006088 | 1.202720091 | 0.096411 |
| 578 | 3781 | SRC | 1.202931814 | 0.331139 |
| 579 | 3516 | ARHA | 1.203109256 | 0.159342 |
| 580 | 3700 | AB037782 | 1.204329771 | 0.402706 |
| 581 | 3699 | NM_032844 | 1.207623266 | 0.248703 |
| 582 | 2931 | MAP4K3 | 1.211854633 | 0.149673 |
| 583 | 3189 | MYB | 1.212003569 | 0.117606 |
| 584 | 3586 | RASA2 | 1.212166142 | 0.210711 |
| 585 | 3836 | TP53 | 1.212183899 | 0.169152 |
| 586 | 3206 | ANAPC5 | 1.213545746 | 0.079256 |
| 587 | 3701 | STK10 | 1.214412753 | 0.23119 |
| 588 | 3210 | NM_013366 | 1.21450599 | 0.307913 |
| 589 | 3472 | MAP3K5 | 1.215042561 | 0.128134 |
| 590 | 3371 | NM_006087 | 1.216059457 | 0.10804 |
| 591 | 3825 | NM_152562 | 1.216108937 | 0.153345 |
| 592 | 3106 | KIF9 | 1.217161737 | 0.277445 |
| 593 | 3249 | MAP2K6 | 1.217382649 | 0.23408 |
| 594 | 3186 | ETS1 | 1.218428895 | 0.125809 |
| 595 | 3541 | PKD2 | 1.220374384 | 0.252301 |
| 596 | 3654 | VRK2 | 1.221266095 | 0.180133 |
| 597 | 3151 | MLH1 | 1.221977195 | 0.100529 |
| 598 | 3325 | CDKN1C | 1.222573895 | 0.183555 |
| 599 | 3774 | CDC45L | 1.223373496 | 0.170335 |
| 600 | 3354 | RRM1 | 1.224155502 | 0.163218 |
| 601 | 3225 | NM_013367 | 1.226360075 | 0.377268 |
| 602 | 3837 | PRKAA1 | 1.226867311 | 0.260099 |
| 603 | 2930 | ITK | 1.227438086 | 0.134102 |
| 604 | 3118 | NM_032559 | 1.22825648 | 0.037564 |
| 605 | 3316 | CCNA1 | 1.228667093 | 0.203935 |
| 606 | 3651 | VRK1 | 1.229029159 | 0.155208 |
| 607 | 3368 | TOP3A | 1.229032423 | 0.14134 |
| 608 | 3376 | AGA | 1.231363135 | 0.128058 |
| 609 | 3735 | PRKACB | 1.231534849 | 0.092436 |
| 610 | 3007 | MAP3K14 | 1.234231675 | 0.17551 |
| 611 | 3420 | NM_014109 | 1.234890989 | 0.370528 |
| 612 | 3131 | KIF1B | 1.235185989 | 0.242087 |
| 613 | 3444 | NEK3 | 1.23520517 | 0.358385 |
| 614 | 2919 | OSR1 | 1.237086236 | 0.106562 |
| 615 | 3128 | AKT2 | 1.242407611 | 0.124394 |
| 616 | 3810 | AI278633 | 1.243126735 | 0.165137 |
| 617 | 3337 | CDKN1A | 1.244628023 | 0.018797 |
| 618 | 3091 | KIFC3 | 1.244757733 | 0.153624 |
| 619 | 3191 | WNT2 | 1.244817311 | 0.187282 |
| 620 | 3146 | KIF21A | 1.24572579 | 0.041267 |
| 621 | 3220 | ANAPC11 | 1.246197987 | 0.17988 |
| 622 | 3785 | GRB2 | 1.248971557 | 0.108491 |
| 623 | 3195 | CDH1 | 1.250259859 | 0.186152 |
| 624 | 3500 | SGK | 1.251914788 | 0.059299 |
| 625 | 3103 | PIK3CA | 1.252248606 | 0.068043 |
| 626 | 3507 | NM_145754 | 1.252689721 | 0.222951 |
| 627 | 3565 | RAB2 | 1.253586902 | 0.186552 |
| 628 | 3462 | TGFBR2 | 1.256459996 | 0.136016 |
| 629 | 3229 | PRKCL1 | 1.256649876 | 0.153419 |
| 630 | 3790 | ERBB3 | 1.260353633 | 0.104586 |
| 631 | 3704 | ACVR2B | 1.261857433 | 0.075994 |
| 632 | 3340 | CENPH | 1.262786326 | 0.131215 |
| 633 | 3598 | PCNA | 1.265667779 | 0.116032 |
| 634 | 2967 | NM_016653 | 1.266923195 | 0.232771 |
| 635 | 3725 | EPHA4 | 1.267438993 | 0.19056 |
| 636 | 2932 | MAPKAPK3 | 1.268643945 | 0.025332 |
| 637 | 3167 | S100A2 | 1.270859999 | 0.069296 |
| 638 | 2994 | MATK | 1.271154735 | 0.128018 |
| 639 | 3315 | CCT4 | 1.272355192 | 0.309065 |
| 640 | 3344 | CDKL1 | 1.272536383 | 0.273155 |
| 641 | 3689 | BLK | 1.27387895 | 0.218306 |
| 642 | 3104 | CDK4 | 1.276578446 | 0.161716 |
| 643 | 3604 | TK2 | 1.277912947 | 0.101801 |
| 644 | 3209 | MAD2L2 | 1.278038114 | 0.253976 |
| 645 | 3554 | PIK3R3 | 1.280284314 | 0.228353 |
| 646 | 3218 | CDC23 | 1.280483947 | 0.334381 |
| 647 | 3670 | MAP3K10 | 1.280649754 | 0.129166 |
| 648 | 3532 | NM_019089 | 1.280872331 | 0.138131 |
| 649 | 3558 | RALA | 1.282343213 | 0.193164 |
| 650 | 3440 | FGFR3 | 1.283277949 | 0.278946 |
| 651 | 3779 | CTNNA1 | 1.285066069 | 0.05853 |
| 652 | 3312 | CUL3 | 1.286086663 | 0.095171 |
| 653 | 3111 | KIF5B | 1.286155963 | 0.045454 |
| 654 | 3320 | CCND3 | 1.286427699 | 0.049781 |
| 655 | 3493 | MAPK9 | 1.286708555 | 0.204254 |
| 656 | 3463 | TEC | 1.286731353 | 0.116346 |
| 657 | 3198 | ICAM1 | 1.287087211 | 0.105164 |
| 658 | 2933 | MAP4K5 | 1.288848714 | 0.19588 |
| 659 | 2995 | PTK2 | 1.289781227 | 0.122006 |
| 660 | 3637 | STAT4 | 1.291478071 | 0.126765 |
| 661 | 3089 | KIFC1 | 1.292296631 | 0.104185 |
| 662 | 3330 | CDK9 | 1.293189989 | 0.200332 |
| 663 | 3588 | RHEB | 1.295786915 | 0.113922 |
| 664 | 3589 | SOS1 | 1.300834959 | 0.028846 |
| 665 | 3418 | CENPA | 1.300851356 | 0.224648 |
| 666 | 3314 | CCNG1 | 1.302132075 | 0.167018 |
| 667 | 3697 | CAMK2G | 1.305521288 | 0.141582 |
| 668 | 3620 | AXIN2 | 1.306881235 | 0.175725 |
| 669 | 2921 | RPS6KA5 | 1.307895788 | 0.116976 |
| 670 | 3157 | NF1 | 1.312364005 | 0.21979 |
| 671 | 3172 | PLAU | 1.314219765 | 0.221395 |
| 672 | 3221 | TOP3B | 1.316767724 | 0.153732 |
| 673 | 3529 | DTX1 | 1.317104131 | 0.100042 |
| 674 | 3520 | NRAS | 1.318162379 | 0.337798 |
| 675 | 3138 | KIF17 | 1.319021332 | 0.047239 |
| 676 | 3466 | JAK3 | 1.324590857 | 0.341923 |
| 677 | 3447 | PRKCM | 1.325852782 | 0.090164 |
| 678 | 3396 | HDAC10 | 1.327036067 | 0.095421 |
| 679 | 3405 | HDAC8 | 1.328986383 | 0.137456 |
| 680 | 2956 | PRKCL2 | 1.329759987 | 0.277544 |
| 681 | 3771 | PIK3CA | 1.330927854 | 0.318605 |
| 682 | 3100 | GSK3B | 1.332661843 | 0.172085 |
| 683 | 3140 | XM_089006 | 1.334634688 | 0.210199 |
| 684 | 3417 | HDAC3 | 1.334739136 | 0.213735 |
| 685 | 2912 | MPHOSPH1 | 1.335606817 | 0.192246 |
| 686 | 3453 | MAP2K2 | 1.337178184 | 0.231133 |
| 687 | 3777 | ABL1 | 1.337946355 | 0.13381 |
| 688 | 2991 | NPR1 | 1.339668528 | 0.213719 |
| 689 | 3234 | CDK2 | 1.341378632 | 0.327603 |
| 690 | 3617 | CTNNBIP1 | 1.344129969 | 0.172119 |
| 691 | 3217 | NM_014885 | 1.346642104 | 0.37578 |
| 692 | 3632 | WISP1 | 1.34798621 | 0.267584 |
| 693 | 3404 | PPARG | 1.350608226 | 0.328799 |
| 694 | 3834 | CHEK1 | 1.353682807 | 0.177332 |
| 695 | 3244 | PRKCZ | 1.354506447 | 0.423569 |
| 696 | 3242 | PRKCB1 | 1.356110177 | 0.177856 |
| 697 | 2998 | MAPK7 | 1.357915027 | 0.320918 |
| 698 | 3227 | NUMA1 | 1.358033567 | 0.336206 |
| 699 | 3676 | MAP4K1 | 1.360624202 | 0.35665 |
| 700 | 3087 | PTEN | 1.361043077 | 0.221803 |
| 701 | 3734 | BMPR1B | 1.362751745 | 0.19663 |
| 702 | 3569 | RASGRP2 | 1.362852713 | 0.083746 |
| 703 | 2953 | MAPK11 | 1.367417583 | 0.335411 |
| 704 | 3355 | GUK1 | 1.368854888 | 0.121328 |
| 705 | 3713 | PRKG2 | 1.370762753 | 0.096281 |
| 706 | 3415 | HSPCA | 1.373102391 | 0.311637 |
| 707 | 3212 | NM_022662 | 1.373845206 | 0.3643 |
| 708 | 3789 | ELK1 | 1.378293297 | 0.119276 |
| 709 | 3395 | HDAC5 | 1.380918509 | 0.316118 |
| 710 | 3448 | NM_016231 | 1.382981639 | 0.250346 |
| 711 | 3737 | NM_016457 | 1.384393078 | 0.35016 |
| 712 | 3456 | FLT1 | 1.387001071 | 0.117573 |
| 713 | 3696 | NM_016281 | 1.392513247 | 0.179922 |
| 714 | 3124 | KIF4A | 1.392774473 | 0.395931 |
| 715 | 3451 | MAP3K4 | 1.39359615 | 0.215328 |
| 716 | 3738 | PRKWNK3 | 1.395197042 | 0.116947 |
| 717 | 3719 | BMPR2 | 1.395676978 | 0.083941 |
| 718 | 3429 | MCM6 | 1.399624047 | 0.422475 |
| 719 | 3243 | NM_004203 | 1.401278663 | 0.303675 |
| 720 | 3660 | DMPK | 1.402604745 | 0.203011 |
| 721 | 3084 | KIF14 | 1.405667444 | 0.022909 |
| 722 | 3574 | SH3KBP1 | 1.408096057 | 0.080055 |
| 723 | 3137 | KIF26A | 1.410397298 | 0.209271 |
| 724 | 3671 | STK4 | 1.410482157 | 0.306699 |
| 725 | 3202 | MCC | 1.410775773 | 0.285878 |
| 726 | 3134 | XM_170783 | 1.415482722 | 0.226917 |
| 727 | 3204 | CDC16 | 1.415780291 | 0.221962 |
| 728 | 3121 | PIK3C2A | 1.415950012 | 0.152356 |
| 729 | 3321 | CDKN3 | 1.416176725 | 0.03826 |
| 730 | 2951 | AJ311798 | 1.416769938 | 0.177239 |
| 731 | 3504 | PIK3CB | 1.417592955 | 0.174632 |
| 732 | 2955 | PAK1 | 1.418341722 | 0.07362 |
| 733 | 3612 | TCF1 | 1.421215206 | 0.099637 |
| 734 | 3655 | CAMK2D | 1.422993988 | 0.227042 |
| 735 | 3135 | XM_064050 | 1.42777471 | 0.205042 |
| 736 | 3400 | BCL2 | 1.432342001 | 0.20388 |
| 737 | 3794 | WASL | 1.432665996 | 0.093756 |
| 738 | 3667 | NM_016542 | 1.434249905 | 0.174478 |
| 739 | 3407 | HDAC9 | 1.437540011 | 0.194891 |
| 740 | 3430 | STMN1 | 1.437943227 | 0.313077 |
| 741 | 3698 | ADRBK2 | 1.440932223 | 0.248217 |
| 742 | 3547 | FOXO1A | 1.461102151 | 0.113568 |
| 743 | 3265 | RAF1 | 1.463315846 | 0.191102 |
| 744 | 3690 | PRKAA2 | 1.470696795 | 0.233827 |
| 745 | 3510 | CDK4 | 1.487289818 | 0.089318 |
| 746 | 3254 | CSF1R | 1.487946096 | 0.480379 |
| 747 | 3622 | FZD9 | 1.49526423 | 0.086599 |
| 748 | 3544 | IRS1 | 1.495662211 | 0.040512 |
| 749 | 2948 | MYO3A | 1.4970851 | 0.081259 |
| 750 | 3467 | MAP2K7 | 1.497928287 | 0.281253 |
| 751 | 3096 | AKT1 | 1.498269053 | 0.114084 |
| 752 | 2968 | STK17B | 1.504346366 | 0.413498 |
| 753 | 3402 | HDAC4 | 1.508119762 | 0.213089 |
| 754 | 3764 | NOTCH4 | 1.510551197 | 0.081586 |
| 755 | 3621 | CTNNA2 | 1.511056295 | 0.111649 |
| 756 | 3168 | DCC | 1.513409582 | 0.192599 |
| 757 | 2701 | 2701 | 1.51348211 | 0.083391 |
| 758 | 3629 | AXIN1 | 1.520734118 | 0.174178 |
| 759 | 3361 | IMPDH2 | 1.523631011 | 0.067873 |
| 760 | 3129 | STK6 | 1.527103437 | 0.134504 |
| 761 | 3679 | CLK2 | 1.53626119 | 0.44738 |
| 762 | 3709 | X95425 | 1.537827387 | 0.437676 |
| 763 | 2962 | MAP4K2 | 1.547893113 | 0.329021 |
| 764 | 3442 | ERBB4 | 1.551008953 | 0.146803 |
| 765 | 3247 | NM_018492 | 1.552802846 | 0.137033 |
| 766 | 3720 | AB002301 | 1.553258633 | 0.313891 |
| 767 | 3584 | RASAL2 | 1.563405608 | 0.137336 |
| 768 | 3299 | CUL1 | 1.589913659 | 0.169909 |
| 769 | 3522 | KRAS2 | 1.590661017 | 0.06841 |
| 770 | 3590 | ARHGEF2 | 1.597950927 | 0.252526 |
| 771 | 3406 | TERT | 1.600169172 | 0.094096 |
| 772 | 3259 | MAPK8 | 1.601990057 | 0.333883 |
| 773 | 3369 | NM_007027 | 1.605090071 | 0.162172 |
| 774 | 3787 | FZD4 | 1.621497881 | 0.053639 |
| 775 | 2929 | CHUK | 1.646057679 | 0.111716 |
| 776 | 3468 | ABL2 | 1.652007602 | 0.180802 |
| 777 | 2988 | FRK | 1.653152871 | 0.298882 |
| 778 | 3758 | RAD51L1 | 1.662423293 | 0.135675 |
| 779 | 3531 | NM_021170 | 1.666989154 | 0.094206 |
| 780 | 3155 | ATR | 1.680571715 | 0.388687 |
| 781 | 3747 | GSK3A | 1.688637713 | 0.38953 |
| 782 | 3144 | KIF4B | 1.695873891 | 0.467258 |
| 783 | 3235 | CHEK1 | 1.697910825 | 0.356224 |
| 784 | 3313 | CCNG2 | 1.703651114 | 0.216266 |
| 785 | 3004 | MAP3K1 | 1.721492222 | 0.376438 |
| 786 | 3619 | FRAT1 | 1.761446915 | 0.292031 |
| 787 | 3192 | WNT1 | 1.765037748 | 0.394063 |
| 788 | 3673 | DDR1 | 1.770053978 | 0.2338 |
| 789 | 3358 | TOP2B | 1.800293702 | 0.195754 |
| 790 | 2981 | ALK | 1.84348901 | 0.208338 |
| 791 | 2958 | PRKACA | 1.889142934 | 0.494773 |
| 792 | 3152 | APC | 1.894694006 | 0.191358 |
| 793 | 3712 | RPS6KA6 | 1.957145081 | 0.421292 |
| 794 | 3436 | BRAF | 1.999825737 | 1.173208 |
| 795 | 3727 | GPRK6 | 2.044605743 | 6.256806 |
| 796 | 3780 | MCM3 | 2.062893191 | 0.187038 |
| 797 | 3329 | CDC42 | 2.131693629 | 0.483392 |
| 798 | 3095 | KIF2C | 2.163834467 | 0.289685 |
| 799 | 3098 | CENPE | 2.170456559 | 0.120025 |
| 800 | 3331 | CDC25B | 2.199340751 | 0.484716 |
| 801 | 3706 | C20orf97 | 2.377822809 | 0.678329 |
| 802 | 3580 | ELK1 | 2.456195789 | 0.434043 |
| 803 | 3241 | WEE1 | 2.66755235 | 0.625231 |
| 804 | 3642 | EPHB3 | 2.758093154 | 0.565256 |
| 805 | 3158 | BRCA1 | 2.878071685 | 0.418358 |
| 806 | 3150 | BRCA2 | 11.61633698 | 1.101248 |

TABLE IIB
Average fold sensitization by camptothecin

| | | | fold |
|---|---|---|---|
| Gene ID | BIOID | GENE | sensitization |
| 1 | 63 | M15077 | 1 |
| 2 | 2514 | PLK | 0.029197 |
| 3 | 2540 | 2540 | #DIV/0! |
| 4 | 2541 | 2541 | 0.860453 |
| 5 | 2701 | 2701 | 0.034091 |
| 6 | 2702 | 2702 | 0.432441 |
| 7 | 3391 | PLK | 0.052632 |
| 8 | 3534 | PLK | 0.083815 |
| 9 | 3099 | PLK | 0.090142 |
| 10 | 3006 | PLK | 0.09146 |
| 11 | 3266 | PLK | 0.096774 |
| 12 | 3433 | PLK | 0.13 |
| 13 | 3322 | CCNA2 | 0.264029 |
| 14 | 3154 | MADH4 | 0.361653 |
| 15 | 3518 | NFKB1 | 0.372726 |
| 16 | 3600 | RRM2B | 0.381056 |
| 17 | 3184 | TSG101 | 0.432287 |
| 18 | 3348 | DCK | 0.446467 |
| 19 | 3332 | CDC5L | 0.451264 |
| 20 | 3812 | CDCA8 | 0.453177 |
| 21 | 3423 | NM_006101 | 0.478261 |
| 22 | 3464 | INSR | 0.480578 |
| 23 | 2961 | PIM1 | 0.51581 |
| 24 | 3661 | FGR | 0.517647 |
| 25 | 3171 | VHL | 0.524194 |
| 26 | 3809 | CDCA3 | 0.529046 |
| 27 | 3525 | NOTCH4 | 0.534058 |
| 28 | 3093 | PIK3CG | 0.557692 |
| 29 | 3740 | STK35 | 0.55782 |
| 30 | 3435 | FLT3 | 0.56 |
| 31 | 3805 | C20orf1 | 0.564035 |
| 32 | 3219 | CENPC1 | 0.575465 |
| 33 | 3003 | FER | 0.579832 |
| 34 | 3183 | NM_005200 | 0.580153 |
| 35 | 3374 | POLR2A | 0.583796 |
| 36 | 3601 | POLE | 0.588331 |
| 37 | 3112 | KNSL7 | 0.597685 |
| 38 | 3489 | SRC | 0.606833 |
| 39 | 3478 | EPHA2 | 0.608258 |
| 40 | 3422 | SMC4L1 | 0.608696 |
| 41 | 3357 | PRIM2A | 0.611218 |
| 42 | 3262 | LATS1 | 0.613321 |
| 43 | 2987 | RNASEL | 0.617089 |
| 44 | 3123 | AKT3 | 0.618574 |
| 45 | 3687 | CAMK4 | 0.61913 |
| 46 | 3303 | CDKN2D | 0.625741 |
| 47 | 2966 | NM_033266 | 0.627321 |
| 48 | 3226 | RBX1 | 0.632166 |
| 49 | 3509 | KIF21A | 0.634426 |
| 50 | 2999 | FES | 0.634596 |
| 51 | 3517 | MYC | 0.637624 |
| 52 | 3592 | SOS2 | 0.640343 |
| 53 | 3139 | XM_095827 | 0.642535 |
| 54 | 3105 | BUB1 | 0.643861 |
| 55 | 3397 | BUB3 | 0.644077 |
| 56 | 3267 | CCNH | 0.64503 |
| 57 | 2975 | NEK4 | 0.645485 |
| 58 | 3766 | S100A2 | 0.646739 |
| 59 | 2936 | SGKL | 0.64695 |
| 60 | 3524 | NOTCH3 | 0.647109 |
| 61 | 3806 | C10orf3 | 0.64878 |
| 62 | 3448 | NM_016231 | 0.654135 |
| 63 | 3461 | KIT | 0.662033 |
| 64 | 3501 | RPS6KA2 | 0.667039 |
| 65 | 3494 | MAPK4 | 0.668898 |
| 66 | 3251 | ABL1 | 0.675159 |
| 67 | 3103 | PIK3CA | 0.679543 |
| 68 | 3572 | RASA3 | 0.681105 |
| 69 | 3246 | RPS6KB1 | 0.681548 |
| 70 | 3230 | MAP2K1 | 0.683985 |
| 71 | 3733 | MYLK2 | 0.684534 |
| 72 | 3491 | PRKCE | 0.685882 |
| 73 | 2982 | CDC2L2 | 0.687831 |
| 74 | 3542 | PPP2CA | 0.690237 |
| 75 | 3350 | ADA | 0.692046 |
| 76 | 3651 | VRK1 | 0.692308 |
| 77 | 2937 | NM_025052 | 0.693089 |
| 78 | 3007 | MAP3K14 | 0.694169 |
| 79 | 3751 | BCR | 0.694278 |
| 80 | 3410 | RPS27 | 0.695238 |
| 81 | 3240 | MAPK12 | 0.696498 |
| 82 | 2949 | MYO3B | 0.698039 |
| 83 | 3413 | KIF23 | 0.701413 |
| 84 | 3773 | WNT2 | 0.702997 |
| 85 | 3762 | MCC | 0.706533 |
| 86 | 3731 | CSNK1E | 0.707275 |
| 87 | 3778 | VHL | 0.707386 |
| 88 | 3476 | PRKCD | 0.708251 |
| 89 | 3754 | CDKL2 | 0.712665 |
| 90 | 3741 | RPS6KB2 | 0.715261 |
| 91 | 3744 | PRKWNK4 | 0.716089 |
| 92 | 3148 | LIG1 | 0.71816 |
| 93 | 2964 | RIPK2 | 0.71873 |
| 94 | 3486 | RPS6KA3 | 0.71875 |
| 95 | 3772 | RPS6KB1 | 0.722315 |
| 96 | 3193 | FGF3 | 0.723179 |
| 97 | 3363 | GART | 0.723732 |
| 98 | 3438 | DTR | 0.725061 |
| 99 | 3351 | ESR1 | 0.725395 |
| 100 | 3416 | IKBKE | 0.726044 |
| 101 | 2972 | KSR | 0.727171 |
| 102 | 3326 | CCT7 | 0.727769 |
| 103 | 3648 | CLK1 | 0.728232 |
| 104 | 3401 | HDAC1 | 0.728268 |
| 105 | 3498 | CSNK1A1 | 0.728714 |
| 106 | 2976 | NEK7 | 0.729805 |
| 107 | 3347 | TOP1 | 0.731826 |
| 108 | 3236 | PTK2B | 0.736339 |
| 109 | 3256 | CDC2L1 | 0.738272 |
| 110 | 3606 | CREBBP | 0.738487 |
| 111 | 3657 | PCTK1 | 0.739866 |
| 112 | 3452 | JAK1 | 0.745847 |
| 113 | 3250 | CHEK2 | 0.745919 |
| 114 | 3200 | REL | 0.746919 |
| 115 | 3403 | AURKC | 0.747841 |
| 116 | 3663 | ALS2CR2 | 0.749671 |
| 117 | 3208 | ZW10 | 0.75 |
| 118 | 3647 | YES1 | 0.750637 |
| 119 | 3466 | JAK3 | 0.750708 |
| 120 | 3196 | ARHI | 0.75402 |
| 121 | 3757 | CLK4 | 0.757793 |
| 122 | 3434 | DDX6 | 0.758671 |
| 123 | 3460 | CSK | 0.759454 |
| 124 | 3722 | TLK2 | 0.761568 |
| 125 | 3306 | CDC14A | 0.761859 |
| 126 | 3412 | KIF25 | 0.761959 |
| 127 | 2926 | AF172264 | 0.762856 |
| 128 | 3382 | TUBB | 0.763294 |
| 129 | 2965 | NM_014720 | 0.763727 |
| 130 | 3625 | CTBP2 | 0.763827 |
| 131 | 3702 | MAP3K13 | 0.764125 |
| 132 | 3650 | NM_025195 | 0.764957 |
| 133 | 3323 | CDK5R1 | 0.765293 |
| 134 | 3653 | NPR2 | 0.765609 |
| 135 | 2997 | MST1R | 0.767068 |
| 136 | 3658 | STK18 | 0.768411 |
| 137 | 3739 | NM_017886 | 0.768662 |
| 138 | 2993 | SRMS | 0.768678 |
| 139 | 3166 | PMS1 | 0.769717 |
| 140 | 3775 | NOTCH1 | 0.770983 |
| 141 | 3469 | AAK1 | 0.772082 |
| 142 | 3833 | ATR | 0.772423 |
| 143 | 3211 | BUB1B | 0.773389 |
| 144 | 3557 | JUND | 0.773496 |
| 145 | 3179 | PDGFB | 0.777522 |
| 146 | 3674 | CSNK1D | 0.779923 |
| 147 | 3566 | JUN | 0.780371 |
| 148 | 3341 | APLP2 | 0.781888 |
| 149 | 3188 | PMS2 | 0.785359 |
| 150 | 3633 | CTBP1 | 0.786631 |
| 151 | 2923 | ERN1 | 0.787194 |
| 152 | 3086 | KIF11 | 0.787201 |
| 153 | 3688 | GUCY2D | 0.787284 |
| 154 | 3605 | FZD4 | 0.787879 |
| 155 | 3640 | STAT5B | 0.789018 |
| 156 | 2974 | NEK11 | 0.791024 |
| 157 | 3473 | DAPK1 | 0.791285 |
| 158 | 3376 | AGA | 0.791586 |
| 159 | 3263 | CDC2 | 0.792593 |
| 160 | 3475 | EPHB4 | 0.797463 |
| 161 | 3346 | CCNK | 0.79871 |
| 162 | 3298 | CCNE1 | 0.800418 |
| 163 | 3359 | TUBA1 | 0.801205 |
| 164 | 3609 | FZD3 | 0.806613 |
| 165 | 3201 | RARA | 0.808157 |
| 166 | 3394 | HDAC6 | 0.810106 |
| 167 | 3770 | TGFBR2 | 0.810897 |
| 168 | 3258 | MOS | 0.811566 |
| 169 | 3541 | PKD2 | 0.811594 |
| 170 | 3822 | GTSE1 | 0.814495 |
| 171 | 3450 | MAP3K2 | 0.81592 |
| 172 | 3577 | RAB2L | 0.816 |
| 173 | 3203 | ITGA5 | 0.817391 |
| 174 | 3838 | PRKAA2 | 0.821543 |
| 175 | 3085 | KIF5C | 0.82316 |
| 176 | 3477 | FGFR4 | 0.824427 |
| 177 | 3573 | NM_016848 | 0.824468 |
| 178 | 3836 | TP53 | 0.825022 |
| 179 | 3782 | SOS1 | 0.825161 |
| 180 | 3366 | TYMS | 0.828914 |
| 181 | 3381 | POLR2B | 0.828921 |
| 182 | 3710 | GPRK2L | 0.830756 |
| 183 | 2934 | IRAK2 | 0.830809 |
| 184 | 3364 | HPRT1 | 0.831103 |
| 185 | 3182 | MYCN | 0.831349 |
| 186 | 3783 | KRAS2 | 0.831863 |
| 187 | 3113 | CNK | 0.834672 |
| 188 | 3835 | CHEK2 | 0.836402 |
| 189 | 3680 | CLK3 | 0.836728 |
| 190 | 3131 | KIF1B | 0.83697 |
| 191 | 3088 | KIF13B | 0.838299 |
| 192 | 3581 | RASGRP1 | 0.839735 |
| 193 | 3829 | KIF2C | 0.840215 |
| 194 | 3380 | TUBG2 | 0.840866 |
| 195 | 3334 | CUL4B | 0.842773 |
| 196 | 3746 | HUNK | 0.84279 |
| 197 | 2921 | RPS6KA5 | 0.845122 |
| 198 | 3769 | PLAU | 0.845466 |
| 199 | 2984 | EPHB6 | 0.847067 |
| 200 | 3814 | HMMR | 0.850166 |
| 201 | 3623 | CTNND1 | 0.850309 |
| 202 | 3444 | NEK3 | 0.851852 |
| 203 | 2935 | MAPK6 | 0.852713 |
| 204 | 2996 | MAPK3 | 0.853188 |
| 205 | 2969 | NM_014916 | 0.856081 |
| 206 | 3120 | PIK3CB | 0.856195 |
| 207 | 3107 | KIF2 | 0.856252 |
| 208 | 3502 | PRKCH | 0.856893 |
| 209 | 3763 | NM_016231 | 0.858333 |
| 210 | 3419 | RFC4 | 0.858657 |
| 211 | 3639 | STAT6 | 0.858685 |
| 212 | 2930 | ITK | 0.860156 |
| 213 | 3124 | KIF4A | 0.860439 |
| 214 | 3209 | MAD2L2 | 0.860811 |
| 215 | 3832 | ATM | 0.861555 |
| 216 | 3774 | CDC45L | 0.862319 |
| 217 | 3342 | CCNT1 | 0.86272 |
| 218 | 3430 | STMN1 | 0.864508 |
| 219 | 3802 | NOTCH3 | 0.865116 |
| 220 | 3309 | CCND2 | 0.865741 |
| 221 | 3411 | HIF1A | 0.867769 |
| 222 | 3717 | NTRK2 | 0.867864 |
| 223 | 3465 | EPHA1 | 0.867876 |
| 224 | 3795 | NR4A2 | 0.867991 |
| 225 | 3659 | NM_015978 | 0.868205 |
| 226 | 3643 | DDR2 | 0.868618 |
| 227 | 3392 | BIRC5 | 0.869293 |
| 228 | 3786 | FRAP1 | 0.870607 |
| 229 | 3297 | CCT2 | 0.872024 |
| 230 | 2991 | NPR1 | 0.872727 |
| 231 | 3318 | CUL2 | 0.87438 |
| 232 | 3293 | CDC25A | 0.875 |
| 233 | 3421 | ORC6L | 0.875341 |
| 234 | 3454 | FLT4 | 0.875663 |
| 235 | 2950 | NEK6 | 0.876961 |
| 236 | 3815 | MAPRE2 | 0.877732 |
| 237 | 3831 | CLSPN | 0.878064 |
| 238 | 3232 | KDR | 0.878378 |
| 239 | 3709 | X95425 | 0.879358 |
| 240 | 2929 | CHUK | 0.881491 |
| 241 | 3378 | NM_006009 | 0.882129 |
| 242 | 2952 | PRKG1 | 0.883408 |
| 243 | 3776 | NOTCH2 | 0.88366 |
| 244 | 3356 | TUBG1 | 0.884709 |
| 245 | 3308 | CDKN2B | 0.885077 |
| 246 | 2967 | NM_016653 | 0.886094 |
| 247 | 3591 | RALB | 0.889362 |
| 248 | 3635 | STAT1 | 0.889881 |
| 249 | 3530 | NOTCH1 | 0.890111 |
| 250 | 3750 | CAMK2A | 0.891525 |
| 251 | 3523 | NOTCH2 | 0.893064 |
| 252 | 2980 | RIPK1 | 0.894417 |
| 253 | 3249 | MAP2K6 | 0.895216 |
| 254 | 3589 | SOS1 | 0.895558 |
| 255 | 3587 | VAV3 | 0.896552 |
| 256 | 2968 | STK17B | 0.899438 |
| 257 | 3505 | STK6 | 0.899549 |
| 258 | 3526 | HES6 | 0.899892 |
| 259 | 3261 | ERBB2 | 0.904662 |
| 260 | 3252 | NEK2 | 0.904873 |
| 261 | 3426 | TK1 | 0.906569 |
| 262 | 3328 | CCNC | 0.909091 |
| 263 | 3470 | RPS6KA1 | 0.909627 |
| 264 | 3798 | ACTR2 | 0.910468 |
| 265 | 3595 | FEN1 | 0.910569 |
| 266 | 3597 | SHMT2 | 0.911368 |
| 267 | 3362 | NR3C1 | 0.911404 |
| 268 | 3257 | IGF1R | 0.911442 |
| 269 | 3665 | PAK4 | 0.913313 |
| 270 | 3678 | PFTK1 | 0.913673 |
| 271 | 3344 | CDKL1 | 0.913907 |
| 272 | 3302 | CDK8 | 0.913988 |
| 273 | 3536 | PIK3C3 | 0.916089 |
| 274 | 3685 | LTK | 0.917246 |
| 275 | 3749 | NM_139021 | 0.917681 |
| 276 | 3268 | CDC25C | 0.919654 |
| 277 | 3743 | RAGE | 0.922602 |
| 278 | 3414 | HDAC7A | 0.925495 |
| 279 | 3162 | MADH2 | 0.927277 |
| 280 | 3429 | MCM6 | 0.928214 |
| 281 | 3682 | DYRK1A | 0.928261 |
| 282 | 3585 | GAB1 | 0.928513 |
| 283 | 3549 | PIK3R2 | 0.930054 |
| 284 | 3233 | ROCK1 | 0.930818 |
| 285 | 3315 | CCT4 | 0.931751 |
| 286 | 2990 | NM_015112 | 0.933149 |
| 287 | 3409 | TTK | 0.934641 |
| 288 | 3237 | CDC7 | 0.938429 |
| 289 | 2960 | LYN | 0.938849 |
| 290 | 3664 | STK17A | 0.93923 |
| 291 | 2931 | MAP4K3 | 0.939649 |
| 292 | 3693 | NEK9 | 0.939894 |
| 293 | 3694 | STK38 | 0.941537 |
| 294 | 3000 | BMX | 0.94164 |
| 295 | 3445 | BMPR1A | 0.944154 |
| 296 | 3207 | MAD1L1 | 0.945191 |
| 297 | 3714 | NM_013355 | 0.947122 |
| 298 | 3652 | PDGFRA | 0.947533 |
| 299 | 3631 | WISP2 | 0.948783 |
| 300 | 3799 | ACTR3 | 0.949315 |
| 301 | 2912 | MPHOSPH1 | 0.95053 |
| 302 | 3142 | KIF25 | 0.950655 |
| 303 | 3755 | CDKL3 | 0.950839 |
| 304 | 3231 | ILK | 0.951 |
| 305 | 3155 | ATR | 0.951613 |
| 306 | 3646 | NM_005781 | 0.952566 |
| 307 | 2979 | PAK2 | 0.953323 |
| 308 | 3296 | CDKN2C | 0.954784 |
| 309 | 2983 | GUCY2C | 0.956701 |
| 310 | 3497 | EPHA8 | 0.958146 |
| 311 | 3163 | THRA | 0.959354 |
| 312 | 3471 | MAPK10 | 0.960665 |
| 313 | 2940 | DCAMKL1 | 0.963487 |
| 314 | 3593 | VAV2 | 0.963865 |
| 315 | 3398 | HDAC11 | 0.964784 |
| 316 | 3752 | CCNB3 | 0.964824 |
| 317 | 3641 | TYRO3 | 0.965291 |
| 318 | 3195 | CDH1 | 0.96542 |
| 319 | 3552 | PDK1 | 0.96695 |
| 320 | 3132 | KIF23 | 0.970496 |
| 321 | 2951 | AJ311798 | 0.971591 |
| 322 | 3214 | CENPF | 0.974453 |
| 323 | 3436 | BRAF | 0.97619 |
| 324 | 3104 | CDK4 | 0.976337 |
| 325 | 2959 | PIM2 | 0.977075 |
| 326 | 3228 | CDC27 | 0.978723 |
| 327 | 3570 | RALGDS | 0.979927 |
| 328 | 3826 | NM_015694 | 0.981405 |
| 329 | 3788 | PRKCE | 0.983254 |
| 330 | 2954 | ROCK2 | 0.983541 |
| 331 | 3539 | PLCG2 | 0.985447 |
| 332 | 3732 | CSNK2A2 | 0.985667 |
| 333 | 3759 | NM_006622 | 0.98881 |
| 334 | 2998 | MAPK7 | 0.993015 |
| 335 | 3352 | NR3C2 | 0.996058 |
| 336 | 3488 | AURKB | 0.996508 |
| 337 | 3130 | FRAP1 | 0.996898 |
| 338 | 3691 | NM_024046 | 0.997251 |
| 339 | 3683 | NM_003138 | 0.998179 |
| 340 | 3569 | RASGRP2 | 1.000543 |
| 341 | 2920 | EIF2AK3 | 1.005703 |
| 342 | 3365 | PRIM1 | 1.006112 |
| 343 | 3462 | TGFBR2 | 1.00726 |
| 344 | 3513 | ROS1 | 1.009016 |
| 345 | 3102 | CKS2 | 1.013052 |
| 346 | 2945 | CDC42BPB | 1.01398 |
| 347 | 3656 | PRKCN | 1.016229 |
| 348 | 3726 | MAPKAPK2 | 1.016458 |
| 349 | 3002 | CRKL | 1.01909 |
| 350 | 3670 | MAP3K10 | 1.01919 |
| 351 | 3767 | FZD3 | 1.019811 |
| 352 | 3645 | CASK | 1.020319 |
| 353 | 3707 | TXK | 1.022666 |
| 354 | 3455 | MAP2K4 | 1.023218 |
| 355 | 3372 | TUBA8 | 1.025814 |
| 356 | 3540 | PPP2CB | 1.027826 |
| 357 | 3690 | PRKAA2 | 1.029902 |
| 358 | 3307 | CDC6 | 1.03012 |
| 359 | 3495 | FGFR2 | 1.032093 |
| 360 | 3485 | DHX8 | 1.032492 |
| 361 | 3696 | NM_016281 | 1.038149 |
| 362 | 3716 | ULK1 | 1.039167 |
| 363 | 3265 | RAF1 | 1.039442 |
| 364 | 3161 | WT1 | 1.039655 |
| 365 | 3215 | MAD2L1 | 1.039783 |
| 366 | 3415 | HSPCA | 1.040186 |
| 367 | 3127 | MAPK1 | 1.040277 |
| 368 | 3686 | MAP3K8 | 1.040303 |
| 369 | 3490 | ERBB3 | 1.040323 |
| 370 | 3441 | PRKCI | 1.042373 |
| 371 | 3115 | MDM2 | 1.04276 |
| 372 | 3264 | CDK3 | 1.044285 |
| 373 | 3147 | CDKN2A | 1.045872 |
| 374 | 3568 | PLD1 | 1.048696 |
| 375 | 3559 | RAP1GDS1 | 1.05 |
| 376 | 2928 | IRAK1 | 1.050577 |
| 377 | 3197 | ARHB | 1.052064 |
| 378 | 3785 | GRB2 | 1.052525 |
| 379 | 3248 | JAK2 | 1.053539 |
| 380 | 3199 | NF2 | 1.053654 |
| 381 | 2992 | PRKR | 1.055468 |
| 382 | 3516 | ARHA | 1.058051 |
| 383 | 3449 | TBK1 | 1.059537 |
| 384 | 2953 | MAPK11 | 1.059656 |
| 385 | 3164 | MYCL1 | 1.060646 |
| 386 | 3745 | CAMKK2 | 1.061685 |
| 387 | 3324 | CUL5 | 1.062571 |
| 388 | 3243 | NM_004203 | 1.062998 |
| 389 | 3187 | WNT7B | 1.063935 |
| 390 | 3459 | EGFR | 1.066553 |
| 391 | 3239 | CDK6 | 1.067257 |
| 392 | 3170 | BLM | 1.068402 |
| 393 | 2943 | DYRK2 | 1.06862 |
| 394 | 3320 | CCND3 | 1.070018 |
| 395 | 3369 | NM_007027 | 1.071887 |
| 396 | 3624 | CTNNB1 | 1.072588 |
| 397 | 3500 | SGK | 1.074011 |
| 398 | 3101 | MAPK14 | 1.074871 |
| 399 | 3408 | PIN1 | 1.075614 |
| 400 | 2924 | STK25 | 1.076046 |
| 401 | 3548 | RAC1 | 1.07851 |
| 402 | 3676 | MAP4K1 | 1.079121 |
| 403 | 3698 | ADRBK2 | 1.079569 |
| 404 | 3301 | CCNB1 | 1.080243 |
| 405 | 2925 | FYN | 1.081081 |
| 406 | 3565 | RAB2 | 1.081968 |
| 407 | 2977 | RIPK3 | 1.082037 |
| 408 | 3810 | AI278633 | 1.084388 |
| 409 | 3796 | ARHGEF6 | 1.084848 |
| 410 | 3116 | KIF5A | 1.086755 |
| 411 | 3590 | ARHGEF2 | 1.088083 |
| 412 | 3679 | CLK2 | 1.088737 |
| 413 | 3119 | CDKN1B | 1.089067 |
| 414 | 3367 | DHFR | 1.092319 |
| 415 | 3797 | ARHGEF9 | 1.092391 |
| 416 | 3405 | HDAC8 | 1.096856 |
| 417 | 2957 | TYK2 | 1.099156 |
| 418 | 3091 | KIFC3 | 1.10008 |
| 419 | 3546 | INPP5D | 1.102828 |
| 420 | 3227 | NUMA1 | 1.104478 |
| 421 | 3181 | ST5 | 1.104782 |
| 422 | 3807 | SPAG5 | 1.105317 |
| 423 | 3090 | KIF3C | 1.10597 |
| 424 | 3343 | CENPJ | 1.107383 |
| 425 | 3245 | AXL | 1.108766 |
| 426 | 3097 | KIF20A | 1.108842 |
| 427 | 3360 | RRM2 | 1.109827 |
| 428 | 3349 | IMPDH1 | 1.111043 |
| 429 | 3474 | CSNK2A1 | 1.111842 |
| 430 | 3616 | FZD1 | 1.11295 |
| 431 | 3620 | AXIN2 | 1.113386 |
| 432 | 2995 | PTK2 | 1.115385 |
| 433 | 3634 | BTRC | 1.117674 |
| 434 | 3504 | PIK3CB | 1.118194 |
| 435 | 3561 | FOS | 1.118649 |
| 436 | 3618 | DVL2 | 1.12 |
| 437 | 3537 | EIF4EBP1 | 1.121316 |
| 438 | 3550 | PLCG1 | 1.121971 |
| 439 | 3443 | EPS8 | 1.122744 |
| 440 | 3370 | AR | 1.123767 |
| 441 | 3543 | PDK2 | 1.12548 |
| 442 | 3122 | ATSV | 1.127371 |
| 443 | 3167 | S100A2 | 1.127907 |
| 444 | 3596 | SHMT1 | 1.128114 |
| 445 | 3811 | NM_152524 | 1.129555 |
| 446 | 3779 | CTNNA1 | 1.129565 |
| 447 | 3312 | CUL3 | 1.133047 |
| 448 | 2963 | MAP3K11 | 1.133758 |
| 449 | 2942 | TTN | 1.133889 |
| 450 | 3790 | ERBB3 | 1.135274 |
| 451 | 3094 | KIF3A | 1.13729 |
| 452 | 3545 | IRS2 | 1.139283 |
| 453 | 3305 | CDC2L5 | 1.140753 |
| 454 | 3748 | NM_016507 | 1.140961 |
| 455 | 3614 | CTNND2 | 1.141748 |
| 456 | 3437 | FGFR1 | 1.143284 |
| 457 | 3389 | NM_052963 | 1.145845 |
| 458 | 3213 | NM_016238 | 1.145939 |
| 459 | 3533 | HES7 | 1.148773 |
| 460 | 3321 | CDKN3 | 1.152745 |
| 461 | 3711 | LIMK1 | 1.153559 |
| 462 | 3503 | EGR1 | 1.156344 |
| 463 | 3701 | STK10 | 1.160598 |
| 464 | 3608 | MAP3K7IP1 | 1.161191 |
| 465 | 3730 | TESK1 | 1.162946 |
| 466 | 3156 | MSH2 | 1.163507 |
| 467 | 3571 | VAV1 | 1.164063 |
| 468 | 3668 | DAPK3 | 1.165365 |
| 469 | 3677 | HCK | 1.166105 |
| 470 | 3708 | RPS6KC1 | 1.166667 |
| 471 | 3110 | KIF13A | 1.167294 |
| 472 | 3185 | VCAM1 | 1.170254 |
| 473 | 3837 | PRKAA1 | 1.171443 |
| 474 | 3514 | HRAS | 1.171476 |
| 475 | 3371 | NM_006087 | 1.175311 |
| 476 | 3420 | NM_014109 | 1.176378 |
| 477 | 3669 | NTRK3 | 1.17801 |
| 478 | 2939 | TLK1 | 1.179137 |
| 479 | 3654 | VRK2 | 1.180868 |
| 480 | 3636 | STAT2 | 1.181562 |
| 481 | 3506 | XM_095827 | 1.18376 |
| 482 | 3728 | TIE | 1.184901 |
| 483 | 3496 | EIF4EBP2 | 1.188138 |
| 484 | 2994 | MATK | 1.188439 |
| 485 | 3353 | PGR | 1.188925 |
| 486 | 3771 | PIK3CA | 1.191131 |
| 487 | 3111 | KIF5B | 1.191167 |
| 488 | 3396 | HDAC10 | 1.192015 |
| 489 | 3330 | CDK9 | 1.194303 |
| 490 | 3705 | NM_012119 | 1.195395 |
| 491 | 3339 | CCNB2 | 1.195402 |
| 492 | 3005 | MERTK | 1.196303 |
| 493 | 3220 | ANAPC11 | 1.1994 |
| 494 | 3507 | NM_145754 | 1.199485 |
| 495 | 3418 | CENPA | 1.199564 |
| 496 | 3492 | PRKCQ | 1.199597 |
| 497 | 3499 | GRB2 | 1.204124 |
| 498 | 3667 | NM_016542 | 1.204923 |
| 499 | 3084 | KIF14 | 1.207333 |
| 500 | 3317 | CCNI | 1.208734 |
| 501 | 3457 | BAD | 1.208929 |
| 502 | 3819 | TACC3 | 1.209677 |
| 503 | 3377 | NM_006088 | 1.210588 |
| 504 | 3472 | MAP3K5 | 1.210677 |
| 505 | 2922 | NM_004783 | 1.211356 |
| 506 | 3453 | MAP2K2 | 1.212321 |
| 507 | 3724 | EPHA7 | 1.213738 |
| 508 | 3260 | STK11 | 1.214815 |
| 509 | 3675 | ADRBK1 | 1.215503 |
| 510 | 3379 | NM_032525 | 1.223512 |
| 511 | 2956 | PRKCL2 | 1.223938 |
| 512 | 3666 | PAK6 | 1.229403 |
| 513 | 3216 | CDC20 | 1.231173 |
| 514 | 3672 | SYK | 1.231714 |
| 515 | 3555 | RASA1 | 1.236402 |
| 516 | 3354 | RRM1 | 1.237695 |
| 517 | 3153 | RB1 | 1.237825 |
| 518 | 3253 | PRKCA | 1.239404 |
| 519 | 3146 | KIF21A | 1.240245 |
| 520 | 3756 | CDK5RAP2 | 1.242775 |
| 521 | 3721 | ANKRD3 | 1.245185 |
| 522 | 3224 | FBXO5 | 1.24973 |
| 523 | 3607 | TLE1 | 1.250329 |
| 524 | 2981 | ALK | 1.252514 |
| 525 | 2978 | AB067470 | 1.252713 |
| 526 | 3440 | FGFR3 | 1.253731 |
| 527 | 3578 | RREB1 | 1.256567 |
| 528 | 3393 | HDAC2 | 1.258824 |
| 529 | 3520 | NRAS | 1.263715 |
| 530 | 3190 | WNT4 | 1.265328 |
| 531 | 3463 | TEC | 1.265973 |
| 532 | 3621 | CTNNA2 | 1.26658 |
| 533 | 3425 | DLG7 | 1.267399 |
| 534 | 3311 | CDK10 | 1.269347 |
| 535 | 3567 | SHC1 | 1.270057 |
| 536 | 3753 | CDK5 | 1.276163 |
| 537 | 2989 | ACVR1B | 1.276215 |
| 538 | 3692 | AB007941 | 1.27931 |
| 539 | 3244 | PRKCZ | 1.279368 |
| 540 | 3092 | KIF12 | 1.279896 |
| 541 | 3487 | MAP2K3 | 1.280835 |
| 542 | 3813 | ANLN | 1.282313 |
| 543 | 3198 | ICAM1 | 1.285429 |
| 544 | 3697 | CAMK2G | 1.286036 |
| 545 | 3735 | PRKACB | 1.286694 |
| 546 | 3100 | GSK3B | 1.289078 |
| 547 | 3431 | NM_018454 | 1.289806 |
| 548 | 3615 | FZD2 | 1.292222 |
| 549 | 2947 | NM_007064 | 1.29381 |
| 550 | 3340 | CENPH | 1.293935 |
| 551 | 3172 | PLAU | 1.297571 |
| 552 | 3160 | TACSTD1 | 1.297585 |
| 553 | 3212 | NM_022662 | 1.301215 |
| 554 | 3098 | CENPE | 1.305802 |
| 555 | 3626 | DVL3 | 1.306682 |
| 556 | 3830 | NM_013296 | 1.307494 |
| 557 | 3713 | PRKG2 | 1.307933 |
| 558 | 3768 | ARAF1 | 1.308011 |
| 559 | 3493 | MAPK9 | 1.308449 |
| 560 | 3108 | KIF22 | 1.308726 |
| 561 | 3169 | NME1 | 1.310985 |
| 562 | 3125 | NM_031217 | 1.311267 |
| 563 | 3375 | AHCY | 1.311852 |
| 564 | 3583 | JUNB | 1.31241 |
| 565 | 3458 | ARAF1 | 1.315519 |
| 566 | 3612 | TCF1 | 1.316285 |
| 567 | 3294 | CCNF | 1.317748 |
| 568 | 3338 | CUL4A | 1.318527 |
| 569 | 3649 | CAMK2B | 1.322337 |
| 570 | 3576 | GRAP | 1.322985 |
| 571 | 3527 | DTX2 | 1.33023 |
| 572 | 3145 | C20orf23 | 1.334687 |
| 573 | 3180 | CD44 | 1.335574 |
| 574 | 3758 | RAD51L1 | 1.335901 |
| 575 | 3165 | FGF2 | 1.336082 |
| 576 | 3828 | KIF20A | 1.337004 |
| 577 | 3553 | CKS1B | 1.339383 |
| 578 | 3089 | KIFC1 | 1.341566 |
| 579 | 3442 | ERBB4 | 1.345118 |
| 580 | 3554 | PIK3R3 | 1.347147 |
| 581 | 3613 | DVL1 | 1.347505 |
| 582 | 2985 | MKNK1 | 1.347934 |
| 583 | 3117 | ATM | 1.348967 |
| 584 | 3424 | EZH2 | 1.352941 |
| 585 | 3695 | PRKAA1 | 1.355145 |
| 586 | 3446 | PRKCG | 1.355556 |
| 587 | 3194 | RARB | 1.359932 |
| 588 | 3644 | EPHB1 | 1.36061 |
| 589 | 3700 | AB037782 | 1.361005 |
| 590 | 3599 | DTYMK | 1.361789 |
| 591 | 3729 | RYK | 1.361997 |
| 592 | 3114 | PIK3CD | 1.362808 |
| 593 | 3821 | ASPM | 1.363705 |
| 594 | 3373 | TOP2A | 1.363708 |
| 595 | 3563 | RAB3A | 1.365615 |
| 596 | 3764 | NOTCH4 | 1.36911 |
| 597 | 3628 | CTNNBL1 | 1.3702 |
| 598 | 3823 | NM_017779 | 1.372126 |
| 599 | 3715 | CDC42BPA | 1.372256 |
| 600 | 3562 | RASD1 | 1.372563 |
| 601 | 3784 | MAPK8 | 1.376577 |
| 602 | 3574 | SH3KBP1 | 1.384674 |
| 603 | 3594 | RAP2A | 1.393939 |
| 604 | 3662 | LCK | 1.3981 |
| 605 | 3787 | FZD4 | 1.399749 |
| 606 | 3316 | CCNA1 | 1.404295 |
| 607 | 3684 | STK38L | 1.406161 |
| 608 | 3610 | LEF1 | 1.407463 |
| 609 | 3390 | NM_080925 | 1.407563 |
| 610 | 3152 | APC | 1.414678 |
| 611 | 3149 | TP53 | 1.420044 |
| 612 | 3238 | MAP3K3 | 1.420428 |
| 613 | 3109 | KIF1C | 1.420608 |
| 614 | 3325 | CDKN1C | 1.42522 |
| 615 | 3314 | CCNG1 | 1.426516 |
| 616 | 3825 | NM_152562 | 1.428805 |
| 617 | 3588 | RHEB | 1.435039 |
| 618 | 3736 | PTK7 | 1.440171 |
| 619 | 3118 | NM_032559 | 1.440252 |
| 620 | 3521 | MET | 1.440418 |
| 621 | 3096 | AKT1 | 1.440951 |
| 622 | 3361 | IMPDH2 | 1.442308 |
| 623 | 3582 | GRAP2 | 1.444349 |
| 624 | 3584 | RASAL2 | 1.450119 |
| 625 | 3801 | PSEN1 | 1.466292 |
| 626 | 3803 | MPHOSPH1 | 1.470276 |
| 627 | 2938 | ALS2CR7 | 1.471357 |
| 628 | 3106 | KIF9 | 1.47493 |
| 629 | 3313 | CCNG2 | 1.48267 |
| 630 | 3792 | ARHGEF1 | 1.48329 |
| 631 | 3210 | NM_013366 | 1.48366 |
| 632 | 3820 | NM_018410 | 1.483709 |
| 633 | 2932 | MAPKAPK3 | 1.488 |
| 634 | 3747 | GSK3A | 1.491773 |
| 635 | 2962 | MAP4K2 | 1.495448 |
| 636 | 3699 | NM_032844 | 1.502812 |
| 637 | 3189 | MYB | 1.504618 |
| 638 | 3629 | AXIN1 | 1.505556 |
| 639 | 2941 | DYRK3 | 1.505717 |
| 640 | 3818 | AI338451 | 1.511194 |
| 641 | 2919 | OSR1 | 1.512906 |
| 642 | 3140 | XM_089006 | 1.518548 |
| 643 | 3229 | PRKCL1 | 1.525203 |
| 644 | 3510 | CDK4 | 1.529837 |
| 645 | 3319 | CCND1 | 1.531034 |
| 646 | 3159 | RET | 1.536506 |
| 647 | 3242 | PRKCB1 | 1.540024 |
| 648 | 3519 | NTRK1 | 1.547773 |
| 649 | 3808 | CKAP2 | 1.554545 |
| 650 | 2988 | FRK | 1.557214 |
| 651 | 2944 | MARK1 | 1.557763 |
| 652 | 2971 | DAPK2 | 1.55938 |
| 653 | 3299 | CUL1 | 1.560841 |
| 654 | 3660 | DMPK | 1.5625 |
| 655 | 3515 | PDGFRB | 1.562977 |
| 656 | 3522 | KRAS2 | 1.564353 |
| 657 | 3004 | MAP3K1 | 1.570175 |
| 658 | 3395 | HDAC5 | 1.571159 |
| 659 | 3468 | ABL2 | 1.571225 |
| 660 | 3529 | DTX1 | 1.57276 |
| 661 | 3329 | CDC42 | 1.580386 |
| 662 | 3704 | ACVR2B | 1.58046 |
| 663 | 3827 | NM_018123 | 1.581315 |
| 664 | 3456 | FLT1 | 1.583826 |
| 665 | 3310 | CDC34 | 1.585818 |
| 666 | 3331 | CDC25B | 1.585938 |
| 667 | 3368 | TOP3A | 1.58728 |
| 668 | 3126 | KIF3B | 1.588728 |
| 669 | 3780 | MCM3 | 1.590296 |
| 670 | 3128 | AKT2 | 1.592696 |
| 671 | 3598 | PCNA | 1.59319 |
| 672 | 3535 | SKP2 | 1.593333 |
| 673 | 2955 | PAK1 | 1.59552 |
| 674 | 3234 | CDK2 | 1.596033 |
| 675 | 3138 | KIF17 | 1.604846 |
| 676 | 3632 | WISP1 | 1.607319 |
| 677 | 3611 | CTNNAL1 | 1.611386 |
| 678 | 3300 | CDC14B | 1.611486 |
| 679 | 3511 | XM_168069 | 1.614698 |
| 680 | 3144 | KIF4B | 1.619674 |
| 681 | 3627 | CTNNA1 | 1.620915 |
| 682 | 3337 | CDKN1A | 1.626582 |
| 683 | 3202 | MCC | 1.627957 |
| 684 | 3143 | NM_017596 | 1.628521 |
| 685 | 3186 | ETS1 | 1.635593 |
| 686 | 3432 | PRC1 | 1.637647 |
| 687 | 3556 | RAP1A | 1.638173 |
| 688 | 3335 | CDK5R2 | 1.656172 |
| 689 | 2933 | MAP4K5 | 1.656522 |
| 690 | 2927 | MAPK13 | 1.659401 |
| 691 | 2973 | NEK1 | 1.664311 |
| 692 | 3538 | NFKB2 | 1.667808 |
| 693 | 3602 | MCM3 | 1.678819 |
| 694 | 3603 | POLS | 1.678937 |
| 695 | 3630 | WISP3 | 1.679045 |
| 696 | 3447 | PRKCM | 1.680152 |
| 697 | 3402 | HDAC4 | 1.68123 |
| 698 | 3133 | XM_168069 | 1.681935 |
| 699 | 3428 | ECT2 | 1.690096 |
| 700 | 3720 | AB002301 | 1.691718 |
| 701 | 3793 | MAPRE1 | 1.693966 |
| 702 | 3681 | SRPK1 | 1.700611 |
| 703 | 3817 | NM_019013 | 1.702326 |
| 704 | 3136 | XM_066649 | 1.708388 |
| 705 | 3355 | GUK1 | 1.710938 |
| 706 | 3087 | PTEN | 1.716866 |
| 707 | 3579 | PDZGEF2 | 1.717714 |
| 708 | 3168 | DCC | 1.719083 |
| 709 | 3151 | MLH1 | 1.72077 |
| 710 | 3217 | NM_014885 | 1.722045 |
| 711 | 3191 | WNT2 | 1.728016 |
| 712 | 3765 | CREBBP | 1.72973 |
| 713 | 3655 | CAMK2D | 1.733773 |
| 714 | 3407 | HDAC9 | 1.748784 |
| 715 | 3255 | CDK7 | 1.75 |
| 716 | 3295 | CDK2AP1 | 1.75 |
| 717 | 3192 | WNT1 | 1.751208 |
| 718 | 3333 | CCNT2 | 1.761104 |
| 719 | 3703 | AK024504 | 1.764425 |
| 720 | 3760 | CDKL5 | 1.769444 |
| 721 | 2948 | MYO3A | 1.769759 |
| 722 | 3800 | CHFR | 1.772809 |
| 723 | 3544 | IRS1 | 1.776668 |
| 724 | 3235 | CHEK1 | 1.776886 |
| 725 | 3137 | KIF26A | 1.782366 |
| 726 | 3673 | DDR1 | 1.792507 |
| 727 | 3336 | CDC37 | 1.807985 |
| 728 | 3725 | EPHA4 | 1.820076 |
| 729 | 3404 | PPARG | 1.822581 |
| 730 | 3604 | TK2 | 1.82846 |
| 731 | 3738 | PRKWNK3 | 1.836245 |
| 732 | 3141 | NM_145754 | 1.843889 |
| 733 | 3451 | MAP3K4 | 1.855556 |
| 734 | 3417 | HDAC3 | 1.857143 |
| 735 | 3508 | KIF25 | 1.871592 |
| 736 | 3575 | LATS2 | 1.879574 |
| 737 | 3761 | WT1 | 1.88089 |
| 738 | 3723 | NM_018401 | 1.88722 |
| 739 | 3719 | BMPR2 | 1.890545 |
| 740 | 3204 | CDC16 | 1.892826 |
| 741 | 3467 | MAP2K7 | 1.894459 |
| 742 | 2986 | ACVR2 | 1.896882 |
| 743 | 3218 | CDC23 | 1.904255 |
| 744 | 3791 | NM_005200 | 1.913043 |
| 745 | 3804 | NM_024322 | 1.920139 |
| 746 | 3558 | RALA | 1.92029 |
| 747 | 3824 | MAPRE3 | 1.940871 |
| 748 | 3622 | FZD9 | 1.988166 |
| 749 | 3205 | NM_139286 | 1.997054 |
| 750 | 3221 | TOP3B | 1.997534 |
| 751 | 3794 | WASL | 1.998403 |
| 752 | 3637 | STAT4 | 2.005199 |
| 753 | 3834 | CHEK1 | 2.01625 |
| 754 | 3400 | BCL2 | 2.045028 |
| 755 | 3223 | NM_016263 | 2.045139 |
| 756 | 3358 | TOP2B | 2.050562 |
| 757 | 3512 | TGFBR1 | 2.062016 |
| 758 | 3259 | MAPK8 | 2.064081 |
| 759 | 3742 | RHOK | 2.075949 |
| 760 | 2946 | NM_017719 | 2.078131 |
| 761 | 3406 | TERT | 2.10274 |
| 762 | 3206 | ANAPC5 | 2.159615 |
| 763 | 3531 | NM_021170 | 2.163086 |
| 764 | 3008 | SGK2 | 2.1766 |
| 765 | 3706 | C20orf97 | 2.1875 |
| 766 | 3254 | CSF1R | 2.196822 |
| 767 | 3439 | EGR2 | 2.213333 |
| 768 | 2970 | AATK | 2.235211 |
| 769 | 3528 | TCF3 | 2.273649 |
| 770 | 3327 | CDC45L | 2.288265 |
| 771 | 3551 | STAT3 | 2.29125 |
| 772 | 3001 | PRKY | 2.313131 |
| 773 | 3734 | BMPR1B | 2.330839 |
| 774 | 3095 | KIF2C | 2.336785 |
| 775 | 3222 | PTTG1 | 2.347826 |
| 776 | 3532 | NM_019089 | 2.352437 |
| 777 | 3547 | FOXO1A | 2.352444 |
| 778 | 3671 | STK4 | 2.362408 |
| 779 | 3781 | SRC | 2.37859 |
| 780 | 3789 | ELK1 | 2.394828 |
| 781 | 3247 | NM_018492 | 2.480851 |
| 782 | 3586 | RASA2 | 2.506796 |
| 783 | 3727 | GPRK6 | 2.553987 |
| 784 | 3689 | BLK | 2.584588 |
| 785 | 3777 | ABL1 | 2.615226 |
| 786 | 3399 | HSPCB | 2.632207 |
| 787 | 2958 | PRKACA | 2.635514 |
| 788 | 3304 | CCNE2 | 2.677656 |
| 789 | 3617 | CTNNBIP1 | 2.698292 |
| 790 | 3225 | NM_013367 | 2.714286 |
| 791 | 3619 | FRAT1 | 2.728111 |
| 792 | 3121 | PIK3C2A | 2.828125 |
| 793 | 3816 | NM_017769 | 2.847273 |
| 794 | 3134 | XM_170783 | 2.923286 |
| 795 | 3737 | NM_016457 | 2.940451 |
| 796 | 3135 | XM_064050 | 3.063002 |
| 797 | 3129 | STK6 | 3.146434 |
| 798 | 3564 | RALBP1 | 3.170605 |
| 799 | 3580 | ELK1 | 3.356401 |
| 800 | 3157 | NF1 | 3.402273 |
| 801 | 3638 | STAT5A | 3.754386 |
| 802 | 3241 | WEE1 | 3.801887 |
| 803 | 3718 | PTK6 | 4.317857 |
| 804 | 3712 | RPS6KA6 | 5.356624 |
| 805 | 3158 | BRCA1 | 5.821429 |
| 806 | 3642 | EPHB3 | 6.43 |
| 807 | 3150 | BRCA2 | 14.13136 |

TABLE IIC
Average fold sensitization by doxorubicin

| | | | ave of 3 |
|---|---|---|---|
| Gene ID | BioID | Gene | screens |
| 1 | 2514 | PLK | 0.094489 |
| 2 | 3099 | PLK | 0.195626 |
| 3 | 3099 | PLK | 0.211482 |
| 4 | 3099 | PLK | 0.211747 |
| 5 | 3099 | PLK | 0.219626 |
| 6 | 3099 | PLK | 0.227603 |
| 7 | 3099 | PLK | 0.235482 |
| 8 | 3099 | PLK | 0.235683 |
| 9 | 3099 | PLK | 0.235747 |
| 10 | 3099 | PLK | 0.251539 |
| 11 | 3099 | PLK | 0.251603 |
| 12 | 3099 | PLK | 0.259683 |
| 13 | 3099 | PLK | 0.275539 |
| 14 | 3099 | PLK | 0.282503 |
| 15 | 3099 | PLK | 0.298359 |
| 16 | 3099 | PLK | 0.298624 |
| 17 | 3099 | PLK | 0.31448 |
| 18 | 3099 | PLK | 0.32256 |
| 19 | 3534 | PLK | 0.330807 |
| 20 | 3099 | PLK | 0.338416 |
| 21 | 3099 | PLK | 0.395491 |
| 22 | 3099 | PLK | 0.411612 |
| 23 | 3006 | PLK | 0.415454 |
| 24 | 3099 | PLK | 0.419491 |
| 25 | 3099 | PLK | 0.435548 |
| 26 | 3099 | PLK | 0.435612 |
| 27 | 3433 | PLK | 0.435845 |
| 28 | 3391 | PLK | 0.440842 |
| 29 | 3099 | PLK | 0.459548 |
| 30 | 3099 | PLK | 0.482368 |
| 31 | 3099 | PLK | 0.498489 |
| 32 | 3322 | CCNA2 | 0.512614 |
| 33 | 3099 | PLK | 0.522425 |
| 34 | 3805 | C20orf1 | 0.562328 |
| 35 | 3423 | | 0.613084 |
| 36 | 3600 | RRM2B | 0.659243 |
| 37 | 3305 | CDC2L5 | 0.68014 |
| 38 | 3542 | PPP2CA | 0.695506 |
| 39 | 3266 | PLK | 0.696721 |
| 40 | 3228 | CDC27 | 0.70157 |
| 41 | 3464 | INSR | 0.70706 |
| 42 | 3326 | CCT7 | 0.724986 |
| 43 | 3740 | STK35 | 0.754807 |
| 44 | 3731 | CSNK1E | 0.765738 |
| 45 | 3416 | IKBKE | 0.773235 |
| 46 | 3293 | CDC25A | 0.77957 |
| 47 | 3309 | CCND2 | 0.791487 |
| 48 | 3350 | ADA | 0.800034 |
| 49 | 3812 | CDCA8 | 0.815766 |
| 50 | 3354 | RRM1 | 0.817751 |
| 51 | 3446 | PRKCG | 0.822809 |
| 52 | 3648 | CLK1 | 0.824307 |
| 53 | 3509 | KIF21A | 0.826427 |
| 54 | 3526 | HES6 | 0.826991 |
| 55 | 3250 | CHEK2 | 0.828202 |
| 56 | 3262 | LATS1 | 0.82944 |
| 57 | 3359 | TUBA1 | 0.839308 |
| 58 | 3344 | CDKL1 | 0.840425 |
| 59 | 2984 | EPHB6 | 0.846685 |
| 60 | 3702 | MAP3K13 | 0.84685 |
| 61 | 3838 | PRKAA2 | 0.853115 |
| 62 | 3422 | SMC4L1 | 0.854651 |
| 63 | 3332 | CDC5L | 0.85491 |
| 64 | 3750 | CAMK2A | 0.857171 |
| 65 | 3686 | MAP3K8 | 0.8599 |
| 66 | 3226 | RBX1 | 0.862335 |
| 67 | 3438 | DTR | 0.863218 |
| 68 | 3318 | CUL2 | 0.863485 |
| 69 | 3454 | FLT4 | 0.864511 |
| 70 | 3366 | TYMS | 0.866092 |
| 71 | 3444 | NEK3 | 0.866318 |
| 72 | 3397 | BUB3 | 0.867363 |
| 73 | 3007 | MAP3K14 | 0.86906 |
| 74 | 3373 | TOP2A | 0.875387 |
| 75 | 2934 | IRAK2 | 0.875671 |
| 76 | 3188 | PMS2 | 0.876644 |
| 77 | 3461 | KIT | 0.876727 |
| 78 | 3398 | HDAC11 | 0.878587 |
| 79 | 3665 | PAK4 | 0.879213 |
| 80 | 3494 | MAPK4 | 0.879947 |
| 81 | 3303 | CDKN2D | 0.88429 |
| 82 | 2925 | FYN | 0.885569 |
| 83 | 3437 | FGFR1 | 0.889075 |
| 84 | 3219 | CENPC1 | 0.889832 |
| 85 | 3491 | PRKCE | 0.891708 |
| 86 | 3105 | BUB1 | 0.892262 |
| 87 | 3609 | FZD3 | 0.89297 |
| 88 | 3421 | ORC6L | 0.893859 |
| 89 | 3414 | HDAC7A | 0.894925 |
| 90 | 3342 | CCNT1 | 0.89645 |
| 91 | 3193 | FGF3 | 0.897275 |
| 92 | 3203 | ITGA5 | 0.89915 |
| 93 | 3679 | CLK2 | 0.899792 |
| 94 | 3656 | PRKCN | 0.903305 |
| 95 | 3677 | HCK | 0.903727 |
| 96 | 3172 | PLAU | 0.904045 |
| 97 | 2999 | FES | 0.904351 |
| 98 | 3161 | WT1 | 0.907863 |
| 99 | 3230 | MAP2K1 | 0.908157 |
| 100 | 2937 | | 0.910875 |
| 101 | 3502 | PRKCH | 0.913184 |
| 102 | 3317 | CCNI | 0.913695 |
| 103 | 3086 | KIF11 | 0.914508 |
| 104 | 3412 | KIF25 | 0.915671 |
| 105 | 3710 | GPRK2L | 0.917359 |
| 106 | 3585 | GAB1 | 0.91762 |
| 107 | 3807 | SPAG5 | 0.918025 |
| 108 | 3815 | MAPRE2 | 0.919461 |
| 109 | 3646 | | 0.920311 |
| 110 | 3000 | BMX | 0.920926 |
| 111 | 3365 | PRIM1 | 0.922943 |
| 112 | 3574 | SH3KBP1 | 0.924261 |
| 113 | 3485 | DHX8 | 0.924589 |
| 114 | 3527 | DTX2 | 0.92511 |
| 115 | 3378 | | 0.927814 |
| 116 | 3799 | ACTR3 | 0.929286 |
| 117 | 3822 | GTSE1 | 0.929871 |
| 118 | 3100 | GSK3B | 0.932676 |
| 119 | 3206 | ANAPC5 | 0.932816 |
| 120 | 3351 | ESR1 | 0.932858 |
| 121 | 3623 | CTNND1 | 0.932974 |
| 122 | 3601 | POLE | 0.935664 |
| 123 | 3097 | KIF20A | 0.939338 |
| 124 | 2991 | NPR1 | 0.941392 |
| 125 | 2926 | | 0.943073 |
| 126 | 3717 | NTRK2 | 0.94323 |
| 127 | 3162 | MADH2 | 0.953335 |
| 128 | 3783 | KRAS2 | 0.954957 |
| 129 | 3660 | DMPK | 0.955308 |
| 130 | 3236 | PTK2B | 0.955874 |
| 131 | 3088 | KIF13B | 0.960206 |
| 132 | 3774 | CDC45L | 0.961565 |
| 133 | 3540 | PPP2CB | 0.96255 |
| 134 | 3251 | ABL1 | 0.96267 |
| 135 | 3498 | CSNK1A1 | 0.963185 |
| 136 | 3307 | CDC6 | 0.963749 |
| 137 | 3830 | | 0.96419 |
| 138 | 3374 | POLR2A | 0.964327 |
| 139 | 3413 | KIF23 | 0.967774 |
| 140 | 3296 | CDKN2C | 0.967818 |
| 141 | 3132 | KIF23 | 0.96794 |
| 142 | 3708 | RPS6KC1 | 0.969675 |
| 143 | 3445 | BMPR1A | 0.970178 |
| 144 | 3694 | STK38 | 0.970842 |
| 145 | 3566 | JUN | 0.971389 |
| 146 | 3140 | | 0.97186 |
| 147 | 3571 | VAV1 | 0.972374 |
| 148 | 2993 | SRMS | 0.972957 |
| 149 | 3268 | CDC25C | 0.973198 |
| 150 | 3835 | CHEK2 | 0.973353 |
| 151 | 3557 | JUND | 0.973868 |
| 152 | 3195 | CDH1 | 0.973895 |
| 153 | 3375 | AHCY | 0.974215 |
| 154 | 3163 | THRA | 0.976052 |
| 155 | 3164 | MYCL1 | 0.979364 |
| 156 | 3798 | ACTR2 | 0.980521 |
| 157 | 3392 | BIRC5 | 0.980792 |
| 158 | 3196 | ARHI | 0.980973 |
| 159 | 3536 | PIK3C3 | 0.981403 |
| 160 | 2950 | NEK6 | 0.981709 |
| 161 | 3773 | WNT2 | 0.982648 |
| 162 | 3776 | NOTCH2 | 0.983584 |
| 163 | 3814 | HMMR | 0.983597 |
| 164 | 3234 | CDK2 | 0.983724 |
| 165 | 2982 | CDC2L2 | 0.984121 |
| 166 | 3826 | | 0.985249 |
| 167 | 2953 | MAPK11 | 0.987788 |
| 168 | 3403 | AURKC | 0.988679 |
| 169 | 3586 | RASA2 | 0.989648 |
| 170 | 3503 | EGR1 | 0.991443 |
| 171 | 3166 | PMS1 | 0.99314 |
| 172 | 3394 | HDAC6 | 0.994139 |
| 173 | 3652 | PDGFRA | 0.994658 |
| 174 | 3625 | CTBP2 | 0.994928 |
| 175 | 3294 | CCNF | 0.995133 |
| 176 | 3260 | STK11 | 0.998488 |
| 177 | 2968 | STK17B | 0.998826 |
| 178 | 3703 | | 0.999818 |
| 179 | 3577 | RAB2L | 1.00021 |
| 180 | 3184 | TSG101 | 1.00109 |
| 181 | 2927 | MAPK13 | 1.001159 |
| 182 | 3116 | KIF5A | 1.002239 |
| 183 | 3496 | EIF4EBP2 | 1.005451 |
| 184 | 3741 | RPS6KB2 | 1.00589 |
| 185 | 3298 | CCNE1 | 1.005922 |
| 186 | 2990 | | 1.006496 |
| 187 | 3142 | KIF25 | 1.006522 |
| 188 | 3218 | CDC23 | 1.009586 |
| 189 | 3517 | MYC | 1.010689 |
| 190 | 2997 | MST1R | 1.011122 |
| 191 | 3003 | FER | 1.012506 |
| 192 | 3700 | | 1.013542 |
| 193 | 3470 | RPS6KA1 | 1.013802 |
| 194 | 3439 | EGR2 | 1.013847 |
| 195 | 3429 | MCM6 | 1.014653 |
| 196 | 3372 | TUBA8 | 1.017048 |
| 197 | 3556 | RAP1A | 1.017133 |
| 198 | 3155 | ATR | 1.017435 |
| 199 | 3649 | CAMK2B | 1.017461 |
| 200 | 3501 | RPS6KA2 | 1.018616 |
| 201 | 3336 | CDC37 | 1.019161 |
| 202 | 2928 | IRAK1 | 1.021732 |
| 203 | 3733 | MYLK2 | 1.021742 |
| 204 | 2960 | LYN | 1.022112 |
| 205 | 3301 | CCNB1 | 1.022891 |
| 206 | 3743 | RAGE | 1.023372 |
| 207 | 3525 | NOTCH4 | 1.02341 |
| 208 | 3767 | FZD3 | 1.023646 |
| 209 | 2954 | ROCK2 | 1.02397 |
| 210 | 3475 | EPHB4 | 1.024709 |
| 211 | 3635 | STAT1 | 1.026128 |
| 212 | 3746 | HUNK | 1.026176 |
| 213 | 2977 | RIPK3 | 1.0272 |
| 214 | 3573 | | 1.028343 |
| 215 | 3751 | BCR | 1.028418 |
| 216 | 3112 | KNSL7 | 1.029109 |
| 217 | 3488 | AURKB | 1.029885 |
| 218 | 3356 | TUBG1 | 1.029908 |
| 219 | 3364 | HPRT1 | 1.030247 |
| 220 | 3465 | EPHA1 | 1.032043 |
| 221 | 3828 | KIF20A | 1.032108 |
| 222 | 3434 | DDX6 | 1.03425 |
| 223 | 3143 | | 1.03439 |
| 224 | 3212 | | 1.034473 |
| 225 | 3725 | EPHA4 | 1.034871 |
| 226 | 3473 | DAPK1 | 1.035466 |
| 227 | 3581 | RASGRP1 | 1.036407 |
| 228 | 3357 | PRIM2A | 1.036773 |
| 229 | 3469 | AAK1 | 1.037538 |
| 230 | 3171 | VHL | 1.038422 |
| 231 | 3123 | AKT3 | 1.039278 |
| 232 | 3572 | RASA3 | 1.04084 |
| 233 | 3615 | FZD2 | 1.042378 |
| 234 | 3658 | STK18 | 1.043083 |
| 235 | 3261 | ERBB2 | 1.044345 |
| 236 | 3220 | ANAPC11 | 1.0449 |
| 237 | 3639 | STAT6 | 1.045395 |
| 238 | 2959 | PIM2 | 1.048207 |
| 239 | 2935 | MAPK6 | 1.050943 |
| 240 | 3752 | CCNB3 | 1.051148 |
| 241 | 3431 | | 1.05315 |
| 242 | 3101 | MAPK14 | 1.054104 |
| 243 | 3462 | TGFBR2 | 1.056272 |
| 244 | 3319 | CCND1 | 1.057299 |
| 245 | 3592 | SOS2 | 1.058842 |
| 246 | 3655 | CAMK2D | 1.061571 |
| 247 | 3513 | ROS1 | 1.062804 |
| 248 | 3297 | CCT2 | 1.064889 |
| 249 | 3549 | PIK3R2 | 1.066314 |
| 250 | 2998 | MAPK7 | 1.066798 |
| 251 | 3334 | CUL4B | 1.066807 |
| 252 | 3381 | POLR2B | 1.068615 |
| 253 | 3633 | CTBP1 | 1.069269 |
| 254 | 3678 | PFTK1 | 1.07042 |
| 255 | 2987 | RNASEL | 1.072118 |
| 256 | 3256 | CDC2L1 | 1.073967 |
| 257 | 3558 | RALA | 1.074961 |
| 258 | 3749 | | 1.075156 |
| 259 | 3252 | NEK2 | 1.075822 |
| 260 | 2919 | OSR1 | 1.077885 |
| 261 | 3393 | HDAC2 | 1.077906 |
| 262 | 3747 | GSK3A | 1.078401 |
| 263 | 3410 | RPS27 | 1.078517 |
| 264 | 3107 | KIF2 | 1.078686 |
| 265 | 3654 | VRK2 | 1.081195 |
| 266 | 3533 | HES7 | 1.081287 |
| 267 | 2983 | GUCY2C | 1.083605 |
| 268 | 3555 | RASA1 | 1.084083 |
| 269 | 3258 | MOS | 1.084874 |
| 270 | 3180 | CD44 | 1.085294 |
| 271 | 3124 | KIF4A | 1.086165 |
| 272 | 3179 | PDGFB | 1.086599 |
| 273 | 3209 | MAD2L2 | 1.088835 |
| 274 | 3295 | CDK2AP1 | 1.089703 |
| 275 | 3726 | MAPKAPK2 | 1.09032 |
| 276 | 3674 | CSNK1D | 1.090974 |
| 277 | 3616 | FZD1 | 1.091935 |
| 278 | 3452 | JAK1 | 1.092015 |
| 279 | 3823 | | 1.092187 |
| 280 | 3745 | CAMKK2 | 1.092307 |
| 281 | 3149 | TP53 | 1.092755 |
| 282 | 3561 | FOS | 1.092859 |
| 283 | 3836 | TP53 | 1.093641 |
| 284 | 3170 | BLM | 1.094952 |
| 285 | 2930 | ITK | 1.095322 |
| 286 | 3744 | PRKWNK4 | 1.095854 |
| 287 | 3401 | HDAC1 | 1.096531 |
| 288 | 3300 | CDC14B | 1.096651 |
| 289 | 3348 | DCK | 1.096689 |
| 290 | 3405 | HDAC8 | 1.096956 |
| 291 | 3239 | CDK6 | 1.097696 |
| 292 | 3640 | STAT5B | 1.098035 |
| 293 | 2992 | PRKR | 1.098133 |
| 294 | 3548 | RAC1 | 1.09835 |
| 295 | 3306 | CDC14A | 1.098874 |
| 296 | 2943 | DYRK2 | 1.099617 |
| 297 | 3127 | MAPK1 | 1.102044 |
| 298 | 3716 | ULK1 | 1.104258 |
| 299 | 2922 | | 1.106102 |
| 300 | 3160 | TACSTD1 | 1.107397 |
| 301 | 2964 | RIPK2 | 1.109482 |
| 302 | 3634 | BTRC | 1.110007 |
| 303 | 3576 | GRAP | 1.110227 |
| 304 | 3833 | ATR | 1.110614 |
| 305 | 3837 | PRKAA1 | 1.111073 |
| 306 | 2939 | TLK1 | 1.111429 |
| 307 | 3125 | | 1.11143 |
| 308 | 3299 | CUL1 | 1.111864 |
| 309 | 3813 | ANLN | 1.112297 |
| 310 | 3756 | CDK5RAP2 | 1.112508 |
| 311 | 2976 | NEK7 | 1.112602 |
| 312 | 2965 | | 1.11309 |
| 313 | 3784 | MAPK8 | 1.114132 |
| 314 | 3653 | NPR2 | 1.115282 |
| 315 | 3302 | CDK8 | 1.115429 |
| 316 | 3628 | CTNNBL1 | 1.115905 |
| 317 | 3664 | STK17A | 1.115938 |
| 318 | 3504 | PIK3CB | 1.117357 |
| 319 | 3395 | HDAC5 | 1.118952 |
| 320 | 3369 | | 1.119457 |
| 321 | 3243 | | 1.119572 |
| 322 | 3715 | CDC42BPA | 1.119975 |
| 323 | 2924 | STK25 | 1.122952 |
| 324 | 3568 | PLD1 | 1.123878 |
| 325 | 3676 | MAP4K1 | 1.124218 |
| 326 | 3343 | CENPJ | 1.127564 |
| 327 | 3238 | MAP3K3 | 1.127647 |
| 328 | 3424 | EZH2 | 1.127778 |
| 329 | 3418 | CENPA | 1.128399 |
| 330 | 3829 | KIF2C | 1.128457 |
| 331 | 3476 | PRKCD | 1.128572 |
| 332 | 3407 | HDAC9 | 1.129023 |
| 333 | 2951 | | 1.13065 |
| 334 | 3685 | LTK | 1.130723 |
| 335 | 2942 | TTN | 1.131132 |
| 336 | 3085 | KIF5C | 1.133235 |
| 337 | 3367 | DHFR | 1.133721 |
| 338 | 3362 | NR3C1 | 1.134725 |
| 339 | 3400 | BCL2 | 1.134785 |
| 340 | 3800 | CHFR | 1.134967 |
| 341 | 3103 | PIK3CA | 1.135082 |
| 342 | 3711 | LIMK1 | 1.135687 |
| 343 | 3165 | FGF2 | 1.136323 |
| 344 | 3213 | | 1.137328 |
| 345 | 3370 | AR | 1.137843 |
| 346 | 3772 | RPS6KB1 | 1.138023 |
| 347 | 3189 | MYB | 1.138695 |
| 348 | 3631 | WISP2 | 1.138989 |
| 349 | 2945 | CDC42BPB | 1.140434 |
| 350 | 3593 | VAV2 | 1.141048 |
| 351 | 3338 | CUL4A | 1.141509 |
| 352 | 3092 | KIF12 | 1.14183 |
| 353 | 3782 | SOS1 | 1.14272 |
| 354 | 2989 | ACVR1B | 1.143948 |
| 355 | 3808 | CKAP2 | 1.144074 |
| 356 | 3310 | CDC34 | 1.14429 |
| 357 | 3760 | CDKL5 | 1.144621 |
| 358 | 3159 | RET | 1.144761 |
| 359 | 3508 | KIF25 | 1.144865 |
| 360 | 3788 | PRKCE | 1.145993 |
| 361 | 3231 | ILK | 1.146387 |
| 362 | 3471 | MAPK10 | 1.146497 |
| 363 | 3668 | DAPK3 | 1.14781 |
| 364 | 3595 | FEN1 | 1.14853 |
| 365 | 3775 | NOTCH1 | 1.150372 |
| 366 | 3145 | C20orf23 | 1.151785 |
| 367 | 3570 | RALGDS | 1.152146 |
| 368 | 2972 | KSR | 1.152379 |
| 369 | 3441 | PRKCI | 1.152901 |
| 370 | 3737 | | 1.153373 |
| 371 | 3463 | TEC | 1.154814 |
| 372 | 3748 | | 1.155977 |
| 373 | 3816 | | 1.156751 |
| 374 | 3582 | GRAP2 | 1.158058 |
| 375 | 3360 | RRM2 | 1.158514 |
| 376 | 3516 | ARHA | 1.15962 |
| 377 | 3312 | CUL3 | 1.160258 |
| 378 | 3005 | MERTK | 1.160604 |
| 379 | 3456 | FLT1 | 1.160651 |
| 380 | 3567 | SHC1 | 1.161312 |
| 381 | 3647 | YES1 | 1.161861 |
| 382 | 3447 | PRKCM | 1.162427 |
| 383 | 3739 | | 1.163543 |
| 384 | 3181 | ST5 | 1.163581 |
| 385 | 3466 | JAK3 | 1.164099 |
| 386 | 3311 | CDK10 | 1.1651 |
| 387 | 3486 | RPS6KA3 | 1.165517 |
| 388 | 3779 | CTNNA1 | 1.165697 |
| 389 | 3148 | LIG1 | 1.166358 |
| 390 | 3683 | | 1.167226 |
| 391 | 3544 | IRS1 | 1.167527 |
| 392 | 3335 | CDK5R2 | 1.167989 |
| 393 | 3821 | ASPM | 1.167998 |
| 394 | 3108 | KIF22 | 1.168525 |
| 395 | 3168 | DCC | 1.170395 |
| 396 | 3182 | MYCN | 1.172038 |
| 397 | 3119 | CDKN1B | 1.172505 |
| 398 | 3692 | | 1.173629 |
| 399 | 3687 | CAMK4 | 1.17436 |
| 400 | 3420 | | 1.175153 |
| 401 | 3762 | MCC | 1.175576 |
| 402 | 3519 | NTRK1 | 1.175989 |
| 403 | 3257 | IGF1R | 1.176551 |
| 404 | 3769 | PLAU | 1.176774 |
| 405 | 3339 | CCNB2 | 1.177549 |
| 406 | 3682 | DYRK1A | 1.178203 |
| 407 | 3240 | MAPK12 | 1.178713 |
| 408 | 3156 | MSH2 | 1.17907 |
| 409 | 2936 | SGKL | 1.17989 |
| 410 | 2920 | EIF2AK3 | 1.179969 |
| 411 | 3670 | MAP3K10 | 1.180357 |
| 412 | 3207 | MAD1L1 | 1.181963 |
| 413 | 3630 | WISP3 | 1.182009 |
| 414 | 3153 | RB1 | 1.183084 |
| 415 | 3632 | WISP1 | 1.183165 |
| 416 | 3824 | MAPRE3 | 1.183387 |
| 417 | 3624 | CTNNB1 | 1.18419 |
| 418 | 3151 | MLH1 | 1.185254 |
| 419 | 3495 | FGFR2 | 1.185537 |
| 420 | 3349 | IMPDH1 | 1.185827 |
| 421 | 2932 | MAPKAPK3 | 1.186058 |
| 422 | 3130 | FRAP1 | 1.186158 |
| 423 | 3714 | | 1.188036 |
| 424 | 3467 | MAP2K7 | 1.188179 |
| 425 | 3727 | GPRK6 | 1.188457 |
| 426 | 3500 | SGK | 1.189014 |
| 427 | 3638 | STAT5A | 1.189492 |
| 428 | 3242 | PRKCB1 | 1.191673 |
| 429 | 3588 | RHEB | 1.194214 |
| 430 | 2940 | DCAMKL1 | 1.194443 |
| 431 | 3222 | PTTG1 | 1.194583 |
| 432 | 3411 | HIF1A | 1.194933 |
| 433 | 2952 | PRKG1 | 1.197336 |
| 434 | 3539 | PLCG2 | 1.198326 |
| 435 | 3797 | ARHGEF9 | 1.20036 |
| 436 | 2969 | | 1.201116 |
| 437 | 3194 | RARB | 1.201145 |
| 438 | 3490 | ERBB3 | 1.202371 |
| 439 | 3197 | ARHB | 1.2033 |
| 440 | 3347 | TOP1 | 1.203483 |
| 441 | 2966 | | 1.203678 |
| 442 | 3089 | KIFC1 | 1.204418 |
| 443 | 3232 | KDR | 1.205127 |
| 444 | 3090 | KIF3C | 1.205488 |
| 445 | 3599 | DTYMK | 1.205645 |
| 446 | 3139 | | 1.206674 |
| 447 | 3695 | PRKAA1 | 1.20878 |
| 448 | 3425 | DLG7 | 1.209098 |
| 449 | 3535 | SKP2 | 1.20949 |
| 450 | 3327 | CDC45L | 1.209854 |
| 451 | 3651 | VRK1 | 1.210029 |
| 452 | 3569 | RASGRP2 | 1.210373 |
| 453 | 3246 | RPS6KB1 | 1.210471 |
| 454 | 3131 | KIF1B | 1.21146 |
| 455 | 3671 | STK4 | 1.212033 |
| 456 | 3757 | CLK4 | 1.212447 |
| 457 | 2985 | MKNK1 | 1.212627 |
| 458 | 2988 | FRK | 1.213049 |
| 459 | 3432 | PRC1 | 1.2136 |
| 460 | 3699 | | 1.214212 |
| 461 | 3008 | SGK2 | 1.21451 |
| 462 | 2996 | MAPK3 | 1.217258 |
| 463 | 3399 | HSPCB | 1.217278 |
| 464 | 3610 | LEF1 | 1.219128 |
| 465 | 2980 | RIPK1 | 1.220712 |
| 466 | 3675 | ADRBK1 | 1.22227 |
| 467 | 3663 | ALS2CR2 | 1.223782 |
| 468 | 3468 | ABL2 | 1.223785 |
| 469 | 2961 | PIM1 | 1.223874 |
| 470 | 3804 | | 1.224331 |
| 471 | 3594 | RAP2A | 1.226644 |
| 472 | 3377 | | 1.227759 |
| 473 | 3341 | APLP2 | 1.229895 |
| 474 | 3524 | NOTCH3 | 1.230078 |
| 475 | 3253 | PRKCA | 1.233866 |
| 476 | 3518 | NFKB1 | 1.23456 |
| 477 | 3328 | CCNC | 1.236473 |
| 478 | 3563 | RAB3A | 1.237081 |
| 479 | 3765 | CREBBP | 1.23722 |
| 480 | 2979 | PAK2 | 1.237809 |
| 481 | 3235 | CHEK1 | 1.239472 |
| 482 | 3146 | KIF21A | 1.239694 |
| 483 | 3340 | CENPH | 1.239979 |
| 484 | 3215 | MAD2L1 | 1.24524 |
| 485 | 3379 | | 1.245359 |
| 486 | 3662 | LCK | 1.245594 |
| 487 | 3754 | CDKL2 | 1.247567 |
| 488 | 3187 | WNT7B | 1.247786 |
| 489 | 3552 | PDK1 | 1.248615 |
| 490 | 3618 | DVL2 | 1.249105 |
| 491 | 3602 | MCM3 | 1.249136 |
| 492 | 3564 | RALBP1 | 1.249919 |
| 493 | 3404 | PPARG | 1.252208 |
| 494 | 3248 | JAK2 | 1.252557 |
| 495 | 3147 | CDKN2A | 1.252718 |
| 496 | 3358 | TOP2B | 1.253573 |
| 497 | 3459 | EGFR | 1.255853 |
| 498 | 3249 | MAP2K6 | 1.256087 |
| 499 | 3254 | CSF1R | 1.258457 |
| 500 | 2949 | MYO3B | 1.259934 |
| 501 | 3157 | NF1 | 1.260606 |
| 502 | 3680 | CLK3 | 1.262403 |
| 503 | 3113 | CNK | 1.262742 |
| 504 | 3825 | | 1.263089 |
| 505 | 3667 | | 1.26407 |
| 506 | 3753 | CDK5 | 1.264077 |
| 507 | 3553 | CKS1B | 1.265301 |
| 508 | 2933 | MAP4K5 | 1.265655 |
| 509 | 3796 | ARHGEF6 | 1.265751 |
| 510 | 3419 | RFC4 | 1.266922 |
| 511 | 3460 | CSK | 1.266969 |
| 512 | 3094 | KIF3A | 1.26728 |
| 513 | 3736 | PTK7 | 1.267303 |
| 514 | 3707 | TXK | 1.268516 |
| 515 | 3791 | | 1.26868 |
| 516 | 3523 | NOTCH2 | 1.26965 |
| 517 | 3755 | CDKL3 | 1.271644 |
| 518 | 3204 | CDC16 | 1.271646 |
| 519 | 3353 | PGR | 1.271733 |
| 520 | 3115 | MDM2 | 1.274517 |
| 521 | 3126 | KIF3B | 1.274522 |
| 522 | 3095 | KIF2C | 1.274859 |
| 523 | 2947 | | 1.277515 |
| 524 | 3408 | PIN1 | 1.278984 |
| 525 | 3657 | PCTK1 | 1.279578 |
| 526 | 3211 | BUB1B | 1.282741 |
| 527 | 3643 | DDR2 | 1.28316 |
| 528 | 3449 | TBK1 | 1.285291 |
| 529 | 3669 | NTRK3 | 1.285519 |
| 530 | 3200 | REL | 1.285524 |
| 531 | 3729 | RYK | 1.291213 |
| 532 | 3691 | | 1.291243 |
| 533 | 3214 | CENPF | 1.291507 |
| 534 | 3801 | PSEN1 | 1.291634 |
| 535 | 2978 | | 1.293122 |
| 536 | 3141 | | 1.294102 |
| 537 | 3792 | ARHGEF1 | 1.294579 |
| 538 | 3477 | FGFR4 | 1.29633 |
| 539 | 3169 | NME1 | 1.298008 |
| 540 | 3693 | NEK9 | 1.299989 |
| 541 | 3583 | JUNB | 1.300395 |
| 542 | 3768 | ARAF1 | 1.302137 |
| 543 | 2975 | NEK4 | 1.302558 |
| 544 | 3221 | TOP3B | 1.30285 |
| 545 | 3478 | EPHA2 | 1.30329 |
| 546 | 3666 | PAK6 | 1.303653 |
| 547 | 2963 | MAP3K11 | 1.306508 |
| 548 | 3199 | NF2 | 1.307337 |
| 549 | 3724 | EPHA7 | 1.308035 |
| 550 | 3457 | BAD | 1.308937 |
| 551 | 3185 | VCAM1 | 1.309542 |
| 552 | 3244 | PRKCZ | 1.310965 |
| 553 | 3587 | VAV3 | 1.31294 |
| 554 | 3712 | RPS6KA6 | 1.313627 |
| 555 | 3216 | CDC20 | 1.315701 |
| 556 | 3551 | STAT3 | 1.316414 |
| 557 | 3590 | ARHGEF2 | 1.316547 |
| 558 | 3659 | | 1.317441 |
| 559 | 3831 | CLSPN | 1.318145 |
| 560 | 3109 | KIF1C | 1.318847 |
| 561 | 3455 | MAP2K4 | 1.319428 |
| 562 | 3118 | | 1.319892 |
| 563 | 3709 | | 1.320996 |
| 564 | 3122 | ATSV | 1.321439 |
| 565 | 3809 | CDCA3 | 1.329173 |
| 566 | 3237 | CDC7 | 1.330145 |
| 567 | 3650 | | 1.335065 |
| 568 | 3382 | TUBB | 1.336093 |
| 569 | 3190 | WNT4 | 1.336703 |
| 570 | 3591 | RALB | 1.338466 |
| 571 | 3091 | KIFC3 | 1.339318 |
| 572 | 3761 | WT1 | 1.340453 |
| 573 | 3832 | ATM | 1.343275 |
| 574 | 3154 | MADH4 | 1.343448 |
| 575 | 3002 | CRKL | 1.345404 |
| 576 | 2946 | | 1.346714 |
| 577 | 3389 | | 1.347114 |
| 578 | 3645 | CASK | 1.34749 |
| 579 | 3315 | CCT4 | 1.348613 |
| 580 | 3150 | BRCA2 | 1.34964 |
| 581 | 3474 | CSNK2A1 | 1.350654 |
| 582 | 3458 | ARAF1 | 1.351534 |
| 583 | 3528 | TCF3 | 1.354214 |
| 584 | 3529 | DTX1 | 1.357117 |
| 585 | 3559 | RAP1GDS1 | 1.359432 |
| 586 | 3721 | ANKRD3 | 1.361311 |
| 587 | 3819 | TACC3 | 1.367136 |
| 588 | 3578 | RREB1 | 1.367845 |
| 589 | 3245 | AXL | 1.367989 |
| 590 | 3543 | PDK2 | 1.368231 |
| 591 | 3352 | NR3C2 | 1.368397 |
| 592 | 3493 | MAPK9 | 1.368899 |
| 593 | 2958 | PRKACA | 1.371294 |
| 594 | 3435 | FLT3 | 1.371521 |
| 595 | 3316 | CCNA1 | 1.37217 |
| 596 | 3263 | CDC2 | 1.373177 |
| 597 | 3224 | FBXO5 | 1.374389 |
| 598 | 2701 | | 1.378002 |
| 599 | 3497 | EPHA8 | 1.378119 |
| 600 | 3255 | CDK7 | 1.379045 |
| 601 | 3766 | S100A2 | 1.379101 |
| 602 | 3690 | PRKAA2 | 1.383537 |
| 603 | 3152 | APC | 1.384859 |
| 604 | 3201 | RARA | 1.387549 |
| 605 | 3396 | HDAC10 | 1.391302 |
| 606 | 3363 | GART | 1.392568 |
| 607 | 2957 | TYK2 | 1.392639 |
| 608 | 3323 | CDK5R1 | 1.394769 |
| 609 | 3380 | TUBG2 | 1.401159 |
| 610 | 3233 | ROCK1 | 1.404806 |
| 611 | 3806 | C10orf3 | 1.405976 |
| 612 | 3614 | CTNND2 | 1.409777 |
| 613 | 3093 | PIK3CG | 1.41077 |
| 614 | 3763 | | 1.412316 |
| 615 | 3487 | MAP2K3 | 1.412348 |
| 616 | 3732 | CSNK2A2 | 1.414035 |
| 617 | 3110 | KIF13A | 1.414042 |
| 618 | 3789 | ELK1 | 1.414448 |
| 619 | 3786 | FRAP1 | 1.416676 |
| 620 | 3554 | PIK3R3 | 1.418107 |
| 621 | 3167 | S100A2 | 1.418532 |
| 622 | 3084 | KIF14 | 1.41956 |
| 623 | 3661 | FGR | 1.423887 |
| 624 | 3617 | CTNNBIP1 | 1.425161 |
| 625 | 2974 | NEK11 | 1.426945 |
| 626 | 3330 | CDK9 | 1.428872 |
| 627 | 3227 | NUMA1 | 1.432118 |
| 628 | 3734 | BMPR1B | 1.437299 |
| 629 | 3138 | KIF17 | 1.441566 |
| 630 | 3186 | ETS1 | 1.442612 |
| 631 | 3673 | DDR1 | 1.444582 |
| 632 | 3450 | MAP3K2 | 1.446117 |
| 633 | 3133 | | 1.450561 |
| 634 | 3598 | PCNA | 1.451899 |
| 635 | 3106 | KIF9 | 1.45407 |
| 636 | 3608 | MAP3K7IP1 | 1.455728 |
| 637 | 3376 | AGA | 1.457466 |
| 638 | 3443 | EPS8 | 1.460769 |
| 639 | 3102 | CKS2 | 1.464872 |
| 640 | 3409 | TTK | 1.465445 |
| 641 | 3346 | CCNK | 1.465948 |
| 642 | 3604 | TK2 | 1.466617 |
| 643 | 2921 | RPS6KA5 | 1.467418 |
| 644 | 3597 | SHMT2 | 1.468236 |
| 645 | 3499 | GRB2 | 1.469395 |
| 646 | 3406 | TERT | 1.482496 |
| 647 | 3158 | BRCA1 | 1.485158 |
| 648 | 3114 | PIK3CD | 1.485825 |
| 649 | 3575 | LATS2 | 1.487058 |
| 650 | 3390 | | 1.487135 |
| 651 | 3596 | SHMT1 | 1.487573 |
| 652 | 3514 | HRAS | 1.488051 |
| 653 | 3730 | TESK1 | 1.489848 |
| 654 | 3620 | AXIN2 | 1.491167 |
| 655 | 3619 | FRAT1 | 1.491691 |
| 656 | 3644 | EPHB1 | 1.492026 |
| 657 | 3117 | ATM | 1.498897 |
| 658 | 3541 | PKD2 | 1.5005 |
| 659 | 3607 | TLE1 | 1.501123 |
| 660 | 3229 | PRKCL1 | 1.502059 |
| 661 | 3104 | CDK4 | 1.502301 |
| 662 | 3684 | STK38L | 1.5024 |
| 663 | 3626 | DVL3 | 1.504253 |
| 664 | 2986 | ACVR2 | 1.510627 |
| 665 | 2971 | DAPK2 | 1.516585 |
| 666 | 3759 | | 1.517994 |
| 667 | 3636 | STAT2 | 1.519082 |
| 668 | 3611 | CTNNAL1 | 1.523973 |
| 669 | 3794 | WASL | 1.529001 |
| 670 | 2944 | MARK1 | 1.53037 |
| 671 | 3713 | PRKG2 | 1.535337 |
| 672 | 3087 | PTEN | 1.540121 |
| 673 | 3506 | | 1.54045 |
| 674 | 3191 | WNT2 | 1.553178 |
| 675 | 3202 | MCC | 1.554866 |
| 676 | 3210 | | 1.555868 |
| 677 | 3738 | PRKWNK3 | 1.559877 |
| 678 | 3096 | AKT1 | 1.560781 |
| 679 | 3308 | CDKN2B | 1.566367 |
| 680 | 3606 | CREBBP | 1.570055 |
| 681 | 3641 | TYRO3 | 1.574144 |
| 682 | 3758 | RAD51L1 | 1.575115 |
| 683 | 3192 | WNT1 | 1.579448 |
| 684 | 3705 | | 1.591723 |
| 685 | 3565 | RAB2 | 1.597556 |
| 686 | 3770 | TGFBR2 | 1.602887 |
| 687 | 3771 | PIK3CA | 1.605624 |
| 688 | 3314 | CCNG1 | 1.606354 |
| 689 | 3579 | PDZGEF2 | 1.609017 |
| 690 | 3603 | POLS | 1.609696 |
| 691 | 3589 | SOS1 | 1.610658 |
| 692 | 2938 | ALS2CR7 | 1.613751 |
| 693 | 3621 | CTNNA2 | 1.62379 |
| 694 | 3265 | RAF1 | 1.625904 |
| 695 | 3698 | ADRBK2 | 1.626836 |
| 696 | 3622 | FZD9 | 1.630823 |
| 697 | 3111 | KIF5B | 1.633474 |
| 698 | 3688 | GUCY2D | 1.63373 |
| 699 | 3489 | SRC | 1.633931 |
| 700 | 3320 | CCND3 | 1.636023 |
| 701 | 2970 | AATK | 1.636349 |
| 702 | 3562 | RASD1 | 1.636677 |
| 703 | 3728 | TIE | 1.637314 |
| 704 | 3827 | | 1.638302 |
| 705 | 3778 | VHL | 1.639913 |
| 706 | 3448 | | 1.6515 |
| 707 | 3426 | TK1 | 1.654609 |
| 708 | 2701 | | 1.655539 |
| 709 | 3331 | CDC25B | 1.661891 |
| 710 | 3371 | | 1.664564 |
| 711 | 2923 | ERN1 | 1.665407 |
| 712 | 3550 | PLCG1 | 1.667241 |
| 713 | 3803 | MPHOSPH1 | 1.668632 |
| 714 | 3333 | CCNT2 | 1.669848 |
| 715 | 3520 | NRAS | 1.670763 |
| 716 | 3121 | PIK3C2A | 1.675061 |
| 717 | 3264 | CDK3 | 1.681459 |
| 718 | 3785 | GRB2 | 1.681539 |
| 719 | 3205 | | 1.682938 |
| 720 | 3811 | | 1.685119 |
| 721 | 3507 | | 1.688535 |
| 722 | 2955 | PAK1 | 1.688691 |
| 723 | 3440 | FGFR3 | 1.695183 |
| 724 | 2994 | MATK | 1.696094 |
| 725 | 2967 | | 1.703715 |
| 726 | 3325 | CDKN1C | 1.703926 |
| 727 | 3545 | IRS2 | 1.705996 |
| 728 | 3492 | PRKCQ | 1.706638 |
| 729 | 3547 | FOXO1A | 1.710389 |
| 730 | 3530 | NOTCH1 | 1.711344 |
| 731 | 3810 | | 1.723959 |
| 732 | 3321 | CDKN3 | 1.724044 |
| 733 | 3453 | MAP2K2 | 1.737418 |
| 734 | 3793 | MAPRE1 | 1.738248 |
| 735 | 2941 | DYRK3 | 1.741034 |
| 736 | 3217 | | 1.745349 |
| 737 | 3451 | MAP3K4 | 1.753145 |
| 738 | 3442 | ERBB4 | 1.760041 |
| 739 | 3696 | | 1.760172 |
| 740 | 3701 | STK10 | 1.767448 |
| 741 | 3817 | | 1.768117 |
| 742 | 2912 | MPHOSPH1 | 1.771582 |
| 743 | 3001 | PRKY | 1.772697 |
| 744 | 3128 | AKT2 | 1.773864 |
| 745 | 2981 | ALK | 1.781796 |
| 746 | 3337 | CDKN1A | 1.781903 |
| 747 | 3802 | NOTCH3 | 1.787122 |
| 748 | 3735 | PRKACB | 1.790032 |
| 749 | 3183 | | 1.793085 |
| 750 | 3430 | STMN1 | 1.798292 |
| 751 | 3531 | | 1.800094 |
| 752 | 3515 | PDGFRB | 1.80459 |
| 753 | 3324 | CUL5 | 1.820969 |
| 754 | 3511 | | 1.832738 |
| 755 | 3472 | MAP3K5 | 1.834487 |
| 756 | 3428 | ECT2 | 1.84097 |
| 757 | 3642 | EPHB3 | 1.84828 |
| 758 | 3208 | ZW10 | 1.858453 |
| 759 | 3820 | | 1.861643 |
| 760 | 3225 | | 1.868827 |
| 761 | 3834 | CHEK1 | 1.869085 |
| 762 | 3510 | CDK4 | 1.869212 |
| 763 | 3795 | NR4A2 | 1.870845 |
| 764 | 3247 | | 1.875796 |
| 765 | 3521 | MET | 1.887521 |
| 766 | 3538 | NFKB2 | 1.892227 |
| 767 | 3818 | | 1.900799 |
| 768 | 3719 | BMPR2 | 1.919267 |
| 769 | 3144 | KIF4B | 1.924148 |
| 770 | 3355 | GUK1 | 1.925235 |
| 771 | 2956 | PRKCL2 | 1.929173 |
| 772 | 3198 | ICAM1 | 1.937953 |
| 773 | 3361 | IMPDH2 | 1.938577 |
| 774 | 3672 | SYK | 1.945812 |
| 775 | 3697 | CAMK2G | 1.946161 |
| 776 | 3415 | HSPCA | 1.94686 |
| 777 | 3505 | STK6 | 1.949702 |
| 778 | 3368 | TOP3A | 1.959095 |
| 779 | 3681 | SRPK1 | 1.963919 |
| 780 | 3613 | DVL1 | 1.984151 |
| 781 | 3720 | | 1.996699 |
| 782 | 2995 | PTK2 | 2.015836 |
| 783 | 3522 | KRAS2 | 2.026984 |
| 784 | 3436 | BRAF | 2.036457 |
| 785 | 3787 | FZD4 | 2.049799 |
| 786 | 3584 | RASAL2 | 2.089191 |
| 787 | 3098 | CENPE | 2.090255 |
| 788 | 3267 | CCNH | 2.096356 |
| 789 | 2931 | MAP4K3 | 2.11675 |
| 790 | 2962 | MAP4K2 | 2.12521 |
| 791 | 3790 | ERBB3 | 2.13688 |
| 792 | 3742 | RHOK | 2.142917 |
| 793 | 2948 | MYO3A | 2.173575 |
| 794 | 3629 | AXIN1 | 2.184253 |
| 795 | 3546 | INPP5D | 2.197591 |
| 796 | 3723 | | 2.212338 |
| 797 | 2973 | NEK1 | 2.222767 |
| 798 | 3512 | TGFBR1 | 2.223853 |
| 799 | 3135 | | 2.223901 |
| 800 | 3637 | STAT4 | 2.227212 |
| 801 | 3004 | MAP3K1 | 2.235803 |
| 802 | 3304 | CCNE2 | 2.239326 |
| 803 | 3129 | STK6 | 2.248154 |
| 804 | 3402 | HDAC4 | 2.253527 |
| 805 | 3627 | CTNNA1 | 2.28197 |
| 806 | 3537 | EIF4EBP1 | 2.322458 |
| 807 | 3704 | ACVR2B | 2.322634 |
| 808 | 3329 | CDC42 | 2.333632 |
| 809 | 3259 | MAPK8 | 2.334959 |
| 810 | 3689 | BLK | 2.340679 |
| 811 | 3241 | WEE1 | 2.35419 |
| 812 | 3137 | KIF26A | 2.359341 |
| 813 | 3612 | TCF1 | 2.413867 |
| 814 | 3532 | | 2.468626 |
| 815 | 3764 | NOTCH4 | 2.482525 |
| 816 | 3417 | HDAC3 | 2.485246 |
| 817 | 3120 | PIK3CB | 2.528659 |
| 818 | 3313 | CCNG2 | 2.568855 |
| 819 | 3722 | TLK2 | 2.571781 |
| 820 | 3136 | | 2.916125 |
| 821 | 3780 | MCM3 | 2.988111 |
| 822 | 3580 | ELK1 | 3.0307 |
| 823 | 3718 | PTK6 | 3.090027 |
| 824 | 3777 | ABL1 | 3.099871 |
| 825 | 3605 | FZD4 | 3.155698 |
| 826 | 3134 | | 3.263194 |
| 827 | 2929 | CHUK | 3.298485 |
| 828 | 3781 | SRC | 3.433423 |
| 829 | 3223 | | 3.587036 |
| 830 | 3706 | C20orf97 | 4.288466 |

TABLE IIIA

siRNA sequences used in screens of DNA damaging
agents: cisplatin screen

| | | | |
|---|---|---|---|
| | SEQUENCE | | |
| GENE NAME | ID | SENSE SEQ | SEQ ID NO |

| | | | |
|---|---|---|---|
| CHUK | NM_001278 | AAAGGCUGCUCACAAGUUCTT | 50 |
| CHUK | NM_001278 | AGCUGCUCAACAAACCAGATT | 51 |
| CHUK | NM_001278 | AUGAGGAACAGGGCAAUAGTT | 52 |
| PRKACA | NM_002730 | GAAUGGGGUCAACGAUAUCTT | 53 |
| PRKACA | NM_002730 | GGACGAGACUUCCUCUUGATT | 54 |
| PRKACA | NM_002730 | GUGUGGCAAGGAGUUUUCUTT | 55 |
| MAP4K2 | NM_004579 | GAAUCCUAAGAAGAGGCCGTT | 56 |
| MAP4K2 | NM_004579 | GAGGAGGUCUUUCAUUGGGTT | 57 |
| MAP4K2 | NM_004579 | GAUAGUCAAGCUAGACCCATT | 58 |
| STK17B | NM_004226 | AUCCUCCUGUAAUGGAACCTT | 59 |
| STK17B | NM_004226 | GAAGAGGACAGGAUUGUCGTT | 60 |
| STK17B | NM_004226 | GACCAACAGCAGAGAUAUGTT | 61 |
| ALK | NM_004304 | ACCAGAGACCAAAUGUCACTT | 62 |
| ALK | NM_004304 | AUAAGCCCACCAGCUUGUGTT | 63 |
| ALK | NM_004304 | UCAACACCGCUUUGCCGAUTT | 64 |
| FRK | NM_002031 | ACUAUAGACUUCCGCAACCTT | 65 |
| FRK | NM_002031 | CAGUAGAUUGCUGUGGCCUTT | 66 |
| FRK | NM_002031 | CUCCAUACAGCUUCUGAAGTT | 67 |
| MAP3K1 | AF042838 | UCACUUAGCAGCUGAGUCUTT | 68 |
| MAP3K1 | AF042838 | UUGACAGCACUGGUCAGAGTT | 69 |
| MAP3K1 | AF042838 | UUGGCAAGAACUUCUUGGCTT | 70 |
| KIF2C | NM_006845 | ACAAAAACGGAGAUCCGUCTT | 71 |
| KIF2C | NM_006845 | AUAAGCAGCAAGAAACGGCTT | 72 |
| KIF2C | NM_006845 | GAAUUUCGGGCUACUUUGGTT | 73 |
| CENPE | NM_001813 | GAAAAUGAAGCUUUGCGGGTT | 74 |
| CENPE | NM_001813 | GAAGAGAUCCCAGUGCUUCTT | 75 |
| CENPE | NM_001813 | UCUGAAAGUGACCAGCUCATT | 76 |
| STK6 | NM 003600 | ACAGUCUUAGGAAUCGUGCTT | 3 |
| STK6 | NM_003600 | GCACAAAAGCUUGUCUCCATT | 1 |
| STK6 | NM_003600 | UUGCAGAUUUUGGGUGGUCTT | 2 |
| KIF4B | AF241316 | CCUGCAGCAACUGAUUACCTT | 77 |
| KIF4B | AF241316 | GAACUUGAGAAGAUGCGAGTT | 78 |
| KIF4B | AF241316 | GAAGAGGCCCACUGAAGUUTT | 79 |
| BRCA2 | NM_000059 | CAAAUGGGCAGGACUCUUATT | 80 |
| BRCA2 | NM_000059 | CUGUUCAGCCCAGUUUGAATT | 81 |
| BRCA2 | NM_000059 | UCUCCAAGGAAGUUGUACCTT | 82 |
| APC | NM_000038 | ACCAAGUAUCCGCAAAAGGTT | 83 |
| APC | NM_000038 | AGACCUGUAUUAGUACGCCTT | 84 |
| APC | NM_000038 | CAAGCUUUACCCAGCCUGUTT | 85 |
| ATR | NM_001184 | GAAACUGCAGCUAUCUUCCTT | 86 |
| ATR | NM_001184 | GUUACAAUGAGGCUGAUGCTT | 87 |
| ATR | NM_001184 | UCACGACUCGCUGAACUGUTT | 88 |
| BRCA1 | NM_007296 | ACUUAGGUGAAGCAGCAUCTT | 89 |
| BRCA1 | NM_007296 | GGGCAGUGAAGACUUGAUUTT | 90 |
| BRCA1 | NM_007296 | UGAAGUGGGCUCCAGUAUUTT | 91 |
| DCC | NM_005215 | ACAUCGUGGUGCGAGGUUATT | 92 |
| DCC | NM_005215 | AUGAGCCGCCAAUUGGACATT | 93 |
| DCC | NM_005215 | AUGGCAAGUUUGGAAGGACTT | 94 |
| WNT1 | NM_005430 | ACGGCGUUUAUCUUCGCUATT | 95 |
| WNT1 | NM_005430 | CCCUCUUGCCAUCCUGAUGTT | 96 |
| WNT1 | NM_005430 | CUAUUUAUUGUGCUGGGUCTT | 97 |
| CHEK1 | NM_001274 | AUCGAUUCUGCUCCUCUAGTT | 98 |
| CHEK1 | NM_001274 | CUGAAGAAGCAGUCGCAGUTT | 99 |
| CHEK1 | NM_001274 | UGCCUGAAAGAGACUUGUGTT | 100 |
| WEE1 | NM_003390 | AUCGGCUCUGGAGAAUUUGTT | 101 |
| WEE1 | NM_003390 | CAAGGAUCUCCAGUCCACATT | 102 |
| WEE1 | NM_003390 | UGUACCUGUGUGUCCAUCUTT | 103 |
| | NM_018492 | AGGACACUUUGGGUACCAGTT | 104 |
| | NM_018492 | GACCCUAAAGAUCGUCCUUTT | 105 |
| | NM_018492 | GCUGAGGAGAAUAUGCCUCTT | 106 |
| MAPK8 | NM_139049 | CACCCGUACAUCAAUGUCUTT | 107 |
| MAPK8 | NM_139049 | GGAAUAGUAUGCGCAGCUUTT | 108 |
| MAPK8 | NM_139049 | GUGAUUCAGAUGGAGCUAGTT | 109 |
| CUL1 | NM_003592 | GACCGCAAACUACUGAUUCTT | 110 |
| CUL1 | NM_003592 | GCCAGCAUGAUCUCCAAGUTT | 111 |
| CUL1 | NM_003592 | UAGACAUUGGGUUCGCCGUTT | 112 |
| CCNG2 | NM_004354 | CCUCGAGAAAAAGGGCUGATT | 113 |
| CCNG2 | NM_004354 | GCUCAGCUGAAAGCUUGCATT | 114 |
| CCNG2 | NM_004354 | UGCCUAGCCGAGUAUUCUUTT | 115 |
| CDC42 | NM_044472 | ACCUUAUGGAAAAGGGGUGTT | 116 |
| CDC42 | NM_044472 | CCAUCCUGUUUGAAAGCCUTT | 117 |
| CDC42 | NM_044472 | CCCAAAAGGAAGUGCUGUATT | 118 |
| CDC25B | NM_021874 | AGGAUGAUGAUGCAGUUCCTT | 119 |
| CDC25B | NM_021874 | GACAAGGAGAAUGUGCGCUTT | 120 |
| CDC25B | NM_021874 | GAGCCCAGUCUGUUGAGUUTT | 121 |
| TOP2B | NM_001068 | ACAUUCCCUGGAGUGUACATT | 122 |
| TOP2B | NM_001068 | GAGGAUUUAGCGGCAUUUGTT | 123 |
| TOP2B | NM_001068 | GCUGCUGGACUGCAUAAAGTT | 124 |
| IMPDH2 | NM_000884 | AGAGGGAAGACUUGGUGGUTT | 125 |
| IMPDH2 | NM_000884 | CACUCAUGCCAGGACAUUGTT | 126 |
| IMPDH2 | NM_000884 | GAAGAAUCGGGACUACCCATT | 127 |
| | NM_007027 | ACUCACAGAAAAACCGUCGTT | 128 |
| | NM_007027 | AUGAUGGGCGGACGAGUAUTT | 129 |
| | NM_007027 | GAGUCAGCACCAUCAAAUGTT | 130 |
| HDAC4 | NM_006037 | AGAGGACGUUUUCUACGGCTT | 131 |
| HDAC4 | NM_006037 | AUCUGUUUGCAAGGGGAAGTT | 132 |
| HDAC4 | NM_006037 | CAAGAUCAUCCCCAAGCCATT | 133 |
| TERT | NM_003219 | CACCAAGAAGUUCAUCUCCTT | 134 |
| TERT | NM_003219 | GAGUGUCUGGAGCAAGUUGTT | 135 |
| TERT | NM_003219 | GUUUGGAAGAACCCCACAUTT | 136 |
| BRAF | NM_004333 | ACACUUGGUAGACGGGACUTT | 137 |
| BRAF | NM_004333 | GUCAAUCAUCCACAGAGACTT | 138 |
| BRAF | NM_004333 | UUGCAUGUGGAAGUGUUGGTT | 139 |
| ERBB4 | NM_005235 | GAGUACUCUAUAGUGGCCUTT | 140 |
| ERBB4 | NM_005235 | GCUUCCCAGUCCAAAUGACTT | 141 |
| ERBB4 | NM_005235 | UGACAGUGGAGCAUGUGUUTT | 142 |
| ABL2 | NM_007314 | AUCAGUGAUGUGGUGCAGATT | 143 |
| ABL2 | NM_007314 | GAGUCGGACACUGAAGAAATT | 144 |
| ABL2 | NM_007314 | UGGCACAGCAGGUACUAAATT | 145 |
| KRAS2 | NM_033360 | GAAAAGACUCCUGGCUGUGTT | 146 |
| KRAS2 | NM_033360 | GGACUCUGAAGAUGUACCUTT | 147 |
| KRAS2 | NM_033360 | GGCAUACUAGUACAAGUGGTT | 148 |
| | NM_021170 | AUCCUGGAGAUGACCGUGATT | 149 |
| | NM_021170 | GCCGGUCAUGGAGAAGCGGTT | 150 |
| | NM_021170 | UGGCCCUGAGACUGCAUCGTT | 151 |
| ELK1 | NM_005229 | GCCAUUCCUUUGUCUGCCATT | 152 |
| ELK1 | NM_005229 | GUGAAAGUAGAAGGGCCCATT | 153 |
| ELK1 | NM_005229 | UUCAAGCUGGUGGAUGCAGTT | 154 |
| RASAL2 | NM_004841 | AGUACCAGGAUUCUUCAGCTT | 155 |
| RASAL2 | NM_004841 | CUUAGUUCUGGGCCAUGUATT | 156 |
| RASAL2 | NM_004841 | GACCCCACUGACAGUGAUU1T | 157 |
| ARHGEF2 | NM_004723 | AGCUACACCACAGAUGCCATT | 158 |
| ARHGEF2 | NM_004723 | GGACUUUGCAGCUGACUCUTT | 159 |
| ARHGEF2 | NM_004723 | UAAAGGUUGGGGUGGCCAUTT | 160 |
| FRAT1 | NM_005479 | AAGCUAAUGACGAGGAACCTT | 161 |
| FRAT1 | NM_005479 | CCAUGGUGAAGUGCUUGGATT | 162 |
| FRAT1 | NM_005479 | UAACAGCUGCAAUUCCCUGTT | 163 |
| CTNNA2 | NM_004389 | CCUGAUGAAUGCUGUUGUCTT | 164 |
| CTNNA2 | NM_004389 | GCACAAUACGGUGACCAAUTT | 165 |
| CTNNA2 | NM_004389 | UCACAUCUUGGAGGAUGUGTT | 166 |
| AXIN1 | AF009674 | GAAAGUGAGCGACGAGUUUTT | 167 |
| AXIN1 | AF009674 | GUGCCUUCAACACAGCUUGTT | 168 |
| AXIN1 | AF009674 | UGAAUAUCCAAGAGCAGGGTT | 169 |
| EPHB3 | NM_004443 | GAAGAUCCUGAGCAGUAUCTT | 170 |
| EPHB3 | NM_004443 | GCUGCAGCAGUACAUUGCUTT | 171 |
| EPHB3 | NM_004443 | UACCCUGGACAAGCUCAUCTT | 172 |
| DDR1 | NM_013994 | AACAAGAGGACACAAUGGCTT | 173 |
| DDR1 | NM_013994 | AGAGGUGAAGAUCAUGUCGTT | 174 |
| DDRI | NM_013994 | UCGCAGACUUUGGCAUGAGTT | 175 |
| CLK2 | NM_003993 | AUCGUUAGCACCUUAGGAGTT | 176 |
| CLK2 | NM_003993 | CCCCUGCCUUGUACAUAAUTT | 177 |
| CLK2 | NM_003993 | GUACAAGGAAGCAGCUCGATT | 178 |
| C20orf97 | NM_021158 | AGUCCCAGGUGGGACUCUUTT | 179 |
| C20orf97 | NM_021158 | CUGGCAUCCUUGAGCUGACTT | 180 |
| C20orf97 | NM_021158 | GACUGUUCUGGAAUGAGGGTT | 181 |
| | X95425 | ACUGCCAGGAGUAAGAACUTT | 182 |
| | X95425 | CUAUUACUGCAGAGGGCUUTT | 183 |
| | X95425 | UGCAUCCUGCAGAGUAUCUTT | 184 |
| RPS6KA6 | NM_014496 | CCUCCUUUCAAACCUGCUUTT | 185 |
| RPS6KA6 | NM_014496 | GAGGUUCUGUUUACAGAGGTT | 186 |
| RPS6KA6 | NM_014496 | UCAGCCAGUGCAGAUUCAATT | 187 |
| | AB002301 | AGACAAAGAGGGGACCUUCTT | 188 |
| | AB002301 | GAAAGUCUAUCCGAAGGCUTT | 189 |
| | AB002301 | UGCCUCCCUGAAACUUCGATr | 190 |
| GPRK6 | NM_002082 | AAGCAAGAAAUGGCGGCAGTT | 191 |
| GPRK6 | NM_002082 | GAGCUGAAUGUCUUUGGGCTT | 192 |
| GPRK6 | NM_002082 | UGUAUAUAGCGACCAGAGCTT | 193 |
| GSK3A | NM_019884 | CUUCAGUGCUGGUGAACUCTT | 194 |
| GSK3A | NM_019884 | GCUGGACCACUGCAAUAUUTT | 195 |
| GSK3A | NM_019884 | GUGGCUUACACGGACAUCATT | 196 |
| RAD51L1 | NM_133510 | AACAGGACCGUACUGCUUGTT | 197 |
| RAD51L1 | NM_133510 | GAAGCCUUUGUUCAGGUCUTT | 198 |
| RAD51L1 | NM_133510 | GAGAGGCAUCCUCCUUGAATT | 199 |
| NOTCH4 | NM_004557 | CCAGCACUGACUACUGUGUTT | 200 |
| NOTCH4 | NM_004557 | GGAACUCGAUGCUUGUCAGTT | 201 |
| NOTCH4 | NM_004557 | UGCGAGGAAGAUACGGAGUTT | 202 |
| MCM3 | NM_002388 | GCAGAUGAGCAAGGAUGCUTT | 203 |
| MCM3 | NM_002388 | GUACAUCCAUGUGGCCAAATT | 204 |
| MCM3 | NM_002388 | UGGGUCAUGAAAGCUGCCATT | 205 |
| FZD4 | NM_012193 | AGAACCUCGGCUACAACGUTT | 206 |
| FZD4 | NM_012193 | UCCGCAUCUCCAUGUGCCATT | 207 |
| FZD4 | NM_012193 | UCGGCUACAACGUGACCAATT | 208 |

TABLE IIIB

siRNA sequences used in screens of DNA damaging
agents: doxorubicin screen

| | | | |
|---|---|---|---|
| GENE_ | | | SEQ ID |
| SYMBOL | SEQUENCE_ID | SENSE_SEQ | NO |

| | | | |
|---|---|---|---|
| AATK | AB014541 | CGCAAGAAGAAGGCCGUGUTT | 209 |
| AATK | AB014541 | CGCUGGUGCAAUGUUUUCUTT | 210 |
| AATK | AB014541 | GAAUCCCUACCGAGACUCUTT | 211 |
| ABL1 | NM_007313 | AAACCUCUACACGUUCUGCTT | 212 |
| ABL1 | NM_007313 | CUAAAGGUGAAAAGCUCCGTT | 213 |
| ABL1 | NM_007313 | UCCUGGCAAGAAAGCUUGATT | 214 |
| ACVR2 | NM_001616 | AAGAUGGCCACAAACCUGCTT | 215 |
| ACVR2 | NM_001616 | AGAUAAACGGCGGCAUUGUTT | 216 |
| ACVR2 | NM_001616 | GACAUGCAGGAAGUUGUUGTT | 217 |
| ACVR2B | NM_001106 | CGGGAGAUCUUCAGCACACTT | 218 |
| ACVR2B | NM_001106 | GAGAUUGGCCAGCACCCUUTT | 219 |
| ACVR2B | NM_001106 | GCCCAGGACAUGAGUGUCUTT | 220 |
| ADRBK2 | NM_005160 | CGAGGAUGAGGCAUCUGAUTT | 221 |
| ADRBK2 | NM_005160 | CUGAAGUCCCUUUUGGAGGTT | 222 |
| ADRBK2 | NM_005160 | GAACUUCCCUUUGGUCAUCTT | 223 |
| AKT1 | NM_005163 | GCUGGAGAACCUCAUGCUGTT | 224 |
| AKT1 | NM_005163 | AGACGUUUUUGUGCUGUGGTT | 225 |
| AKT1 | NM_005163 | CGCACCUUCCAUGUGGAGATT | 226 |
| AKT2 | NM_001626 | AGAUGGCCACAUCAAGAUCTT | 227 |
| AKT2 | NM_001626 | GUCAUCAUUGCCAAGGAUGTT | 228 |
| AKT2 | NM_001626 | UGCCAGCUGAUGAAGACCGTT | 229 |
| ALK | NM_004304 | ACCAGAGACCAAAUGUCACTT | 230 |
| ALK | NM_004304 | AUAAGCCCACCAGCUUGUGTT | 231 |
| ALK | NM_004304 | UCAACACCGCUUUGCCGAUTT | 232 |
| ALS2CR7 | NM_139158 | CUGGCUGAUUUUGGUCUUGTT | 233 |
| ALS2CR7 | NM_139158 | GCCUUCAUGUUGUCUGGAATT | 234 |
| ALS2CR7 | NM_139158 | UCCACACCAAAGAGACACUTT | 235 |
| AXIN1 | AF009674 | GAAAGUGAGCGACGAGUUUTT | 236 |
| AXIN1 | AF009674 | GUGCCUUCAACACAGCUUGTT | 237 |
| AXIN1 | AF009674 | UGAAUAUCCAAGAGCAGGGTT | 238 |
| BLK | NM_001715 | AGUCACGAGCGUUCGAAAATT | 239 |
| BLK | NM_001715 | CAACAUGAAGGUGGCCAUUTT | 240 |
| BLK | NM_001715 | GCACUAUAAGAUCCGCUGCTT | 241 |
| BMPR2 | NM_001204 | CAAAUCUGUGAGCCCAACATT | 242 |
| BMPR2 | NM_001204 | CAAGAUGUUCUUGCACAGGTT | 243 |
| BMPR2 | NM_001204 | GAACGGCUAUGUGCGUUUATT | 244 |
| BRAF | NM_004333 | ACACUUGGUAGACGGGACUTT | 245 |
| BRAF | NM_004333 | GUCAAUCAUCCACAGAGACTT | 246 |
| BRAF | NM_004333 | UUGCAUGUGGAAGUGUUGGTT | 247 |
| C20orf97 | NM_021158 | AGUCCCAGGUGGGACUCUUTT | 248 |
| C20orf97 | NM_021158 | CUGGCAUCCUUGAGCUGACTT | 249 |
| C20orf97 | NM_021158 | GACUGUUCUGGAAUGAGGGTT | 250 |
| CAMK2G | BC021269 | GACAUUGUGGCCAGAGAGUTT | 251 |
| CAMK2G | BC021269 | GAUGAGGACCUCAAAGUGCTT | 252 |
| CAMK2G | BC021269 | GGCUGGAGCCUAUGAUUUCTT | 253 |
| CCND3 | NM_001760 | AAAGCAUGCCCAGACCUUUTT | 254 |
| CCND3 | NM_001760 | AAGGAUCUUUGUGGCCAAGTT | 255 |
| CCND3 | NM_001760 | CUACCUGGAUCGCUACCUGTT | 256 |
| CCNE2 | NM_057749 | CCACAGAUGAGGUCCAUACTT | 257 |
| CCNE2 | NM_057749 | CUGGGGCUUUCUUGACAUGTT | 258 |
| CCNE2 | NM_057749 | GUGGUUAAGAAAGCCUCAGTT | 259 |
| CCNG1 | NM_004060 | AUGGAUUGUUUCUGGGCGUTT | 260 |
| CCNG1 | NM_004060 | CUAUCAGUCUUCCCACAGCTT | 261 |
| CCNG1 | NM_004060 | CUUGCCACUUGAAAGGAGATT | 262 |
| CCNG2 | NM_004354 | CCUCGAGAAAAAGGGCUGATT | 263 |
| CCNG2 | NM_004354 | GCUCAGCUGAAAGCUUGCATT | 264 |
| CCNG2 | NM_004354 | UGCCUAGCCGAGUAUUCUUTT | 265 |
| CCNH | NM_001239 | GACCCGCUAUCCCAUAUUGTT | 266 |
| CCNH | NM_001239 | GCCAGCAAUGCCAAGAUCUTT | 267 |
| CCNH | NM_001239 | UUGCCCUGACUGCCAUUUUTT | 268 |
| CCNT2 | NM_058241 | AGCGCCAGUAAAGAAGAACTT | 269 |
| CCNT2 | NM_058241 | AGGGCAGCCAGUUGUCAUUTT | 270 |
| CCNT2 | NM_058241 | CCACCACUCCAAAAUGAGCTT | 271 |
| CDC25B | NM_021874 | AGGAUGAUGAUGCAGUUCCTT | 272 |
| CDC25B | NM_021874 | GACAAGGAGAAUGUGCGCUTT | 273 |
| CDC25B | NM_021874 | GAGCCCAGUCUGUUGAGUUTT | 274 |
| CDC42 | NM_044472 | ACCUUAUGGAAAAGGGGUGTT | 275 |
| CDC42 | NM_044472 | CCAUCCUGUUUGAAAGCCUTT | 276 |
| CDC42 | NM_044472 | CCCAAAAGGAAGUGCUGUATT | 277 |
| CDK3 | NM_001258 | CGAGAGGAAGCUCUAUCUGTT | 278 |
| CDK3 | NM_001258 | GAGAGGAUGCAUCUGGGGATT | 279 |
| CDK3 | NM_001258 | GAUCAGACUGGAUUUGGAGTT | 280 |
| CDK4 | NM_000075 | CAGUCAAGCUGGCUGACUUTT | 281 |
| CDK4 | NM_000075 | GCGAAUCUCUGCCUUUCGATT | 282 |
| CDK4 | NM_000075 | GGAUCUGAUGCGCCAGUUUTT | 283 |
| CDK4 | NM_000075 | CCCUGGUGUUUGAGCAUGUTT | 284 |
| CDK4 | NM_000075 | CUGACCGGGAGAUCAAGGUTT | 285 |
| CDK4 | NM_000075 | GAGUGUGAGAGUCCCCAAUTT | 286 |
| CDKN1A | NM_078467 | AACUAGGCGGUUGAAUGAGTT | 287 |
| CDKN1A | NM_078467 | CAUACUGGCCUGGACUGUUTT | 288 |
| CDKN1A | NM_078467 | GAUGGUGGCAGUAGAGGCUTT | 289 |
| CDKN1C | NM_000076 | AAAAACCGGGAUUCCGGCCTT | 290 |
| CDKN1C | NM_000076 | GCGCAAGAGAUCAGCGCCUTT | 291 |
| CDKN1C | NM_000076 | GUGGACAGCGACUCGGUGCTT | 292 |
| CDKN2B | NM_004936 | ACACAGAGAAGCGGAUUUCTT | 293 |
| CDKN2B | NM_004936 | CUCCAAGAGGUGGGUAAUUTT | 294 |
| CDKN2B | NM_004936 | UGUCUGCUGAGGAGUUAUGTT | 295 |
| CDKN3 | NM_005192 | CCUGCCUUAAAAAUUACCGTT | 296 |
| CDKN3 | NM_005192 | GAACUAAAGAGCUGUGGUATT | 297 |
| CDKN3 | NM_005192 | GAGGAUCCGGGGCAAUACATT | 298 |
| CENPE | NM_001813 | GAAAAUGAAGCUUUGCGGGTT | 299 |
| CENPE | NM_001813 | GAAGAGAUCCCAGUGCUUCTT | 300 |
| CENPE | NM_001813 | UCUGAAAGUGACCAGCUCATT | 301 |
| CHEK1 | NM_001274 | CCAGUUGAUGUUUGGUCCUTT | 302 |
| CHEK1 | NM_001274 | UCUCAGACUUUGGCUUGGCTT | 303 |
| CHEK1 | NM_001274 | UUCUAUGGUCACAGGAGAGTT | 304 |
| CHUK | NM_001278 | AAAGGCUGCUCACAAGUUCTT | 305 |
| CHUK | NM_001278 | AGCUGCUCAACAAACCAGATT | 306 |
| CHUK | NM_001278 | AUGAGGAACAGGGCAAUAGTT | 307 |
| CREBBP | NM_004380 | GACAUCCCGAGUCUAUAAGTT | 308 |
| CREBBP | NM_004380 | GCACAAGGAGGUCUUCUUCTT | 309 |
| CREBBP | NM_004380 | UGGAGGAGAAUUAGGCCUUTT | 310 |
| CTNNA1 | NM_001903 | CGUUCCGAUCCUCUAUACUTT | 311 |
| CTNNA1 | NM_001903 | UGACAUCAUUGUGCUGGCCTT | 312 |
| CTNNA1 | NM_001903 | UGACCAAAGAUGACCUGUGTT | 313 |
| CTNNA2 | NM_004389 | CCUGAUGAAUGCUGUUGUCTT | 314 |
| CTNNA2 | NM_004389 | GCACAAUACGGUGACCAAUTT | 315 |
| CTNNA2 | NM_004389 | UCACAUCUUGGAGGAUGUGTT | 316 |
| CTNNAL1 | NM_003798 | AAGUGUUGUUGCUGGCAGATT | 317 |
| CTNNAL1 | NM_003798 | ACUUGAGAAGCUUUUGGGGTT | 318 |
| CTNNAL1 | NM_003798 | CUAGAGGUUUUUGCUGCAGTT | 319 |
| CUL5 | NM_003478 | AAGAGUGAGCUGGUCAAUGTT | 320 |
| CUL5 | NM_003478 | AUUUUGGAGUGCUUGGGCATT | 321 |
| CUL5 | NM_003478 | UGGGUAAACAGGGCAGCAATT | 322 |
| DAPK2 | NM_014326 | GAAUAUUUUUGGGACGCCGTT | 323 |
| DAPK2 | NM_014326 | UCCAAGAGGCUCUCAGACATT | 324 |
| DAPK2 | NM_014326 | UCUCAGAAGGUCCUCCUGATT | 325 |
| DVL1 | NM_004421 | GGAGGAGAUCUUUGAUGACTT | 326 |
| DVL1 | NM_004421 | GUACGCCAGCAGCUUGCUGTT | 327 |
| DVL1 | NM_004421 | UCGGAUCACACGGCACCGATT | 328 |
| DVL3 | NM_004423 | ACCCCAGUGAGUUCUUUGUTT | 329 |
| DVL3 | NM_004423 | CCUGGACAAUGACACAGAGTT | 330 |
| DVL3 | NM_004423 | GUUCAUUUAAGCCUCAGGGTT | 331 |
| DYRK3 | NM_003582 | CCAUGUUUGCAUGGCCUUUTT | 332 |
| DYRK3 | NM_003582 | CUUCUGGAGCAAUCCAAACTT | 333 |
| DYRK3 | NM_003582 | UCUUUGGAUGCCCUCCACATT | 334 |
| ECT2 | NM_018098 | ACUGGCUAAAGAUGCUGUGTT | 335 |
| ECT2 | NM_018098 | GACCAUGGGAAAAUUGUGGTT | 336 |
| ECT2 | NM_018098 | GCUUAGUACAGCGGGUUGATT | 337 |
| EIF4EBP1 | NM_004095 | CCACCCCUUCCUUAGGUUGTT | 338 |
| EIF4EBP1 | NM_004095 | CUCACCUGUGACCAAAACATT | 339 |
| EIF4EBP1 | NM_004095 | UAGCCCAGAAGAUAAGCGGTT | 340 |
| ELK1 | NM_005229 | GCCAUUCCUUUGUCUGCCATT | 341 |
| ELK1 | NM_005229 | GUGAAAGUAGAAGGGCCCATT | 342 |
| ELK1 | NM_005229 | UUCAAGCUGGUGGAUGCAGTT | 343 |
| EPHB3 | NM_004443 | GAAGAUCCUGAGCAGUAUCTT | 344 |
| EPHB3 | NM_004443 | GCUGCAGCAGUACAUUGCUTT | 345 |
| EPHB3 | NM_004443 | UACCCUGGACAAGCUCAUCTT | 346 |
| ERBB3 | NM_001982 | CUUUCUGAAUGGGGAGCCUTT | 347 |
| ERBB3 | NM_001982 | UACACACACCAGAGUGAUGTT | 348 |
| ERBB3 | NM_001982 | UGACAGUGGAGCCUGUGUATT | 349 |
| ERBB4 | NM_005235 | GAGUACUCUAUAGUGGCCUTT | 350 |
| ERBB4 | NM_005235 | GCUUCCCAGUCCAAAUGACTT | 351 |
| ERBB4 | NM_005235 | UGACAGUGGAGCAUGUGUUTT | 352 |
| ERN1 | NM_001433 | AAGCCUUACGGUCAUGAUGTT | 353 |
| ERN1 | NM_001433 | GAAUAAUGAAGGCCUGACGTT | 354 |
| ERN1 | NM 001433 | GAUGAUUGCGAUGGAUCCUTT | 355 |
| FGFR3 | NM_000142 | AACAUCAUCAACCUGCUGGTT | 356 |
| FGFR3 | NM_000142 | CACUUCCAGCAUUUAGCUGTT | 357 |
| FGFR3 | NM_000142 | CACUUCUUACGCAAUGCUUTT | 358 |
| FOXO1A | NM_002015 | CUAUGCGUACUGCAUAGCATT | 359 |
| FOXO1A | NM_002015 | GACAACGACACAUAGCUGGTT | 360 |
| FOXO1A | NM_002015 | UACAAGGAACCUCAGAGCCTT | 361 |
| FZD4 | NM_012193 | CCAUCUGCUUGAGCUACUUTT | 362 |
| FZD4 | NM_012193 | GUUGACUUACCUGACGGACTT | 363 |
| FZD4 | NM_012193 | UUGGCAAAGGCUCCUUGUATT | 364 |
| FZD4 | NM_012193 | AGAACCUCGGCUACAACGUTT | 365 |
| FZD4 | NM_012193 | UCCGCAUCUCCAUGUGCCATT | 366 |
| FZD4 | NM_012193 | UCGGCUACAACGUGACCAATT | 367 |
| FZD9 | NM_003508 | GACUUUCCAGACCUGGCAGTT | 368 |
| FZD9 | NM_003508 | GAUCGGGGUCUUCUCCAUCTT | 369 |
| FZD9 | NM_003508 | GGACUUCGCGCUGGUCUGGTT | 370 |
| GRB2 | NM_002086 | AUACGUCCAGGCCCUCUUUTT | 371 |
| GRB2 | NM_002086 | CGGGCAGACCGGCAUGUUUTT | 372 |
| GRB2 | NM_002086 | UGCAGCACUUCAAGGUGCUTT | 373 |
| GUCY2D | NM_000180 | GAAAUUCCCAGGGGAUCAGTT | 374 |
| GUCY2D | NM_000180 | GACAGACCGGCUGCUUACATT | 375 |
| GUCY2D | NM_000180 | GUCACGGAACUGCAUAGUGTT | 376 |
| GUK1 | NM_000858 | CGGCAAAGAUUACUACUUUTT | 377 |
| GUK1 | NM_000858 | GGAGCCCGGCCUGUUUGAUTT | 378 |
| GUK1 | NM_000858 | UCAAGAAAGCUCAAAGGACTT | 379 |
| HDAC3 | NM_003883 | CCCAGAGAUUUUUGAGGGATT | 380 |
| HDAC3 | NM_003883 | UGCCUUCAACGUAGGCGAUTT | 381 |
| HDAC3 | NM_003883 | UGGUACCUAUUAGGGAUGGTT | 382 |
| HDAC4 | NM_006037 | AGAGGACGUUUUCUACGGCTT | 383 |
| HDAC4 | NM_006037 | AUCUGUUUGCAAGGGGAAGTT | 384 |
| HDAC4 | NM_006037 | CAAGAUCAUCCCCAAGCCATT | 385 |
| HSPCA | NM_005348 | ACACCUGGAGAUAAACCCUTT | 386 |
| HSPCA | NM_005348 | CCUAUGGGUCGUGGAACAATT | 387 |
| HSPCA | NM_005348 | UAACCUUGGUACUAUCGCCTT | 388 |
| ICAM1 | NM_000201 | CAGCUAAAACCUUCCUCACTT | 389 |
| ICAM1 | NM_000201 | AACACAAAGGCCCACACUUTT | 390 |
| ICAM1 | NM_000201 | CAGAGUGGAAGACAUAUGCTT | 391 |
| IMPDH2 | NM_000884 | AGAGGGAAGACUUGGUGGUTT | 392 |
| IMPDH2 | NM_000884 | CACUCAUGCCAGGACAUUGTT | 393 |
| IMPDH2 | NM_000884 | GAAGAAUCGGGACUACCCATT | 394 |
| INPP5D | NM_005541 | AGCAUUAAGAAGCCCAGUGTT | 395 |
| INPP5D | NM_005541 | GAACAAGCACUCAGAGCAGTT | 396 |
| INPP5D | NM_005541 | UCCCAUCAACAUGGUGUCCTT | 397 |
| IRS2 | NM_003749 | CACAGCCGUUCGAUGUCCATT | 398 |
| IRS2 | NM_003749 | GUACAUCGCCAUCGACGUGTT | 399 |
| IRS2 | NM_003749 | GUACCUGAUCGCCCUCUACTT | 400 |
| KIF26A | XM_050278 | AUGCGGAAUUUGCCGUGGGTT | 401 |
| KIF26A | XM_050278 | GCACAAGCACCUGUGUGAGTT | 402 |
| KIF26A | XM_050278 | GUCGUACACCAUGAUCGGGTT | 403 |
| KIF4B | AF241316 | CCUGCAGCAACUGAUUACCTT | 404 |
| KIF4B | AF241316 | GAACUUGAGAAGAUGCGAGTT | 405 |
| KIF4B | AF241316 | GAAGAGGCCCACUGAAGUUTT | 406 |
| KIF5B | NM_004521 | AAUGCAUCUCGUGAUCGCATT | 407 |
| KIF5B | NM_004521 | AGACAGUUGGAGGAAUCUGTT | 408 |
| KIF5B | NM_004521 | AUCGGCAACUUUAGCGAGUTT | 409 |
| KRAS2 | NM_033360 | GAAAAGACUCCUGGCUGUGTT | 410 |
| KRAS2 | NM_033360 | GGACUCUGAAGAUGUACCUTT | 411 |
| KRAS2 | NM_033360 | GGCAUACUAGUACAAGUGGTT | 412 |
| MAP2K2 | NM_030662 | ACCACACCUUCAUCAAGCGTT | 413 |
| MAP2K2 | NM_030662 | AGUCAGCAUCGCGGUUCUCTT | 414 |
| MAP2K2 | NM_030662 | GAAGGAGAGCCUCACAGCATT | 415 |
| MAP3K1 | AF042838 | UCACUUAGCAGCUGAGUCUTT | 416 |
| MAP3K1 | AF042838 | UUGACAGCACUGGUCAGAGTT | 417 |
| MAP3K1 | AF042838 | UUGGCAAGAACUUCUUGGCTT | 418 |
| MAP3K4 | NM_005922 | AGAACGAUCGUCCAGUGGATT | 419 |
| MAP3K4 | NM_005922 | GGUACCUCGAUGCCAUAGUTT | 420 |
| MAP3K4 | NM_005922 | UUUUGGACUAGUGCGGAUGTT | 421 |
| MAP3K5 | NM_005923 | AGAAUUGGCAGCUGAGUUGTT | 422 |
| MAP3K5 | NM_005923 | UGCAGCAGCUAUUGCACUUTT | 423 |
| MAP3K5 | NM_005923 | UGUACAGCUUGGAAGGAUGTT | 424 |
| MAP4K2 | NM_004579 | GAAUCCUAAGAAGAGGCCGTT | 425 |
| MAP4K2 | NM_004579 | GAGGAGGUCUUUCAUUGGGTT | 7426 |
| MAP4K2 | NM_004579 | GAUAGUCAAGCUAGACCCATT | 427 |
| MAP4K3 | NM_003618 | AAUGGGAUGCUGGCAAUGATT | 428 |
| MAP4K3 | NM_003618 | AUCCUUACACGGGCCAUAATT | 429 |
| MAP4K3 | NM_003618 | CUGGACCUCUGUCAGAACUTT | 430 |
| MAPK8 | NM_139049 | CACCCGUACAUCAAUGUCUTT | 431 |
| MAPK8 | NM_139049 | GGAAUAGUAUGCGCAGCUUTT | 432 |
| MAPK8 | NM_139049 | GUGAUUCAGAUGGAGCUAGTT | 433 |
| MAPRE1 | NM_012325 | GAGUAUUAACAGCCUGGACTT | 434 |
| MAPRE1 | NM_012325 | GCUAAGCUAGAACACGAGUTT | 435 |
| MAPRE1 | NM_012325 | UAGAGGAUGUGUUUCAGCCTT | 436 |
| MARK1 | NM_018650 | ACAACAGCACUCUUCAGUCTT | 437 |
| MARK1 | NM_018650 | CUGCGAGAGCGAGUUUUACTT | 438 |
| MARK1 | NM_018650 | UGUGUAUUCUGGAGGUAGCTT | 439 |
| MATK | NM_002378 | AGCGGAAACACGGGACCAATT | 440 |
| MATK | NM_002378 | GUGUGAUGUGACAGCCCAGTT | 441 |
| MATK | NM_002378 | UGUCACUGAAAGAGGUGUCTT | 442 |
| MCC | NM_002387 | AGUUGAGGAGGUUUCUGCATT | 443 |
| MCC | NM_002387 | GACUUAGAGCUGGGAAUCUTT | 444 |
| MCC | NM_002387 | GGAUUAUAUCCAGCAGCUCTT | 445 |
| MCM3 | NM_002388 | GCAGAUGAGCAAGGAUGCUTT | 446 |
| MCM3 | NM_002388 | GUACAUCCAUGUGGCCAAATT | 447 |
| MCM3 | NM_002388 | UGGGUCAUGAAAGCUGCCATT | 448 |
| MET | NM_000245 | AUGCCUCUGGAGUGUAUUCTT | 449 |
| MET | NM_000245 | AUGCGCCCAUCCUUUUCUGTT | 450 |
| MET | NM_000245 | GAUCUGGGCAGUGAAUUAGTT | 451 |
| MPHOSPH1 | NM_016195 | AGAGGAACUCUCUGCAAGCTT | 452 |
| MPHOSPH1 | NM_016195 | CUGAAGAAGCUACUGCUUGTT | 453 |
| MPHOSPH1 | NM_016195 | GACAUGCGAAUGACACUAGTT | 454 |
| MPHOSPH1 | NM_016195 | AAGUUUGUGUCCCAGACACTT | 455 |
| MPHOSPH1 | NM_016195 | AAUGGCAGUGAAACACCCUTT | 456 |
| MPHOSPH1 | NM_016195 | AUGAAGGAGAGUGAUCACCTT | 457 |
| MYO3A | NM_017433 | AAAGCUACCGAUGUCAGGGTT | 458 |
| MYO3A | NM_017433 | AAAUCCCGAGUUAUCCACCTT | 459 |
| MYO3A | NM_017433 | GGCUAAUGAAAGGUGCUGGTT | 460 |
| NEK1 | AB067488 | AAGUGACAUUUGGGCUCUGTT | 461 |
| NEK1 | AB067488 | AUGCACGUGCUGCUGUACUTT | 462 |
| NEK1 | AB067488 | GAAGGACCUUCUGAUUCUGTT | 463 |
| NFKB2 | NM_002502 | AGGAUUCUCAUGGGAAGGGTT | 464 |
| NFKB2 | NM_002502 | GAAGAACAUGAUGGGGACUTT | 465 |
| NFKB2 | NM_002502 | GAUUGAGCGGCCUGUAACATT | 466 |
| NOTCH1 | AF308602 | AGGCAAGCCCUGCAAGAAUTT | 467 |
| NOTCH1 | AF308602 | AUAUCGACGAUUGUCCAGGTT | 468 |
| NOTCH1 | AF308602 | CACUUACACCUGUGUGUGCTT | 469 |
| NOTCH3 | NM_000435 | AAUGGCUUCCGCUGCCUCUTT | 470 |
| NOTCH3 | NM_000435 | GAACAUGGCCAAGGGUGAGTT | 471 |
| NOTCH3 | NM_000435 | GAGUCUGGGACCUCCUUCUTT | 472 |
| NOTCH4 | NM_004557 | CCAGCACUGACUACUGUGUTT | 473 |
| NOTCH4 | NM_004557 | GGAACUCGAUGCUUGUCAGTT | 474 |
| NOTCH4 | NM_004557 | UGCGAGGAAGAUACGGAGUTT | 475 |
| NR4A2 | NM_006186 | AAGGCCGGAGAGGUCGUUUTT | 476 |
| NR4A2 | NM_006186 | CAUCGACAUUUCUGCCUUCTT | 477 |
| NR4A2 | NM_006186 | GUCACAUGGGCAGAGAUAGTT | 478 |
| NRAS | NM_002524 | AAGAGCCACUUUCAAGCUGTT | 479 |
| NRAS | NM_002524 | AGUAGCAACUGCUGGUGAUTT | 480 |
| NRAS | NM_002524 | CCUCUACAGGGAGCAGAUUTT | 481 |
| PAK1 | NM_002576 | CCGCUGUCUCGAUAUGGAUTT | 482 |
| PAK1 | NM_002576 | GGACCGAUUUUACCGAUCCTT | 483 |
| PAK1 | NM_002576 | UGGAUGGCUCUGUCAAGCUTT | 484 |
| PDGFRB | NM_002609 | AAAGAAGUACCAGCAGGUGTT | 485 |
| PDGFRB | NM_002609 | UCCAUCCACCAGAGUCUAGTT | 486 |
| PDGFRB | NM_002609 | UUUGCUGAGCUCCAUCGGATT | 487 |
| PDZGEF2 | NM_016340 | AACCCUCAUCCACAGGUGATT | 488 |
| PDZGEF2 | NM_016340 | CCGACUGAGUACAUCGAUGTT | 489 |
| PDZGEF2 | NM_016340 | GCCAGAUUCGACUGAUUGUTT | 490 |
| PIK3C2A | NM_002645 | AACGAGGAAUCCGACAUUCTT | 491 |
| PIK3C2A | NM_002645 | UGAUGAGCCCAUCCUUUCATT | 492 |
| PIK3C2A | NM_002645 | UGCUUCAACGGAUGUAGCATT | 493 |
| PIK3CA | NM_006218 | AGGUGCACUGCAGUUCAACTT | 494 |
| PIK3CA | NM_006218 | UGGCUUUGAAUCUUUGGCCTT | 495 |
| PIK3CA | NM_006218 | UUCAGCUAGUACAGGUCCUTT | 496 |
| PIK3CB | NM_006219 | AAGUUCAUGUCAGGGCUGGTT | 497 |
| PIK3CB | NM_006219 | AAUGCGCAAAUUCAGCGAGTT | 498 |
| PIK3CB | NM_006219 | CAAAGAUGCCCUUCUGAACTT | 499 |
| PKD2 | NM_000297 | CGGCUAGUACGUGAAGAGUTT | 500 |
| PKD2 | NM_000297 | CUUAUAGUGGAGCUGGCUATT | 501 |
| PKD2 | NM_000297 | GAUAGCGGACAUAGCUCCATT | 502 |
| PLCG1 | NM_002660 | ACUACGUGGAAGAGAUGGUTT | 503 |
| PLCG1 | NM_002660 | AGGCAAGAAGUUCCUUCAGTT | 504 |
| PLCG1 | NM_002660 | GGAGAAUGGUGACCUCAGUTT | 505 |
| POLS | NM_006999 | ACAGAGACGCCGAAAGUACTT | 506 |
| POLS | NM_006999 | CUAGCGACAUAGACCUGGUTT | 507 |
| POLS | NM_006999 | GCAAAUGAAUUGGCCUGGCTT | 508 |
| PRKACB | NM_002731 | AAACCCUUGGAACAGGUUCTT | 509 |
| PRKACB | NM_002731 | CAAGAUGACAUCUGAGCUCTT | 510 |
| PRKACB | NM_002731 | UGUCUGAUCGAUCAUGCAGTT | 511 |
| PRKCL1 | NM_002741 | CACAAGAUCGUCUACAGGGTT | 512 |
| PRKCL1 | NM_002741 | CACCAGUGAAGUCAGCACUTT | 513 |
| PRKCL1 | NM_002741 | GAUUUCAAGUUCCUGGCGGTT | 514 |
| PRKCL2 | NM_006256 | AAGUGGCUCCUCAUUGUACTT | 515 |
| PRKCL2 | NM_006256 | AUCAUUCUGGCACCUUCAGTT | 516 |
| PRKCL2 | NM_006256 | GUAAAAGCGGAAGUAGUCGTT | 517 |
| PRKCQ | NM_006257 | AAAUGAAGCAAGGCCGCCATT | 518 |
| PRKCQ | NM_006257 | ACGGAGUACAGACGUCUCATT | 519 |
| PRKCQ | NM_006257 | GGAAGCAAAGGACCUUCUGTT | 520 |
| PRKG2 | NM_006259 | AAGACUGGAUCCUCAGCAGTT | 521 |
| PRKG2 | NM_006259 | CAAGUGCAUCCAGCUGAACTT | 522 |
| PRKG2 | NM_006259 | GAAAUUCCUGCACAAUGGGTT | 523 |
| PRKWNK3 | AJ409088 | ACCAAGCAGCCAGCUAUACTT | 524 |
| PRKWNK3 | AJ409088 | CUAAUGACAUCUGGGACCUTT | 525 |
| PRKWNK3 | AJ409088 | CUACGAAGGAAAACGUCAGTT | 526 |
| PRKY | NM_002760 | AGACAGUGAAGCUGGUUGUTT | 527 |
| PRKY | NM_002760 | GAAUUUCUGAGGACGAGCUTT | 528 |
| PRKY | NM_002760 | UCAGAUUUGGGCCAGAGUUTT | 529 |
| PTEN | NM_000314 | UGGAGGGGAAUGCUCAGAATT | 530 |
| PTEN | NM_000314 | AAGGCAGCUAAAGGAAGUGTT | 531 |
| PTEN | NM_000314 | UAAAGAUGGCACUUUCCCGTT | 532 |
| PTK2 | NM_005607 | CUUGGACGAUGUAUUGGAGTT | 533 |
| PTK2 | NM_005607 | GACUGAAAAUGCUUGGGCATT | 534 |
| PTK2 | NM_005607 | UCCCACACAUCUUGCUGACTT | 535 |
| PTK6 | NM_005975 | AACACCCUCUGCAAAGUUGTT | 536 |
| PTK6 | NM_005975 | CCGUGGUUCUUUGGCUGCATT | 537 |
| PTK6 | NM_005975 | UCAGGCUUAUCCGAUGUGCTT | 538 |
| RAB2 | NM_002865 | AAUUGGCCCUCAGCAUGCUTT | 539 |
| RAB2 | NM_002865 | GAAGGUGAAGCUUUUGCACTT | 540 |
| RAB2 | NM_002865 | GAGGUUUCAGCCAGUGCAUTT | 541 |
| RAD51L1 | NM_133510 | AACAGGACCGUACUGCUUGTT | 542 |
| RAD51L1 | NM_133510 | GAAGCCUUUGUUCAGGUCUTT | 543 |
| RAD51L1 | NM_133510 | GAGAGGCAUCCUCCUUGAATT | 544 |
| RAF1 | NM_002880 | CACUCUCUACCGAAGAUCATT | 545 |
| RAF1 | NM_002880 | GAUCCUAAAGGUUGUCGACTT | 546 |
| RAF1 | NM_002880 | GGAAGCCAUUUGCAGUGCUTT | 547 |
| RASAL2 | NM_004841 | AGUACCAGGAUUCUUCAGCTT | 548 |
| RASAL2 | NM_004841 | CUUAGUUCUGGGCCAUGUATT | 549 |
| RASAL2 | NM_004841 | GACCCCACUGACAGUGAUUTT | 550 |
| RASD1 | NM_016084 | CAAAACCAAGGAGAACGUGTT | 551 |
| RASD1 | NM_016084 | CCUAAGGAGGACCUUUUUGTT | 552 |
| RASD1 | NM_016084 | GAAACCGUCAUGCCCGCUUTT | 553 |
| RHOK | NM_002929 | AGUACACAGCAGGUUCAUCTT | 554 |
| RHOK | NM_002929 | CGUGAAUGAGGAGAACCCUTT | 555 |
| RHOK | NM_002929 | GUUUAAGGAGGGGCCUGUGTT | 556 |
| SOS1 | NM_005633 | AUUGACCACCAGGUUUCUGTT | 557 |
| SOS1 | NM_005633 | CUUACAAAAGGGAGCACACTT | 558 |
| SOS1 | NM_005633 | UAUCAGACCGGACCUCUAUTT | 559 |
| SRC | NM_005417 | CAAUUCGUCGGAGGCAUCATT | 560 |
| SRC | NM_005417 | GCAGUGCCUGCCUAUGAAATT | 561 |
| SRC | NM_005417 | GGGGAGUUUGCUGGACUUUTT | 562 |
| SRC | NM_005417 | GAACCGGAUGCAGUUGAGCTT | 563 |
| SRC | NM_005417 | GCCGGAAUACAAGAACGGGTT | 564 |
| SRC | NM_005417 | GUGGCUCUUAUCCGCAUGATT | 565 |
| SRPK1 | NM_003137 | CGCUUAUGGAACGUGAUACTT | 566 |
| SRPK1 | NM_003137 | GCAACAGAAUGGCAGCGAUTT | 567 |
| SRPK1 | NM_003137 | GUUCUAAUCGGAUCUGGCUTT | 568 |
| STAT2 | NM_005419 | AAAGCCUGCAUCAGAGCUCTT | 569 |
| STAT2 | NM_005419 | AGUUAAUCUCCAGGAACGGTT | 570 |
| STAT2 | NM_005419 | UUUGCUCAGCCCAAACCUUTT | 571 |
| STAT4 | NM_003151 | ACACAGAUCUGCCUCUAUGTT | 572 |
| STAT4 | NM_003151 | CCCUACAAUAAAGGCCGGUTT | 573 |
| STAT4 | NM_003151 | UUAGGAAGGUCCUUCAGGGTT | 574 |
| STK10 | NM_005990 | AGACCAGUACUUCCUCCAGTT | 575 |
| STK10 | NM_005990 | CCAUACUCAGAACUCCUCUTT | 576 |
| STK10 | NM_005990 | GAAAAAGCAUCAGGGGGAATT | 577 |
| STK38L | BC028603 | AGACACCUUGACAGAAGAGTT | 578 |
| STK38L | BC028603 | CUCUGGGGAUUUCUCAAUGTT | 579 |
| STK38L | BC028603 | GGAAGUAAAUGCAGGCCAGTT | 580 |
| STK6 | NM_003600 | ACAGUCUUAGGAAUCGUGCTT | 581 |
| STK6 | NM_003600 | GCACAAAAGCUUGUCUCCATT | 582 |
| STK6 | NM_003600 | UUGCAGAUUUUGGGUGGUCTT | 583 |
| STK6 | NM_003600 | CACCCAAAAGAGCAAGCAGTT | 584 |
| STK6 | NM_003600 | CCUCCCUAUUCAGAAAGCUTT | 585 |
| STK6 | NM_003600 | GACUUUGAAAUUGGUCGCCTT | 586 |
| STMN1 | NM_005563 | AACUGCACAGUGCUGUUGGTT | 587 |
| STMN1 | NM_005563 | UACCCAACGCACAAAUGACTT | 588 |
| STMN1 | NM_005563 | UGGCUAGUACUGUAUUGGCTT | 589 |
| SYK | NM_003177 | AGAACUGGGCUCUGGUAAUTT | 590 |
| SYK | NM_003177 | AGAAGUUCGACACGCUCUGTT | 591 |
| SYK | NM_003177 | GGAAAACCUCAUCAGGGAATT | 592 |
| TCF1 | NM_000545 | AGCCGUGGUGGAGACCCUUTT | 593 |
| TCF1 | NM_000545 | AGUCAAGGAGAAAUGCGGUTT | 594 |
| TCF1 | NM_000545 | CCUCGUCACGGAGGUGCGUTT | 595 |
| TGFBR1 | NM_004612 | GACAUGAUUCAGCCACAGATT | 596 |
| TGFBR1 | NM_004612 | UUCCUCGAGAUAGGCCGUUTT | 597 |
| TGFBR1 | NM_004612 | UUUGGGAGGUCAGUUGUUCTT | 598 |
| TGFBR2 | NM_003242 | CCAGAAAUCCUGCAUGAGCTT | 599 |
| TGFBR2 | NM_003242 | GCAGAACACUUCAGAGCAGTT | 600 |
| TIE | NM_005424 | AAAAAGGGAUCUGGGGAUGTT | 601 |
| TIE | NM_005424 | CGUGACGUUAAUGAACCUGTT | 602 |
| TIE | NM_005424 | GAGCAACGGAUCCUACUUCTT | 603 |
| TK1 | NM_003258 | AAGCACAGAGUUGAUGAGATT | 604 |
| TK1 | NM_003258 | CCUUGCUGGGACUUGGAUCTT | 605 |
| TK1 | NM_003258 | CGCCGGGAAGACCGUAAUUTT | 606 |
| TLE1 | NM_005077 | AGAUGACAAGAAGCACCACTT | 607 |
| TLE1 | NM_005077 | AGGAAGGUGGAUGAUAAGGTT | 608 |
| TLE1 | NM_005077 | CACCUGUUUCCAAACCUUGTT | 609 |
| TLK2 | NM_006852 | AGAGCUGGAUCAUCCCAGATT | 610 |
| TLK2 | NM_006852 | AGGCGUUUAUUCGACGAUGTT | 611 |
| ThK2 | NM_006852 | CACUUGACGGUUGUCCCUUTT | 612 |
| TOP3A | NM_004618 | GAUCCUCCCUGUCUAUGAGTT | 613 |
| TOP3A | NM_004618 | GCAAAGAAAUUGGACGAGGTT | 614 |
| TOP3A | NM_004618 | GGCGAAAACAUCGGGUUUGTT | 615 |
| TYRO3 | NM_006293 | GACUAACAAAGGCAGCUGUTT | 616 |
| TYRO3 | NM_006293 | GCAGCUUGCAUGAAGGAGUTT | 617 |
| TYRO3 | NM_006293 | UGCCCCUUUCCAACUGUCUTT | 618 |
| VHL | NM_000551 | AGGAAAUAGGCAGGGUGUGTT | 619 |
| VHL | NM_000551 | CAGAACCCAAAAGGGUAAGTT | 620 |
| VHL | NM_000551 | GAUCUGGAAGACCACCCAATT | 621 |
| WASL | NM_003941 | AAACAGGAGGUGUUGAAGCTT | 622 |
| WASL | NM_003941 | AAGUGGAGCAGAACAGUCGTT | 623 |
| WASL | NM_003941 | GGACAAUCCACAGAGAUCUTT | 624 |
| WEE1 | NM_003390 | AUCGGCUCUGGAGAAUUUGTT | 625 |
| WEE1 | NM_003390 | CAAGGAUCUCCAGUCCACATT | 626 |
| WEE1 | NM_003390 | UGUACCUGUGUGUCCAUCUTT | 627 |
| WNT1 | NM_005430 | ACGGCGUUUAUCUUCGCUATT | 628 |
| WNT1 | NM_005430 | CCCUCUUGCCAUCCUGAUGTT | 629 |
| WNT1 | NM_005430 | CUAUUUAUUGUGCUGGGUCTT | 630 |
| WNT2 | NM_003391 | AUUUGCCCGCGCAUUUGUGTT | 631 |
| WNT2 | NM_003391 | AACGGGCGAUUAUCUCUGGTT | 632 |
| WNT2 | NM_003391 | AGAAGAUGAAUGGUCUGGCTT | 633 |
| ZW10 | NM_004724 | ACAGUUGCAGGAGUUUUCCTT | 634 |
| ZW10 | NM_004724 | CAAACUGUCAGGCAGCAUUTT | 635 |
| ZW10 | NM_004724 | GCCAGCUUGCAAGAAAUUGTT | 636 |
| | XM_170783 | ACCGACACUUUGGCUUCCATT | 637 |
| | XM_170783 | GAUGAGCGCGGGAAUGUUGTT | 638 |
| | XM_170783 | UGGCCGAGGCCUUCAAGCUTT | 639 |
| | XM_064050 | CAUCAAUCACUCUCUGCUGTT | 640 |
| | XM_064050 | CUAACCCAGGAUGUUCAGGTT | 641 |
| | XM_064050 | GACACUCACCAUGCUGAAATT | 642 |
| | XM_066649 | AAGGGUGACUUUGUGUCCUTT | 643 |
| | XM_066649 | ACCAGGAACAAACCUGUUGTT | 644 |
| | XM_066649 | UUUGAAGGUGGCCCUCCUATT | 645 |
| | NM_005200 | AUGAAGCCUCACCAGGACUTT | 646 |
| | NM_005200 | CACUUUUCCCUCAACGAGGTT | 647 |
| | NM_005200 | UAGUAGCAAAGCAGGAAGGTT | 648 |
| | NM_139286 | GUUGUAGGAGGCAGUGAUGTT | 649 |
| | NM_139286 | UCUCAAUUUGGAAGUCUUGTT | 650 |
| | NM_139286 | UGAUCAAUGAUCGGAUUGGTT | 651 |
| | NM_013366 | CGAUCUGCAGGCCAACAUCTT | 652 |
| | NM_013366 | GAAGUAUGAGCAGCUCAAGTT | 653 |
| | NM_013366 | GGACCUCUUCAUCAAUGAGTT | 654 |
| | NM_014885 | ACCAGGAUUUGGAGUGGAUTT | 655 |
| | NM_014885 | CAAGGCAUCCGUUAUAUCUTT | 656 |
| | NM_014885 | GUGGCUGGAUUCAUGUUCCTT | 657 |
| | NM_016263 | CCAGAUCCUUGUCUGGAAGTT | 658 |
| | NM_016263 | CGACAACAAGCUGCUGGUCTT | 659 |
| | NM_016263 | GAAGCUGUCCAUGUUGGAGTT | 660 |
| | NM_013367 | AGCCAGCAGAUGUAAUUGGTT | 661 |
| | NM_013367 | CAUUUCAAUGAGGCUCCAGTT | 662 |
| | NM_013367 | GUCAUUUACAGAGUGGCUCTT | 663 |
| | NM_018492 | AGGACACUUUGGGUACCAGTT | 664 |
| | NM_018492 | GACCCUAAAGAUCGUCCUUTT | 665 |
| | NM_018492 | GCUGAGGAGAAUAUGCCUCTT | 666 |
| | NM_006087 | CGUGUACUACAACGAGGCCTT | 667 |
| | NM_006087 | UCCCCUCUGACUCCAACUUTT | 668 |
| | NM_006087 | CGAGGCACUCUACGACAUCTT | 669 |
| | NM_016231 | GCAAUGAGGACAGCUUGUGTT | 670 |
| | NM_016231 | UCUCCUUGUGAACAGCAACTT | 671 |
| | NM_016231 | UGUAGCUUUCCACUGGAGUTT | 672 |
| | XM_095827 | AAGGUCUUUACGCCAGUACTT | 673 |
| | XM_095827 | GGAAUGUAUCCGAGCACUGTT | 674 |
| | XM_095827 | UAAGCCUGGUGGUGAUCUUTT | 675 |
| | NM_145754 | CUCAAGGGAAAUAUCCGUGTT | 676 |
| | NM_145754 | GUGUGUUGUGCCUGCUGAATT | 677 |
| | NM_145754 | UCAGGCAUGGCAUUAAAACTT | 678 |
| | XM_168069 | CAAAGUUAUUAGCCCCAAGTT | 679 |
| | XM_168069 | CAGAGGCCAAGUAUAUCAATT | 680 |
| | XM_168069 | CCUGCAGAUUUGCACAGCGTT | 681 |
| | NM_021170 | AUCCUGGAGAUGACCGUGATT | 682 |
| | NM_021170 | GCCGGUCAUGGAGAAGCGGTT | 683 |
| | NM_021170 | UGGCCCUGAGACUGCAUCGTT | 684 |
| | NM_019089 | CCCCUCCAUGCUCAGAACUTT | 685 |
| | NM_019089 | CCUAUCUGGGAAGCCUGUGTT | 686 |
| | NM_019089 | UGCCCCAGUGACAAUAACATT | 687 |
| | NM_016653 | ACCAGGGCCAAAAUUAUGGTT | 688 |
| | NM_016653 | AUAGUGAACCUGGAACUGGTT | 689 |
| | NM_016653 | GCAGUUGCCCCAGAAGUUUTT | 690 |
| | NM_016281 | AGAACACACUGCUUGGUUGTT | 691 |
| | NM_016281 | GACAGUGAACAUGGAACCATT | 692 |
| | NM_016281 | GAGAACUUGCAGCACACACTT | 693 |
| | NM_012119 | ACCUGCCAACCUGCUCAUCTT | 694 |
| | NM_012119 | GAUCUCCUUUAAGGAGCAGTT | 695 |
| | NM_012119 | GCAGCUGUGUAUUUAAGGATT | 696 |
| | AB002301 | AGACAAAGAGGGGACCUUCTT | 697 |
| | AB002301 | GAAAGUCUAUCCGAAGGCUTT | 698 |
| | AB002301 | UGCCUCCCUGAAACUUCGATT | 699 |
| | NM_018401 | AGGUAUGCAUCGUGCAGAATT | 700 |
| | NM_018401 | GCAAUCAAACCGUCAUGACTT | 701 |
| | NM_018401 | UAUCCUGCUGGAUGAACACTT | 702 |
| | NM_006622 | GCAAGGUAUACAAUGCCGUTT | 703 |
| | NM_006622 | UAACUCAGCAACCCAGCAATT | 704 |
| | NM_006622 | UGCCUUGAAGACAGUACCATT | 705 |
| | A1278633 | CCUCAGCCGUAUAAUACGUTT | 706 |
| | A1278633 | CUGCUCUGUUCAAUCCCAGTT | 707 |
| | A1278633 | CUGGGAUUGGCCACCUCUUTT | 708 |
| | NM_152524 | AGAAGGAGAGUGUCAGGUUTT | 709 |
| | NM_152524 | UAUGUACCCCGUUCAGCAATT | 710 |
| | NM_152524 | UUUUGCCUUGGAGUGCUCCTT | 711 |
| | NM_019013 | AAAACCCCCGGGAGUCGUCTT | 712 |
| | NM_019013 | AGUGGCACCAAGUGGCUGGTT | 713 |
| | NM_019013 | GAAACCUGCUUUGUCAUUUTT | 714 |
| | A1338451 | CUGAUGCACUUUGCUGCAGTT | 715 |
| | A1338451 | CUGCAGGUUCAAAUCCCAGTT | 716 |
| | A1338451 | GGGGAAAAAGCUUUGCGUUTT | 717 |
| | NM_018410 | AAAGACCCAGGCUAUCAGATT | 718 |
| | NM_018410 | CAGACCCCAAAUCCAUAAGTT | 719 |
| | NM_018410 | GUCAGUGUCACCCAGCAAATT | 720 |
| | NM_018123 | UAUCGAGCCACCAUUUGUGTT | 721 |
| | NM_018123 | UGAUGCAUAUAGCCGCAACTT | 722 |
| | NM_018123 | UGCACAGGGCCAAAGUUGATT | 723 |

TABLE IIIC

siRNA sequences used in screens of DNA damaging
agents: camptothecin screen

| | | | |
|---|---|---|---|
| GENE | | | SEQ ID |
| SYMBOL | SEQUENCE_ID | SENSE_SEQ | NO |

| | | | |
|---|---|---|---|
| AATK | AB014541 | CGCAAGAAGAAGGCCGUGUTT | 724 |
| AATK | AB014541 | CGCUGGUGCAAUGUUUUCUTT | 725 |
| AATK | AB014541 | GAAUCCCUACCGAGACUCUTT | 726 |
| ABL1 | NM_007313 | AAACCUCUACACGUUCUGCTT | 727 |
| ABL1 | NM_007313 | CUAAAGGUCAAAAGCUCCGTT | 728 |
| ABL1 | NM_007313 | UCCUGGCAAGAAAGCUUGATT | 729 |
| ABL2 | NM_007314 | AUCAGUGAUGUGGUGCAGATT | 730 |
| ABL2 | NM_007314 | GACUCGGACACUGAAGAAATT | 731 |
| ABL2 | NM_007314 | UGGCACAGCAGGUACUAAATT | 732 |
| ACVR2 | NM_001616 | AAGAUGGCCACAAACCUGCTT | 733 |
| ACVR2 | NM_001616 | AGAUAAACGGCGGCAUUGUTT | 734 |
| ACVR2 | NM_001616 | GACAUGCAGGAAGUUGUUGTT | 735 |
| ACVR2B | NM_001106 | CGGGAGAUCUUCAGCACACTT | 736 |
| ACVR2B | NM_001106 | GAGAUUGGCCAGCACCCUUTT | 737 |
| ACVR2B | NM_001106 | GCCCAGGACAUGAGUGUCUTT | 738 |
| AKT2 | NM_001626 | AGAUGGCCACAUCAAGAUCTT | 739 |
| AKT2 | NM_001626 | GUCAUCAUUGCCAAGGAUGTT | 740 |
| AKT2 | NM_001626 | UGCCAGCUGAUGAAGACCGTT | 741 |
| ANAPC5 | NM_016237 | ACAGUGCUGAACUUGGCUUTT | 742 |
| ANAPC5 | NM_016237 | CCAAAUGUCAGAGGCACAUTT | 743 |
| ANAPC5 | NM_016237 | UCAAACUGAUGGCUGAAGGTT | 744 |
| AXIN1 | AF009674 | GAAAGUGAGCGACGAGUUUTT | 745 |
| AXIN1 | AF009674 | GUGCCUUCAACACAGCUUGTT | 746 |
| AXIN1 | AF009674 | UGAAUAUCCAAGAGCAGGGTT | 747 |
| BCL2 | NM_000633 | AGGACAUUUGUUGGAGGGGTT | 748 |
| BCL2 | NM_000633 | UCUACCAAUUGUGCCGAGATT | 749 |
| BCL2 | NM_000633 | UGAAGAACGUGGACGCUUUTT | 750 |
| BLK | NM_001715 | AGUCACGAGCGUUCGAAAATT | 751 |
| BLK | NM_001715 | CAACAUGAAGGUGGCCAUUTT | 752 |
| BLK | NM_001715 | GCACUAUAAGAUCCGCUGCTT | 753 |
| BMPR1B | NM_001203 | ACAGAUUGGAAAAGGUCGCTT | 754 |
| BMPR1B | NM_001203 | GAAGUUACGCCCCUCAUUCTT | 755 |
| BMPR1B | NM_001203 | UAUUUGCAGCACAGACGGATT | 756 |
| BMPR2 | NM_001204 | CAAAUCUGUGAGCCCAACATT | 757 |
| BMPR2 | NM_001204 | CAAGAUGUUCUUGCACAGGTT | 758 |
| BMPR2 | NM_001204 | GAACGGCUAUGUGCGUUUATT | 759 |
| BRCA1 | NM_007296 | ACUUAGGUGAAGCAGCAUCTT | 760 |
| BRCA1 | NM_007296 | GGGCAGUGAAGACUUGAUUTT | 761 |
| BRCA1 | NM_007296 | UGAAGUGGGCUCCAGUAUUTT | 762 |
| BRCA2 | NM_000059 | CAAAUGGGCAGGACUCUUATT | 763 |
| BRCA2 | NM_000059 | CUGUUCAGCCCAGUUUGAATT | 764 |
| BRCA2 | NM_000059 | UCUCCAAGGAAGUUGUACCTT | 765 |
| C20orf97 | NM_021158 | AGUCCCAGGUGGGACUCUUTT | 766 |
| C20orf97 | NM_021158 | CUGGCAUCCUUGAGCUGACTT | 767 |
| C20orf97 | NM_021158 | GACUGUUCUGGAAUGAGGGTT | 768 |
| CAMK2D | NM_001221 | ACCAGAUGGAGUAAAGGAGTT | 769 |
| CAMK2D | NM_001221 | GCACCCUAAUAUUGUGCGATT | 770 |
| CAMK2D | NM_001221 | UUGGCAGACUUUGGCUUAGTT | 771 |
| CCND1 | NM_053056 | CAUGUAACCGGCAUGUUUCTT | 772 |
| CCND1 | NM_053056 | CCCACAGCUACUUGGUUUGTT | 773 |
| CCND1 | NM_053056 | UGACCCCGCACGAUUUCAUTT | 774 |
| CCNE2 | NM_057749 | CCACAGAUGAGGUCCAUACTT | 775 |
| CCNE2 | NM_057749 | CUGGGGCUUUCUUGACAUGTT | 776 |
| CCNE2 | NM_057749 | GUGGUUAAGAAAGCCUCAGTT | 777 |
| CCNT2 | NM_058241 | AGCGCCAGUAAAGAAGAACTT | 778 |
| CCNT2 | NM_058241 | AGGGCAGCCAGUUGUCAUUTT | 779 |
| CCNT2 | NM_058241 | CCACCACUCCAAAAUGAGCTT | 780 |
| CDC14B | NM_033331 | GGCCAUCCCCUCCAUUAAUTT | 781 |
| CDC14B | NM_033331 | GUAAUUGAAAGGCAGUGCCTT | 782 |
| CDC14B | NM_033331 | UUGCUAUCACUGUGGCUCUTT | 783 |
| CDC16 | NM_003903 | AGUGGCUUCAAAGAUCCCUTT | 784 |
| CDC16 | NM_003903 | GCAUGUCGUUACCUUGCAGTT | 785 |
| CDC16 | NM_003903 | UAAGCCUAGUGAAACGGUCTT | 786 |
| CDC23 | NM_004661 | AGCAACUGCUGCUUAUUGCTT | 787 |
| CDC23 | NM_004661 | AGCAAGCAAGGAGAUAGGATT | 788 |
| CDC23 | NM_004661 | CCUUCUUUAUGUCAGGAGCTT | 789 |
| CDC25B | NM_021874 | AGGAUGAUGAUGCAGUUCCTT | 790 |
| CDC25B | NM_021874 | GACAAGGAGAAUGUGCGCUTT | 791 |
| CDC25B | NM_021874 | GAGCCCAGUCUGUUGAGUUTT | 792 |
| CDC34 | NM_004359 | ACGUGGACGCCUCCGUGAUTT | 793 |
| CDC34 | NM_004359 | CACCUACUACGAGGGCGGCTT | 794 |
| CDC34 | NM_004359 | CAUCUACGAGACGGGGGACTT | 795 |
| CDC37 | NM_007065 | CGCAUGGAGCAGUUCCAGATT | 796 |
| CDC37 | NM_007065 | GACGGCUUCAGCAAGAGCATT | 797 |
| CDC37 | NM_007065 | GAUUAAGACAGCCGAUCGCTT | 798 |
| CDC42 | NM_044472 | ACCUUAUGGAAAAGGGGUGTT | 799 |
| CDC42 | NM_044472 | CCAUCCUGUUUGAAAGCCUTT | 800 |
| CDC42 | NM_044472 | CCCAAAAGGAAGUGCUGUATT | 801 |
| CDC45L | NM_003504 | CACCUGCUCAAGUCCUUUGTT | 802 |
| CDC45L | NM_003504 | GAUCCUUCAGGCCUUGUUCTT | 803 |
| CDC45L | NM_003504 | UGACAGUGAUGGGUCAGAGTT | 804 |
| CDK2 | NM_001798 | AUGAUAGCGGGGGCUAAGUTT | 805 |
| CDK2 | NM_001798 | GAGCUAUCUGUUCCAGCUGTT | 806 |
| CDK2 | NM_001798 | UCUAUUGCUUCACCAUGGCTT | 807 |
| CDK2AP1 | NM_004642 | AGCAAAUACGCGGAGCUGCTT | 808 |
| CDK2AP1 | NM_004642 | CUGCCCAGGUUUUUUUUGUTT | 809 |
| CDK2AP1 | NM_004642 | GUUACAGUUCAUCUCCCCUTT | 810 |
| CDK4 | NM_000075 | CCCUGGUGUUUGAGCAUGUTT | 811 |
| CDK4 | NM_000075 | CUGACCGGGAGAUCAAGGUTT | 812 |
| CDK4 | NM_000075 | GAGUGUGAGAGUCCCCAAUTT | 813 |
| CDK5R2 | NM_003936 | AGGCGAGAGCCGACUCAAGTT | 814 |
| CDK5R2 | NM_003936 | CCUGGACCGCUAGGGAUACTT | 815 |
| CDK5R2 | NM_003936 | CGCAACCGCGAGAACCUUCTT | 816 |
| CDK7 | NM_001799 | AACUGGCAGAUUUUGGCCUTT | 817 |
| CDK7 | NM_001799 | CUGUCCAGUGGAAACCUUATT | 818 |
| CDK7 | NM_001799 | UAGAACCGCCUUAAGAGAGTT | 819 |
| CDKL5 | NM_003159 | ACAGUACCCAAUUCCGACATT | 820 |
| CDKL5 | NM_003159 | GGAGAAUACUUCUGCUGUGTT | 821 |
| CDKL5 | NM_003159 | UCAGCCACAAUGAUGUCCUTT | 822 |
| CDKN1A | NM_078467 | AACUAGGCGGUUGAAUGAGTT | 823 |
| CDKN1A | NM_078467 | CAUACUGGCCUGGACUGUUTT | 824 |
| CDKN1A | NM_078467 | GAUGGUGGCAGUAGAGGCUTT | 825 |
| CHEK1 | NM_001274 | AUCGAUUCUGCUCCUCUAGTT | 826 |
| CHEK1 | NM_001274 | CUGAAGAAGCAGUCGCAGUTT | 827 |
| CHEK1 | NM_001274 | UGCCUGAAAGAGACUUGUGTT | 828 |
| CHEK1 | NM_001274 | CCAGUUGAUGUUUGGUCCUTT | 829 |
| CHEK1 | NM_001274 | UCUCAGACUUUGGCUUGGCTT | 830 |
| CHEK1 | NM_001274 | UUCUAUGGUCACAGGAGAGTT | 831 |
| CHFR | NM_018223 | AGACUGCGUCCUUUUCGUCTT | 832 |
| CHFR | NM_018223 | GAUACCAGCACCAGUGGAATT | 833 |
| CHFR | NM_018223 | GCAUACCUCAUCCAGCAUCTT | 834 |
| CKAP2 | NM_018204 | CCAAUCACAAGUCCuAUUGTT | 835 |
| CKAP2 | NM_018204 | CUUGUGCGACCUCCUAUUATT | 836 |
| CKAP2 | NM_018204 | GAGAGAAAAGCUCGUCUGATT | 837 |
| CREBBP | NM_004380 | AUUUUUGCGGCGCCAGAAUTT | 838 |
| CREBBP | NM_004380 | GAAAAACGGAGGUCGCGUUTT | 839 |
| CREBBP | NM_004380 | GAAAACAAAUGCCCCGUGCTT | 840 |
| CSF1R | NM_005211 | AGUGCAGAAAGUCAUCCCATT | 841 |
| CSF1R | NM_005211 | CAACCUGCAGUUUGGUAAGTT | 842 |
| CSF1R | NM_005211 | UGAGCCAAGUGGCAGCUAATT | 843 |
| CTNNA1 | NM_001903 | CGUUCCGAUCCUCUAUACUTT | 844 |
| CTNNA1 | NM_001903 | UGACAUCAUUGUGCUGGCCTT | 845 |
| CTNNA1 | NM_001903 | UGACCAAAGAUGACCUGUGTT | 846 |
| CTNNAL1 | NM_003798 | AAGUGUUGUUGCUGGCAGATT | 847 |
| CTNNAL1 | NM_003798 | AACUGAGAAGCUUUUGGGGTT | 848 |
| CTNNAL1 | NM_003798 | CUAGAGGUUUUUGCUGCAGTT | 849 |
| CTNNBIP1 | NM_020248 | AAAUUUGCGCCUCGGUAUCTT | 850 |
| CTNNBIP1 | NM_020248 | ACCUAAGUCCUUCCACCUGTT | 851 |
| CTNNB1P1 | NM_020248 | CACCCUGGAUGCUGUUGAATT | 852 |
| CUL1 | NM_003592 | GACCGCAAACUACUGAUUCTT | 853 |
| CUL1 | NM_003592 | GCCAGCAUGAUCUCCAAGUTT | 854 |
| CUL1 | NM_003592 | UAGACAUUGGGUUCGCCGUTT | 855 |
| DAPK2 | NM_014326 | GAAUAUUUUUGGGACGCCGTT | 856 |
| DAPK2 | NM_014326 | UCCAAGAGGCUCUCAGACATT | 857 |
| DAPK2 | NM_014326 | UCUCAGAAGGUCCUCCUGATT | 858 |
| DCC | NM_005215 | ACAUCGUGGUGCGAGGUUATT | 859 |
| DCC | NM_005215 | AUGAGCCGCCAAUUGGACATT | 860 |
| DCC | NM_005215 | AUGGCAAGUUUGGAAGGACTT | 861 |
| DDR1 | NM_013994 | AACAAGAGGACACAAUGGCTT | 862 |
| DDR1 | NM_013994 | AGAGGUGAAGAUCAUGUCGTT | 863 |
| DDR1 | NM_013994 | UCGCAGACUUUGGCAUGAGTT | 864 |
| DMPK | NM_004409 | CAAGUGGGACAUGCUGAAGTT | 865 |
| DMPK | NM_004409 | UAAAAGGCCCUCCAUCUGCTT | 866 |
| DMPK | NM_004409 | UUGGCCCUGUUCAGCAAUGTT | 867 |
| DTX1 | NM_004416 | AACCCACCUGAUGAGGACUTT | 868 |
| DTX1 | NM_004416 | GACCGAGUUUGGAUCCAACTT | 869 |
| DTX1 | NM_004416 | GAUGGAGUUCCACCUCAUCTT | 870 |
| DYRK3 | NM_003582 | CCAUGUUUGCAUGGCCUUUTT | 871 |
| DYRK3 | NM_003582 | CUUCUGGAGCAAUCCAAACTT | 872 |
| DYRK3 | NM_003582 | UCUUUGGAUGCCCUCCACATT | 873 |
| ECU | NM_018098 | ACUGGCUAAAGAUGCUGUGTT | 874 |
| ECU | NM_018098 | GACCAUGGGAAAAUUGUGGTT | 875 |
| ECU | NM_018098 | GCUUAGUACAGCGGGUUGATT | 876 |
| EGR2 | NM_000399 | CACUACCACCCUUUCCUGUTT | 877 |
| EGR2 | NM_000399 | GUGCAAUGUGAUGGGAGGATT | 878 |
| EGR2 | NM_000399 | UGUUACCGGAGCUGAUUUGTT | 879 |
| ELK1 | NM_005229 | GCCAUUCCUUUGUCUGCCATT | 880 |
| ELK1 | NM_005229 | GUGAAAGUAGAAGGGCCCATT | 881 |
| ELK1 | NM_005229 | UUCAAGCUGGUGGAUGCAGTT | 882 |
| ELK1 | NM_005229 | AGGACCCUUUCAAUGUCCCTT | 883 |
| ELK1 | NM_005229 | CUCUCAUUAUCUCCUCCACTT | 884 |
| ELK1 | NM_005229 | GCUCUCCUUCCAGUUUCCATT | 885 |
| EPHA4 | NM_004438 | CUGGCUACGAACUGAUUGGTT | 886 |
| EPHA4 | NM_004438 | GAUUCCUAUCCGGUGGACUTT | 887 |
| EPHA4 | NM_004438 | GCUAUCGUAUAGUUCGGACTT | 888 |
| EPHB3 | NM_004443 | GAAGAUCCUGAGCAGUAUCTT | 889 |
| EPHB3 | NM_004443 | GCUGCAGCAGUACAUUGCUTT | 890 |
| EPHB3 | NM_004443 | UACCCUGGACAAGCUCAUCTT | 891 |
| ETS1 | NM_005238 | UUCAGCCUGAAAGGUGUAGTT | 892 |
| ETS1 | NM_005238 | ACGCUACGUGUACCGCUUUTT | 893 |
| ETS1 | NM_005238 | UGACUACCCCUCGGUCAUUTT | 894 |
| FLT1 | NM_002019 | ACAUCGAAAACAGCAGGUGTT | 895 |
| FLT1 | NM_002019 | AGGAGGAGUGCAUCUUUGGTT | 896 |
| FLT1 | NM_002019 | UGGAUGAGGACUUUUGCAGTT | 897 |
| FOXO1A | NM_002015 | CUAUGCGUACUGCAUAGCATT | 898 |
| FOXO1A | NM_002015 | GACAACGACACAUAGCUGGTT | 899 |
| FOXO1A | NM_002015 | UACAAGGAACCUCAGAGCCTT | 900 |
| FRAT1 | NM_005479 | AAGCUAAUGACGAGGAACCTT | 901 |
| FRAT1 | NM_005479 | CCAUGGUGAAGUGCUUGGATT | 902 |
| FRAT1 | NM_005479 | UAACAGCUGCAAUUCCCUGTT | 903 |
| FRK | NM_002031 | ACUAUAGACUUCCGCAACCTT | 904 |
| FRK | NM_002031 | CAGUAGAUUGCUGUGGCCUTT | 905 |
| FRK | NM_002031 | CUCCAUACAGCUUCUGAAGTT | 906 |
| FZD9 | NM_003508 | GACUUUCCAGACCUGGCAGTT | 907 |
| FZD9 | NM_003508 | GAUCGGGGUCUUCUCCAUCTT | 908 |
| FZD9 | NM_003508 | GGACUUCGCGCUGGUCUGGTT | 909 |
| GPRK6 | NM_002082 | AAGCAAGAAAUGGCGGCAGTT | 910 |
| GPRK6 | NM_002082 | GAGCUGAAUGUCUUUGGGCTT | 911 |
| GPRK6 | NM_002082 | UGUAUAUAGCGACCAGAGCTT | 912 |
| GUK1 | NM_000858 | CGGCAAAGAUUACUACUUUTT | 913 |
| GUK1 | NM_000858 | GGAGCCCGGCCUGUUUGAUTT | 914 |
| GUK1 | NM_000858 | UCAAGAAAGCUCAAAGGACTT | 915 |
| HDAC3 | NM_003883 | CCCAGAGAUUUUUGAGGGATT | 916 |
| HDAC3 | NM_003883 | UGCCUUCAACGUAGGCGAUTT | 917 |
| HDAC3 | NM_003883 | UGGUACCUAUUAGGGAUGGTT | 918 |
| HDAC4 | NM_006037 | AGAGGACGUUUUCUACGGCTT | 919 |
| HDAC4 | NM_006037 | AUCUGUUUGCAAGGGGAAGTT | 920 |
| HDAC4 | NM_006037 | CAAGAUCAUCCCCAAGCCATT | 921 |
| HDAC5 | NM_005474 | AAACUGUUCUCAGAUGCCCTT | 922 |
| HDAC5 | NM_005474 | CCCAACUUGAAAGUGCGUUTT | 923 |
| HDAC5 | NM_005474 | UGAGAUGCACUCCUCCAGUTT | 924 |
| HDAC9 | NM_058176 | AAGCUUCUUGUAGCUGGUGTT | 925 |
| HDAC9 | NM_058176 | AUAUUGCCUGGACAGGUGGTT | 926 |
| HDAC9 | NM_058176 | CAGCAACAAGAACUCCUAGTT | 927 |
| HSPCB | NM_007355 | AGCAUUCAUGGAGGCUCUUTT | 928 |
| HSPCB | NM_007355 | AUUGACAUCAUCCCCAACCTT | 929 |
| HSPCB | NM_007355 | CUCAGCUUUUGUGGAGCGATT | 930 |
| IRS1 | NM_005544 | AGGGCAGUGGAGACUAUAUTT | 931 |
| IRS1 | NM_005544 | CCAGAGUGCCAAAGUGAUCTT | 932 |
| IRS1 | NM_005544 | GGAUAUAUUUGGCUGGGUGTT | 933 |
| KIF17 | XM_027915 | GAUAACGGCUUCUGGAAGATT | 934 |
| KIF17 | XM_027915 | GCAAAAGCAACUUUGGCAGTT | 935 |
| KIF17 | XM_027915 | GCUCAAUAUCAGCUGGGAATT | 936 |
| KIF25 | NM_005355 | GAGCUAUACCAUGCUGGGATT | 937 |
| KIF25 | NM_005355 | GGAUGGACGGACAGAGGUUTT | 938 |
| KIF25 | NM_005355 | GUUACUGGUGAUUCUCUGCTT | 939 |
| KIF26A | XM_050278 | AUGCGGAAUUUGCCGUGGGTT | 940 |
| KIF26A | XM_050278 | GCACAAGCACCUGUGUGAGTT | 941 |
| KIF26A | XM_050278 | GUCGUACACCAUGAUCGGGTT | 942 |
| KIF2C | NM_006845 | ACAAAAACGGAGAUCCGUCTT | 943 |
| KIF2C | NM_006845 | AUAAGCAGCAAGAAACGGCTT | 944 |
| KIF2C | NM_006845 | GAAUUUCGGGCUACUUUGGTT | 945 |
| KIF3B | NM_004798 | AAACGGUCCAUUGGUAGGATT | 946 |
| KIF3B | NM_004798 | AAGUGGAAGGAAGUCGGGATT | 947 |
| KIF3B | NM_004798 | UGCCAAGCAGUUUGAACUGTT | 948 |
| KIF4B | AF241316 | CCUGCAGCAACUGAUUACCTT | 949 |
| KIF4B | AF241316 | GAACUUGAGAAGAUGCGAGTT | 950 |
| KIF4B | AF241316 | GAAGAGGCCCACUGAAGUUTT | 951 |
| KRAS2 | NM_033360 | GAAAAGACUCCUGGCUGUGTT | 952 |
| KRAS2 | NM_033360 | GGACUCUGAAGAUGUACCUTT | 953 |
| KRAS2 | NM_033360 | GGCAUACUAGUACAAGUGGTT | 954 |
| LATS2 | NM_014572 | AACAGCCAUCCAAGUCUUCTT | 955 |
| LATS2 | NM_014572 | AACCUACCAGCAGAAGGUUTT | 956 |
| LATS2 | NM_014572 | UAGGCUUUUCAGGACCUUCTT | 957 |
| MAP2K7 | NM_005043 | AGUCCUACAGGAAGAGCCCTT | 958 |
| MAP2K7 | NM_005043 | GCUACUUGAACACAGCUUCTT | 959 |
| MAP2K7 | NM_005043 | UCAACGACCUGGAGAACUUTT | 960 |
| MAP3K1 | AF042838 | UCACUUAGCAGCUGAGUCUTT | 961 |
| MAP3K1 | AF042838 | UUGACAGCACUGGUCAGAGTT | 962 |
| MAP3K1 | AF042838 | UUGGCAAGAACUUCUUGGCTT | 963 |
| MAP3K4 | NM_005922 | AGAACGAUCGUCCAGUGGATT | 964 |
| MAP3K4 | NM_005922 | GGUACCUCGAUGCCAUAGUTT | 965 |
| MAP3K4 | NM_005922 | UUUUGGACUAGUGCGGAUGTT | 966 |
| MAP4K5 | NM_006575 | AAGGCUGCCACAAAUGUUGTT | 967 |
| MAP4K5 | NM_006575 | GAAACAGAAGCACGAGAUGTT | 968 |
| MAP4K5 | NM_006575 | UCUCUACAUCUUGGCUGGATT | 969 |
| MAPK13 | NM_002754 | CUCACAGUGGAUGAAUGGATT | 970 |
| MAPK13 | NM_002754 | GAUCAUGGGGAUGGAGUUCTT | 971 |
| MAPK13 | NM_002754 | UACAGCCUUUCAAGCAGAGTT | 972 |
| MAPK8 | NM_139049 | CACCCGUACAUCAAUGUCUTT | 973 |
| MAPK8 | NM_139049 | GGAAUAGUAUGCGCAGCUUTT | 974 |
| MAPK8 | NM_139049 | GUGAUUCAGAUGGAGCUAGTT | 975 |
| MAPRE1 | NM_012325 | GAGUAUUAACAGCCUGGACTT | 976 |
| MAPRE1 | NM_012325 | GCUAAGCUAGAACACGAGUTT | 977 |
| MAPRE1 | NM_012325 | UAGAGGAUGUGUUUCAGCCTT | 978 |
| MAPRE3 | NM_012326 | CAGCUUUGUUCAGGGGCAGTT | 979 |
| MAPRE3 | NM_012326 | CUUCGUGACAUCGAGCUCATT | 980 |
| MAPRE3 | NM_012326 | GGAUUACAACCCUCUGCUGTT | 981 |
| MARK1 | NM_018650 | ACAACAGCACUCUUCAGUCTT | 982 |
| MARK1 | NM_018650 | CUGCGAGAGCGAGUUUUACTT | 983 |
| MARK1 | NM_018650 | UGUGUAUUCUGGAGGUAGCTT | 984 |
| MCC | NM_002387 | AGUUGAGGAGGUUUCUGCATT | 985 |
| MCC | NM_002387 | GACUUAGAGCUGGGAAUCUTT | 986 |
| MCC | NM_002387 | GGAUUAUAUCCAGCAGCUCTT | 987 |
| MCM3 | NM_002388 | AGGAUUUUGUGGCCUCCAUTT | 988 |
| MCM3 | NM_002388 | GUCUCAGCUUCUGCGGUAUTT | 989 |
| MCM3 | NM_002388 | UCCAGGUUGAAGGCAUUCATT | 990 |
| MCM3 | NM_002388 | GCAGAUGAGCAAGGAUGCUTT | 991 |
| MCM3 | NM_002388 | GUACAUCCAUGUGGCCAAATT | 992 |
| MCM3 | NM_002388 | UGGGUCAUGAAAGCUGCCATT | 993 |
| MLH1 | NM_000249 | AACUGAAAGCCCCUCCUAATT | 994 |
| MLH1 | NM_000249 | GAUGGAAAUAUCCUGCAGCTT | 995 |
| MLH1 | NM_000249 | UGCUGUUAGUCGAGAACUGTT | 996 |
| MYB | NM_005375 | ACAAGAGGUGGAAUCUCCATT | 997 |
| MYB | NM_005375 | GGUUAUCUGCAGGAGUCUUTT | 998 |
| MYB | NM_005375 | UCGAACAGAUGUGCAGUGCTT | 999 |
| MYO3A | NM_017433 | AAAGCUACCGAUGUCAGGGTT | 1000 |
| MYO3A | NM_017433 | AAAUCCCGAGUUAUCCACCTT | 1001 |
| MYO3A | NM_017433 | GGCUAAUGAAAGGUGCUGGTT | 1002 |
| NEK1 | AB067488 | AAGUGACAUUUGGGCUCUGTT | 1003 |
| NEK1 | AB067488 | AUGCACGUGCUGCUGUACUTT | 1004 |
| NEK1 | AB067488 | GAAGGACCUUCUGAUUCUGTT | 1005 |
| NF1 | NM_000267 | AUCCUUCAACAAGGCACAGTT | 1006 |
| NF1 | NM_000267 | GUAACUUCAGCAGAGCGAATT | 1007 |
| NF1 | NM_000267 | UACAUGACUCCAUGGCUGUTT | 1008 |
| NFKB2 | NM_002502 | AGGAUUCUCAUGGGAAGGGTT | 1009 |
| NFKB2 | NM_002502 | GAAGAACAUGAUGGGGACUTT | 1010 |
| NFKB2 | NM_002502 | GAUUGAGCGGCCUGUAACATT | 1011 |
| NTRK1 | NM_002529 | CAACGGCAACUACACGCUGTT | 1012 |
| NTRK1 | NM_002529 | CGCCACAGCAUCAAGGAUGTT | 1013 |
| NTRK1 | NM_002529 | GAGUGGUCUCCGUUUCGUGTT | 1014 |
| OSR1 | NM_005109 | GAUACACAAAGAUGGGCUGTT | 1015 |
| OSR1 | NM_005109 | AAACAGCUCAGGCUUUGUCTT | 1016 |
| OSR1 | NM_005109 | GAAUAGUGGCUUACCGCUUTT | 1017 |
| PAK1 | NM_002576 | CCGCUGUCUCGAUAUGGAUTT | 1018 |
| PAK1 | NM_002576 | GGACCGAUUUUACCGAUCCTT | 1019 |
| PAK1 | NM_002576 | UGGAUGGCUCUGUCAAGCUTT | 1020 |
| PCNA | NM_002592 | AAUUGCGGAUAUGGGACACTT | 1021 |
| PCNA | NM_002592 | AGUCCAAAGUCUGAUCUGGTT | 1022 |
| PCNA | NM_002592 | UUUCCUGUGCAAAAGACGGTT | 1023 |
| PDGFRB | NM_002609 | AAAGAAGUACCAGCAGGUGTT | 1024 |
| PDGFRB | NM_002609 | UCCAUCCACCAGAGUCUAGTT | 1025 |
| PDGFRB | NM_002609 | UUUGCUGAGCUGCAUCGGATT | 1026 |
| PDZGEF2 | NM_016340 | AACCCUCAUCCACAGGUGATT | 1027 |
| PDZGEF2 | NM_016340 | CCGACUGAGUACAUCGAUGTT | 1028 |
| PDZGEF2 | NM_016340 | GCCAGAUUCGACUGAUUGUTT | 1029 |
| PIK3C2A | NM_002645 | AACGAGGAAUCCGACAUUCTT | 1030 |
| PIK3C2A | NM_002645 | UGAUGAGCCCAUCCUUUCATT | 1031 |
| PIK3C2A | NM_002645 | UGCUUCAACGGAUGUAGCATT | 1032 |
| POLS | NM_006999 | ACAGAGACGCCGAAAGUACTT | 1033 |
| POLS | NM_006999 | CUAGCGACAUAGACCUGGUTT | 1034 |
| POLS | NM_006999 | GCAAAUGAAUUGGCCUGGCTT | 1035 |
| PPARG | NM_015869 | AAUGACAGACCUCAGACAGTT | 1036 |
| PPARG | NM_015869 | UAAGCCUCAUGAAGAGCCUTT | 1037 |
| PPARG | NM_015869 | UGUCAGUACUGUCGGUUUCTT | 1038 |
| PRC1 | NM_003981 | AAGCAUAUCCGUCUGUCAGTT | 1039 |
| PRC1 | NM_003981 | AGGCUUCCAAAUCUGAUGCTT | 1040 |
| PRC1 | NM_003981 | GGAACUCUUUGAAGGUGUCTT | 1041 |
| PRKACA | NM_002730 | GAAUGGGGUCAACGAUAUCTT | 1042 |
| PRKACA | NM_002730 | GGACGAGACUUCCUCUUGATT | 1043 |
| PRKACA | NM_002730 | GUGUGGCAAGGAGUUUUCUTT | 1044 |
| PRKCB1 | NM_002738 | AGAGCAUGCAUUUUUCCGGTT | 1045 |
| PRKCB1 | NM_002738 | GGAGCCCCAUGCUGUAUUUTT | 1046 |
| PRKCB1 | NM_002738 | UUGGAUGUUAGCGGUACUCTT | 1047 |
| PRKCL1 | NM_002741 | CACAAGAUCGUCUACAGGGTT | 1048 |
| PRKCL1 | NM_002741 | CACCAGUGAAGUCAGCACUTT | 1049 |
| PRKCL1 | NM_002741 | GAUUUCAAGUUCCUGGCGGTT | 1050 |
| PRKCM | NM_002742 | AAUGAAUGAGGAGGGUAGGTT | 1051 |
| PRKCM | NM_002742 | CCUUCAUCACCCUGGUGUUTT | 1052 |
| PRKCM | NM_002742 | GUUCCCUGAAUGUGGUUUCTT | 1053 |
| PRKWNK3 | AJ409088 | ACCAAGCAGCCAGCUAUACTT | 1054 |
| PRKWNK3 | AJ409088 | CUAAUGACAUCUGGGACCUTT | 1055 |
| PRKWNK3 | AJ409088 | CUACGAAGGAAAACGUCAGTT | 1056 |
| PRKY | NM_002760 | AGACAGUGAAGCUGGUUGUTT | 1057 |
| PRKY | NM_002760 | GAAUUUCUGAGGACGAGCUTT | 1058 |
| PRKY | NM_002760 | UCAGAUUUGGGCCAGAGUUTT | 1059 |
| PTEN | NM_000314 | UGGAGGGGAAUGCUCAGAATT | 1060 |
| PTEN | NM_000314 | AAGGCAGCUAAAGGAAGUGTT | 1061 |
| PTEN | NM_000314 | UAAAGAUGGCACUUUCCCGTT | 1062 |
| PTK6 | NM_005975 | AACACCCUCUGCAAAGUUGTT | 1063 |
| PTK6 | NM_005975 | CCGUGGUUCUUUGGCUGCATT | 1064 |
| PTK6 | NM_005975 | UCAGGCUUAUCCGAUGUGCTT | 1065 |
| PTTG1 | NM_004219 | AACAGCCAAGCUUUUCUGCTT | 1066 |
| PTTG1 | NM_004219 | GGCUUUGGGAACUGUCAACTT | 1067 |
| PTTG1 | NM_004219 | UCUGUUGCAGUCUCCUUCATT | 1068 |
| RALA | NM_005402 | AGACAGGUUUCUGUAGAAGTT | 1069 |
| RALA | NM_005402 | GUCCAGAUCGAUAUCUUAGTT | 1070 |
| RALA | NM_005402 | GUUUAGCCAAGAGAAUCAGTT | 1071 |
| RALBP1 | NM_006788 | AAUGAAGAGGUCCAAGGGATT | 1072 |
| RALBP1 | NM_006788 | AGGACCCGUGCAUCUUACUTT | 1073 |
| RALBP1 | NM_006788 | GcUAAAAGAcAGGAGUGUGTT | 1074 |
| RAP1A | NM_002884 | CAGUGUAUGCUCGAAAUCCTT | 1075 |
| RAP1A | NM_002884 | GAUGAGCGAGUAGUUGGCATT | 1076 |
| RAP1A | NM_002884 | UUGGAAAGUGCCAGCAUUCTT | 1077 |
| RASA2 | NM_006506 | AACUGAUGACCUGGGGUCUTT | 1078 |
| RASA2 | NM_006506 | CAAGCAGAGAGCUCACCUATT | 1079 |
| RASA2 | NM_006506 | GAAAACAAGCAAUCCGCAGTT | 1080 |
| RET | NM_000323 | CUUCGCAGAAAAGAGUCGGTT | 1081 |
| RET | NM_000323 | GACAUCCAGGAUCCACUGUTT | 1082 |
| RET | NM_000323 | GUGUGCCGAACUUCACUACTT | 1083 |
| RHOK | NM_002929 | AGUACACAGCAGGUUCAUCTT | 1084 |
| RHOK | NM_002929 | CGUGAAUGAGGAGAACCCUTT | 1085 |
| RHOK | NM_002929 | GUUUAAGGAGGGGCCUGUGTT | 1086 |
| RPS6KA6 | NM_014496 | CCUCCUUUCAAACCUGCUUTT | 1087 |
| RPS6KA6 | NM_014496 | GAGGUUCUGUUUACAGAGGTT | 1088 |
| RPS6KA6 | NM_014496 | UCAGCCAGUGCAGAUUCAATT | 1089 |
| SGK2 | NM_016276 | AGAGCCUUAUGAUCGAGCATT | 1090 |
| SGK2 | NM_016276 | CUCUAUCAUGCCUGCUCCUTT | 1091 |
| SGK2 | NM_016276 | GAGAAGGACCUGUGAAACUTT | 1092 |
| SKP2 | NM_005983 | AAGAACCAGGAGAUAUGGGTT | 1093 |
| SKP2 | NM_005983 | GGUCUCUGGUGUUUGUAAGTT | 1094 |
| SKP2 | NM_005983 | UUUGCCCUGCAGACUUUGCTT | 1095 |
| SRC | NM_005417 | GAACCGGAUGCAGUUGAGCTT | 1096 |
| SRC | NM_005417 | GCCGGAAUACAAGAACGGGTT | 1097 |
| SRC | NM_005417 | GUGGCUCUUAUCCGCAUGATT | 1098 |
| SRPK1 | NM_003137 | CGCUUAUGGAACGUGAUACTT | 1099 |
| SRPK1 | NM_003137 | GCAACAGAAUGGCAGCGAUTT | 1100 |
| SRPK1 | NM_003137 | GUUCUAAUCGGAUCUGGCUTT | 1101 |
| STAT3 | NM_139276 | AUGCCACAGGCCACCUAUATT | 1102 |
| STAT3 | NM_139276 | CGACCUGCAGCAAUACCAUTT | 1103 |
| STAT3 | NM_139276 | GAAUCACAUGCCACUUUGGTT | 1104 |
| STAT4 | NM_003151 | ACACAGAUCUGCCUCUAUGTT | 1105 |
| STAT4 | NM_003151 | CCCUACAAUAAAGGCCGGUTT | 1106 |
| STAT4 | NM_003151 | UUAGGAAGGUCCUUCAGGGTT | 1107 |
| STAT5A | NM_003152 | CCUGUGGAACCUGAAACCATT | 1108 |
| STAT5A | NM_003152 | GUCUAUGAUGCUGUUGCCCTT | 1109 |
| STAT5A | NM_003152 | UGAGAUGAUUCAGAAGGGGTT | 1110 |
| STK4 | NM_006282 | CACCAUUUUGGAUGGCUCCTT | 1111 |
| STK4 | NM_006282 | GGAAAACCAGAUCAACAGCTT | 1112 |
| STK4 | NM_006282 | UUCUGGAUGGCUUGCCUCATT | 1113 |
| STK6 | NM_003600 | ACAGUCUUAGGAAUCGUGCTT | 1114 |
| STK6 | NM_003600 | GCACAAAAGCUUGUCUCCATT | 1115 |
| STK6 | NM_003600 | UUGCAGAUUUUGGGUGGUCTT | 1116 |
| TCF3 | M31523 | AAAGACCCCGUGUAAACCUTT | 1117 |
| TCF3 | M31523 | ACCUCAAGGCCAGCUCAAUTT | 1118 |
| TCF3 | M31523 | AUGGGGCAUUUUGUUGGGATT | 1119 |
| TERT | NM_003219 | CACCAAGAAGUUCAUCUCCTT | 1120 |
| TERT | NM_003219 | GAGUGUCUGGAGCAAGUUGTT | 1121 |
| TERT | NM_003219 | GUUUGGAAGAACCCCACAUTT | 1122 |
| TGFBR1 | NM_004612 | GACAUGAUUCAGCCACAGATT | 1123 |
| TGFBR1 | NM_004612 | UUCCUCGAGAUAGGCCGUUTT | 1124 |
| TGFBR1 | NM_004612 | UUUGGGAGGUCAGUUGUUCTT | 1125 |
| TK2 | NM_004614 | GAUGCCAGAAGUGGACUAUTT | 1126 |
| TK2 | NM_004614 | UACCUGGAAGCAAUUCACCTT | 1127 |
| TK2 | NM_004614 | UUAUGCUGCAUUUGGCUGGTT | 1128 |
| TOP2B | NM_001068 | ACAUUCCCUGGAGUGUACATT | 1129 |
| TOP2B | NM_001068 | GAGGAUUUAGCGGCAUUUGTT | 1130 |
| TOP2B | NM_001068 | GCUGCUGGACUGCAUAAAGTT | 1131 |
| TOP3A | NM_004618 | GAUCCUCCCUGUCUAUGAGTT | 1132 |
| TOP3A | NM_004618 | GCAAAGAAAUUGGACGAGGTT | 1133 |
| TOP3A | NM_004618 | GGCGAAAACAUCGGGUUUGTT | 1134 |
| TOP3B | NM_003935 | CAAAUGGGACAAAGUGGACTT | 1135 |
| TOP3B | NM_003935 | CUUUGACCUGAAGGGCUCUTT | 1136 |
| TOP3B | NM_003935 | UCCAGUCCUUCAAACCAGATT | 1137 |
| WASL | NM_003941 | AAACAGGAGGUGUUGAAGCTT | 1138 |
| WASL | NM_003941 | AAGUGGAGCAGAACAGUCGTT | 1139 |
| WASL | NM_003941 | GGACAAUCCACAGAGAUCUTT | 1140 |
| WEE1 | NM_003390 | AUCGGCUCUGGAGAAUUUGTT | 1141 |
| WEE1 | NM_003390 | CAAGGAUCUCCAGUCCACATT | 1142 |
| WEE1 | NM_003390 | UGUACCUGUGUGUCCAUCUTT | 1143 |
| WISP1 | NM_003882 | AAAUGCCUGUCUCUAGCUGTT | 1144 |
| WISP1 | NM_003882 | AUGGCCAGUUUUCUGGUAGTT | 1145 |
| WISP1 | NM_003882 | CCUGGGCAUUGUUGAGGUUTT | 1146 |
| WISP3 | NM_003880 | ACAGUUUUGUCACUGGCCCTT | 1147 |
| WISP3 | NM_003880 | CAAAAUGGACUCCCUGCUCTT | 1148 |
| WISP3 | NM_003880 | CCAGGGGAAAUCUGCAAUGTT | 1149 |
| WNT1 | NM_005430 | ACGGCGUUUAUCUUCGCUATT | 1150 |
| WNT1 | NM_005430 | CCCUCUUGCCAUCCUGAUGTT | 1151 |
| WNT1 | NM_005430 | CUAUUUAUUGUGCUGGGUCTT | 1152 |
| WNT2 | NM_003391 | AUUUGCCCGCGCAUUUGUGTT | 1153 |
| WNT2 | NM_003391 | AACGGGCGAUUAUCUCUGGTT | 1154 |
| WNT2 | NM_003391 | AGAAGAUGAAUGGUCUGGCTT | 1155 |
| WT1 | NM_024426 | CACUGGCACACUGCUCUUATT | 1156 |
| WT1 | NM_024426 | GACAAGAUACCGGUGCUUCTT | 1157 |
| WT1 | NM_024426 | GACACCAAAGGAGACAUACTT | 1158 |
| | NM_017719 | AGACCUAAUCACACGGAUGTT | 1159 |
| | NM_017719 | AGAUAGCGGGUUCACCUACTT | 1160 |
| | NM_017719 | GUUGACAGACUUUGGGUUCTT | 1161 |
| | XM_168069 | ACUCCAUCUGGUUGACCUGTT | 1162 |
| | XM_168069 | GAUUCAGGUGGAACUGAACTT | 1163 |
| | XM_168069 | GCACCAAGCUCCUCUGAUGTT | 1164 |
| | XM_170783 | ACCGACACUUUGGCUUCCATT | 1165 |
| | XM_170783 | GAUGAGCGCGGGAAUGUUGTT | 1166 |
| | XM_170783 | UGGCCGAGGCCUUCAAGCUTT | 1167 |
| | XM_064050 | CAUCAAUCACUCUCUGCUGTT | 1168 |
| | XM_064050 | CUAACCCAGGAUGUUCAGGTT | 1169 |
| | XM_064050 | GACACUCACCAUGCUGAAATT | 1170 |
| | XM_066649 | AAGGGUGACUUUGUGUCCUTT | 1171 |
| | XM_066649 | ACCAGGAACAAACCUGUUGTT | 1172 |
| | XM_066649 | UUUGAAGGUGGCCCUCCUATT | 1173 |
| | XM_089006 | AAAUCGAGAAGGAGGCUCATT | 1174 |
| | XM_089006 | AUAGUGACCGUCCCUUUGATT | 1175 |
| | XM_089006 | CCAGGUUCCUCCAAAGAUGTT | 1176 |
| | NM_145754 | AAGGGUUCAGCAUCUGACUTT | 1177 |
| | NM_145754 | CCUGGAGACAUUGCACCAGTT | 1178 |
| | NM_145754 | GGUGCUACCUCCUUUCCAGTT | 1179 |
| | NM_017596 | AGUUGCCCACCCUGUUUUUTT | 1180 |
| | NM_017596 | GAAAGAAUCCGUCCGCAUGTT | 1181 |
| | NM_017596 | GCAGCCAGAACUCUCAAAGTT | 1182 |
| | NM_139286 | GUUGUAGGAGGCAGUGAUGTT | 1183 |
| | NM_139286 | UCUCAAUUUGGAAGUCUUGTT | 1184 |
| | NM_139286 | UGAUCAAUGAUCGGAUUGGTT | 1185 |
| | NM_014885 | ACCAGGAUUUGGAGUGGAUTT | 1186 |
| | NM_014885 | CAAGGCAUCCGUUAUAUCUTT | 1187 |
| | NM_014885 | GUGGCUGGAUUCAUGUUCCTT | 1188 |
| | NM_016263 | CCAGAUCCUUGUCUGGAAGTT | 1189 |
| | NM_016263 | CGACAACAAGCUGCUGGUCTT | 1190 |
| | NM_016263 | GAAGCUGUCCAUGUUGGAGTT | 1191 |
| | NM_013367 | AGCCAGCAGAUGUAAUUGGTT | 1192 |
| | NM_013367 | CAUUUCAAUGAGGCUCCAGTT | 1193 |
| | NM_013367 | GUCAUUUACAGAGUGGCUCTT | 1194 |
| | NM_018492 | AGGACACUUUGGGUACCAGTT | 1195 |
| | NM_018492 | GACCCUAAAGAUCGUCCUUTT | 1196 |
| | NM_018492 | GCUGAGGAGAAUAUGCCUCTT | 1197 |
| | XM_168069 | CAAAGUUAUUAGCCCCAAGTT | 1198 |
| | XM_168069 | CAGAGGCCAAGUAUAUCAATT | 1199 |
| | XM_168069 | CCUGCAGAUUUGCACAGCGTT | 1200 |
| | NM_021170 | AUCCUGGAGAUGACCGUGATT | 1201 |
| | NM_021170 | GCCGGUCAUGGAGAAGCGGTT | 1202 |
| | NM_021170 | UGGCCCUGAGACUGCAUCGTT | 1203 |
| | NM_019089 | CCCCUCCAUGCUCAGAACUTT | 1204 |
| | NM_019089 | CCUAUCUGGGAAGCCUGUGTT | 1205 |
| | NM_019089 | UGCCCCAGUGACAAUAACATT | 1206 |
| | AK024504 | AGAGAGCUGGACCAUUCAUTT | 1207 |
| | AK024504 | AUGAGCAAUGCGGAUAGCUTT | 1208 |
| | AK024504 | GCCAUGUGUCUGAUGACAUTT | 1209 |
| | AB002301 | AGACAAAGAGGGGACCUUCTT | 1210 |
| | AB002301 | GAAAGUCUAUCCGAAGGCUTT | 1211 |
| | AB002301 | UGCCUCCCUGAAACUUCGATT | 1212 |
| | NM_018401 | AGGUAUGCAUCGUGCAGAATT | 1213 |
| | NM_018401 | GCAAUCAAACCGUCAUGACTT | 1214 |
| | NM_018401 | UAUCCUGCUGGAUGAACACTT | 1215 |
| | NM_016457 | CAUUGUCCACUGUGACUUGTT | 1216 |
| | NM_016457 | UGAAGUGGCCAUUCUGCAGTT | 1217 |
| | NM_016457 | UGUGGACAUUGCCACUGUCTT | 1218 |
| | NM_005200 | AUGAUCGCACCGCAGAGGUTT | 1219 |
| | NM_005200 | UACAUGACGUACUUGAGUGTT | 1220 |
| | NM_005200 | UGCUAAGGGGAUCGGACAUTT | 1221 |
| | NM_024322 | ACCACUCCGGAUACAUCACTT | 1222 |
| | NM_024322 | ACUAAGGCGUCUGCGAGAUTT | 1223 |
| | NM_024322 | GGACCUCACAGCAACUCUUTT | 1224 |
| | NM_017769 | CUGGUUGCAGUUCCAUUCCTT | 1225 |
| | NM_017769 | GUGAGCAUCCUGGAUCAAATT | 1226 |
| | NM_017769 | UUCAGAGAGUCCACACACCTT | 1227 |
| | NM_019013 | AAAACCCCCGGGAGUCGUCTT | 1228 |
| | NM_019013 | AGUGGCACCAAGUGGCUGGTT | 1229 |
| | NM_019013 | GAAACCUGCUUUGUCAUUUTT | 1230 |
| | AI338451 | CUGAUGCACUUUGCUGCAGTT | 1231 |
| | AI338451 | CUGCAGGUUCAAAUCCCAGTT | 1232 |
| | AI338451 | GGGGAAAAAGCUUUGCGUUTT | 1233 |
| | NM_018123 | UAUCGAGCCACCAUUUGUGTT | 1234 |
| | NM_018123 | UGAUGCAUAUAGCCGCAACTT | 1235 |
| | NM_018123 | UGCACAGGGCCAAAGUUGATT | 1236 |

### 6.4. Example 4

### BRCA1/BARD1 E3 Ubiquitin Ligase as an Anti-Cancer Drug Target

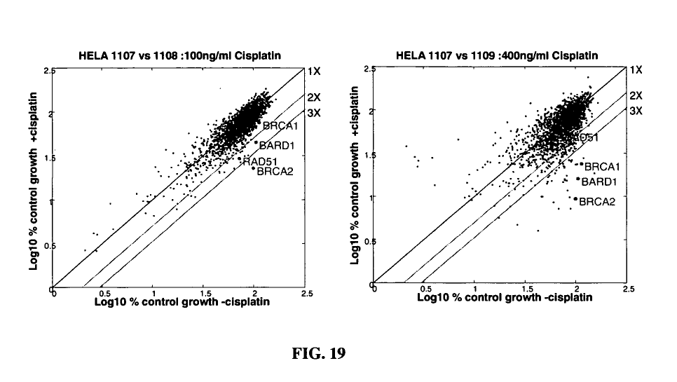

Examples 2 and 3 describe siRNA screens to identify genes that enhance cell killing by DNA damaging agents. In this example, HeLa cells were treated with or without cisplatin, and sensitization by a member of the BRCC complex were investigated (FIG. 19). Prominent amongst the genes whose disruption sensitized cells to DNA damage were BRCA1, BRCA2, BARD1 and RAD51, all members of the BRCC complex that enhances cellular survival following DNA damage (Dong et al., Mol Cell. 2003 November; 12 (5):1087-99). Sensitization by BRCA1, BRCA2 and BARD1 was dose dependent with respect to cisplatin concentration, but sensitization by RAD51 was only seen at low cisplatin concentration (FIG. 1). In other experiments, it was found that disruption of BRCA1 and BRCA2 decreased the IC50 concentrations for cisplatin inhibition of HeLa cell growth >˜4-fold (data not shown). Silencing by BRCA1, BRCA2 and BARD1 siRNA pools ranged from ˜85%-98% (data not shown). Table IV lists siRNA sequences of BARD1 and RAD51 used in this example.

These findings were remarkable in that products of the BRCA1, BRCA2, BARD21 and RAD51 genes are associated with a holoenzyme complex (BRCC) that enhances cellular survival following DNA damage (Dong et al., Mol Cell. 2003 November; 12 (5):1087-99). This complex has E3 Ub ligase activity, most of which can be recovered as a BRCA1/BARD1 heterodimer (Dong et al., Mol Cell. 2003 November; 12 (5):1087-99; Brzovic et al., Nat Struct Biol. 2001 October; 8 (10):833-7). These findings strongly implicate BRCC in mediating sensitivity to cisplatin in our siRNA screens. Surprisingly, siRNA pools to members of the FANC complex (FANCA, FANCC, FANCE and FANCF), another multisubunit complex implicated in determining resistance to cisplatin (Taniguchi et al., Nat Med. 2003 May; 9 (5):568-74), did not increase sensitivity in our assays (data not shown).

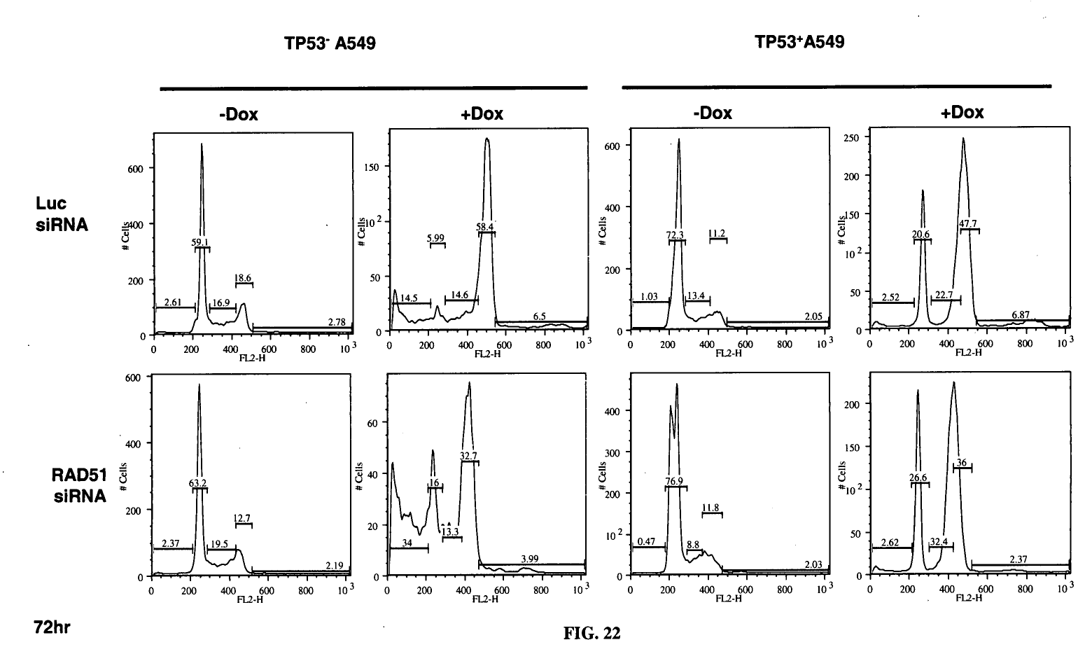

To determine if the sensitization to cisplatin by BRCA1 or BRCA2 disruption was affected by the presence or absence of TP53 expression in the target cells, matched pairs of TP53 positive and negative cells generated by stable expression of short hairpin RNAs (shRNAs) targeting TP53 (see, Example 2) were used. TP53-positive or negative cells were supertransfected with siRNA pools to BRCA1 or BRCA2, treated with cisplatin and analyzed for cell growth using Alamar Blue (FIG. 20). TP53-negative cells were ˜10-fold more sensitive to cisplatin when transfected with BRCA1 or BRCA2 siRNAs (IC50˜0.1 nM) than with control siRNA (luciferase, IC50-˜1 nM). The sensitization to cisplatin following BRCA1 or BRCA2 disruption was even more pronounced at lower cisplatin concentrations. TP53-positive cells were less sensitized to cisplatin following BRCA1 or BRCA2 disruption (IC50 ˜0.4 nM). Sensitization to cisplatin following BRCA1 or BRCA2 disruption was similar in magnitude in this assay to the sensitization seen following disruption of CHEK1 (data not shown). Sensitization to DNA damaging agents following BRCA1 and BRCA2 disruption was also investigated using cell cycle analysis. TP53-positive and negative cells were supertransfected with siRNA pools to BRCA1 or BRCA2, treated with one of several DNA damaging agents (cisplatin, camptothecin, doxorubicin and bleomycin) and analyzed for cell cycle distribution by flow cytometry. In all cases, TP53-negative cells were more sensitive to DNA damage following BRCA1 or BRCA2 disruption than in luciferase-transfected cells (data not shown). The response of these cells to bleomycin following BRCA1 disruption is shown in FIG. 21. BRCA1 disruption resulted in more sub-G1 cells (dead cells) following bleomycin treatment of TP53-negative than TP53-positive cells. We conclude that cells lacking TP53 are more sensitive to DNA damage following BRCA1 disruption. FIG. 22 shows results that demonstrate that RAD51/Doxorubicin synergy is greater in TP53− cells.

The cell lines used in this example were HeLa cells, TP53-positive A549 cells and TP53-negative A549 cells. The matched pair of TP53 positive and negative cells were generated by stable transfection of short hairpin RNAs (shRNAs) targeting TP53 (monthly highlt highlight, November 2003). The cells were transfected using pools of siRNAs (pool of 3 siRNA per gene) at 100 nM (each siRNA at 33 nM), or with single siRNA at 100 nM. The following siRNAs were used in our study: Luc control, BRCA1, BRCA2 and BARD1 pool. These transfected cells were then treated with varying concentrations of DNA damaging agents. The concentration for each agent used in the cell cycle analysis is as follows: for HeLa cells, Doxorubicin (10 nM), Camptothecin (6 nM), Cisplatin (400 ng/ml), Mitomycin C (40 nM), Bleomycin (100 ng/ml); for the other cell lines, Doxorubicin (200 nM), Camptothecin (200 nM), Cisplatin (2 ug/ml), Mitomycin C (400 nM), Bleomycin (5 ug/ml).

siRNA transfection was carried out as follows: one day prior to transfection, 2000 (or 100) microliters of a chosen cell line, e.g., cervical cancer HeLa cells (ATCC, Cat. No. CCL-2), grown in DMEM/10% fetal bovine serum (Invitrogen, Carlsbad, Calif.) to approximately 90% confluency were seeded in a 6-well (or 96-well) tissue culture plate at 45,000 (or 2000) cells/well. For each transfection 70 microliters of OptiMEM (Invitrogen) was mixed with 5 microliter of siRNA (Dharmacon, Lafayette, Colo.) from a 20 micromolar stock. For each transfection, a ratio of 20 microliter of OptiMEM was mixed with 5 microliter of Oligofectamine reagent (Invitrogen) and incubated 5 minutes at room temperature. Then 25-microliter OptiMEM/Oligofectamine mixture was mixed with the 75-microliter of OptiMEM/siRNA mixture, and incubated 15-20 minutes at room temperature. 100 (or 10) microliter of the transfection mixture was aliquoted into each well of the 6-well (or 96-well) plate and incubated for 4 hours at 37° C. and 5% CO₂.

After 4 hours, 100 microliter/well of DMEM/10% fetal bovine serum with or without DNA damage agents was added to each well to reach the final concentration of each agents as described above. The plates were incubated at 37° C. and 5% CO₂ for another 68 hours. Samples from the 6-well plates were analyzed for cell cycle profiles and samples from 96-well plates were analyzed for cell growth with Alamar Blue assay.

For cell cycle analysis, the supernatant from each well was combined with the cells that were harvested by trypsinization. The mixture was then centrifuged at 1200 rpm for 5 minutes. The cells were then fixed with ice cold 70% ethanol for ˜30 minutes. Fixed cells were washed once with PBS and resuspended in 0.5 ml of PBS containing Propidium Iodide (10 microgram/ml) and RNase A(1 mg/ml), and incubated at 37° C. for 30 min. Flow cytometric analysis was carried out using a FACSCalibur flow cytometer (Becton Dickinson) and the data was analyzed using FlowJo software (Tree Star, Inc). The Sub-G1 cell population was used to measure cell death. If the summation of the Sub-G1 population from the (siRNA+DMSO) sample and (Luc+drug) sample is larger than the Sub-G1 population of (siRNA+drug) sample, we define that as sensitization of siRNA silencing to DNA damage.

For Alamar Blue assay, the media from the 96-well plates was removed, and 100 uL/well complete media containing 10% (vol/vol) alamarBlue reagent (BioSource International, Inc) and 1/100^{th} volume 1M Hepes buffer tissue culture reagent was added. The plates were then incubated 1-4 hours at 37° C. and fluorescence was measured by exciting at 544 nm and detecting emission at 590 nm with SPECTRAMax Gemini-Xs Spectrofluorometer (Molceular Devices). The fluorescence signal was corrected for background (no cells). Cell response (survival) in the presence of DNA damaging agents was measured as a percentage of control cell growth in the absence of DNA damaging agents.

Many functions have been ascribed to BRCA1, but the only know enzymatic function is E3 Ub ligase activity. This activity is enhanced by association of BARD1 with BRCA1 and results in autoubiquitylation of the BRCA1/BARD1 complex via an unconventional K6 linkage of ubiquitin (Wu-Baer et al., J Biol. Chem. 2003 Sep. 12; 278 (37):34743-6; Chen et al., J Biol. Chem. 2002 Jun. 14; 277 (24):22085-92), Available evidence suggests that the BRCA1 E3 Ub ligase activity is required for its DNA repair function. Cancer-predisposing mutations within the BRCA1 RING domain abolish its Ub ligase activity and these mutants are unable to reverse gamma-radiation hypersensitivity of BRCA1-null human breast cancer cells (Ruffner et al., Proc Natl Acad Sci USA. 2001 Apr. 24; 98 (9):5134-9). In addition, siRNA-mediated disruption of BRCA1 blocks deposition of polyubiquitin structures in nuclear foci that are sites of DNA repair and checkpoint activation in gamma-irradiated cells (Morris et al., Hum Mol Genet. 2004 Apr. 15; 13 (8):807-17). It is important to note that the ubiquitin linkage (K6) mediated by BRCA1 is distinct from the ubiquitin linkage (K48) that marks proteins for degradation by the proteasome (Wu-Baer et al., J Biol. Chem. 2003 Sep. 12; 278 (37):34743-6; Morris et al., Hum Mol Genet. 2004 Apr. 15; 13 (8):807-17). The function of the K6 linkage is currently unknown, but may serve a signaling function.

Taken together, these findings and those in the literature suggest that an inhibitor of BRCA1 E3 Ub ligase activity might be an effective anti-cancer agent because it would enhance the therapeutic window for DNA damaging agents towards tumor cells (most of which are TP53-negative) relative to normal cells (TP53-positive). Dose-dependence of BRCA1 levels on enhanced sensitivity to cisplatin versus deposition of polyubiquitin in nuclear foci is carried out to gain insight into whether these events are causally linked. Chemical inhibitors of BRCA1 E3 Ub ligase activity are also investigated to establish the role of ubiquitylation in repair of DNA damage.

Evidence suggesting the existence of other E3 Ub ligases with roles in DNA damage repair comes from studies in yeast (Spence et al., Mol Cell Biol. 1995 March; 15 (3):1265-73) showing that DNA damage repair requires Ub ligases with non-proteolytic specificity (K63 linkage). To expedite the identification of those involved in DNA damage repair, we are adding siRNAs for multiple E3 ligases with similar domain structures to BRCA1 (RING finger domain ligases) to our siRNA library with the expectation that those that sensitize cells to DNA damage will be revealed by our library screens.

Table IV siRNA sequences of BARD1 and RAD51

| | | | | |
|---|---|---|---|---|
| | | | | SEQ |
| CONTENTS | | SEQUENCE | GENE | ID |
| ID | SENSE SEQ | ID | NAME | NO |

| | | | | |
|---|---|---|---|---|
| 5093 | CAGUAAUUCUUAAGGCUAATT | NM_000465 | BARD1 | 1237 |
| 5094 | CUCCUGAGAAGGUCUGCAATT | NM_000465 | BARD1 | 1238 |
| 5095 | CGCAGAAGCAGGCUCAACATT | NM_000465 | BARD1 | 1239 |
| 6920 | GUUAGAGCAGUGUGGCAUATT | NM_002875 | RAD51 | 1240 |
| 6921 | GGUAUGCACUGCUUAUUGUTT | NM_002875 | RAD51 | 1241 |
| 6922 | CAGAUUGUAUCUGAGGAAATT | NM_002875 | RAD51 | 1242 |

### 7. REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of the present invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

1. A method for identifying a gene whose product modulates the effect of an agent on a cell of a cell type, said method comprising
(a) contacting a plurality of groups of one or more cells of said cell type with said agent, wherein each said group of one or more cells comprises one or more different small interfering RNAs (siRNAs) from among a plurality of different siRNAs, said one or more different siRNAs targeting a same gene, and said plurality of different siRNAs comprising siRNAs targeting respectively different genes in cells of said cell type;
(b) comparing the effect of said agent on each said group of one or more cells to the effect of said agent on a cell of said cell type which does not comprise an siRNA targeting any one of said different genes; and
(c) identifying a gene as said gene whose product modulates the effect of said agent on a cell of said cell type if the effect of said agent on said group of one or more cells comprising said one or more different siRNAs targeting said gene is different as compared to the effect of said agent on a cell of said cell type which does not comprise an siRNA targeting any one of said different genes.

2. The method of claim 1, wherein each said group of cells comprising one or more of said plurality of siRNAs is obtained by transfection with said one or more siRNAs prior to said step of contacting.

3. A method for identifying a gene whose product modulates the effect of an agent on a cell of a cell type, said method comprising
(a) transfacting each of a plurality of groups of one or more cells of said cell type with a composition comprising one or more different small interfering RNAs (siRNAs) from among a plurality of different siRNAs, said one or more different siRNAs targeting a same gene, and said plurality of different siRNAs comprising siRNAs targeting respectively different genes in cells of said cell type;
(b) contacting each of said plurality of groups of one or more cells with said agent;
(c) comparing the effect of said agent on each said group of one or more cells to the effect of said agent on a cell of said cell type which is not transfected with an siRNA targeting any one of said different genes; and
(d) identifying a gene as said gene whose product modulates the effect of said agent on a cell of said cell type if the effect of said agent on said group of one or more cells comprising said one or more different siRNAs targeting said gene is different as compared to the effect of said agent on a cell of said cell type which does not comprise an siRNA targeting any one of said different genes.

4. The method of any one of claims 1-3, wherein the effect of said agent on said group of one or more cells comprising said siRNA is enhanced as compared to the effect of said agent on a cell of said cell type which does not comprise an siRNA targeting any one of said different genes.

5. The method of any one of claims 1-3, wherein the effect of said agent on said group of one or more cells comprising said siRNA is reduced as compared to the effect of said agent on a cell of said cell type which does not comprise an siRNA targeting any one of said different genes.

6. The method of any one of claims 1-3, wherein said agent acts on a gene other than any one of said different genes targeted by said plurality of siRNAs, or a protein encoded thereof.

7. The method of claim 3, wherein said plurality of siRNAs comprises at least k different siRNAs targeting at least one gene of said different genes, wherein said k is selected from the group consisting of 2, 3, 4, 5, 6 and 10.

8. The method of claim 7, wherein said one or more different siRNAs targeting said at least one gene comprise 2, 3, 4, 5, 6, or 10 different siRNAs.

9. The method of claim 7, wherein said plurality of siRNAs comprises at least k different siRNAs targeting each of at least 2 different genes of said different genes, wherein said k is selected from the group consisting of 2, 3, 4, 5, 6 and 10.

10. The method of claim 9, wherein said one or more different siRNAs targeting each said at least 2 different genes comprise 2, 3, 4, 5, 6, or 10 different siRNAs.

11. The method of claim 9, wherein said plurality of siRNAs comprises at least k different siRNAs targeting each of said different genes, wherein said k is selected from the group consisting of 2, 3, 4, 5, 6 and 10.

12. The method of claim 11, wherein said one or more different siRNAs targeting each of said different genes comprise 2, 3, 4, 5, 6, or 10 different siRNAs.

13. The method of claim 5, wherein said cell type is a cancer cell type.

14. The method of claim 13, wherein said cell type is a cancer cell type, and wherein said effect is growth inhibitory effect.

15. The method of claim 12, wherein said agent is a KSP inhibitor.

16. The method of any one of claims 7-15, wherein said plurality of different genes comprises at least N different genes, wherein N is selected from the group consisting of 5, 10, 100, 1,000, and 5,000 different genes.

17. The method of any one of claims 1-3, wherein said different genes are different endogenous genes.

18. A method for identifying a gene which interacts with a primary target gene in a cell of a cell type, said method comprising
(a) contacting a plurality of groups of one or more cells of said cell type with an agent, wherein said agent modulates the expression of said primary target gene and/or the activity of a protein encoded by said primary target gene, and wherein each said group of cells comprises one or more different siRNAs among a plurality of different siRNAs, said one or more different siRNAs targeting a same gene, and said plurality of different siRNAs comprising siRNAs targeting respectively different secondary genes in said cell;
(b) comparing the effect of said agent on each said group of one or more cells to the effect of said agent on a cell of said cell type which does not comprise an siRNA targeting any one of said different secondary genes; and
(c) identifying a gene as said gene that interacts with said primary target gene in a cell of said cell type if the effect of said agent on said group of one or more cells comprising one or more siRNAs targeting said gene is different as compared to the effect of said agent on a cell of said cell type which does not comprise an siRNA targeting any one of said different secondary genes.

19. The method of claim 18, wherein each said group of cells comprising one or more of said plurality of siRNAs is obtained by transfection with said one or more siRNA prior to said step of contacting.

20. A method for identifying a gene which interacts with a primary target gene in a cell of a cell type, said method comprising
(a) transfacting each of a plurality of groups of one or more cells of said cell type with a composition comprising one or more different small interfering RNAs (siRNAs) from among a plurality of different siRNAs, said one or more different siRNAs targeting a same gene, and said plurality of different siRNAs comprising siRNAs targeting respectively different genes in cells of said cell type;
(b) contacting said plurality of groups of one or more cells of said cell type with an agent, wherein said agent modulates the expression of said primary target gene and/or the activity of a protein encoded by said primary target gene;
(c) comparing the effect of said agent on each said group of one or more cells to the effect of said agent on a cell of said cell type which does not comprise an siRNA targeting any one of said different secondary genes; and
(d) identifying a gene as said gene that interacts with said primary target gene in a cell of said cell type if the effect of said agent on said group of one or more cells comprising one or more siRNAs targeting said gene is different as compared to the effect of said agent on a cell of said cell type which does not comprise an siRNA targeting any one of said different secondary genes.

21. The method of any one of claims 18-20, wherein said agent is an siRNA targeting and silencing said primary target gene.

22. The method of any one of claims 18-20, wherein said agent is an inhibitor of said primary target gene.

23. The method of any one of claims 18-20, wherein the effect of said agent on said group of one or more cells comprising said one or more siRNAs is enhanced as compared to the effect of said agent on a cell of said cell type which does not comprise an siRNA targeting any one of said different secondary genes.

24. The method of any one of claims 18-20, wherein the effect of said agent on said group of one or more cell comprising said one or more siRNAs is reduced as compared to the effect of said agent on a cell of said cell type which does not comprise an siRNA targeting any one of said different secondary genes.

25. The method of claim 20, wherein said plurality of siRNAs comprises at least k different siRNAs targeting at least one of said different secondary genes, wherein said k is selected from the group consisting of 2, 3, 4, 5, 6 and 10.

26. The method of claim 25, wherein said one or more different siRNAs targeting said at least one gene comprise 2, 3, 4, 5, 6, or 10 different siRNAs.

27. The method of claim 18, wherein said plurality of siRNAs comprises at least k different siRNAs targeting each of at least 2 different genes of said different secondary genes, wherein said k is selected from the group consisting of 2, 3, 4, 5, 6 and 10.

28. The method of claim 27, wherein said one or more different siRNAs targeting each said at least 2 different genes comprise 2, 3, 4, 5, 6, or 10 different siRNAs.

29. The method of claim 27, wherein said plurality of siRNAs comprises at least k different siRNAs targeting each of said different secondary genes, wherein said k is selected from the group consisting of 2, 3, 4, 5, 6 and 10.

30. The method of claim 29, wherein said one or more different siRNAs targeting each of said different genes comprise 2, 3, 4, 5, 6, or 10 different siRNAs.

31. The method of claim 22, wherein said primary target gene is KSP.

32. The method of claim 18, wherein said plurality of different genes comprises at least N different genes, wherein N is selected from the group consisting of 5, 10, 100, 1,000, and 5,000 different genes.

33. The method of any one of claims 18-20, wherein said different secondary genes are different endogenous genes.

34. The method of any one of claims 18-20, wherein said cell type is a cancer cell type.

35. The method of claim 8 or 26, wherein the total siRNA concentration of said one or more siRNAs in said composition is an optimal concentration for silencing said target gene, wherein said optimal concentration is a concentration further increase of which does not increase the level of silencing substantially.

36. The method of claim 35, wherein said optimal concentration is a concentration further increase of which does not increase the level of silencing by more than 20%, more than 10%, or more than 5%.

37. The method of claim 35, wherein the concentration of each said one or more siRNA is about the same.

38. The method of claim 35, wherein the respective concentrations of said one or more siRNAs are different from each other by less than 50%, less than 20%, or less than 10%.

39. The method of claim 35, wherein none of the siRNAs in said composition has a concentration that is more than 80%, more than 50%, or more than 20% of said total siRNA concentration of said one or more siRNAs.

40. The method of claim 35, wherein at least one siRNA in said composition has a concentration that is more than 20% or more than 50% of said total siRNA concentration of said one or more siRNAs.

41. The method of claim 8 or 26, wherein the number of different siRNAs and the concentration of each siRNA in said composition is chosen such that said composition causes less than 10%, less than 1%, less than 0.1%, or less than 0.01% of silencing of any off-target genes.

42. A method for treating a mammal having a cancer, comprising administering to said mammal a therapeutically sufficient amount of an agent, said agent regulating the expression of a STK6 or TPX2 gene and/or activity of a protein encoded by said STK6 or TPX2 gene, wherein said mammal is subject to a therapy comprising administering to said mammal a therapeutically sufficient amount of a KSP inhibitor.

43. A method for treating a mammal having a cancer, comprising administering to said mammal i) a therapeutically sufficient amount of an agent, said agent regulating the expression of a STK6 or TPX2 gene and/or activity of a protein encoded by said STK6 or TPX2 gene, and ii) a therapeutically sufficient amount of a KSP inhibitor.

44. The method of claim 42 or 43, wherein said agent reduces the expression of said STK6 or TPX2 gene in cells of said cancer.

45. The method of claim 42 or 43, wherein said agent comprises an siRNA targeting said STK6 or TPX2 gene.

46. The method of claim 45, wherein said agent comprises 2, 3, 4, 5, 6, or 10 different siRNAs targeting said STK6 or TPX2 gene.

47. The method of claim 46, wherein the total siRNA concentration of said different siRNAs in said agent is an optimal concentration for silencing said STK6 or TPX2 gene, wherein said optimal concentration is a concentration further increase of which does not increase the level of silencing substantially.

48. The method of claim 47, wherein said optimal concentration is a concentration further increase of which does not increase the level of silencing by more than 20%, more than 10%, or more than 5%.

49. The method of claim 47, wherein the concentration of each said different siRNA is about the same.

50. The method of claim 47, wherein the respective concentrations of said different siRNAs are different from each other by less than 50%, less than 20%, or less than 10%.

51. The method of claim 47, wherein none of the siRNAs in said agent has a concentration that is more than 80%, more than 50%, or more than 20% of said total siRNA concentration of said different siRNAs.

52. The method of claim 47, wherein at least one siRNA in said agent has a concentration that is more than 20% or more than 50% of said total siRNA concentration of said different siRNAs.

53. The method of claim 47, wherein the number of different siRNAs and the concentration of each siRNA in said agent is chosen such that said agent causes less than 10%, less than 1%, less than 0.1%, or less than 0.01% of silencing of any off-target genes.

54. The method of claim 45, wherein said mammal is a human, and wherein said siRNA is selected from the group consisting of siRNAs described by SEQ ID NO:1, SEQ ID NO:2, SEQ ID NO:3, SEQ ID NO:4, SEQ ID NO:5, and SEQ ID NO:6.

55. A method for treating a mammal having a cancer, comprising administering to said mammal i) a therapeutically sufficient amount of a first agent, said first agent regulating the expression of a STK6 or TPX2 gene and/or activity of a protein encoded by said STK6 or TPX2 gene, and ii) a therapeutically sufficient amount of a second agent, said second agent regulating the expression of a KSP gene and/or activity of a protein encoded by said KSP gene.

56. The method of claim 55, wherein said first agent comprises an siRNA targeting said STK6 or TPX2 gene, and said second agent comprises an siRNA targeting said KSP gene.

57. The method of claim 56, wherein said first agent comprises 2, 3, 4, 5, 6, or 10 different siRNAs targeting said STK6 or TPX2 gene.

58. The method of claim 57, wherein the total siRNA concentration of said different siRNAs in said first agent is an optimal concentration for silencing said STK6 or TPX2 gene, wherein said optimal concentration is a concentration further increase of which does not increase the level of silencing substantially.

59. The method of claim 58, wherein said optimal concentration is a concentration further increase of which does not increase the level of silencing by more than 20%, more than 10%, or more than 5%.

60. The method of claim 58, wherein the concentration of each said different siRNA is about the same.

61. The method of claim 58, wherein the respective concentrations of said different siRNAs are different from each other by less than 50%, less than 20%, or less than 10%.

62. The method of claim 58, wherein none of the siRNAs in said first agent has a concentration that is more than 80%, more than 50%, or more than 20% of said total siRNA concentration of said different siRNAs.

63. The method of claim 58, wherein at least one siRNA in said first agent has a concentration that is more than 20% or more than 50% of said total siRNA concentration of said different siRNAs.

64. The method of claim 58, wherein the number of different siRNAs and the concentration of each siRNA in said first agent is chosen such that said first agent causes less than 10%, less than 1%, less than 0.1%, or less than 0.01% of silencing of any off-target genes.

65. The method of claim 56, wherein said mammal is a human, and wherein said siRNA targeting said STK6 gene is selected from the group consisting of siRNAs described by SEQ ID NO:1, SEQ ID NO:2, SEQ ID NO:3, SEQ ID NO:4, SEQ ID NO:5, and SEQ ID NO:6.

66. The method of claim 45 or 56, wherein said mammal is a human, and wherein said siRNA targeting said TPX2 gene is selected from the group consisting of siRNAs described by SEQ ID NO:1237, SEQ ID NO:1238, and SEQ ID NO:1239.

67. A method for evaluating resistance of a cell to the growth inhibitory effect of a KSP inhibitor, said method comprising determining an expression level of a STK6 or TPX2 gene in said cell, wherein said expression level above a predetermined threshold level indicates that said cell is resistant to the growth inhibitory effect of said KSP inhibitor.

68. The method of claim 67, wherein said expression level of said STK6 or TPX2 gene is determined by a method comprising measuring the expression level of said STK6 or TPX2 gene using one or more polynucleotide probes, each of said one or more polynucleotide probes comprising a nucleotide sequence in said STK6 or TPX2 gene.

69. The method of claim 67 or 68, wherein said one or more polynucleotide probes are polynucleotide probes on a microarray.

70. A method for evaluating resistance of a cell to the growth inhibitory effect of a KSP inhibitor, said method comprising determining a level of abundance of a protein encoded by a STK6 or TPX2 gene in said cell, wherein said level of abundance of said protein above a predetermined threshold level indicates that said cell is resistant to the growth inhibitory effect of said KSP inhibitor.

71. A method for evaluating resistance of a cell to the growth inhibitory effect of a KSP inhibitor, said method comprising determining a level of activity of a protein encoded by a STK6 or TPX2 gene in said cell, wherein said activity level above a predetermined threshold level indicates that said cell is resistant to the growth inhibitory effect of said KSP inhibitor.

72. The method of claim 70 or 71, wherein said cell is a human cell.

73. A method for regulating resistance of a cell to the growth inhibitory effect of a KSP inhibitor, comprising contacting said cell with a sufficient amount of an agent, said agent regulating the expression of a STK6 or TPX2 gene and/or the activity of a protein encoded by said STK6 or TPX2 gene.

74. A method for regulating resistance of a cell to the growth inhibitory effect of a KSP inhibitor in a mammal, comprising administering to said mammal a therapeutically sufficient amount of an agent, said agent regulating the expression of a STK6 or TPX2 gene and/or the activity of a protein encoded by said STK6 or TPX2 gene.

75. A method for regulating growth of a cell, comprising contacting said cell with i) a sufficient amount of an agent that regulates the expression of a STK6 or TPX2 gene and/or the activity of a protein encoded by said STK6 or TPX2 gene; and ii) a sufficient amount of a KSP inhibitor.

76. The method of claim 73, 74, or 75, wherein said agent reduces the expression of said STK6 or TPX2 gene in said cell.

77. The method of claim 73, 74, or 75, wherein said agent comprises an siRNA targeting said STK 6 gene.

78. The method of claim 77, wherein said agent comprises 2, 3, 4, 5, 6, or 10 different siRNAs targeting said STK6 or TPX2 gene.

79. The method of claim 78, wherein the total siRNA concentration of said different siRNAs in said agent is an optimal concentration for silencing said STK6 or TPX2 gene, wherein said optimal concentration is a concentration further increase of which does not increase the level of silencing substantially.

80. The method of claim 79, wherein said optimal concentration is a concentration further increase of which does not increase the level of silencing by more than 20%, more than 10%, or more than 5%.

81. The method of claim 79, wherein the concentration of each said different siRNA is about the same.

82. The method of claim 79, wherein the respective concentrations of said different siRNAs are different from each other by less than 50%, less than 20%, or less than 10%.

83. The method of claim 79, wherein none of the siRNAs in said agent has a concentration that is more than 80%, more than 50%, or more than 20% of said total siRNA concentration of said different siRNAs.

84. The method of claim 79, wherein at least one siRNA in said agent has a concentration that is more than 20% or more than 50% of said total siRNA concentration of said different siRNAs.

85. The method of claim 79, wherein the number of different siRNAs and the concentration of each siRNA in said agent is chosen such that said agent causes less than 10%, less than 1%, less than 0.1%, less than 0.01% of silencing of any off-target genes.

86. The method of claim 77, wherein said cell is a human cell, and wherein said siRNA is selected from the group consisting of siRNAs described by SEQ ID NO:1, SEQ ID NO:2, SEQ ID NO:3, SEQ ID NO:4, SEQ ID NO:5, and SEQ ID NO:6.

87. The method of claim 77, wherein said cell is a human cell, and wherein said siRNA is selected from the group consisting of siRNAs described by SEQ ID NO:1237, SEQ ID NO:1238, and SEQ ID NO:1239.

88. A method of identifying an agent that is capable of regulating resistance of a cell to the growth inhibitory effect of a KSP inhibitor, wherein said agent is capable of modulating the expression of a STK6 or TPX2 gene and/or the activity of a protein encoded by said STK6 or TPX2 gene, said method comprising comparing inhibitory effect of said KSP inhibitor on cells expressing said STK6 or TPX2 gene in the presence of said agent with inhibitory effect of said KSP inhibitor on cells expressing said STK6 or TPX2 gene in the absence of said agent, wherein a difference in said inhibitory effect of said KSP inhibitor identifies said agent as capable of regulating resistance of said cell to the growth inhibitory effect of said KSP inhibitor.

89. A method of identifying an agent that is capable of regulating resistance of a cell to the growth inhibitory effect of a KSP inhibitor, wherein said agent is capable of modulating the expression of a STK6 or TPX2 gene and/or activity of a protein encoded by said STK6 or TPX2 gene, said method comprising:
(a) contacting a first cell expressing said STK6 or TPX2 gene with said KSP inhibitor in the presence of said agent and measuring a first growth inhibitory effect;
(b) contacting a second cell expressing said STK6 or TPX2 gene with said KSP inhibitor in the absence of said agent and measuring a second growth inhibitory effect; and
(c) comparing said first and second inhibitory effects measured in said step (a) and (b),
wherein a difference between said first and second inhibitory effects identifies said agent as capable of regulating resistance of a cell to the growth inhibitory effect of said KSP inhibitor.

90. The method of claim 88 or 89, wherein said agent comprises a molecule which reduces expression of said STK6 or TPX2 gene.

91. The method of claim 88 or 89, wherein said agent is an siRNA targeting said STK6 or TPX2 gene.

92. The method of claim 91, wherein said cell is a human cell, and wherein said siRNA is selected from the group consisting of siRNAs described by SEQ ID NO:1, SEQ ID NO:2, SEQ ID NO:3, SEQ ID NO:4, SEQ ID NO:5, and SEQ ID NO:6.

93. The method of claim 91, wherein said cell is a human cell, and wherein said siRNA is selected from the group consisting of siRNAs described by SEQ ID NO:1237, SEQ ID NO:1238, and SEQ ID NO:1239.

94. A cell comprising one or more different small interfering RNAs (siRNAs) targeting a STK6 or TPX2 gene in said cell.

95. The cell of claim 94, wherein said one or more different siRNAs comprises 2, 3, 4, 5, 6, or 10 different siRNAs.

96. The cell of claim 95, wherein said cell is produced by transfection using a composition of said one or more different siRNAs, wherein the total siRNA concentration of said composition is an optimal concentration for silencing said STK6 or TPX2 gene, wherein said optimal concentration is a concentration further increase of which does not increase the level of silencing substantially.

97. The cell of claim 96, wherein said optimal concentration is a concentration further increase of which does not increase the level of silencing by more than 20%, more than 10%, or more than 5%.

98. The cell of claim 96, wherein the concentration of each said different siRNA is about the same.

99. The cell of claim 96, wherein the respective concentrations of said different siRNAs are different from each other by less than 50%, less than 20%, or less than 10%.

100. The cell of claim 96, wherein none of the siRNAs in said composition has a concentration that is more than 80%, more than 50%, or more than 20% of said total siRNA concentration of said different siRNAs.

101. The cell of claim 96, wherein at least one siRNA in said composition has a concentration that is more than 20% or more than 50% of said total siRNA concentration of said different siRNAs.

102. The cell of claim 96, wherein the number of different siRNAs and the concentration of each siRNA in said composition is chosen such that said composition causes less than 10%, less than 1%, less than 0.1%, or less than 0.01% of silencing of any off-target genes.

103. The cell of claim 94, wherein said cell is a human cell.

104. The cell of claim 103, wherein said cell is a human cell, and wherein each of said one or more different siRNAs is selected from the group consisting of siRNAs described by SEQ ID NO:1, SEQ ID NO:2, SEQ ID NO:3, SEQ ID NO:4, SEQ ID NO:5, and SEQ ID NO:6.

105. The cell of claim 103, wherein said cell is a human cell, and wherein said siRNA is selected from the group consisting of siRNAs described by SEQ ID NO:1237, SEQ ID NO:1238, and SEQ ID NO:1239.

106. The cell of claim 94, wherein said cell is a murine cell.

107. A microarray for diagnosing resistance of a cell to the growth inhibitory effect of a KSP inhibitor, said microarray comprising one or more polynucleotide probes, wherein each said polynucleotide probe comprises a nucleotide sequence in a STK6 or TPX2 gene.

108. A kit for diagnosis of resistance of a cell to the growth inhibitory effect of a KSP inhibitor, comprising in one or more containers one or more polynucleotide probes, wherein each said polynucleotide probe comprises a nucleotide sequence in a STK6 or TPX2 gene.

109. A kit for screening for agents which regulate resistance of a cell to the growth inhibitory effect of a KSP inhibitor, comprising in one or more containers (i) the cell of claim 94; and (ii) a KSP inhibitor.

110. A kit for treating a mammal having a cancer, comprising in one or more containers (i) a sufficient amount of an agent that regulates the expression of a STK6 or TPX2 gene and/or the activity of a protein encoded by said STK6 or TPX2 gene; and (ii) a KSP inhibitor.

111. The method of any one of claims 42-43, 67, 70-71, 74-75 and 88-89, wherein said KSP inhibitor is (1S)-1-{[(2S)-4-(2,5-difluorophenyl)-2-phenyl-2,5-dihydro-1H-pyrrol-1-yl]carbonyl}-2-methylpropylamine.

112. The method of claim 1, 2, or 3, wherein said contacting step (a) is carried out separately for each said group of one or more cells.

113. The method of claim 18, 19, or 20, wherein said contacting step (a) is carried out separately for each said group of one or more cells.

114. The kit of claim 109 or 110, wherein said KSP inhibitor is (1S)-1-{[(2S)-4-(2,5-difluorophenyl)-2-phenyl-2,5-dihydro-1H-pyrrol-1-yl]carbonyl}-2-methylpropylamine.

115. A method for identifying a gene that interacts with a primary target gene in a cell of a cell type, said method comprising
(a) contacting one or more cells of said cell type with an agent, wherein said agent modulates the expression of a secondary target gene and/or the activity of a protein encoded by said secondary target gene, and wherein said one or more cells express a first small interference RNA (siRNA) targeting said primary target gene;
(b) comparing the effect of said agent on said one or more cells of said clone to the effect of said agent on a cell of said cell type not expressing said first siRNA; and
(c) identifying said secondary target gene as a gene that interacts with said primary target gene in a cell of said cell type if the effect of said agent on said one or more cells expressing said first siRNA is different as compared to the effect of said agent on a cell of said cell type not expressing said first siRNA.

116. A method for identifying a gene which interacts with a primary target gene in a cell of a cell type, said method comprising
(a) generating a clone of cells of said cell type which express a first small interference RNA (siRNA) targeting said primary target gene;
(b) contacting one or more cells of said clone with an agent, wherein said agent modulates the expression of a secondary target gene and/or the activity of a protein encoded by said secondary target gene;
(c) comparing the effect of said agent on said one or more cells of said clone to the effect of said agent on a cell of said cell type not expressing said first siRNA; and
(d) identifying said secondary target gene as a gene that interacts with said primary target gene in a cell of said cell type if the effect of said agent on said one or more cells expressing said first siRNA is different as compared to the effect of said agent on a cell of said cell type not expressing said first siRNA.

117. The method of claim 116, wherein said first siRNA is expressed by a nucleotide sequence integrated in the genome of said cells.

118. The method of claim 116, wherein said agent comprises one or more second siRNAs targeting and silencing said secondary target gene.

119. The method of claim 116, wherein said agent is an inhibitor of said secondary target gene.

120. The method of claim 118, wherein the effect of said agent on said one or more cells expressing said first siRNA is enhanced as compared to the effect of said agent on a cell of said cell type not expressing said first siRNA.

121. The method of claim 118, wherein the effect of said agent on said one or more cells expressing said first siRNA is reduced as compared to the effect of said agent on a cell of said cell type not expressing said first siRNA.

122. The method of claim 120, wherein said one or more second siRNAs comprises at least k different siRNAs, wherein said k is selected from the group consisting of 2, 3, 4, 5, 6 and 10.

123. The method of claim 122, wherein the total siRNA concentration of said at least k different siRNAs in said agent is an optimal concentration for silencing said secondary target gene, wherein said optimal concentration is a concentration further increase of which does not increase the level of silencing substantially.

124. The method of claim 123, wherein said optimal concentration is a concentration further increase of which does not increase the level of silencing by more than 20%, more than 10%, or more than 5%.

125. The method of claim 123, wherein the concentration of each said at least k different siRNA is about the same.

126. The method of claim 123, wherein the respective concentrations of said at least k different siRNAs are different from each other by less than 50%, less than 20%, or less than 10%.

127. The method of claim 123, wherein none of the siRNAs in said agent has a concentration that is more than 80%, more than 50%, or more than 20% of said total siRNA concentration of said different siRNAs.

128. The method of claim 123, wherein at least one siRNA in said agent has a concentration that is more than 20% or more than 50% of said total siRNA concentration of said at least k different siRNAs.

129. The method of claim 123, wherein the number of different siRNAs and the concentration of each siRNA in said agent is chosen such that said agent causes less than 10%, less than 1%, less than 0.1%, or less than 0.01% of silencing of any off-target genes.

130. The method of claim 122, wherein said cell type is a cancer cell type, and wherein said primary target gene is p53.

131. The method of claim 130, further comprising a step (e) repeating steps (b)-(d) for each of a plurality of different secondary target genes.

132. The method of claim 131, wherein said plurality of secondary target genes comprises at least the number of different genes selected from the group consisting of 5, 10, 100, 1,000, and 5,000 different genes.

133. The method of claim 132, wherein said effect is a change in the sensitivity of cells of said cell type to a drug.

134. The method of claim 133, wherein said drug is a DNA damaging agent.

135. The method of claim 134, wherein said DNA damaging agent is selected from the group consisting of topoisomerase I inhibitor, topoisomerase II inhibitor, DNA binding agent, and ionizing radiation.

136. The method of claim 135, wherein said DNA damaging agent is selected from the group consisting of doxorubicin, camptothecin, and cisplatin.

137. A method of evaluating the responsiveness of cells of a cell type to treatment of a drug, comprising
(a) contacting one or more cells of said cell type with said drug, wherein said one or more cells express a first small interference RNA (siRNA) targeting a primary target gene, and wherein said one or more cells are subject to treatment of a composition that modulates the expression of one or more secondary target genes and/or the activity of one or more proteins encoded respectively by said one or more secondary target genes;
(b) contacting one or more cells of said cell type with said drug, wherein said one or more cells do not express a small interference RNA (siRNA) targeting said primary target gene, and wherein said one or more cells are subject to treatment of said agent that modulates the expression of a secondary target gene and/or the activity of a protein encoded by said secondary target gene; and
(c) comparing the effect of said drug on said one or more cells measured in step (a) to the effect of said drug on said one or more cells measured in step (b), thereby evaluating the responsiveness of said cells to treatment of said drug.

138. A method for evaluating the responsiveness of cells of a cell type to treatment of a drug, said method comprising
(a) generating a clone of cells of said cell type which express a first small interference RNA (siRNA) targeting a primary target gene;
(b) contacting one or more cells of said clone which express said first siRNA with said drug, wherein said one or more cells are subject to treatment of an agent that modulates the expression of a secondary target gene and/or the activity of a protein encoded by said secondary target gene;
(c) contacting one or more cells of said cell type which do not express a small interference RNA (siRNA) targeting said primary target gene with said drug, wherein said one or more cells are subject to treatment of said agent that modulates the expression of a secondary target gene and/or the activity of a protein encoded by said secondary target gene; and
(d) comparing the effect of said drug on said one or more cells measured in step (b) to the effect of said drug on said one or more cells measured in step (c), thereby evaluating the responsiveness of said cells to treatment of said drug.

139. The method of claim 137 or 138, wherein the effect of said drug on said one or more cells expressing said first siRNA is enhanced as compared to the effect of said drug on a cell of said cell type not expressing said first siRNA.

140. The method of claim 137 or 138, wherein the effect of said drug on said one or more cells expressing said first siRNA is reduced as compared to the effect of said drug on a cell of said cell type not expressing said first siRNA.

141. The method of claim 137 or 138, wherein said composition comprises one or more inhibitors of said one or more secondary target gene.

142. The method of claim 137 or 138, wherein said composition comprises one or more second siRNAs targeting and silencing said one or more secondary target gene.

143. The method of claim 142, wherein said one or more second siRNAs comprises at least k different siRNAs, wherein said k is selected from the group consisting of 2, 3, 4, 5, 6 and 10.

144. The method of claim 143, wherein the total siRNA concentration of said at least k different siRNAs in said agent is an optimal concentration for silencing said secondary target gene, wherein said optimal concentration is a concentration further increase of which does not increase the level of silencing substantially.

145. The method of claim 144, wherein said optimal concentration is a concentration further increase of which does not increase the level of silencing by more than 20%, more than 10%, or more than 5%.

146. The method of claim 144, wherein the concentration of each said at least k different siRNA is about the same.

147. The method of claim 144, wherein the respective concentrations of said at least k different siRNAs are different from each other by less than 50%, less than 20%, or less than 10%.

148. The method of claim 144, wherein none of the siRNAs in said agent has a concentration that is more than 80%, more than 50%, or more than 20% of said total siRNA concentration of said different siRNAs.

149. The method of claim 144, wherein at least one siRNA in said agent has a concentration that is more than 20% or more than 50% of said total siRNA concentration of said at least k different siRNAs.

150. The method of claim 144, wherein the number of different siRNAs and the concentration of each siRNA in said agent is chosen such that said agent causes less than 10%, less than 1%, less than 0.1%, or less than 0.01% of silencing of any off-target genes.

151. The method of claim 137 or 138, wherein said cell type is a cancer cell type, and wherein said primary target gene is p53.

152. The method of claim 138, further comprising a step (e) repeating steps (b)-(d) for each of a plurality of different secondary target genes.

153. The method of claim 137, further comprising a step (d) repeating steps (a)-(b) for each of a plurality of different secondary target genes.

154. The method of claim 152 or 153, wherein said plurality of secondary target genes comprises at least the number of different genes selected from the group consisting of 5, 10, 100, 1,000, and 5,000 different genes.

155. The method of claim 154, wherein said drug is a DNA damaging agent.

156. The method of claim 155, wherein said DNA damaging agent is selected from the group consisting of topoisomerase I inhibitor, topoisomerase II inhibitor, DNA binding agent, and ionizing radiation.

157. The method of claim 156, wherein said DNA damaging agent is selected from the group consisting of doxorubicin, camptothecin, and cisplatin.

158. A method for treating a mammal having a cancer, comprising administering to said mammal a therapeutically sufficient amount of an agent, said agent regulating the expression of a gene and/or activity of a protein encoded by said gene, wherein said mammal is subject to a therapy comprising administering to said mammal a therapeutically sufficient amount of a composition comprising one or more DNA damaging agents.

159. A method for treating a mammal having a cancer, comprising administering to said mammal i) a therapeutically sufficient amount of an agent, said agent regulating the expression of a gene and/or activity of a protein encoded by said gene, and ii) a therapeutically sufficient amount of a composition comprising one or more DNA damaging agents.

160. The method of claim 158 or 159, wherein said agent reduces the expression of said gene in cells of said cancer.

161. The method of claim 158 or 159, wherein said agent enhances the expression of said gene in cells of said cancer.

162. The method of claim 161, wherein said one or more DNA damaging agents are selected from the group consisting of topoisomerase I inhibitor, topoisomerase II inhibitor, DNA binding agent, and ionizing radiation, and wherein said gene is selected from the group consisting of EPHB3, WEE1, ELK1, STK6, BRCA1, BRCA2, BARD1, and RAD51.

163. The method of claim 161, wherein said one or more DNA damaging agents are selected from the group consisting of doxorubicin, camptothecin, and cisplatin, and wherein said gene is selected from the group consisting of EPHB3, Wee1, ELK1, STK6, BRCA1, BRCA2, BARD1, and RAD51.

164. The method of claim 163, wherein said agent comprises an siRNA targeting said gene.

165. A method for evaluating sensitivity of a cell to the growth inhibitory effect of an agent, said method comprising determining a transcript level of each of one or more genes in said cell, wherein each said transcript level below a predetermined threshold level for a respective gene indicates that said cell is sensitive to the growth inhibitory effect of said DNA damaging agent.

166. The method of claim 165, wherein said agent is a DNA damaging agent selected from the group consisting of topoisomerase I inhibitor, topoisomerase II inhibitor, DNA binding agent, and ionizing radiation, and wherein said gene is selected from the group consisting of EPHB3, WEE1, ELK1, STK6, BRCA1, BRCA2, BARD1, and RAD51.

167. The method of claim 165, wherein said DNA damaging agent is selected from the group consisting of doxorubicin, camptothecin, and cisplatin, and wherein said gene is selected from the group consisting of EPHB3, WEE1, ELK1, STK6, BRCA1, BRCA2, BARD1, and RAD51.

168. The method of any one of claims 166-167, wherein said one or more genes comprises at least about 5 to about 50 different genes.

169. The method of claim 168, wherein each said transcript level is a 1.5-fold, 2-fold or 3-fold reduction from said threshold level.

170. The method of any one of claims 166-167, wherein said transcript level of said gene is determined by a method comprising measuring the transcript level of said gene using one or more polynucleotide probes, each of said one or more polynucleotide probes comprising a nucleotide sequence in said gene.

171. The method of claim 170, wherein said one or more polynucleotide probes are polynucleotide probes on a microarray.

172. A method for evaluating sensitivity of a cell to the growth inhibitory effect of a DNA damaging agent, said method comprising determining a level of abundance of a protein encoded by a gene in said cell, wherein said level of abundance of said protein below a predetermined threshold level indicates that said cell is sensitive to the growth inhibitory effect of said DNA damaging agent.

173. A method for evaluating sensitivity of a cell to the growth inhibitory effect of a DNA damaging agent, said method comprising determining a level of activity of a protein encoded by a gene in said cell, wherein said activity level above a predetermined threshold level indicates that said cell is sensitive to the growth inhibitory effect of said DNA damaging agent.

174. The method of claim 172 or 173, wherein said DNA damaging agent is selected from the group consisting of topoisomerase I inhibitor, topoisomerase II inhibitor, DNA binding agent, and ionizing radiation, and wherein said gene is selected from the group consisting of EPHB3, Wee1, ELK1, STK6, BRCA1, BRCA2, BARD1, and RAD51.

175. The method of claim 174, wherein said DNA damaging agent is selected from the group consisting of doxorubicin, camptothecin, and cisplatin, and wherein said gene is selected from the group consisting of EPHB3, WEE1, ELK1, STK6, BRCA1, BRCA2, BARD1, and RAD51.

176. The method of claim 172 or 173, wherein said cell is a human cell.

177. A method for regulating sensitivity of a cell to DNA damage, comprising contacting said cell with a sufficient amount of an agent, said agent regulating the expression of a gene selected from the group consisting of EPHB3, WEE1, ELK1, STK6, BRCA1, BRCA2, BARD1, and RAD51 and/or the activity of a protein encoded by said gene.

178. The method of claim 177, wherein said DNA damage is caused by a DNA damaging agent.

179. The method of claim 178, wherein said DNA damaging agent is selected from the group consisting of topoisomerase I inhibitor, topoisomerase II inhibitor, DNA binding agent, and ionizing radiation.

180. The method of claim 179, wherein said DNA damaging agent is selected from the group consisting of doxorubicin, camptothecin, and cisplatin.

181. A method for regulating growth of a cell, comprising contacting said cell with i) a sufficient amount of an agent that regulates the expression of a gene selected from the group consisting of EPHB3, WEE1, ELK1, STK6, BRCA1, BRCA2, BARD1, and RAD51 and/or the activity of a protein encoded by said gene; and ii) a sufficient amount of a DNA damaging agent.

182. The method of claim 177 or 181, wherein said agent reduces the expression of said gene in said cell.

183. The method of claim 177 or 181, wherein said agent comprises an siRNA targeting said gene.

184. The method of claim 183, wherein said agent comprises 2, 3, 4, 5, 6, or 10 different siRNAs targeting said gene.

185. The method of claim 184, wherein the total siRNA concentration of said different siRNAs in said agent is an optimal concentration for silencing said gene, wherein said optimal concentration is a concentration further increase of which does not increase the level of silencing substantially.

186. The method of claim 185, wherein said optimal concentration is a concentration further increase of which does not increase the level of silencing by more than 20%, more than 10%, or more than 5%.

187. The method of claim 185, wherein the concentration of each said different siRNA is about the same.

188. The method of claim 185, wherein the respective concentrations of said different siRNAs are different from each other by less than 50%, less than 20%, or less than 10%.

189. The method of claim 185, wherein none of the siRNAs in said agent has a concentration that is more than 80%, more than 50%, or more than 20% of said total siRNA concentration of said different siRNAs.

190. The method of claim 185, wherein at least one siRNA in said agent has a concentration that is more than 20% or more than 50% of said total siRNA concentration of said different siRNAs.

191. The method of claim 185, wherein the number of different siRNAs and the concentration of each siRNA in said agent is chosen such that said agent causes less than 10%, less than 1%, less than 0.1%, or less than 0.01% of silencing of any off-target genes.

192. A method of identifying an agent that is capable of regulating sensitivity of a cell to the growth inhibitory effect of a DNA damaging agent, wherein said agent is capable of modulating the expression of a gene selected from the group consisting of EPHB3, WEE1, ELK1, STK6, BRCA1, BRCA2, BARD1, and RAD51 and/or the activity of a protein encoded by said gene, said method comprising comparing inhibitory effect of said DNA damaging agent on cells expressing said gene in the presence of said agent with inhibitory effect of said DNA damaging agent on cells expressing said gene in the absence of said agent, wherein a difference in said inhibitory effect of said DNA damaging agent identifies said agent as capable of regulating sensitivity of said cell to the growth inhibitory effect of said DNA damaging agent.

193. A method of identifying an agent that is capable of regulating sensitivity of a cell to the growth inhibitory effect of a DNA damaging agent, wherein said agent is capable of modulating the expression of a gene selected from the group consisting of EPHB3, WEE1, ELK1, STK6, BRCA1, BRCA2, BARD1, and RAD51 and/or activity of a protein encoded by said gene, said method comprising:
(a) contacting a first cell expressing said gene with said DNA damaging agent in the presence of said agent and measuring a first growth inhibitory effect;
(b) contacting a second cell expressing said gene with said DNA damaging agent in the absence of said agent and measuring a second growth inhibitory effect; and
(c) comparing said first and second inhibitory effects measured in said step (a) and (b),
wherein a difference between said first and second inhibitory effects identifies said agent as capable of regulating sensitivity of a cell to the growth inhibitory effect of said DNA damaging agent.

194. The method of claim 192 or 193, wherein said cell expresses an siRNA targeting a primary target gene.

195. The method of claim 194, wherein said primary target gene is p53.

196. The method of claim 192 or 193, wherein said agent comprises a molecule that reduces expression of said gene.

197. The method of claim 196, wherein said agent comprises an siRNA targeting said gene.

198. The method of claim 197, wherein said agent comprises 2, 3, 4, 5, 6, or 10 different siRNAs targeting said gene.

199. The method of claim 198, wherein the total siRNA concentration of said different siRNAs in said agent is an optimal concentration for silencing said gene, wherein said optimal concentration is a concentration further increase of which does not increase the level of silencing substantially.

200. The method of claim 199, wherein said optimal concentration is a concentration further increase of which does not increase the level of silencing by more than 20%, more than 10%, or more than 5%.

201. The method of claim 199, wherein the concentration of each said different siRNA is about the same.

202. The method of claim 199, wherein the respective concentrations of said different siRNAs are different from each other by less than 50%, less than 20%, or less than 10%.

203. The method of claim 199, wherein none of the siRNAs in said agent has a concentration that is more than 80%, more than 50%, or more than 20% of said total siRNA concentration of said different siRNAs.

204. The method of claim 199, wherein at least one siRNA in said agent has a concentration that is more than 20% or more than 50% of said total siRNA concentration of said different siRNAs.

205. The method of claim 199, wherein the number of different siRNAs and the concentration of each siRNA in said agent is chosen such that said agent causes less than 10%, less than 1%, less than 0.1%, less than 0.01% of silencing of any off-target genes.

206. A cell comprising one or more different small interfering RNAs (siRNAs) targeting a gene selected from the group consisting of EPHB3, Wee1, ELK1, BRCA1, BRCA2, BARD1, and RAD51 in said cell.

207. The cell of claim 206, wherein said one or more different siRNAs comprises 2, 3, 4, 5, 6, or 10 different siRNAs.

208. The cell of claim 206, wherein said cell is a human cell.

209. The cell of claim 208, wherein said cell is a murine cell.

210. A microarray for diagnosing sensitivity of a cell to the growth inhibitory effect of a DNA damaging agent, said microarray comprising one or more polynucleotide probes, wherein each said polynucleotide probe comprises a nucleotide sequence in one or more genes selected from the group consisting of EPHB3, WEE1, ELK1, STK6, BRCA1, BRCA2, BARD1, and RAD51.

211. A kit for diagnosis of sensitivity of a cell to the growth inhibitory effect of a DNA damaging agent, comprising in one or more containers one or more polynucleotide probes, wherein each said polynucleotide probe comprises a nucleotide sequence in a gene selected from the group consisting of EPHB3, WEE1, ELK1, STK6, BRCA1, BRCA2, BARD1, and RAD51.

212. A kit for screening for agents which regulate sensitivity of a cell to the growth inhibitory effect of a DNA damaging agent, comprising in one or more containers (i) the cell of any one of claims 206-211; and (ii) said DNA damaging agent.

213. A kit for treating a mammal having a cancer, comprising in one or more containers (i) a sufficient amount of an agent that regulates the expression of a gene selected from the group consisting of EPHB3, WEE1, ELK1, STK6, BRCA1, BRCA2, BARD1, and RAD51 and/or the activity of a protein encoded by said gene; and (ii) a DNA damaging agent.

214. The method of any one of claims 192-193, wherein said DNA damaging agent is selected from the group consisting of a topoisomerase I inhibitor, a topoisomerase II inhibitor, a DNA binding agent, and ionizing radiation.

215. The method of claim 214, wherein said DNA damaging agent is selected from the group consisting of doxorubicin, camptothecin, and cisplatin.

216. The kit of claim 212, wherein said DNA damaging agent is selected from the group consisting of a topoisomerase I inhibitor, a topoisomerase II inhibitor, a DNA binding agent, and ionizing radiation.

217. The method of claim 216, wherein said DNA damaging agent is selected from the group consisting of doxorubicin, camptothecin, and cisplatin.

218. The method of claim 21, 117, 137 or 138, wherein level of silencing of said primary target gene is controlled.


SEQUENCE LISTING (1239 sequences)

SEQ ID NO: 1 - DNA, 21 bp, Artificial Sequence
  siRNA with 19 paired ribonucleotides and two unpaired nucleotides(“t”) on the 3′ end of each strand used in silencing TPX2
gcacaaaagcuugucuccatt

SEQ ID NO: 2 - DNA, 21 bp, Artificial Sequence
  siRNA with 19 paired ribonucleotides and two unpaired nucleotides(“t”) on the 3′ end of each strand used in silencing TPX2
uugcagauuuugggugguctt

SEQ ID NO: 3 - DNA, 21 bp, Artificial Sequence
  siRNA with 19 paired ribonucleotides and two unpaired nucleotides(“t”) on the 3′ end of each strand used in silencing TPX2
acagucuuaggaaucgugctt

SEQ ID NO: 4 - DNA, 21 bp, Artificial Sequence
  siRNA with 19 paired ribonucleotides and two unpaired nucleotides(“t”) on the 3′ end of each strand used in silencing TPX2
ccucccuauucagaaagcutt

SEQ ID NO: 5 - DNA, 21 bp, Artificial Sequence
  siRNA with 19 paired ribonucleotides and two unpaired nucleotides(“t”) on the 3′ end of each strand used in silencing TPX2
gacuuugaaauuggucgcctt

SEQ ID NO: 6 - DNA, 21 bp, Artificial Sequence
  siRNA with 19 paired ribonucleotides and two unpaired nucleotides(“t”) on the 3′ end of each strand used in silencing TPX2
cacccaaaagagcaagcagtt

SEQ ID NO: 7 - DNA, 21 bp, Artificial Sequence
  siRNA with 19 paired ribonucleotides and two unpaired nucleotides(“t”) on the 3′ end of each strand used in silencing TPX2
aaccagucuauuagacggctt

SEQ ID NO: 8 - DNA, 21 bp, Artificial Sequence
  siRNA with 19 paired ribonucleotides and two unpaired nucleotides(“t”) on the 3′ end of each strand used in silencing TPX2
gugacucuccaucuuguagtt

SEQ ID NO: 9 - DNA, 21 bp, Artificial Sequence
  siRNA with 19 paired ribonucleotides and two unpaired nucleotides(“t”) on the 3′ end of each strand used in silencing TPX2
guggccucaaaggcacuuatt

SEQ ID NO: 10 - DNA, 21 bp, Artificial Sequence
  siRNA with 19 paired ribonucleotides and two unpaired nucleotides(“t”) on the 3′ end of each strand used in silencing TPX2
cccaucaccucaguuguuutt

SEQ ID NO: 11 - DNA, 21 bp, Artificial Sequence
  siRNA with 19 paired ribonucleotides and two unpaired nucleotides(“t”) on the 3′ end of each strand used in silencing TPX2
gaccugccguuacauuccutt

SEQ ID NO: 12 - DNA, 21 bp, Artificial Sequence
  siRNA with 19 paired ribonucleotides and two unpaired nucleotides(“t”) on the 3′ end of each strand used in silencing TPX2
ggagaaccagucugaaaactt

SEQ ID NO: 13 - DNA, 21 bp, Artificial Sequence
  siRNA with 19 paired ribonucleotides and two unpaired nucleotides(“t”) on the 3′ end of each strand used in silencing TPX2
augcuggaaauugcccugctt

SEQ ID NO: 14 - DNA, 21 bp, Artificial Sequence
  siRNA with 19 paired ribonucleotides and two unpaired nucleotides(“t”) on the 3′ end of each strand used in silencing TPX2
acaacccagaggacugguutt

SEQ ID NO: 15 - DNA, 21 bp, Artificial Sequence
  siRNA with 19 paired ribonucleotides and two unpaired nucleotides(“t”) on the 3′ end of each strand used in silencing TPX2
uauguucugggccaacuugtt

SEQ ID NO: 16 - DNA, 21 bp, Artificial Sequence
  siRNA with 19 paired ribonucleotides and two unpaired nucleotides(“t”) on the 3′ end of each strand used in silencing TPX2
ccagauccuugucuggaagtt

SEQ ID NO: 17 - DNA, 21 bp, Artificial Sequence
  siRNA with 19 paired ribonucleotides and two unpaired nucleotides(“t”) on the 3′ end of each strand used in silencing TPX2
cgacaacaagcugcugguctt

SEQ ID NO: 18 - DNA, 21 bp, Artificial Sequence
  siRNA with 19 paired ribonucleotides and two unpaired nucleotides(“t”) on the 3′ end of each strand used in silencing TPX2
gaagcuguccauguuggagtt

SEQ ID NO: 19 - DNA, 21 bp, Artificial Sequence
  siRNA with 19 paired ribonucleotides and two unpaired nucleotides(“t”) on the 3′ end of each strand used in silencing TPX2
cuguaugggguauucgcugtt

SEQ ID NO: 20 - DNA, 21 bp, Artificial Sequence
  siRNA with 19 paired ribonucleotides and two unpaired nucleotides(“t”) on the 3′ end of each strand used in silencing TPX2
acccauuugccagcucaagtt

SEQ ID NO: 21 - DNA, 21 bp, Artificial Sequence
  siRNA with 19 paired ribonucleotides and two unpaired nucleotides(“t”) on the 3′ end of each strand used in silencing TPX2
cagacuccauguuugcagutt

SEQ ID NO: 22 - DNA, 21 bp, Artificial Sequence
  siRNA with 19 paired ribonucleotides and two unpaired nucleotides(“t”) on the 3′ end of each strand used in silencing TPX2
uacauuugccacagguggutt

SEQ ID NO: 23 - DNA, 21 bp, Artificial Sequence
  siRNA with 19 paired ribonucleotides and two unpaired nucleotides(“t”) on the 3′ end of each strand used in silencing TPX2
caauucguacuccccaaugtt

SEQ ID NO: 24 - DNA, 21 bp, Artificial Sequence
  siRNA with 19 paired ribonucleotides and two unpaired nucleotides(“t”) on the 3′ end of each strand used in silencing TPX2
agcugcuucagacugcuuctt

SEQ ID NO: 25 - DNA, 21 bp, Artificial Sequence
  siRNA with 19 paired ribonucleotides and two unpaired nucleotides(“t”) on the 3′ end of each strand used in silencing TPX2
gaccuuuccagauucguggtt

SEQ ID NO: 26 - DNA, 21 bp, Artificial Sequence
  siRNA with 19 paired ribonucleotides and two unpaired nucleotides(“t”) on the 3′ end of each strand used in silencing TPX2
agagcagagcagauccguutt

SEQ ID NO: 27 - DNA, 21 bp, Artificial Sequence
  siRNA with 19 paired ribonucleotides and two unpaired nucleotides(“t”) on the 3′ end of each strand used in silencing TPX2
ccagcggcucaaggagguutt

SEQ ID NO: 28 - DNA, 21 bp, Artificial Sequence
  siRNA with 19 paired ribonucleotides and two unpaired nucleotides(“t”) on the 3′ end of each strand used in silencing TPX2
ccaugacgucggacauuuutt

SEQ ID NO: 29 - DNA, 21 bp, Artificial Sequence
  siRNA with 19 paired ribonucleotides and two unpaired nucleotides(“t”) on the 3′ end of each strand used in silencing TPX2
gugcucuuaucgccucugutt

SEQ ID NO: 30 - DNA, 21 bp, Artificial Sequence
  siRNA with 19 paired ribonucleotides and two unpaired nucleotides(“t”) on the 3′ end of each strand used in silencing TPX2
acgcaagaaguacaacgugtt

SEQ ID NO: 31 - DNA, 21 bp, Artificial Sequence
  siRNA with 19 paired ribonucleotides and two unpaired nucleotides(“t”) on the 3′ end of each strand used in silencing TPX2
gcaaguugagcucuaccgctt

SEQ ID NO: 32 - DNA, 21 bp, Artificial Sequence
  siRNA with 19 paired ribonucleotides and two unpaired nucleotides(“t”) on the 3′ end of each strand used in silencing TPX2
uggccagcgcuuacuggaatt

SEQ ID NO: 33 - DNA, 21 bp, Artificial Sequence
  siRNA with 19 paired ribonucleotides and two unpaired nucleotides(“t”) on the 3′ end of each strand used in silencing TPX2
ggccaaggaggcgcuggaatt

SEQ ID NO: 34 - DNA, 21 bp, Artificial Sequence
  siRNA with 19 paired ribonucleotides and two unpaired nucleotides(“t”) on the 3′ end of each strand used in silencing TPX2
augacccucuggauguuugtt

SEQ ID NO: 35 - DNA, 21 bp, Artificial Sequence
  siRNA with 19 paired ribonucleotides and two unpaired nucleotides(“t”) on the 3′ end of each strand used in silencing TPX2
ugccaaugaugaggccacatt

SEQ ID NO: 36 - DNA, 21 bp, Artificial Sequence
  siRNA with 19 paired ribonucleotides and two unpaired nucleotides(“t”) on the 3′ end of each strand used in silencing TPX2
gaaagaacaggugaucagctt

SEQ ID NO: 37 - DNA, 21 bp, Artificial Sequence
  siRNA with 19 paired ribonucleotides and two unpaired nucleotides(“t”) on the 3′ end of each strand used in silencing TPX2
cguacgcggaauacuucgatt

SEQ ID NO: 38 - DNA, 21 bp, Artificial Sequence
  siRNA with 19 paired ribonucleotides and two unpaired nucleotides(“t”) on the 3′ end of each strand used in silencing TPX2
cuggaucguaagaaggcagtt

SEQ ID NO: 39 - DNA, 21 bp, Artificial Sequence
  siRNA with 19 paired ribonucleotides and two unpaired nucleotides(“t”) on the 3′ end of each strand used in silencing TPX2
ggacaacugcagcuacucutt

SEQ ID NO: 40 - DNA, 21 bp, Artificial Sequence
  siRNA with 19 paired ribonucleotides and two unpaired nucleotides(“t”) on the 3′ end of each strand used in silencing TPX2
uggaggggaaugcucagaatt

SEQ ID NO: 41 - DNA, 21 bp, Artificial Sequence
  siRNA with 19 paired ribonucleotides and two unpaired nucleotides(“t”) on the 3′ end of each strand used in silencing TPX2
uaaagauggcacuuucccgtt

SEQ ID NO: 42 - DNA, 21 bp, Artificial Sequence
  siRNA with 19 paired ribonucleotides and two unpaired nucleotides(“t”) on the 3′ end of each strand used in silencing TPX2
aaggcagcuaaaggaagugtt

SEQ ID NO: 43 - DNA, 19 bp, Artificial Sequence
  pRS-p53 1026 19mer sequence
gactccagtggtaatctac

SEQ ID NO: 44 - DNA, 19 bp, Artificial Sequence
  forward primer
gtagattaccactggagtc

SEQ ID NO: 45 - DNA, 23 bp, Artificial Sequence
  Reverse primer
cccttgaacctcctcgttcgacc

SEQ ID NO: 46 - DNA, 19 bp, Artificial Sequence
  pRS-STK6 2178 19mer sequence
cattggagtcatagcatgt

SEQ ID NO: 47 - DNA, 21 bp, Artificial Sequence
  siRNA with 19 paired ribonucleotides and two unpaired nucleotides(“t”) on the 3′ end of each strand used in silencing TPX2
gaccugugccuuuuagagatt

SEQ ID NO: 48 - DNA, 21 bp, Artificial Sequence
  siRNA with 19 paired ribonucleotides and two unpaired nucleotides(“t”) on the 3′ end of each strand used in silencing TPX2
gacuucauugacaguggcctt

SEQ ID NO: 49 - DNA, 21 bp, Artificial Sequence
  siRNA with 19 paired ribonucleotides and two unpaired nucleotides(“t”) on the 3′ end of each strand used in silencing TPX2
aaaggacaacugcagcuactt

SEQ ID NO: 50 - DNA, 21 bp, Artificial Sequence
  siRNA with 19 paired ribonucleotides and two unpaired nucleotides(“t”) on the 3′ end of each strand used in screens of DNA damaging agents cisplatin screen
aaaggcugcucacaaguuctt

SEQ ID NO: 51 - DNA, 21 bp, Artificial Sequence
  siRNA with 19 paired ribonucleotides and two unpaired nucleotides(“t”) on the 3′ end of each strand used in screens of DNA damaging agents cisplatin screen
agcugcucaacaaaccagatt

SEQ ID NO: 52 - DNA, 21 bp, Artificial Sequence
  siRNA with 19 paired ribonucleotides and two unpaired nucleotides(“t”) on the 3′ end of each strand used in screens of DNA damaging agents cisplatin screen
augaggaacagggcaauagtt

SEQ ID NO: 53 - DNA, 21 bp, Artificial Sequence
  siRNA with 19 paired ribonucleotides and two unpaired nucleotides(“t”) on the 3′ end of each strand used in screens of DNA damaging agents cisplatin screen
gaauggggucaacgauauctt

SEQ ID NO: 54 - DNA, 21 bp, Artificial Sequence
  siRNA with 19 paired ribonucleotides and two unpaired nucleotides(“t”) on the 3′ end of each strand used in screens of DNA damaging agents cisplatin screen
ggacgagacuuccucuugatt

SEQ ID NO: 55 - DNA, 21 bp, Artificial Sequence
  siRNA with 19 paired ribonucleotides and two unpaired nucleotides(“t”) on the 3′ end of each strand used in screens of DNA damaging agents cisplatin screen
guguggcaaggaguuuucutt

SEQ ID NO: 56 - DNA, 21 bp, Artificial Sequence
  siRNA with 19 paired ribonucleotides and two unpaired nucleotides(“t”) on the 3′ end of each strand used in screens of DNA damaging agents cisplatin screen
gaauccuaagaagaggccgtt

SEQ ID NO: 57 - DNA, 21 bp, Artificial Sequence
  siRNA with 19 paired ribonucleotides and two unpaired nucleotides(“t”) on the 3′ end of each strand used in screens of DNA damaging agents cisplatin screen
gaggaggucuuucauugggtt

SEQ ID NO: 58 - DNA, 21 bp, Artificial Sequence
  siRNA with 19 paired ribonucleotides and two unpaired nucleotides(“t”) on the 3′ end of each strand used in screens of DNA damaging agents cisplatin screen
gauagucaagcuagacccatt

SEQ ID NO: 59 - DNA, 21 bp, Artificial Sequence
  siRNA with 19 paired ribonucleotides and two unpaired nucleotides(“t”) on the 3′ end of each strand used in screens of DNA damaging agents cisplatin screen
auccuccuguaauggaacctt

SEQ ID NO: 60 - DNA, 21 bp, Artificial Sequence
  siRNA with 19 paired ribonucleotides and two unpaired nucleotides(“t”) on the 3′ end of each strand used in screens of DNA damaging agents cisplatin screen
gaagaggacaggauugucgtt

SEQ ID NO: 61 - DNA, 21 bp, Artificial Sequence
  siRNA with 19 paired ribonucleotides and two unpaired nucleotides(“t”) on the 3′ end of each strand used in screens of DNA damaging agents cisplatin screen
gaccaacagcagagauaugtt

SEQ ID NO: 62 - DNA, 21 bp, Artificial Sequence
  siRNA with 19 paired ribonucleotides and two unpaired nucleotides(“t”) on the 3′ end of each strand used in screens of DNA damaging agents cisplatin screen
accagagaccaaaugucactt

SEQ ID NO: 63 - DNA, 21 bp, Artificial Sequence
  siRNA with 19 paired ribonucleotides and two unpaired nucleotides(“t”) on the 3′ end of each strand used in screens of DNA damaging agents cisplatin screen
auaagcccaccagcuugugtt

SEQ ID NO: 64 - DNA, 21 bp, Artificial Sequence
  siRNA with 19 paired ribonucleotides and two unpaired nucleotides(“t”) on the 3′ end of each strand used in screens of DNA damaging agents cisplatin screen
ucaacaccgcuuugccgautt

SEQ ID NO: 65 - DNA, 21 bp, Artificial Sequence
  siRNA with 19 paired ribonucleotides and two unpaired nucleotides(“t”) on the 3′ end of each strand used in screens of DNA damaging agents cisplatin screen
acuauagacuuccgcaacctt

SEQ ID NO: 66 - DNA, 21 bp, Artificial Sequence
  siRNA with 19 paired ribonucleotides and two unpaired nucleotides(“t”) on the 3′ end of each strand used in screens of DNA damaging agents cisplatin screen
caguagauugcuguggccutt

SEQ ID NO: 67 - DNA, 21 bp, Artificial Sequence
  siRNA with 19 paired ribonucleotides and two unpaired nucleotides(“t”) on the 3′ end of each strand used in screens of DNA damaging agents cisplatin screen
cuccauacagcuucugaagtt

SEQ ID NO: 68 - DNA, 21 bp, Artificial Sequence
  siRNA with 19 paired ribonucleotides and two unpaired nucleotides(“t”) on the 3′ end of each strand used in screens of DNA damaging agents cisplatin screen
ucacuuagcagcugagucutt

SEQ ID NO: 69 - DNA, 21 bp, Artificial Sequence
  siRNA with 19 paired ribonucleotides and two unpaired nucleotides(“t”) on the 3′ end of each strand used in screens of DNA damaging agents cisplatin screen
uugacagcacuggucagagtt

SEQ ID NO: 70 - DNA, 21 bp, Artificial Sequence
  siRNA with 19 paired ribonucleotides and two unpaired nucleotides(“t”) on the 3′ end of each strand used in screens of DNA damaging agents cisplatin screen
uuggcaagaacuucuuggctt

SEQ ID NO: 71 - DNA, 21 bp, Artificial Sequence
  siRNA with 19 paired ribonucleotides and two unpaired nucleotides(“t”) on the 3′ end of each strand used in screens of DNA damaging agents cisplatin screen
acaaaaacggagauccguctt

SEQ ID NO: 72 - DNA, 21 bp, Artificial Sequence
  siRNA with 19 paired ribonucleotides and two unpaired nucleotides(“t”) on the 3′ end of each strand used in screens of DNA damaging agents cisplatin screen
auaagcagcaagaaacggctt

SEQ ID NO: 73 - DNA, 21 bp, Artificial Sequence
  siRNA with 19 paired ribonucleotides and two unpaired nucleotides(“t”) on the 3′ end of each strand used in screens of DNA damaging agents cisplatin screen
gaauuucgggcuacuuuggtt

SEQ ID NO: 74 - DNA, 21 bp, Artificial Sequence
  siRNA with 19 paired ribonucleotides and two unpaired nucleotides(“t”) on the 3′ end of each strand used in screens of DNA damaging agents cisplatin screen
gaaaaugaagcuuugcgggtt

SEQ ID NO: 75 - DNA, 21 bp, Artificial Sequence
  siRNA with 19 paired ribonucleotides and two unpaired nucleotides(“t”) on the 3′ end of each strand used in screens of DNA damaging agents cisplatin screen
gaagagaucccagugcuuctt

SEQ ID NO: 76 - DNA, 21 bp, Artificial Sequence
  siRNA with 19 paired ribonucleotides and two unpaired nucleotides(“t”) on the 3′ end of each strand used in screens of DNA damaging agents cisplatin screen
ucugaaagugaccagcucatt

SEQ ID NO: 77 - DNA, 21 bp, Artificial Sequence
  siRNA with 19 paired ribonucleotides and two unpaired nucleotides(“t”) on the 3′ end of each strand used in screens of DNA damaging agents cisplatin screen
ccugcagcaacugauuacctt

SEQ ID NO: 78 - DNA, 21 bp, Artificial Sequence
  siRNA with 19 paired ribonucleotides and two unpaired nucleotides(“t”) on the 3′ end of each strand used in screens of DNA damaging agents cisplatin screen
gaacuugagaagaugcgagtt

SEQ ID NO: 79 - DNA, 21 bp, Artificial Sequence
  siRNA with 19 paired ribonucleotides and two unpaired nucleotides(“t”) on the 3′ end of each strand used in screens of DNA damaging agents cisplatin screen
gaagaggcccacugaaguutt

SEQ ID NO: 80 - DNA, 21 bp, Artificial Sequence
  siRNA with 19 paired ribonucleotides and two unpaired nucleotides(“t”) on the 3′ end of each strand used in screens of DNA damaging agents cisplatin screen
caaaugggcaggacucuuatt

SEQ ID NO: 81 - DNA, 21 bp, Artificial Sequence
  siRNA with 19 paired ribonucleotides and two unpaired nucleotides(“t”) on the 3′ end of each strand used in screens of DNA damaging agents cisplatin screen
cuguucagcccaguuugaatt

SEQ ID NO: 82 - DNA, 21 bp, Artificial Sequence
  siRNA with 19 paired ribonucleotides and two unpaired nucleotides(“t”) on the 3′ end of each strand used in screens of DNA damaging agents cisplatin screen
ucuccaaggaaguuguacctt

SEQ ID NO: 83 - DNA, 21 bp, Artificial Sequence
  siRNA with 19 paired ribonucleotides and two unpaired nucleotides(“t”) on the 3′ end of each strand used in screens of DNA damaging agents cisplatin screen
accaaguauccgcaaaaggtt

SEQ ID NO: 84 - DNA, 21 bp, Artificial Sequence
  siRNA with 19 paired ribonucleotides and two unpaired nucleotides(“t”) on the 3′ end of each strand used in screens of DNA damaging agents cisplatin screen
agaccuguauuaguacgcctt

SEQ ID NO: 85 - DNA, 21 bp, Artificial Sequence
  siRNA with 19 paired ribonucleotides and two unpaired nucleotides(“t”) on the 3′ end of each strand used in screens of DNA damaging agents cisplatin screen
caagcuuuacccagccugutt

SEQ ID NO: 86 - DNA, 21 bp, Artificial Sequence
  siRNA with 19 paired ribonucleotides and two unpaired nucleotides(“t”) on the 3′ end of each strand used in screens of DNA damaging agents cisplatin screen
gaaacugcagcuaucuucctt

SEQ ID NO: 87 - DNA, 21 bp, Artificial Sequence
  siRNA with 19 paired ribonucleotides and two unpaired nucleotides(“t”) on the 3′ end of each strand used in screens of DNA damaging agents cisplatin screen
guuacaaugaggcugaugctt

SEQ ID NO: 88 - DNA, 21 bp, Artificial Sequence
  siRNA with 19 paired ribonucleotides and two unpaired nucleotides(“t”) on the 3′ end of each strand used in screens of DNA damaging agents cisplatin screen
ucacgacucgcugaacugutt

SEQ ID NO: 89 - DNA, 21 bp, Artificial Sequence
  siRNA with 19 paired ribonucleotides and two unpaired nucleotides(“t”) on the 3′ end of each strand used in screens of DNA damaging agents cisplatin screen
acuuaggugaagcagcauctt

SEQ ID NO: 90 - DNA, 21 bp, Artificial Sequence
  siRNA with 19 paired ribonucleotides and two unpaired nucleotides(“t”) on the 3′ end of each strand used in screens of DNA damaging agents cisplatin screen
gggcagugaagacuugauutt

SEQ ID NO: 91 - DNA, 21 bp, Artificial Sequence
  siRNA with 19 paired ribonucleotides and two unpaired nucleotides(“t”) on the 3′ end of each strand used in screens of DNA damaging agents cisplatin screen
ugaagugggcuccaguauutt

SEQ ID NO: 92 - DNA, 21 bp, Artificial Sequence
  siRNA with 19 paired ribonucleotides and two unpaired nucleotides(“t”) on the 3′ end of each strand used in screens of DNA damaging agents cisplatin screen
acaucguggugcgagguuatt

SEQ ID NO: 93 - DNA, 21 bp, Artificial Sequence
  siRNA with 19 paired ribonucleotides and two unpaired nucleotides(“t”) on the 3′ end of each strand used in screens of DNA damaging agents cisplatin screen
augagccgccaauuggacatt

SEQ ID NO: 94 - DNA, 21 bp, Artificial Sequence
  siRNA with 19 paired ribonucleotides and two unpaired nucleotides(“t”) on the 3′ end of each strand used in screens of DNA damaging agents cisplatin screen
auggcaaguuuggaaggactt

SEQ ID NO: 95 - DNA, 21 bp, Artificial Sequence
  siRNA with 19 paired ribonucleotides and two unpaired nucleotides(“t”) on the 3′ end of each strand used in screens of DNA damaging agents cisplatin screen
acggcguuuaucuucgcuatt

SEQ ID NO: 96 - DNA, 21 bp, Artificial Sequence
  siRNA with 19 paired ribonucleotides and two unpaired nucleotides(“t”) on the 3′ end of each strand used in screens of DNA damaging agents cisplatin screen
cccucuugccauccugaugtt

SEQ ID NO: 97 - DNA, 21 bp, Artificial Sequence
  siRNA with 19 paired ribonucleotides and two unpaired nucleotides(“t”) on the 3′ end of each strand used in screens of DNA damaging agents cisplatin screen
cuauuuauugugcuggguctt

SEQ ID NO: 98 - DNA, 21 bp, Artificial Sequence
  siRNA with 19 paired ribonucleotides and two unpaired nucleotides(“t”) on the 3′ end of each strand used in screens of DNA damaging agents cisplatin screen
aucgauucugcuccucuagtt

SEQ ID NO: 99 - DNA, 21 bp, Artificial Sequence
  siRNA with 19 paired ribonucleotides and two unpaired nucleotides(“t”) on the 3′ end of each strand used in screens of DNA damaging agents cisplatin screen
cugaagaagcagucgcagutt

SEQ ID NO: 100 - DNA, 21 bp, Artificial Sequence
  siRNA with 19 paired ribonucleotides and two unpaired nucleotides(“t”) on the 3′ end of each strand used in screens of DNA damaging agents cisplatin screen
ugccugaaagagacuugugtt

SEQ ID NO: 101 - DNA, 21 bp, Artificial Sequence
  siRNA with 19 paired ribonucleotides and two unpaired nucleotides(“t”) on the 3′ end of each strand used in screens of DNA damaging agents cisplatin screen
aucggcucuggagaauuugtt

SEQ ID NO: 102 - DNA, 21 bp, Artificial Sequence
  siRNA with 19 paired ribonucleotides and two unpaired nucleotides(“t”) on the 3′ end of each strand used in screens of DNA damaging agents cisplatin screen
caaggaucuccaguccacatt

SEQ ID NO: 103 - DNA, 21 bp, Artificial Sequence
  siRNA with 19 paired ribonucleotides and two unpaired nucleotides(“t”) on the 3′ end of each strand used in screens of DNA damaging agents cisplatin screen
uguaccuguguguccaucutt

SEQ ID NO: 104 - DNA, 21 bp, Artificial Sequence
  siRNA with 19 paired ribonucleotides and two unpaired nucleotides(“t”) on the 3′ end of each strand used in screens of DNA damaging agents cisplatin screen
aggacacuuuggguaccagtt

SEQ ID NO: 105 - DNA, 21 bp, Artificial Sequence
  siRNA with 19 paired ribonucleotides and two unpaired nucleotides(“t”) on the 3′ end of each strand used in screens of DNA damaging agents cisplatin screen
gacccuaaagaucguccuutt

SEQ ID NO: 106 - DNA, 21 bp, Artificial Sequence
  siRNA with 19 paired ribonucleotides and two unpaired nucleotides(“t”) on the 3′ end of each strand used in screens of DNA damaging agents cisplatin screen
gcugaggagaauaugccuctt

SEQ ID NO: 107 - DNA, 21 bp, Artificial Sequence
  siRNA with 19 paired ribonucleotides and two unpaired nucleotides(“t”) on the 3′ end of each strand used in screens of DNA damaging agents cisplatin screen
cacccguacaucaaugucutt

SEQ ID NO: 108 - DNA, 21 bp, Artificial Sequence
  siRNA with 19 paired ribonucleotides and two unpaired nucleotides(“t”) on the 3′ end of each strand used in screens of DNA damaging agents cisplatin screen
ggaauaguaugcgcagcuutt

SEQ ID NO: 109 - DNA, 21 bp, Artificial Sequence
  siRNA with 19 paired ribonucleotides and two unpaired nucleotides(“t”) on the 3′ end of each strand used in screens of DNA damaging agents cisplatin screen
gugauucagauggagcuagtt

SEQ ID NO: 110 - DNA, 21 bp, Artificial Sequence
  siRNA with 19 paired ribonucleotides and two unpaired nucleotides(“t”) on the 3′ end of each strand used in screens of DNA damaging agents cisplatin screen
gaccgcaaacuacugauuctt

SEQ ID NO: 111 - DNA, 21 bp, Artificial Sequence
  siRNA with 19 paired ribonucleotides and two unpaired nucleotides(“t”) on the 3′ end of each strand used in screens of DNA damaging agents cisplatin screen
gccagcaugaucuccaagutt

SEQ ID NO: 112 - DNA, 21 bp, Artificial Sequence
  siRNA with 19 paired ribonucleotides and two unpaired nucleotides(“t”) on the 3′ end of each strand used in screens of DNA damaging agents cisplatin screen
uagacauuggguucgccgutt

SEQ ID NO: 113 - DNA, 21 bp, Artificial Sequence
  siRNA with 19 paired ribonucleotides and two unpaired nucleotides(“t”) on the 3′ end of each strand used in screens of DNA damaging agents cisplatin screen
ccucgagaaaaagggcugatt

SEQ ID NO: 114 - DNA, 21 bp, Artificial Sequence
  siRNA with 19 paired ribonucleotides and two unpaired nucleotides(“t”) on the 3′ end of each strand used in screens of DNA damaging agents cisplatin screen
gcucagcugaaagcuugcatt

SEQ ID NO: 115 - DNA, 21 bp, Artificial Sequence
  siRNA with 19 paired ribonucleotides and two unpaired nucleotides(“t”) on the 3′ end of each strand used in screens of DNA damaging agents cisplatin screen
ugccuagccgaguauucuutt

SEQ ID NO: 116 - DNA, 21 bp, Artificial Sequence
  siRNA with 19 paired ribonucleotides and two unpaired nucleotides(“t”) on the 3′ end of each strand used in screens of DNA damaging agents cisplatin screen
accuuauggaaaaggggugtt

SEQ ID NO: 117 - DNA, 21 bp, Artificial Sequence
  siRNA with 19 paired ribonucleotides and two unpaired nucleotides(“t”) on the 3′ end of each strand used in screens of DNA damaging agents cisplatin screen
ccauccuguuugaaagccutt

SEQ ID NO: 118 - DNA, 21 bp, Artificial Sequence
  siRNA with 19 paired ribonucleotides and two unpaired nucleotides(“t”) on the 3′ end of each strand used in screens of DNA damaging agents cisplatin screen
cccaaaaggaagugcuguatt

SEQ ID NO: 119 - DNA, 21 bp, Artificial Sequence
  siRNA with 19 paired ribonucleotides and two unpaired nucleotides(“t”) on the 3′ end of each strand used in screens of DNA damaging agents cisplatin screen
aggaugaugaugcaguucctt

SEQ ID NO: 120 - DNA, 21 bp, Artificial Sequence
  siRNA with 19 paired ribonucleotides and two unpaired nucleotides(“t”) on the 3′ end of each strand used in screens of DNA damaging agents cisplatin screen
gacaaggagaaugugcgcutt

SEQ ID NO: 121 - DNA, 21 bp, Artificial Sequence
  siRNA with 19 paired ribonucleotides and two unpaired nucleotides(“t”) on the 3′ end of each strand used in screens of DNA damaging agents cisplatin screen
gagcccagucuguugaguutt

SEQ ID NO: 122 - DNA, 21 bp, Artificial Sequence
  siRNA with 19 paired ribonucleotides and two unpaired nucleotides(“t”) on the 3′ end of each strand used in screens of DNA damaging agents cisplatin screen
acauucccuggaguguacatt

SEQ ID NO: 123 - DNA, 21 bp, Artificial Sequence
  siRNA with 19 paired ribonucleotides and two unpaired nucleotides(“t”) on the 3′ end of each strand used in screens of DNA damaging agents cisplatin screen
gaggauuuagcggcauuugtt

SEQ ID NO: 124 - DNA, 21 bp, Artificial Sequence
  siRNA with 19 paired ribonucleotides and two unpaired nucleotides(“t”) on the 3′ end of each strand used in screens of DNA damaging agents cisplatin screen
gcugcuggacugcauaaagtt

SEQ ID NO: 125 - DNA, 21 bp, Artificial Sequence
  siRNA with 19 paired ribonucleotides and two unpaired nucleotides(“t”) on the 3′ end of each strand used in screens of DNA damaging agents cisplatin screen
agagggaagacuugguggutt

SEQ ID NO: 126 - DNA, 21 bp, Artificial Sequence
  siRNA with 19 paired ribonucleotides and two unpaired nucleotides(“t”) on the 3′ end of each strand used in screens of DNA damaging agents cisplatin screen
cacucaugccaggacauugtt

SEQ ID NO: 127 - DNA, 21 bp, Artificial Sequence
  siRNA with 19 paired ribonucleotides and two unpaired nucleotides(“t”) on the 3′ end of each strand used in screens of DNA damaging agents cisplatin screen
gaagaaucgggacuacccatt

SEQ ID NO: 128 - DNA, 21 bp, Artificial Sequence
  siRNA with 19 paired ribonucleotides and two unpaired nucleotides(“t”) on the 3′ end of each strand used in screens of DNA damaging agents cisplatin screen
acucacagaaaaaccgucgtt

SEQ ID NO: 129 - DNA, 21 bp, Artificial Sequence
  siRNA with 19 paired ribonucleotides and two unpaired nucleotides(“t”) on the 3′ end of each strand used in screens of DNA damaging agents cisplatin screen
augaugggcggacgaguautt

SEQ ID NO: 130 - DNA, 21 bp, Artificial Sequence
  siRNA with 19 paired ribonucleotides and two unpaired nucleotides(“t”) on the 3′ end of each strand used in screens of DNA damaging agents cisplatin screen
gagucagcaccaucaaaugtt

SEQ ID NO: 131 - DNA, 21 bp, Artificial Sequence
  siRNA with 19 paired ribonucleotides and two unpaired nucleotides(“t”) on the 3′ end of each strand used in screens of DNA damaging agents cisplatin screen
agaggacguuuucuacggctt

SEQ ID NO: 132 - DNA, 21 bp, Artificial Sequence
  siRNA with 19 paired ribonucleotides and two unpaired nucleotides(“t”) on the 3′ end of each strand used in screens of DNA damaging agents cisplatin screen
aucuguuugcaaggggaagtt

SEQ ID NO: 133 - DNA, 21 bp, Artificial Sequence
  siRNA with 19 paired ribonucleotides and two unpaired nucleotides(“t”) on the 3′ end of each strand used in screens of DNA damaging agents cisplatin screen
caagaucauccccaagccatt

SEQ ID NO: 134 - DNA, 21 bp, Artificial Sequence
  siRNA with 19 paired ribonucleotides and two unpaired nucleotides(“t”) on the 3′ end of each strand used in screens of DNA damaging agents cisplatin screen
caccaagaaguucaucucctt

SEQ ID NO: 135 - DNA, 21 bp, Artificial Sequence
  siRNA with 19 paired ribonucleotides and two unpaired nucleotides(“t”) on the 3′ end of each strand used in screens of DNA damaging agents cisplatin screen
gagugucuggagcaaguugtt

SEQ ID NO: 136 - DNA, 21 bp, Artificial Sequence
  siRNA with 19 paired ribonucleotides and two unpaired nucleotides(“t”) on the 3′ end of each strand used in screens of DNA damaging agents cisplatin screen
guuuggaagaaccccacautt

SEQ ID NO: 137 - DNA, 21 bp, Artificial Sequence
  siRNA with 19 paired ribonucleotides and two unpaired nucleotides(“t”) on the 3′ end of each strand used in screens of DNA damaging agents cisplatin screen
acacuugguagacgggacutt

SEQ ID NO: 138 - DNA, 21 bp, Artificial Sequence
  siRNA with 19 paired ribonucleotides and two unpaired nucleotides(“t”) on the 3′ end of each strand used in screens of DNA damaging agents cisplatin screen
gucaaucauccacagagactt

SEQ ID NO: 139 - DNA, 21 bp, Artificial Sequence
  siRNA with 19 paired ribonucleotides and two unpaired nucleotides(“t”) on the 3′ end of each strand used in screens of DNA damaging agents cisplatin screen
uugcauguggaaguguuggtt

SEQ ID NO: 140 - DNA, 21 bp, Artificial Sequence
  siRNA with 19 paired ribonucleotides and two unpaired nucleotides(“t”) on the 3′ end of each strand used in screens of DNA damaging agents cisplatin screen
gaguacucuauaguggccutt

SEQ ID NO: 141 - DNA, 21 bp, Artificial Sequence
  siRNA with 19 paired ribonucleotides and two unpaired nucleotides(“t”) on the 3′ end of each strand used in screens of DNA damaging agents cisplatin screen
gcuucccaguccaaaugactt

SEQ ID NO: 142 - DNA, 21 bp, Artificial Sequence
  siRNA with 19 paired ribonucleotides and two unpaired nucleotides(“t”) on the 3′ end of each strand used in screens of DNA damaging agents cisplatin screen
ugacaguggagcauguguutt

SEQ ID NO: 143 - DNA, 21 bp, Artificial Sequence
  siRNA with 19 paired ribonucleotides and two unpaired nucleotides(“t”) on the 3′ end of each strand used in screens of DNA damaging agents cisplatin screen
aucagugauguggugcagatt

SEQ ID NO: 144 - DNA, 21 bp, Artificial Sequence
  siRNA with 19 paired ribonucleotides and two unpaired nucleotides(“t”) on the 3′ end of each strand used in screens of DNA damaging agents cisplatin screen
gacucggacacugaagaaatt

SEQ ID NO: 145 - DNA, 21 bp, Artificial Sequence
  siRNA with 19 paired ribonucleotides and two unpaired nucleotides(“t”) on the 3′ end of each strand used in screens of DNA damaging agents cisplatin screen
uggcacagcagguacuaaatt

SEQ ID NO: 146 - DNA, 21 bp, Artificial Sequence
  siRNA with 19 paired ribonucleotides and two unpaired nucleotides(“t”) on the 3′ end of each strand used in screens of DNA damaging agents cisplatin screen
gaaaagacuccuggcugugtt

SEQ ID NO: 147 - DNA, 21 bp, Artificial Sequence
  siRNA with 19 paired ribonucleotides and two unpaired nucleotides(“t”) on the 3′ end of each strand used in screens of DNA damaging agents cisplatin screen
ggacucugaagauguaccutt

SEQ ID NO: 148 - DNA, 21 bp, Artificial Sequence
  siRNA with 19 paired ribonucleotides and two unpaired nucleotides(“t”) on the 3′ end of each strand used in screens of DNA damaging agents cisplatin screen
ggcauacuaguacaaguggtt

SEQ ID NO: 149 - DNA, 21 bp, Artificial Sequence
  siRNA with 19 paired ribonucleotides and two unpaired nucleotides(“t”) on the 3′ end of each strand used in screens of DNA damaging agents cisplatin screen
auccuggagaugaccgugatt

SEQ ID NO: 150 - DNA, 21 bp, Artificial Sequence
  siRNA with 19 paired ribonucleotides and two unpaired nucleotides(“t”) on the 3′ end of each strand used in screens of DNA damaging agents cisplatin screen
gccggucauggagaagcggtt

SEQ ID NO: 151 - DNA, 21 bp, Artificial Sequence
  siRNA with 19 paired ribonucleotides and two unpaired nucleotides(“t”) on the 3′ end of each strand used in screens of DNA damaging agents cisplatin screen
uggcccugagacugcaucgtt

SEQ ID NO: 152 - DNA, 21 bp, Artificial Sequence
  siRNA with 19 paired ribonucleotides and two unpaired nucleotides(“t”) on the 3′ end of each strand used in screens of DNA damaging agents cisplatin screen
gccauuccuuugucugccatt

SEQ ID NO: 153 - DNA, 21 bp, Artificial Sequence
  siRNA with 19 paired ribonucleotides and two unpaired nucleotides(“t”) on the 3′ end of each strand used in screens of DNA damaging agents cisplatin screen
gugaaaguagaagggcccatt

SEQ ID NO: 154 - DNA, 21 bp, Artificial Sequence
  siRNA with 19 paired ribonucleotides and two unpaired nucleotides(“t”) on the 3′ end of each strand used in screens of DNA damaging agents cisplatin screen
uucaagcugguggaugcagtt

SEQ ID NO: 155 - DNA, 21 bp, Artificial Sequence
  siRNA with 19 paired ribonucleotides and two unpaired nucleotides(“t”) on the 3′ end of each strand used in screens of DNA damaging agents cisplatin screen
aguaccaggauucuucagctt

SEQ ID NO: 156 - DNA, 21 bp, Artificial Sequence
  siRNA with 19 paired ribonucleotides and two unpaired nucleotides(“t”) on the 3′ end of each strand used in screens of DNA damaging agents cisplatin screen
cuuaguucugggccauguatt

SEQ ID NO: 157 - DNA, 21 bp, Artificial Sequence
  siRNA with 19 paired ribonucleotides and two unpaired nucleotides(“t”) on the 3′ end of each strand used in screens of DNA damaging agents cisplatin screen
gaccccacugacagugauutt

SEQ ID NO: 158 - DNA, 21 bp, Artificial Sequence
  siRNA with 19 paired ribonucleotides and two unpaired nucleotides(“t”) on the 3′ end of each strand used in screens of DNA damaging agents cisplatin screen
agcuacaccacagaugccatt

SEQ ID NO: 159 - DNA, 21 bp, Artificial Sequence
  siRNA with 19 paired ribonucleotides and two unpaired nucleotides(“t”) on the 3′ end of each strand used in screens of DNA damaging agents cisplatin screen
ggacuuugcagcugacucutt

SEQ ID NO: 160 - DNA, 21 bp, Artificial Sequence
  siRNA with 19 paired ribonucleotides and two unpaired nucleotides(“t”) on the 3′ end of each strand used in screens of DNA damaging agents cisplatin screen
uaaagguugggguggccautt

SEQ ID NO: 161 - DNA, 21 bp, Artificial Sequence
  siRNA with 19 paired ribonucleotides and two unpaired nucleotides(“t”) on the 3′ end of each strand used in screens of DNA damaging agents cisplatin screen
aagcuaaugacgaggaacctt

SEQ ID NO: 162 - DNA, 21 bp, Artificial Sequence
  siRNA with 19 paired ribonucleotides and two unpaired nucleotides(“t”) on the 3′ end of each strand used in screens of DNA damaging agents cisplatin screen
ccauggugaagugcuuggatt

SEQ ID NO: 163 - DNA, 21 bp, Artificial Sequence
  siRNA with 19 paired ribonucleotides and two unpaired nucleotides(“t”) on the 3′ end of each strand used in screens of DNA damaging agents cisplatin screen
uaacagcugcaauucccugtt

SEQ ID NO: 164 - DNA, 21 bp, Artificial Sequence
  siRNA with 19 paired ribonucleotides and two unpaired nucleotides(“t”) on the 3′ end of each strand used in screens of DNA damaging agents cisplatin screen
ccugaugaaugcuguuguctt

SEQ ID NO: 165 - DNA, 21 bp, Artificial Sequence
  siRNA with 19 paired ribonucleotides and two unpaired nucleotides(“t”) on the 3′ end of each strand used in screens of DNA damaging agents cisplatin screen
gcacaauacggugaccaautt

SEQ ID NO: 166 - DNA, 21 bp, Artificial Sequence
  siRNA with 19 paired ribonucleotides and two unpaired nucleotides(“t”) on the 3′ end of each strand used in screens of DNA damaging agents cisplatin screen
ucacaucuuggaggaugugtt

SEQ ID NO: 167 - DNA, 21 bp, Artificial Sequence
  siRNA with 19 paired ribonucleotides and two unpaired nucleotides(“t”) on the 3′ end of each strand used in screens of DNA damaging agents cisplatin screen
gaaagugagcgacgaguuutt

SEQ ID NO: 168 - DNA, 21 bp, Artificial Sequence
  siRNA with 19 paired ribonucleotides and two unpaired nucleotides(“t”) on the 3′ end of each strand used in screens of DNA damaging agents cisplatin screen
gugccuucaacacagcuugtt

SEQ ID NO: 169 - DNA, 21 bp, Artificial Sequence
  siRNA with 19 paired ribonucleotides and two unpaired nucleotides(“t”) on the 3′ end of each strand used in screens of DNA damaging agents cisplatin screen
ugaauauccaagagcagggtt

SEQ ID NO: 170 - DNA, 21 bp, Artificial Sequence
  siRNA with 19 paired ribonucleotides and two unpaired nucleotides(“t”) on the 3′ end of each strand used in screens of DNA damaging agents cisplatin screen
gaagauccugagcaguauctt

SEQ ID NO: 171 - DNA, 21 bp, Artificial Sequence
  siRNA with 19 paired ribonucleotides and two unpaired nucleotides(“t”) on the 3′ end of each strand used in screens of DNA damaging agents cisplatin screen
gcugcagcaguacauugcutt

SEQ ID NO: 172 - DNA, 21 bp, Artificial Sequence
  siRNA with 19 paired ribonucleotides and two unpaired nucleotides(“t”) on the 3′ end of each strand used in screens of DNA damaging agents cisplatin screen
uacccuggacaagcucauctt

SEQ ID NO: 173 - DNA, 21 bp, Artificial Sequence
  siRNA with 19 paired ribonucleotides and two unpaired nucleotides(“t”) on the 3′ end of each strand used in screens of DNA damaging agents cisplatin screen
aacaagaggacacaauggctt

SEQ ID NO: 174 - DNA, 21 bp, Artificial Sequence
  siRNA with 19 paired ribonucleotides and two unpaired nucleotides(“t”) on the 3′ end of each strand used in screens of DNA damaging agents cisplatin screen
agaggugaagaucaugucgtt

SEQ ID NO: 175 - DNA, 21 bp, Artificial Sequence
  siRNA with 19 paired ribonucleotides and two unpaired nucleotides(“t”) on the 3′ end of each strand used in screens of DNA damaging agents cisplatin screen
ucgcagacuuuggcaugagtt

SEQ ID NO: 176 - DNA, 21 bp, Artificial Sequence
  siRNA with 19 paired ribonucleotides and two unpaired nucleotides(“t”) on the 3′ end of each strand used in screens of DNA damaging agents cisplatin screen
aucguuagcaccuuaggagtt

SEQ ID NO: 177 - DNA, 21 bp, Artificial Sequence
  siRNA with 19 paired ribonucleotides and two unpaired nucleotides(“t”) on the 3′ end of each strand used in screens of DNA damaging agents cisplatin screen
ccccugccuuguacauaautt

SEQ ID NO: 178 - DNA, 21 bp, Artificial Sequence
  siRNA with 19 paired ribonucleotides and two unpaired nucleotides(“t”) on the 3′ end of each strand used in screens of DNA damaging agents cisplatin screen
guacaaggaagcagcucgatt

SEQ ID NO: 179 - DNA, 21 bp, Artificial Sequence
  siRNA with 19 paired ribonucleotides and two unpaired nucleotides(“t”) on the 3′ end of each strand used in screens of DNA damaging agents cisplatin screen
agucccaggugggacucuutt

SEQ ID NO: 180 - DNA, 21 bp, Artificial Sequence
  siRNA with 19 paired ribonucleotides and two unpaired nucleotides(“t”) on the 3′ end of each strand used in screens of DNA damaging agents cisplatin screen
cuggcauccuugagcugactt

SEQ ID NO: 181 - DNA, 21 bp, Artificial Sequence
  siRNA with 19 paired ribonucleotides and two unpaired nucleotides(“t”) on the 3′ end of each strand used in screens of DNA damaging agents cisplatin screen
gacuguucuggaaugagggtt

SEQ ID NO: 182 - DNA, 21 bp, Artificial Sequence
  siRNA with 19 paired ribonucleotides and two unpaired nucleotides(“t”) on the 3′ end of each strand used in screens of DNA damaging agents cisplatin screen
acugccaggaguaagaacutt

SEQ ID NO: 183 - DNA, 21 bp, Artificial Sequence
  siRNA with 19 paired ribonucleotides and two unpaired nucleotides(“t”) on the 3′ end of each strand used in screens of DNA damaging agents cisplatin screen
cuauuacugcagagggcuutt

SEQ ID NO: 184 - DNA, 21 bp, Artificial Sequence
  siRNA with 19 paired ribonucleotides and two unpaired nucleotides(“t”) on the 3′ end of each strand used in screens of DNA damaging agents cisplatin screen
ugcauccugcagaguaucutt

SEQ ID NO: 185 - DNA, 21 bp, Artificial Sequence
  siRNA with 19 paired ribonucleotides and two unpaired nucleotides(“t”) on the 3′ end of each strand used in screens of DNA damaging agents cisplatin screen
ccuccuuucaaaccugcuutt

SEQ ID NO: 186 - DNA, 21 bp, Artificial Sequence
  siRNA with 19 paired ribonucleotides and two unpaired nucleotides(“t”) on the 3′ end of each strand used in screens of DNA damaging agents cisplatin screen
gagguucuguuuacagaggtt

SEQ ID NO: 187 - DNA, 21 bp, Artificial Sequence
  siRNA with 19 paired ribonucleotides and two unpaired nucleotides(“t”) on the 3′ end of each strand used in screens of DNA damaging agents cisplatin screen
ucagccagugcagauucaatt

SEQ ID NO: 188 - DNA, 21 bp, Artificial Sequence
  siRNA with 19 paired ribonucleotides and two unpaired nucleotides(“t”) on the 3′ end of each strand used in screens of DNA damaging agents cisplatin screen
agacaaagaggggaccuuctt

SEQ ID NO: 189 - DNA, 21 bp, Artificial Sequence
  siRNA with 19 paired ribonucleotides and two unpaired nucleotides(“t”) on the 3′ end of each strand used in screens of DNA damaging agents cisplatin screen
gaaagucuauccgaaggcutt

SEQ ID NO: 190 - DNA, 21 bp, Artificial Sequence
  siRNA with 19 paired ribonucleotides and two unpaired nucleotides(“t”) on the 3′ end of each strand used in screens of DNA damaging agents cisplatin screen
ugccucccugaaacuucgatt

SEQ ID NO: 191 - DNA, 21 bp, Artificial Sequence
  siRNA with 19 paired ribonucleotides and two unpaired nucleotides(“t”) on the 3′ end of each strand used in screens of DNA damaging agents cisplatin screen
aagcaagaaauggcggcagtt

SEQ ID NO: 192 - DNA, 21 bp, Artificial Sequence
  siRNA with 19 paired ribonucleotides and two unpaired nucleotides(“t”) on the 3′ end of each strand used in screens of DNA damaging agents cisplatin screen
gagcugaaugucuuugggctt

SEQ ID NO: 193 - DNA, 21 bp, Artificial Sequence
  siRNA with 19 paired ribonucleotides and two unpaired nucleotides(“t”) on the 3′ end of each strand used in screens of DNA damaging agents cisplatin screen
uguauauagcgaccagagctt

SEQ ID NO: 194 - DNA, 21 bp, Artificial Sequence
  siRNA with 19 paired ribonucleotides and two unpaired nucleotides(“t”) on the 3′ end of each strand used in screens of DNA damaging agents cisplatin screen
cuucagugcuggugaacuctt

SEQ ID NO: 195 - DNA, 21 bp, Artificial Sequence
  siRNA with 19 paired ribonucleotides and two unpaired nucleotides(“t”) on the 3′ end of each strand used in screens of DNA damaging agents cisplatin screen
gcuggaccacugcaauauutt

SEQ ID NO: 196 - DNA, 21 bp, Artificial Sequence
  siRNA with 19 paired ribonucleotides and two unpaired nucleotides(“t”) on the 3′ end of each strand used in screens of DNA damaging agents cisplatin screen
guggcuuacacggacaucatt

SEQ ID NO: 197 - DNA, 21 bp, Artificial Sequence
  siRNA with 19 paired ribonucleotides and two unpaired nucleotides(“t”) on the 3′ end of each strand used in screens of DNA damaging agents cisplatin screen
aacaggaccguacugcuugtt

SEQ ID NO: 198 - DNA, 21 bp, Artificial Sequence
  siRNA with 19 paired ribonucleotides and two unpaired nucleotides(“t”) on the 3′ end of each strand used in screens of DNA damaging agents cisplatin screen
gaagccuuuguucaggucutt

SEQ ID NO: 199 - DNA, 21 bp, Artificial Sequence
  siRNA with 19 paired ribonucleotides and two unpaired nucleotides(“t”) on the 3′ end of each strand used in screens of DNA damaging agents cisplatin screen
gagaggcauccuccuugaatt

SEQ ID NO: 200 - DNA, 21 bp, Artificial Sequence
  siRNA with 19 paired ribonucleotides and two unpaired nucleotides(“t”) on the 3′ end of each strand used in screens of DNA damaging agents cisplatin screen
ccagcacugacuacugugutt

SEQ ID NO: 201 - DNA, 21 bp, Artificial Sequence
  siRNA with 19 paired ribonucleotides and two unpaired nucleotides(“t”) on the 3′ end of each strand used in screens of DNA damaging agents cisplatin screen
ggaacucgaugcuugucagtt

SEQ ID NO: 202 - DNA, 21 bp, Artificial Sequence
  siRNA with 19 paired ribonucleotides and two unpaired nucleotides(“t”) on the 3′ end of each strand used in screens of DNA damaging agents cisplatin screen
ugcgaggaagauacggagutt

SEQ ID NO: 203 - DNA, 21 bp, Artificial Sequence
  siRNA with 19 paired ribonucleotides and two unpaired nucleotides(“t”) on the 3′ end of each strand used in screens of DNA damaging agents cisplatin screen
gcagaugagcaaggaugcutt

SEQ ID NO: 204 - DNA, 21 bp, Artificial Sequence
  siRNA with 19 paired ribonucleotides and two unpaired nucleotides(“t”) on the 3′ end of each strand used in screens of DNA damaging agents cisplatin screen
guacauccauguggccaaatt

SEQ ID NO: 205 - DNA, 21 bp, Artificial Sequence
  siRNA with 19 paired ribonucleotides and two unpaired nucleotides(“t”) on the 3′ end of each strand used in screens of DNA damaging agents cisplatin screen
ugggucaugaaagcugccatt

SEQ ID NO: 206 - DNA, 21 bp, Artificial Sequence
  siRNA with 19 paired ribonucleotides and two unpaired nucleotides(“t”) on the 3′ end of each strand used in screens of DNA damaging agents cisplatin screen
agaaccucggcuacaacgutt

SEQ ID NO: 207 - DNA, 21 bp, Artificial Sequence
  siRNA with 19 paired ribonucleotides and two unpaired nucleotides(“t”) on the 3′ end of each strand used in screens of DNA damaging agents cisplatin screen
uccgcaucuccaugugccatt

SEQ ID NO: 208 - DNA, 21 bp, Artificial Sequence
  siRNA with 19 paired ribonucleotides and two unpaired nucleotides(“t”) on the 3′ end of each strand used in screens of DNA damaging agents cisplatin screen
ucggcuacaacgugaccaatt

SEQ ID NO: 209 - DNA, 21 bp, Artificial Sequence
  siRNA with 19 paired ribonucleotides and two unpaired nucleotides(“t”) on the 3′ end of each strand used in screens of DNA damaging agents doxorubicin screen
cgcaagaagaaggccgugutt

SEQ ID NO: 210 - DNA, 21 bp, Artificial Sequence
  siRNA with 19 paired ribonucleotides and two unpaired nucleotides(“t”) on the 3′ end of each strand used in screens of DNA damaging agents doxorubicin screen
cgcuggugcaauguuuucutt

SEQ ID NO: 211 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaaucccuaccgagacucutt

SEQ ID NO: 212 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaaccucuacacguucugctt

SEQ ID NO: 213 - DNA, 21 bp, Artificial Sequence
  siRNA with 19 paired ribonucleotides and two unpaired nucleotides(“t”) on the 3′ end of each strand used in screens of DNA damaging agents doxorubicin screen
cuaaaggugaaaagcuccgtt

SEQ ID NO: 214 - DNA, 21 bp, Artificial Sequence
  siRNA with 19 paired ribonucleotides and two unpaired nucleotides(“t”) on the 3′ end of each strand used in screens of DNA damaging agents doxorubicin screen
uccuggcaagaaagcuugatt

SEQ ID NO: 215 - DNA, 21 bp, Artificial Sequence
  siRNA with 19 paired ribonucleotides and two unpaired nucleotides(“t”) on the 3′ end of each strand used in screens of DNA damaging agents doxorubicin screen
aagauggccacaaaccugctt

SEQ ID NO: 216 - DNA, 21 bp, Artificial Sequence
  siRNA with 19 paired ribonucleotides and two unpaired nucleotides(“t”) on the 3′ end of each strand used in screens of DNA damaging agents doxorubicin screen
agauaaacggcggcauugutt

SEQ ID NO: 217 - DNA, 21 bp, Artificial Sequence
  siRNA with 19 paired ribonucleotides and two unpaired nucleotides(“t”) on the 3′ end of each strand used in screens of DNA damaging agents doxorubicin screen
gacaugcaggaaguuguugtt

SEQ ID NO: 218 - DNA, 21 bp, Artificial Sequence
  siRNA with 19 paired ribonucleotides and two unpaired nucleotides(“t”) on the 3′ end of each strand used in screens of DNA damaging agents doxorubicin screen
cgggagaucuucagcacactt

SEQ ID NO: 219 - DNA, 21 bp, Artificial Sequence
  siRNA with 19 paired ribonucleotides and two unpaired nucleotides(“t”) on the 3′ end of each strand used in screens of DNA damaging agents doxorubicin screen
gagauuggccagcacccuutt

SEQ ID NO: 220 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcccaggacaugagugucutt

SEQ ID NO: 221 - DNA, 21 bp, Artificial Sequence
  siRNA with 19 paired ribonucleotides and two unpaired nucleotides(“t”) on the 3′ end of each strand used in screens of DNA damaging agents doxorubicin screen
cgaggaugaggcaucugautt

SEQ ID NO: 222 - DNA, 21 bp, Artificial Sequence
  siRNA with 19 paired ribonucleotides and two unpaired nucleotides(“t”) on the 3′ end of each strand used in screens of DNA damaging agents doxorubicin screen
cugaagucccuuuuggaggtt

SEQ ID NO: 223 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaacuucccuuuggucauctt

SEQ ID NO: 224 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcuggagaaccucaugcugtt

SEQ ID NO: 225 - DNA, 21 bp, Artificial Sequence
  siRNA with 19 paired ribonucleotides and two unpaired nucleotides(“t”) on the 3′ end of each strand used in screens of DNA damaging agents doxorubicin screen
agacguuuuugugcuguggtt

SEQ ID NO: 226 - DNA, 21 bp, Artificial Sequence
  siRNA with 19 paired ribonucleotides and two unpaired nucleotides(“t”) on the 3′ end of each strand used in screens of DNA damaging agents doxorubicin screen
cgcaccuuccauguggagatt

SEQ ID NO: 227 - DNA, 21 bp, Artificial Sequence
  siRNA with 19 paired ribonucleotides and two unpaired nucleotides(“t”) on the 3′ end of each strand used in screens of DNA damaging agents doxorubicin screen
agauggccacaucaagauctt

SEQ ID NO: 228 - DNA, 21 bp, Artificial Sequence
  siRNA with 19 paired ribonucleotides and two unpaired nucleotides(“t”) on the 3′ end of each strand used in screens of DNA damaging agents doxorubicin screen
gucaucauugccaaggaugtt

SEQ ID NO: 229 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugccagcugaugaagaccgtt

SEQ ID NO: 230 - DNA, 21 bp, Artificial Sequence
  siRNA with 19 paired ribonucleotides and two unpaired nucleotides(“t”) on the 3′ end of each strand used in screens of DNA damaging agents doxorubicin screen
accagagaccaaaugucactt

SEQ ID NO: 231 - DNA, 21 bp, Artificial Sequence
  siRNA with 19 paired ribonucleotides and two unpaired nucleotides(“t”) on the 3′ end of each strand used in screens of DNA damaging agents doxorubicin screen
auaagcccaccagcuugugtt

SEQ ID NO: 232 - DNA, 21 bp, Artificial Sequence
  siRNA with 19 paired ribonucleotides and two unpaired nucleotides(“t”) on the 3′ end of each strand used in screens of DNA damaging agents doxorubicin screen
ucaacaccgcuuugccgautt

SEQ ID NO: 233 - DNA, 21 bp, Artificial Sequence
  siRNA with 19 paired ribonucleotides and two unpaired nucleotides(“t”) on the 3′ end of each strand used in screens of DNA damaging agents doxorubicin screen
cuggcugauuuuggucuugtt

SEQ ID NO: 234 - DNA, 21 bp, Artificial Sequence
  siRNA with 19 paired ribonucleotides and two unpaired nucleotides(“t”) on the 3′ end of each strand used in screens of DNA damaging agents doxorubicin screen
gccuucauguugucuggaatt

SEQ ID NO: 235 - DNA, 21 bp, Artificial Sequence
  siRNA with 19 paired ribonucleotides and two unpaired nucleotides(“t”) on the 3′ end of each strand used in screens of DNA damaging agents doxorubicin screen
uccacaccaaagagacacutt

SEQ ID NO: 236 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaaagugagcgacgaguuutt

SEQ ID NO: 237 - DNA, 21 bp, Artificial Sequence
  siRNA with 19 paired ribonucleotides and two unpaired nucleotides(“t”) on the 3′ end of each strand used in screens of DNA damaging agents doxorubicin screen
gugccuucaacacagcuugtt

SEQ ID NO: 238 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugaauauccaagagcagggtt

SEQ ID NO: 239 - DNA, 21 bp, Artificial Sequence
  siRNA with 19 paired ribonucleotides and two unpaired nucleotides(“t”) on the 3′ end of each strand used in screens of DNA damaging agents doxorubicin screen
agucacgagcguucgaaaatt

SEQ ID NO: 240 - DNA, 21 bp, Artificial Sequence
  siRNA with 19 paired ribonucleotides and two unpaired nucleotides(“t”) on the 3′ end of each strand used in screens of DNA damaging agents doxorubicin screen
caacaugaagguggccauutt

SEQ ID NO: 241 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcacuauaagauccgcugctt

SEQ ID NO: 242 - DNA, 21 bp, Artificial Sequence
  siRNA with 19 paired ribonucleotides and two unpaired nucleotides(“t”) on the 3′ end of each strand used in screens of DNA damaging agents doxorubicin screen
caaaucugugagcccaacatt

SEQ ID NO: 243 - DNA, 21 bp, Artificial Sequence
  siRNA with 19 paired ribonucleotides and two unpaired nucleotides(“t”) on the 3′ end of each strand used in screens of DNA damaging agents doxorubicin screen
caagauguucuugcacaggtt

SEQ ID NO: 244 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaacggcuaugugcguuuatt

SEQ ID NO: 245 - DNA, 21 bp, Artificial Sequence
  siRNA with 19 paired ribonucleotides and two unpaired nucleotides(“t”) on the 3′ end of each strand used in screens of DNA damaging agents doxorubicin screen
acacuugguagacgggacutt

SEQ ID NO: 246 - DNA, 21 bp, Artificial Sequence
  siRNA with 19 paired ribonucleotides and two unpaired nucleotides(“t”) on the 3′ end of each strand used in screens of DNA damaging agents doxorubicin screen
gucaaucauccacagagactt

SEQ ID NO: 247 - DNA, 21 bp, Artificial Sequence
  siRNA with 19 paired ribonucleotides and two unpaired nucleotides(“t”) on the 3′ end of each strand used in screens of DNA damaging agents doxorubicin screen
uugcauguggaaguguuggtt

SEQ ID NO: 248 - DNA, 21 bp, Artificial Sequence
  siRNA with 19 paired ribonucleotides and two unpaired nucleotides(“t”) on the 3′ end of each strand used in screens of DNA damaging agents doxorubicin screen
agucccaggugggacucuutt

SEQ ID NO: 249 - DNA, 21 bp, Artificial Sequence
  siRNA with 19 paired ribonucleotides and two unpaired nucleotides(“t”) on the 3′ end of each strand used in screens of DNA damaging agents doxorubicin screen
cuggcauccuugagcugactt

SEQ ID NO: 250 - DNA, 21 bp, Artificial Sequence
  siRNA with 19 paired ribonucleotides and two unpaired nucleotides(“t”) on the 3′ end of each strand used in screens of DNA damaging agents doxorubicin screen
gacuguucuggaaugagggtt

SEQ ID NO: 251 - DNA, 21 bp, Artificial Sequence
  siRNA with 19 paired ribonucleotides and two unpaired nucleotides(“t”) on the 3′ end of each strand used in screens of DNA damaging agents doxorubicin screen
gacauuguggccagagagutt

SEQ ID NO: 252 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaugaggaccucaaagugctt

SEQ ID NO: 253 - DNA, 21 bp, Artificial Sequence
  siRNA with 19 paired ribonucleotides and two unpaired nucleotides(“t”) on the 3′ end of each strand used in screens of DNA damaging agents doxorubicin screen
ggcuggagccuaugauuuctt

SEQ ID NO: 254 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaagcaugcccagaccuuutt

SEQ ID NO: 255 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaggaucuuuguggccaagtt

SEQ ID NO: 256 - DNA, 21 bp, Artificial Sequence
  siRNA with 19 paired ribonucleotides and two unpaired nucleotides(“t”) on the 3′ end of each strand used in screens of DNA damaging agents doxorubicin screen
cuaccuggaucgcuaccugtt

SEQ ID NO: 257 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccacagaugagguccauactt

SEQ ID NO: 258 - DNA, 21 bp, Artificial Sequence
  siRNA with 19 paired ribonucleotides and two unpaired nucleotides(“t”) on the 3′ end of each strand used in screens of DNA damaging agents doxorubicin screen
cuggggcuuucuugacaugtt

SEQ ID NO: 259 - DNA, 21 bp, Artificial Sequence
  siRNA with 19 paired ribonucleotides and two unpaired nucleotides(“t”) on the 3′ end of each strand used in screens of DNA damaging agents doxorubicin screen
gugguuaagaaagccucagtt

SEQ ID NO: 260 - DNA, 21 bp, Artificial Sequence
  siRNA with 19 paired ribonucleotides and two unpaired nucleotides(“t”) on the 3′ end of each strand used in screens of DNA damaging agents doxorubicin screen
auggauuguuucugggcgutt

SEQ ID NO: 261 - DNA, 21 bp, Artificial Sequence
  siRNA with 19 paired ribonucleotides and two unpaired nucleotides(“t”) on the 3′ end of each strand used in screens of DNA damaging agents doxorubicin screen
cuaucagucuucccacagctt

SEQ ID NO: 262 - DNA, 21 bp, Artificial Sequence
  siRNA with 19 paired ribonucleotides and two unpaired nucleotides(“t”) on the 3′ end of each strand used in screens of DNA damaging agents doxorubicin screen
cuugccacuugaaaggagatt

SEQ ID NO: 263 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccucgagaaaaagggcugatt

SEQ ID NO: 264 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcucagcugaaagcuugcatt

SEQ ID NO: 265 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugccuagccgaguauucuutt

SEQ ID NO: 266 - DNA, 21 bp, Artificial Sequence
  siRNA with 19 paired ribonucleotides and two unpaired nucleotides(“t”) on the 3′ end of each strand used in screens of DNA damaging agents doxorubicin screen
gacccgcuaucccauauugtt

SEQ ID NO: 267 - DNA, 21 bp, Artificial Sequence
  siRNA with 19 paired ribonucleotides and two unpaired nucleotides(“t”) on the 3′ end of each strand used in screens of DNA damaging agents doxorubicin screen
gccagcaaugccaagaucutt

SEQ ID NO: 268 - DNA, 21 bp, Artificial Sequence
  siRNA with 19 paired ribonucleotides and two unpaired nucleotides(“t”) on the 3′ end of each strand used in screens of DNA damaging agents doxorubicin screen
uugcccugacugccauuuutt

SEQ ID NO: 269 - DNA, 21 bp, Artificial Sequence
  siRNA with 19 paired ribonucleotides and two unpaired nucleotides(“t”) on the 3′ end of each strand used in screens of DNA damaging agents doxorubicin screen
agcgccaguaaagaagaactt

SEQ ID NO: 270 - DNA, 21 bp, Artificial Sequence
  siRNA with 19 paired ribonucleotides and two unpaired nucleotides(“t”) on the 3′ end of each strand used in screens of DNA damaging agents doxorubicin screen
agggcagccaguugucauutt

SEQ ID NO: 271 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccaccacuccaaaaugagctt

SEQ ID NO: 272 - DNA, 21 bp, Artificial Sequence
  siRNA with 19 paired ribonucleotides and two unpaired nucleotides(“t”) on the 3′ end of each strand used in screens of DNA damaging agents doxorubicin screen
aggaugaugaugcaguucctt

SEQ ID NO: 273 - DNA, 21 bp, Artificial Sequence
  siRNA with 19 paired ribonucleotides and two unpaired nucleotides(“t”) on the 3′ end of each strand used in screens of DNA damaging agents doxorubicin screen
gacaaggagaaugugcgcutt

SEQ ID NO: 274 - DNA, 21 bp, Artificial Sequence
  siRNA with 19 paired ribonucleotides and two unpaired nucleotides(“t”) on the 3′ end of each strand used in screens of DNA damaging agents doxorubicin screen
gagcccagucuguugaguutt

SEQ ID NO: 275 - DNA, 21 bp, Artificial Sequence
  siRNA with 19 paired ribonucleotides and two unpaired nucleotides(“t”) on the 3′ end of each strand used in screens of DNA damaging agents doxorubicin screen
accuuauggaaaaggggugtt

SEQ ID NO: 276 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccauccuguuugaaagccutt

SEQ ID NO: 277 - DNA, 21 bp, Artificial Sequence
  siRNA with 19 paired ribonucleotides and two unpaired nucleotides(“t”) on the 3′ end of each strand used in screens of DNA damaging agents doxorubicin screen
cccaaaaggaagugcuguatt

SEQ ID NO: 278 - DNA, 21 bp, Artificial Sequence
  siRNA with 19 paired ribonucleotides and two unpaired nucleotides(“t”) on the 3′ end of each strand used in screens of DNA damaging agents doxorubicin screen
cgagaggaagcucuaucugtt

SEQ ID NO: 279 - DNA, 21 bp, Artificial Sequence
  siRNA with 19 paired ribonucleotides and two unpaired nucleotides(“t”) on the 3′ end of each strand used in screens of DNA damaging agents doxorubicin screen
gagaggaugcaucuggggatt

SEQ ID NO: 280 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaucagacuggauuuggagtt

SEQ ID NO: 281 - DNA, 21 bp, Artificial Sequence
  siRNA with 19 paired ribonucleotides and two unpaired nucleotides(“t”) on the 3′ end of each strand used in screens of DNA damaging agents doxorubicin screen
cagucaagcuggcugacuutt

SEQ ID NO: 282 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcgaaucucugccuuucgatt

SEQ ID NO: 283 - DNA, 21 bp, Artificial Sequence
  siRNA with 19 paired ribonucleotides and two unpaired nucleotides(“t”) on the 3′ end of each strand used in screens of DNA damaging agents doxorubicin screen
ggaucugaugcgccaguuutt

SEQ ID NO: 284 - DNA, 21 bp, Artificial Sequence
  siRNA with 19 paired ribonucleotides and two unpaired nucleotides(“t”) on the 3′ end of each strand used in screens of DNA damaging agents doxorubicin screen
cccugguguuugagcaugutt

SEQ ID NO: 285 - DNA, 21 bp, Artificial Sequence
  siRNA with 19 paired ribonucleotides and two unpaired nucleotides(“t”) on the 3′ end of each strand used in screens of DNA damaging agents doxorubicin screen
cugaccgggagaucaaggutt

SEQ ID NO: 286 - DNA, 21 bp, Artificial Sequence
  siRNA with 19 paired ribonucleotides and two unpaired nucleotides(“t”) on the 3′ end of each strand used in screens of DNA damaging agents doxorubicin screen
gagugugagaguccccaautt

SEQ ID NO: 287 - DNA, 21 bp, Artificial Sequence
  siRNA with 19 paired ribonucleotides and two unpaired nucleotides(“t”) on the 3′ end of each strand used in screens of DNA damaging agents doxorubicin screen
aacuaggcgguugaaugagtt

SEQ ID NO: 288 - DNA, 21 bp, Artificial Sequence
  siRNA with 19 paired ribonucleotides and two unpaired nucleotides(“t”) on the 3′ end of each strand used in screens of DNA damaging agents doxorubicin screen
cauacuggccuggacuguutt

SEQ ID NO: 289 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaugguggcaguagaggcutt

SEQ ID NO: 290 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaaaaccgggauuccggcctt

SEQ ID NO: 291 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcgcaagagaucagcgccutt

SEQ ID NO: 292 - DNA, 21 bp, Artificial Sequence
  siRNA with 19 paired ribonucleotides and two unpaired nucleotides(“t”) on the 3′ end of each strand used in screens of DNA damaging agents doxorubicin screen
guggacagcgacucggugctt

SEQ ID NO: 293 - DNA, 21 bp, Artificial Sequence
  siRNA with 19 paired ribonucleotides and two unpaired nucleotides(“t”) on the 3′ end of each strand used in screens of DNA damaging agents doxorubicin screen
acacagagaagcggauuuctt

SEQ ID NO: 294 - DNA, 21 bp, Artificial Sequence
  siRNA with 19 paired ribonucleotides and two unpaired nucleotides(“t”) on the 3′ end of each strand used in screens of DNA damaging agents doxorubicin screen
cuccaagagguggguaauutt

SEQ ID NO: 295 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugucugcugaggaguuaugtt

SEQ ID NO: 296 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccugccuuaaaaauuaccgtt

SEQ ID NO: 297 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaacuaaagagcugugguatt

SEQ ID NO: 298 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaggauccggggcaauacatt

SEQ ID NO: 299 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaaaaugaagcuuugcgggtt

SEQ ID NO: 300 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaagagaucccagugcuuctt

SEQ ID NO: 301 - DNA, 21 bp, Artificial Sequence
  siRNA with 19 paired ribonucleotides and two unpaired nucleotides(“t”) on the 3′ end of each strand used in screens of DNA damaging agents doxorubicin screen
ucugaaagugaccagcucatt

SEQ ID NO: 302 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccaguugauguuugguccutt

SEQ ID NO: 303 - DNA, 21 bp, Artificial Sequence
  siRNA with 19 paired ribonucleotides and two unpaired nucleotides(“t”) on the 3′ end of each strand used in screens of DNA damaging agents doxorubicin screen
ucucagacuuuggcuuggctt

SEQ ID NO: 304 - DNA, 21 bp, Artificial Sequence
  siRNA with 19 paired ribonucleotides and two unpaired nucleotides(“t”) on the 3′ end of each strand used in screens of DNA damaging agents doxorubicin screen
uucuauggucacaggagagtt

SEQ ID NO: 305 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaaggcugcucacaaguuctt

SEQ ID NO: 306 - DNA, 21 bp, Artificial Sequence
  siRNA with 19 paired ribonucleotides and two unpaired nucleotides(“t”) on the 3′ end of each strand used in screens of DNA damaging agents doxorubicin screen
agcugcucaacaaaccagatt

SEQ ID NO: 307 - DNA, 21 bp, Artificial Sequence
  siRNA with 19 paired ribonucleotides and two unpaired nucleotides(“t”) on the 3′ end of each strand used in screens of DNA damaging agents doxorubicin screen
augaggaacagggcaauagtt

SEQ ID NO: 308 - DNA, 21 bp, Artificial Sequence
  siRNA with 19 paired ribonucleotides and two unpaired nucleotides(“t”) on the 3′ end of each strand used in screens of DNA damaging agents doxorubicin screen
gacaucccgagucuauaagtt

SEQ ID NO: 309 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcacaaggaggucuucuuctt

SEQ ID NO: 310 - DNA, 21 bp, Artificial Sequence
  siRNA with 19 paired ribonucleotides and two unpaired nucleotides(“t”) on the 3′ end of each strand used in screens of DNA damaging agents doxorubicin screen
uggaggagaauuaggccuutt

SEQ ID NO: 311 - DNA, 21 bp, Artificial Sequence
  siRNA with 19 paired ribonucleotides and two unpaired nucleotides(“t”) on the 3′ end of each strand used in screens of DNA damaging agents doxorubicin screen
cguuccgauccucuauacutt

SEQ ID NO: 312 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugacaucauugugcuggcctt

SEQ ID NO: 313 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugaccaaagaugaccugugtt

SEQ ID NO: 314 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccugaugaaugcuguuguctt

SEQ ID NO: 315 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcacaauacggugaccaautt

SEQ ID NO: 316 - DNA, 21 bp, Artificial Sequence
  siRNA with 19 paired ribonucleotides and two unpaired nucleotides(“t”) on the 3′ end of each strand used in screens of DNA damaging agents doxorubicin screen
ucacaucuuggaggaugugtt

SEQ ID NO: 317 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaguguuguugcuggcagatt

SEQ ID NO: 318 - DNA, 21 bp, Artificial Sequence
  siRNA with 19 paired ribonucleotides and two unpaired nucleotides(“t”) on the 3′ end of each strand used in screens of DNA damaging agents doxorubicin screen
acuugagaagcuuuuggggtt

SEQ ID NO: 319 - DNA, 21 bp, Artificial Sequence
  siRNA with 19 paired ribonucleotides and two unpaired nucleotides(“t”) on the 3′ end of each strand used in screens of DNA damaging agents doxorubicin screen
cuagagguuuuugcugcagtt

SEQ ID NO: 320 - DNA, 21 bp, Artificial Sequence
  siRNA with 19 paired ribonucleotides and two unpaired nucleotides(“t”) on the 3′ end of each strand used in screens of DNA damaging agents doxorubicin screen
aagagugagcuggucaaugtt

SEQ ID NO: 321 - DNA, 21 bp, Artificial Sequence
  siRNA with 19 paired ribonucleotides and two unpaired nucleotides(“t”) on the 3′ end of each strand used in screens of DNA damaging agents doxorubicin screen
auuuuggagugcuugggcatt

SEQ ID NO: 322 - DNA, 21 bp, Artificial Sequence
  siRNA with 19 paired ribonucleotides and two unpaired nucleotides(“t”) on the 3′ end of each strand used in screens of DNA damaging agents doxorubicin screen
uggguaaacagggcagcaatt

SEQ ID NO: 323 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaauauuuuugggacgccgtt

SEQ ID NO: 324 - DNA, 21 bp, Artificial Sequence
  siRNA with 19 paired ribonucleotides and two unpaired nucleotides(“t”) on the 3′ end of each strand used in screens of DNA damaging agents doxorubicin screen
uccaagaggcucucagacatt

SEQ ID NO: 325 - DNA, 21 bp, Artificial Sequence
  siRNA with 19 paired ribonucleotides and two unpaired nucleotides(“t”) on the 3′ end of each strand used in screens of DNA damaging agents doxorubicin screen
ucucagaagguccuccugatt

SEQ ID NO: 326 - DNA, 21 bp, Artificial Sequence
  siRNA with 19 paired ribonucleotides and two unpaired nucleotides(“t”) on the 3′ end of each strand used in screens of DNA damaging agents doxorubicin screen
ggaggagaucuuugaugactt

SEQ ID NO: 327 - DNA, 21 bp, Artificial Sequence
  siRNA with 19 paired ribonucleotides and two unpaired nucleotides(“t”) on the 3′ end of each strand used in screens of DNA damaging agents doxorubicin screen
guacgccagcagcuugcugtt

SEQ ID NO: 328 - DNA, 21 bp, Artificial Sequence
  siRNA with 19 paired ribonucleotides and two unpaired nucleotides(“t”) on the 3′ end of each strand used in screens of DNA damaging agents doxorubicin screen
ucggaucacacggcaccgatt

SEQ ID NO: 329 - DNA, 21 bp, Artificial Sequence
  siRNA with 19 paired ribonucleotides and two unpaired nucleotides(“t”) on the 3′ end of each strand used in screens of DNA damaging agents doxorubicin screen
accccagugaguucuuugutt

SEQ ID NO: 330 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccuggacaaugacacagagtt

SEQ ID NO: 331 - DNA, 21 bp, Artificial Sequence
  siRNA with 19 paired ribonucleotides and two unpaired nucleotides(“t”) on the 3′ end of each strand used in screens of DNA damaging agents doxorubicin screen
guucauuuaagccucagggtt

SEQ ID NO: 332 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccauguuugcauggccuuutt

SEQ ID NO: 333 - DNA, 21 bp, Artificial Sequence
  siRNA with 19 paired ribonucleotides and two unpaired nucleotides(“t”) on the 3′ end of each strand used in screens of DNA damaging agents doxorubicin screen
cuucuggagcaauccaaactt

SEQ ID NO: 334 - DNA, 21 bp, Artificial Sequence
  siRNA with 19 paired ribonucleotides and two unpaired nucleotides(“t”) on the 3′ end of each strand used in screens of DNA damaging agents doxorubicin screen
ucuuuggaugcccuccacatt

SEQ ID NO: 335 - DNA, 21 bp, Artificial Sequence
  siRNA with 19 paired ribonucleotides and two unpaired nucleotides(“t”) on the 3′ end of each strand used in screens of DNA damaging agents doxorubicin screen
acuggcuaaagaugcugugtt

SEQ ID NO: 336 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaccaugggaaaauuguggtt

SEQ ID NO: 337 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcuuaguacagcggguugatt

SEQ ID NO: 338 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccaccccuuccuuagguugtt

SEQ ID NO: 339 - DNA, 21 bp, Artificial Sequence
  siRNA with 19 paired ribonucleotides and two unpaired nucleotides(“t”) on the 3′ end of each strand used in screens of DNA damaging agents doxorubicin screen
cucaccugugaccaaaacatt

SEQ ID NO: 340 - DNA, 21 bp, Artificial Sequence
  siRNA with 19 paired ribonucleotides and two unpaired nucleotides(“t”) on the 3′ end of each strand used in screens of DNA damaging agents doxorubicin screen
uagcccagaagauaagcggtt

SEQ ID NO: 341 - DNA, 21 bp, Artificial Sequence
  siRNA with 19 paired ribonucleotides and two unpaired nucleotides(“t”) on the 3′ end of each strand used in screens of DNA damaging agents doxorubicin screen
gccauuccuuugucugccatt

SEQ ID NO: 342 - DNA, 21 bp, Artificial Sequence
  siRNA with 19 paired ribonucleotides and two unpaired nucleotides(“t”) on the 3′ end of each strand used in screens of DNA damaging agents doxorubicin screen
gugaaaguagaagggcccatt

SEQ ID NO: 343 - DNA, 21 bp, Artificial Sequence
  siRNA with 19 paired ribonucleotides and two unpaired nucleotides(“t”) on the 3′ end of each strand used in screens of DNA damaging agents doxorubicin screen
uucaagcugguggaugcagtt

SEQ ID NO: 344 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaagauccugagcaguauctt

SEQ ID NO: 345 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcugcagcaguacauugcutt

SEQ ID NO: 346 - DNA, 21 bp, Artificial Sequence
  siRNA with 19 paired ribonucleotides and two unpaired nucleotides(“t”) on the 3′ end of each strand used in screens of DNA damaging agents doxorubicin screen
uacccuggacaagcucauctt

SEQ ID NO: 347 - DNA, 21 bp, Artificial Sequence
  siRNA with 19 paired ribonucleotides and two unpaired nucleotides(“t”) on the 3′ end of each strand used in screens of DNA damaging agents doxorubicin screen
cuuucugaauggggagccutt

SEQ ID NO: 348 - DNA, 21 bp, Artificial Sequence
  siRNA with 19 paired ribonucleotides and two unpaired nucleotides(“t”) on the 3′ end of each strand used in screens of DNA damaging agents doxorubicin screen
uacacacaccagagugaugtt

SEQ ID NO: 349 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugacaguggagccuguguatt

SEQ ID NO: 350 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaguacucuauaguggccutt

SEQ ID NO: 351 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcuucccaguccaaaugactt

SEQ ID NO: 352 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugacaguggagcauguguutt

SEQ ID NO: 353 - DNA, 21 bp, Artificial Sequence
  siRNA with 19 paired ribonucleotides and two unpaired nucleotides(“t”) on the 3′ end of each strand used in screens of DNA damaging agents doxorubicin screen
aagccuuacggucaugaugtt

SEQ ID NO: 354 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaauaaugaaggccugacgtt

SEQ ID NO: 355 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaugauugcgauggauccutt

SEQ ID NO: 356 - DNA, 21 bp, Artificial Sequence
  siRNA with 19 paired ribonucleotides and two unpaired nucleotides(“t”) on the 3′ end of each strand used in screens of DNA damaging agents doxorubicin screen
aacaucaucaaccugcuggtt

SEQ ID NO: 357 - DNA, 21 bp, Artificial Sequence
  siRNA with 19 paired ribonucleotides and two unpaired nucleotides(“t”) on the 3′ end of each strand used in screens of DNA damaging agents doxorubicin screen
cacuuccagcauuuagcugtt

SEQ ID NO: 358 - DNA, 21 bp, Artificial Sequence
  siRNA with 19 paired ribonucleotides and two unpaired nucleotides(“t”) on the 3′ end of each strand used in screens of DNA damaging agents doxorubicin screen
cacuucuuacgcaaugcuutt

SEQ ID NO: 359 - DNA, 21 bp, Artificial Sequence
  siRNA with 19 paired ribonucleotides and two unpaired nucleotides(“t”) on the 3′ end of each strand used in screens of DNA damaging agents doxorubicin screen
cuaugcguacugcauagcatt

SEQ ID NO: 360 - DNA, 21 bp, Artificial Sequence
  siRNA with 19 paired ribonucleotides and two unpaired nucleotides(“t”) on the 3′ end of each strand used in screens of DNA damaging agents doxorubicin screen
gacaacgacacauagcuggtt

SEQ ID NO: 361 - DNA, 21 bp, Artificial Sequence
  siRNA with 19 paired ribonucleotides and two unpaired nucleotides(“t”) on the 3′ end of each strand used in screens of DNA damaging agents doxorubicin screen
uacaaggaaccucagagcctt

SEQ ID NO: 362 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccaucugcuugagcuacuutt

SEQ ID NO: 363 - DNA, 21 bp, Artificial Sequence
  siRNA with 19 paired ribonucleotides and two unpaired nucleotides(“t”) on the 3′ end of each strand used in screens of DNA damaging agents doxorubicin screen
guugacuuaccugacggactt

SEQ ID NO: 364 - DNA, 21 bp, Artificial Sequence
  siRNA with 19 paired ribonucleotides and two unpaired nucleotides(“t”) on the 3′ end of each strand used in screens of DNA damaging agents doxorubicin screen
uuggcaaaggcuccuuguatt

SEQ ID NO: 365 - DNA, 21 bp, Artificial Sequence
  siRNA with 19 paired ribonucleotides and two unpaired nucleotides(“t”) on the 3′ end of each strand used in screens of DNA damaging agents doxorubicin screen
agaaccucggcuacaacgutt

SEQ ID NO: 366 - DNA, 21 bp, Artificial Sequence
  siRNA with 19 paired ribonucleotides and two unpaired nucleotides(“t”) on the 3′ end of each strand used in screens of DNA damaging agents doxorubicin screen
uccgcaucuccaugugccatt

SEQ ID NO: 367 - DNA, 21 bp, Artificial Sequence
  siRNA with 19 paired ribonucleotides and two unpaired nucleotides(“t”) on the 3′ end of each strand used in screens of DNA damaging agents doxorubicin screen
ucggcuacaacgugaccaatt

SEQ ID NO: 368 - DNA, 21 bp, Artificial Sequence
  siRNA with 19 paired ribonucleotides and two unpaired nucleotides(“t”) on the 3′ end of each strand used in screens of DNA damaging agents doxorubicin screen
gacuuuccagaccuggcagtt

SEQ ID NO: 369 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaucggggucuucuccauctt

SEQ ID NO: 370 - DNA, 21 bp, Artificial Sequence
  siRNA with 19 paired ribonucleotides and two unpaired nucleotides(“t”) on the 3′ end of each strand used in screens of DNA damaging agents doxorubicin screen
ggacuucgcgcuggucuggtt

SEQ ID NO: 371 - DNA, 21 bp, Artificial Sequence
  siRNA with 19 paired ribonucleotides and two unpaired nucleotides(“t”) on the 3′ end of each strand used in screens of DNA damaging agents doxorubicin screen
auacguccaggcccucuuutt

SEQ ID NO: 372 - DNA, 21 bp, Artificial Sequence
  siRNA with 19 paired ribonucleotides and two unpaired nucleotides(“t”) on the 3′ end of each strand used in screens of DNA damaging agents doxorubicin screen
cgggcagaccggcauguuutt

SEQ ID NO: 373 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugcagcacuucaaggugcutt

SEQ ID NO: 374 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaaauucccaggggaucagtt

SEQ ID NO: 375 - DNA, 21 bp, Artificial Sequence
  siRNA with 19 paired ribonucleotides and two unpaired nucleotides(“t”) on the 3′ end of each strand used in screens of DNA damaging agents doxorubicin screen
gacagaccggcugcuuacatt

SEQ ID NO: 376 - DNA, 21 bp, Artificial Sequence
  siRNA with 19 paired ribonucleotides and two unpaired nucleotides(“t”) on the 3′ end of each strand used in screens of DNA damaging agents doxorubicin screen
gucacggaacugcauagugtt

SEQ ID NO: 377 - DNA, 21 bp, Artificial Sequence
  siRNA with 19 paired ribonucleotides and two unpaired nucleotides(“t”) on the 3′ end of each strand used in screens of DNA damaging agents doxorubicin screen
cggcaaagauuacuacuuutt

SEQ ID NO: 378 - DNA, 21 bp, Artificial Sequence
  siRNA with 19 paired ribonucleotides and two unpaired nucleotides(“t”) on the 3′ end of each strand used in screens of DNA damaging agents doxorubicin screen
ggagcccggccuguuugautt

SEQ ID NO: 379 - DNA, 21 bp, Artificial Sequence
  siRNA with 19 paired ribonucleotides and two unpaired nucleotides(“t”) on the 3′ end of each strand used in screens of DNA damaging agents doxorubicin screen
ucaagaaagcucaaaggactt

SEQ ID NO: 380 - DNA, 21 bp, Artificial Sequence
  siRNA with 19 paired ribonucleotides and two unpaired nucleotides(“t”) on the 3′ end of each strand used in screens of DNA damaging agents doxorubicin screen
cccagagauuuuugagggatt

SEQ ID NO: 381 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugccuucaacguaggcgautt

SEQ ID NO: 382 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugguaccuauuagggauggtt

SEQ ID NO: 383 - DNA, 21 bp, Artificial Sequence
  siRNA with 19 paired ribonucleotides and two unpaired nucleotides(“t”) on the 3′ end of each strand used in screens of DNA damaging agents doxorubicin screen
agaggacguuuucuacggctt

SEQ ID NO: 384 - DNA, 21 bp, Artificial Sequence
  siRNA with 19 paired ribonucleotides and two unpaired nucleotides(“t”) on the 3′ end of each strand used in screens of DNA damaging agents doxorubicin screen
aucuguuugcaaggggaagtt

SEQ ID NO: 385 - DNA, 21 bp, Artificial Sequence
  siRNA with 19 paired ribonucleotides and two unpaired nucleotides(“t”) on the 3′ end of each strand used in screens of DNA damaging agents doxorubicin screen
caagaucauccccaagccatt

SEQ ID NO: 386 - DNA, 21 bp, Artificial Sequence
  siRNA with 19 paired ribonucleotides and two unpaired nucleotides(“t”) on the 3′ end of each strand used in screens of DNA damaging agents doxorubicin screen
acaccuggagauaaacccutt

SEQ ID NO: 387 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccuaugggucguggaacaatt

SEQ ID NO: 388 - DNA, 21 bp, Artificial Sequence
  siRNA with 19 paired ribonucleotides and two unpaired nucleotides(“t”) on the 3′ end of each strand used in screens of DNA damaging agents doxorubicin screen
uaaccuugguacuaucgcctt

SEQ ID NO: 389 - DNA, 21 bp, Artificial Sequence
  siRNA with 19 paired ribonucleotides and two unpaired nucleotides(“t”) on the 3′ end of each strand used in screens of DNA damaging agents doxorubicin screen
cagcuaaaaccuuccucactt

SEQ ID NO: 390 - DNA, 21 bp, Artificial Sequence
  siRNA with 19 paired ribonucleotides and two unpaired nucleotides(“t”) on the 3′ end of each strand used in screens of DNA damaging agents doxorubicin screen
aacacaaaggcccacacuutt

SEQ ID NO: 391 - DNA, 21 bp, Artificial Sequence
  siRNA with 19 paired ribonucleotides and two unpaired nucleotides(“t”) on the 3′ end of each strand used in screens of DNA damaging agents doxorubicin screen
cagaguggaagacauaugctt

SEQ ID NO: 392 - DNA, 21 bp, Artificial Sequence
  siRNA with 19 paired ribonucleotides and two unpaired nucleotides(“t”) on the 3′ end of each strand used in screens of DNA damaging agents doxorubicin screen
agagggaagacuugguggutt

SEQ ID NO: 393 - DNA, 21 bp, Artificial Sequence
  siRNA with 19 paired ribonucleotides and two unpaired nucleotides(“t”) on the 3′ end of each strand used in screens of DNA damaging agents doxorubicin screen
cacucaugccaggacauugtt

SEQ ID NO: 394 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaagaaucgggacuacccatt

SEQ ID NO: 395 - DNA, 21 bp, Artificial Sequence
  siRNA with 19 paired ribonucleotides and two unpaired nucleotides(“t”) on the 3′ end of each strand used in screens of DNA damaging agents doxorubicin screen
agcauuaagaagcccagugtt

SEQ ID NO: 396 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaacaagcacucagagcagtt

SEQ ID NO: 397 - DNA, 21 bp, Artificial Sequence
  siRNA with 19 paired ribonucleotides and two unpaired nucleotides(“t”) on the 3′ end of each strand used in screens of DNA damaging agents doxorubicin screen
ucccaucaacauggugucctt

SEQ ID NO: 398 - DNA, 21 bp, Artificial Sequence
  siRNA with 19 paired ribonucleotides and two unpaired nucleotides(“t”) on the 3′ end of each strand used in screens of DNA damaging agents doxorubicin screen
cacagccguucgauguccatt

SEQ ID NO: 399 - DNA, 21 bp, Artificial Sequence
  siRNA with 19 paired ribonucleotides and two unpaired nucleotides(“t”) on the 3′ end of each strand used in screens of DNA damaging agents doxorubicin screen
guacaucgccaucgacgugtt

SEQ ID NO: 400 - DNA, 21 bp, Artificial Sequence
  siRNA with 19 paired ribonucleotides and two unpaired nucleotides(“t”) on the 3′ end of each strand used in screens of DNA damaging agents doxorubicin screen
guaccugaucgcccucuactt

SEQ ID NO: 401 - DNA, 21 bp, Artificial Sequence
  siRNA with 19 paired ribonucleotides and two unpaired nucleotides(“t”) on the 3′ end of each strand used in screens of DNA damaging agents doxorubicin screen
augcggaauuugccgugggtt

SEQ ID NO: 402 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcacaagcaccugugugagtt

SEQ ID NO: 403 - DNA, 21 bp, Artificial Sequence
  siRNA with 19 paired ribonucleotides and two unpaired nucleotides(“t”) on the 3′ end of each strand used in screens of DNA damaging agents doxorubicin screen
gucguacaccaugaucgggtt

SEQ ID NO: 404 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccugcagcaacugauuacctt

SEQ ID NO: 405 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaacuugagaagaugcgagtt

SEQ ID NO: 406 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaagaggcccacugaaguutt

SEQ ID NO: 407 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaugcaucucgugaucgcatt

SEQ ID NO: 408 - DNA, 21 bp, Artificial Sequence
  siRNA with 19 paired ribonucleotides and two unpaired nucleotides(“t”) on the 3′ end of each strand used in screens of DNA damaging agents doxorubicin screen
agacaguuggaggaaucugtt

SEQ ID NO: 409 - DNA, 21 bp, Artificial Sequence
  siRNA with 19 paired ribonucleotides and two unpaired nucleotides(“t”) on the 3′ end of each strand used in screens of DNA damaging agents doxorubicin screen
aucggcaacuuuagcgagutt

SEQ ID NO: 410 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaaaagacuccuggcugugtt

SEQ ID NO: 411 - DNA, 21 bp, Artificial Sequence
  siRNA with 19 paired ribonucleotides and two unpaired nucleotides(“t”) on the 3′ end of each strand used in screens of DNA damaging agents doxorubicin screen
ggacucugaagauguaccutt

SEQ ID NO: 412 - DNA, 21 bp, Artificial Sequence
  siRNA with 19 paired ribonucleotides and two unpaired nucleotides(“t”) on the 3′ end of each strand used in screens of DNA damaging agents doxorubicin screen
ggcauacuaguacaaguggtt

SEQ ID NO: 413 - DNA, 21 bp, Artificial Sequence
  siRNA with 19 paired ribonucleotides and two unpaired nucleotides(“t”) on the 3′ end of each strand used in screens of DNA damaging agents doxorubicin screen
accacaccuucaucaagcgtt

SEQ ID NO: 414 - DNA, 21 bp, Artificial Sequence
  siRNA with 19 paired ribonucleotides and two unpaired nucleotides(“t”) on the 3′ end of each strand used in screens of DNA damaging agents doxorubicin screen
agucagcaucgcgguucuctt

SEQ ID NO: 415 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaaggagagccucacagcatt

SEQ ID NO: 416 - DNA, 21 bp, Artificial Sequence
  siRNA with 19 paired ribonucleotides and two unpaired nucleotides(“t”) on the 3′ end of each strand used in screens of DNA damaging agents doxorubicin screen
ucacuuagcagcugagucutt

SEQ ID NO: 417 - DNA, 21 bp, Artificial Sequence
  siRNA with 19 paired ribonucleotides and two unpaired nucleotides(“t”) on the 3′ end of each strand used in screens of DNA damaging agents doxorubicin screen
uugacagcacuggucagagtt

SEQ ID NO: 418 - DNA, 21 bp, Artificial Sequence
  siRNA with 19 paired ribonucleotides and two unpaired nucleotides(“t”) on the 3′ end of each strand used in screens of DNA damaging agents doxorubicin screen
uuggcaagaacuucuuggctt

SEQ ID NO: 419 - DNA, 21 bp, Artificial Sequence
  siRNA with 19 paired ribonucleotides and two unpaired nucleotides(“t”) on the 3′ end of each strand used in screens of DNA damaging agents doxorubicin screen
agaacgaucguccaguggatt

SEQ ID NO: 420 - DNA, 21 bp, Artificial Sequence
  siRNA with 19 paired ribonucleotides and two unpaired nucleotides(“t”) on the 3′ end of each strand used in screens of DNA damaging agents doxorubicin screen
gguaccucgaugccauagutt

SEQ ID NO: 421 - DNA, 21 bp, Artificial Sequence
  siRNA with 19 paired ribonucleotides and two unpaired nucleotides(“t”) on the 3′ end of each strand used in screens of DNA damaging agents doxorubicin screen
uuuuggacuagugcggaugtt

SEQ ID NO: 422 - DNA, 21 bp, Artificial Sequence
  siRNA with 19 paired ribonucleotides and two unpaired nucleotides(“t”) on the 3′ end of each strand used in screens of DNA damaging agents doxorubicin screen
agaauuggcagcugaguugtt

SEQ ID NO: 423 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugcagcagcuauugcacuutt

SEQ ID NO: 424 - DNA, 21 bp, Artificial Sequence
  siRNA with 19 paired ribonucleotides and two unpaired nucleotides(“t”) on the 3′ end of each strand used in screens of DNA damaging agents doxorubicin screen
uguacagcuuggaaggaugtt

SEQ ID NO: 425 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaauccuaagaagaggccgtt

SEQ ID NO: 426 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaggaggucuuucauugggtt

SEQ ID NO: 427 - DNA, 21 bp, Artificial Sequence
  siRNA with 19 paired ribonucleotides and two unpaired nucleotides(“t”) on the 3′ end of each strand used in screens of DNA damaging agents doxorubicin screen
gauagucaagcuagacccatt

SEQ ID NO: 428 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaugggaugcuggcaaugatt

SEQ ID NO: 429 - DNA, 21 bp, Artificial Sequence
  siRNA with 19 paired ribonucleotides and two unpaired nucleotides(“t”) on the 3′ end of each strand used in screens of DNA damaging agents doxorubicin screen
auccuuacacgggccauaatt

SEQ ID NO: 430 - DNA, 21 bp, Artificial Sequence
  siRNA with 19 paired ribonucleotides and two unpaired nucleotides(“t”) on the 3′ end of each strand used in screens of DNA damaging agents doxorubicin screen
cuggaccucugucagaacutt

SEQ ID NO: 431 - DNA, 21 bp, Artificial Sequence
  siRNA with 19 paired ribonucleotides and two unpaired nucleotides(“t”) on the 3′ end of each strand used in screens of DNA damaging agents doxorubicin screen
cacccguacaucaaugucutt

SEQ ID NO: 432 - DNA, 21 bp, Artificial Sequence
  siRNA with 19 paired ribonucleotides and two unpaired nucleotides(“t”) on the 3′ end of each strand used in screens of DNA damaging agents doxorubicin screen
ggaauaguaugcgcagcuutt

SEQ ID NO: 433 - DNA, 21 bp, Artificial Sequence
  siRNA with 19 paired ribonucleotides and two unpaired nucleotides(“t”) on the 3′ end of each strand used in screens of DNA damaging agents doxorubicin screen
gugauucagauggagcuagtt

SEQ ID NO: 434 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaguauuaacagccuggactt

SEQ ID NO: 435 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcuaagcuagaacacgagutt

SEQ ID NO: 436 - DNA, 21 bp, Artificial Sequence
  siRNA with 19 paired ribonucleotides and two unpaired nucleotides(“t”) on the 3′ end of each strand used in screens of DNA damaging agents doxorubicin screen
uagaggauguguuucagcctt

SEQ ID NO: 437 - DNA, 21 bp, Artificial Sequence
  siRNA with 19 paired ribonucleotides and two unpaired nucleotides(“t”) on the 3′ end of each strand used in screens of DNA damaging agents doxorubicin screen
acaacagcacucuucaguctt

SEQ ID NO: 438 - DNA, 21 bp, Artificial Sequence
  siRNA with 19 paired ribonucleotides and two unpaired nucleotides(“t”) on the 3′ end of each strand used in screens of DNA damaging agents doxorubicin screen
cugcgagagcgaguuuuactt

SEQ ID NO: 439 - DNA, 21 bp, Artificial Sequence
  siRNA with 19 paired ribonucleotides and two unpaired nucleotides(“t”) on the 3′ end of each strand used in screens of DNA damaging agents doxorubicin screen
uguguauucuggagguagctt

SEQ ID NO: 440 - DNA, 21 bp, Artificial Sequence
  siRNA with 19 paired ribonucleotides and two unpaired nucleotides(“t”) on the 3′ end of each strand used in screens of DNA damaging agents doxorubicin screen
agcggaaacacgggaccaatt

SEQ ID NO: 441 - DNA, 21 bp, Artificial Sequence
  siRNA with 19 paired ribonucleotides and two unpaired nucleotides(“t”) on the 3′ end of each strand used in screens of DNA damaging agents doxorubicin screen
gugugaugugacagcccagtt

SEQ ID NO: 442 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugucacugaaagagguguctt

SEQ ID NO: 443 - DNA, 21 bp, Artificial Sequence
  siRNA with 19 paired ribonucleotides and two unpaired nucleotides(“t”) on the 3′ end of each strand used in screens of DNA damaging agents doxorubicin screen
aguugaggagguuucugcatt

SEQ ID NO: 444 - DNA, 21 bp, Artificial Sequence
  siRNA with 19 paired ribonucleotides and two unpaired nucleotides(“t”) on the 3′ end of each strand used in screens of DNA damaging agents doxorubicin screen
gacuuagagcugggaaucutt

SEQ ID NO: 445 - DNA, 21 bp, Artificial Sequence
  siRNA with 19 paired ribonucleotides and two unpaired nucleotides(“t”) on the 3′ end of each strand used in screens of DNA damaging agents doxorubicin screen
ggauuauauccagcagcuctt

SEQ ID NO: 446 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcagaugagcaaggaugcutt

SEQ ID NO: 447 - DNA, 21 bp, Artificial Sequence
  siRNA with 19 paired ribonucleotides and two unpaired nucleotides(“t”) on the 3′ end of each strand used in screens of DNA damaging agents doxorubicin screen
guacauccauguggccaaatt

SEQ ID NO: 448 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugggucaugaaagcugccatt

SEQ ID NO: 449 - DNA, 21 bp, Artificial Sequence
  siRNA with 19 paired ribonucleotides and two unpaired nucleotides(“t”) on the 3′ end of each strand used in screens of DNA damaging agents doxorubicin screen
augccucuggaguguauuctt

SEQ ID NO: 450 - DNA, 21 bp, Artificial Sequence
  siRNA with 19 paired ribonucleotides and two unpaired nucleotides(“t”) on the 3′ end of each strand used in screens of DNA damaging agents doxorubicin screen
augcgcccauccuuuucugtt

SEQ ID NO: 451 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaucugggcagugaauuagtt

SEQ ID NO: 452 - DNA, 21 bp, Artificial Sequence
  siRNA with 19 paired ribonucleotides and two unpaired nucleotides(“t”) on the 3′ end of each strand used in screens of DNA damaging agents doxorubicin screen
agaggaacucucugcaagctt

SEQ ID NO: 453 - DNA, 21 bp, Artificial Sequence
  siRNA with 19 paired ribonucleotides and two unpaired nucleotides(“t”) on the 3′ end of each strand used in screens of DNA damaging agents doxorubicin screen
cugaagaagcuacugcuugtt

SEQ ID NO: 454 - DNA, 21 bp, Artificial Sequence
  siRNA with 19 paired ribonucleotides and two unpaired nucleotides(“t”) on the 3′ end of each strand used in screens of DNA damaging agents doxorubicin screen
gacaugcgaaugacacuagtt

SEQ ID NO: 455 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaguuugugucccagacactt

SEQ ID NO: 456 - DNA, 21 bp, Artificial Sequence
  siRNA with 19 paired ribonucleotides and two unpaired nucleotides(“t”) on the 3′ end of each strand used in screens of DNA damaging agents doxorubicin screen
aauggcagugaaacacccutt

SEQ ID NO: 457 - DNA, 21 bp, Artificial Sequence
  siRNA with 19 paired ribonucleotides and two unpaired nucleotides(“t”) on the 3′ end of each strand used in screens of DNA damaging agents doxorubicin screen
augaaggagagugaucacctt

SEQ ID NO: 458 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaagcuaccgaugucagggtt

SEQ ID NO: 459 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaaucccgaguuauccacctt

SEQ ID NO: 460 - DNA, 21 bp, Artificial Sequence
  siRNA with 19 paired ribonucleotides and two unpaired nucleotides(“t”) on the 3′ end of each strand used in screens of DNA damaging agents doxorubicin screen
ggcuaaugaaaggugcuggtt

SEQ ID NO: 461 - DNA, 21 bp, Artificial Sequence
  siRNA with 19 paired ribonucleotides and two unpaired nucleotides(“t”) on the 3′ end of each strand used in screens of DNA damaging agents doxorubicin screen
aagugacauuugggcucugtt

SEQ ID NO: 462 - DNA, 21 bp, Artificial Sequence
  siRNA with 19 paired ribonucleotides and two unpaired nucleotides(“t”) on the 3′ end of each strand used in screens of DNA damaging agents doxorubicin screen
augcacgugcugcuguacutt

SEQ ID NO: 463 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaaggaccuucugauucugtt

SEQ ID NO: 464 - DNA, 21 bp, Artificial Sequence
  siRNA with 19 paired ribonucleotides and two unpaired nucleotides(“t”) on the 3′ end of each strand used in screens of DNA damaging agents doxorubicin screen
aggauucucaugggaagggtt

SEQ ID NO: 465 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaagaacaugauggggacutt

SEQ ID NO: 466 - DNA, 21 bp, Artificial Sequence
  siRNA with 19 paired ribonucleotides and two unpaired nucleotides(“t”) on the 3′ end of each strand used in screens of DNA damaging agents doxorubicin screen
gauugagcggccuguaacatt

SEQ ID NO: 467 - DNA, 21 bp, Artificial Sequence
  siRNA with 19 paired ribonucleotides and two unpaired nucleotides(“t”) on the 3′ end of each strand used in screens of DNA damaging agents doxorubicin screen
aggcaagcccugcaagaautt

SEQ ID NO: 468 - DNA, 21 bp, Artificial Sequence
  siRNA with 19 paired ribonucleotides and two unpaired nucleotides(“t”) on the 3′ end of each strand used in screens of DNA damaging agents doxorubicin screen
auaucgacgauuguccaggtt

SEQ ID NO: 469 - DNA, 21 bp, Artificial Sequence
  siRNA with 19 paired ribonucleotides and two unpaired nucleotides(“t”) on the 3′ end of each strand used in screens of DNA damaging agents doxorubicin screen
cacuuacaccugugugugctt

SEQ ID NO: 470 - DNA, 21 bp, Artificial Sequence
  siRNA with 19 paired ribonucleotides and two unpaired nucleotides(“t”) on the 3′ end of each strand used in screens of DNA damaging agents doxorubicin screen
aauggcuuccgcugccucutt

SEQ ID NO: 471 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaacauggccaagggugagtt

SEQ ID NO: 472 - DNA, 21 bp, Artificial Sequence
  siRNA with 19 paired ribonucleotides and two unpaired nucleotides(“t”) on the 3′ end of each strand used in screens of DNA damaging agents doxorubicin screen
gagucugggaccuccuucutt

SEQ ID NO: 473 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccagcacugacuacugugutt

SEQ ID NO: 474 - DNA, 21 bp, Artificial Sequence
  siRNA with 19 paired ribonucleotides and two unpaired nucleotides(“t”) on the 3′ end of each strand used in screens of DNA damaging agents doxorubicin screen
ggaacucgaugcuugucagtt

SEQ ID NO: 475 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugcgaggaagauacggagutt

SEQ ID NO: 476 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaggccggagaggucguuutt

SEQ ID NO: 477 - DNA, 21 bp, Artificial Sequence
  siRNA with 19 paired ribonucleotides and two unpaired nucleotides(“t”) on the 3′ end of each strand used in screens of DNA damaging agents doxorubicin screen
caucgacauuucugccuuctt

SEQ ID NO: 478 - DNA, 21 bp, Artificial Sequence
  siRNA with 19 paired ribonucleotides and two unpaired nucleotides(“t”) on the 3′ end of each strand used in screens of DNA damaging agents doxorubicin screen
gucacaugggcagagauagtt

SEQ ID NO: 479 - DNA, 21 bp, Artificial Sequence
  siRNA with 19 paired ribonucleotides and two unpaired nucleotides(“t”) on the 3′ end of each strand used in screens of DNA damaging agents doxorubicin screen
aagagccacuuucaagcugtt

SEQ ID NO: 480 - DNA, 21 bp, Artificial Sequence
  siRNA with 19 paired ribonucleotides and two unpaired nucleotides(“t”) on the 3′ end of each strand used in screens of DNA damaging agents doxorubicin screen
aguagcaacugcuggugautt

SEQ ID NO: 481 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccucuacagggagcagauutt

SEQ ID NO: 482 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccgcugucucgauauggautt

SEQ ID NO: 483 - DNA, 21 bp, Artificial Sequence
  siRNA with 19 paired ribonucleotides and two unpaired nucleotides(“t”) on the 3′ end of each strand used in screens of DNA damaging agents doxorubicin screen
ggaccgauuuuaccgaucctt

SEQ ID NO: 484 - DNA, 21 bp, Artificial Sequence
  siRNA with 19 paired ribonucleotides and two unpaired nucleotides(“t”) on the 3′ end of each strand used in screens of DNA damaging agents doxorubicin screen
uggauggcucugucaagcutt

SEQ ID NO: 485 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaagaaguaccagcaggugtt

SEQ ID NO: 486 - DNA, 21 bp, Artificial Sequence
  siRNA with 19 paired ribonucleotides and two unpaired nucleotides(“t”) on the 3′ end of each strand used in screens of DNA damaging agents doxorubicin screen
uccauccaccagagucuagtt

SEQ ID NO: 487 - DNA, 21 bp, Artificial Sequence
  siRNA with 19 paired ribonucleotides and two unpaired nucleotides(“t”) on the 3′ end of each strand used in screens of DNA damaging agents doxorubicin screen
uuugcugagcugcaucggatt

SEQ ID NO: 488 - DNA, 21 bp, Artificial Sequence
  siRNA with 19 paired ribonucleotides and two unpaired nucleotides(“t”) on the 3′ end of each strand used in screens of DNA damaging agents doxorubicin screen
aacccucauccacaggugatt

SEQ ID NO: 489 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccgacugaguacaucgaugtt

SEQ ID NO: 490 - DNA, 21 bp, Artificial Sequence
  siRNA with 19 paired ribonucleotides and two unpaired nucleotides(“t”) on the 3′ end of each strand used in screens of DNA damaging agents doxorubicin screen
gccagauucgacugauugutt

SEQ ID NO: 491 - DNA, 21 bp, Artificial Sequence
  siRNA with 19 paired ribonucleotides and two unpaired nucleotides(“t”) on the 3′ end of each strand used in screens of DNA damaging agents doxorubicin screen
aacgaggaauccgacauuctt

SEQ ID NO: 492 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugaugagcccauccuuucatt

SEQ ID NO: 493 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugcuucaacggauguagcatt

SEQ ID NO: 494 - DNA, 21 bp, Artificial Sequence
  siRNA with 19 paired ribonucleotides and two unpaired nucleotides(“t”) on the 3′ end of each strand used in screens of DNA damaging agents doxorubicin screen
aggugcacugcaguucaactt

SEQ ID NO: 495 - DNA, 21 bp, Artificial Sequence
  siRNA with 19 paired ribonucleotides and two unpaired nucleotides(“t”) on the 3′ end of each strand used in screens of DNA damaging agents doxorubicin screen
uggcuuugaaucuuuggcctt

SEQ ID NO: 496 - DNA, 21 bp, Artificial Sequence
  siRNA with 19 paired ribonucleotides and two unpaired nucleotides(“t”) on the 3′ end of each strand used in screens of DNA damaging agents doxorubicin screen
uucagcuaguacagguccutt

SEQ ID NO: 497 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaguucaugucagggcuggtt

SEQ ID NO: 498 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaugcgcaaauucagcgagtt

SEQ ID NO: 499 - DNA, 21 bp, Artificial Sequence
  siRNA with 19 paired ribonucleotides and two unpaired nucleotides(“t”) on the 3′ end of each strand used in screens of DNA damaging agents doxorubicin screen
caaagaugcccuucugaactt

SEQ ID NO: 500 - DNA, 21 bp, Artificial Sequence
  siRNA with 19 paired ribonucleotides and two unpaired nucleotides(“t”) on the 3′ end of each strand used in screens of DNA damaging agents doxorubicin screen
cggcuaguacgugaagagutt

SEQ ID NO: 501 - DNA, 21 bp, Artificial Sequence
  siRNA with 19 paired ribonucleotides and two unpaired nucleotides(“t”) on the 3′ end of each strand used in screens of DNA damaging agents doxorubicin screen
cuuauaguggagcuggcuatt

SEQ ID NO: 502 - DNA, 21 bp, Artificial Sequence
  siRNA with 19 paired ribonucleotides and two unpaired nucleotides(“t”) on the 3′ end of each strand used in screens of DNA damaging agents doxorubicin screen
gauagcggacauagcuccatt

SEQ ID NO: 503 - DNA, 21 bp, Artificial Sequence
  siRNA with 19 paired ribonucleotides and two unpaired nucleotides(“t”) on the 3′ end of each strand used in screens of DNA damaging agents doxorubicin screen
acuacguggaagagauggutt

SEQ ID NO: 504 - DNA, 21 bp, Artificial Sequence
  siRNA with 19 paired ribonucleotides and two unpaired nucleotides(“t”) on the 3′ end of each strand used in screens of DNA damaging agents doxorubicin screen
aggcaagaaguuccuucagtt

SEQ ID NO: 505 - DNA, 21 bp, Artificial Sequence
  siRNA with 19 paired ribonucleotides and two unpaired nucleotides(“t”) on the 3′ end of each strand used in screens of DNA damaging agents doxorubicin screen
ggagaauggugaccucagutt

SEQ ID NO: 506 - DNA, 21 bp, Artificial Sequence
  siRNA with 19 paired ribonucleotides and two unpaired nucleotides(“t”) on the 3′ end of each strand used in screens of DNA damaging agents doxorubicin screen
acagagacgccgaaaguactt

SEQ ID NO: 507 - DNA, 21 bp, Artificial Sequence
  siRNA with 19 paired ribonucleotides and two unpaired nucleotides(“t”) on the 3′ end of each strand used in screens of DNA damaging agents doxorubicin screen
cuagcgacauagaccuggutt

SEQ ID NO: 508 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcaaaugaauuggccuggctt

SEQ ID NO: 509 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaacccuuggaacagguuctt

SEQ ID NO: 510 - DNA, 21 bp, Artificial Sequence
  siRNA with 19 paired ribonucleotides and two unpaired nucleotides(“t”) on the 3′ end of each strand used in screens of DNA damaging agents doxorubicin screen
caagaugacaucugagcuctt

SEQ ID NO: 511 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugucugaucgaucaugcagtt

SEQ ID NO: 512 - DNA, 21 bp, Artificial Sequence
  siRNA with 19 paired ribonucleotides and two unpaired nucleotides(“t”) on the 3′ end of each strand used in screens of DNA damaging agents doxorubicin screen
cacaagaucgucuacagggtt

SEQ ID NO: 513 - DNA, 21 bp, Artificial Sequence
  siRNA with 19 paired ribonucleotides and two unpaired nucleotides(“t”) on the 3′ end of each strand used in screens of DNA damaging agents doxorubicin screen
caccagugaagucagcacutt

SEQ ID NO: 514 - DNA, 21 bp, Artificial Sequence
  siRNA with 19 paired ribonucleotides and two unpaired nucleotides(“t”) on the 3′ end of each strand used in screens of DNA damaging agents doxorubicin screen
gauuucaaguuccuggcggtt

SEQ ID NO: 515 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaguggcuccucauuguactt

SEQ ID NO: 516 - DNA, 21 bp, Artificial Sequence
  siRNA with 19 paired ribonucleotides and two unpaired nucleotides(“t”) on the 3′ end of each strand used in screens of DNA damaging agents doxorubicin screen
aucauucuggcaccuucagtt

SEQ ID NO: 517 - DNA, 21 bp, Artificial Sequence
  siRNA with 19 paired ribonucleotides and two unpaired nucleotides(“t”) on the 3′ end of each strand used in screens of DNA damaging agents doxorubicin screen
guaaaagcggaaguagucgtt

SEQ ID NO: 518 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaaugaagcaaggccgccatt

SEQ ID NO: 519 - DNA, 21 bp, Artificial Sequence
  siRNA with 19 paired ribonucleotides and two unpaired nucleotides(“t”) on the 3′ end of each strand used in screens of DNA damaging agents doxorubicin screen
acggaguacagacgucucatt

SEQ ID NO: 520 - DNA, 21 bp, Artificial Sequence
  siRNA with 19 paired ribonucleotides and two unpaired nucleotides(“t”) on the 3′ end of each strand used in screens of DNA damaging agents doxorubicin screen
ggaagcaaaggaccuucugtt

SEQ ID NO: 521 - DNA, 21 bp, Artificial Sequence
  siRNA with 19 paired ribonucleotides and two unpaired nucleotides(“t”) on the 3′ end of each strand used in screens of DNA damaging agents doxorubicin screen
aagacuggauccucagcagtt

SEQ ID NO: 522 - DNA, 21 bp, Artificial Sequence
  siRNA with 19 paired ribonucleotides and two unpaired nucleotides(“t”) on the 3′ end of each strand used in screens of DNA damaging agents doxorubicin screen
caagugcauccagcugaactt

SEQ ID NO: 523 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaaauuccugcacaaugggtt

SEQ ID NO: 524 - DNA, 21 bp, Artificial Sequence
  siRNA with 19 paired ribonucleotides and two unpaired nucleotides(“t”) on the 3′ end of each strand used in screens of DNA damaging agents doxorubicin screen
accaagcagccagcuauactt

SEQ ID NO: 525 - DNA, 21 bp, Artificial Sequence
  siRNA with 19 paired ribonucleotides and two unpaired nucleotides(“t”) on the 3′ end of each strand used in screens of DNA damaging agents doxorubicin screen
cuaaugacaucugggaccutt

SEQ ID NO: 526 - DNA, 21 bp, Artificial Sequence
  siRNA with 19 paired ribonucleotides and two unpaired nucleotides(“t”) on the 3′ end of each strand used in screens of DNA damaging agents doxorubicin screen
cuacgaaggaaaacgucagtt

SEQ ID NO: 527 - DNA, 21 bp, Artificial Sequence
  siRNA with 19 paired ribonucleotides and two unpaired nucleotides(“t”) on the 3′ end of each strand used in screens of DNA damaging agents doxorubicin screen
agacagugaagcugguugutt

SEQ ID NO: 528 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaauuucugaggacgagcutt

SEQ ID NO: 529 - DNA, 21 bp, Artificial Sequence
  siRNA with 19 paired ribonucleotides and two unpaired nucleotides(“t”) on the 3′ end of each strand used in screens of DNA damaging agents doxorubicin screen
ucagauuugggccagaguutt

SEQ ID NO: 530 - DNA, 21 bp, Artificial Sequence
  siRNA with 19 paired ribonucleotides and two unpaired nucleotides(“t”) on the 3′ end of each strand used in screens of DNA damaging agents doxorubicin screen
uggaggggaaugcucagaatt

SEQ ID NO: 531 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaggcagcuaaaggaagugtt

SEQ ID NO: 532 - DNA, 21 bp, Artificial Sequence
  siRNA with 19 paired ribonucleotides and two unpaired nucleotides(“t”) on the 3′ end of each strand used in screens of DNA damaging agents doxorubicin screen
uaaagauggcacuuucccgtt

SEQ ID NO: 533 - DNA, 21 bp, Artificial Sequence
  siRNA with 19 paired ribonucleotides and two unpaired nucleotides(“t”) on the 3′ end of each strand used in screens of DNA damaging agents doxorubicin screen
cuuggacgauguauuggagtt

SEQ ID NO: 534 - DNA, 21 bp, Artificial Sequence
  siRNA with 19 paired ribonucleotides and two unpaired nucleotides(“t”) on the 3′ end of each strand used in screens of DNA damaging agents doxorubicin screen
gacugaaaaugcuugggcatt

SEQ ID NO: 535 - DNA, 21 bp, Artificial Sequence
  siRNA with 19 paired ribonucleotides and two unpaired nucleotides(“t”) on the 3′ end of each strand used in screens of DNA damaging agents doxorubicin screen
ucccacacaucuugcugactt

SEQ ID NO: 536 - DNA, 21 bp, Artificial Sequence
  siRNA with 19 paired ribonucleotides and two unpaired nucleotides(“t”) on the 3′ end of each strand used in screens of DNA damaging agents doxorubicin screen
aacacccucugcaaaguugtt

SEQ ID NO: 537 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccgugguucuuuggcugcatt

SEQ ID NO: 538 - DNA, 21 bp, Artificial Sequence
  siRNA with 19 paired ribonucleotides and two unpaired nucleotides(“t”) on the 3′ end of each strand used in screens of DNA damaging agents doxorubicin screen
ucaggcuuauccgaugugctt

SEQ ID NO: 539 - DNA, 21 bp, Artificial Sequence
  siRNA with 19 paired ribonucleotides and two unpaired nucleotides(“t”) on the 3′ end of each strand used in screens of DNA damaging agents doxorubicin screen
aauuggcccucagcaugcutt

SEQ ID NO: 540 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaaggugaagcuuuugcactt

SEQ ID NO: 541 - DNA, 21 bp, Artificial Sequence
  siRNA with 19 paired ribonucleotides and two unpaired nucleotides(“t”) on the 3′ end of each strand used in screens of DNA damaging agents doxorubicin screen
gagguuucagccagugcautt

SEQ ID NO: 542 - DNA, 21 bp, Artificial Sequence
  siRNA with 19 paired ribonucleotides and two unpaired nucleotides(“t”) on the 3′ end of each strand used in screens of DNA damaging agents doxorubicin screen
aacaggaccguacugcuugtt

SEQ ID NO: 543 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaagccuuuguucaggucutt

SEQ ID NO: 544 - DNA, 21 bp, Artificial Sequence
  siRNA with 19 paired ribonucleotides and two unpaired nucleotides(“t”) on the 3′ end of each strand used in screens of DNA damaging agents doxorubicin screen
gagaggcauccuccuugaatt

SEQ ID NO: 545 - DNA, 21 bp, Artificial Sequence
  siRNA with 19 paired ribonucleotides and two unpaired nucleotides(“t”) on the 3′ end of each strand used in screens of DNA damaging agents doxorubicin screen
cacucucuaccgaagaucatt

SEQ ID NO: 546 - DNA, 21 bp, Artificial Sequence
  siRNA with 19 paired ribonucleotides and two unpaired nucleotides(“t”) on the 3′ end of each strand used in screens of DNA damaging agents doxorubicin screen
gauccuaaagguugucgactt

SEQ ID NO: 547 - DNA, 21 bp, Artificial Sequence
  siRNA with 19 paired ribonucleotides and two unpaired nucleotides(“t”) on the 3′ end of each strand used in screens of DNA damaging agents doxorubicin screen
ggaagccauuugcagugcutt

SEQ ID NO: 548 - DNA, 21 bp, Artificial Sequence
  siRNA with 19 paired ribonucleotides and two unpaired nucleotides(“t”) on the 3′ end of each strand used in screens of DNA damaging agents doxorubicin screen
aguaccaggauucuucagctt

SEQ ID NO: 549 - DNA, 21 bp, Artificial Sequence
  siRNA with 19 paired ribonucleotides and two unpaired nucleotides(“t”) on the 3′ end of each strand used in screens of DNA damaging agents doxorubicin screen
cuuaguucugggccauguatt

SEQ ID NO: 550 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaccccacugacagugauutt

SEQ ID NO: 551 - DNA, 21 bp, Artificial Sequence
  siRNA with 19 paired ribonucleotides and two unpaired nucleotides(“t”) on the 3′ end of each strand used in screens of DNA damaging agents doxorubicin screen
caaaaccaaggagaacgugtt

SEQ ID NO: 552 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccuaaggaggaccuuuuugtt

SEQ ID NO: 553 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaaaccgucaugcccgcuutt

SEQ ID NO: 554 - DNA, 21 bp, Artificial Sequence
  siRNA with 19 paired ribonucleotides and two unpaired nucleotides(“t”) on the 3′ end of each strand used in screens of DNA damaging agents doxorubicin screen
aguacacagcagguucauctt

SEQ ID NO: 555 - DNA, 21 bp, Artificial Sequence
  siRNA with 19 paired ribonucleotides and two unpaired nucleotides(“t”) on the 3′ end of each strand used in screens of DNA damaging agents doxorubicin screen
cgugaaugaggagaacccutt

SEQ ID NO: 556 - DNA, 21 bp, Artificial Sequence
  siRNA with 19 paired ribonucleotides and two unpaired nucleotides(“t”) on the 3′ end of each strand used in screens of DNA damaging agents doxorubicin screen
guuuaaggaggggccugugtt

SEQ ID NO: 557 - DNA, 21 bp, Artificial Sequence
  siRNA with 19 paired ribonucleotides and two unpaired nucleotides(“t”) on the 3′ end of each strand used in screens of DNA damaging agents doxorubicin screen
auugaccaccagguuucugtt

SEQ ID NO: 558 - DNA, 21 bp, Artificial Sequence
  siRNA with 19 paired ribonucleotides and two unpaired nucleotides(“t”) on the 3′ end of each strand used in screens of DNA damaging agents doxorubicin screen
cuuacaaaagggagcacactt

SEQ ID NO: 559 - DNA, 21 bp, Artificial Sequence
  siRNA with 19 paired ribonucleotides and two unpaired nucleotides(“t”) on the 3′ end of each strand used in screens of DNA damaging agents doxorubicin screen
uaucagaccggaccucuautt

SEQ ID NO: 560 - DNA, 21 bp, Artificial Sequence
  siRNA with 19 paired ribonucleotides and two unpaired nucleotides(“t”) on the 3′ end of each strand used in screens of DNA damaging agents doxorubicin screen
caauucgucggaggcaucatt

SEQ ID NO: 561 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcagugccugccuaugaaatt

SEQ ID NO: 562 - DNA, 21 bp, Artificial Sequence
  siRNA with 19 paired ribonucleotides and two unpaired nucleotides(“t”) on the 3′ end of each strand used in screens of DNA damaging agents doxorubicin screen
ggggaguuugcuggacuuutt

SEQ ID NO: 563 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaaccggaugcaguugagctt

SEQ ID NO: 564 - DNA, 21 bp, Artificial Sequence
  siRNA with 19 paired ribonucleotides and two unpaired nucleotides(“t”) on the 3′ end of each strand used in screens of DNA damaging agents doxorubicin screen
gccggaauacaagaacgggtt

SEQ ID NO: 565 - DNA, 21 bp, Artificial Sequence
  siRNA with 19 paired ribonucleotides and two unpaired nucleotides(“t”) on the 3′ end of each strand used in screens of DNA damaging agents doxorubicin screen
guggcucuuauccgcaugatt

SEQ ID NO: 566 - DNA, 21 bp, Artificial Sequence
  siRNA with 19 paired ribonucleotides and two unpaired nucleotides(“t”) on the 3′ end of each strand used in screens of DNA damaging agents doxorubicin screen
cgcuuauggaacgugauactt

SEQ ID NO: 567 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcaacagaauggcagcgautt

SEQ ID NO: 568 - DNA, 21 bp, Artificial Sequence
  siRNA with 19 paired ribonucleotides and two unpaired nucleotides(“t”) on the 3′ end of each strand used in screens of DNA damaging agents doxorubicin screen
guucuaaucggaucuggcutt

SEQ ID NO: 569 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaagccugcaucagagcuctt

SEQ ID NO: 570 - DNA, 21 bp, Artificial Sequence
  siRNA with 19 paired ribonucleotides and two unpaired nucleotides(“t”) on the 3′ end of each strand used in screens of DNA damaging agents doxorubicin screen
aguuaaucuccaggaacggtt

SEQ ID NO: 571 - DNA, 21 bp, Artificial Sequence
  siRNA with 19 paired ribonucleotides and two unpaired nucleotides(“t”) on the 3′ end of each strand used in screens of DNA damaging agents doxorubicin screen
uuugcucagcccaaaccuutt

SEQ ID NO: 572 - DNA, 21 bp, Artificial Sequence
  siRNA with 19 paired ribonucleotides and two unpaired nucleotides(“t”) on the 3′ end of each strand used in screens of DNA damaging agents doxorubicin screen
acacagaucugccucuaugtt

SEQ ID NO: 573 - DNA, 21 bp, Artificial Sequence
  siRNA with 19 paired ribonucleotides and two unpaired nucleotides(“t”) on the 3′ end of each strand used in screens of DNA damaging agents doxorubicin screen
cccuacaauaaaggccggutt

SEQ ID NO: 574 - DNA, 21 bp, Artificial Sequence
  siRNA with 19 paired ribonucleotides and two unpaired nucleotides(“t”) on the 3′ end of each strand used in screens of DNA damaging agents doxorubicin screen
uuaggaagguccuucagggtt

SEQ ID NO: 575 - DNA, 21 bp, Artificial Sequence
  siRNA with 19 paired ribonucleotides and two unpaired nucleotides(“t”) on the 3′ end of each strand used in screens of DNA damaging agents doxorubicin screen
agaccaguacuuccuccagtt

SEQ ID NO: 576 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccauacucagaacuccucutt

SEQ ID NO: 577 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaaaaagcaucagggggaatt

SEQ ID NO: 578 - DNA, 21 bp, Artificial Sequence
  siRNA with 19 paired ribonucleotides and two unpaired nucleotides(“t”) on the 3′ end of each strand used in screens of DNA damaging agents doxorubicin screen
agacaccuugacagaagagtt

SEQ ID NO: 579 - DNA, 21 bp, Artificial Sequence
  siRNA with 19 paired ribonucleotides and two unpaired nucleotides(“t”) on the 3′ end of each strand used in screens of DNA damaging agents doxorubicin screen
cucuggggauuucucaaugtt

SEQ ID NO: 580 - DNA, 21 bp, Artificial Sequence
  siRNA with 19 paired ribonucleotides and two unpaired nucleotides(“t”) on the 3′ end of each strand used in screens of DNA damaging agents doxorubicin screen
ggaaguaaaugcaggccagtt

SEQ ID NO: 581 - DNA, 21 bp, Artificial Sequence
  siRNA with 19 paired ribonucleotides and two unpaired nucleotides(“t”) on the 3′ end of each strand used in screens of DNA damaging agents doxorubicin screen
acagucuuaggaaucgugctt

SEQ ID NO: 582 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcacaaaagcuugucuccatt

SEQ ID NO: 583 - DNA, 21 bp, Artificial Sequence
  siRNA with 19 paired ribonucleotides and two unpaired nucleotides(“t”) on the 3′ end of each strand used in screens of DNA damaging agents doxorubicin screen
uugcagauuuugggugguctt

SEQ ID NO: 584 - DNA, 21 bp, Artificial Sequence
  siRNA with 19 paired ribonucleotides and two unpaired nucleotides(“t”) on the 3′ end of each strand used in screens of DNA damaging agents doxorubicin screen
cacccaaaagagcaagcagtt

SEQ ID NO: 585 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccucccuauucagaaagcutt

SEQ ID NO: 586 - DNA, 21 bp, Artificial Sequence
  siRNA with 19 paired ribonucleotides and two unpaired nucleotides(“t”) on the 3′ end of each strand used in screens of DNA damaging agents doxorubicin screen
gacuuugaaauuggucgcctt

SEQ ID NO: 587 - DNA, 21 bp, Artificial Sequence
  siRNA with 19 paired ribonucleotides and two unpaired nucleotides(“t”) on the 3′ end of each strand used in screens of DNA damaging agents doxorubicin screen
aacugcacagugcuguuggtt

SEQ ID NO: 588 - DNA, 21 bp, Artificial Sequence
  siRNA with 19 paired ribonucleotides and two unpaired nucleotides(“t”) on the 3′ end of each strand used in screens of DNA damaging agents doxorubicin screen
uacccaacgcacaaaugactt

SEQ ID NO: 589 - DNA, 21 bp, Artificial Sequence
  siRNA with 19 paired ribonucleotides and two unpaired nucleotides(“t”) on the 3′ end of each strand used in screens of DNA damaging agents doxorubicin screen
uggcuaguacuguauuggctt

SEQ ID NO: 590 - DNA, 21 bp, Artificial Sequence
  siRNA with 19 paired ribonucleotides and two unpaired nucleotides(“t”) on the 3′ end of each strand used in screens of DNA damaging agents doxorubicin screen
agaacugggcucugguaautt

SEQ ID NO: 591 - DNA, 21 bp, Artificial Sequence
  siRNA with 19 paired ribonucleotides and two unpaired nucleotides(“t”) on the 3′ end of each strand used in screens of DNA damaging agents doxorubicin screen
agaaguucgacacgcucugtt

SEQ ID NO: 592 - DNA, 21 bp, Artificial Sequence
  siRNA with 19 paired ribonucleotides and two unpaired nucleotides(“t”) on the 3′ end of each strand used in screens of DNA damaging agents doxorubicin screen
ggaaaaccucaucagggaatt

SEQ ID NO: 593 - DNA, 21 bp, Artificial Sequence
  siRNA with 19 paired ribonucleotides and two unpaired nucleotides(“t”) on the 3′ end of each strand used in screens of DNA damaging agents doxorubicin screen
agccgugguggagacccuutt

SEQ ID NO: 594 - DNA, 21 bp, Artificial Sequence
  siRNA with 19 paired ribonucleotides and two unpaired nucleotides(“t”) on the 3′ end of each strand used in screens of DNA damaging agents doxorubicin screen
agucaaggagaaaugcggutt

SEQ ID NO: 595 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccucgucacggaggugcgutt

SEQ ID NO: 596 - DNA, 21 bp, Artificial Sequence
  siRNA with 19 paired ribonucleotides and two unpaired nucleotides(“t”) on the 3′ end of each strand used in screens of DNA damaging agents doxorubicin screen
gacaugauucagccacagatt

SEQ ID NO: 597 - DNA, 21 bp, Artificial Sequence
  siRNA with 19 paired ribonucleotides and two unpaired nucleotides(“t”) on the 3′ end of each strand used in screens of DNA damaging agents doxorubicin screen
uuccucgagauaggccguutt

SEQ ID NO: 598 - DNA, 21 bp, Artificial Sequence
  siRNA with 19 paired ribonucleotides and two unpaired nucleotides(“t”) on the 3′ end of each strand used in screens of DNA damaging agents doxorubicin screen
uuugggaggucaguuguuctt

SEQ ID NO: 599 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccagaaauccugcaugagctt

SEQ ID NO: 600 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcagaacacuucagagcagtt

SEQ ID NO: 601 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaaaagggaucuggggaugtt

SEQ ID NO: 602 - DNA, 21 bp, Artificial Sequence
  siRNA with 19 paired ribonucleotides and two unpaired nucleotides(“t”) on the 3′ end of each strand used in screens of DNA damaging agents doxorubicin screen
cgugacguuaaugaaccugtt

SEQ ID NO: 603 - DNA, 21 bp, Artificial Sequence
  siRNA with 19 paired ribonucleotides and two unpaired nucleotides(“t”) on the 3′ end of each strand used in screens of DNA damaging agents doxorubicin screen
gagcaacggauccuacuuctt

SEQ ID NO: 604 - DNA, 21 bp, Artificial Sequence
  siRNA with 19 paired ribonucleotides and two unpaired nucleotides(“t”) on the 3′ end of each strand used in screens of DNA damaging agents doxorubicin screen
aagcacagaguugaugagatt

SEQ ID NO: 605 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccuugcugggacuuggauctt

SEQ ID NO: 606 - DNA, 21 bp, Artificial Sequence
  siRNA with 19 paired ribonucleotides and two unpaired nucleotides(“t”) on the 3′ end of each strand used in screens of DNA damaging agents doxorubicin screen
cgccgggaagaccguaauutt

SEQ ID NO: 607 - DNA, 21 bp, Artificial Sequence
  siRNA with 19 paired ribonucleotides and two unpaired nucleotides(“t”) on the 3′ end of each strand used in screens of DNA damaging agents doxorubicin screen
agaugacaagaagcaccactt

SEQ ID NO: 608 - DNA, 21 bp, Artificial Sequence
  siRNA with 19 paired ribonucleotides and two unpaired nucleotides(“t”) on the 3′ end of each strand used in screens of DNA damaging agents doxorubicin screen
aggaagguggaugauaaggtt

SEQ ID NO: 609 - DNA, 21 bp, Artificial Sequence
  siRNA with 19 paired ribonucleotides and two unpaired nucleotides(“t”) on the 3′ end of each strand used in screens of DNA damaging agents doxorubicin screen
caccuguuuccaaaccuugtt

SEQ ID NO: 610 - DNA, 21 bp, Artificial Sequence
  siRNA with 19 paired ribonucleotides and two unpaired nucleotides(“t”) on the 3′ end of each strand used in screens of DNA damaging agents doxorubicin screen
agagcuggaucaucccagatt

SEQ ID NO: 611 - DNA, 21 bp, Artificial Sequence
  siRNA with 19 paired ribonucleotides and two unpaired nucleotides(“t”) on the 3′ end of each strand used in screens of DNA damaging agents doxorubicin screen
aggcguuuauucgacgaugtt

SEQ ID NO: 612 - DNA, 21 bp, Artificial Sequence
  siRNA with 19 paired ribonucleotides and two unpaired nucleotides(“t”) on the 3′ end of each strand used in screens of DNA damaging agents doxorubicin screen
cacuugacgguugucccuutt

SEQ ID NO: 613 - DNA, 21 bp, Artificial Sequence
  siRNA with 19 paired ribonucleotides and two unpaired nucleotides(“t”) on the 3′ end of each strand used in screens of DNA damaging agents doxorubicin screen
gauccucccugucuaugagtt

SEQ ID NO: 614 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcaaagaaauuggacgaggtt

SEQ ID NO: 615 - DNA, 21 bp, Artificial Sequence
  siRNA with 19 paired ribonucleotides and two unpaired nucleotides(“t”) on the 3′ end of each strand used in screens of DNA damaging agents doxorubicin screen
ggcgaaaacaucggguuugtt

SEQ ID NO: 616 - DNA, 21 bp, Artificial Sequence
  siRNA with 19 paired ribonucleotides and two unpaired nucleotides(“t”) on the 3′ end of each strand used in screens of DNA damaging agents doxorubicin screen
gacuaacaaaggcagcugutt

SEQ ID NO: 617 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcagcuugcaugaaggagutt

SEQ ID NO: 618 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugccccuuuccaacugucutt

SEQ ID NO: 619 - DNA, 21 bp, Artificial Sequence
  siRNA with 19 paired ribonucleotides and two unpaired nucleotides(“t”) on the 3′ end of each strand used in screens of DNA damaging agents doxorubicin screen
aggaaauaggcagggugugtt

SEQ ID NO: 620 - DNA, 21 bp, Artificial Sequence
  siRNA with 19 paired ribonucleotides and two unpaired nucleotides(“t”) on the 3′ end of each strand used in screens of DNA damaging agents doxorubicin screen
cagaacccaaaaggguaagtt

SEQ ID NO: 621 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaucuggaagaccacccaatt

SEQ ID NO: 622 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaacaggagguguugaagctt

SEQ ID NO: 623 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaguggagcagaacagucgtt

SEQ ID NO: 624 - DNA, 21 bp, Artificial Sequence
  siRNA with 19 paired ribonucleotides and two unpaired nucleotides(“t”) on the 3′ end of each strand used in screens of DNA damaging agents doxorubicin screen
ggacaauccacagagaucutt

SEQ ID NO: 625 - DNA, 21 bp, Artificial Sequence
  siRNA with 19 paired ribonucleotides and two unpaired nucleotides(“t”) on the 3′ end of each strand used in screens of DNA damaging agents doxorubicin screen
aucggcucuggagaauuugtt

SEQ ID NO: 626 - DNA, 21 bp, Artificial Sequence
  siRNA with 19 paired ribonucleotides and two unpaired nucleotides(“t”) on the 3′ end of each strand used in screens of DNA damaging agents doxorubicin screen
caaggaucuccaguccacatt

SEQ ID NO: 627 - DNA, 21 bp, Artificial Sequence
  siRNA with 19 paired ribonucleotides and two unpaired nucleotides(“t”) on the 3′ end of each strand used in screens of DNA damaging agents doxorubicin screen
uguaccuguguguccaucutt

SEQ ID NO: 628 - DNA, 21 bp, Artificial Sequence
  siRNA with 19 paired ribonucleotides and two unpaired nucleotides(“t”) on the 3′ end of each strand used in screens of DNA damaging agents doxorubicin screen
acggcguuuaucuucgcuatt

SEQ ID NO: 629 - DNA, 21 bp, Artificial Sequence
  siRNA with 19 paired ribonucleotides and two unpaired nucleotides(“t”) on the 3′ end of each strand used in screens of DNA damaging agents doxorubicin screen
cccucuugccauccugaugtt

SEQ ID NO: 630 - DNA, 21 bp, Artificial Sequence
  siRNA with 19 paired ribonucleotides and two unpaired nucleotides(“t”) on the 3′ end of each strand used in screens of DNA damaging agents doxorubicin screen
cuauuuauugugcuggguctt

SEQ ID NO: 631 - DNA, 21 bp, Artificial Sequence
  siRNA with 19 paired ribonucleotides and two unpaired nucleotides(“t”) on the 3′ end of each strand used in screens of DNA damaging agents doxorubicin screen
auuugcccgcgcauuugugtt

SEQ ID NO: 632 - DNA, 21 bp, Artificial Sequence
  siRNA with 19 paired ribonucleotides and two unpaired nucleotides(“t”) on the 3′ end of each strand used in screens of DNA damaging agents doxorubicin screen
aacgggcgauuaucucuggtt

SEQ ID NO: 633 - DNA, 21 bp, Artificial Sequence
  siRNA with 19 paired ribonucleotides and two unpaired nucleotides(“t”) on the 3′ end of each strand used in screens of DNA damaging agents doxorubicin screen
agaagaugaauggucuggctt

SEQ ID NO: 634 - DNA, 21 bp, Artificial Sequence
  siRNA with 19 paired ribonucleotides and two unpaired nucleotides(“t”) on the 3′ end of each strand used in screens of DNA damaging agents doxorubicin screen
acaguugcaggaguuuucctt

SEQ ID NO: 635 - DNA, 21 bp, Artificial Sequence
  siRNA with 19 paired ribonucleotides and two unpaired nucleotides(“t”) on the 3′ end of each strand used in screens of DNA damaging agents doxorubicin screen
caaacugucaggcagcauutt

SEQ ID NO: 636 - DNA, 21 bp, Artificial Sequence
  siRNA with 19 paired ribonucleotides and two unpaired nucleotides(“t”) on the 3′ end of each strand used in screens of DNA damaging agents doxorubicin screen
gccagcuugcaagaaauugtt

SEQ ID NO: 637 - DNA, 21 bp, Artificial Sequence
  siRNA with 19 paired ribonucleotides and two unpaired nucleotides(“t”) on the 3′ end of each strand used in screens of DNA damaging agents doxorubicin screen
accgacacuuuggcuuccatt

SEQ ID NO: 638 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaugagcgcgggaauguugtt

SEQ ID NO: 639 - DNA, 21 bp, Artificial Sequence
  siRNA with 19 paired ribonucleotides and two unpaired nucleotides(“t”) on the 3′ end of each strand used in screens of DNA damaging agents doxorubicin screen
uggccgaggccuucaagcutt

SEQ ID NO: 640 - DNA, 21 bp, Artificial Sequence
  siRNA with 19 paired ribonucleotides and two unpaired nucleotides(“t”) on the 3′ end of each strand used in screens of DNA damaging agents doxorubicin screen
caucaaucacucucugcugtt

SEQ ID NO: 641 - DNA, 21 bp, Artificial Sequence
  siRNA with 19 paired ribonucleotides and two unpaired nucleotides(“t”) on the 3′ end of each strand used in screens of DNA damaging agents doxorubicin screen
cuaacccaggauguucaggtt

SEQ ID NO: 642 - DNA, 21 bp, Artificial Sequence
  siRNA with 19 paired ribonucleotides and two unpaired nucleotides(“t”) on the 3′ end of each strand used in screens of DNA damaging agents doxorubicin screen
gacacucaccaugcugaaatt

SEQ ID NO: 643 - DNA, 21 bp, Artificial Sequence
  siRNA with 19 paired ribonucleotides and two unpaired nucleotides(“t”) on the 3′ end of each strand used in screens of DNA damaging agents doxorubicin screen
aagggugacuuuguguccutt

SEQ ID NO: 644 - DNA, 21 bp, Artificial Sequence
  siRNA with 19 paired ribonucleotides and two unpaired nucleotides(“t”) on the 3′ end of each strand used in screens of DNA damaging agents doxorubicin screen
accaggaacaaaccuguugtt

SEQ ID NO: 645 - DNA, 21 bp, Artificial Sequence
  siRNA with 19 paired ribonucleotides and two unpaired nucleotides(“t”) on the 3′ end of each strand used in screens of DNA damaging agents doxorubicin screen
uuugaagguggcccuccuatt

SEQ ID NO: 646 - DNA, 21 bp, Artificial Sequence
  siRNA with 19 paired ribonucleotides and two unpaired nucleotides(“t”) on the 3′ end of each strand used in screens of DNA damaging agents doxorubicin screen
augaagccucaccaggacutt

SEQ ID NO: 647 - DNA, 21 bp, Artificial Sequence
  siRNA with 19 paired ribonucleotides and two unpaired nucleotides(“t”) on the 3′ end of each strand used in screens of DNA damaging agents doxorubicin screen
cacuuuucccucaacgaggtt

SEQ ID NO: 648 - DNA, 21 bp, Artificial Sequence
  siRNA with 19 paired ribonucleotides and two unpaired nucleotides(“t”) on the 3′ end of each strand used in screens of DNA damaging agents doxorubicin screen
uaguagcaaagcaggaaggtt

SEQ ID NO: 649 - DNA, 21 bp, Artificial Sequence
  siRNA with 19 paired ribonucleotides and two unpaired nucleotides(“t”) on the 3′ end of each strand used in screens of DNA damaging agents doxorubicin screen
guuguaggaggcagugaugtt

SEQ ID NO: 650 - DNA, 21 bp, Artificial Sequence
  siRNA with 19 paired ribonucleotides and two unpaired nucleotides(“t”) on the 3′ end of each strand used in screens of DNA damaging agents doxorubicin screen
ucucaauuuggaagucuugtt

SEQ ID NO: 651 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugaucaaugaucggauuggtt

SEQ ID NO: 652 - DNA, 21 bp, Artificial Sequence
  siRNA with 19 paired ribonucleotides and two unpaired nucleotides(“t”) on the 3′ end of each strand used in screens of DNA damaging agents doxorubicin screen
cgaucugcaggccaacauctt

SEQ ID NO: 653 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaaguaugagcagcucaagtt

SEQ ID NO: 654 - DNA, 21 bp, Artificial Sequence
  siRNA with 19 paired ribonucleotides and two unpaired nucleotides(“t”) on the 3′ end of each strand used in screens of DNA damaging agents doxorubicin screen
ggaccucuucaucaaugagtt

SEQ ID NO: 655 - DNA, 21 bp, Artificial Sequence
  siRNA with 19 paired ribonucleotides and two unpaired nucleotides(“t”) on the 3′ end of each strand used in screens of DNA damaging agents doxorubicin screen
accaggauuuggaguggautt

SEQ ID NO: 656 - DNA, 21 bp, Artificial Sequence
  siRNA with 19 paired ribonucleotides and two unpaired nucleotides(“t”) on the 3′ end of each strand used in screens of DNA damaging agents doxorubicin screen
caaggcauccguuauaucutt

SEQ ID NO: 657 - DNA, 21 bp, Artificial Sequence
  siRNA with 19 paired ribonucleotides and two unpaired nucleotides(“t”) on the 3′ end of each strand used in screens of DNA damaging agents doxorubicin screen
guggcuggauucauguucctt

SEQ ID NO: 658 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccagauccuugucuggaagtt

SEQ ID NO: 659 - DNA, 21 bp, Artificial Sequence
  siRNA with 19 paired ribonucleotides and two unpaired nucleotides(“t”) on the 3′ end of each strand used in screens of DNA damaging agents doxorubicin screen
cgacaacaagcugcugguctt

SEQ ID NO: 660 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaagcuguccauguuggagtt

SEQ ID NO: 661 - DNA, 21 bp, Artificial Sequence
  siRNA with 19 paired ribonucleotides and two unpaired nucleotides(“t”) on the 3′ end of each strand used in screens of DNA damaging agents doxorubicin screen
agccagcagauguaauuggtt

SEQ ID NO: 662 - DNA, 21 bp, Artificial Sequence
  siRNA with 19 paired ribonucleotides and two unpaired nucleotides(“t”) on the 3′ end of each strand used in screens of DNA damaging agents doxorubicin screen
cauuucaaugaggcuccagtt

SEQ ID NO: 663 - DNA, 21 bp, Artificial Sequence
  siRNA with 19 paired ribonucleotides and two unpaired nucleotides(“t”) on the 3′ end of each strand used in screens of DNA damaging agents doxorubicin screen
gucauuuacagaguggcuctt

SEQ ID NO: 664 - DNA, 21 bp, Artificial Sequence
  siRNA with 19 paired ribonucleotides and two unpaired nucleotides(“t”) on the 3′ end of each strand used in screens of DNA damaging agents doxorubicin screen
aggacacuuuggguaccagtt

SEQ ID NO: 665 - DNA, 21 bp, Artificial Sequence
  siRNA with 19 paired ribonucleotides and two unpaired nucleotides(“t”) on the 3′ end of each strand used in screens of DNA damaging agents doxorubicin screen
gacccuaaagaucguccuutt

SEQ ID NO: 666 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcugaggagaauaugccuctt

SEQ ID NO: 667 - DNA, 21 bp, Artificial Sequence
  siRNA with 19 paired ribonucleotides and two unpaired nucleotides(“t”) on the 3′ end of each strand used in screens of DNA damaging agents doxorubicin screen
cguguacuacaacgaggcctt

SEQ ID NO: 668 - DNA, 21 bp, Artificial Sequence
  siRNA with 19 paired ribonucleotides and two unpaired nucleotides(“t”) on the 3′ end of each strand used in screens of DNA damaging agents doxorubicin screen
uccccucugacuccaacuutt

SEQ ID NO: 669 - DNA, 21 bp, Artificial Sequence
  siRNA with 19 paired ribonucleotides and two unpaired nucleotides(“t”) on the 3′ end of each strand used in screens of DNA damaging agents doxorubicin screen
cgaggcacucuacgacauctt

SEQ ID NO: 670 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcaaugaggacagcuugugtt

SEQ ID NO: 671 - DNA, 21 bp, Artificial Sequence
  siRNA with 19 paired ribonucleotides and two unpaired nucleotides(“t”) on the 3′ end of each strand used in screens of DNA damaging agents doxorubicin screen
ucuccuugugaacagcaactt

SEQ ID NO: 672 - DNA, 21 bp, Artificial Sequence
  siRNA with 19 paired ribonucleotides and two unpaired nucleotides(“t”) on the 3′ end of each strand used in screens of DNA damaging agents doxorubicin screen
uguagcuuuccacuggagutt

SEQ ID NO: 673 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaggucuuuacgccaguactt

SEQ ID NO: 674 - DNA, 21 bp, Artificial Sequence
  siRNA with 19 paired ribonucleotides and two unpaired nucleotides(“t”) on the 3′ end of each strand used in screens of DNA damaging agents doxorubicin screen
ggaauguauccgagcacugtt

SEQ ID NO: 675 - DNA, 21 bp, Artificial Sequence
  siRNA with 19 paired ribonucleotides and two unpaired nucleotides(“t”) on the 3′ end of each strand used in screens of DNA damaging agents doxorubicin screen
uaagccugguggugaucuutt

SEQ ID NO: 676 - DNA, 21 bp, Artificial Sequence
  siRNA with 19 paired ribonucleotides and two unpaired nucleotides(“t”) on the 3′ end of each strand used in screens of DNA damaging agents doxorubicin screen
cucaagggaaauauccgugtt

SEQ ID NO: 677 - DNA, 21 bp, Artificial Sequence
  siRNA with 19 paired ribonucleotides and two unpaired nucleotides(“t”) on the 3′ end of each strand used in screens of DNA damaging agents doxorubicin screen
guguguugugccugcugaatt

SEQ ID NO: 678 - DNA, 21 bp, Artificial Sequence
  siRNA with 19 paired ribonucleotides and two unpaired nucleotides(“t”) on the 3′ end of each strand used in screens of DNA damaging agents doxorubicin screen
ucaggcauggcauuaaaactt

SEQ ID NO: 679 - DNA, 21 bp, Artificial Sequence
  siRNA with 19 paired ribonucleotides and two unpaired nucleotides(“t”) on the 3′ end of each strand used in screens of DNA damaging agents doxorubicin screen
caaaguuauuagccccaagtt

SEQ ID NO: 680 - DNA, 21 bp, Artificial Sequence
  siRNA with 19 paired ribonucleotides and two unpaired nucleotides(“t”) on the 3′ end of each strand used in screens of DNA damaging agents doxorubicin screen
cagaggccaaguauaucaatt

SEQ ID NO: 681 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccugcagauuugcacagcgtt

SEQ ID NO: 682 - DNA, 21 bp, Artificial Sequence
  siRNA with 19 paired ribonucleotides and two unpaired nucleotides(“t”) on the 3′ end of each strand used in screens of DNA damaging agents doxorubicin screen
auccuggagaugaccgugatt

SEQ ID NO: 683 - DNA, 21 bp, Artificial Sequence
  siRNA with 19 paired ribonucleotides and two unpaired nucleotides(“t”) on the 3′ end of each strand used in screens of DNA damaging agents doxorubicin screen
gccggucauggagaagcggtt

SEQ ID NO: 684 - DNA, 21 bp, Artificial Sequence
  siRNA with 19 paired ribonucleotides and two unpaired nucleotides(“t”) on the 3′ end of each strand used in screens of DNA damaging agents doxorubicin screen
uggcccugagacugcaucgtt

SEQ ID NO: 685 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccccuccaugcucagaacutt

SEQ ID NO: 686 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccuaucugggaagccugugtt

SEQ ID NO: 687 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugccccagugacaauaacatt

SEQ ID NO: 688 - DNA, 21 bp, Artificial Sequence
  siRNA with 19 paired ribonucleotides and two unpaired nucleotides(“t”) on the 3′ end of each strand used in screens of DNA damaging agents doxorubicin screen
accagggccaaaauuauggtt

SEQ ID NO: 689 - DNA, 21 bp, Artificial Sequence
  siRNA with 19 paired ribonucleotides and two unpaired nucleotides(“t”) on the 3′ end of each strand used in screens of DNA damaging agents doxorubicin screen
auagugaaccuggaacuggtt

SEQ ID NO: 690 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcaguugccccagaaguuutt

SEQ ID NO: 691 - DNA, 21 bp, Artificial Sequence
  siRNA with 19 paired ribonucleotides and two unpaired nucleotides(“t”) on the 3′ end of each strand used in screens of DNA damaging agents doxorubicin screen
agaacacacugcuugguugtt

SEQ ID NO: 692 - DNA, 21 bp, Artificial Sequence
  siRNA with 19 paired ribonucleotides and two unpaired nucleotides(“t”) on the 3′ end of each strand used in screens of DNA damaging agents doxorubicin screen
gacagugaacauggaaccatt

SEQ ID NO: 693 - DNA, 21 bp, Artificial Sequence
  siRNA with 19 paired ribonucleotides and two unpaired nucleotides(“t”) on the 3′ end of each strand used in screens of DNA damaging agents doxorubicin screen
gagaacuugcagcacacactt

SEQ ID NO: 694 - DNA, 21 bp, Artificial Sequence
  siRNA with 19 paired ribonucleotides and two unpaired nucleotides(“t”) on the 3′ end of each strand used in screens of DNA damaging agents doxorubicin screen
accugccaaccugcucauctt

SEQ ID NO: 695 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaucuccuuuaaggagcagtt

SEQ ID NO: 696 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcagcuguguauuuaaggatt

SEQ ID NO: 697 - DNA, 21 bp, Artificial Sequence
  siRNA with 19 paired ribonucleotides and two unpaired nucleotides(“t”) on the 3′ end of each strand used in screens of DNA damaging agents doxorubicin screen
agacaaagaggggaccuuctt

SEQ ID NO: 698 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaaagucuauccgaaggcutt

SEQ ID NO: 699 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugccucccugaaacuucgatt

SEQ ID NO: 700 - DNA, 21 bp, Artificial Sequence
  siRNA with 19 paired ribonucleotides and two unpaired nucleotides(“t”) on the 3′ end of each strand used in screens of DNA damaging agents doxorubicin screen
agguaugcaucgugcagaatt

SEQ ID NO: 701 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcaaucaaaccgucaugactt

SEQ ID NO: 702 - DNA, 21 bp, Artificial Sequence
  siRNA with 19 paired ribonucleotides and two unpaired nucleotides(“t”) on the 3′ end of each strand used in screens of DNA damaging agents doxorubicin screen
uauccugcuggaugaacactt

SEQ ID NO: 703 - DNA, 21 bp, Artificial Sequence
  siRNA with 19 paired ribonucleotides and two unpaired nucleotides(“t”) on the 3′ end of each strand used in screens of DNA damaging agents doxorubicin screen
gcaagguauacaaugccgutt

SEQ ID NO: 704 - DNA, 21 bp, Artificial Sequence
  siRNA with 19 paired ribonucleotides and two unpaired nucleotides(“t”) on the 3′ end of each strand used in screens of DNA damaging agents doxorubicin screen
uaacucagcaacccagcaatt

SEQ ID NO: 705 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugccuugaagacaguaccatt

SEQ ID NO: 706 - DNA, 21 bp, Artificial Sequence
  siRNA with 19 paired ribonucleotides and two unpaired nucleotides(“t”) on the 3′ end of each strand used in screens of DNA damaging agents doxorubicin screen
ccucagccguauaauacgutt

SEQ ID NO: 707 - DNA, 21 bp, Artificial Sequence
  siRNA with 19 paired ribonucleotides and two unpaired nucleotides(“t”) on the 3′ end of each strand used in screens of DNA damaging agents doxorubicin screen
cugcucuguucaaucccagtt

SEQ ID NO: 708 - DNA, 21 bp, Artificial Sequence
  siRNA with 19 paired ribonucleotides and two unpaired nucleotides(“t”) on the 3′ end of each strand used in screens of DNA damaging agents doxorubicin screen
cugggauuggccaccucuutt

SEQ ID NO: 709 - DNA, 21 bp, Artificial Sequence
  siRNA with 19 paired ribonucleotides and two unpaired nucleotides(“t”) on the 3′ end of each strand used in screens of DNA damaging agents doxorubicin screen
agaaggagagugucagguutt

SEQ ID NO: 710 - DNA, 21 bp, Artificial Sequence
  siRNA with 19 paired ribonucleotides and two unpaired nucleotides(“t”) on the 3′ end of each strand used in screens of DNA damaging agents doxorubicin screen
uauguaccccguucagcaatt

SEQ ID NO: 711 - DNA, 21 bp, Artificial Sequence
  siRNA with 19 paired ribonucleotides and two unpaired nucleotides(“t”) on the 3′ end of each strand used in screens of DNA damaging agents doxorubicin screen
uuuugccuuggagugcucctt

SEQ ID NO: 712 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaaacccccgggagucguctt

SEQ ID NO: 713 - DNA, 21 bp, Artificial Sequence
  siRNA with 19 paired ribonucleotides and two unpaired nucleotides(“t”) on the 3′ end of each strand used in screens of DNA damaging agents doxorubicin screen
aguggcaccaaguggcuggtt

SEQ ID NO: 714 - DNA, 21 bp, Artificial Sequence
  siRNA with 19 paired ribonucleotides and two unpaired nucleotides(“t”) on the 3′ end of each strand used in screens of DNA damaging agents doxorubicin screen
gaaaccugcuuugucauuutt

SEQ ID NO: 715 - DNA, 21 bp, Artificial Sequence
  siRNA with 19 paired ribonucleotides and two unpaired nucleotides(“t”) on the 3′ end of each strand used in screens of DNA damaging agents doxorubicin screen
cugaugcacuuugcugcagtt

SEQ ID NO: 716 - DNA, 21 bp, Artificial Sequence
  siRNA with 19 paired ribonucleotides and two unpaired nucleotides(“t”) on the 3′ end of each strand used in screens of DNA damaging agents doxorubicin screen
cugcagguucaaaucccagtt

SEQ ID NO: 717 - DNA, 21 bp, Artificial Sequence
  siRNA with 19 paired ribonucleotides and two unpaired nucleotides(“t”) on the 3′ end of each strand used in screens of DNA damaging agents doxorubicin screen
ggggaaaaagcuuugcguutt

SEQ ID NO: 718 - DNA, 21 bp, Artificial Sequence
  siRNA with 19 paired ribonucleotides and two unpaired nucleotides(“t”) on the 3′ end of each strand used in screens of DNA damaging agents doxorubicin screen
aaagacccaggcuaucagatt

SEQ ID NO: 719 - DNA, 21 bp, Artificial Sequence
  siRNA with 19 paired ribonucleotides and two unpaired nucleotides(“t”) on the 3′ end of each strand used in screens of DNA damaging agents doxorubicin screen
cagaccccaaauccauaagtt

SEQ ID NO: 720 - DNA, 21 bp, Artificial Sequence
  siRNA with 19 paired ribonucleotides and two unpaired nucleotides(“t”) on the 3′ end of each strand used in screens of DNA damaging agents doxorubicin screen
gucagugucacccagcaaatt

SEQ ID NO: 721 - DNA, 21 bp, Artificial Sequence
  siRNA with 19 paired ribonucleotides and two unpaired nucleotides(“t”) on the 3′ end of each strand used in screens of DNA damaging agents doxorubicin screen
uaucgagccaccauuugugtt

SEQ ID NO: 722 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugaugcauauagccgcaactt

SEQ ID NO: 723 - DNA, 21 bp, Artificial Sequence
  siRNA with 19 paired ribonucleotides and two unpaired nucleotides(“t”) on the 3′ end of each strand used in screens of DNA damaging agents doxorubicin screen
ugcacagggccaaaguugatt

SEQ ID NO: 724 - DNA, 21 bp, Artificial Sequence
  siRNA with 19 paired ribonucleotides and two unpaired nucleotides(“t”) on the 3′ end of each strand used in screens of DNA damaging agents camptothecin screen
cgcaagaagaaggccgugutt

SEQ ID NO: 725 - DNA, 21 bp, Artificial Sequence
  siRNA with 19 paired ribonucleotides and two unpaired nucleotides(“t”) on the 3′ end of each strand used in screens of DNA damaging agents camptothecin screen
cgcuggugcaauguuuucutt

SEQ ID NO: 726 - DNA, 21 bp, Artificial Sequence
  siRNA with 19 paired ribonucleotides and two unpaired nucleotides(“t”) on the 3′ end of each strand used in screens of DNA damaging agents camptothecin screen
gaaucccuaccgagacucutt

SEQ ID NO: 727 - DNA, 21 bp, Artificial Sequence
  siRNA with 19 paired ribonucleotides and two unpaired nucleotides(“t”) on the 3′ end of each strand used in screens of DNA damaging agents camptothecin screen
aaaccucuacacguucugctt

SEQ ID NO: 728 - DNA, 21 bp, Artificial Sequence
  siRNA with 19 paired ribonucleotides and two unpaired nucleotides(“t”) on the 3′ end of each strand used in screens of DNA damaging agents camptothecin screen
cuaaaggugaaaagcuccgtt

SEQ ID NO: 729 - DNA, 21 bp, Artificial Sequence
  siRNA with 19 paired ribonucleotides and two unpaired nucleotides(“t”) on the 3′ end of each strand used in screens of DNA damaging agents camptothecin screen
uccuggcaagaaagcuugatt

SEQ ID NO: 730 - DNA, 21 bp, Artificial Sequence
  siRNA with 19 paired ribonucleotides and two unpaired nucleotides(“t”) on the 3′ end of each strand used in screens of DNA damaging agents camptothecin screen
aucagugauguggugcagatt

SEQ ID NO: 731 - DNA, 21 bp, Artificial Sequence
  siRNA with 19 paired ribonucleotides and two unpaired nucleotides(“t”) on the 3′ end of each strand used in screens of DNA damaging agents camptothecin screen
gacucggacacugaagaaatt

SEQ ID NO: 732 - DNA, 21 bp, Artificial Sequence
  siRNA with 19 paired ribonucleotides and two unpaired nucleotides(“t”) on the 3′ end of each strand used in screens of DNA damaging agents camptothecin screen
uggcacagcagguacuaaatt

SEQ ID NO: 733 - DNA, 21 bp, Artificial Sequence
  siRNA with 19 paired ribonucleotides and two unpaired nucleotides(“t”) on the 3′ end of each strand used in screens of DNA damaging agents camptothecin screen
aagauggccacaaaccugctt

SEQ ID NO: 734 - DNA, 21 bp, Artificial Sequence
  siRNA with 19 paired ribonucleotides and two unpaired nucleotides(“t”) on the 3′ end of each strand used in screens of DNA damaging agents camptothecin screen
agauaaacggcggcauugutt

SEQ ID NO: 735 - DNA, 21 bp, Artificial Sequence
  siRNA with 19 paired ribonucleotides and two unpaired nucleotides(“t”) on the 3′ end of each strand used in screens of DNA damaging agents camptothecin screen
gacaugcaggaaguuguugtt

SEQ ID NO: 736 - DNA, 21 bp, Artificial Sequence
  siRNA with 19 paired ribonucleotides and two unpaired nucleotides(“t”) on the 3′ end of each strand used in screens of DNA damaging agents camptothecin screen
cgggagaucuucagcacactt

SEQ ID NO: 737 - DNA, 21 bp, Artificial Sequence
  siRNA with 19 paired ribonucleotides and two unpaired nucleotides(“t”) on the 3′ end of each strand used in screens of DNA damaging agents camptothecin screen
gagauuggccagcacccuutt

SEQ ID NO: 738 - DNA, 21 bp, Artificial Sequence
  siRNA with 19 paired ribonucleotides and two unpaired nucleotides(“t”) on the 3′ end of each strand used in screens of DNA damaging agents camptothecin screen
gcccaggacaugagugucutt

SEQ ID NO: 739 - DNA, 21 bp, Artificial Sequence
  siRNA with 19 paired ribonucleotides and two unpaired nucleotides(“t”) on the 3′ end of each strand used in screens of DNA damaging agents camptothecin screen
agauggccacaucaagauctt

SEQ ID NO: 740 - DNA, 21 bp, Artificial Sequence
  siRNA with 19 paired ribonucleotides and two unpaired nucleotides(“t”) on the 3′ end of each strand used in screens of DNA damaging agents camptothecin screen
gucaucauugccaaggaugtt

SEQ ID NO: 741 - DNA, 21 bp, Artificial Sequence
  siRNA with 19 paired ribonucleotides and two unpaired nucleotides(“t”) on the 3′ end of each strand used in screens of DNA damaging agents camptothecin screen
ugccagcugaugaagaccgtt

SEQ ID NO: 742 - DNA, 21 bp, Artificial Sequence
  siRNA with 19 paired ribonucleotides and two unpaired nucleotides(“t”) on the 3′ end of each strand used in screens of DNA damaging agents camptothecin screen
acagugcugaacuuggcuutt

SEQ ID NO: 743 - DNA, 21 bp, Artificial Sequence
  siRNA with 19 paired ribonucleotides and two unpaired nucleotides(“t”) on the 3′ end of each strand used in screens of DNA damaging agents camptothecin screen
ccaaaugucagaggcacautt

SEQ ID NO: 744 - DNA, 21 bp, Artificial Sequence
  siRNA with 19 paired ribonucleotides and two unpaired nucleotides(“t”) on the 3′ end of each strand used in screens of DNA damaging agents camptothecin screen
ucaaacugauggcugaaggtt

SEQ ID NO: 745 - DNA, 21 bp, Artificial Sequence
  siRNA with 19 paired ribonucleotides and two unpaired nucleotides(“t”) on the 3′ end of each strand used in screens of DNA damaging agents camptothecin screen
gaaagugagcgacgaguuutt

SEQ ID NO: 746 - DNA, 21 bp, Artificial Sequence
  siRNA with 19 paired ribonucleotides and two unpaired nucleotides(“t”) on the 3′ end of each strand used in screens of DNA damaging agents camptothecin screen
gugccuucaacacagcuugtt

SEQ ID NO: 747 - DNA, 21 bp, Artificial Sequence
  siRNA with 19 paired ribonucleotides and two unpaired nucleotides(“t”) on the 3′ end of each strand used in screens of DNA damaging agents camptothecin screen
ugaauauccaagagcagggtt

SEQ ID NO: 748 - DNA, 21 bp, Artificial Sequence
  siRNA with 19 paired ribonucleotides and two unpaired nucleotides(“t”) on the 3′ end of each strand used in screens of DNA damaging agents camptothecin screen
aggacauuuguuggaggggtt

SEQ ID NO: 749 - DNA, 21 bp, Artificial Sequence
  siRNA with 19 paired ribonucleotides and two unpaired nucleotides(“t”) on the 3′ end of each strand used in screens of DNA damaging agents camptothecin screen
gcuaccaauugugccgagatt

SEQ ID NO: 750 - DNA, 21 bp, Artificial Sequence
  siRNA with 19 paired ribonucleotides and two unpaired nucleotides(“t”) on the 3′ end of each strand used in screens of DNA damaging agents camptothecin screen
ugaagaacguggacgcuuutt

SEQ ID NO: 751 - DNA, 21 bp, Artificial Sequence
  siRNA with 19 paired ribonucleotides and two unpaired nucleotides(“t”) on the 3′ end of each strand used in screens of DNA damaging agents camptothecin screen
agucacgagcguucgaaaatt

SEQ ID NO: 752 - DNA, 21 bp, Artificial Sequence
  siRNA with 19 paired ribonucleotides and two unpaired nucleotides(“t”) on the 3′ end of each strand used in screens of DNA damaging agents camptothecin screen
caacaugaagguggccauutt

SEQ ID NO: 753 - DNA, 21 bp, Artificial Sequence
  siRNA with 19 paired ribonucleotides and two unpaired nucleotides(“t”) on the 3′ end of each strand used in screens of DNA damaging agents camptothecin screen
gcacuauaagauccgcugctt

SEQ ID NO: 754 - DNA, 21 bp, Artificial Sequence
  siRNA with 19 paired ribonucleotides and two unpaired nucleotides(“t”) on the 3′ end of each strand used in screens of DNA damaging agents camptothecin screen
acagauuggaaaaggucgctt

SEQ ID NO: 755 - DNA, 21 bp, Artificial Sequence
  siRNA with 19 paired ribonucleotides and two unpaired nucleotides(“t”) on the 3′ end of each strand used in screens of DNA damaging agents camptothecin screen
gaaguuacgccccucauuctt

SEQ ID NO: 756 - DNA, 21 bp, Artificial Sequence
  siRNA with 19 paired ribonucleotides and two unpaired nucleotides(“t”) on the 3′ end of each strand used in screens of DNA damaging agents camptothecin screen
uauuugcagcacagacggatt

SEQ ID NO: 757 - DNA, 21 bp, Artificial Sequence
  siRNA with 19 paired ribonucleotides and two unpaired nucleotides(“t”) on the 3′ end of each strand used in screens of DNA damaging agents camptothecin screen
caaaucugugagcccaacatt

SEQ ID NO: 758 - DNA, 21 bp, Artificial Sequence
  siRNA with 19 paired ribonucleotides and two unpaired nucleotides(“t”) on the 3′ end of each strand used in screens of DNA damaging agents camptothecin screen
caagauguucuugcacaggtt

SEQ ID NO: 759 - DNA, 21 bp, Artificial Sequence
  siRNA with 19 paired ribonucleotides and two unpaired nucleotides(“t”) on the 3′ end of each strand used in screens of DNA damaging agents camptothecin screen
gaacggcuaugugcguuuatt

SEQ ID NO: 760 - DNA, 21 bp, Artificial Sequence
  siRNA with 19 paired ribonucleotides and two unpaired nucleotides(“t”) on the 3′ end of each strand used in screens of DNA damaging agents camptothecin screen
acuuaggugaagcagcauctt

SEQ ID NO: 761 - DNA, 21 bp, Artificial Sequence
  siRNA with 19 paired ribonucleotides and two unpaired nucleotides(“t”) on the 3′ end of each strand used in screens of DNA damaging agents camptothecin screen
gggcagugaagacuugauutt

SEQ ID NO: 762 - DNA, 21 bp, Artificial Sequence
  siRNA with 19 paired ribonucleotides and two unpaired nucleotides(“t”) on the 3′ end of each strand used in screens of DNA damaging agents camptothecin screen
ugaagugggcuccaguauutt

SEQ ID NO: 763 - DNA, 21 bp, Artificial Sequence
  siRNA with 19 paired ribonucleotides and two unpaired nucleotides(“t”) on the 3′ end of each strand used in screens of DNA damaging agents camptothecin screen
caaaugggcaggacucuuatt

SEQ ID NO: 764 - DNA, 21 bp, Artificial Sequence
  siRNA with 19 paired ribonucleotides and two unpaired nucleotides(“t”) on the 3′ end of each strand used in screens of DNA damaging agents camptothecin screen
cuguucagcccaguuugaatt

SEQ ID NO: 765 - DNA, 21 bp, Artificial Sequence
  siRNA with 19 paired ribonucleotides and two unpaired nucleotides(“t”) on the 3′ end of each strand used in screens of DNA damaging agents camptothecin screen
ucuccaaggaaguuguacctt

SEQ ID NO: 766 - DNA, 21 bp, Artificial Sequence
  siRNA with 19 paired ribonucleotides and two unpaired nucleotides(“t”) on the 3′ end of each strand used in screens of DNA damaging agents camptothecin screen
agucccaggugggacucuutt

SEQ ID NO: 767 - DNA, 21 bp, Artificial Sequence
  siRNA with 19 paired ribonucleotides and two unpaired nucleotides(“t”) on the 3′ end of each strand used in screens of DNA damaging agents camptothecin screen
cuggcauccuugagcugactt

SEQ ID NO: 768 - DNA, 21 bp, Artificial Sequence
  siRNA with 19 paired ribonucleotides and two unpaired nucleotides(“t”) on the 3′ end of each strand used in screens of DNA damaging agents camptothecin screen
gacuguucuggaaugagggtt

SEQ ID NO: 769 - DNA, 21 bp, Artificial Sequence
  siRNA with 19 paired ribonucleotides and two unpaired nucleotides(“t”) on the 3′ end of each strand used in screens of DNA damaging agents camptothecin screen
accagauggaguaaaggagtt

SEQ ID NO: 770 - DNA, 21 bp, Artificial Sequence
  siRNA with 19 paired ribonucleotides and two unpaired nucleotides(“t”) on the 3′ end of each strand used in screens of DNA damaging agents camptothecin screen
gcacccuaauauugugcgatt

SEQ ID NO: 771 - DNA, 21 bp, Artificial Sequence
  siRNA with 19 paired ribonucleotides and two unpaired nucleotides(“t”) on the 3′ end of each strand used in screens of DNA damaging agents camptothecin screen
uuggcagacuuuggcuuagtt

SEQ ID NO: 772 - DNA, 21 bp, Artificial Sequence
  siRNA with 19 paired ribonucleotides and two unpaired nucleotides(“t”) on the 3′ end of each strand used in screens of DNA damaging agents camptothecin screen
cauguaaccggcauguuuctt

SEQ ID NO: 773 - DNA, 21 bp, Artificial Sequence
  siRNA with 19 paired ribonucleotides and two unpaired nucleotides(“t”) on the 3′ end of each strand used in screens of DNA damaging agents camptothecin screen
cccacagcuacuugguuugtt

SEQ ID NO: 774 - DNA, 21 bp, Artificial Sequence
  siRNA with 19 paired ribonucleotides and two unpaired nucleotides(“t”) on the 3′ end of each strand used in screens of DNA damaging agents camptothecin screen
ugaccccgcacgauuucautt

SEQ ID NO: 775 - DNA, 21 bp, Artificial Sequence
  siRNA with 19 paired ribonucleotides and two unpaired nucleotides(“t”) on the 3′ end of each strand used in screens of DNA damaging agents camptothecin screen
ccacagaugagguccauactt

SEQ ID NO: 776 - DNA, 21 bp, Artificial Sequence
  siRNA with 19 paired ribonucleotides and two unpaired nucleotides(“t”) on the 3′ end of each strand used in screens of DNA damaging agents camptothecin screen
cuggggcuuucuugacaugtt

SEQ ID NO: 777 - DNA, 21 bp, Artificial Sequence
  siRNA with 19 paired ribonucleotides and two unpaired nucleotides(“t”) on the 3′ end of each strand used in screens of DNA damaging agents camptothecin screen
gugguuaagaaagccucagtt

SEQ ID NO: 778 - DNA, 21 bp, Artificial Sequence
  siRNA with 19 paired ribonucleotides and two unpaired nucleotides(“t”) on the 3′ end of each strand used in screens of DNA damaging agents camptothecin screen
agcgccaguaaagaagaactt

SEQ ID NO: 779 - DNA, 21 bp, Artificial Sequence
  siRNA with 19 paired ribonucleotides and two unpaired nucleotides(“t”) on the 3′ end of each strand used in screens of DNA damaging agents camptothecin screen
agggcagccaguugucauutt

SEQ ID NO: 780 - DNA, 21 bp, Artificial Sequence
  siRNA with 19 paired ribonucleotides and two unpaired nucleotides(“t”) on the 3′ end of each strand used in screens of DNA damaging agents camptothecin screen
ccaccacuccaaaaugagctt

SEQ ID NO: 781 - DNA, 21 bp, Artificial Sequence
  siRNA with 19 paired ribonucleotides and two unpaired nucleotides(“t”) on the 3′ end of each strand used in screens of DNA damaging agents camptothecin screen
ggccauccccuccauuaautt

SEQ ID NO: 782 - DNA, 21 bp, Artificial Sequence
  siRNA with 19 paired ribonucleotides and two unpaired nucleotides(“t”) on the 3′ end of each strand used in screens of DNA damaging agents camptothecin screen
guaauugaaaggcagugcctt

SEQ ID NO: 783 - DNA, 21 bp, Artificial Sequence
  siRNA with 19 paired ribonucleotides and two unpaired nucleotides(“t”) on the 3′ end of each strand used in screens of DNA damaging agents camptothecin screen
uugcuaucacuguggcucutt

SEQ ID NO: 784 - DNA, 21 bp, Artificial Sequence
  siRNA with 19 paired ribonucleotides and two unpaired nucleotides(“t”) on the 3′ end of each strand used in screens of DNA damaging agents camptothecin screen
aguggcuucaaagaucccutt

SEQ ID NO: 785 - DNA, 21 bp, Artificial Sequence
  siRNA with 19 paired ribonucleotides and two unpaired nucleotides(“t”) on the 3′ end of each strand used in screens of DNA damaging agents camptothecin screen
gcaugucguuaccuugcagtt

SEQ ID NO: 786 - DNA, 21 bp, Artificial Sequence
  siRNA with 19 paired ribonucleotides and two unpaired nucleotides(“t”) on the 3′ end of each strand used in screens of DNA damaging agents camptothecin screen
uaagccuagugaaacgguctt

SEQ ID NO: 787 - DNA, 21 bp, Artificial Sequence
  siRNA with 19 paired ribonucleotides and two unpaired nucleotides(“t”) on the 3′ end of each strand used in screens of DNA damaging agents camptothecin screen
agcaacugcugcuuauugctt

SEQ ID NO: 788 - DNA, 21 bp, Artificial Sequence
  siRNA with 19 paired ribonucleotides and two unpaired nucleotides(“t”) on the 3′ end of each strand used in screens of DNA damaging agents camptothecin screen
agcaagcaaggagauaggatt

SEQ ID NO: 789 - DNA, 21 bp, Artificial Sequence
  siRNA with 19 paired ribonucleotides and two unpaired nucleotides(“t”) on the 3′ end of each strand used in screens of DNA damaging agents camptothecin screen
ccuucuuuaugucaggagctt

SEQ ID NO: 790 - DNA, 21 bp, Artificial Sequence
  siRNA with 19 paired ribonucleotides and two unpaired nucleotides(“t”) on the 3′ end of each strand used in screens of DNA damaging agents camptothecin screen
aggaugaugaugcaguucctt

SEQ ID NO: 791 - DNA, 21 bp, Artificial Sequence
  siRNA with 19 paired ribonucleotides and two unpaired nucleotides(“t”) on the 3′ end of each strand used in screens of DNA damaging agents camptothecin screen
gacaaggagaaugugcgcutt

SEQ ID NO: 792 - DNA, 21 bp, Artificial Sequence
  siRNA with 19 paired ribonucleotides and two unpaired nucleotides(“t”) on the 3′ end of each strand used in screens of DNA damaging agents camptothecin screen
gagcccagucuguugaguutt

SEQ ID NO: 793 - DNA, 21 bp, Artificial Sequence
  siRNA with 19 paired ribonucleotides and two unpaired nucleotides(“t”) on the 3′ end of each strand used in screens of DNA damaging agents camptothecin screen
acguggacgccuccgugautt

SEQ ID NO: 794 - DNA, 21 bp, Artificial Sequence
  siRNA with 19 paired ribonucleotides and two unpaired nucleotides(“t”) on the 3′ end of each strand used in screens of DNA damaging agents camptothecin screen
caccuacuacgagggcggctt

SEQ ID NO: 795 - DNA, 21 bp, Artificial Sequence
  siRNA with 19 paired ribonucleotides and two unpaired nucleotides(“t”) on the 3′ end of each strand used in screens of DNA damaging agents camptothecin screen
caucuacgagacgggggactt

SEQ ID NO: 796 - DNA, 21 bp, Artificial Sequence
  siRNA with 19 paired ribonucleotides and two unpaired nucleotides(“t”) on the 3′ end of each strand used in screens of DNA damaging agents camptothecin screen
cgcauggagcaguuccagatt

SEQ ID NO: 797 - DNA, 21 bp, Artificial Sequence
  siRNA with 19 paired ribonucleotides and two unpaired nucleotides(“t”) on the 3′ end of each strand used in screens of DNA damaging agents camptothecin screen
gacggcuucagcaagagcatt

SEQ ID NO: 798 - DNA, 21 bp, Artificial Sequence
  siRNA with 19 paired ribonucleotides and two unpaired nucleotides(“t”) on the 3′ end of each strand used in screens of DNA damaging agents camptothecin screen
gauuaagacagccgaucgctt

SEQ ID NO: 799 - DNA, 21 bp, Artificial Sequence
  siRNA with 19 paired ribonucleotides and two unpaired nucleotides(“t”) on the 3′ end of each strand used in screens of DNA damaging agents camptothecin screen
accuuauggaaaaggggugtt

SEQ ID NO: 800 - DNA, 21 bp, Artificial Sequence
  siRNA with 19 paired ribonucleotides and two unpaired nucleotides(“t”) on the 3′ end of each strand used in screens of DNA damaging agents camptothecin screen
ccauccuguuugaaagccutt

SEQ ID NO: 801 - DNA, 21 bp, Artificial Sequence
  siRNA with 19 paired ribonucleotides and two unpaired nucleotides(“t”) on the 3′ end of each strand used in screens of DNA damaging agents camptothecin screen
cccaaaaggaagugcuguatt

SEQ ID NO: 802 - DNA, 21 bp, Artificial Sequence
  siRNA with 19 paired ribonucleotides and two unpaired nucleotides(“t”) on the 3′ end of each strand used in screens of DNA damaging agents camptothecin screen
caccugcucaaguccuuugtt

SEQ ID NO: 803 - DNA, 21 bp, Artificial Sequence
  siRNA with 19 paired ribonucleotides and two unpaired nucleotides(“t”) on the 3′ end of each strand used in screens of DNA damaging agents camptothecin screen
gauccuucaggccuuguuctt

SEQ ID NO: 804 - DNA, 21 bp, Artificial Sequence
  siRNA with 19 paired ribonucleotides and two unpaired nucleotides(“t”) on the 3′ end of each strand used in screens of DNA damaging agents camptothecin screen
ugacagugaugggucagagtt

SEQ ID NO: 805 - DNA, 21 bp, Artificial Sequence
  siRNA with 19 paired ribonucleotides and two unpaired nucleotides(“t”) on the 3′ end of each strand used in screens of DNA damaging agents camptothecin screen
augauagcgggggcuaagutt

SEQ ID NO: 806 - DNA, 21 bp, Artificial Sequence
  siRNA with 19 paired ribonucleotides and two unpaired nucleotides(“t”) on the 3′ end of each strand used in screens of DNA damaging agents camptothecin screen
gagcuaucuguuccagcugtt

SEQ ID NO: 807 - DNA, 21 bp, Artificial Sequence
  siRNA with 19 paired ribonucleotides and two unpaired nucleotides(“t”) on the 3′ end of each strand used in screens of DNA damaging agents camptothecin screen
ucuauugcuucaccauggctt

SEQ ID NO: 808 - DNA, 21 bp, Artificial Sequence
  siRNA with 19 paired ribonucleotides and two unpaired nucleotides(“t”) on the 3′ end of each strand used in screens of DNA damaging agents camptothecin screen
agcaaauacgcggagcugctt

SEQ ID NO: 809 - DNA, 21 bp, Artificial Sequence
  siRNA with 19 paired ribonucleotides and two unpaired nucleotides(“t”) on the 3′ end of each strand used in screens of DNA damaging agents camptothecin screen
cugcccagguuuuuuuugutt

SEQ ID NO: 810 - DNA, 21 bp, Artificial Sequence
  siRNA with 19 paired ribonucleotides and two unpaired nucleotides(“t”) on the 3′ end of each strand used in screens of DNA damaging agents camptothecin screen
guuacaguucaucuccccutt

SEQ ID NO: 811 - DNA, 21 bp, Artificial Sequence
  siRNA with 19 paired ribonucleotides and two unpaired nucleotides(“t”) on the 3′ end of each strand used in screens of DNA damaging agents camptothecin screen
cccugguguuugagcaugutt

SEQ ID NO: 812 - DNA, 21 bp, Artificial Sequence
  siRNA with 19 paired ribonucleotides and two unpaired nucleotides(“t”) on the 3′ end of each strand used in screens of DNA damaging agents camptothecin screen
cugaccgggagaucaaggutt

SEQ ID NO: 813 - DNA, 21 bp, Artificial Sequence
  siRNA with 19 paired ribonucleotides and two unpaired nucleotides(“t”) on the 3′ end of each strand used in screens of DNA damaging agents camptothecin screen
gagugugagaguccccaautt

SEQ ID NO: 814 - DNA, 21 bp, Artificial Sequence
  siRNA with 19 paired ribonucleotides and two unpaired nucleotides(“t”) on the 3′ end of each strand used in screens of DNA damaging agents camptothecin screen
aggcgagagccgacucaagtt

SEQ ID NO: 815 - DNA, 21 bp, Artificial Sequence
  siRNA with 19 paired ribonucleotides and two unpaired nucleotides(“t”) on the 3′ end of each strand used in screens of DNA damaging agents camptothecin screen
ccuggaccgcuagggauactt

SEQ ID NO: 816 - DNA, 21 bp, Artificial Sequence
  siRNA with 19 paired ribonucleotides and two unpaired nucleotides(“t”) on the 3′ end of each strand used in screens of DNA damaging agents camptothecin screen
cgcaaccgcgagaaccuuctt

SEQ ID NO: 817 - DNA, 21 bp, Artificial Sequence
  siRNA with 19 paired ribonucleotides and two unpaired nucleotides(“t”) on the 3′ end of each strand used in screens of DNA damaging agents camptothecin screen
aacuggcagauuuuggccutt

SEQ ID NO: 818 - DNA, 21 bp, Artificial Sequence
  siRNA with 19 paired ribonucleotides and two unpaired nucleotides(“t”) on the 3′ end of each strand used in screens of DNA damaging agents camptothecin screen
cuguccaguggaaaccuuatt

SEQ ID NO: 819 - DNA, 21 bp, Artificial Sequence
  siRNA with 19 paired ribonucleotides and two unpaired nucleotides(“t”) on the 3′ end of each strand used in screens of DNA damaging agents camptothecin screen
uagaaccgccuuaagagagtt

SEQ ID NO: 820 - DNA, 21 bp, Artificial Sequence
  siRNA with 19 paired ribonucleotides and two unpaired nucleotides(“t”) on the 3′ end of each strand used in screens of DNA damaging agents camptothecin screen
acaguacccaauuccgacatt

SEQ ID NO: 821 - DNA, 21 bp, Artificial Sequence
  siRNA with 19 paired ribonucleotides and two unpaired nucleotides(“t”) on the 3′ end of each strand used in screens of DNA damaging agents camptothecin screen
ggagaauacuucugcugugtt

SEQ ID NO: 822 - DNA, 21 bp, Artificial Sequence
  siRNA with 19 paired ribonucleotides and two unpaired nucleotides(“t”) on the 3′ end of each strand used in screens of DNA damaging agents camptothecin screen
ucagccacaaugauguccutt

SEQ ID NO: 823 - DNA, 21 bp, Artificial Sequence
  siRNA with 19 paired ribonucleotides and two unpaired nucleotides(“t”) on the 3′ end of each strand used in screens of DNA damaging agents camptothecin screen
aacuaggcgguugaaugagtt

SEQ ID NO: 824 - DNA, 21 bp, Artificial Sequence
  siRNA with 19 paired ribonucleotides and two unpaired nucleotides(“t”) on the 3′ end of each strand used in screens of DNA damaging agents camptothecin screen
cauacuggccuggacuguutt

SEQ ID NO: 825 - DNA, 21 bp, Artificial Sequence
  siRNA with 19 paired ribonucleotides and two unpaired nucleotides(“t”) on the 3′ end of each strand used in screens of DNA damaging agents camptothecin screen
gaugguggcaguagaggcutt

SEQ ID NO: 826 - DNA, 21 bp, Artificial Sequence
  siRNA with 19 paired ribonucleotides and two unpaired nucleotides(“t”) on the 3′ end of each strand used in screens of DNA damaging agents camptothecin screen
aucgauucugcuccucuagtt

SEQ ID NO: 827 - DNA, 21 bp, Artificial Sequence
  siRNA with 19 paired ribonucleotides and two unpaired nucleotides(“t”) on the 3′ end of each strand used in screens of DNA damaging agents camptothecin screen
cugaagaagcagucgcagutt

SEQ ID NO: 828 - DNA, 21 bp, Artificial Sequence
  siRNA with 19 paired ribonucleotides and two unpaired nucleotides(“t”) on the 3′ end of each strand used in screens of DNA damaging agents camptothecin screen
ugccugaaagagacuugugtt

SEQ ID NO: 829 - DNA, 21 bp, Artificial Sequence
  siRNA with 19 paired ribonucleotides and two unpaired nucleotides(“t”) on the 3′ end of each strand used in screens of DNA damaging agents camptothecin screen
ccaguugauguuugguccutt

SEQ ID NO: 830 - DNA, 21 bp, Artificial Sequence
  siRNA with 19 paired ribonucleotides and two unpaired nucleotides(“t”) on the 3′ end of each strand used in screens of DNA damaging agents camptothecin screen
ucucagacuuuggcuuggctt

SEQ ID NO: 831 - DNA, 21 bp, Artificial Sequence
  siRNA with 19 paired ribonucleotides and two unpaired nucleotides(“t”) on the 3′ end of each strand used in screens of DNA damaging agents camptothecin screen
uucuauggucacaggagagtt

SEQ ID NO: 832 - DNA, 21 bp, Artificial Sequence
  siRNA with 19 paired ribonucleotides and two unpaired nucleotides(“t”) on the 3′ end of each strand used in screens of DNA damaging agents camptothecin screen
agacugcguccuuuucguctt

SEQ ID NO: 833 - DNA, 21 bp, Artificial Sequence
  siRNA with 19 paired ribonucleotides and two unpaired nucleotides(“t”) on the 3′ end of each strand used in screens of DNA damaging agents camptothecin screen
gauaccagcaccaguggaatt

SEQ ID NO: 834 - DNA, 21 bp, Artificial Sequence
  siRNA with 19 paired ribonucleotides and two unpaired nucleotides(“t”) on the 3′ end of each strand used in screens of DNA damaging agents camptothecin screen
gcauaccucauccagcauctt

SEQ ID NO: 835 - DNA, 21 bp, Artificial Sequence
  siRNA with 19 paired ribonucleotides and two unpaired nucleotides(“t”) on the 3′ end of each strand used in screens of DNA damaging agents camptothecin screen
ccaaucacaaguccuauugtt

SEQ ID NO: 836 - DNA, 21 bp, Artificial Sequence
  siRNA with 19 paired ribonucleotides and two unpaired nucleotides(“t”) on the 3′ end of each strand used in screens of DNA damaging agents camptothecin screen
cuugugcgaccuccuauuatt

SEQ ID NO: 837 - DNA, 21 bp, Artificial Sequence
  siRNA with 19 paired ribonucleotides and two unpaired nucleotides(“t”) on the 3′ end of each strand used in screens of DNA damaging agents camptothecin screen
gagagaaaagcucgucugatt

SEQ ID NO: 838 - DNA, 21 bp, Artificial Sequence
  siRNA with 19 paired ribonucleotides and two unpaired nucleotides(“t”) on the 3′ end of each strand used in screens of DNA damaging agents camptothecin screen
auuuuugcggcgccagaautt

SEQ ID NO: 839 - DNA, 21 bp, Artificial Sequence
  siRNA with 19 paired ribonucleotides and two unpaired nucleotides(“t”) on the 3′ end of each strand used in screens of DNA damaging agents camptothecin screen
gaaaaacggaggucgcguutt

SEQ ID NO: 840 - DNA, 21 bp, Artificial Sequence
  siRNA with 19 paired ribonucleotides and two unpaired nucleotides(“t”) on the 3′ end of each strand used in screens of DNA damaging agents camptothecin screen
gaaaacaaaugccccgugctt

SEQ ID NO: 841 - DNA, 21 bp, Artificial Sequence
  siRNA with 19 paired ribonucleotides and two unpaired nucleotides(“t”) on the 3′ end of each strand used in screens of DNA damaging agents camptothecin screen
agugcagaaagucaucccatt

SEQ ID NO: 842 - DNA, 21 bp, Artificial Sequence
  siRNA with 19 paired ribonucleotides and two unpaired nucleotides(“t”) on the 3′ end of each strand used in screens of DNA damaging agents camptothecin screen
caaccugcaguuugguaagtt

SEQ ID NO: 843 - DNA, 21 bp, Artificial Sequence
  siRNA with 19 paired ribonucleotides and two unpaired nucleotides(“t”) on the 3′ end of each strand used in screens of DNA damaging agents camptothecin screen
ugagccaaguggcagcuaatt

SEQ ID NO: 844 - DNA, 21 bp, Artificial Sequence
  siRNA with 19 paired ribonucleotides and two unpaired nucleotides(“t”) on the 3′ end of each strand used in screens of DNA damaging agents camptothecin screen
cguuccgauccucuauacutt

SEQ ID NO: 845 - DNA, 21 bp, Artificial Sequence
  siRNA with 19 paired ribonucleotides and two unpaired nucleotides(“t”) on the 3′ end of each strand used in screens of DNA damaging agents camptothecin screen
ugacaucauugugcuggcctt

SEQ ID NO: 846 - DNA, 21 bp, Artificial Sequence
  siRNA with 19 paired ribonucleotides and two unpaired nucleotides(“t”) on the 3′ end of each strand used in screens of DNA damaging agents camptothecin screen
ugaccaaagaugaccugugtt

SEQ ID NO: 847 - DNA, 21 bp, Artificial Sequence
  siRNA with 19 paired ribonucleotides and two unpaired nucleotides(“t”) on the 3′ end of each strand used in screens of DNA damaging agents camptothecin screen
aaguguuguugcuggcagatt

SEQ ID NO: 848 - DNA, 21 bp, Artificial Sequence
  siRNA with 19 paired ribonucleotides and two unpaired nucleotides(“t”) on the 3′ end of each strand used in screens of DNA damaging agents camptothecin screen
acuugagaagcuuuuggggtt

SEQ ID NO: 849 - DNA, 21 bp, Artificial Sequence
  siRNA with 19 paired ribonucleotides and two unpaired nucleotides(“t”) on the 3′ end of each strand used in screens of DNA damaging agents camptothecin screen
cuagagguuuuugcugcagtt

SEQ ID NO: 850 - DNA, 21 bp, Artificial Sequence
  siRNA with 19 paired ribonucleotides and two unpaired nucleotides(“t”) on the 3′ end of each strand used in screens of DNA damaging agents camptothecin screen
aaauuugcgccucgguauctt

SEQ ID NO: 851 - DNA, 21 bp, Artificial Sequence
  siRNA with 19 paired ribonucleotides and two unpaired nucleotides(“t”) on the 3′ end of each strand used in screens of DNA damaging agents camptothecin screen
accuaaguccuuccaccugtt

SEQ ID NO: 852 - DNA, 21 bp, Artificial Sequence
  siRNA with 19 paired ribonucleotides and two unpaired nucleotides(“t”) on the 3′ end of each strand used in screens of DNA damaging agents camptothecin screen
cacccuggaugcuguugaatt

SEQ ID NO: 853 - DNA, 21 bp, Artificial Sequence
  siRNA with 19 paired ribonucleotides and two unpaired nucleotides(“t”) on the 3′ end of each strand used in screens of DNA damaging agents camptothecin screen
gaccgcaaacuacugauuctt

SEQ ID NO: 854 - DNA, 21 bp, Artificial Sequence
  siRNA with 19 paired ribonucleotides and two unpaired nucleotides(“t”) on the 3′ end of each strand used in screens of DNA damaging agents camptothecin screen
gccagcaugaucuccaagutt

SEQ ID NO: 855 - DNA, 21 bp, Artificial Sequence
  siRNA with 19 paired ribonucleotides and two unpaired nucleotides(“t”) on the 3′ end of each strand used in screens of DNA damaging agents camptothecin screen
uagacauuggguucgccgutt

SEQ ID NO: 856 - DNA, 21 bp, Artificial Sequence
  siRNA with 19 paired ribonucleotides and two unpaired nucleotides(“t”) on the 3′ end of each strand used in screens of DNA damaging agents camptothecin screen
gaauauuuuugggacgccgtt

SEQ ID NO: 857 - DNA, 21 bp, Artificial Sequence
  siRNA with 19 paired ribonucleotides and two unpaired nucleotides(“t”) on the 3′ end of each strand used in screens of DNA damaging agents camptothecin screen
uccaagaggcucucagacatt

SEQ ID NO: 858 - DNA, 21 bp, Artificial Sequence
  siRNA with 19 paired ribonucleotides and two unpaired nucleotides(“t”) on the 3′ end of each strand used in screens of DNA damaging agents camptothecin screen
ucucagaagguccuccugatt

SEQ ID NO: 859 - DNA, 21 bp, Artificial Sequence
  siRNA with 19 paired ribonucleotides and two unpaired nucleotides(“t”) on the 3′ end of each strand used in screens of DNA damaging agents camptothecin screen
acaucguggugcgagguuatt

SEQ ID NO: 860 - DNA, 21 bp, Artificial Sequence
  siRNA with 19 paired ribonucleotides and two unpaired nucleotides(“t”) on the 3′ end of each strand used in screens of DNA damaging agents camptothecin screen
augagccgccaauuggacatt

SEQ ID NO: 861 - DNA, 21 bp, Artificial Sequence
  siRNA with 19 paired ribonucleotides and two unpaired nucleotides(“t”) on the 3′ end of each strand used in screens of DNA damaging agents camptothecin screen
auggcaaguuuggaaggactt

SEQ ID NO: 862 - DNA, 21 bp, Artificial Sequence
  siRNA with 19 paired ribonucleotides and two unpaired nucleotides(“t”) on the 3′ end of each strand used in screens of DNA damaging agents camptothecin screen
aacaagaggacacaauggctt

SEQ ID NO: 863 - DNA, 21 bp, Artificial Sequence
  siRNA with 19 paired ribonucleotides and two unpaired nucleotides(“t”) on the 3′ end of each strand used in screens of DNA damaging agents camptothecin screen
agaggugaagaucaugucgtt

SEQ ID NO: 864 - DNA, 21 bp, Artificial Sequence
  siRNA with 19 paired ribonucleotides and two unpaired nucleotides(“t”) on the 3′ end of each strand used in screens of DNA damaging agents camptothecin screen
ucgcagacuuuggcaugagtt

SEQ ID NO: 865 - DNA, 21 bp, Artificial Sequence
  siRNA with 19 paired ribonucleotides and two unpaired nucleotides(“t”) on the 3′ end of each strand used in screens of DNA damaging agents camptothecin screen
caagugggacaugcugaagtt

SEQ ID NO: 866 - DNA, 21 bp, Artificial Sequence
  siRNA with 19 paired ribonucleotides and two unpaired nucleotides(“t”) on the 3′ end of each strand used in screens of DNA damaging agents camptothecin screen
uaaaaggcccuccaucugctt

SEQ ID NO: 867 - DNA, 21 bp, Artificial Sequence
  siRNA with 19 paired ribonucleotides and two unpaired nucleotides(“t”) on the 3′ end of each strand used in screens of DNA damaging agents camptothecin screen
uuggcccuguucagcaaugtt

SEQ ID NO: 868 - DNA, 21 bp, Artificial Sequence
  siRNA with 19 paired ribonucleotides and two unpaired nucleotides(“t”) on the 3′ end of each strand used in screens of DNA damaging agents camptothecin screen
aacccaccugaugaggacutt

SEQ ID NO: 869 - DNA, 21 bp, Artificial Sequence
  siRNA with 19 paired ribonucleotides and two unpaired nucleotides(“t”) on the 3′ end of each strand used in screens of DNA damaging agents camptothecin screen
gaccgaguuuggauccaactt

SEQ ID NO: 870 - DNA, 21 bp, Artificial Sequence
  siRNA with 19 paired ribonucleotides and two unpaired nucleotides(“t”) on the 3′ end of each strand used in screens of DNA damaging agents camptothecin screen
gauggaguuccaccucauctt

SEQ ID NO: 871 - DNA, 21 bp, Artificial Sequence
  siRNA with 19 paired ribonucleotides and two unpaired nucleotides(“t”) on the 3′ end of each strand used in screens of DNA damaging agents camptothecin screen
ccauguuugcauggccuuutt

SEQ ID NO: 872 - DNA, 21 bp, Artificial Sequence
  siRNA with 19 paired ribonucleotides and two unpaired nucleotides(“t”) on the 3′ end of each strand used in screens of DNA damaging agents camptothecin screen
cuucuggagcaauccaaactt

SEQ ID NO: 873 - DNA, 21 bp, Artificial Sequence
  siRNA with 19 paired ribonucleotides and two unpaired nucleotides(“t”) on the 3′ end of each strand used in screens of DNA damaging agents camptothecin screen
ucuuuggaugcccuccacatt

SEQ ID NO: 874 - DNA, 21 bp, Artificial Sequence
  siRNA with 19 paired ribonucleotides and two unpaired nucleotides(“t”) on the 3′ end of each strand used in screens of DNA damaging agents camptothecin screen
acuggcuaaagaugcugugtt

SEQ ID NO: 875 - DNA, 21 bp, Artificial Sequence
  siRNA with 19 paired ribonucleotides and two unpaired nucleotides(“t”) on the 3′ end of each strand used in screens of DNA damaging agents camptothecin screen
gaccaugggaaaauuguggtt

SEQ ID NO: 876 - DNA, 21 bp, Artificial Sequence
  siRNA with 19 paired ribonucleotides and two unpaired nucleotides(“t”) on the 3′ end of each strand used in screens of DNA damaging agents camptothecin screen
gcuuaguacagcggguugatt

SEQ ID NO: 877 - DNA, 21 bp, Artificial Sequence
  siRNA with 19 paired ribonucleotides and two unpaired nucleotides(“t”) on the 3′ end of each strand used in screens of DNA damaging agents camptothecin screen
cacuaccacccuuuccugutt

SEQ ID NO: 878 - DNA, 21 bp, Artificial Sequence
  siRNA with 19 paired ribonucleotides and two unpaired nucleotides(“t”) on the 3′ end of each strand used in screens of DNA damaging agents camptothecin screen
gugcaaugugaugggaggatt

SEQ ID NO: 879 - DNA, 21 bp, Artificial Sequence
  siRNA with 19 paired ribonucleotides and two unpaired nucleotides(“t”) on the 3′ end of each strand used in screens of DNA damaging agents camptothecin screen
uguuaccggagcugauuugtt

SEQ ID NO: 880 - DNA, 21 bp, Artificial Sequence
  siRNA with 19 paired ribonucleotides and two unpaired nucleotides(“t”) on the 3′ end of each strand used in screens of DNA damaging agents camptothecin screen
gccauuccuuugucugccatt

SEQ ID NO: 881 - DNA, 21 bp, Artificial Sequence
  siRNA with 19 paired ribonucleotides and two unpaired nucleotides(“t”) on the 3′ end of each strand used in screens of DNA damaging agents camptothecin screen
gugaaaguagaagggcccatt

SEQ ID NO: 882 - DNA, 21 bp, Artificial Sequence
  siRNA with 19 paired ribonucleotides and two unpaired nucleotides(“t”) on the 3′ end of each strand used in screens of DNA damaging agents camptothecin screen
uucaagcugguggaugcagtt

SEQ ID NO: 883 - DNA, 21 bp, Artificial Sequence
  siRNA with 19 paired ribonucleotides and two unpaired nucleotides(“t”) on the 3′ end of each strand used in screens of DNA damaging agents camptothecin screen
aggacccuuucaauguccctt

SEQ ID NO: 884 - DNA, 21 bp, Artificial Sequence
  siRNA with 19 paired ribonucleotides and two unpaired nucleotides(“t”) on the 3′ end of each strand used in screens of DNA damaging agents camptothecin screen
cucucauuaucuccuccactt

SEQ ID NO: 885 - DNA, 21 bp, Artificial Sequence
  siRNA with 19 paired ribonucleotides and two unpaired nucleotides(“t”) on the 3′ end of each strand used in screens of DNA damaging agents camptothecin screen
gcucuccuuccaguuuccatt

SEQ ID NO: 886 - DNA, 21 bp, Artificial Sequence
  siRNA with 19 paired ribonucleotides and two unpaired nucleotides(“t”) on the 3′ end of each strand used in screens of DNA damaging agents camptothecin screen
cuggcuacgaacugauuggtt

SEQ ID NO: 887 - DNA, 21 bp, Artificial Sequence
  siRNA with 19 paired ribonucleotides and two unpaired nucleotides(“t”) on the 3′ end of each strand used in screens of DNA damaging agents camptothecin screen
gauuccuauccgguggacutt

SEQ ID NO: 888 - DNA, 21 bp, Artificial Sequence
  siRNA with 19 paired ribonucleotides and two unpaired nucleotides(“t”) on the 3′ end of each strand used in screens of DNA damaging agents camptothecin screen
gcuaucguauaguucggactt

SEQ ID NO: 889 - DNA, 21 bp, Artificial Sequence
  siRNA with 19 paired ribonucleotides and two unpaired nucleotides(“t”) on the 3′ end of each strand used in screens of DNA damaging agents camptothecin screen
gaagauccugagcaguauctt

SEQ ID NO: 890 - DNA, 21 bp, Artificial Sequence
  siRNA with 19 paired ribonucleotides and two unpaired nucleotides(“t”) on the 3′ end of each strand used in screens of DNA damaging agents camptothecin screen
gcugcagcaguacauugcutt

SEQ ID NO: 891 - DNA, 21 bp, Artificial Sequence
  siRNA with 19 paired ribonucleotides and two unpaired nucleotides(“t”) on the 3′ end of each strand used in screens of DNA damaging agents camptothecin screen
uacccuggacaagcucauctt

SEQ ID NO: 892 - DNA, 21 bp, Artificial Sequence
  siRNA with 19 paired ribonucleotides and two unpaired nucleotides(“t”) on the 3′ end of each strand used in screens of DNA damaging agents camptothecin screen
uucagccugaaagguguagtt

SEQ ID NO: 893 - DNA, 21 bp, Artificial Sequence
  siRNA with 19 paired ribonucleotides and two unpaired nucleotides(“t”) on the 3′ end of each strand used in screens of DNA damaging agents camptothecin screen
acgcuacguguaccgcuuutt

SEQ ID NO: 894 - DNA, 21 bp, Artificial Sequence
  siRNA with 19 paired ribonucleotides and two unpaired nucleotides(“t”) on the 3′ end of each strand used in screens of DNA damaging agents camptothecin screen
ugacuaccccucggucauutt

SEQ ID NO: 895 - DNA, 21 bp, Artificial Sequence
  siRNA with 19 paired ribonucleotides and two unpaired nucleotides(“t”) on the 3′ end of each strand used in screens of DNA damaging agents camptothecin screen
acaucgaaaacagcaggugtt

SEQ ID NO: 896 - DNA, 21 bp, Artificial Sequence
  siRNA with 19 paired ribonucleotides and two unpaired nucleotides(“t”) on the 3′ end of each strand used in screens of DNA damaging agents camptothecin screen
aggaggagugcaucuuuggtt

SEQ ID NO: 897 - DNA, 21 bp, Artificial Sequence
  siRNA with 19 paired ribonucleotides and two unpaired nucleotides(“t”) on the 3′ end of each strand used in screens of DNA damaging agents camptothecin screen
uggaugaggacuuuugcagtt

SEQ ID NO: 898 - DNA, 21 bp, Artificial Sequence
  siRNA with 19 paired ribonucleotides and two unpaired nucleotides(“t”) on the 3′ end of each strand used in screens of DNA damaging agents camptothecin screen
cuaugcguacugcauagcatt

SEQ ID NO: 899 - DNA, 21 bp, Artificial Sequence
  siRNA with 19 paired ribonucleotides and two unpaired nucleotides(“t”) on the 3′ end of each strand used in screens of DNA damaging agents camptothecin screen
gacaacgacacauagcuggtt

SEQ ID NO: 900 - DNA, 21 bp, Artificial Sequence
  siRNA with 19 paired ribonucleotides and two unpaired nucleotides(“t”) on the 3′ end of each strand used in screens of DNA damaging agents camptothecin screen
uacaaggaaccucagagcctt

SEQ ID NO: 901 - DNA, 21 bp, Artificial Sequence
  siRNA with 19 paired ribonucleotides and two unpaired nucleotides(“t”) on the 3′ end of each strand used in screens of DNA damaging agents camptothecin screen
aagcuaaugacgaggaacctt

SEQ ID NO: 902 - DNA, 21 bp, Artificial Sequence
  siRNA with 19 paired ribonucleotides and two unpaired nucleotides(“t”) on the 3′ end of each strand used in screens of DNA damaging agents camptothecin screen
ccauggugaagugcuuggatt

SEQ ID NO: 903 - DNA, 21 bp, Artificial Sequence
  siRNA with 19 paired ribonucleotides and two unpaired nucleotides(“t”) on the 3′ end of each strand used in screens of DNA damaging agents camptothecin screen
uaacagcugcaauucccugtt

SEQ ID NO: 904 - DNA, 21 bp, Artificial Sequence
  siRNA with 19 paired ribonucleotides and two unpaired nucleotides(“t”) on the 3′ end of each strand used in screens of DNA damaging agents camptothecin screen
acuauagacuuccgcaacctt

SEQ ID NO: 905 - DNA, 21 bp, Artificial Sequence
  siRNA with 19 paired ribonucleotides and two unpaired nucleotides(“t”) on the 3′ end of each strand used in screens of DNA damaging agents camptothecin screen
caguagauugcuguggccutt

SEQ ID NO: 906 - DNA, 21 bp, Artificial Sequence
  siRNA with 19 paired ribonucleotides and two unpaired nucleotides(“t”) on the 3′ end of each strand used in screens of DNA damaging agents camptothecin screen
cuccauacagcuucugaagtt

SEQ ID NO: 907 - DNA, 21 bp, Artificial Sequence
  siRNA with 19 paired ribonucleotides and two unpaired nucleotides(“t”) on the 3′ end of each strand used in screens of DNA damaging agents camptothecin screen
gacuuuccagaccuggcagtt

SEQ ID NO: 908 - DNA, 21 bp, Artificial Sequence
  siRNA with 19 paired ribonucleotides and two unpaired nucleotides(“t”) on the 3′ end of each strand used in screens of DNA damaging agents camptothecin screen
gaucggggucuucuccauctt

SEQ ID NO: 909 - DNA, 21 bp, Artificial Sequence
  siRNA with 19 paired ribonucleotides and two unpaired nucleotides(“t”) on the 3′ end of each strand used in screens of DNA damaging agents camptothecin screen
ggacuucgcgcuggucuggtt

SEQ ID NO: 910 - DNA, 21 bp, Artificial Sequence
  siRNA with 19 paired ribonucleotides and two unpaired nucleotides(“t”) on the 3′ end of each strand used in screens of DNA damaging agents camptothecin screen
aagcaagaaauggcggcagtt

SEQ ID NO: 911 - DNA, 21 bp, Artificial Sequence
  siRNA with 19 paired ribonucleotides and two unpaired nucleotides(“t”) on the 3′ end of each strand used in screens of DNA damaging agents camptothecin screen
gagcugaaugucuuugggctt

SEQ ID NO: 912 - DNA, 21 bp, Artificial Sequence
  siRNA with 19 paired ribonucleotides and two unpaired nucleotides(“t”) on the 3′ end of each strand used in screens of DNA damaging agents camptothecin screen
uguauauagcgaccagagctt

SEQ ID NO: 913 - DNA, 21 bp, Artificial Sequence
  siRNA with 19 paired ribonucleotides and two unpaired nucleotides(“t”) on the 3′ end of each strand used in screens of DNA damaging agents camptothecin screen
cggcaaagauuacuacuuutt

SEQ ID NO: 914 - DNA, 21 bp, Artificial Sequence
  siRNA with 19 paired ribonucleotides and two unpaired nucleotides(“t”) on the 3′ end of each strand used in screens of DNA damaging agents camptothecin screen
ggagcccggccuguuugautt

SEQ ID NO: 915 - DNA, 21 bp, Artificial Sequence
  siRNA with 19 paired ribonucleotides and two unpaired nucleotides(“t”) on the 3′ end of each strand used in screens of DNA damaging agents camptothecin screen
ucaagaaagcucaaaggactt

SEQ ID NO: 916 - DNA, 21 bp, Artificial Sequence
  siRNA with 19 paired ribonucleotides and two unpaired nucleotides(“t”) on the 3′ end of each strand used in screens of DNA damaging agents camptothecin screen
cccagagauuuuugagggatt

SEQ ID NO: 917 - DNA, 21 bp, Artificial Sequence
  siRNA with 19 paired ribonucleotides and two unpaired nucleotides(“t”) on the 3′ end of each strand used in screens of DNA damaging agents camptothecin screen
ugccuucaacguaggcgautt

SEQ ID NO: 918 - DNA, 21 bp, Artificial Sequence
  siRNA with 19 paired ribonucleotides and two unpaired nucleotides(“t”) on the 3′ end of each strand used in screens of DNA damaging agents camptothecin screen
ugguaccuauuagggauggtt

SEQ ID NO: 919 - DNA, 21 bp, Artificial Sequence
  siRNA with 19 paired ribonucleotides and two unpaired nucleotides(“t”) on the 3′ end of each strand used in screens of DNA damaging agents camptothecin screen
agaggacguuuucuacggctt

SEQ ID NO: 920 - DNA, 21 bp, Artificial Sequence
  siRNA with 19 paired ribonucleotides and two unpaired nucleotides(“t”) on the 3′ end of each strand used in screens of DNA damaging agents camptothecin screen
aucuguuugcaaggggaagtt

SEQ ID NO: 921 - DNA, 21 bp, Artificial Sequence
  siRNA with 19 paired ribonucleotides and two unpaired nucleotides(“t”) on the 3′ end of each strand used in screens of DNA damaging agents camptothecin screen
caagaucauccccaagccatt

SEQ ID NO: 922 - DNA, 21 bp, Artificial Sequence
  siRNA with 19 paired ribonucleotides and two unpaired nucleotides(“t”) on the 3′ end of each strand used in screens of DNA damaging agents camptothecin screen
aaacuguucucagaugccctt

SEQ ID NO: 923 - DNA, 21 bp, Artificial Sequence
  siRNA with 19 paired ribonucleotides and two unpaired nucleotides(“t”) on the 3′ end of each strand used in screens of DNA damaging agents camptothecin screen
cccaacuugaaagugcguutt

SEQ ID NO: 924 - DNA, 21 bp, Artificial Sequence
  siRNA with 19 paired ribonucleotides and two unpaired nucleotides(“t”) on the 3′ end of each strand used in screens of DNA damaging agents camptothecin screen
ugagaugcacuccuccagutt

SEQ ID NO: 925 - DNA, 21 bp, Artificial Sequence
  siRNA with 19 paired ribonucleotides and two unpaired nucleotides(“t”) on the 3′ end of each strand used in screens of DNA damaging agents camptothecin screen
aagcuucuuguagcuggugtt

SEQ ID NO: 926 - DNA, 21 bp, Artificial Sequence
  siRNA with 19 paired ribonucleotides and two unpaired nucleotides(“t”) on the 3′ end of each strand used in screens of DNA damaging agents camptothecin screen
auauugccuggacagguggtt

SEQ ID NO: 927 - DNA, 21 bp, Artificial Sequence
  siRNA with 19 paired ribonucleotides and two unpaired nucleotides(“t”) on the 3′ end of each strand used in screens of DNA damaging agents camptothecin screen
cagcaacaagaacuccuagtt

SEQ ID NO: 928 - DNA, 21 bp, Artificial Sequence
  siRNA with 19 paired ribonucleotides and two unpaired nucleotides(“t”) on the 3′ end of each strand used in screens of DNA damaging agents camptothecin screen
agcauucauggaggcucuutt

SEQ ID NO: 929 - DNA, 21 bp, Artificial Sequence
  siRNA with 19 paired ribonucleotides and two unpaired nucleotides(“t”) on the 3′ end of each strand used in screens of DNA damaging agents camptothecin screen
auugacaucauccccaacctt

SEQ ID NO: 930 - DNA, 21 bp, Artificial Sequence
  siRNA with 19 paired ribonucleotides and two unpaired nucleotides(“t”) on the 3′ end of each strand used in screens of DNA damaging agents camptothecin screen
cucagcuuuuguggagcgatt

SEQ ID NO: 931 - DNA, 21 bp, Artificial Sequence
  siRNA with 19 paired ribonucleotides and two unpaired nucleotides(“t”) on the 3′ end of each strand used in screens of DNA damaging agents camptothecin screen
agggcaguggagacuauautt

SEQ ID NO: 932 - DNA, 21 bp, Artificial Sequence
  siRNA with 19 paired ribonucleotides and two unpaired nucleotides(“t”) on the 3′ end of each strand used in screens of DNA damaging agents camptothecin screen
ccagagugccaaagugauctt

SEQ ID NO: 933 - DNA, 21 bp, Artificial Sequence
  siRNA with 19 paired ribonucleotides and two unpaired nucleotides(“t”) on the 3′ end of each strand used in screens of DNA damaging agents camptothecin screen
ggauauauuuggcugggugtt

SEQ ID NO: 934 - DNA, 21 bp, Artificial Sequence
  siRNA with 19 paired ribonucleotides and two unpaired nucleotides(“t”) on the 3′ end of each strand used in screens of DNA damaging agents camptothecin screen
gauaacggcuucuggaagatt

SEQ ID NO: 935 - DNA, 21 bp, Artificial Sequence
  siRNA with 19 paired ribonucleotides and two unpaired nucleotides(“t”) on the 3′ end of each strand used in screens of DNA damaging agents camptothecin screen
gcaaaagcaacuuuggcagtt

SEQ ID NO: 936 - DNA, 21 bp, Artificial Sequence
  siRNA with 19 paired ribonucleotides and two unpaired nucleotides(“t”) on the 3′ end of each strand used in screens of DNA damaging agents camptothecin screen
gcucaauaucagcugggaatt

SEQ ID NO: 937 - DNA, 21 bp, Artificial Sequence
  siRNA with 19 paired ribonucleotides and two unpaired nucleotides(“t”) on the 3′ end of each strand used in screens of DNA damaging agents camptothecin screen
gagcuauaccaugcugggatt

SEQ ID NO: 938 - DNA, 21 bp, Artificial Sequence
  siRNA with 19 paired ribonucleotides and two unpaired nucleotides(“t”) on the 3′ end of each strand used in screens of DNA damaging agents camptothecin screen
ggauggacggacagagguutt

SEQ ID NO: 939 - DNA, 21 bp, Artificial Sequence
  siRNA with 19 paired ribonucleotides and two unpaired nucleotides(“t”) on the 3′ end of each strand used in screens of DNA damaging agents camptothecin screen
guuacuggugauucucugctt

SEQ ID NO: 940 - DNA, 21 bp, Artificial Sequence
  siRNA with 19 paired ribonucleotides and two unpaired nucleotides(“t”) on the 3′ end of each strand used in screens of DNA damaging agents camptothecin screen
augcggaauuugccgugggtt

SEQ ID NO: 941 - DNA, 21 bp, Artificial Sequence
  siRNA with 19 paired ribonucleotides and two unpaired nucleotides(“t”) on the 3′ end of each strand used in screens of DNA damaging agents camptothecin screen
gcacaagcaccugugugagtt

SEQ ID NO: 942 - DNA, 21 bp, Artificial Sequence
  siRNA with 19 paired ribonucleotides and two unpaired nucleotides(“t”) on the 3′ end of each strand used in screens of DNA damaging agents camptothecin screen
gucguacaccaugaucgggtt

SEQ ID NO: 943 - DNA, 21 bp, Artificial Sequence
  siRNA with 19 paired ribonucleotides and two unpaired nucleotides(“t”) on the 3′ end of each strand used in screens of DNA damaging agents camptothecin screen
acaaaaacggagauccguctt

SEQ ID NO: 944 - DNA, 21 bp, Artificial Sequence
  siRNA with 19 paired ribonucleotides and two unpaired nucleotides(“t”) on the 3′ end of each strand used in screens of DNA damaging agents camptothecin screen
auaagcagcaagaaacggctt

SEQ ID NO: 945 - DNA, 21 bp, Artificial Sequence
  siRNA with 19 paired ribonucleotides and two unpaired nucleotides(“t”) on the 3′ end of each strand used in screens of DNA damaging agents camptothecin screen
gaauuucgggcuacuuuggtt

SEQ ID NO: 946 - DNA, 21 bp, Artificial Sequence
  siRNA with 19 paired ribonucleotides and two unpaired nucleotides(“t”) on the 3′ end of each strand used in screens of DNA damaging agents camptothecin screen
aaacgguccauugguaggatt

SEQ ID NO: 947 - DNA, 21 bp, Artificial Sequence
  siRNA with 19 paired ribonucleotides and two unpaired nucleotides(“t”) on the 3′ end of each strand used in screens of DNA damaging agents camptothecin screen
aaguggaaggaagucgggatt

SEQ ID NO: 948 - DNA, 21 bp, Artificial Sequence
  siRNA with 19 paired ribonucleotides and two unpaired nucleotides(“t”) on the 3′ end of each strand used in screens of DNA damaging agents camptothecin screen
ugccaagcaguuugaacugtt

SEQ ID NO: 949 - DNA, 21 bp, Artificial Sequence
  siRNA with 19 paired ribonucleotides and two unpaired nucleotides(“t”) on the 3′ end of each strand used in screens of DNA damaging agents camptothecin screen
ccugcagcaacugauuacctt

SEQ ID NO: 950 - DNA, 21 bp, Artificial Sequence
  siRNA with 19 paired ribonucleotides and two unpaired nucleotides(“t”) on the 3′ end of each strand used in screens of DNA damaging agents camptothecin screen
gaacuugagaagaugcgagtt

SEQ ID NO: 951 - DNA, 21 bp, Artificial Sequence
  siRNA with 19 paired ribonucleotides and two unpaired nucleotides(“t”) on the 3′ end of each strand used in screens of DNA damaging agents camptothecin screen
gaagaggcccacugaaguutt

SEQ ID NO: 952 - DNA, 21 bp, Artificial Sequence
  siRNA with 19 paired ribonucleotides and two unpaired nucleotides(“t”) on the 3′ end of each strand used in screens of DNA damaging agents camptothecin screen
gaaaagacuccuggcugugtt

SEQ ID NO: 953 - DNA, 21 bp, Artificial Sequence
  siRNA with 19 paired ribonucleotides and two unpaired nucleotides(“t”) on the 3′ end of each strand used in screens of DNA damaging agents camptothecin screen
ggacucugaagauguaccutt

SEQ ID NO: 954 - DNA, 21 bp, Artificial Sequence
  siRNA with 19 paired ribonucleotides and two unpaired nucleotides(“t”) on the 3′ end of each strand used in screens of DNA damaging agents camptothecin screen
ggcauacuaguacaaguggtt

SEQ ID NO: 955 - DNA, 21 bp, Artificial Sequence
  siRNA with 19 paired ribonucleotides and two unpaired nucleotides(“t”) on the 3′ end of each strand used in screens of DNA damaging agents camptothecin screen
aacagccauccaagucuuctt

SEQ ID NO: 956 - DNA, 21 bp, Artificial Sequence
  siRNA with 19 paired ribonucleotides and two unpaired nucleotides(“t”) on the 3′ end of each strand used in screens of DNA damaging agents camptothecin screen
aaccuaccagcagaagguutt

SEQ ID NO: 957 - DNA, 21 bp, Artificial Sequence
  siRNA with 19 paired ribonucleotides and two unpaired nucleotides(“t”) on the 3′ end of each strand used in screens of DNA damaging agents camptothecin screen
uaggcuuuucaggaccuuctt

SEQ ID NO: 958 - DNA, 21 bp, Artificial Sequence
  siRNA with 19 paired ribonucleotides and two unpaired nucleotides(“t”) on the 3′ end of each strand used in screens of DNA damaging agents camptothecin screen
aguccuacaggaagagccctt

SEQ ID NO: 959 - DNA, 21 bp, Artificial Sequence
  siRNA with 19 paired ribonucleotides and two unpaired nucleotides(“t”) on the 3′ end of each strand used in screens of DNA damaging agents camptothecin screen
gcuacuugaacacagcuuctt

SEQ ID NO: 960 - DNA, 21 bp, Artificial Sequence
  siRNA with 19 paired ribonucleotides and two unpaired nucleotides(“t”) on the 3′ end of each strand used in screens of DNA damaging agents camptothecin screen
ucaacgaccuggagaacuutt

SEQ ID NO: 961 - DNA, 21 bp, Artificial Sequence
  siRNA with 19 paired ribonucleotides and two unpaired nucleotides(“t”) on the 3′ end of each strand used in screens of DNA damaging agents camptothecin screen
ucacuuagcagcugagucutt

SEQ ID NO: 962 - DNA, 21 bp, Artificial Sequence
  siRNA with 19 paired ribonucleotides and two unpaired nucleotides(“t”) on the 3′ end of each strand used in screens of DNA damaging agents camptothecin screen
uugacagcacuggucagagtt

SEQ ID NO: 963 - DNA, 21 bp, Artificial Sequence
  siRNA with 19 paired ribonucleotides and two unpaired nucleotides(“t”) on the 3′ end of each strand used in screens of DNA damaging agents camptothecin screen
uuggcaagaacuucuuggctt

SEQ ID NO: 964 - DNA, 21 bp, Artificial Sequence
  siRNA with 19 paired ribonucleotides and two unpaired nucleotides(“t”) on the 3′ end of each strand used in screens of DNA damaging agents camptothecin screen
agaacgaucguccaguggatt

SEQ ID NO: 965 - DNA, 21 bp, Artificial Sequence
  siRNA with 19 paired ribonucleotides and two unpaired nucleotides(“t”) on the 3′ end of each strand used in screens of DNA damaging agents camptothecin screen
gguaccucgaugccauagutt

SEQ ID NO: 966 - DNA, 21 bp, Artificial Sequence
  siRNA with 19 paired ribonucleotides and two unpaired nucleotides(“t”) on the 3′ end of each strand used in screens of DNA damaging agents camptothecin screen
uuuuggacuagugcggaugtt

SEQ ID NO: 967 - DNA, 21 bp, Artificial Sequence
  siRNA with 19 paired ribonucleotides and two unpaired nucleotides(“t”) on the 3′ end of each strand used in screens of DNA damaging agents camptothecin screen
aaggcugccacaaauguugtt

SEQ ID NO: 968 - DNA, 21 bp, Artificial Sequence
  siRNA with 19 paired ribonucleotides and two unpaired nucleotides(“t”) on the 3′ end of each strand used in screens of DNA damaging agents camptothecin screen
gaaacagaagcacgagaugtt

SEQ ID NO: 969 - DNA, 21 bp, Artificial Sequence
  siRNA with 19 paired ribonucleotides and two unpaired nucleotides(“t”) on the 3′ end of each strand used in screens of DNA damaging agents camptothecin screen
ucucuacaucuuggcuggatt

SEQ ID NO: 970 - DNA, 21 bp, Artificial Sequence
  siRNA with 19 paired ribonucleotides and two unpaired nucleotides(“t”) on the 3′ end of each strand used in screens of DNA damaging agents camptothecin screen
cucacaguggaugaauggatt

SEQ ID NO: 971 - DNA, 21 bp, Artificial Sequence
  siRNA with 19 paired ribonucleotides and two unpaired nucleotides(“t”) on the 3′ end of each strand used in screens of DNA damaging agents camptothecin screen
gaucauggggauggaguuctt

SEQ ID NO: 972 - DNA, 21 bp, Artificial Sequence
  siRNA with 19 paired ribonucleotides and two unpaired nucleotides(“t”) on the 3′ end of each strand used in screens of DNA damaging agents camptothecin screen
uacagccuuucaagcagagtt

SEQ ID NO: 973 - DNA, 21 bp, Artificial Sequence
  siRNA with 19 paired ribonucleotides and two unpaired nucleotides(“t”) on the 3′ end of each strand used in screens of DNA damaging agents camptothecin screen
cacccguacaucaaugucutt

SEQ ID NO: 974 - DNA, 21 bp, Artificial Sequence
  siRNA with 19 paired ribonucleotides and two unpaired nucleotides(“t”) on the 3′ end of each strand used in screens of DNA damaging agents camptothecin screen
ggaauaguaugcgcagcuutt

SEQ ID NO: 975 - DNA, 21 bp, Artificial Sequence
  siRNA with 19 paired ribonucleotides and two unpaired nucleotides(“t”) on the 3′ end of each strand used in screens of DNA damaging agents camptothecin screen
gugauucagauggagcuagtt

SEQ ID NO: 976 - DNA, 21 bp, Artificial Sequence
  siRNA with 19 paired ribonucleotides and two unpaired nucleotides(“t”) on the 3′ end of each strand used in screens of DNA damaging agents camptothecin screen
gaguauuaacagccuggactt

SEQ ID NO: 977 - DNA, 21 bp, Artificial Sequence
  siRNA with 19 paired ribonucleotides and two unpaired nucleotides(“t”) on the 3′ end of each strand used in screens of DNA damaging agents camptothecin screen
gcuaagcuagaacacgagutt

SEQ ID NO: 978 - DNA, 21 bp, Artificial Sequence
  siRNA with 19 paired ribonucleotides and two unpaired nucleotides(“t”) on the 3′ end of each strand used in screens of DNA damaging agents camptothecin screen
uagaggauguguuucagcctt

SEQ ID NO: 979 - DNA, 21 bp, Artificial Sequence
  siRNA with 19 paired ribonucleotides and two unpaired nucleotides(“t”) on the 3′ end of each strand used in screens of DNA damaging agents camptothecin screen
cagcuuuguucaggggcagtt

SEQ ID NO: 980 - DNA, 21 bp, Artificial Sequence
  siRNA with 19 paired ribonucleotides and two unpaired nucleotides(“t”) on the 3′ end of each strand used in screens of DNA damaging agents camptothecin screen
cuucgugacaucgagcucatt

SEQ ID NO: 981 - DNA, 21 bp, Artificial Sequence
  siRNA with 19 paired ribonucleotides and two unpaired nucleotides(“t”) on the 3′ end of each strand used in screens of DNA damaging agents camptothecin screen
ggauuacaacccucugcugtt

SEQ ID NO: 982 - DNA, 21 bp, Artificial Sequence
  siRNA with 19 paired ribonucleotides and two unpaired nucleotides(“t”) on the 3′ end of each strand used in screens of DNA damaging agents camptothecin screen
acaacagcacucuucaguctt

SEQ ID NO: 983 - DNA, 21 bp, Artificial Sequence
  siRNA with 19 paired ribonucleotides and two unpaired nucleotides(“t”) on the 3′ end of each strand used in screens of DNA damaging agents camptothecin screen
cugcgagagcgaguuuuactt

SEQ ID NO: 984 - DNA, 21 bp, Artificial Sequence
  siRNA with 19 paired ribonucleotides and two unpaired nucleotides(“t”) on the 3′ end of each strand used in screens of DNA damaging agents camptothecin screen
uguguauucuggagguagctt

SEQ ID NO: 985 - DNA, 21 bp, Artificial Sequence
  siRNA with 19 paired ribonucleotides and two unpaired nucleotides(“t”) on the 3′ end of each strand used in screens of DNA damaging agents camptothecin screen
aguugaggagguuucugcatt

SEQ ID NO: 986 - DNA, 21 bp, Artificial Sequence
  siRNA with 19 paired ribonucleotides and two unpaired nucleotides(“t”) on the 3′ end of each strand used in screens of DNA damaging agents camptothecin screen
gacuuagagcugggaaucutt

SEQ ID NO: 987 - DNA, 21 bp, Artificial Sequence
  siRNA with 19 paired ribonucleotides and two unpaired nucleotides(“t”) on the 3′ end of each strand used in screens of DNA damaging agents camptothecin screen
ggauuauauccagcagcuctt

SEQ ID NO: 988 - DNA, 21 bp, Artificial Sequence
  siRNA with 19 paired ribonucleotides and two unpaired nucleotides(“t”) on the 3′ end of each strand used in screens of DNA damaging agents camptothecin screen
aggauuuuguggccuccautt

SEQ ID NO: 989 - DNA, 21 bp, Artificial Sequence
  siRNA with 19 paired ribonucleotides and two unpaired nucleotides(“t”) on the 3′ end of each strand used in screens of DNA damaging agents camptothecin screen
gucucagcuucugcgguautt

SEQ ID NO: 990 - DNA, 21 bp, Artificial Sequence
  siRNA with 19 paired ribonucleotides and two unpaired nucleotides(“t”) on the 3′ end of each strand used in screens of DNA damaging agents camptothecin screen
uccagguugaaggcauucatt

SEQ ID NO: 991 - DNA, 21 bp, Artificial Sequence
  siRNA with 19 paired ribonucleotides and two unpaired nucleotides(“t”) on the 3′ end of each strand used in screens of DNA damaging agents camptothecin screen
gcagaugagcaaggaugcutt

SEQ ID NO: 992 - DNA, 21 bp, Artificial Sequence
  siRNA with 19 paired ribonucleotides and two unpaired nucleotides(“t”) on the 3′ end of each strand used in screens of DNA damaging agents camptothecin screen
guacauccauguggccaaatt

SEQ ID NO: 993 - DNA, 21 bp, Artificial Sequence
  siRNA with 19 paired ribonucleotides and two unpaired nucleotides(“t”) on the 3′ end of each strand used in screens of DNA damaging agents camptothecin screen
ugggucaugaaagcugccatt

SEQ ID NO: 994 - DNA, 21 bp, Artificial Sequence
  siRNA with 19 paired ribonucleotides and two unpaired nucleotides(“t”) on the 3′ end of each strand used in screens of DNA damaging agents camptothecin screen
aacugaaagccccuccuaatt

SEQ ID NO: 995 - DNA, 21 bp, Artificial Sequence
  siRNA with 19 paired ribonucleotides and two unpaired nucleotides(“t”) on the 3′ end of each strand used in screens of DNA damaging agents camptothecin screen
gauggaaauauccugcagctt

SEQ ID NO: 996 - DNA, 21 bp, Artificial Sequence
  siRNA with 19 paired ribonucleotides and two unpaired nucleotides(“t”) on the 3′ end of each strand used in screens of DNA damaging agents camptothecin screen
ugcuguuagucgagaacugtt

SEQ ID NO: 997 - DNA, 21 bp, Artificial Sequence
  siRNA with 19 paired ribonucleotides and two unpaired nucleotides(“t”) on the 3′ end of each strand used in screens of DNA damaging agents camptothecin screen
acaagagguggaaucuccatt

SEQ ID NO: 998 - DNA, 21 bp, Artificial Sequence
  siRNA with 19 paired ribonucleotides and two unpaired nucleotides(“t”) on the 3′ end of each strand used in screens of DNA damaging agents camptothecin screen
gguuaucugcaggagucuutt

SEQ ID NO: 999 - DNA, 21 bp, Artificial Sequence
  siRNA with 19 paired ribonucleotides and two unpaired nucleotides(“t”) on the 3′ end of each strand used in screens of DNA damaging agents camptothecin screen
ucgaacagaugugcagugctt

SEQ ID NO: 1000 - DNA, 21 bp, Artificial Sequence
  siRNA with 19 paired ribonucleotides and two unpaired nucleotides(“t”) on the 3′ end of each strand used in screens of DNA damaging agents camptothecin screen
aaagcuaccgaugucagggtt

SEQ ID NO: 1001 - DNA, 21 bp, Artificial Sequence
  siRNA with 19 paired ribonucleotides and two unpaired nucleotides(“t”) on the 3′ end of each strand used in screens of DNA damaging agents camptothecin screen
aaaucccgaguuauccacctt

SEQ ID NO: 1002 - DNA, 21 bp, Artificial Sequence
  siRNA with 19 paired ribonucleotides and two unpaired nucleotides(“t”) on the 3′ end of each strand used in screens of DNA damaging agents camptothecin screen
ggcuaaugaaaggugcuggtt

SEQ ID NO: 1003 - DNA, 21 bp, Artificial Sequence
  siRNA with 19 paired ribonucleotides and two unpaired nucleotides(“t”) on the 3′ end of each strand used in screens of DNA damaging agents camptothecin screen
aagugacauuugggcucugtt

SEQ ID NO: 1004 - DNA, 21 bp, Artificial Sequence
  siRNA with 19 paired ribonucleotides and two unpaired nucleotides(“t”) on the 3′ end of each strand used in screens of DNA damaging agents camptothecin screen
augcacgugcugcuguacutt

SEQ ID NO: 1005 - DNA, 21 bp, Artificial Sequence
  siRNA with 19 paired ribonucleotides and two unpaired nucleotides(“t”) on the 3′ end of each strand used in screens of DNA damaging agents camptothecin screen
gaaggaccuucugauucugtt

SEQ ID NO: 1006 - DNA, 21 bp, Artificial Sequence
  siRNA with 19 paired ribonucleotides and two unpaired nucleotides(“t”) on the 3′ end of each strand used in screens of DNA damaging agents camptothecin screen
auccuucaacaaggcacagtt

SEQ ID NO: 1007 - DNA, 21 bp, Artificial Sequence
  siRNA with 19 paired ribonucleotides and two unpaired nucleotides(“t”) on the 3′ end of each strand used in screens of DNA damaging agents camptothecin screen
guaacuucagcagagcgaatt

SEQ ID NO: 1008 - DNA, 21 bp, Artificial Sequence
  siRNA with 19 paired ribonucleotides and two unpaired nucleotides(“t”) on the 3′ end of each strand used in screens of DNA damaging agents camptothecin screen
uacaugacuccauggcugutt

SEQ ID NO: 1009 - DNA, 21 bp, Artificial Sequence
  siRNA with 19 paired ribonucleotides and two unpaired nucleotides(“t”) on the 3′ end of each strand used in screens of DNA damaging agents camptothecin screen
aggauucucaugggaagggtt

SEQ ID NO: 1010 - DNA, 21 bp, Artificial Sequence
  siRNA with 19 paired ribonucleotides and two unpaired nucleotides(“t”) on the 3′ end of each strand used in screens of DNA damaging agents camptothecin screen
gaagaacaugauggggacutt

SEQ ID NO: 1011 - DNA, 21 bp, Artificial Sequence
  siRNA with 19 paired ribonucleotides and two unpaired nucleotides(“t”) on the 3′ end of each strand used in screens of DNA damaging agents camptothecin screen
gauugagcggccuguaacatt

SEQ ID NO: 1012 - DNA, 21 bp, Artificial Sequence
  siRNA with 19 paired ribonucleotides and two unpaired nucleotides(“t”) on the 3′ end of each strand used in screens of DNA damaging agents camptothecin screen
caacggcaacuacacgcugtt

SEQ ID NO: 1013 - DNA, 21 bp, Artificial Sequence
  siRNA with 19 paired ribonucleotides and two unpaired nucleotides(“t”) on the 3′ end of each strand used in screens of DNA damaging agents camptothecin screen
cgccacagcaucaaggaugtt

SEQ ID NO: 1014 - DNA, 21 bp, Artificial Sequence
  siRNA with 19 paired ribonucleotides and two unpaired nucleotides(“t”) on the 3′ end of each strand used in screens of DNA damaging agents camptothecin screen
gaguggucuccguuucgugtt

SEQ ID NO: 1015 - DNA, 21 bp, Artificial Sequence
  siRNA with 19 paired ribonucleotides and two unpaired nucleotides(“t”) on the 3′ end of each strand used in screens of DNA damaging agents camptothecin screen
gauacacaaagaugggcugtt

SEQ ID NO: 1016 - DNA, 21 bp, Artificial Sequence
  siRNA with 19 paired ribonucleotides and two unpaired nucleotides(“t”) on the 3′ end of each strand used in screens of DNA damaging agents camptothecin screen
aaacagcucaggcuuuguctt

SEQ ID NO: 1017 - DNA, 21 bp, Artificial Sequence
  siRNA with 19 paired ribonucleotides and two unpaired nucleotides(“t”) on the 3′ end of each strand used in screens of DNA damaging agents camptothecin screen
gaauaguggcuuaccgcuutt

SEQ ID NO: 1018 - DNA, 21 bp, Artificial Sequence
  siRNA with 19 paired ribonucleotides and two unpaired nucleotides(“t”) on the 3′ end of each strand used in screens of DNA damaging agents camptothecin screen
ccgcugucucgauauggautt

SEQ ID NO: 1019 - DNA, 21 bp, Artificial Sequence
  siRNA with 19 paired ribonucleotides and two unpaired nucleotides(“t”) on the 3′ end of each strand used in screens of DNA damaging agents camptothecin screen
ggaccgauuuuaccgaucctt

SEQ ID NO: 1020 - DNA, 21 bp, Artificial Sequence
  siRNA with 19 paired ribonucleotides and two unpaired nucleotides(“t”) on the 3′ end of each strand used in screens of DNA damaging agents camptothecin screen
uggauggcucugucaagcutt

SEQ ID NO: 1021 - DNA, 21 bp, Artificial Sequence
  siRNA with 19 paired ribonucleotides and two unpaired nucleotides(“t”) on the 3′ end of each strand used in screens of DNA damaging agents camptothecin screen
aauugcggauaugggacactt

SEQ ID NO: 1022 - DNA, 21 bp, Artificial Sequence
  siRNA with 19 paired ribonucleotides and two unpaired nucleotides(“t”) on the 3′ end of each strand used in screens of DNA damaging agents camptothecin screen
aguccaaagucugaucuggtt

SEQ ID NO: 1023 - DNA, 21 bp, Artificial Sequence
  siRNA with 19 paired ribonucleotides and two unpaired nucleotides(“t”) on the 3′ end of each strand used in screens of DNA damaging agents camptothecin screen
uuuccugugcaaaagacggtt

SEQ ID NO: 1024 - DNA, 21 bp, Artificial Sequence
  siRNA with 19 paired ribonucleotides and two unpaired nucleotides(“t”) on the 3′ end of each strand used in screens of DNA damaging agents camptothecin screen
aaagaaguaccagcaggugtt

SEQ ID NO: 1025 - DNA, 21 bp, Artificial Sequence
  siRNA with 19 paired ribonucleotides and two unpaired nucleotides(“t”) on the 3′ end of each strand used in screens of DNA damaging agents camptothecin screen
uccauccaccagagucuagtt

SEQ ID NO: 1026 - DNA, 21 bp, Artificial Sequence
  siRNA with 19 paired ribonucleotides and two unpaired nucleotides(“t”) on the 3′ end of each strand used in screens of DNA damaging agents camptothecin screen
uuugcugagcugcaucggatt

SEQ ID NO: 1027 - DNA, 21 bp, Artificial Sequence
  siRNA with 19 paired ribonucleotides and two unpaired nucleotides(“t”) on the 3′ end of each strand used in screens of DNA damaging agents camptothecin screen
aacccucauccacaggugatt

SEQ ID NO: 1028 - DNA, 21 bp, Artificial Sequence
  siRNA with 19 paired ribonucleotides and two unpaired nucleotides(“t”) on the 3′ end of each strand used in screens of DNA damaging agents camptothecin screen
ccgacugaguacaucgaugtt

SEQ ID NO: 1029 - DNA, 21 bp, Artificial Sequence
  siRNA with 19 paired ribonucleotides and two unpaired nucleotides(“t”) on the 3′ end of each strand used in screens of DNA damaging agents camptothecin screen
gccagauucgacugauugutt

SEQ ID NO: 1030 - DNA, 21 bp, Artificial Sequence
  siRNA with 19 paired ribonucleotides and two unpaired nucleotides(“t”) on the 3′ end of each strand used in screens of DNA damaging agents camptothecin screen
aacgaggaauccgacauuctt

SEQ ID NO: 1031 - DNA, 21 bp, Artificial Sequence
  siRNA with 19 paired ribonucleotides and two unpaired nucleotides(“t”) on the 3′ end of each strand used in screens of DNA damaging agents camptothecin screen
ugaugagcccauccuuucatt

SEQ ID NO: 1032 - DNA, 21 bp, Artificial Sequence
  siRNA with 19 paired ribonucleotides and two unpaired nucleotides(“t”) on the 3′ end of each strand used in screens of DNA damaging agents camptothecin screen
ugcuucaacggauguagcatt

SEQ ID NO: 1033 - DNA, 21 bp, Artificial Sequence
  siRNA with 19 paired ribonucleotides and two unpaired nucleotides(“t”) on the 3′ end of each strand used in screens of DNA damaging agents camptothecin screen
acagagacgccgaaaguactt

SEQ ID NO: 1034 - DNA, 21 bp, Artificial Sequence
  siRNA with 19 paired ribonucleotides and two unpaired nucleotides(“t”) on the 3′ end of each strand used in screens of DNA damaging agents camptothecin screen
cuagcgacauagaccuggutt

SEQ ID NO: 1035 - DNA, 21 bp, Artificial Sequence
  siRNA with 19 paired ribonucleotides and two unpaired nucleotides(“t”) on the 3′ end of each strand used in screens of DNA damaging agents camptothecin screen
gcaaaugaauuggccuggctt

SEQ ID NO: 1036 - DNA, 21 bp, Artificial Sequence
  siRNA with 19 paired ribonucleotides and two unpaired nucleotides(“t”) on the 3′ end of each strand used in screens of DNA damaging agents camptothecin screen
aaugacagaccucagacagtt

SEQ ID NO: 1037 - DNA, 21 bp, Artificial Sequence
  siRNA with 19 paired ribonucleotides and two unpaired nucleotides(“t”) on the 3′ end of each strand used in screens of DNA damaging agents camptothecin screen
uaagccucaugaagagccutt

SEQ ID NO: 1038 - DNA, 21 bp, Artificial Sequence
  siRNA with 19 paired ribonucleotides and two unpaired nucleotides(“t”) on the 3′ end of each strand used in screens of DNA damaging agents camptothecin screen
ugucaguacugucgguuuctt

SEQ ID NO: 1039 - DNA, 21 bp, Artificial Sequence
  siRNA with 19 paired ribonucleotides and two unpaired nucleotides(“t”) on the 3′ end of each strand used in screens of DNA damaging agents camptothecin screen
aagcauauccgucugucagtt

SEQ ID NO: 1040 - DNA, 21 bp, Artificial Sequence
  siRNA with 19 paired ribonucleotides and two unpaired nucleotides(“t”) on the 3′ end of each strand used in screens of DNA damaging agents camptothecin screen
aggcuuccaaaucugaugctt

SEQ ID NO: 1041 - DNA, 21 bp, Artificial Sequence
  siRNA with 19 paired ribonucleotides and two unpaired nucleotides(“t”) on the 3′ end of each strand used in screens of DNA damaging agents camptothecin screen
ggaacucuuugaagguguctt

SEQ ID NO: 1042 - DNA, 21 bp, Artificial Sequence
  siRNA with 19 paired ribonucleotides and two unpaired nucleotides(“t”) on the 3′ end of each strand used in screens of DNA damaging agents camptothecin screen
gaauggggucaacgauauctt

SEQ ID NO: 1043 - DNA, 21 bp, Artificial Sequence
  siRNA with 19 paired ribonucleotides and two unpaired nucleotides(“t”) on the 3′ end of each strand used in screens of DNA damaging agents camptothecin screen
ggacgagacuuccucuugatt

SEQ ID NO: 1044 - DNA, 21 bp, Artificial Sequence
  siRNA with 19 paired ribonucleotides and two unpaired nucleotides(“t”) on the 3′ end of each strand used in screens of DNA damaging agents camptothecin screen
guguggcaaggaguuuucutt

SEQ ID NO: 1045 - DNA, 21 bp, Artificial Sequence
  siRNA with 19 paired ribonucleotides and two unpaired nucleotides(“t”) on the 3′ end of each strand used in screens of DNA damaging agents camptothecin screen
agagcaugcauuuuuccggtt

SEQ ID NO: 1046 - DNA, 21 bp, Artificial Sequence
  siRNA with 19 paired ribonucleotides and two unpaired nucleotides(“t”) on the 3′ end of each strand used in screens of DNA damaging agents camptothecin screen
ggagccccaugcuguauuutt

SEQ ID NO: 1047 - DNA, 21 bp, Artificial Sequence
  siRNA with 19 paired ribonucleotides and two unpaired nucleotides(“t”) on the 3′ end of each strand used in screens of DNA damaging agents camptothecin screen
uuggauguuagcgguacuctt

SEQ ID NO: 1048 - DNA, 21 bp, Artificial Sequence
  siRNA with 19 paired ribonucleotides and two unpaired nucleotides(“t”) on the 3′ end of each strand used in screens of DNA damaging agents camptothecin screen
cacaagaucgucuacagggtt

SEQ ID NO: 1049 - DNA, 21 bp, Artificial Sequence
  siRNA with 19 paired ribonucleotides and two unpaired nucleotides(“t”) on the 3′ end of each strand used in screens of DNA damaging agents camptothecin screen
caccagugaagucagcacutt

SEQ ID NO: 1050 - DNA, 21 bp, Artificial Sequence
  siRNA with 19 paired ribonucleotides and two unpaired nucleotides(“t”) on the 3′ end of each strand used in screens of DNA damaging agents camptothecin screen
gauuucaaguuccuggcggtt

SEQ ID NO: 1051 - DNA, 21 bp, Artificial Sequence
  siRNA with 19 paired ribonucleotides and two unpaired nucleotides(“t”) on the 3′ end of each strand used in screens of DNA damaging agents camptothecin screen
aaugaaugaggaggguaggtt

SEQ ID NO: 1052 - DNA, 21 bp, Artificial Sequence
  siRNA with 19 paired ribonucleotides and two unpaired nucleotides(“t”) on the 3′ end of each strand used in screens of DNA damaging agents camptothecin screen
ccuucaucacccugguguutt

SEQ ID NO: 1053 - DNA, 21 bp, Artificial Sequence
  siRNA with 19 paired ribonucleotides and two unpaired nucleotides(“t”) on the 3′ end of each strand used in screens of DNA damaging agents camptothecin screen
guucccugaaugugguuuctt

SEQ ID NO: 1054 - DNA, 21 bp, Artificial Sequence
  siRNA with 19 paired ribonucleotides and two unpaired nucleotides(“t”) on the 3′ end of each strand used in screens of DNA damaging agents camptothecin screen
accaagcagccagcuauactt

SEQ ID NO: 1055 - DNA, 21 bp, Artificial Sequence
  siRNA with 19 paired ribonucleotides and two unpaired nucleotides(“t”) on the 3′ end of each strand used in screens of DNA damaging agents camptothecin screen
cuaaugacaucugggaccutt

SEQ ID NO: 1056 - DNA, 21 bp, Artificial Sequence
  siRNA with 19 paired ribonucleotides and two unpaired nucleotides(“t”) on the 3′ end of each strand used in screens of DNA damaging agents camptothecin screen
cuacgaaggaaaacgucagtt

SEQ ID NO: 1057 - DNA, 21 bp, Artificial Sequence
  siRNA with 19 paired ribonucleotides and two unpaired nucleotides(“t”) on the 3′ end of each strand used in screens of DNA damaging agents camptothecin screen
agacagugaagcugguugutt

SEQ ID NO: 1058 - DNA, 21 bp, Artificial Sequence
  siRNA with 19 paired ribonucleotides and two unpaired nucleotides(“t”) on the 3′ end of each strand used in screens of DNA damaging agents camptothecin screen
gaauuucugaggacgagcutt

SEQ ID NO: 1059 - DNA, 21 bp, Artificial Sequence
  siRNA with 19 paired ribonucleotides and two unpaired nucleotides(“t”) on the 3′ end of each strand used in screens of DNA damaging agents camptothecin screen
ucagauuugggccagaguutt

SEQ ID NO: 1060 - DNA, 21 bp, Artificial Sequence
  siRNA with 19 paired ribonucleotides and two unpaired nucleotides(“t”) on the 3′ end of each strand used in screens of DNA damaging agents camptothecin screen
uggaggggaaugcucagaatt

SEQ ID NO: 1061 - DNA, 21 bp, Artificial Sequence
  siRNA with 19 paired ribonucleotides and two unpaired nucleotides(“t”) on the 3′ end of each strand used in screens of DNA damaging agents camptothecin screen
aaggcagcuaaaggaagugtt

SEQ ID NO: 1062 - DNA, 21 bp, Artificial Sequence
  siRNA with 19 paired ribonucleotides and two unpaired nucleotides(“t”) on the 3′ end of each strand used in screens of DNA damaging agents camptothecin screen
uaaagauggcacuuucccgtt

SEQ ID NO: 1063 - DNA, 21 bp, Artificial Sequence
  siRNA with 19 paired ribonucleotides and two unpaired nucleotides(“t”) on the 3′ end of each strand used in screens of DNA damaging agents camptothecin screen
aacacccucugcaaaguugtt

SEQ ID NO: 1064 - DNA, 21 bp, Artificial Sequence
  siRNA with 19 paired ribonucleotides and two unpaired nucleotides(“t”) on the 3′ end of each strand used in screens of DNA damaging agents camptothecin screen
ccgugguucuuuggcugcatt

SEQ ID NO: 1065 - DNA, 21 bp, Artificial Sequence
  siRNA with 19 paired ribonucleotides and two unpaired nucleotides(“t”) on the 3′ end of each strand used in screens of DNA damaging agents camptothecin screen
ucaggcuuauccgaugugctt

SEQ ID NO: 1066 - DNA, 21 bp, Artificial Sequence
  siRNA with 19 paired ribonucleotides and two unpaired nucleotides(“t”) on the 3′ end of each strand used in screens of DNA damaging agents camptothecin screen
aacagccaagcuuuucugctt

SEQ ID NO: 1067 - DNA, 21 bp, Artificial Sequence
  siRNA with 19 paired ribonucleotides and two unpaired nucleotides(“t”) on the 3′ end of each strand used in screens of DNA damaging agents camptothecin screen
ggcuuugggaacugucaactt

SEQ ID NO: 1068 - DNA, 21 bp, Artificial Sequence
  siRNA with 19 paired ribonucleotides and two unpaired nucleotides(“t”) on the 3′ end of each strand used in screens of DNA damaging agents camptothecin screen
ucuguugcagucuccuucatt

SEQ ID NO: 1069 - DNA, 21 bp, Artificial Sequence
  siRNA with 19 paired ribonucleotides and two unpaired nucleotides(“t”) on the 3′ end of each strand used in screens of DNA damaging agents camptothecin screen
agacagguuucuguagaagtt

SEQ ID NO: 1070 - DNA, 21 bp, Artificial Sequence
  siRNA with 19 paired ribonucleotides and two unpaired nucleotides(“t”) on the 3′ end of each strand used in screens of DNA damaging agents camptothecin screen
guccagaucgauaucuuagtt

SEQ ID NO: 1071 - DNA, 21 bp, Artificial Sequence
  siRNA with 19 paired ribonucleotides and two unpaired nucleotides(“t”) on the 3′ end of each strand used in screens of DNA damaging agents camptothecin screen
guuuagccaagagaaucagtt

SEQ ID NO: 1072 - DNA, 21 bp, Artificial Sequence
  siRNA with 19 paired ribonucleotides and two unpaired nucleotides(“t”) on the 3′ end of each strand used in screens of DNA damaging agents camptothecin screen
aaugaagagguccaagggatt

SEQ ID NO: 1073 - DNA, 21 bp, Artificial Sequence
  siRNA with 19 paired ribonucleotides and two unpaired nucleotides(“t”) on the 3′ end of each strand used in screens of DNA damaging agents camptothecin screen
aggacccgugcaucuuacutt

SEQ ID NO: 1074 - DNA, 21 bp, Artificial Sequence
  siRNA with 19 paired ribonucleotides and two unpaired nucleotides(“t”) on the 3′ end of each strand used in screens of DNA damaging agents camptothecin screen
gcuaaaagacaggagugugtt

SEQ ID NO: 1075 - DNA, 21 bp, Artificial Sequence
  siRNA with 19 paired ribonucleotides and two unpaired nucleotides(“t”) on the 3′ end of each strand used in screens of DNA damaging agents camptothecin screen
caguguaugcucgaaaucctt

SEQ ID NO: 1076 - DNA, 21 bp, Artificial Sequence
  siRNA with 19 paired ribonucleotides and two unpaired nucleotides(“t”) on the 3′ end of each strand used in screens of DNA damaging agents camptothecin screen
gaugagcgaguaguuggcatt

SEQ ID NO: 1077 - DNA, 21 bp, Artificial Sequence
  siRNA with 19 paired ribonucleotides and two unpaired nucleotides(“t”) on the 3′ end of each strand used in screens of DNA damaging agents camptothecin screen
uuggaaagugccagcauuctt

SEQ ID NO: 1078 - DNA, 21 bp, Artificial Sequence
  siRNA with 19 paired ribonucleotides and two unpaired nucleotides(“t”) on the 3′ end of each strand used in screens of DNA damaging agents camptothecin screen
aacugaugaccuggggucutt

SEQ ID NO: 1079 - DNA, 21 bp, Artificial Sequence
  siRNA with 19 paired ribonucleotides and two unpaired nucleotides(“t”) on the 3′ end of each strand used in screens of DNA damaging agents camptothecin screen
caagcagagagcucaccuatt

SEQ ID NO: 1080 - DNA, 21 bp, Artificial Sequence
  siRNA with 19 paired ribonucleotides and two unpaired nucleotides(“t”) on the 3′ end of each strand used in screens of DNA damaging agents camptothecin screen
gaaaacaagcaauccgcagtt

SEQ ID NO: 1081 - DNA, 21 bp, Artificial Sequence
  siRNA with 19 paired ribonucleotides and two unpaired nucleotides(“t”) on the 3′ end of each strand used in screens of DNA damaging agents camptothecin screen
cuucgcagaaaagagucggtt

SEQ ID NO: 1082 - DNA, 21 bp, Artificial Sequence
  siRNA with 19 paired ribonucleotides and two unpaired nucleotides(“t”) on the 3′ end of each strand used in screens of DNA damaging agents camptothecin screen
gacauccaggauccacugutt

SEQ ID NO: 1083 - DNA, 21 bp, Artificial Sequence
  siRNA with 19 paired ribonucleotides and two unpaired nucleotides(“t”) on the 3′ end of each strand used in screens of DNA damaging agents camptothecin screen
gugugccgaacuucacuactt

SEQ ID NO: 1084 - DNA, 21 bp, Artificial Sequence
  siRNA with 19 paired ribonucleotides and two unpaired nucleotides(“t”) on the 3′ end of each strand used in screens of DNA damaging agents camptothecin screen
aguacacagcagguucauctt

SEQ ID NO: 1085 - DNA, 21 bp, Artificial Sequence
  siRNA with 19 paired ribonucleotides and two unpaired nucleotides(“t”) on the 3′ end of each strand used in screens of DNA damaging agents camptothecin screen
cgugaaugaggagaacccutt

SEQ ID NO: 1086 - DNA, 21 bp, Artificial Sequence
  siRNA with 19 paired ribonucleotides and two unpaired nucleotides(“t”) on the 3′ end of each strand used in screens of DNA damaging agents camptothecin screen
guuuaaggaggggccugugtt

SEQ ID NO: 1087 - DNA, 21 bp, Artificial Sequence
  siRNA with 19 paired ribonucleotides and two unpaired nucleotides(“t”) on the 3′ end of each strand used in screens of DNA damaging agents camptothecin screen
ccuccuuucaaaccugcuutt

SEQ ID NO: 1088 - DNA, 21 bp, Artificial Sequence
  siRNA with 19 paired ribonucleotides and two unpaired nucleotides(“t”) on the 3′ end of each strand used in screens of DNA damaging agents camptothecin screen
gagguucuguuuacagaggtt

SEQ ID NO: 1089 - DNA, 21 bp, Artificial Sequence
  siRNA with 19 paired ribonucleotides and two unpaired nucleotides(“t”) on the 3′ end of each strand used in screens of DNA damaging agents camptothecin screen
ucagccagugcagauucaatt

SEQ ID NO: 1090 - DNA, 21 bp, Artificial Sequence
  siRNA with 19 paired ribonucleotides and two unpaired nucleotides(“t”) on the 3′ end of each strand used in screens of DNA damaging agents camptothecin screen
agagccuuaugaucgagcatt

SEQ ID NO: 1091 - DNA, 21 bp, Artificial Sequence
  siRNA with 19 paired ribonucleotides and two unpaired nucleotides(“t”) on the 3′ end of each strand used in screens of DNA damaging agents camptothecin screen
cucuaucaugccugcuccutt

SEQ ID NO: 1092 - DNA, 21 bp, Artificial Sequence
  siRNA with 19 paired ribonucleotides and two unpaired nucleotides(“t”) on the 3′ end of each strand used in screens of DNA damaging agents camptothecin screen
gagaaggaccugugaaacutt

SEQ ID NO: 1093 - DNA, 21 bp, Artificial Sequence
  siRNA with 19 paired ribonucleotides and two unpaired nucleotides(“t”) on the 3′ end of each strand used in screens of DNA damaging agents camptothecin screen
aagaaccaggagauaugggtt

SEQ ID NO: 1094 - DNA, 21 bp, Artificial Sequence
  siRNA with 19 paired ribonucleotides and two unpaired nucleotides(“t”) on the 3′ end of each strand used in screens of DNA damaging agents camptothecin screen
ggucucugguguuuguaagtt

SEQ ID NO: 1095 - DNA, 21 bp, Artificial Sequence
  siRNA with 19 paired ribonucleotides and two unpaired nucleotides(“t”) on the 3′ end of each strand used in screens of DNA damaging agents camptothecin screen
uuugcccugcagacuuugctt

SEQ ID NO: 1096 - DNA, 21 bp, Artificial Sequence
  siRNA with 19 paired ribonucleotides and two unpaired nucleotides(“t”) on the 3′ end of each strand used in screens of DNA damaging agents camptothecin screen
gaaccggaugcaguugagctt

SEQ ID NO: 1097 - DNA, 21 bp, Artificial Sequence
  siRNA with 19 paired ribonucleotides and two unpaired nucleotides(“t”) on the 3′ end of each strand used in screens of DNA damaging agents camptothecin screen
gccggaauacaagaacgggtt

SEQ ID NO: 1098 - DNA, 21 bp, Artificial Sequence
  siRNA with 19 paired ribonucleotides and two unpaired nucleotides(“t”) on the 3′ end of each strand used in screens of DNA damaging agents camptothecin screen
guggcucuuauccgcaugatt

SEQ ID NO: 1099 - DNA, 21 bp, Artificial Sequence
  siRNA with 19 paired ribonucleotides and two unpaired nucleotides(“t”) on the 3′ end of each strand used in screens of DNA damaging agents camptothecin screen
cgcuuauggaacgugauactt

SEQ ID NO: 1100 - DNA, 21 bp, Artificial Sequence
  siRNA with 19 paired ribonucleotides and two unpaired nucleotides(“t”) on the 3′ end of each strand used in screens of DNA damaging agents camptothecin screen
gcaacagaauggcagcgautt

SEQ ID NO: 1101 - DNA, 21 bp, Artificial Sequence
  siRNA with 19 paired ribonucleotides and two unpaired nucleotides(“t”) on the 3′ end of each strand used in screens of DNA damaging agents camptothecin screen
guucuaaucggaucuggcutt

SEQ ID NO: 1102 - DNA, 21 bp, Artificial Sequence
  siRNA with 19 paired ribonucleotides and two unpaired nucleotides(“t”) on the 3′ end of each strand used in screens of DNA damaging agents camptothecin screen
augccacaggccaccuauatt

SEQ ID NO: 1103 - DNA, 21 bp, Artificial Sequence
  siRNA with 19 paired ribonucleotides and two unpaired nucleotides(“t”) on the 3′ end of each strand used in screens of DNA damaging agents camptothecin screen
cgaccugcagcaauaccautt

SEQ ID NO: 1104 - DNA, 21 bp, Artificial Sequence
  siRNA with 19 paired ribonucleotides and two unpaired nucleotides(“t”) on the 3′ end of each strand used in screens of DNA damaging agents camptothecin screen
gaaucacaugccacuuuggtt

SEQ ID NO: 1105 - DNA, 21 bp, Artificial Sequence
  siRNA with 19 paired ribonucleotides and two unpaired nucleotides(“t”) on the 3′ end of each strand used in screens of DNA damaging agents camptothecin screen
acacagaucugccucuaugtt

SEQ ID NO: 1106 - DNA, 21 bp, Artificial Sequence
  siRNA with 19 paired ribonucleotides and two unpaired nucleotides(“t”) on the 3′ end of each strand used in screens of DNA damaging agents camptothecin screen
cccuacaauaaaggccggutt

SEQ ID NO: 1107 - DNA, 21 bp, Artificial Sequence
  siRNA with 19 paired ribonucleotides and two unpaired nucleotides(“t”) on the 3′ end of each strand used in screens of DNA damaging agents camptothecin screen
uuaggaagguccuucagggtt

SEQ ID NO: 1108 - DNA, 21 bp, Artificial Sequence
  siRNA with 19 paired ribonucleotides and two unpaired nucleotides(“t”) on the 3′ end of each strand used in screens of DNA damaging agents camptothecin screen
ccuguggaaccugaaaccatt

SEQ ID NO: 1109 - DNA, 21 bp, Artificial Sequence
  siRNA with 19 paired ribonucleotides and two unpaired nucleotides(“t”) on the 3′ end of each strand used in screens of DNA damaging agents camptothecin screen
gucuaugaugcuguugccctt

SEQ ID NO: 1110 - DNA, 21 bp, Artificial Sequence
  siRNA with 19 paired ribonucleotides and two unpaired nucleotides(“t”) on the 3′ end of each strand used in screens of DNA damaging agents camptothecin screen
ugagaugauucagaaggggtt

SEQ ID NO: 1111 - DNA, 21 bp, Artificial Sequence
  siRNA with 19 paired ribonucleotides and two unpaired nucleotides(“t”) on the 3′ end of each strand used in screens of DNA damaging agents camptothecin screen
caccauuuuggauggcucctt

SEQ ID NO: 1112 - DNA, 21 bp, Artificial Sequence
  siRNA with 19 paired ribonucleotides and two unpaired nucleotides(“t”) on the 3′ end of each strand used in screens of DNA damaging agents camptothecin screen
ggaaaaccagaucaacagctt

SEQ ID NO: 1113 - DNA, 21 bp, Artificial Sequence
  siRNA with 19 paired ribonucleotides and two unpaired nucleotides(“t”) on the 3′ end of each strand used in screens of DNA damaging agents camptothecin screen
uucuggauggcuugccucatt

SEQ ID NO: 1114 - DNA, 21 bp, Artificial Sequence
  siRNA with 19 paired ribonucleotides and two unpaired nucleotides(“t”) on the 3′ end of each strand used in screens of DNA damaging agents camptothecin screen
acagucuuaggaaucgugctt

SEQ ID NO: 1115 - DNA, 21 bp, Artificial Sequence
  siRNA with 19 paired ribonucleotides and two unpaired nucleotides(“t”) on the 3′ end of each strand used in screens of DNA damaging agents camptothecin screen
gcacaaaagcuugucuccatt

SEQ ID NO: 1116 - DNA, 21 bp, Artificial Sequence
  siRNA with 19 paired ribonucleotides and two unpaired nucleotides(“t”) on the 3′ end of each strand used in screens of DNA damaging agents camptothecin screen
uugcagauuuugggugguctt

SEQ ID NO: 1117 - DNA, 21 bp, Artificial Sequence
  siRNA with 19 paired ribonucleotides and two unpaired nucleotides(“t”) on the 3′ end of each strand used in screens of DNA damaging agents camptothecin screen
aaagaccccguguaaaccutt

SEQ ID NO: 1118 - DNA, 21 bp, Artificial Sequence
  siRNA with 19 paired ribonucleotides and two unpaired nucleotides(“t”) on the 3′ end of each strand used in screens of DNA damaging agents camptothecin screen
accucaaggccagcucaautt

SEQ ID NO: 1119 - DNA, 21 bp, Artificial Sequence
  siRNA with 19 paired ribonucleotides and two unpaired nucleotides(“t”) on the 3′ end of each strand used in screens of DNA damaging agents camptothecin screen
auggggcauuuuguugggatt

SEQ ID NO: 1120 - DNA, 21 bp, Artificial Sequence
  siRNA with 19 paired ribonucleotides and two unpaired nucleotides(“t”) on the 3′ end of each strand used in screens of DNA damaging agents camptothecin screen
caccaagaaguucaucucctt

SEQ ID NO: 1121 - DNA, 21 bp, Artificial Sequence
  siRNA with 19 paired ribonucleotides and two unpaired nucleotides(“t”) on the 3′ end of each strand used in screens of DNA damaging agents camptothecin screen
gagugucuggagcaaguugtt

SEQ ID NO: 1122 - DNA, 21 bp, Artificial Sequence
  siRNA with 19 paired ribonucleotides and two unpaired nucleotides(“t”) on the 3′ end of each strand used in screens of DNA damaging agents camptothecin screen
guuuggaagaaccccacautt

SEQ ID NO: 1123 - DNA, 21 bp, Artificial Sequence
  siRNA with 19 paired ribonucleotides and two unpaired nucleotides(“t”) on the 3′ end of each strand used in screens of DNA damaging agents camptothecin screen
gacaugauucagccacagatt

SEQ ID NO: 1124 - DNA, 21 bp, Artificial Sequence
  siRNA with 19 paired ribonucleotides and two unpaired nucleotides(“t”) on the 3′ end of each strand used in screens of DNA damaging agents camptothecin screen
uuccucgagauaggccguutt

SEQ ID NO: 1125 - DNA, 21 bp, Artificial Sequence
  siRNA with 19 paired ribonucleotides and two unpaired nucleotides(“t”) on the 3′ end of each strand used in screens of DNA damaging agents camptothecin screen
uuugggaggucaguuguuctt

SEQ ID NO: 1126 - DNA, 21 bp, Artificial Sequence
  siRNA with 19 paired ribonucleotides and two unpaired nucleotides(“t”) on the 3′ end of each strand used in screens of DNA damaging agents camptothecin screen
gaugccagaaguggacuautt

SEQ ID NO: 1127 - DNA, 21 bp, Artificial Sequence
  siRNA with 19 paired ribonucleotides and two unpaired nucleotides(“t”) on the 3′ end of each strand used in screens of DNA damaging agents camptothecin screen
uaccuggaagcaauucacctt

SEQ ID NO: 1128 - DNA, 21 bp, Artificial Sequence
  siRNA with 19 paired ribonucleotides and two unpaired nucleotides(“t”) on the 3′ end of each strand used in screens of DNA damaging agents camptothecin screen
uuaugcugcauuuggcuggtt

SEQ ID NO: 1129 - DNA, 21 bp, Artificial Sequence
  siRNA with 19 paired ribonucleotides and two unpaired nucleotides(“t”) on the 3′ end of each strand used in screens of DNA damaging agents camptothecin screen
acauucccuggaguguacatt

SEQ ID NO: 1130 - DNA, 21 bp, Artificial Sequence
  siRNA with 19 paired ribonucleotides and two unpaired nucleotides(“t”) on the 3′ end of each strand used in screens of DNA damaging agents camptothecin screen
gaggauuuagcggcauuugtt

SEQ ID NO: 1131 - DNA, 21 bp, Artificial Sequence
  siRNA with 19 paired ribonucleotides and two unpaired nucleotides(“t”) on the 3′ end of each strand used in screens of DNA damaging agents camptothecin screen
gcugcuggacugcauaaagtt

SEQ ID NO: 1132 - DNA, 21 bp, Artificial Sequence
  siRNA with 19 paired ribonucleotides and two unpaired nucleotides(“t”) on the 3′ end of each strand used in screens of DNA damaging agents camptothecin screen
gauccucccugucuaugagtt

SEQ ID NO: 1133 - DNA, 21 bp, Artificial Sequence
  siRNA with 19 paired ribonucleotides and two unpaired nucleotides(“t”) on the 3′ end of each strand used in screens of DNA damaging agents camptothecin screen
gcaaagaaauuggacgaggtt

SEQ ID NO: 1134 - DNA, 21 bp, Artificial Sequence
  siRNA with 19 paired ribonucleotides and two unpaired nucleotides(“t”) on the 3′ end of each strand used in screens of DNA damaging agents camptothecin screen
ggcgaaaacaucggguuugtt

SEQ ID NO: 1135 - DNA, 21 bp, Artificial Sequence
  siRNA with 19 paired ribonucleotides and two unpaired nucleotides(“t”) on the 3′ end of each strand used in screens of DNA damaging agents camptothecin screen
caaaugggacaaaguggactt

SEQ ID NO: 1136 - DNA, 21 bp, Artificial Sequence
  siRNA with 19 paired ribonucleotides and two unpaired nucleotides(“t”) on the 3′ end of each strand used in screens of DNA damaging agents camptothecin screen
cuuugaccugaagggcucutt

SEQ ID NO: 1137 - DNA, 21 bp, Artificial Sequence
  siRNA with 19 paired ribonucleotides and two unpaired nucleotides(“t”) on the 3′ end of each strand used in screens of DNA damaging agents camptothecin screen
uccaguccuucaaaccagatt

SEQ ID NO: 1138 - DNA, 21 bp, Artificial Sequence
  siRNA with 19 paired ribonucleotides and two unpaired nucleotides(“t”) on the 3′ end of each strand used in screens of DNA damaging agents camptothecin screen
aaacaggagguguugaagctt

SEQ ID NO: 1139 - DNA, 21 bp, Artificial Sequence
  siRNA with 19 paired ribonucleotides and two unpaired nucleotides(“t”) on the 3′ end of each strand used in screens of DNA damaging agents camptothecin screen
aaguggagcagaacagucgtt

SEQ ID NO: 1140 - DNA, 21 bp, Artificial Sequence
  siRNA with 19 paired ribonucleotides and two unpaired nucleotides(“t”) on the 3′ end of each strand used in screens of DNA damaging agents camptothecin screen
ggacaauccacagagaucutt

SEQ ID NO: 1141 - DNA, 21 bp, Artificial Sequence
  siRNA with 19 paired ribonucleotides and two unpaired nucleotides(“t”) on the 3′ end of each strand used in screens of DNA damaging agents camptothecin screen
aucggcucuggagaauuugtt

SEQ ID NO: 1142 - DNA, 21 bp, Artificial Sequence
  siRNA with 19 paired ribonucleotides and two unpaired nucleotides(“t”) on the 3′ end of each strand used in screens of DNA damaging agents camptothecin screen
caaggaucuccaguccacatt

SEQ ID NO: 1143 - DNA, 21 bp, Artificial Sequence
  siRNA with 19 paired ribonucleotides and two unpaired nucleotides(“t”) on the 3′ end of each strand used in screens of DNA damaging agents camptothecin screen
uguaccuguguguccaucutt

SEQ ID NO: 1144 - DNA, 21 bp, Artificial Sequence
  siRNA with 19 paired ribonucleotides and two unpaired nucleotides(“t”) on the 3′ end of each strand used in screens of DNA damaging agents camptothecin screen
aaaugccugucucuagcugtt

SEQ ID NO: 1145 - DNA, 21 bp, Artificial Sequence
  siRNA with 19 paired ribonucleotides and two unpaired nucleotides(“t”) on the 3′ end of each strand used in screens of DNA damaging agents camptothecin screen
auggccaguuuucugguagtt

SEQ ID NO: 1146 - DNA, 21 bp, Artificial Sequence
  siRNA with 19 paired ribonucleotides and two unpaired nucleotides(“t”) on the 3′ end of each strand used in screens of DNA damaging agents camptothecin screen
ccugggcauuguugagguutt

SEQ ID NO: 1147 - DNA, 21 bp, Artificial Sequence
  siRNA with 19 paired ribonucleotides and two unpaired nucleotides(“t”) on the 3′ end of each strand used in screens of DNA damaging agents camptothecin screen
acaguuuugucacuggccctt

SEQ ID NO: 1148 - DNA, 21 bp, Artificial Sequence
  siRNA with 19 paired ribonucleotides and two unpaired nucleotides(“t”) on the 3′ end of each strand used in screens of DNA damaging agents camptothecin screen
caaaauggacucccugcuctt

SEQ ID NO: 1149 - DNA, 21 bp, Artificial Sequence
  siRNA with 19 paired ribonucleotides and two unpaired nucleotides(“t”) on the 3′ end of each strand used in screens of DNA damaging agents camptothecin screen
ccaggggaaaucugcaaugtt

SEQ ID NO: 1150 - DNA, 21 bp, Artificial Sequence
  siRNA with 19 paired ribonucleotides and two unpaired nucleotides(“t”) on the 3′ end of each strand used in screens of DNA damaging agents camptothecin screen
acggcguuuaucuucgcuatt

SEQ ID NO: 1151 - DNA, 21 bp, Artificial Sequence
  siRNA with 19 paired ribonucleotides and two unpaired nucleotides(“t”) on the 3′ end of each strand used in screens of DNA damaging agents camptothecin screen
cccucuugccauccugaugtt

SEQ ID NO: 1152 - DNA, 21 bp, Artificial Sequence
  siRNA with 19 paired ribonucleotides and two unpaired nucleotides(“t”) on the 3′ end of each strand used in screens of DNA damaging agents camptothecin screen
cuauuuauugugcuggguctt

SEQ ID NO: 1153 - DNA, 21 bp, Artificial Sequence
  siRNA with 19 paired ribonucleotides and two unpaired nucleotides(“t”) on the 3′ end of each strand used in screens of DNA damaging agents camptothecin screen
auuugcccgcgcauuugugtt

SEQ ID NO: 1154 - DNA, 21 bp, Artificial Sequence
  siRNA with 19 paired ribonucleotides and two unpaired nucleotides(“t”) on the 3′ end of each strand used in screens of DNA damaging agents camptothecin screen
aacgggcgauuaucucuggtt

SEQ ID NO: 1155 - DNA, 21 bp, Artificial Sequence
  siRNA with 19 paired ribonucleotides and two unpaired nucleotides(“t”) on the 3′ end of each strand used in screens of DNA damaging agents camptothecin screen
agaagaugaauggucuggctt

SEQ ID NO: 1156 - DNA, 21 bp, Artificial Sequence
  siRNA with 19 paired ribonucleotides and two unpaired nucleotides(“t”) on the 3′ end of each strand used in screens of DNA damaging agents camptothecin screen
cacuggcacacugcucuuatt

SEQ ID NO: 1157 - DNA, 21 bp, Artificial Sequence
  siRNA with 19 paired ribonucleotides and two unpaired nucleotides(“t”) on the 3′ end of each strand used in screens of DNA damaging agents camptothecin screen
gacaagauaccggugcuuctt

SEQ ID NO: 1158 - DNA, 21 bp, Artificial Sequence
  siRNA with 19 paired ribonucleotides and two unpaired nucleotides(“t”) on the 3′ end of each strand used in screens of DNA damaging agents camptothecin screen
gacaccaaaggagacauactt

SEQ ID NO: 1159 - DNA, 21 bp, Artificial Sequence
  siRNA with 19 paired ribonucleotides and two unpaired nucleotides(“t”) on the 3′ end of each strand used in screens of DNA damaging agents camptothecin screen
agaccuaaucacacggaugtt

SEQ ID NO: 1160 - DNA, 21 bp, Artificial Sequence
  siRNA with 19 paired ribonucleotides and two unpaired nucleotides(“t”) on the 3′ end of each strand used in screens of DNA damaging agents camptothecin screen
agauagcggguucaccuactt

SEQ ID NO: 1161 - DNA, 21 bp, Artificial Sequence
  siRNA with 19 paired ribonucleotides and two unpaired nucleotides(“t”) on the 3′ end of each strand used in screens of DNA damaging agents camptothecin screen
guugacagacuuuggguuctt

SEQ ID NO: 1162 - DNA, 21 bp, Artificial Sequence
  siRNA with 19 paired ribonucleotides and two unpaired nucleotides(“t”) on the 3′ end of each strand used in screens of DNA damaging agents camptothecin screen
acuccaucugguugaccugtt

SEQ ID NO: 1163 - DNA, 21 bp, Artificial Sequence
  siRNA with 19 paired ribonucleotides and two unpaired nucleotides(“t”) on the 3′ end of each strand used in screens of DNA damaging agents camptothecin screen
gauucagguggaacugaactt

SEQ ID NO: 1164 - DNA, 21 bp, Artificial Sequence
  siRNA with 19 paired ribonucleotides and two unpaired nucleotides(“t”) on the 3′ end of each strand used in screens of DNA damaging agents camptothecin screen
gcaccaagcuccucugaugtt

SEQ ID NO: 1165 - DNA, 21 bp, Artificial Sequence
  siRNA with 19 paired ribonucleotides and two unpaired nucleotides(“t”) on the 3′ end of each strand used in screens of DNA damaging agents camptothecin screen
accgacacuuuggcuuccatt

SEQ ID NO: 1166 - DNA, 21 bp, Artificial Sequence
  siRNA with 19 paired ribonucleotides and two unpaired nucleotides(“t”) on the 3′ end of each strand used in screens of DNA damaging agents camptothecin screen
gaugagcgcgggaauguugtt

SEQ ID NO: 1167 - DNA, 21 bp, Artificial Sequence
  siRNA with 19 paired ribonucleotides and two unpaired nucleotides(“t”) on the 3′ end of each strand used in screens of DNA damaging agents camptothecin screen
uggccgaggccuucaagcutt

SEQ ID NO: 1168 - DNA, 21 bp, Artificial Sequence
  siRNA with 19 paired ribonucleotides and two unpaired nucleotides(“t”) on the 3′ end of each strand used in screens of DNA damaging agents camptothecin screen
caucaaucacucucugcugtt

SEQ ID NO: 1169 - DNA, 21 bp, Artificial Sequence
  siRNA with 19 paired ribonucleotides and two unpaired nucleotides(“t”) on the 3′ end of each strand used in screens of DNA damaging agents camptothecin screen
cuaacccaggauguucaggtt

SEQ ID NO: 1170 - DNA, 21 bp, Artificial Sequence
  siRNA with 19 paired ribonucleotides and two unpaired nucleotides(“t”) on the 3′ end of each strand used in screens of DNA damaging agents camptothecin screen
gacacucaccaugcugaaatt

SEQ ID NO: 1171 - DNA, 21 bp, Artificial Sequence
  siRNA with 19 paired ribonucleotides and two unpaired nucleotides(“t”) on the 3′ end of each strand used in screens of DNA damaging agents camptothecin screen
aagggugacuuuguguccutt

SEQ ID NO: 1172 - DNA, 21 bp, Artificial Sequence
  siRNA with 19 paired ribonucleotides and two unpaired nucleotides(“t”) on the 3′ end of each strand used in screens of DNA damaging agents camptothecin screen
accaggaacaaaccuguugtt

SEQ ID NO: 1173 - DNA, 21 bp, Artificial Sequence
  siRNA with 19 paired ribonucleotides and two unpaired nucleotides(“t”) on the 3′ end of each strand used in screens of DNA damaging agents camptothecin screen
uuugaagguggcccuccuatt

SEQ ID NO: 1174 - DNA, 21 bp, Artificial Sequence
  siRNA with 19 paired ribonucleotides and two unpaired nucleotides(“t”) on the 3′ end of each strand used in screens of DNA damaging agents camptothecin screen
aaaucgagaaggaggcucatt

SEQ ID NO: 1175 - DNA, 21 bp, Artificial Sequence
  siRNA with 19 paired ribonucleotides and two unpaired nucleotides(“t”) on the 3′ end of each strand used in screens of DNA damaging agents camptothecin screen
auagugaccgucccuuugatt

SEQ ID NO: 1176 - DNA, 21 bp, Artificial Sequence
  siRNA with 19 paired ribonucleotides and two unpaired nucleotides(“t”) on the 3′ end of each strand used in screens of DNA damaging agents camptothecin screen
ccagguuccuccaaagaugtt

SEQ ID NO: 1177 - DNA, 21 bp, Artificial Sequence
  siRNA with 19 paired ribonucleotides and two unpaired nucleotides(“t”) on the 3′ end of each strand used in screens of DNA damaging agents camptothecin screen
aaggguucagcaucugacutt

SEQ ID NO: 1178 - DNA, 21 bp, Artificial Sequence
  siRNA with 19 paired ribonucleotides and two unpaired nucleotides(“t”) on the 3′ end of each strand used in screens of DNA damaging agents camptothecin screen
ccuggagacauugcaccagtt

SEQ ID NO: 1179 - DNA, 21 bp, Artificial Sequence
  siRNA with 19 paired ribonucleotides and two unpaired nucleotides(“t”) on the 3′ end of each strand used in screens of DNA damaging agents camptothecin screen
ggugcuaccuccuuuccagtt

SEQ ID NO: 1180 - DNA, 21 bp, Artificial Sequence
  siRNA with 19 paired ribonucleotides and two unpaired nucleotides(“t”) on the 3′ end of each strand used in screens of DNA damaging agents camptothecin screen
aguugcccacccuguuuuutt

SEQ ID NO: 1181 - DNA, 21 bp, Artificial Sequence
  siRNA with 19 paired ribonucleotides and two unpaired nucleotides(“t”) on the 3′ end of each strand used in screens of DNA damaging agents camptothecin screen
gaaagaauccguccgcaugtt

SEQ ID NO: 1182 - DNA, 21 bp, Artificial Sequence
  siRNA with 19 paired ribonucleotides and two unpaired nucleotides(“t”) on the 3′ end of each strand used in screens of DNA damaging agents camptothecin screen
gcagccagaacucucaaagtt

SEQ ID NO: 1183 - DNA, 21 bp, Artificial Sequence
  siRNA with 19 paired ribonucleotides and two unpaired nucleotides(“t”) on the 3′ end of each strand used in screens of DNA damaging agents camptothecin screen
guuguaggaggcagugaugtt

SEQ ID NO: 1184 - DNA, 21 bp, Artificial Sequence
  siRNA with 19 paired ribonucleotides and two unpaired nucleotides(“t”) on the 3′ end of each strand used in screens of DNA damaging agents camptothecin screen
ucucaauuuggaagucuugtt

SEQ ID NO: 1185 - DNA, 21 bp, Artificial Sequence
  siRNA with 19 paired ribonucleotides and two unpaired nucleotides(“t”) on the 3′ end of each strand used in screens of DNA damaging agents camptothecin screen
ugaucaaugaucggauuggtt

SEQ ID NO: 1186 - DNA, 21 bp, Artificial Sequence
  siRNA with 19 paired ribonucleotides and two unpaired nucleotides(“t”) on the 3′ end of each strand used in screens of DNA damaging agents camptothecin screen
accaggauuuggaguggautt

SEQ ID NO: 1187 - DNA, 21 bp, Artificial Sequence
  siRNA with 19 paired ribonucleotides and two unpaired nucleotides(“t”) on the 3′ end of each strand used in screens of DNA damaging agents camptothecin screen
caaggcauccguuauaucutt

SEQ ID NO: 1188 - DNA, 21 bp, Artificial Sequence
  siRNA with 19 paired ribonucleotides and two unpaired nucleotides(“t”) on the 3′ end of each strand used in screens of DNA damaging agents camptothecin screen
guggcuggauucauguucctt

SEQ ID NO: 1189 - DNA, 21 bp, Artificial Sequence
  siRNA with 19 paired ribonucleotides and two unpaired nucleotides(“t”) on the 3′ end of each strand used in screens of DNA damaging agents camptothecin screen
ccagauccuugucuggaagtt

SEQ ID NO: 1190 - DNA, 21 bp, Artificial Sequence
  siRNA with 19 paired ribonucleotides and two unpaired nucleotides(“t”) on the 3′ end of each strand used in screens of DNA damaging agents camptothecin screen
cgacaacaagcugcugguctt

SEQ ID NO: 1191 - DNA, 21 bp, Artificial Sequence
  siRNA with 19 paired ribonucleotides and two unpaired nucleotides(“t”) on the 3′ end of each strand used in screens of DNA damaging agents camptothecin screen
gaagcuguccauguuggagtt

SEQ ID NO: 1192 - DNA, 21 bp, Artificial Sequence
  siRNA with 19 paired ribonucleotides and two unpaired nucleotides(“t”) on the 3′ end of each strand used in screens of DNA damaging agents camptothecin screen
agccagcagauguaauuggtt

SEQ ID NO: 1193 - DNA, 21 bp, Artificial Sequence
  siRNA with 19 paired ribonucleotides and two unpaired nucleotides(“t”) on the 3′ end of each strand used in screens of DNA damaging agents camptothecin screen
cauuucaaugaggcuccagtt

SEQ ID NO: 1194 - DNA, 21 bp, Artificial Sequence
  siRNA with 19 paired ribonucleotides and two unpaired nucleotides(“t”) on the 3′ end of each strand used in screens of DNA damaging agents camptothecin screen
gucauuuacagaguggcuctt

SEQ ID NO: 1195 - DNA, 21 bp, Artificial Sequence
  siRNA with 19 paired ribonucleotides and two unpaired nucleotides(“t”) on the 3′ end of each strand used in screens of DNA damaging agents camptothecin screen
aggacacuuuggguaccagtt

SEQ ID NO: 1196 - DNA, 21 bp, Artificial Sequence
  siRNA with 19 paired ribonucleotides and two unpaired nucleotides(“t”) on the 3′ end of each strand used in screens of DNA damaging agents camptothecin screen
gacccuaaagaucguccuutt

SEQ ID NO: 1197 - DNA, 21 bp, Artificial Sequence
  siRNA with 19 paired ribonucleotides and two unpaired nucleotides(“t”) on the 3′ end of each strand used in screens of DNA damaging agents camptothecin screen
gcugaggagaauaugccuctt

SEQ ID NO: 1198 - DNA, 21 bp, Artificial Sequence
  siRNA with 19 paired ribonucleotides and two unpaired nucleotides(“t”) on the 3′ end of each strand used in screens of DNA damaging agents camptothecin screen
caaaguuauuagccccaagtt

SEQ ID NO: 1199 - DNA, 21 bp, Artificial Sequence
  siRNA with 19 paired ribonucleotides and two unpaired nucleotides(“t”) on the 3′ end of each strand used in screens of DNA damaging agents camptothecin screen
cagaggccaaguauaucaatt

SEQ ID NO: 1200 - DNA, 21 bp, Artificial Sequence
  siRNA with 19 paired ribonucleotides and two unpaired nucleotides(“t”) on the 3′ end of each strand used in screens of DNA damaging agents camptothecin screen
ccugcagauuugcacagcgtt

SEQ ID NO: 1201 - DNA, 21 bp, Artificial Sequence
  siRNA with 19 paired ribonucleotides and two unpaired nucleotides(“t”) on the 3′ end of each strand used in screens of DNA damaging agents camptothecin screen
auccuggagaugaccgugatt

SEQ ID NO: 1202 - DNA, 21 bp, Artificial Sequence
  siRNA with 19 paired ribonucleotides and two unpaired nucleotides(“t”) on the 3′ end of each strand used in screens of DNA damaging agents camptothecin screen
gccggucauggagaagcggtt

SEQ ID NO: 1203 - DNA, 21 bp, Artificial Sequence
  siRNA with 19 paired ribonucleotides and two unpaired nucleotides(“t”) on the 3′ end of each strand used in screens of DNA damaging agents camptothecin screen
uggcccugagacugcaucgtt

SEQ ID NO: 1204 - DNA, 21 bp, Artificial Sequence
  siRNA with 19 paired ribonucleotides and two unpaired nucleotides(“t”) on the 3′ end of each strand used in screens of DNA damaging agents camptothecin screen
ccccuccaugcucagaacutt

SEQ ID NO: 1205 - DNA, 21 bp, Artificial Sequence
  siRNA with 19 paired ribonucleotides and two unpaired nucleotides(“t”) on the 3′ end of each strand used in screens of DNA damaging agents camptothecin screen
ccuaucugggaagccugugtt

SEQ ID NO: 1206 - DNA, 21 bp, Artificial Sequence
  siRNA with 19 paired ribonucleotides and two unpaired nucleotides(“t”) on the 3′ end of each strand used in screens of DNA damaging agents camptothecin screen
ugccccagugacaauaacatt

SEQ ID NO: 1207 - DNA, 21 bp, Artificial Sequence
  siRNA with 19 paired ribonucleotides and two unpaired nucleotides(“t”) on the 3′ end of each strand used in screens of DNA damaging agents camptothecin screen
agagagcuggaccauucautt

SEQ ID NO: 1208 - DNA, 21 bp, Artificial Sequence
  siRNA with 19 paired ribonucleotides and two unpaired nucleotides(“t”) on the 3′ end of each strand used in screens of DNA damaging agents camptothecin screen
augagcaaugcggauagcutt

SEQ ID NO: 1209 - DNA, 21 bp, Artificial Sequence
  siRNA with 19 paired ribonucleotides and two unpaired nucleotides(“t”) on the 3′ end of each strand used in screens of DNA damaging agents camptothecin screen
gccaugugucugaugacautt

SEQ ID NO: 1210 - DNA, 21 bp, Artificial Sequence
  siRNA with 19 paired ribonucleotides and two unpaired nucleotides(“t”) on the 3′ end of each strand used in screens of DNA damaging agents camptothecin screen
agacaaagaggggaccuuctt

SEQ ID NO: 1211 - DNA, 21 bp, Artificial Sequence
  siRNA with 19 paired ribonucleotides and two unpaired nucleotides(“t”) on the 3′ end of each strand used in screens of DNA damaging agents camptothecin screen
gaaagucuauccgaaggcutt

SEQ ID NO: 1212 - DNA, 21 bp, Artificial Sequence
  siRNA with 19 paired ribonucleotides and two unpaired nucleotides(“t”) on the 3′ end of each strand used in screens of DNA damaging agents camptothecin screen
ugccucccugaaacuucgatt

SEQ ID NO: 1213 - DNA, 21 bp, Artificial Sequence
  siRNA with 19 paired ribonucleotides and two unpaired nucleotides(“t”) on the 3′ end of each strand used in screens of DNA damaging agents camptothecin screen
agguaugcaucgugcagaatt

SEQ ID NO: 1214 - DNA, 21 bp, Artificial Sequence
  siRNA with 19 paired ribonucleotides and two unpaired nucleotides(“t”) on the 3′ end of each strand used in screens of DNA damaging agents camptothecin screen
gcaaucaaaccgucaugactt

SEQ ID NO: 1215 - DNA, 21 bp, Artificial Sequence
  siRNA with 19 paired ribonucleotides and two unpaired nucleotides(“t”) on the 3′ end of each strand used in screens of DNA damaging agents camptothecin screen
uauccugcuggaugaacactt

SEQ ID NO: 1216 - DNA, 21 bp, Artificial Sequence
  siRNA with 19 paired ribonucleotides and two unpaired nucleotides(“t”) on the 3′ end of each strand used in screens of DNA damaging agents camptothecin screen
cauuguccacugugacuugtt

SEQ ID NO: 1217 - DNA, 21 bp, Artificial Sequence
  siRNA with 19 paired ribonucleotides and two unpaired nucleotides(“t”) on the 3′ end of each strand used in screens of DNA damaging agents camptothecin screen
ugaaguggccauucugcagtt

SEQ ID NO: 1218 - DNA, 21 bp, Artificial Sequence
  siRNA with 19 paired ribonucleotides and two unpaired nucleotides(“t”) on the 3′ end of each strand used in screens of DNA damaging agents camptothecin screen
uguggacauugccacuguctt

SEQ ID NO: 1219 - DNA, 21 bp, Artificial Sequence
  siRNA with 19 paired ribonucleotides and two unpaired nucleotides(“t”) on the 3′ end of each strand used in screens of DNA damaging agents camptothecin screen
augaucgcaccgcagaggutt

SEQ ID NO: 1220 - DNA, 21 bp, Artificial Sequence
  siRNA with 19 paired ribonucleotides and two unpaired nucleotides(“t”) on the 3′ end of each strand used in screens of DNA damaging agents camptothecin screen
uacaugacguacuugagugtt

SEQ ID NO: 1221 - DNA, 21 bp, Artificial Sequence
  siRNA with 19 paired ribonucleotides and two unpaired nucleotides(“t”) on the 3′ end of each strand used in screens of DNA damaging agents camptothecin screen
ugcuaaggggaucggacautt

SEQ ID NO: 1222 - DNA, 21 bp, Artificial Sequence
  siRNA with 19 paired ribonucleotides and two unpaired nucleotides(“t”) on the 3′ end of each strand used in screens of DNA damaging agents camptothecin screen
accacuccggauacaucactt

SEQ ID NO: 1223 - DNA, 21 bp, Artificial Sequence
  siRNA with 19 paired ribonucleotides and two unpaired nucleotides(“t”) on the 3′ end of each strand used in screens of DNA damaging agents camptothecin screen
acuaaggcgucugcgagautt

SEQ ID NO: 1224 - DNA, 21 bp, Artificial Sequence
  siRNA with 19 paired ribonucleotides and two unpaired nucleotides(“t”) on the 3′ end of each strand used in screens of DNA damaging agents camptothecin screen
ggaccucacagcaacucuutt

SEQ ID NO: 1225 - DNA, 21 bp, Artificial Sequence
  siRNA with 19 paired ribonucleotides and two unpaired nucleotides(“t”) on the 3′ end of each strand used in screens of DNA damaging agents camptothecin screen
cugguugcaguuccauucctt

SEQ ID NO: 1226 - DNA, 21 bp, Artificial Sequence
  siRNA with 19 paired ribonucleotides and two unpaired nucleotides(“t”) on the 3′ end of each strand used in screens of DNA damaging agents camptothecin screen
gugagcauccuggaucaaatt

SEQ ID NO: 1227 - DNA, 21 bp, Artificial Sequence
  siRNA with 19 paired ribonucleotides and two unpaired nucleotides(“t”) on the 3′ end of each strand used in screens of DNA damaging agents camptothecin screen
uucagagaguccacacacctt

SEQ ID NO: 1228 - DNA, 21 bp, Artificial Sequence
  siRNA with 19 paired ribonucleotides and two unpaired nucleotides(“t”) on the 3′ end of each strand used in screens of DNA damaging agents camptothecin screen
aaaacccccgggagucguctt

SEQ ID NO: 1229 - DNA, 21 bp, Artificial Sequence
  siRNA with 19 paired ribonucleotides and two unpaired nucleotides(“t”) on the 3′ end of each strand used in screens of DNA damaging agents camptothecin screen
aguggcaccaaguggcuggtt

SEQ ID NO: 1230 - DNA, 21 bp, Artificial Sequence
  siRNA with 19 paired ribonucleotides and two unpaired nucleotides(“t”) on the 3′ end of each strand used in screens of DNA damaging agents camptothecin screen
gaaaccugcuuugucauuutt

SEQ ID NO: 1231 - DNA, 21 bp, Artificial Sequence
  siRNA with 19 paired ribonucleotides and two unpaired nucleotides(“t”) on the 3′ end of each strand used in screens of DNA damaging agents camptothecin screen
cugaugcacuuugcugcagtt

SEQ ID NO: 1232 - DNA, 21 bp, Artificial Sequence
  siRNA with 19 paired ribonucleotides and two unpaired nucleotides(“t”) on the 3′ end of each strand used in screens of DNA damaging agents camptothecin screen
cugcagguucaaaucccagtt

SEQ ID NO: 1233 - DNA, 21 bp, Artificial Sequence
  siRNA with 19 paired ribonucleotides and two unpaired nucleotides(“t”) on the 3′ end of each strand used in screens of DNA damaging agents camptothecin screen
ggggaaaaagcuuugcguutt

SEQ ID NO: 1234 - DNA, 21 bp, Artificial Sequence
  siRNA with 19 paired ribonucleotides and two unpaired nucleotides(“t”) on the 3′ end of each strand used in screens of DNA damaging agents camptothecin screen
uaucgagccaccauuugugtt

SEQ ID NO: 1235 - DNA, 21 bp, Artificial Sequence
  siRNA with 19 paired ribonucleotides and two unpaired nucleotides(“t”) on the 3′ end of each strand used in screens of DNA damaging agents camptothecin screen
ugaugcauauagccgcaactt

SEQ ID NO: 1236 - DNA, 21 bp, Artificial Sequence
  siRNA with 19 paired ribonucleotides and two unpaired nucleotides(“t”) on the 3′ end of each strand used in screens of DNA damaging agents camptothecin screen
ugcacagggccaaaguugatt

SEQ ID NO: 1237 - DNA, 21 bp, Artificial Sequence
  siRNA with 19 paired ribonucleotides and two unpaired nucleotides(“t”) on the 3′ end of each strand used in screens of DNA damaging agents camptothecin screen
uacuugaaggugggcccautt

SEQ ID NO: 1238 - DNA, 21 bp, Artificial Sequence
  siRNA with 19 paired ribonucleotides and two unpaired nucleotides(“t”) on the 3′ end of each strand used in screens of DNA damaging agents camptothecin screen
gaaaucaguugcugagggctt

SEQ ID NO: 1239 - DNA, 21 bp, Artificial Sequence
  siRNA with 19 paired ribonucleotides and two unpaired nucleotides(“t”) on the 3′ end of each strand used in screens of DNA damaging agents camptothecin screen
accuaggaccgucuugcuutt